SEQUENCE LISTING (1012 sequences)

SEQ ID NO: 1 - RNA, 80 nt, synthetic construct
  misc_feature 1..80: note = Synthetic
  variation 1..80: note = internal linkers L1 and L2 are present or absent
  source 1..80: mol_type = other RNA
acgcaaatatcagtccagcggttttagagctatagcaagttaaaataaggcgtccgttatcacgggcaccgagtcggtgc

SEQ ID NO: 2 - RNA, 78 nt, synthetic construct
  misc_feature 1..78: note = Synthetic
  variation 1..78: note = internal linkers L1 and L2 are present or absent
  source 1..78: mol_type = other RNA
acgcaaatatcagtccagcggttttagagctatagcaagttaaaataaggcgtccgttatcaggcaccgagtcggtgc

SEQ ID NO: 3 - RNA, 78 nt, synthetic construct
  misc_feature 1..78: note = Synthetic
  variation 1..78: note = internal linkers L1 and L2 are present or absent
  source 1..78: mol_type = other RNA
acgcaaatatcagtccagcggttttagagctagcaagttaaaataaggcgtccgttatcacgggcaccgagtcggtgc

SEQ ID NO: 4 - RNA, 76 nt, synthetic construct
  misc_feature 1..76: note = Synthetic
  variation 1..76: note = internal linkers L1 and L2 are present or absent
  source 1..76: mol_type = other RNA
acgcaaatatcagtccagcggttttagagctagcaagttaaaataaggcgtccgttatcaggcaccgagtcggtgc

SEQ ID NO: 5 - RNA, 80 nt, synthetic construct
  misc_feature 1..80: note = Synthetic
  variation 1..80: note = internal linkers L1 and L2 are present or absent
  source 1..80: mol_type = other RNA
acacaaataccagtccagcggttttagagctatagcaagttaaaataaggcgtccgttatcacgggcaccgagtcggtgc

SEQ ID NO: 6 - RNA, 78 nt, synthetic construct
  misc_feature 1..78: note = Synthetic
  variation 1..78: note = internal linkers L1 and L2 are present or absent
  source 1..78: mol_type = other RNA
acacaaataccagtccagcggttttagagctatagcaagttaaaataaggcgtccgttatcaggcaccgagtcggtgc

SEQ ID NO: 7 - RNA, 78 nt, synthetic construct
  misc_feature 1..78: note = Synthetic
  variation 1..78: note = internal linkers L1 and L2 are present or absent
  source 1..78: mol_type = other RNA
acacaaataccagtccagcggttttagagctagcaagttaaaataaggcgtccgttatcacgggcaccgagtcggtgc

SEQ ID NO: 8 - RNA, 76 nt, synthetic construct
  misc_feature 1..76: note = Synthetic
  variation 1..76: note = internal linkers L1 and L2 are present or absent
  source 1..76: mol_type = other RNA
acacaaataccagtccagcggttttagagctagcaagttaaaataaggcgtccgttatcaggcaccgagtcggtgc

SEQ ID NO: 9 - RNA, 90 nt, synthetic construct
  misc_feature 1..90: note = Synthetic
  source 1..90: mol_type = other RNA
acgcaaatatcagtccagcggttttagagctagaaatagcaagttaaaataaggctagtccgttatcacgaaagggcaccgagtcggtgc

SEQ ID NO: 10 - RNA, 90 nt, synthetic construct
  misc_feature 1..90: note = Synthetic
  source 1..90: mol_type = other RNA
acacaaataccagtccagcggttttagagctagaaatagcaagttaaaataaggctagtccgttatcacgaaagggcaccgagtcggtgc

SEQ ID NO: 11 - RNA, 100 nt, synthetic construct
  misc_feature 1..100: note = Synthetic
  source 1..100: mol_type = other RNA
acacaaataccagtccagcggttttagagctagaaatagcaagttaaaataaggctagtccgttatcaacttgaaaaagtggcaccgagtcggtgctttt

SEQ ID NO: 12 - RNA, 100 nt, synthetic construct
  misc_feature 1..100: note = Synthetic
  source 1..100: mol_type = other RNA
acgcaaatatcagtccagcggttttagagctagaaatagcaagttaaaataaggctagtccgttatcaacttgaaaaagtggcaccgagtcggtgctttt

SEQ ID NO: 13 - RNA, 88 nt, synthetic construct
  misc_feature 1..88: note = Synthetic
  source 1..88: mol_type = other RNA
acacaaataccagtccagcggttttagagctagaaatagcaagttaaaataaggctagtccgttatcaacttggcaccgagtcggtgc

SEQ ID NO: 14 - RNA, 88 nt, synthetic construct
  misc_feature 1..88: note = Synthetic
  source 1..88: mol_type = other RNA
acacaaataccagtccagcggttttagagctagaaatagcaagttaaaataaggctagtccgttatcacaagggcaccgagtcggtgc

SEQ ID NO: 15 - skipped

SEQ ID NO: 16 - protein, 7 aa, synthetic construct
  REGION 1..7: note = Synthetic
PKKKRKV

SEQ ID NO: 17 - protein, 7 aa, synthetic construct
  REGION 1..7: note = Synthetic
PKKKRRV

SEQ ID NO: 18 - protein, 16 aa, synthetic construct
  REGION 1..16: note = Synthetic
KRPAATKKAGQAKKKK

SEQ ID NO: 19 - protein, 7 aa, synthetic construct
  REGION 1..7: note = Synthetic
PKKKRKV

SEQ ID NO: 20 - RNA, 86 nt, synthetic construct
  misc_feature 1..86: note = Synthetic,
  variation 1..86: note = internal linker L2 is present or absent
  source 1..86: mol_type = other RNA
acacaaataccagtccagcggttttagagctagtagcaagttaaaataaggctagtccgttatcaacttggcaccgagtcggtgct

SEQ ID NO: 21 - RNA, 85 nt, synthetic construct
  misc_feature 1..85: note = Synthetic
  variation 1..85: note = internal linker L2 is present or absent
  source 1..85: mol_type = other RNA
acacaaataccagtccagcggttttagagctatagcaagttaaaataaggctagtccgttatcaacttggcaccgagtcggtgct

SEQ ID NO: 22 - RNA, 84 nt, synthetic construct
  misc_feature 1..84: note = Synthetic
  variation 1..84: note = internal linker L2 is present or absent
  source 1..84: mol_type = other RNA
acacaaataccagtccagcggttttagagctgagcaagttaaaataaggctagtccgttatcaacttggcaccgagtcggtgct

SEQ ID NO: 23 - RNA, 83 nt, synthetic construct
  misc_feature 1..83: note = Synthetic
  variation 1..83: note = internal linker L2 is present or absent
  source 1..83: mol_type = other RNA
acacaaataccagtccagcggttttagagctagcaagttaaaataaggctagtccgttatcaacttggcaccgagtcggtgct

SEQ ID NO: 24 - RNA, 81 nt, synthetic construct
  misc_feature 1..81: note = Synthetic
  variation 1..81: note = internal linker L2 is present or absent
  source 1..81: mol_type = other RNA
acacaaataccagtccagcggttttagagcgcaagttaaaataaggctagtccgttatcaacttggcaccgagtcggtgct

SEQ ID NO: 25 - RNA, 82 nt, synthetic construct
  misc_feature 1..82: note = Synthetic
  variation 1..82: note = internal linker L2 is present or absent
  source 1..82: mol_type = other RNA
acacaaataccagtccagcggttttagagcggcaagttaaaataaggctagtccgttatcaacttggcaccgagtcggtgct

SEQ ID NO: 26 - RNA, 79 nt, synthetic construct
  misc_feature 1..79: note = Synthetic
  variation 1..79: note = internal linker L2 is present or absent
  source 1..79: mol_type = other RNA
acacaaataccagtccagcggttttagagcaagttaaaataaggctagtccgttatcaacttggcaccgagtcggtgct

SEQ ID NO: 27 - RNA, 86 nt, synthetic construct; identical to SEQ ID NO: 20
  misc_feature 1..86: note = Synthetic
  variation 1..86: note = internal linker L2 is present or absent
  source 1..86: mol_type = other RNA

SEQ ID NO: 28 - RNA, 85 nt, synthetic construct; identical to SEQ ID NO: 21
  misc_feature 1..85: note = Synthetic
  variation 1..85: note = internal linker L2 is present or absent
  source 1..85: mol_type = other RNA

SEQ ID NO: 29 - RNA, 84 nt, synthetic construct; identical to SEQ ID NO: 22
  misc_feature 1..84: note = Synthetic
  variation 1..84: note = internal linker L2 is present or absent
  source 1..84: mol_type = other RNA

SEQ ID NO: 30 - RNA, 83 nt, synthetic construct; identical to SEQ ID NO: 23
  misc_feature 1..83: note = Synthetic
  variation 1..83: note = internal linker L2 is present or absent
  source 1..83: mol_type = other RNA

SEQ ID NO: 31 - RNA, 82 nt, synthetic construct; identical to SEQ ID NO: 25
  misc_feature 1..82: note = Synthetic
  variation 1..82: note = internal linker L2 is present or absent
  source 1..82: mol_type = other RNA

SEQ ID NO: 32 - RNA, 81 nt, synthetic construct; identical to SEQ ID NO: 24
  misc_feature 1..81: note = Synthetic
  variation 1..81: note = internal linker L2 is present or absent
  source 1..81: mol_type = other RNA

SEQ ID NO: 33 - RNA, 79 nt, synthetic construct; identical to SEQ ID NO: 26
  misc_feature 1..79: note = Synthetic
  variation 1..79: note = internal linker L2 is present or absent
  source 1..79: mol_type = other RNA

SEQ ID NO: 34 - RNA, 87 nt, synthetic construct
  misc_feature 1..87: note = Synthetic
  variation 1..87: note = internal linker L4
  source 1..87: mol_type = other RNA
acacaaataccagtccagcggttttagagctagaaatagcaagttaaaataaggcgtccgttatcaacttggcaccgagtcggtgct

SEQ ID NO: 35 - RNA, 87 nt, synthetic construct; identical to SEQ ID NO: 34
  misc_feature 1..87: note = Synthetic
  variation 1..87: note = internal linker L2 is present or absent
  source 1..87: mol_type = other RNA

SEQ ID NO: 36 - RNA, 87 nt, synthetic construct; identical to SEQ ID NO: 34
  misc_feature 1..87: note = Synthetic
  variation 1..87: note = internal linker L1 is present or absent
  source 1..87: mol_type = other RNA

SEQ ID NO: 37 - RNA, 88 nt, synthetic construct
  misc_feature 1..88: note = Synthetic
  variation 1..88: note = internal linker L2 is present or absent
  source 1..88: mol_type = other RNA
acacaaataccagtccagcggttttagagctagaaatagcaagttaaaataaggctagtccgttatcaactggcaccgagtcggtgct

SEQ ID NO: 38 - RNA, 87 nt, synthetic construct
  misc_feature 1..87: note = Synthetic
  variation 1..87: note = internal linker L2 is present or absent
  source 1..87: mol_type = other RNA
acacaaataccagtccagcggttttagagctagaaatagcaagttaaaataaggctagtccgttatcaacggcaccgagtcggtgct

SEQ ID NO: 39 - RNA, 87 nt, synthetic construct
  misc_feature 1..87: note = Synthetic
  variation 1..87: note = internal linker L2 is present or absent
  source 1..87: mol_type = other RNA
acacaaataccagtccagcggttttagagctagaaatagcaagttaaaataaggctagtccgttatcaatggcaccgagtcggtgct

SEQ ID NO: 40 - RNA, 86 nt, synthetic construct
  misc_feature 1..86: note = Synthetic
  variation 1..86: note = internal linker L2 is present or absent
  source 1..86: mol_type = other RNA
acacaaataccagtccagcggttttagagctagaaatagcaagttaaaataaggctagtccgttatcaaggcaccgagtcggtgct

SEQ ID NO: 41 - RNA, 88 nt, synthetic construct; identical to SEQ ID NO: 37
  misc_feature 1..88: note = Synthetic
  variation 1..88: note = internal linker L1 is present or absent
  source 1..88: mol_type = other RNA

SEQ ID NO: 42 - RNA, 87 nt, synthetic construct; identical to SEQ ID NO: 38
  misc_feature 1..87: note = Synthetic
  variation 1..87: note = internal linker L1 is present or absent
  source 1..87: mol_type = other RNA

SEQ ID NO: 43 - RNA, 87 nt, synthetic construct; identical to SEQ ID NO: 39
  misc_feature 1..87: note = Synthetic
  variation 1..87: note = internal linker L1 is present or absent
  source 1..87: mol_type = other RNA

SEQ ID NO: 44 - RNA, 86 nt, synthetic construct; identical to SEQ ID NO: 40
  misc_feature 1..86: note = Synthetic
  variation 1..86: note = internal linker L1 is present or absent
  source 1..86: mol_type = other RNA

SEQ ID NO: 45 - RNA, 83 nt, synthetic construct
  misc_feature 1..83: note = Synthetic
  variation 1..83: note = internal linkers L1 and L2 are present or absent
  source 1..83: mol_type = other RNA
acacaaataccagtccagcggttttagagctatagcaagttaaaataaggcgtccgttatcaacttggcaccgagtcggtgct

SEQ ID NO: 46 - RNA, 83 nt, synthetic construct; identical to SEQ ID NO: 45
  misc_feature 1..83: note = Synthetic
  variation 1..83: note = internal linker L1 is present or absent
  source 1..83: mol_type = other RNA

SEQ ID NO: 47 - RNA, 83 nt, synthetic construct
  misc_feature 1..83: note = Synthetic
  variation 1..83: note = internal linkers L1 and L2 are present or absent
  source 1..83: mol_type = other RNA
acacaaataccagtccagcggttttagagctatagcaagttaaaataaggctagtccgttatcaatggcaccgagtcggtgct

SEQ ID NO: 48 - RNA, 83 nt, synthetic construct; identical to SEQ ID NO: 47
  misc_feature 1..83: note = Synthetic
  variation 1..83: note = internal linker L1 is present or absent
  source 1..83: mol_type = other RNA

SEQ ID NO: 49 - RNA, 81 nt, synthetic construct
  misc_feature 1..81: note = Synthetic
  variation 1..81: note = internal linkers L1 and L2 are present or absent
  source 1..81: mol_type = other RNA
acacaaataccagtccagcggttttagagctatagcaagttaaaataaggcgtccgttatcaatggcaccgagtcggtgct

SEQ ID NO: 50 - RNA, 81 nt, synthetic construct; identical to SEQ ID NO: 49
  misc_feature 1..81: note = Synthetic
  variation 1..81: note = internal linker L1 is present or absent
  source 1..81: mol_type = other RNA

SEQ ID NO: 51 - RNA, 82 nt, synthetic construct
  misc_feature 1..82: note = Synthetic
  variation 1..82: note = internal linkers L1 and L2 are present or absent
  source 1..82: mol_type = other RNA
acacaaataccagtccagcggttttagagctagtagcaagttaaaataaggcgtccgttatcaatggcaccgagtcggtgct

SEQ ID NO: 52 - RNA, 82 nt, synthetic construct; identical to SEQ ID NO: 51
  misc_feature 1..82: note = Synthetic
  variation 1..82: note = internal linker L1 is present or absent
  source 1..82: mol_type = other RNA

SEQ ID NO: 53 - RNA, 79 nt, synthetic construct
  misc_feature 1..79: note = Synthetic
  variation 1..79: note = internal linkers L1 and L2 are present or absent
  source 1..79: mol_type = other RNA
acacaaataccagtccagcggttttagagctagcaagttaaaataaggcgtccgttatcaatggcaccgagtcggtgct

SEQ ID NO: 54 - RNA, 79 nt, synthetic construct; identical to SEQ ID NO: 53
  misc_feature 1..79: note = Synthetic
  variation 1..79: note = internal linker L1 is present or absent
  source 1..79: mol_type = other RNA

SEQ ID NO: 55 - RNA, 80 nt, synthetic construct
  misc_feature 1..80: note = Synthetic
  variation 1..80: note = internal linkers L1 and L2 are present or absent
  source 1..80: mol_type = other RNA
acacaaataccagtccagcggttttagagctgagcaagttaaaataaggcgtccgttatcaatggcaccgagtcggtgct

SEQ ID NO: 56 - RNA, 80 nt, synthetic construct; identical to SEQ ID NO: 55
  misc_feature 1..80: note = Synthetic
  variation 1..80: note = internal linker L1 is present or absent
  source 1..80: mol_type = other RNA

SEQ ID NO: 57 - RNA, 77 nt, synthetic construct
  misc_feature 1..77: note = Synthetic
  variation 1..77: note = internal linkers L1 and L2 are present or absent
  source 1..77: mol_type = other RNA
acacaaataccagtccagcggttttagagcgcaagttaaaataaggcgtccgttatcaatggcaccgagtcggtgct

SEQ ID NO: 58 - RNA, 77 nt, synthetic construct; identical to SEQ ID NO: 57
  misc_feature 1..77: note = Synthetic
  variation 1..77: note = internal linker L1 is present or absent
  source 1..77: mol_type = other RNA

SEQ ID NO: 59 - RNA, 87 nt, synthetic construct
  misc_feature 1..87: note = Synthetic
  variation 1..87: note = internal linker L2 is present or absent
  source 1..87: mol_type = other RNA
acacaaataccagtccagcggttttagagctagaaatagcaagttaaaataaggctagtccgttatcacgggcaccgagtcggtgct

SEQ ID NO: 60 - RNA, 87 nt, synthetic construct; identical to SEQ ID NO: 59
  misc_feature 1..87: note = Synthetic
  variation 1..87: note = internal linker L1 is present or absent
  source 1..87: mol_type = other RNA

SEQ ID NO: 61 - RNA, 85 nt, synthetic construct
  misc_feature 1..85: note = Synthetic
  variation 1..85: note = internal linker L1 is present or absent
  source 1..85: mol_type = other RNA
acacaaataccagtccagcggttttagagctagaaatagcaagttaaaataaggctagtccgttatcaggcaccgagtcggtgct

SEQ ID NO: 62 - RNA, 85 nt, synthetic construct; identical to SEQ ID NO: 61
  misc_feature 1..85: note = Synthetic
  variation 1..85: note = internal linker L2 is present or absent
  source 1..85: mol_type = other RNA

SEQ ID NO: 63 - RNA, 90 nt, synthetic construct; identical to SEQ ID NO: 10
  misc_feature 1..90: note = Synthetic
  source 1..90: mol_type = other RNA

SEQ ID NO: 64 - RNA, 90 nt, synthetic construct; identical to SEQ ID NO: 10
  misc_feature 1..90: note = Synthetic
  source 1..90: mol_type = other RNA

SEQ ID NO: 65 - RNA, 86 nt, synthetic construct
  misc_feature 1..86: note = Synthetic
  variation 1..86: note = internal linker L1 is present or absent
  source 1..86: mol_type = other RNA
acacaaataccagtccagcggttttagagctatagcaagttaaaataaggctagtccgttatcacgaaagggcaccgagtcggtgc

SEQ ID NO: 66 - RNA, 86 nt, synthetic construct
  misc_feature 1..86: note = Synthetic
  variation 1..86: note = internal linker L1 is present or absent
  source 1..86: mol_type = other RNA
acacaaataccagtccagcggttttagagctagaaatagcaagttaaaataaggctagtccgttatcacgggcaccgagtcggtgc

SEQ ID NO: 67 - RNA, 80 nt, synthetic construct; identical to SEQ ID NO: 5
  misc_feature 1..80: note = Synthetic
  variation 1..80: note = internal linker L1 is present or absent
  source 1..80: mol_type = other RNA

SEQ ID NO: 68 - RNA, 82 nt, synthetic construct
  misc_feature 1..82: note = Synthetic
  variation 1..82: note = internal linker L1 is present or absent
  source 1..82: mol_type = other RNA
acacaaataccagtccagcggttttagagctatagcaagttaaaataaggctagtccgttatcacgggcaccgagtcggtgc

SEQ ID NO: 69 - RNA, 86 nt, synthetic construct; identical to SEQ ID NO: 65
  misc_feature 1..86: note = Synthetic
  variation 1..86: note = internal linker L2 is present or absent
  source 1..86: mol_type = other RNA

SEQ ID NO: 70 - RNA, 88 nt, synthetic construct
  misc_feature 1..88: note = Synthetic
  source 1..88: mol_type = other RNA
acacaaataccagtccagcggttttagagctagaaatagcaagttaaaataaggctagtccgttatcaaaatggcaccgagtcggtgc

SEQ ID NO: 71 - RNA, 88 nt, synthetic construct; identical to SEQ ID NO: 70
  misc_feature 1..88: note = Synthetic
  source 1..88: mol_type = other RNA

SEQ ID NO: 72 - RNA, 84 nt, synthetic construct
  misc_feature 1..84: note = Synthetic
  variation 1..84: note = internal linker L1 is present or absent
  source 1..84: mol_type = other RNA
acacaaataccagtccagcggttttagagctatagcaagttaaaataaggctagtccgttatcaaaatggcaccgagtcggtgc

SEQ ID NO: 73 - RNA, 86 nt, synthetic construct
  misc_feature 1..86: note = Synthetic
  variation 1..86: note = internal linker L1 is present or absent
  source 1..86: mol_type = other RNA
acacaaataccagtccagcggttttagagctagaaatagcaagttaaaataaggctagtccgttatcaatggcaccgagtcggtgc

SEQ ID NO: 74 - RNA, 82 nt, synthetic construct
  misc_feature 1..82: note = Synthetic
  variation 1..82: note = internal linker L1 is present or absent
  source 1..82: mol_type = other RNA
acacaaataccagtccagcggttttagagctatagcaagttaaaataaggctagtccgttatcaatggcaccgagtcggtgc

SEQ ID NO: 75 - RNA, 76 nt, synthetic construct
  misc_feature 1..76: note = Synthetic
  variation 1..76: note = internal linker L1 is present or absent
  source 1..76: mol_type = other RNA
acacaaataccagtccagcggttttagagcgcaagttaaaataaggcgtccgttatcaatggcaccgagtcggtgc

SEQ ID NO: 76 - skipped

SEQ ID NO: 77 - RNA, 75 nt, synthetic construct
  misc_feature 1..75: note = Synthetic
  variation 1..75: note = internal linker L1, dS site is present or absent
  source 1..75: mol_type = other RNA
ggcccagactgagcacgtgagttttagaaagttaaaataaggctagtccgttatcacgggcaccgagtcggtgct

SEQ ID NO: 78 - RNA, 75 nt, synthetic construct; identical to SEQ ID NO: 77
  misc_feature 1..75: note = Synthetic
  variation 1..75: note = internal linkers L4 and L1
  source 1..75: mol_type = other RNA

SEQ ID NO: 79 - RNA, 75 nt, synthetic construct; identical to SEQ ID NO: 77
  misc_feature 1..75: note = Synthetic
  variation 1..75: note = internal linkers L3 and L1 are present or absent
  source 1..75: mol_type = other RNA

SEQ ID NO: 80 - RNA, 75 nt, synthetic construct; identical to SEQ ID NO: 77
  misc_feature 1..75: note = Synthetic
  variation 1..75: note = internal linkers L2 and L1 are present or absent
  source 1..75: mol_type = other RNA

SEQ ID NO: 81 - RNA, 75 nt, synthetic construct; identical to SEQ ID NO: 77
  misc_feature 1..75: note = Synthetic
  variation 1..75: note = internal linker L1 is present or absent
  source 1..75: mol_type = other RNA

SEQ ID NO: 82 - RNA, 79 nt, synthetic construct
  misc_feature 1..79: note = Synthetic
  variation 1..79: note = internal linker L1 is present or absent
  source 1..79: mol_type = other RNA
ggcccagactgagcacgtgagttttagagcgcaagttaaaataaggctagtccgttatcacgggcaccgagtcggtgct

SEQ ID NO: 83 - RNA, 83 nt, synthetic construct
  misc_feature 1..83: note = Synthetic
  variation 1..83: note = internal linker L1 is present or absent
  source 1..83: mol_type = other RNA
ggcccagactgagcacgtgagttttagagctatagcaagttaaaataaggctagtccgttatcacgggcaccgagtcggtgct

SEQ ID NO: 84 - RNA, 83 nt, synthetic construct; identical to SEQ ID NO: 83
  misc_feature 1..83: note = Synthetic
  variation 1..83: note = internal linker L1 is present or absent
  source 1..83: mol_type = other RNA

SEQ ID NO: 85-100 - skipped (16 entries)

SEQ ID NO: 101 - RNA, 80 nt, synthetic construct; identical to SEQ ID NO: 1
  misc_feature 1..80: note = Synthetic
  variation 1..80: note = internal linkers L1 and L2, 2'-O-Me and PS linkage modifications are present or absent
  source 1..80: mol_type = other RNA

SEQ ID NO: 102 - RNA, 78 nt, synthetic construct; identical to SEQ ID NO: 2
  misc_feature 1..78: note = Synthetic
  variation 1..78: note = internal linkers L1 and L2, 2'-O-Me and PS linkage modifications are present or absent
  source 1..78: mol_type = other RNA

SEQ ID NO: 103 - RNA, 78 nt, synthetic construct; identical to SEQ ID NO: 3
  misc_feature 1..78: note = Synthetic
  variation 1..78: note = internal linkers L1 and L2, 2'-O-Me and PS linkage modifications are present or absent
  source 1..78: mol_type = other RNA

SEQ ID NO: 104 - RNA, 76 nt, synthetic construct; identical to SEQ ID NO: 4
  misc_feature 1..76: note = Synthetic
  variation 1..76: note = internal linkers L1 and L2, 2'-O-Me and PS linkage modifications are present or absent
  source 1..76: mol_type = other RNA

SEQ ID NO: 105 - RNA, 80 nt, synthetic construct; identical to SEQ ID NO: 5
  misc_feature 1..80: note = Synthetic
  variation 1..80: note = internal linkers L1 and L2, 2'-O-Me and PS linkage modifications are present or absent
  source 1..80: mol_type = other RNA

SEQ ID NO: 106 - RNA, 78 nt, synthetic construct; identical to SEQ ID NO: 6
  misc_feature 1..78: note = Synthetic
  variation 1..78: note = internal linkers L1 and L2, 2'-O-Me and PS linkage modifications are present or absent
  source 1..78: mol_type = other RNA

SEQ ID NO: 107 - RNA, 78 nt, synthetic construct; identical to SEQ ID NO: 7
  misc_feature 1..78: note = Synthetic
  variation 1..78: note = internal linkers L1 and L2, 2'-O-Me and PS linkage modifications are present or absent
  source 1..78: mol_type = other RNA

SEQ ID NO: 108 - RNA, 76 nt, synthetic construct; identical to SEQ ID NO: 8
  misc_feature 1..76: note = Synthetic
  variation 1..76: note = internal linkers L1 and L2, 2'-O-Me and PS linkage modifications are present or absent
  source 1..76: mol_type = other RNA

SEQ ID NO: 109 - RNA, 90 nt, synthetic construct; identical to SEQ ID NO: 9
  misc_feature 1..90: note = Synthetic
  variation 1..90: note = 2'-O-Me and PS modifications are present or absent
  source 1..90: mol_type = other RNA

SEQ ID NO: 110 - RNA, 90 nt, synthetic construct; identical to SEQ ID NO: 10
  misc_feature 1..90: note = Synthetic
  variation 1..90: note = 2'-O-Me and PS modifications are present or absent
  source 1..90: mol_type = other RNA

SEQ ID NO: 111 - RNA, 100 nt, synthetic construct; identical to SEQ ID NO: 11
  misc_feature 1..100: note = Synthetic
  variation 1..100: note = 2'-O-Me and PS modifications are present or absent
  source 1..100: mol_type = other RNA

SEQ ID NO: 112 - RNA, 100 nt, synthetic construct; identical to SEQ ID NO: 12
  misc_feature 1..100: note = Synthetic
  variation 1..100: note = 2'-O-Me and PS modifications are present or absent
  source 1..100: mol_type = other RNA

SEQ ID NO: 113 - RNA, 88 nt, synthetic construct; identical to SEQ ID NO: 13
  misc_feature 1..88: note = Synthetic
  variation 1..88: note = 2'-O-Me and PS modifications are present or absent
  source 1..88: mol_type = other RNA

SEQ ID NO: 114 - RNA, 88 nt, synthetic construct; identical to SEQ ID NO: 14
  misc_feature 1..88: note = Synthetic
  variation 1..88: note = 2'-O-Me and PS modifications are present or absent
  source 1..88: mol_type = other RNA

SEQ ID NO: 115-119 - skipped (5 entries)

SEQ ID NO: 120 - RNA, 86 nt, synthetic construct; identical to SEQ ID NO: 20
  misc_feature 1..86: note = Synthetic
  variation 1..86: note = internal linker L2, 2'-O-Me and PS linkage modifications are present or absent
  source 1..86: mol_type = other RNA

SEQ ID NO: 121 - RNA, 85 nt, synthetic construct; identical to SEQ ID NO: 21
  misc_feature 1..85: note = Synthetic
  variation 1..85: note = internal linker L2, 2'-O-Me and PS linkage modifications are present or absent
  source 1..85: mol_type = other RNA

SEQ ID NO: 122 - RNA, 84 nt, synthetic construct; identical to SEQ ID NO: 22
  misc_feature 1..84: note = Synthetic
  variation 1..84: note = internal linker L2, 2'-O-Me and PS linkage modifications are present or absent
  source 1..84: mol_type = other RNA

SEQ ID NO: 123 - RNA, 83 nt, synthetic construct; identical to SEQ ID NO: 23
  misc_feature 1..83: note = Synthetic
  variation 1..83: note = internal linker L2, 2'-O-Me and PS linkage modifications are present or absent
  source 1..83: mol_type = other RNA

SEQ ID NO: 124 - RNA, 81 nt, synthetic construct; identical to SEQ ID NO: 24
  misc_feature 1..81: note = Synthetic
  variation 1..81: note = internal linker L2, 2'-O-Me and PS linkage modifications are present or absent
  source 1..81: mol_type = other RNA

SEQ ID NO: 125 - RNA, 82 nt, synthetic construct; identical to SEQ ID NO: 25
  misc_feature 1..82: note = Synthetic
  variation 1..82: note = internal linker L2, 2'-O-Me and PS linkage modifications are present or absent
  source 1..82: mol_type = other RNA

SEQ ID NO: 126 - RNA, 79 nt, synthetic construct; identical to SEQ ID NO: 26
  misc_feature 1..79: note = Synthetic
  variation 1..79: note = internal linker L2, 2'-O-Me and PS linkage modifications are present or absent
  source 1..79: mol_type = other RNA

SEQ ID NO: 127 - RNA, 86 nt, synthetic construct; identical to SEQ ID NO: 20
  misc_feature 1..86: note = Synthetic
  variation 1..86: note = internal linker L1, 2'-O-Me and PS linkage modifications are present or absent
  source 1..86: mol_type = other RNA

SEQ ID NO: 128 - RNA, 85 nt, synthetic construct; identical to SEQ ID NO: 21
  misc_feature 1..85: note = Synthetic
  variation 1..85: note = internal linker L1, 2'-O-Me and PS linkage modifications are present or absent
  source 1..85: mol_type = other RNA

SEQ ID NO: 129 - RNA, 84 nt, synthetic construct; identical to SEQ ID NO: 22
  misc_feature 1..84: note = Synthetic
  variation 1..84: note = internal linker L1, 2'-O-Me and PS linkage modifications are present or absent
  source 1..84: mol_type = other RNA

SEQ ID NO: 130 - RNA, 83 nt, synthetic construct; identical to SEQ ID NO: 23
  misc_feature 1..83: note = Synthetic
  variation 1..83: note = internal linker L1, 2'-O-Me and PS linkage modifications are present or absent
  source 1..83: mol_type = other RNA

SEQ ID NO: 131 - RNA, 82 nt, synthetic construct; identical to SEQ ID NO: 25
  misc_feature 1..82: note = Synthetic
  variation 1..82: note = internal linker L1, 2'-O-Me and PS linkage modifications are present or absent
  source 1..82: mol_type = other RNA

SEQ ID NO: 132 - RNA, 81 nt, synthetic construct; identical to SEQ ID NO: 24
  misc_feature 1..81: note = Synthetic
  variation 1..81: note = internal linker L1, 2'-O-Me and PS linkage modifications are present or absent
  source 1..81: mol_type = other RNA

SEQ ID NO: 133 - RNA, 79 nt, synthetic construct; identical to SEQ ID NO: 26
  misc_feature 1..79: note = Synthetic
  variation 1..79: note = internal linker L1, 2'-O-Me and PS linkage modifications are present or absent
  source 1..79: mol_type = other RNA

SEQ ID NO: 134 - RNA, 87 nt, synthetic construct; identical to SEQ ID NO: 34
  misc_feature 1..87: note = Synthetic
  variation 1..87: note = internal L4, 2'-O-Me and PS linkage modifications are present or absent
  source 1..87: mol_type = other RNA

SEQ ID NO: 135 - RNA, 87 nt, synthetic construct; identical to SEQ ID NO: 34
  misc_feature 1..87: note = Synthetic
  variation 1..87: note = internal linker L2, 2'-O-Me and PS linkage modifications are present or absent
  source 1..87: mol_type = other RNA

SEQ ID NO: 136 - RNA, 87 nt, synthetic construct; identical to SEQ ID NO: 34
  misc_feature 1..87: note = Synthetic
  variation 1..87: note = internal linker L1, 2'-O-Me and PS linkage modifications are present or absent
  source 1..87: mol_type = other RNA

SEQ ID NO: 137 - RNA, 88 nt, synthetic construct; identical to SEQ ID NO: 37
  misc_feature 1..88: note = Synthetic
  variation 1..88: note = internal linker L2, 2'-O-Me and PS linkage modifications are present or absent
  source 1..88: mol_type = other RNA

SEQ ID NO: 138 - RNA, 87 nt, synthetic construct; identical to SEQ ID NO: 38
  misc_feature 1..87: note = Synthetic
  variation 1..87: note = internal linker L2, 2'-O-Me and PS linkage modifications are present or absent
  source 1..87: mol_type = other RNA

SEQ ID NO: 139 - RNA, 87 nt, synthetic construct; identical to SEQ ID NO: 39
  misc_feature 1..87: note = Synthetic
  variation 1..87: note = internal linker L2, 2'-O-Me and PS linkage modifications are present or absent
  source 1..87: mol_type = other RNA

SEQ ID NO: 140 - RNA, 86 nt, synthetic construct; identical to SEQ ID NO: 40
  misc_feature 1..86: note = Synthetic
  variation 1..86: note = internal linker L2, 2'-O-Me and PS linkage modifications are present or absent
  source 1..86: mol_type = other RNA

SEQ ID NO: 141 - RNA, 88 nt, synthetic construct; identical to SEQ ID NO: 37
  misc_feature 1..88: note = Synthetic
  variation 1..88: note = internal linker L1, 2'-O-Me and PS linkage modifications are present or absent
  source 1..88: mol_type = other RNA

SEQ ID NO: 142 - RNA, 87 nt, synthetic construct; identical to SEQ ID NO: 38
  misc_feature 1..87: note = Synthetic
  variation 1..87: note = internal linker L1, 2'-O-Me and PS linkage modifications are present or absent
  source 1..87: mol_type = other RNA

SEQ ID NO: 143 - RNA, 87 nt, synthetic construct; identical to SEQ ID NO: 39
  misc_feature 1..87: note = Synthetic
  variation 1..87: note = internal linker L1, 2'-O-Me and PS linkage modifications are present or absent
  source 1..87: mol_type = other RNA

SEQ ID NO: 144 - RNA, 86 nt, synthetic construct; identical to SEQ ID NO: 40
  misc_feature 1..86: note = Synthetic
  variation 1..86: note = internal linker L1, 2'-O-Me and PS linkage modifications are present or absent
  source 1..86: mol_type = other RNA

SEQ ID NO: 145 - RNA, 83 nt, synthetic construct; identical to SEQ ID NO: 45
  misc_feature 1..83: note = Synthetic
  variation 1..83: note = internal linkers L1 and L2, 2'-O-Me and PS linkage modifications are present or absent
  source 1..83: mol_type = other RNA

SEQ ID NO: 146 - RNA, 83 nt, synthetic construct; identical to SEQ ID NO: 45
  misc_feature 1..83: note = Synthetic
  variation 1..83: note = internal linker L1, 2'-O-Me and PS linkage modifications are present or absent
  source 1..83: mol_type = other RNA

SEQ ID NO: 147 - RNA, 83 nt, synthetic construct; identical to SEQ ID NO: 47
  misc_feature 1..83: note = Synthetic
  variation 1..83: note = internal linkers L1 and L2, 2'-O-Me and PS linkage modifications are present or absent
  source 1..83: mol_type = other RNA

SEQ ID NO: 148 - RNA, 83 nt, synthetic construct; identical to SEQ ID NO: 47
  misc_feature 1..83: note = Synthetic
  variation 1..83: note = internal linker L1, 2'-O-Me and PS linkage modifications are present or absent
  source 1..83: mol_type = other RNA

SEQ ID NO: 149 - RNA, 81 nt, synthetic construct; identical to SEQ ID NO: 49
  misc_feature 1..81: note = Synthetic
  variation 1..81: note = internal linkers L1 and L2, 2'-O-Me and PS linkage modifications are present or absent
  source 1..81: mol_type = other RNA

SEQ ID NO: 150 - RNA, 81 nt, synthetic construct; identical to SEQ ID NO: 49
  misc_feature 1..81: note = Synthetic
  variation 1..81: note = internal linker L1, 2'-O-Me and PS linkage modifications are present or absent
  source 1..81: mol_type = other RNA

SEQ ID NO: 151 - RNA, 82 nt, synthetic construct; identical to SEQ ID NO: 51
  misc_feature 1..82: note = Synthetic
  variation 1..82: note = internal linkers L1 and L2, 2'-O-Me and PS linkage modifications are present or absent
  source 1..82: mol_type = other RNA

SEQ ID NO: 152 - RNA, 82 nt, synthetic construct; identical to SEQ ID NO: 51
  misc_feature 1..82: note = Synthetic
  variation 1..82: note = internal linker L1, 2'-O-Me and PS linkage modifications are present or absent
  source 1..82: mol_type = other RNA

SEQ ID NO: 153 - RNA, 79 nt, synthetic construct; identical to SEQ ID NO: 53
  misc_feature 1..79: note = Synthetic
  variation 1..79: note = internal linkers L1 and L2, 2'-O-Me and PS linkage modifications are present or absent
  source 1..79: mol_type = other RNA

SEQ ID NO: 154 - RNA, 79 nt, synthetic construct; identical to SEQ ID NO: 53
  misc_feature 1..79: note = Synthetic
  variation 1..79: note = internal linker L1, 2'-O-Me and PS linkage modifications are present or absent
  source 1..79: mol_type = other RNA

SEQ ID NO: 155 - RNA, 80 nt, synthetic construct; identical to SEQ ID NO: 55
  misc_feature 1..80: note = Synthetic
  variation 1..80: note = internal linkers L1 and L2, 2'-O-Me and PS linkage modifications are present or absent
  source 1..80: mol_type = other RNA

SEQ ID NO: 156 - RNA, 80 nt, synthetic construct; identical to SEQ ID NO: 55
  misc_feature 1..80: note = Synthetic
  variation 1..80: note = internal linker L1, 2'-O-Me and PS linkage modifications are present or absent
  source 1..80: mol_type = other RNA

SEQ ID NO: 157 - RNA, 77 nt, synthetic construct; identical to SEQ ID NO: 57
  misc_feature 1..77: note = Synthetic
  variation 1..77: note = internal linkers L1 and L2, 2'-O-Me and PS linkage modifications are present or absent
  source 1..77: mol_type = other RNA

SEQ ID NO: 158 - RNA, 77 nt, synthetic construct; identical to SEQ ID NO: 57
  misc_feature 1..77: note = Synthetic
  variation 1..77: note = internal linker L1, 2'-O-Me and PS linkage modifications are present or absent
  source 1..77: mol_type = other RNA

SEQ ID NO: 159 - RNA, 87 nt, synthetic construct; identical to SEQ ID NO: 59
  misc_feature 1..87: note = Synthetic
  variation 1..87: note = internal linker L2, 2'-O-Me and PS linkage modifications are present or absent
  source 1..87: mol_type = other RNA

SEQ ID NO: 160 - RNA, 87 nt, synthetic construct; identical to SEQ ID NO: 59
  misc_feature 1..87: note = Synthetic
  variation 1..87: note = internal linker L1, 2'-O-Me and PS linkage modifications are present or absent
  source 1..87: mol_type = other RNA

SEQ ID NO: 161 - RNA, 85 nt, synthetic construct; identical to SEQ ID NO: 61
  misc_feature 1..85: note = Synthetic
  variation 1..85: note = internal linker L1, 2'-O-Me and PS linkage modifications are present or absent
  source 1..85: mol_type = other RNA

SEQ ID NO: 162 - RNA, 85 nt, synthetic construct; identical to SEQ ID NO: 61
  misc_feature 1..85: note = Synthetic
  variation 1..85: note = internal linker L2, 2'-O-Me and PS linkage modifications are present or absent
  source 1..85: mol_type = other RNA

SEQ ID NO: 163 - RNA, 90 nt, synthetic construct; identical to SEQ ID NO: 10
  misc_feature 1..90: note = Synthetic
  variation 1..90: note = 2'-O-Me and PS modifications are present or absent
  source 1..90: mol_type = other RNA

SEQ ID NO: 164 - RNA, 90 nt, synthetic construct; identical to SEQ ID NO: 10
  misc_feature 1..90: note = Synthetic
  variation 1..90: note = 2'-O-Me and PS modifications are present or absent
  source 1..90: mol_type = other RNA

SEQ ID NO: 165 - RNA, 86 nt, synthetic construct; identical to SEQ ID NO: 65
  misc_feature 1..86: note = Synthetic
  variation 1..86: note = internal linker L1, 2'-O-Me and PS linkage modifications are present or absent
  source 1..86: mol_type = other RNA

SEQ ID NO: 166 - RNA, 86 nt, synthetic construct; identical to SEQ ID NO: 66
  misc_feature 1..86: note = Synthetic
  variation 1..86: note = internal linker L1, 2'-O-Me and PS linkage modifications are present or absent
  source 1..86: mol_type = other RNA

SEQ ID NO: 167 - RNA, 80 nt, synthetic construct; identical to SEQ ID NO: 5
  misc_feature 1..80: note = Synthetic
  variation 1..80: note = internal linker L1, 2'-O-Me and PS linkage modifications are present or absent
  source 1..80: mol_type = other RNA

SEQ ID NO: 168 - RNA, 82 nt, synthetic construct; identical to SEQ ID NO: 68
  misc_feature 1..82: note = Synthetic
  variation 1..82: note = internal linker L1, 2'-O-Me and PS linkage modifications are present or absent
  source 1..82: mol_type = other RNA

SEQ ID NO: 169 - RNA, 86 nt, synthetic construct; identical to SEQ ID NO: 65
  misc_feature 1..86: note = Synthetic
  variation 1..86: note = internal linker L2, 2'-O-Me and PS linkage modifications are present or absent
  source 1..86: mol_type = other RNA

SEQ ID NO: 170 - RNA, 88 nt, synthetic construct; identical to SEQ ID NO: 70
  misc_feature 1..88: note = Synthetic
  variation 1..88: note = 2'-O-Me and PS modifications are present or absent
  source 1..88: mol_type = other RNA

SEQ ID NO: 171 - RNA, 88 nt, synthetic construct; identical to SEQ ID NO: 70
  misc_feature 1..88: note = Synthetic
  variation 1..88: note = 2'-O-Me and PS modifications are present or absent
  source 1..88: mol_type = other RNA

SEQ ID NO: 172 - RNA, 84 nt, synthetic construct; identical to SEQ ID NO: 72
  misc_feature 1..84: note = Synthetic
  variation 1..84: note = internal linker L1, 2'-O-Me and PS linkage modifications are present or absent
  source 1..84: mol_type = other RNA

SEQ ID NO: 173 - RNA, 86 nt, synthetic construct; identical to SEQ ID NO: 73
  misc_feature 1..86: note = Synthetic
  variation 1..86: note = internal linker L1, 2'-O-Me and PS linkage modifications are present or absent
  source 1..86: mol_type = other RNA

SEQ ID NO: 174 - RNA, 82 nt, synthetic construct; identical to SEQ ID NO: 74
  misc_feature 1..82: note = Synthetic
  variation 1..82: note = internal linker L1, 2'-O-Me and PS linkage modifications are present or absent
  source 1..82: mol_type = other RNA

SEQ ID NO: 175 - RNA, 76 nt, synthetic construct; identical to SEQ ID NO: 75
  misc_feature 1..76: note = Synthetic
  variation 1..76: note = internal linker L1, 2'-O-Me and PS linkage modifications are present or absent
  source 1..76: mol_type = other RNA

SEQ ID NO: 176 - skipped

SEQ ID NO: 177 - RNA, 75 nt, synthetic construct; identical to SEQ ID NO: 77
  misc_feature 1..75: note = Synthetic
  variation 1..75: note = internal linker L1, dS site, 2'-O-Me and PS linkage modifications are present or absent
  source 1..75: mol_type = other RNA

SEQ ID NO: 178 - RNA, 75 nt, synthetic construct; identical to SEQ ID NO: 77
  misc_feature 1..75: note = Synthetic
  variation 1..75: note = internal linkers L4 and L1, 2'-O-Me and PS linkage modifications are present or absent
  source 1..75: mol_type = other RNA

SEQ ID NO: 179 - RNA, 75 nt, synthetic construct; identical to SEQ ID NO: 77
  misc_feature 1..75: note = Synthetic
  variation 1..75: note = internal linkers L3 and L1, 2'-O-Me and PS linkage modifications are present or absent
  source 1..75: mol_type = other RNA

SEQ ID NO: 180 - RNA, 75 nt, synthetic construct; identical to SEQ ID NO: 77
  misc_feature 1..75: note = Synthetic
  variation 1..75: note = internal linkers L2 and L1, 2'-O-Me and PS linkage modifications are present or absent
  source 1..75: mol_type = other RNA

SEQ ID NO: 181 - RNA, 75 nt, synthetic construct; identical to SEQ ID NO: 77
  misc_feature 1..75: note = Synthetic
  variation 1..75: note = internal linker L1, 2'-O-Me and PS linkage modifications are present or absent
  source 1..75: mol_type = other RNA

SEQ ID NO: 182 - RNA, 79 nt, synthetic construct; identical to SEQ ID NO: 82
  misc_feature 1..79: note = Synthetic
  variation 1..79: note = internal linker L1, 2'-O-Me and PS linkage modifications are present or absent
  source 1..79: mol_type = other RNA

SEQ ID NO: 183 - RNA, 83 nt, synthetic construct; identical to SEQ ID NO: 83
  misc_feature 1..83: note = Synthetic
  variation 1..83: note = internal linker L1, 2'-O-Me and PS linkage modifications are present or absent
  source 1..83: mol_type = other RNA

SEQ ID NO: 184 - RNA, 83 nt, synthetic construct; identical to SEQ ID NO: 83
  misc_feature 1..83: note = Synthetic
  variation 1..83: note = internal linker L1, 2'-O-Me and PS linkage modifications are present or absent
  source 1..83: mol_type = other RNA

SEQ ID NO: 185-199 - skipped (15 entries)

SEQ ID NO: 200 - RNA, 100 nt, synthetic construct
  misc_feature 1..100: note = Synthetic
  source 1..100: mol_type = other RNA
nnnnnnnnnnnnnnnnnnnngttttagagctagaaatagcaagttaaaataaggctagtccgttatcaacttgaaaaagtggcaccgagtcggtgctttt

SEQ ID NO: 201 - RNA, 90 nt, synthetic construct
  misc_feature 1..90: note = Synthetic
  source 1..90: mol_type = other RNA
nnnnnnnnnnnnnnnnnnnngttttagagctagaaatagcaagttaaaataaggctagtccgttatcacgaaagggcaccgagtcggtgc

SEQ ID NO: 202 - RNA, 100 nt, synthetic construct
  misc_feature 1..100: note = Synthetic
  source 1..100: mol_type = other RNA
nnnnnnnnnnnnnnnnnnnngttttagtactctggaaacagaatctactaaaacaaggcaaaatgccgtgtttatctcgtcaacttgttggcgagatttt

SEQ ID NO: 203 - RNA, 113 nt, synthetic construct
  misc_feature 1..113: note = Synthetic
  source 1..113: mol_type = other RNA
nnnnnnnnnnnnnnnnnnnnactggggttcaggaaactgaacctcagtaagcattggctcgtttccaatgttgattgctccgccggtgctccttatttttaagggcgccggca

SEQ ID NO: 204 - RNA, 117 nt, synthetic construct
  misc_feature 1..117: note = Synthetic
  source 1..117: mol_type = other RNA
nnnnnnnnnnnnnnnnnnnngtttttgtactctcaagattcaataatcttgcagaagctacaaagataaggcttcatgccgaaatcaacaccctgtcattttatggcagggtgtttt

SEQ ID NO: 205 - RNA, 97 nt, synthetic construct
  misc_feature 1..97: note = Synthetic
  source 1..97: mol_type = other RNA
nnnnnnnnnnnnnnnnnnnngtttttgtactcgaaagaagctacaaagataaggcttcatgccgaaatcaacaccctgtcattttatggcagggtgt

SEQ ID NO: 206 - RNA, 94 nt, synthetic construct
  misc_feature 1..94: note = Synthetic
  source 1..94: mol_type = other RNA
nnnnnnnnnnnnnnnnnnnngctggggagcctgaaaaggctacctagcaagaccccttcgtggggtcgcattcttcacccccagcagggggttc

SEQ ID NO: 207 - RNA, 93 nt, synthetic construct
  misc_feature 1..93: note = Synthetic
  source 1..93: mol_type = other RNA
nnnnnnnnnnnnnnnnnnnngttttagtccctgaaaagggactaaaataaagagtttgcgggactctgcggggttacaatcccctaaaaccgc

SEQ ID NO: 208 - RNA, 94 nt, synthetic construct
  misc_feature 1..94: note = Synthetic
  source 1..94: mol_type = other RNA
nnnnnnnnnnnnnnnnnnnnngtttcagttgcgccgaaaggcgctctgtaatcattaaaaagtattttgaacggacctctgtttgacacgtctg

SEQ ID NO: 209 - RNA, 45 nt, synthetic construct
  misc_feature 1..45: note = Synthetic
  source 1..45: mol_type = other RNA
taatttctactcttgtagatnnnnnnnnnnnnnnnnnnnnnnnnn

SEQ ID NO: 210 - RNA, 55 nt, synthetic construct
  misc_feature 1..55: note = Synthetic
  source 1..55: mol_type = other RNA
ctggtgcaaatttgcactagcttaaaacnnnnnnnnnnnnnnnnnnnnnnnnnnn

SEQ ID NO: 211 - RNA, 90 nt, synthetic construct
  misc_feature 1..90: note = Synthetic
  variation 1..90: note = internal linkers L1 and L2 are present or absent
  source 1..90: mol_type = other RNA
nnnnnnnnnnnnnnnnnnnngttttagagctatagcaagttaaaataaggcgtccgttatcaacttaagtggcaccgagtcggtgctttt

SEQ ID NO: 212 - RNA, 80 nt, synthetic construct
  misc_feature 1..80: note = Synthetic
  variation 1..80: note = internal linkers L1 and L2 are present or absent
  source 1..80: mol_type = other RNA
nnnnnnnnnnnnnnnnnnnngttttagagctatagcaagttaaaataaggcgtccgttatcacgggcaccgagtcggtgc

SEQ ID NO: 213 - RNA, 80 nt, synthetic construct; identical to SEQ ID NO: 212
  misc_feature 1..80: note = Synthetic
  variation 1..80: note = internal linkers L1 and L2, 2'-O-Me and PS linkage modifications are present or absent
  source 1..80: mol_type = other RNA

SEQ ID NO: 214 - RNA, 78 nt, synthetic construct
  misc_feature 1..78: note = Synthetic
  variation 1..78: note = internal linkers L1 and L2, 2'-O-Me and PS linkage modifications are present or absent
  source 1..78: mol_type = other RNA
nnnnnnnnnnnnnnnnnnnngttttagagctatagcaagttaaaataaggcgtccgttatcaggcaccgagtcggtgc

SEQ ID NO: 215 - RNA, 78 nt, synthetic construct
  misc_feature 1..78: note = Synthetic
  variation 1..78: note = internal linkers L1 and L2, 2'-O-Me and PS linkage modifications are present or absent
  source 1..78: mol_type = other RNA
nnnnnnnnnnnnnnnnnnnngttttagagctagcaagttaaaataaggcgtccgttatcacgggcaccgagtcggtgc

SEQ ID NO: 216 - RNA, 76 nt, synthetic construct
  misc_feature 1..76: note = Synthetic
  variation 1..76: note = internal linkers L1 and L2, 2'-O-Me and PS linkage modifications are present or absent
  source 1..76: mol_type = other RNA
nnnnnnnnnnnnnnnnnnnngttttagagctagcaagttaaaataaggcgtccgttatcaggcaccgagtcggtgc

SEQ ID NO: 217 - RNA, 76 nt, synthetic construct; identical to SEQ ID NO: 216
  misc_feature 1..76: note = Synthetic
  variation 1..76: note = internal linkers L1 and L2, 2'-O-Me and PS linkage modifications are present or absent
  source 1..76: mol_type = other RNA

SEQ ID NO: 218 - RNA, 76 nt, synthetic construct; identical to SEQ ID NO: 216
  misc_feature 1..76: note = Synthetic
  variation 1..76: note = internal linkers L1 and L2, 2'-O-Me and PS linkage modifications are present or absent
  source 1..76: mol_type = other RNA

SEQ ID NO: 219 - RNA, 80 nt, synthetic construct; identical to SEQ ID NO: 212
  misc_feature 1..80: note = Synthetic
  variation 1..80: note = internal linkers L1 and L2, 2'-O-Me and PS linkage modifications are present or absent
  source 1..80: mol_type = other RNA

SEQ ID NO: 220 - RNA, 78 nt, synthetic construct; identical to SEQ ID NO: 214
  misc_feature 1..78: note = Synthetic
  variation 1..78: note = internal linkers L1 and L2, 2'-O-Me and PS linkage modifications are present or absent
  source 1..78: mol_type = other RNA

SEQ ID NO: 221 - RNA, 78 nt, synthetic construct; identical to SEQ ID NO: 215
  misc_feature 1..78: note = Synthetic
  variation 1..78: note = internal linkers L1 and L2, 2'-O-Me and PS linkage modifications are present or absent
  source 1..78: mol_type = other RNA

SEQ ID NO: 222 - RNA, 76 nt, synthetic construct; identical to SEQ ID NO: 216
  misc_feature 1..76: note = Synthetic
  variation 1..76: note = internal linkers L1 and L2, 2'-O-Me and PS linkage modifications are present or absent
  source 1..76: mol_type = other RNA

SEQ ID NO: 223 - RNA, 75 nt, synthetic construct
  misc_feature 1..75: note = Synthetic
  variation 1..75: note = internal linker L1, dS site, 2'-O-Me and PS linkage modifications are present or absent
  source 1..75: mol_type = other RNA
nnnnnnnnnnnnnnnnnnnngttttagaaagttaaaataaggctagtccgttatcacgggcaccgagtcggtgct

SEQ ID NO: 224 - RNA, 75 nt, synthetic construct; identical to SEQ ID NO: 223
  misc_feature 1..75: note = Synthetic
  variation 1..75: note = internal linkers L1 and L4, 2'-O-Me and PS linkage modifications are present or absent
  source 1..75: mol_type = other RNA

SEQ ID NO: 225 - RNA, 75 nt, synthetic construct; identical to SEQ ID NO: 223
  misc_feature 1..75: note = Synthetic
  variation 1..75: note = internal linkers L1 and L3, 2'-O-Me and PS linkage modifications are present or absent
  source 1..75: mol_type = other RNA

SEQ ID NO: 226 - RNA, 75 nt, synthetic construct; identical to SEQ ID NO: 223
  misc_feature 1..75: note = Synthetic
  variation 1..75: note = internal linkers L1 and L2, 2'-O-Me and PS linkage modifications are present or absent
  source 1..75: mol_type = other RNA

SEQ ID NO: 227 - RNA, 75 nt, synthetic construct; identical to SEQ ID NO: 223
  misc_feature 1..75: note = Synthetic
  variation 1..75: note = internal linker L1, 2'-O-Me and PS linkage modifications are present or absent
  source 1..75: mol_type = other RNA

SEQ ID NO: 228 - RNA, 79 nt, synthetic construct
  misc_feature 1..79: note = Synthetic
  variation 1..79: note = internal linker L1, 2'-O-Me and PS linkage modifications are present or absent
  source 1..79: mol_type = other RNA
nnnnnnnnnnnnnnnnnnnngttttagagcgcaagttaaaataaggctagtccgttatcacgggcaccgagtcggtgct

SEQ ID NO: 229 - RNA, 83 nt, synthetic construct
  misc_feature 1..83: note = Synthetic
  variation 1..83: note = internal linker L1, 2'-O-Me and PS linkage modifications are present or absent
  source 1..83: mol_type = other RNA
nnnnnnnnnnnnnnnnnnnngttttagagctatagcaagttaaaataaggctagtccgttatcacgggcaccgagtcggtgct

SEQ ID NO: 230 - RNA, 83 nt, synthetic construct; identical to SEQ ID NO: 229
  misc_feature 1..83: note = Synthetic
  variation 1..83: note = internal linker L1, 2'-O-Me and PS linkage modifications are present or absent
  source 1..83: mol_type = other RNA

SEQ ID NO: 231-300 - skipped (70 entries)

SEQ ID NO: 301 - protein, 1379 aa, synthetic construct
  REGION 1..1379: note = Synthetic
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGSPKKKRKV

SEQ ID NO: 302 - protein, 1074 aa, synthetic construct
  REGION 1..1074: note = Synthetic
MALEAPKKKRKVGSDYKDDDDKKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 303 - protein, 1091 aa, synthetic construct
  REGION 1..1091: note = Synthetic
MKYHVGIDVGTFSVGLAAIEVDDAGMPIKTLSLVSHIHDSGLDPDKIKSAVTRLASSGIARRTRRLYRRKRRRLQQLDKFIQRQGWPVIELEDYSDPLYPWKVRAELAASYIADEKERGEKLSVALRHIARHRGWRNPYAKVSSLYLPDEPSDAFKAIREEIKRASGQPVPETATVGQMVTLCELGTLKLRGEGGVLSARLQQSDHAREIQEICRMQEIGQELYRKIIDVVFAAESPKGSASSRVGKDPLQPGKNRALKASDAFQRYRIAALIGNLRVRVDGEKRILSVEEKNLVFDHLVNLAPKKEPEWVTIAEILGIDRGQLIGTATMTDDGERAGARPPTHDTNRSIVNSRIAPLVDWWKTASALEQHAMVKALSNAEVDDFDSPEGAKVQAFFADLDDDVHAKLDSLHLPVGRAAYSEDTLVRLTRRMLADGVDLYTARLQEFGIEPSWTPPAPRIGEPVGNPAVDRVLKTVSRWLESATKTWGAPERVIIEHVREGFVTEKRAREMDGDMRRRAARNAKLFQEMQEKLNVQGKPSRADLWRYQSVQRQNCQCAYCGSPITFSNSEMDHIVPRAGQGSTNTRENLVAVCHRCNQSKGNTPFAIWAKNTSIEGVSVKEAVERTRHWVTDTGMRSTDFKKFTKAVVERFQRATMDEEIDARSMESVAWMANELRSRVAQHFASHGTTVRVYRGSLTAEARRASGISGKLEFLDGVGKSRLDRRHHAIDAAVIAFTSDYVAETLAVRSNLKQSQAHRQEAPQWREFTGKDAEHRAAWRVWCQKMEKLSALLTEDLRDDRVVVMSNVRLRLGNGSAHEETIGKLSKVKLGSQLSVSDIDKASSEALWCALTREPDFDPKDGLPANPERHIRVNGTHVYAGDNIGLFPVSAGSIALRGGYAELGSSFHHARVYKITSGKKPAFAMLRVYTIDLLPYRNQDLFSVELKPQTMSMRQAEKKLRDALATGNAEYLGWLVVDDELVVDTSKIATDQVKAVEAELGTIRRWRVDGFFGDTRLRLRPLQMSKEGIKKESAPELSKIIDRPGWLPAVNKLFSEGNVTVVRRDSLGRVRLESTAHLPVTWKVQPKKKRKV

SEQ ID NO: 304 - protein, 1121 aa, synthetic construct
  REGION 1..1121: note = Synthetic
MSDLVLGLDIGIGSVGVGILNKVTGEIIHKNSRIFPAAQAENNLVRRTNRQGRRLTRRKKHRIVRLNRLFEESGLITDFTKISINLNPYQLRVKGLTDELSNEELFIALKNMVKHRGISYLDDASDDGNSSVGDYAQIVKENSKQLETKTPGQIQLERYQTYGQLRGDFTVEKDGKKHRLINVFPTSAYRSEALRILQTQQEFNPQITDEFINRYLEILTGKRKYYHGPGNEKSRTDYGRYRTSGETLDNIFGILIGKCTFYPDEFRAAKASYTAQEFNLLNDLNNLTVPTETKKLSKEQKNQIINYVKNEKAMGPAKLFKYIAKLLSCDVADIKEYRIDKSGKAEIHTFEAYRKMKTLETLDIEQMDRETLDKLAYVLTLNTEREGIQEALEHEFADGSFSQKQVDELVQFRKANSSIFGKGWHNFSVKLMMELIPELYETSEEQMTILTRLGKQKTTSSSNKTKYIDEKLLTEEIYNPVVAKSVRQAIKIVNAAIKEYGDFDNIVIEMARETNEDDEKKAIQKIQKANKDEKDAAMLKAANQYNGKAELPHSVFHGHKQLATKIRLWHQQGERCLYTGKTISIHDLINNSNQFEVDHILPLSITFDDSLANKVLVYATANQEKGQRTPYQALDSMDDAWSFRELKAFVRESKTLSNKKKEYLLTEEDISKFDVRKKFIERNLVDTRYASRVVLNALQEHFRAHKIDTKVSVVRGQFTSQLRRHWGIEKTRDTYHHHAVDALIIAASIQLNLWKKQKNTLVSYSEDQLLDIETGELISDDEYKESVFKAPYQHFVDTLKSKEFEDSILFSYQVDSKFNRKISDATIYATRQAKVGKDKADETYVLGKIKDIYTQDGYDAFMKIYKKDKSKFLMYRHDPQTFEKVIEPILENYPNKQINEKGKEVPVNPFLKYKEEHGYIRKYSKKGNGPEIKSLKYYDSKLGNHIDITPKDSNNKVVLQSVSPWRADVYFNKTTGKYEILGLKYADLQFEKGTGTYKISQEKYNDIKKKEGVDSDSEFKFTLYKNDLLLVKDTETKEQQLFRFLSRTMPKQKHYVELKPYDKQKFEGGEALIKVLGNVANSGQCKKGLGKSNISIYKVRTDVLGNQHIIKNEGDKPKLDF

SEQ ID NO: 305 - skipped

SEQ ID NO: 306 - protein, 1155 aa, synthetic construct
  REGION 1..1155: note = Synthetic
MSLQLIIKGVEYGRGADPTMGGSEVGTVPVTWRLGVDVGERSIGLAAVSYEEDKPKEILAAVSWIHDGGVGDERSGASRLALRGMARRARRLRRFRRARLRDLDMLLSELGWTPLPDKNVSPVDAWLARKRLAEEYVVDETERRRLLGYAVSHMARHRGWRNPWTTIKDLKNLPQPSDSWERTRESLEARYSVSLEPGTVGQWAGYLLQRAPGIRLNPTQQSAGRRAELSNATAFETRLRQEDVLWELRCIADVQGLPEDVVSNVIDAVFCQKRPSVPAERIGRDPLDPSQLRASRACLEFQEYRIVAAVANLRIRDGSGSRPLSLEERNAVIEALLAQTERSLTWSDIALEILKLPNESDLTSVPEEDGPSSLAYSQFAPFDETSARIAEFIAKNRRKIPTFAQWWQEQDRTSRSDLVAADNSIAGEEEQELLVHLPDAELEALEGLALPSGRVAYSRLTLSGLTRVMRDDGVDVHNARKTCFGVDDNWRPPLPALHEATGHPVVDRNLAILRKFLSSATMRWGPPQSIVVELARGASESRERQAEEEAARRAHRKANDRIRAELRASGLSDPSPADLVRARLLELYDCHCMYCGAPISWENSELDHIVPRTDGGSNRHENLAITCGACNKEKGRRPFASWAETSNRVQLRDVIDRVQKLKYSGNMYWTRDEFSRYKKSVVARLKRRTSDPEVIQSIESTGYAAVALRDRLLSYGEKNGVAQVAVFRGGVTAEARRWLDISIERLFSRVAIFAQSTSTKRLDRRHHAVDAVVLTTLTPGVAKTLADARSRRVSAEFWRRPSDVNRHSTEEPQSPAYRQWKESCSGLGDLLISTAARDSIAVAAPLRLRPTGALHEETLRAFSEHTVGAAWKGAELRRIVEPEVYAAFLALTDPGGRFLKVSPSEDVLPADENRHIVLSDRVLGPRDRVKLFPDDRGSIRVRGGAAYIASFHHARVFRWGSSHSPSFALLRVSLADLAVAGLLRDGVDVFTAELPPWTPAWRYASIALVKAVESGDAKQVGWLVPGDELDFGPEGVTTAAGDLSMFLKYFPERHWVVTGFEDDKRINLKPAFLSAEQAEVLRTERSDRPDTLTEAGEILAQFFPRCWRATVAKVLCHPGLTVIRRTALGQPRWRRGHLPYSWRPWSADPWSGGTP

SEQ ID NO: 307 - protein, 984 aa, synthetic construct
  REGION 1..984: note = Synthetic
MARILAFDIGISSIGWAFSENDELKDCGVRIFTKAENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGDHSLSQDDLNEIAKDITLIKDEIKLKKALAKYDLNQNQIDSLSKLEFKDHLNISFKALKLITPLMLEGKKYDEACNELNLKVAINEDKKDFLPAFNETYYKDEVTNPVVLRAIKEYRKVLNALLKKYGKVHKINIELAREVGKNYSQRAKIEKEQNENYKAKKDAELECEKLGLKINSKNILKLRLFKEQKEFCAYSGEKIKISDLQDEKMLEIDHIYPYSRSFDDSYMNKVLVFTKQNQEKLNQTPFEAFGNDSAKWQKIEVLAKNLPTKKQKRILDKNYKDKEQKDFKDRNLNDTRYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFKK

SEQ ID NO: 308 - protein, 1629 aa, synthetic construct
  REGION 1..1629: note = Synthetic
MNFKILPIAIDLGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLHKYNNTGRFDDDNQLLTYCNHKPRQKRYQLLNDLAGVLQVSPNFLKDKIGSDDDLFISKWLVEHIRGFKKACEDSLKIQKDNRGLLNHKINIARNTKGKCEKEIFNLICKIEGSEDKKGNYKHGLAYELGVLLFGEPNEASKPEFDRKIKKFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGKSLKDRRKKALERISPENIFKDKNNRIKEFAKGISAYSGANLTDGDFDGAKEELDHIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEKVDSDIQAYAKGDKPQASYSHLIDAMLAFCIAADEHRNDGSIGLEIDKNYSLYPLDKNTGEVFTKDIFSQIKITDNEFSDKKLVRKKAIEGFNTHRQMTRDGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSERVKIKSIDDVKQVLDKDSNFIIGKITLPFKKEWQRLYREWQNTTIKDDYEFLKSFFNVKSITKLHKKVRKDFSLPISTNEGKFLVKRKTWDNNFIYQILNDSDSRVDGTKPFIPAFDISKNEIVEAIIDSFTSKNIFWLPKNIELQKVDNKNIFAIDTSKWFEVETPSDLRDIGVATIQYKIDNNSRPKVRVKLDYVIDDDSKINYFTNHSLLKSRYPDKVLEILKQSTTIEFESSGFNKTIKEMLGMTLAGIYNETSNN

SEQ ID NO: 309 - protein, 1307 aa, synthetic construct
  REGION 1..1307: note = Synthetic
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 310 - protein, 1082 aa, synthetic construct
  REGION 1..1082: note = Synthetic
MAAFKPNPINYILGLDIGIASVGWAMVEIDEEENPIRLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVANNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSSELQDEIGTAFSLFKTDEDITGRLKDRVQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPRSKKQRILLQKFDEDGFKECNLNDTRYVNRFLCQFVADHILLTGKGKRRVFASNGQITNLLRGFWGLRKVRAENDRHHALDAVVVACSTVAMQQKITRFVRYKEMNAFDGKTIDKETGKVLHQKTHFPQPWEFFAQEVMIRVFGKPDGKPEFEEADTPEKLRTLLAEKLSSRPEAVHEYVTPLFVSRAPNRKMSGAHKDTLRSAKRFVKHNEKISVKRVWLTEIKLADLENMVNYKNGREIELYEALKARLEAYGGNAKQAFDPKDNPFYKKGGQLVKAVRVEKTQESGVLLNKKNAYTIADNGDMVRVDVFCKVDKKGKNQYFIVPIYAWQVAENILPDIDCKGYRIDDSYTFCFSLHKYDLIAFQKDEKSKVEFAYYINCDSSNGRFYLAWHDKGSKEQQFRISTQNLVLIQKYQVNELGKEIRPCRLKKRPPVR

SEQ ID NO: 311 - protein, 1173 aa, synthetic construct
  REGION 1..1173: note = Synthetic
MRPIEPWILGLDIGTDSLGWAVFSCEEKGPPTAKELLGGGVRLFDSGRDAKDHTSRQAERGAFRRARRQTRTWPWRRDRLIALFQAAGLTPPAAETRQIALALRREAVSRPLAPDALWAALLHLAHHRGFRSNRIDKRERAAAKALAKAKPAKATAKATAPAKEADDEAGFWEGAEAALRQRMAASGAPTVGALLADDLDRGQPVRMRYNQSDRDGVVAPTRALIAEELAEIVARQSSAYPGLDWPAVTRLVLDQRPLRSKGAGPCAFLPGEDRALRALPTVQDFIIRQTLANLRLPSTSADEPRPLTDEEHAKALALLSTARFVEWPALRRALGLKRGVKFTAETERNGAKQAARGTAGNLTEAILAPLIPGWSGWDLDRKDRVFSDLWAARQDRSALLALIGDPRGPTRVTEDETAEAVADAIQIVLPTGRASLSAKAARAIAQAMAPGIGYDEAVTLALGLHHSHRPRQERLARLPYYAAALPDVGLDGDPVGPPPAEDDGAAAEAYYGRIGNISVHIALNETRKIVNALLHRHGPILRLVMVETTRELKAGADERKRMIAEQAERERENAEIDVELRKSDRWMANARERRQRVRLARRQNNLCPYTSTPIGHADLLGDAYDIDHVIPLARGGRDSLDNMVLCQSDANKTKGDKTPWEAFHDKPGWIAQRDDFLARLDPQTAKALAWRFADDAGERVARKSAEDEDQGFLPRQLTDTGYIARVALRYLSLVTNEPNAVVATNGRLTGLLRLAWDITPGPAPRDLLPTPRDALRDDTAARRFLDGLTPPPLAKAVEGAVQARLAALGRSRVADAGLADALGLTLASLGGGGKNRADHRHHFIDAAMIAVTTRGLINQINQASGAGRILDLRKWPRTNFEPPYPTFRAEVMKQWDHIHPSIRPAHRDGGSLHAATVFGVRNRPDARVLVQRKPVEKLFLDANAKPLPADKIAEIIDGFASPRMAKRFKALLARYQAAHPEVPPALAALAVARDPAFGPRGMTANTVIAGRSDGDGEDAGLITPFRANPKAAVRTMGNAVYEVWEIQVKGRPRWTHRVLTRFDRTQPAPPPPPENARLVMRLRRGDLVYWPLESGDRLFLVKKMAVDGRLALWPARLATGKATALYAQLSCPNINLNGDQGYCVQSAEGIRKEKIRTTSCTALGRLRLSKKAT

SEQ ID NO: 312 - protein, 1147 aa, synthetic construct
  REGION 1..1147: note = Synthetic
MLERHPQRYRLGLDLGSNSLGWFVTNLQQRGDRFEPVGLGPGGVRIFPDGRDPQSKASNAVDRRMARGARKRRDRFVQRRSQLMDALVRHGLMPEEAAARKALAGLDPYQLRARALVDRLPAHHVGRALFHLNQRRGFLSNRKTDNKKNSEDGAIKQAASRLRDSMASQGAETLGEFFAGRRHSDTYAERQTAIRAELQRIGKDHLTGNARKKAWAKVRKRLFGDDVLDPAMAPEGVRARAIITGTKASYDFYPTRDLLLQEFHAIWRAQAPHHSTMTESACREIERIIFYQRPLKDPIVGKCSLDPATRPFKEDPEGYRAPWAHPLAQRFRILSEARNLEIRETGRGSRKLTKAQSDVVALALLGSKEVKFDKLRTLLKLPAESKFNLESDRRAALDGDATAARLSDKNGFGKAWRGFPLERQVAIVERLMTVEVEAELVDWLEQECGLGPEAAARVANTSLPEGHCRLGLRAIKKIVPVMQNEVGDDGVSGAGYYEAAKRIGYDHAKLPTGGDLDYLPYYGKWLADAVVGSGDARDGKERQFGQFPNPTVHIGLGQLRRLVNELIRAYGPPTEISIEFTRALRLSEDQKAQVQREQRKNQDRNRARAAELEQLGFPANPRNLLKMRLWEELNLRDPLDRKCVYTGEQISIERLLSEEVDIDHILPVAMTLDDSAANKIVCMRYANRHKRKQTPFEAFGSSPLVQGRRYAWDDIATRAAALPRNKRWRFDADAREQFDKRGGFLARQLNETGWLARLAKEYLGAVTDPNRIWVVPGRLTGLLRGKWGLNALLPDHNYAGVQDKAEAFLASTDDMEFSGVKNRADHRHHAIDGLVAALTDRSLLWKMANAYDDEREKFVVELPWPTMRDDLKAALDKMVVSHKPDHGVQGKLHEDSAYGLLDRPEPVEDDDREPANLVYRKAVETLSENEIGRIRDRRLRDLVRDHVDAAKRNGVALADALRQLTEPSDNPHFRHGLRHVRLLKKEKTDYLVPVVDRATGRPYKAYSAGENFCVEVFETADGKWSGEAVRRFDANRSNCGPKTPHVPRWRTESAGARLVMRIHKGDLIRLEHEGRTRIMVVHRLDAAAGRFKLAAHNETGNLDKRHATDNDIDPFRWLMASYGTLKKMAAVPVRVDELGRVWRIDPR

SEQ ID NO: 313 - protein, 1101 aa, synthetic construct
  REGION 1..1101: note = Synthetic
MWYASLMSAHHLRVGIDVGTHSVGLATLRVDDHGTPIELLSALSHIHDSGVGKEGKKDHDTRKKLSGIARRARRLLHHRRTQLQQLDEVLRDLGFPIPTPGEFLDLNEQTDPYRVWRVRARLVEEKLPEELRGPAISMAVRHIARHRGWRNPYSKVESLLSPAEESPFMKALRERILATTGEVLDDGITPGQAMAQVALTHNISMRGPEGILGKLHQSDNANEIRKICARQGVSPDVCKQLLRAVFKADSPRGSAVSRVAPDPLPGQGSFRRAPKCDPEFQRFRIISIVANLRISETKGENRPLTADERRHVVTFLTEDSQADLTWVDVAEKLGVHRRDLRGTAVHTDDGERSAARPPIDATDRIMRQTKISSLKTWWEEADSEQRGAMIRYLYEDPTDSECAEIIAELPEEDQAKLDSLHLPAGRAAYSRESLTALSDHMLATTDDLHEARKRLFGVDDSWAPPAEAINAPVGNPSVDRTLKIVGRYLSAVESMWGTPEVIHVEHVRDGFTSERMADERDKANRRRYNDNQEAMKKIQRDYGKEGYISRGDIVRLDALELQGCACLYCGTTIGYHTCQLDHIVPQAGPGSNNRRGNLVAVCERCNRSKSNTPFAVWAQKCGIPHVGVKEAIGRVRGWRKQTPNTSSEDLTRLKKEVIARLRRTQEDPEIDERSMESVAWMANELHHRIAAAYPETTVMVYRGSITAAARKAAGIDSRINLIGEKGRKDRIDRRHHAVDASVVALMEASVAKTLAERSSLRGEQRLTGKEQTWKQYTGSTVGAREHFEMWRGHMLHLTELFNERLAEDKVYVTQNIRLRLSDGNAHTVNPSKLVSHRLGDGLTVQQIDRACTPALWCALTREKDFDEKNGLPAREDRAIRVHGHEIKSSDYIQVFSKRKKTDSDRDETPFGAIAVRGGFVEIGPSIHHARIYRVEGKKPVYAMLRVFTHDLLSQRHGDLFSAVIPPQSISMRCAEPKLRKAITTGNATYLGWVVVGDELEINVDSFTKYAIGRFLEDFPNTTRWRICGYDTNSKLTLKPIVLAAEGLENPSSAVNEIVELKGWRVAINVLTKVHPTVVRRDALGRPRYSSRSNLPTSWTIE

SEQ ID NO: 314-320 - skipped (7 entries)

SEQ ID NO: 321 - RNA, 4140 nt, synthetic construct
  misc_feature 1..4140: note = Synthetic
  source 1..4140: mol_type = other RNA
atggacaagaagtacagcatcggactggacatcggaacaaacagcgtcggatgggcagtcatcacagacgaatacaaggtcccgagcaagaagttcaaggtcctgggaaacacagacagacacagcatcaagaagaacctgatcggagcactgctgttcgacagcggagaaacagcagaagcaacaagactgaagagaacagcaagaagaagatacacaagaagaaagaacagaatctgctacctgcaggaaatcttcagcaacgaaatggcaaaggtcgacgacagcttcttccacagactggaagaaagcttcctggtcgaagaagacaagaagcacgaaagacacccgatcttcggaaacatcgtcgacgaagtcgcataccacgaaaagtacccgacaatctaccacctgagaaagaagctggtcgacagcacagacaaggcagacctgagactgatctacctggcactggcacacatgatcaagttcagaggacacttcctgatcgaaggagacctgaacccggacaacagcgacgtcgacaagctgttcatccagctggtccagacatacaaccagctgttcgaagaaaacccgatcaacgcaagcggagtcgacgcaaaggcaatcctgagcgcaagactgagcaagagcagaagactggaaaacctgatcgcacagctgccgggagaaaagaagaacggactgttcggaaacctgatcgcactgagcctgggactgacaccgaacttcaagagcaacttcgacctggcagaagacgcaaagctgcagctgagcaaggacacatacgacgacgacctggacaacctgctggcacagatcggagaccagtacgcagacctgttcctggcagcaaagaacctgagcgacgcaatcctgctgagcgacatcctgagagtcaacacagaaatcacaaaggcaccgctgagcgcaagcatgatcaagagatacgacgaacaccaccaggacctgacactgctgaaggcactggtcagacagcagctgccggaaaagtacaaggaaatcttcttcgaccagagcaagaacggatacgcaggatacatcgacggaggagcaagccaggaagaattctacaagttcatcaagccgatcctggaaaagatggacggaacagaagaactgctggtcaagctgaacagagaagacctgctgagaaagcagagaacattcgacaacggaagcatcccgcaccagatccacctgggagaactgcacgcaatcctgagaagacaggaagacttctacccgttcctgaaggacaacagagaaaagatcgaaaagatcctgacattcagaatcccgtactacgtcggaccgctggcaagaggaaacagcagattcgcatggatgacaagaaagagcgaagaaacaatcacaccgtggaacttcgaagaagtcgtcgacaagggagcaagcgcacagagcttcatcgaaagaatgacaaacttcgacaagaacctgccgaacgaaaaggtcctgccgaagcacagcctgctgtacgaatacttcacagtctacaacgaactgacaaaggtcaagtacgtcacagaaggaatgagaaagccggcattcctgagcggagaacagaagaaggcaatcgtcgacctgctgttcaagacaaacagaaaggtcacagtcaagcagctgaaggaagactacttcaagaagatcgaatgcttcgacagcgtcgaaatcagcggagtcgaagacagattcaacgcaagcctgggaacataccacgacctgctgaagatcatcaaggacaaggacttcctggacaacgaagaaaacgaagacatcctggaagacatcgtcctgacactgacactgttcgaagacagagaaatgatcgaagaaagactgaagacatacgcacacctgttcgacgacaaggtcatgaagcagctgaagagaagaagatacacaggatggggaagactgagcagaaagctgatcaacggaatcagagacaagcagagcggaaagacaatcctggacttcctgaagagcgacggattcgcaaacagaaacttcatgcagctgatccacgacgacagcctgacattcaaggaagacatccagaaggcacaggtcagcggacagggagacagcctgcacgaacacatcgcaaacctggcaggaagcccggcaatcaagaagggaatcctgcagacagtcaaggtcgtcgacgaactggtcaaggtcatgggaagacacaagccggaaaacatcgtcatcgaaatggcaagagaaaaccagacaacacagaagggacagaagaacagcagagaaagaatgaagagaatcgaagaaggaatcaaggaactgggaagccagatcctgaaggaacacccggtcgaaaacacacagctgcagaacgaaaagctgtacctgtactacctgcagaacggaagagacatgtacgtcgaccaggaactggacatcaacagactgagcgactacgacgtcgaccacatcgtcccgcagagcttcctgaaggacgacagcatcgacaacaaggtcctgacaagaagcgacaagaacagaggaaagagcgacaacgtcccgagcgaagaagtcgtcaagaagatgaagaactactggagacagctgctgaacgcaaagctgatcacacagagaaagttcgacaacctgacaaaggcagagagaggaggactgagcgaactggacaaggcaggattcatcaagagacagctggtcgaaacaagacagatcacaaagcacgtcgcacagatcctggacagcagaatgaacacaaagtacgacgaaaacgacaagctgatcagagaagtcaaggtcatcacactgaagagcaagctggtcagcgacttcagaaaggacttccagttctacaaggtcagagaaatcaacaactaccaccacgcacacgacgcatacctgaacgcagtcgtcggaacagcactgatcaagaagtacccgaagctggaaagcgaattcgtctacggagactacaaggtctacgacgtcagaaagatgatcgcaaagagcgaacaggaaatcggaaaggcaacagcaaagtacttcttctacagcaacatcatgaacttcttcaagacagaaatcacactggcaaacggagaaatcagaaagagaccgctgatcgaaacaaacggagaaacaggagaaatcgtctgggacaagggaagagacttcgcaacagtcagaaaggtcctgagcatgccgcaggtcaacatcgtcaagaagacagaagtccagacaggaggattcagcaaggaaagcatcctgccgaagagaaacagcgacaagctgatcgcaagaaagaaggactgggacccgaagaagtacggaggattcgacagcccgacagtcgcatacagcgtcctggtcgtcgcaaaggtcgaaaagggaaagagcaagaagctgaagagcgtcaaggaactgctgggaatcacaatcatggaaagaagcagcttcgaaaagaacccgatcgacttcctggaagcaaagggatacaaggaagtcaagaaggacctgatcatcaagctgccgaagtacagcctgttcgaactggaaaacggaagaaagagaatgctggcaagcgcaggagaactgcagaagggaaacgaactggcactgccgagcaagtacgtcaacttcctgtacctggcaagccactacgaaaagctgaagggaagcccggaagacaacgaacagaagcagctgttcgtcgaacagcacaagcactacctggacgaaatcatcgaacagatcagcgaattcagcaagagagtcatcctggcagacgcaaacctggacaaggtcctgagcgcatacaacaagcacagagacaagccgatcagagaacaggcagaaaacatcatccacctgttcacactgacaaacctgggagcaccggcagcattcaagtacttcgacacaacaatcgacagaaagagatacacaagcacaaaggaagtcctggacgcaacactgatccaccagagcatcacaggactgtacgaaacaagaatcgacctgagccagctgggaggagacggaggaggaagcccgaagaagaagagaaaggtctag

SEQ ID NO: 322 - RNA, 4140 nt, synthetic construct
  misc_feature 1..4140: note = Synthetic
  source 1..4140: mol_type = other RNA
atggacaagaagtactccatcggcctggacatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtga

SEQ ID NO: 323 - RNA, 4197 nt, synthetic construct
  misc_feature 1..4197: note = Synthetic
  source 1..4197: mol_type = other RNA
atggacaagaagtactccatcggcctggacatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtccgagtccgccacccccgagtccgtgtccggctggcggctgttcaagaagatctcctga

SEQ ID NO: 324-349 - skipped (26 entries)

SEQ ID NO: 350 - protein, 1105 aa, synthetic construct
  REGION 1..1105: note = Synthetic
MTGAAFKPNPINYILGLDIGIASVGWAMVEIDEEENPIRLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVANNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSSELQDEIGTAFSLFKTDEDITGRLKDRVQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPRSKKQRILLQKFDEDGFKECNLNDTRYVNRFLCQFVADHILLTGKGKRRVFASNGQITNLLRGFWGLRKVRAENDRHHALDAVVVACSTVAMQQKITRFVRYKEMNAFDGKTIDKETGKVLHQKTHFPQPWEFFAQEVMIRVFGKPDGKPEFEEADTPEKLRTLLAEKLSSRPEAVHEYVTPLFVSRAPNRKMSGAHKDTLRSAKRFVKHNEKISVKRVWLTEIKLADLENMVNYKNGREIELYEALKARLEAYGGNAKQAFDPKDNPFYKKGGQLVKAVRVEKTQESGVLLNKKNAYTIADNGDMVRVDVFCKVDKKGKNQYFIVPIYAWQVAENILPDIDCKGYRIDDSYTFCFSLHKYDLIAFQKDEKSKVEFAYYINCDSSNGRFYLAWHDKGSKEQQFRISTQNLVLIQKYQVNELGKEIRPCRLKKRPPVRSGKRTADGSEFESPKKKRKVE

SEQ ID NO: 351 - protein, 107 aa, synthetic construct
  REGION 1..107: note = Synthetic
MTNLSDIIEKETGKQLVIQESILMLPEEVEEVIGNKPESDILVHTAYDESTDENVMLLTSDAPEYKPWALVIQDSNGENKIKMLSGGSKRTADGSEFESPKKKRKVE

SEQ ID NO: 352 - protein, 1110 aa, synthetic construct
  REGION 1..1110: note = Synthetic
MVPKKKRKVAAFKPNPINYILGLDIGIASVGWAMVEIDEEENPIRLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVANNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSSELQDEIGTAFSLFKTDEDITGRLKDRVQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPRSKKQRILLQKFDEDGFKECNLNDTRYVNRFLCQFVADHILLTGKGKRRVFASNGQITNLLRGFWGLRKVRAENDRHHALDAVVVACSTVAMQQKITRFVRYKEMNAFDGKTIDKETGKVLHQKTHFPQPWEFFAQEVMIRVFGKPDGKPEFEEADTPEKLRTLLAEKLSSRPEAVHEYVTPLFVSRAPNRKMSGAHKDTLRSAKRFVKHNEKISVKRVWLTEIKLADLENMVNYKNGREIELYEALKARLEAYGGNAKQAFDPKDNPFYKKGGQLVKAVRVEKTQESGVLLNKKNAYTIADNGDMVRVDVFCKVDKKGKNQYFIVPIYAWQVAENILPDIDCKGYRIDDSYTFCFSLHKYDLIAFQKDEKSKVEFAYYINCDSSNGRFYLAWHDKGSKEQQFRISTQNLVLIQKYQVNELGKEIRPCRLKKRPPVRYPYDVPDYAAAPAAKKKKLD

SEQ ID NO: 353 - protein, 1082 aa, synthetic construct; identical to SEQ ID NO: 310
  REGION 1..1082: note = Synthetic

SEQ ID NO: 354 - protein, 1121 aa, synthetic construct
  REGION 1..1121: note = Synthetic
MDGSGGGSPKKKRKVGGSGGGAAFKPNPINYILGLDIGIASVGWAMVEIDEEENPIRLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVANNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSSELQDEIGTAFSLFKTDEDITGRLKDRVQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPRSKKQRILLQKFDEDGFKECNLNDTRYVNRFLCQFVADHILLTGKGKRRVFASNGQITNLLRGFWGLRKVRAENDRHHALDAVVVACSTVAMQQKITRFVRYKEMNAFDGKTIDKETGKVLHQKTHFPQPWEFFAQEVMIRVFGKPDGKPEFEEADTPEKLRTLLAEKLSSRPEAVHEYVTPLFVSRAPNRKMSGAHKDTLRSAKRFVKHNEKISVKRVWLTEIKLADLENMVNYKNGREIELYEALKARLEAYGGNAKQAFDPKDNPFYKKGGQLVKAVRVEKTQESGVLLNKKNAYTIADNGDMVRVDVFCKVDKKGKNQYFIVPIYAWQVAENILPDIDCKGYRIDDSYTFCFSLHKYDLIAFQKDEKSKVEFAYYINCDSSNGRFYLAWHDKGSKEQQFRISTQNLVLIQKYQVNELGKEIRPCRLKKRPPVRSESATPESVSGWRLFKKIS

SEQ ID NO: 355 - protein, 1124 aa, synthetic construct
  REGION 1..1124: note = Synthetic
MDGSGGGSPKKKRKVGGSGGGAAFKPNPINYILGLDIGIASVGWAMVEIDEEENPIRLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVANNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSSELQDEIGTAFSLFKTDEDITGRLKDRVQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPRSKKQRILLQKFDEDGFKECNLNDTRYVNRFLCQFVADHILLTGKGKRRVFASNGQITNLLRGFWGLRKVRAENDRHHALDAVVVACSTVAMQQKITRFVRYKEMNAFDGKTIDKETGKVLHQKTHFPQPWEFFAQEVMIRVFGKPDGKPEFEEADTPEKLRTLLAEKLSSRPEAVHEYVTPLFVSRAPNRKMSGAHKDTLRSAKRFVKHNEKISVKRVWLTEIKLADLENMVNYKNGREIELYEALKARLEAYGGNAKQAFDPKDNPFYKKGGQLVKAVRVEKTQESGVLLNKKNAYTIADNGDMVRVDVFCKVDKKGKNQYFIVPIYAWQVAENILPDIDCKGYRIDDSYTFCFSLHKYDLIAFQKDEKSKVEFAYYINCDSSNGRFYLAWHDKGSKEQQFRISTQNLVLIQKYQVNELGKEIRPCRLKKRPPVRSGKRTADGSGGGSPAAKKKKLD

SEQ ID NO: 356 - protein, 1120 aa, synthetic construct
  REGION 1..1120: note = Synthetic
MDGSGGGSPKKKRKVEDKRPAATKKAGQAKKKKGGSGGGAAFKPNPINYILGLDIGIASVGWAMVEIDEEENPIRLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVANNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSSELQDEIGTAFSLFKTDEDITGRLKDRVQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPRSKKQRILLQKFDEDGFKECNLNDTRYVNRFLCQFVADHILLTGKGKRRVFASNGQITNLLRGFWGLRKVRAENDRHHALDAVVVACSTVAMQQKITRFVRYKEMNAFDGKTIDKETGKVLHQKTHFPQPWEFFAQEVMIRVFGKPDGKPEFEEADTPEKLRTLLAEKLSSRPEAVHEYVTPLFVSRAPNRKMSGAHKDTLRSAKRFVKHNEKISVKRVWLTEIKLADLENMVNYKNGREIELYEALKARLEAYGGNAKQAFDPKDNPFYKKGGQLVKAVRVEKTQESGVLLNKKNAYTIADNGDMVRVDVFCKVDKKGKNQYFIVPIYAWQVAENILPDIDCKGYRIDDSYTFCFSLHKYDLIAFQKDEKSKVEFAYYINCDSSNGRFYLAWHDKGSKEQQFRISTQNLVLIQKYQVNELGKEIRPCRLKKRPPVR

SEQ ID NO: 357 - protein, 1139 aa, synthetic construct
  REGION 1..1139: note = Synthetic
MDGSGGGSPKKKRKVEDKRPAATKKAGQAKKKKGGSGGGAAFKPNPINYILGLDIGIASVGWAMVEIDEEENPIRLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVANNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSSELQDEIGTAFSLFKTDEDITGRLKDRVQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPRSKKQRILLQKFDEDGFKECNLNDTRYVNRFLCQFVADHILLTGKGKRRVFASNGQITNLLRGFWGLRKVRAENDRHHALDAVVVACSTVAMQQKITRFVRYKEMNAFDGKTIDKETGKVLHQKTHFPQPWEFFAQEVMIRVFGKPDGKPEFEEADTPEKLRTLLAEKLSSRPEAVHEYVTPLFVSRAPNRKMSGAHKDTLRSAKRFVKHNEKISVKRVWLTEIKLADLENMVNYKNGREIELYEALKARLEAYGGNAKQAFDPKDNPFYKKGGQLVKAVRVEKTQESGVLLNKKNAYTIADNGDMVRVDVFCKVDKKGKNQYFIVPIYAWQVAENILPDIDCKGYRIDDSYTFCFSLHKYDLIAFQKDEKSKVEFAYYINCDSSNGRFYLAWHDKGSKEQQFRISTQNLVLIQKYQVNELGKEIRPCRLKKRPPVRSESATPESVSGWRLFKKIS

SEQ ID NO: 358 - protein, 1113 aa, synthetic construct
  REGION 1..1113: note = Synthetic
MDGSGGGSEDKRPAATKKAGQAKKKKGGSGGGAAFKPNPINYILGLDIGIASVGWAMVEIDEEENPIRLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVANNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSSELQDEIGTAFSLFKTDEDITGRLKDRVQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPRSKKQRILLQKFDEDGFKECNLNDTRYVNRFLCQFVADHILLTGKGKRRVFASNGQITNLLRGFWGLRKVRAENDRHHALDAVVVACSTVAMQQKITRFVRYKEMNAFDGKTIDKETGKVLHQKTHFPQPWEFFAQEVMIRVFGKPDGKPEFEEADTPEKLRTLLAEKLSSRPEAVHEYVTPLFVSRAPNRKMSGAHKDTLRSAKRFVKHNEKISVKRVWLTEIKLADLENMVNYKNGREIELYEALKARLEAYGGNAKQAFDPKDNPFYKKGGQLVKAVRVEKTQESGVLLNKKNAYTIADNGDMVRVDVFCKVDKKGKNQYFIVPIYAWQVAENILPDIDCKGYRIDDSYTFCFSLHKYDLIAFQKDEKSKVEFAYYINCDSSNGRFYLAWHDKGSKEQQFRISTQNLVLIQKYQVNELGKEIRPCRLKKRPPVR

SEQ ID NO: 359 - protein, 1334 aa, synthetic construct
  REGION 1..1334: note = Synthetic
MDGSGGGSPKKKRKVEDKRPAATKKAGQAKKKKGGSGGGEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSGSETPGTSESATPESAAFKPNPINYILGLAIGIASVGWAMVEIDEEENPIRLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVANNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSSELQDEIGTAFSLFKTDEDITGRLKDRVQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPRSKKQRILLQKFDEDGFKECNLNDTRYVNRFLCQFVADHILLTGKGKRRVFASNGQITNLLRGFWGLRKVRAENDRHHALDAVVVACSTVAMQQKITRFVRYKEMNAFDGKTIDKETGKVLHQKTHFPQPWEFFAQEVMIRVFGKPDGKPEFEEADTPEKLRTLLAEKLSSRPEAVHEYVTPLFVSRAPNRKMSGAHKDTLRSAKRFVKHNEKISVKRVWLTEIKLADLENMVNYKNGREIELYEALKARLEAYGGNAKQAFDPKDNPFYKKGGQLVKAVRVEKTQESGVLLNKKNAYTIADNGDMVRVDVFCKVDKKGKNQYFIVPIYAWQVAENILPDIDCKGYRIDDSYTFCFSLHKYDLIAFQKDEKSKVEFAYYINCDSSNGRFYLAWHDKGSKEQQFRISTQNLVLIQKYQVNELGKEIRPCRLKKRPPVR

SEQ ID NO: 360 - protein, 1593 aa, synthetic construct
  REGION 1..1593: note = Synthetic
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSGSETPGTSESATPESDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGSPKKKRKV

SEQ ID NO: 361 - RNA, 3837 nt, synthetic construct
  misc_feature 1..3837: note = Synthetic
  source 1..3837: mol_type = other RNA
gggaagctcagaataaacgctcaactttggccggatctgccaccatgaccggtgccgccttcaagcccaaccccatcaactacatcctgggcctggacatcggcatcgcctccgtgggctgggccatggtggagatcgacgaggaggagaaccccatccggctgatcgacctgggcgtgcgggtgttcgagcgggccgaggtgcccaagaccggcgactccctggccatggcccggcggctggcccggtccgtgcggcggctgacccggcggcgggcccaccggctgctgcgggcccggcggctgctgaagcgggagggcgtgctgcaggccgccgacttcgacgagaacggcctgatcaagtccctgcccaacaccccctggcagctgcgggccgccgccctggaccggaagctgacccccctggagtggtccgccgtgctgctgcacctgatcaagcaccggggctacctgtcccagcggaagaacgagggcgagaccgccgacaaggagctgggcgccctgctgaagggcgtggccaacaacgcccacgccctgcagaccggcgacttccggacccccgccgagctggccctgaacaagttcgagaaggagtccggccacatccggaaccagcggggcgactactcccacaccttctcccggaaggacctgcaggccgagctgatcctgctgttcgagaagcagaaggagttcggcaacccccacgtgtccggcggcctgaaggagggcatcgagaccctgctgatgacccagcggcccgccctgtccggcgacgccgtgcagaagatgctgggccactgcaccttcgagcccgccgagcccaaggccgccaagaacacctacaccgccgagcggttcatctggctgaccaagctgaacaacctgcggatcctggagcagggctccgagcggcccctgaccgacaccgagcgggccaccctgatggacgagccctaccggaagtccaagctgacctacgcccaggcccggaagctgctgggcctggaggacaccgccttcttcaagggcctgcggtacggcaaggacaacgccgaggcctccaccctgatggagatgaaggcctaccacgccatctcccgggccctggagaaggagggcctgaaggacaagaagtcccccctgaacctgtcctccgagctgcaggacgagatcggcaccgccttctccctgttcaagaccgacgaggacatcaccggccggctgaaggaccgggtgcagcccgagatcctggaggccctgctgaagcacatctccttcgacaagttcgtgcagatctccctgaaggccctgcggcggatcgtgcccctgatggagcagggcaagcggtacgacgaggcctgcgccgagatctacggcgaccactacggcaagaagaacaccgaggagaagatctacctgccccccatccccgccgacgagatccggaaccccgtggtgctgcgggccctgtcccaggcccggaaggtgatcaacggcgtggtgcggcggtacggctcccccgcccggatccacatcgagaccgcccgggaggtgggcaagtccttcaaggaccggaaggagatcgagaagcggcaggaggagaaccggaaggaccgggagaaggccgccgccaagttccgggagtacttccccaacttcgtgggcgagcccaagtccaaggacatcctgaagctgcggctgtacgagcagcagcacggcaagtgcctgtactccggcaaggagatcaacctggtgcggctgaacgagaagggctacgtggagatcgaccacgccctgcccttctcccggacctgggacgactccttcaacaacaaggtgctggtgctgggctccgagaaccagaacaagggcaaccagaccccctacgagtacttcaacggcaaggacaactcccgggagtggcaggagttcaaggcccgggtggagacctcccggttcccccggtccaagaagcagcggatcctgctgcagaagttcgacgaggacggcttcaaggagtgcaacctgaacgacacccggtacgtgaaccgcttcctgtgccagttcgtggccgaccacatcctgctgaccggcaagggcaagcggcgggtgttcgcctccaacggccagatcaccaacctgctgcggggcttctggggcctgcggaaggtgcgggccgagaacgaccggcaccacgccctggacgccgtggtggtggcctgctccaccgtggccatgcagcagaagatcacccggttcgtgcggtacaaggagatgaacgccttcgacggcaagaccatcgacaaggagaccggcaaggtgctgcaccagaagacccacttcccccagccctgggagttcttcgcccaggaggtgatgatccgggtgttcggcaagcccgacggcaagcccgagttcgaggaggccgacacccccgagaagctgcggaccctgctggccgagaagctgtcctcccggcccgaggccgtgcacgagtacgtgacccccctgttcgtgtcccgggcccccaaccggaagatgtccggcgcccacaaggacaccctgcggtccgccaagcggttcgtgaagcacaacgagaagatctccgtgaagcgggtgtggctgaccgagatcaagctggccgacctggagaacatggtgaactacaagaacggccgggagatcgagctgtacgaggccctgaaggcccggctggaggcctacggcggcaacgccaagcaggccttcgaccccaaggacaaccccttctacaagaagggcggccagctggtgaaggccgtgcgggtggagaagacccaggagtccggcgtgctgctgaacaagaagaacgcctacaccatcgccgacaacggcgacatggtgcgggtggacgtgttctgcaaggtggacaagaagggcaagaaccagtacttcatcgtgcccatctacgcctggcaggtggccgagaacatcctgcccgacatcgactgcaagggctaccggatcgacgactcctacaccttctgcttctccctgcacaagtacgacctgatcgccttccagaaggacgagaagtccaaggtggagttcgcctactacatcaactgcgactcctccaacggccggttctacctggcctggcacgacaagggctccaaggagcagcagttccggatctccacccagaacctggtgctgatccagaagtaccaggtgaacgagctgggcaaggagatccggccctgccggctgaagaagcggccccccgtgcggtccggaaagcggaccgccgacggctccgagttcgagtcccccaagaagaagcggaaggtggagtagctagcaccagcctcaagaacacccgaatggagtctctaagctacataataccaacttacactttacaaaatgttgtcccccaaaatgtagccattcgtatctgctcctaataaaaagaaagtttcttcacattctctcgagaaaaaaaaaaaatggaaaaaaaaaaaacggaaaaaaaaaaaaggtaaaaaaaaaaaatataaaaaaaaaaaacataaaaaaaaaaaacgaaaaaaaaaaaacgtaaaaaaaaaaaactcaaaaaaaaaaaagataaaaaaaaaaaacctaaaaaaaaaaaatgtaaaaaaaaaaaagggaaaaaaaaaaaacgcaaaaaaaaaaaacacaaaaaaaaaaaatgcaaaaaaaaaaaatcgaaaaaaaaaaaatctaaaaaaaaaaaacgaaaaaaaaaaaacccaaaaaaaaaaaagacaaaaaaaaaaaatagaaaaaaaaaaaagttaaaaaa

SEQ ID NO: 362 - RNA, 887 nt, synthetic construct
  misc_feature 1..887: note = Synthetic
  source 1..887: mol_type = other RNA
gggaagctcagaataaacgctcaactttggccggatctgccaccatgaccaacctgtccgacatcatcgagaaggagaccggcaagcagctggtgatccaggagtccatcctgatgctgcccgaggaggtggaggaggtgatcggcaacaagcccgagtccgacatcctggtgcacaccgcctacgacgagtccaccgacgagaacgtgatgctgctgacctccgacgcccccgagtacaagccctgggccctggtgatccaggactccaacggcgagaacaagatcaagatgctgtccggcggctccaagcggaccgccgacggctccgagttcgagtcccccaagaagaagcggaaggtggagtgatagctagcaccagcctcaagaacacccgaatggagtctctaagctacataataccaacttacactttacaaaatgttgtcccccaaaatgtagccattcgtatctgctcctaataaaaagaaagtttcttcacattctctcgagaaaaaaaaaaaatggaaaaaaaaaaaacggaaaaaaaaaaaaggtaaaaaaaaaaaatataaaaaaaaaaaacataaaaaaaaaaaacgaaaaaaaaaaaacgtaaaaaaaaaaaactcaaaaaaaaaaaagataaaaaaaaaaaacctaaaaaaaaaaaatgtaaaaaaaaaaaagggaaaaaaaaaaaacgcaaaaaaaaaaaacacaaaaaaaaaaaatgcaaaaaaaaaaaatcgaaaaaaaaaaaatctaaaaaaaaaaaacgaaaaaaaaaaaacccaaaaaaaaaaaagacaaaaaaaaaaaatagaaaaaaaaaaaagttaaaaaaaaaaaactgaaaaaaaaaaaatttaaaaaaaaaaaatctag

SEQ ID NO: 363 - DNA, 3889 bp, synthetic construct
  misc_feature 1..3889: note = Synthetic
  source 1..3889: mol_type = other DNA
gggaagctcagaataaacgctcaactttggccggatctgccaccatggtgcccaagaagaagcggaaggtggccgccttcaagcccaaccccatcaactacatcctgggcctggacatcggcatcgcctccgtgggctgggccatggtggagatcgacgaggaggagaaccccatccggctgatcgacctgggcgtgcgggtgttcgagcgggccgaggtgcccaagaccggcgactccctggccatggcccggcggctggcccggtccgtgcggcggctgacccggcggcgggcccaccggctgctgcgggcccggcggctgctgaagcgggagggcgtgctgcaggccgccgacttcgacgagaacggcctgatcaagtccctgcccaacaccccctggcagctgcgggccgccgccctggaccggaagctgacccccctggagtggtccgccgtgctgctgcacctgatcaagcaccggggctacctgtcccagcggaagaacgagggcgagaccgccgacaaggagctgggcgccctgctgaagggcgtggccaacaacgcccacgccctgcagaccggcgacttccggacccccgccgagctggccctgaacaagttcgagaaggagtccggccacatccggaaccagcggggcgactactcccacaccttctcccggaaggacctgcaggccgagctgatcctgctgttcgagaagcagaaggagttcggcaacccccacgtgtccggcggcctgaaggagggcatcgagaccctgctgatgacccagcggcccgccctgtccggcgacgccgtgcagaagatgctgggccactgcaccttcgagcccgccgagcccaaggccgccaagaacacctacaccgccgagcggttcatctggctgaccaagctgaacaacctgcggatcctggagcagggctccgagcggcccctgaccgacaccgagcgggccaccctgatggacgagccctaccggaagtccaagctgacctacgcccaggcccggaagctgctgggcctggaggacaccgccttcttcaagggcctgcggtacggcaaggacaacgccgaggcctccaccctgatggagatgaaggcctaccacgccatctcccgggccctggagaaggagggcctgaaggacaagaagtcccccctgaacctgtcctccgagctgcaggacgagatcggcaccgccttctccctgttcaagaccgacgaggacatcaccggccggctgaaggaccgggtgcagcccgagatcctggaggccctgctgaagcacatctccttcgacaagttcgtgcagatctccctgaaggccctgcggcggatcgtgcccctgatggagcagggcaagcggtacgacgaggcctgcgccgagatctacggcgaccactacggcaagaagaacaccgaggagaagatctacctgccccccatccccgccgacgagatccggaaccccgtggtgctgcgggccctgtcccaggcccggaaggtgatcaacggcgtggtgcggcggtacggctcccccgcccggatccacatcgagaccgcccgggaggtgggcaagtccttcaaggaccggaaggagatcgagaagcggcaggaggagaaccggaaggaccgggagaaggccgccgccaagttccgggagtacttccccaacttcgtgggcgagcccaagtccaaggacatcctgaagctgcggctgtacgagcagcagcacggcaagtgcctgtactccggcaaggagatcaacctggtgcggctgaacgagaagggctacgtggagatcgaccacgccctgcccttctcccggacctgggacgactccttcaacaacaaggtgctggtgctgggctccgagaaccagaacaagggcaaccagaccccctacgagtacttcaacggcaaggacaactcccgggagtggcaggagttcaaggcccgggtggagacctcccggttcccccggtccaagaagcagcggatcctgctgcagaagttcgacgaggacggcttcaaggagtgcaacctgaacgacacccggtacgtgaaccggttcctgtgccagttcgtggccgaccacatcctgctgaccggcaagggcaagcggcgggtgttcgcctccaacggccagatcaccaacctgctgcggggcttctggggcctgcggaaggtgcgggccgagaacgaccggcaccacgccctggacgccgtggtggtggcctgctccaccgtggccatgcagcagaagatcacccggttcgtgcggtacaaggagatgaacgccttcgacggcaagaccatcgacaaggagaccggcaaggtgctgcaccagaagacccacttcccccagccctgggagttcttcgcccaggaggtgatgatccgggtgttcggcaagcccgacggcaagcccgagttcgaggaggccgacacccccgagaagctgcggaccctgctggccgagaagctgtcctcccggcccgaggccgtgcacgagtacgtgacccccctgttcgtgtcccgggcccccaaccggaagatgtccggcgcccacaaggacaccctgcggtccgccaagcggttcgtgaagcacaacgagaagatctccgtgaagcgggtgtggctgaccgagatcaagctggccgacctggagaacatggtgaactacaagaacggccgggagatcgagctgtacgaggccctgaaggcccggctggaggcctacggcggcaacgccaagcaggccttcgaccccaaggacaaccccttctacaagaagggcggccagctggtgaaggccgtgcgggtggagaagacccaggagtccggcgtgctgctgaacaagaagaacgcctacaccatcgccgacaacggcgacatggtgcgggtggacgtgttctgcaaggtggacaagaagggcaagaaccagtacttcatcgtgcccatctacgcctggcaggtggccgagaacatcctgcccgacatcgactgcaagggctaccggatcgacgactcctacaccttctgcttctccctgcacaagtacgacctgatcgccttccagaaggacgagaagtccaaggtggagttcgcctactacatcaactgcgactcctccaacggccggttctacctggcctggcacgacaagggctccaaggagcagcagttccggatctccacccagaacctggtgctgatccagaagtaccaggtgaacgagctgggcaaggagatccggccctgccggctgaagaagcggccccccgtgcggtacccctacgacgtgcccgactacgccgccgcccccgccgccaagaagaagaagctggactagctagcaccagcctcaagaacacccgaatggagtctctaagctacataataccaacttacactttacaaaatgttgtcccccaaaatgtagccattcgtatctgctcctaataaaaagaaagtttcttcacattctctcgagaaaaaaaaaaaatggaaaaaaaaaaaacggaaaaaaaaaaaaggtaaaaaaaaaaaatataaaaaaaaaaaacataaaaaaaaaaaacgaaaaaaaaaaaacgtaaaaaaaaaaaactcaaaaaaaaaaaagataaaaaaaaaaaacctaaaaaaaaaaaatgtaaaaaaaaaaaagggaaaaaaaaaaaacgcaaaaaaaaaaaacacaaaaaaaaaaaatgcaaaaaaaaaaaatcgaaaaaaaaaaaatctaaaaaaaaaaaacgaaaaaaaaaaaacccaaaaaaaaaaaagacaaaaaaaaaaaatagaaaaaaaaaaaagttaaaaaaaaaaaactgaaaaaaaaaaaatttaaaaaaaaaaaat

SEQ ID NO: 364 - RNA, 4067 nt, synthetic construct
  misc_feature 1..4067: note = Synthetic
  source 1..4067: mol_type = other RNA
gggaagctcagaataaacgctcaactttggccggatctgccaccatggtgcccaagaagaagcggaaggtggaggacaagcggcccgccgccaccaagaaggccggccaggccaagaagaagaagatggccgccttcaagcccaaccccatcaactacatcctgggcctggacatcggcatcgcctccgtgggctgggccatggtggagatcgacgaggaggagaaccccatccggctgatcgacctgggcgtgcgggtgttcgagcgggccgaggtgcccaagaccggcgactccctggccatggcccggcggctggcccggtccgtgcggcggctgacccggcggcgggcccaccggctgctgcgggcccggcggctgctgaagcgggagggcgtgctgcaggccgccgacttcgacgagaacggcctgatcaagtccctgcccaacaccccctggcagctgcgggccgccgccctggaccggaagctgacccccctggagtggtccgccgtgctgctgcacctgatcaagcaccggggctacctgtcccagcggaagaacgagggcgagaccgccgacaaggagctgggcgccctgctgaagggcgtggccaacaacgcccacgccctgcagaccggcgacttccggacccccgccgagctggccctgaacaagttcgagaaggagtccggccacatccggaaccagcggggcgactactcccacaccttctcccggaaggacctgcaggccgagctgatcctgctgttcgagaagcagaaggagttcggcaacccccacgtgtccggcggcctgaaggagggcatcgagaccctgctgatgacccagcggcccgccctgtccggcgacgccgtgcagaagatgctgggccactgcaccttcgagcccgccgagcccaaggccgccaagaacacctacaccgccgagcggttcatctggctgaccaagctgaacaacctgcggatcctggagcagggctccgagcggcccctgaccgacaccgagcgggccaccctgatggacgagccctaccggaagtccaagctgacctacgcccaggcccggaagctgctgggcctggaggacaccgccttcttcaagggcctgcggtacggcaaggacaacgccgaggcctccaccctgatggagatgaaggcctaccacgccatctcccgggccctggagaaggagggcctgaaggacaagaagtcccccctgaacctgtcctccgagctgcaggacgagatcggcaccgccttctccctgttcaagaccgacgaggacatcaccggccggctgaaggaccgggtgcagcccgagatcctggaggccctgctgaagcacatctccttcgacaagttcgtgcagatctccctgaaggccctgcggcggatcgtgcccctgatggagcagggcaagcggtacgacgaggcctgcgccgagatctacggcgaccactacggcaagaagaacaccgaggagaagatctacctgccccccatccccgccgacgagatccggaaccccgtggtgctgcgggccctgtcccaggcccggaaggtgatcaacggcgtggtgcggcggtacggctcccccgcccggatccacatcgagaccgcccgggaggtgggcaagtccttcaaggaccggaaggagatcgagaagcggcaggaggagaaccggaaggaccgggagaaggccgccgccaagttccgggagtacttccccaacttcgtgggcgagcccaagtccaaggacatcctgaagctgcggctgtacgagcagcagcacggcaagtgcctgtactccggcaaggagatcaacctggtgcggctgaacgagaagggctacgtggagatcgaccacgccctgcccttctcccggacctgggacgactccttcaacaacaaggtgctggtgctgggctccgagaaccagaacaagggcaaccagaccccctacgagtacttcaacggcaaggacaactcccgggagtggcaggagttcaaggcccgggtggagacctcccggttcccccggtccaagaagcagcggatcctgctgcagaagttcgacgaggacggcttcaaggagtgcaacctgaacgacacccggtacgtgaaccggttcctgtgccagttcgtggccgaccacatcctgctgaccggcaagggcaagcggcgggtgttcgcctccaacggccagatcaccaacctgctgcggggcttctggggcctgcggaaggtgcgggccgagaacgaccggcaccacgccctggacgccgtggtggtggcctgctccaccgtggccatgcagcagaagatcacccggttcgtgcggtacaaggagatgaacgccttcgacggcaagaccatcgacaaggagaccggcaaggtgctgcaccagaagacccacttcccccagccctgggagttcttcgcccaggaggtgatgatccgggtgttcggcaagcccgacggcaagcccgagttcgaggaggccgacacccccgagaagctgcggaccctgctggccgagaagctgtcctcccggcccgaggccgtgcacgagtacgtgacccccctgttcgtgtcccgggcccccaaccggaagatgtccggcgcccacaaggacaccctgcggtccgccaagcggttcgtgaagcacaacgagaagatctccgtgaagcgggtgtggctgaccgagatcaagctggccgacctggagaacatggtgaactacaagaacggccgggagatcgagctgtacgaggccctgaaggcccggctggaggcctacggcggcaacgccaagcaggccttcgaccccaaggacaaccccttctacaagaagggcggccagctggtgaaggccgtgcgggtggagaagacccaggagtccggcgtgctgctgaacaagaagaacgcctacaccatcgccgacaacggcgacatggtgcgggtggacgtgttctgcaaggtggacaagaagggcaagaaccagtacttcatcgtgcccatctacgcctggcaggtggccgagaacatcctgcccgacatcgactgcaagggctaccggatcgacgactcctacaccttctgcttctccctgcacaagtacgacctgatcgccttccagaaggacgagaagtccaaggtggagttcgcctactacatcaactgcgactcctccaacggccggttctacctggcctggcacgacaagggctccaaggagcagcagttccggatctccacccagaacctggtgctgatccagaagtaccaggtgaacgagctgggcaaggagatccggccctgccggctgaagaagcggccccccgtgcgggaggacaagcggcccgccgccaccaagaaggccggccaggccaagaagaagaagtacccctacgacgtgcccgactacgccggctacccctacgacgtgcccgactacgccggctcctacccctacgacgtgcccgactacgccgccgcccccgccgccaagaagaagaagctggactagctagcaccagcctcaagaacacccgaatggagtctctaagctacataataccaacttacactttacaaaatgttgtcccccaaaatgtagccattcgtatctgctcctaataaaaagaaagtttcttcacattctctcgagaaaaaaaaaaaatggaaaaaaaaaaaacggaaaaaaaaaaaaggtaaaaaaaaaaaatataaaaaaaaaaaacataaaaaaaaaaaacgaaaaaaaaaaaacgtaaaaaaaaaaaactcaaaaaaaaaaaagataaaaaaaaaaaacctaaaaaaaaaaaatgtaaaaaaaaaaaagggaaaaaaaaaaaacgcaaaaaaaaaaaacacaaaaaaaaaaaatgcaaaaaaaaaaaatcgaaaaaaaaaaaatctaaaaaaaaaaaacgaaaaaaaaaaaacccaaaaaaaaaaaagacaaaaaaaaaaaatagaaaaaaaaaaaagttaaaaaaaaaaaactgaaaaaaaaaaaatttaaaaaaaaaaaatctag

SEQ ID NO: 365 - RNA, 3926 nt, synthetic construct
  misc_feature 1..3926: note = Synthetic
  source 1..3926: mol_type = other RNA
gggaagctcagaataaacgctcaactttggccggatctgccaccatggacggctccggcggcggctcccccaagaagaagcggaaggtgggcggctccggcggcggcgccgccttcaagcccaaccccatcaactacatcctgggcctggacatcggcatcgcctccgtgggctgggccatggtggagatcgacgaggaggagaaccccatccggctgatcgacctgggcgtgcgggtgttcgagcgggccgaggtgcccaagaccggcgactccctggccatggcccggcggctggcccggtccgtgcggcggctgacccggcggcgggcccaccggctgctgcgggcccggcggctgctgaagcgggagggcgtgctgcaggccgccgacttcgacgagaacggcctgatcaagtccctgcccaacaccccctggcagctgcgggccgccgccctggaccggaagctgacccccctggagtggtccgccgtgctgctgcacctgatcaagcaccggggctacctgtcccagcggaagaacgagggcgagaccgccgacaaggagctgggcgccctgctgaagggcgtggccaacaacgcccacgccctgcagaccggcgacttccggacccccgccgagctggccctgaacaagttcgagaaggagtccggccacatccggaaccagcggggcgactactcccacaccttctcccggaaggacctgcaggccgagctgatcctgctgttcgagaagcagaaggagttcggcaacccccacgtgtccggcggcctgaaggagggcatcgagaccctgctgatgacccagcggcccgccctgtccggcgacgccgtgcagaagatgctgggccactgcaccttcgagcccgccgagcccaaggccgccaagaacacctacaccgccgagcggttcatctggctgaccaagctgaacaacctgcggatcctggagcagggctccgagcggcccctgaccgacaccgagcgggccaccctgatggacgagccctaccggaagtccaagctgacctacgcccaggcccggaagctgctgggcctggaggacaccgccttcttcaagggcctgcggtacggcaaggacaacgccgaggcctccaccctgatggagatgaaggcctaccacgccatctcccgggccctggagaaggagggcctgaaggacaagaagtcccccctgaacctgtcctccgagctgcaggacgagatcggcaccgccttctccctgttcaagaccgacgaggacatcaccggccggctgaaggaccgggtgcagcccgagatcctggaggccctgctgaagcacatctccttcgacaagttcgtgcagatctccctgaaggccctgcggcggatcgtgcccctgatggagcagggcaagcggtacgacgaggcctgcgccgagatctacggcgaccactacggcaagaagaacaccgaggagaagatctacctgccccccatccccgccgacgagatccggaaccccgtggtgctgcgggccctgtcccaggcccggaaggtgatcaacggcgtggtgcggcggtacggctcccccgcccggatccacatcgagaccgcccgggaggtgggcaagtccttcaaggaccggaaggagatcgagaagcggcaggaggagaaccggaaggaccgggagaaggccgccgccaagttccgggagtacttccccaacttcgtgggcgagcccaagtccaaggacatcctgaagctgcggctgtacgagcagcagcacggcaagtgcctgtactccggcaaggagatcaacctggtgcggctgaacgagaagggctacgtggagatcgaccacgccctgcccttctcccggacctgggacgactccttcaacaacaaggtgctggtgctgggctccgagaaccagaacaagggcaaccagaccccctacgagtacttcaacggcaaggacaactcccgggagtggcaggagttcaaggcccgggtggagacctcccggttcccccggtccaagaagcagcggatcctgctgcagaagttcgacgaggacggcttcaaggagtgcaacctgaacgacacccggtacgtgaaccggttcctgtgccagttcgtggccgaccacatcctgctgaccggcaagggcaagcggcgggtgttcgcctccaacggccagatcaccaacctgctgcggggcttctggggcctgcggaaggtgcgggccgagaacgaccggcaccacgccctggacgccgtggtggtggcctgctccaccgtggccatgcagcagaagatcacccggttcgtgcggtacaaggagatgaacgccttcgacggcaagaccatcgacaaggagaccggcaaggtgctgcaccagaagacccacttcccccagccctgggagttcttcgcccaggaggtgatgatccgggtgttcggcaagcccgacggcaagcccgagttcgaggaggccgacacccccgagaagctgcggaccctgctggccgagaagctgtcctcccggcccgaggccgtgcacgagtacgtgacccccctgttcgtgtcccgggcccccaaccggaagatgtccggcgcccacaaggacaccctgcggtccgccaagcggttcgtgaagcacaacgagaagatctccgtgaagcgggtgtggctgaccgagatcaagctggccgacctggagaacatggtgaactacaagaacggccgggagatcgagctgtacgaggccctgaaggcccggctggaggcctacggcggcaacgccaagcaggccttcgaccccaaggacaaccccttctacaagaagggcggccagctggtgaaggccgtgcgggtggagaagacccaggagtccggcgtgctgctgaacaagaagaacgcctacaccatcgccgacaacggcgacatggtgcgggtggacgtgttctgcaaggtggacaagaagggcaagaaccagtacttcatcgtgcccatctacgcctggcaggtggccgagaacatcctgcccgacatcgactgcaagggctaccggatcgacgactcctacaccttctgcttctccctgcacaagtacgacctgatcgccttccagaaggacgagaagtccaaggtggagttcgcctactacatcaactgcgactcctccaacggccggttctacctggcctggcacgacaagggctccaaggagcagcagttccggatctccacccagaacctggtgctgatccagaagtaccaggtgaacgagctgggcaaggagatccggccctgccggctgaagaagcggccccccgtgcggtccgagtccgccacccccgagtccgtgtccggctggcggctgttcaagaagatctcctagctagcaccagcctcaagaacacccgaatggagtctctaagctacataataccaacttacactttacaaaatgttgtcccccaaaatgtagccattcgtatctgctcctaataaaaagaaagtttcttcacattctctcgagaaaaaaaaaaaatggaaaaaaaaaaaacggaaaaaaaaaaaaggtaaaaaaaaaaaatataaaaaaaaaaaacataaaaaaaaaaaacgaaaaaaaaaaaacgtaaaaaaaaaaaactcaaaaaaaaaaaagataaaaaaaaaaaacctaaaaaaaaaaaatgtaaaaaaaaaaaagggaaaaaaaaaaaacgcaaaaaaaaaaaacacaaaaaaaaaaaatgcaaaaaaaaaaaatcgaaaaaaaaaaaatctaaaaaaaaaaaacgaaaaaaaaaaaacccaaaaaaaaaaaagacaaaaaaaaaaaatagaaaaaaaaaaaagttaaaaaaaaaaaactgaaaaaaaaaaaatttaaaaaaaaaaaatctag

SEQ ID NO: 366 - RNA, 3933 nt, synthetic construct
  misc_feature 1..3933: note = Synthetic
  source 1..3933: mol_type = other RNA
gggaagctcagaataaacgctcaactttggccggatctgccaccatggacggctccggcggcggctcccccaagaagaagcggaaggtgggcggctccggcggcggcgccgccttcaagcccaaccccatcaactacatcctgggcctggacatcggcatcgcctccgtgggctgggccatggtggagatcgacgaggaggagaaccccatccggctgatcgacctgggcgtgcgggtgttcgagcgggccgaggtgcccaagaccggcgactccctggccatggcccggcggctggcccggtccgtgcggcggctgacccggcggcgggcccaccggctgctgcgggcccggcggctgctgaagcgggagggcgtgctgcaggccgccgacttcgacgagaacggcctgatcaagtccctgcccaacaccccctggcagctgcgggccgccgccctggaccggaagctgacccccctggagtggtccgccgtgctgctgcacctgatcaagcaccggggctacctgtcccagcggaagaacgagggcgagaccgccgacaaggagctgggcgccctgctgaagggcgtggccaacaacgcccacgccctgcagaccggcgacttccggacccccgccgagctggccctgaacaagttcgagaaggagtccggccacatccggaaccagcggggcgactactcccacaccttctcccggaaggacctgcaggccgagctgatcctgctgttcgagaagcagaaggagttcggcaacccccacgtgtccggcggcctgaaggagggcatcgagaccctgctgatgacccagcggcccgccctgtccggcgacgccgtgcagaagatgctgggccactgcaccttcgagcccgccgagcccaaggccgccaagaacacctacaccgccgagcggttcatctggctgaccaagctgaacaacctgcggatcctggagcagggctccgagcggcccctgaccgacaccgagcgggccaccctgatggacgagccctaccggaagtccaagctgacctacgcccaggcccggaagctgctgggcctggaggacaccgccttcttcaagggcctgcggtacggcaaggacaacgccgaggcctccaccctgatggagatgaaggcctaccacgccatctcccgggccctggagaaggagggcctgaaggacaagaagtcccccctgaacctgtcctccgagctgcaggacgagatcggcaccgccttctccctgttcaagaccgacgaggacatcaccggccggctgaaggaccgggtgcagcccgagatcctggaggccctgctgaagcacatctccttcgacaagttcgtgcagatctccctgaaggccctgcggcggatcgtgcccctgatggagcagggcaagcggtacgacgaggcctgcgccgagatctacggcgaccactacggcaagaagaacaccgaggagaagatctacctgccccccatccccgccgacgagatccggaaccccgtggtgctgcgggccctgtcccaggcccggaaggtgatcaacggcgtggtgcggcggtacggctcccccgcccggatccacatcgagaccgcccgggaggtgggcaagtccttcaaggaccggaaggagatcgagaagcggcaggaggagaaccggaaggaccgggagaaggccgccgccaagttccgggagtacttccccaacttcgtgggcgagcccaagtccaaggacatcctgaagctgcggctgtacgagcagcagcacggcaagtgcctgtactccggcaaggagatcaacctggtgcggctgaacgagaagggctacgtggagatcgaccacgccctgcccttctcccggacctgggacgactccttcaacaacaaggtgctggtgctgggctccgagaaccagaacaagggcaaccagaccccctacgagtacttcaacggcaaggacaactcccgggagtggcaggagttcaaggcccgggtggagacctcccggttcccccggtccaagaagcagcggatcctgctgcagaagttcgacgaggacggcttcaaggagtgcaacctgaacgacacccggtacgtgaaccggttcctgtgccagttcgtggccgaccacatcctgctgaccggcaagggcaagcggcgggtgttcgcctccaacggccagatcaccaacctgctgcggggcttctggggcctgcggaaggtgcgggccgagaacgaccggcaccacgccctggacgccgtggtggtggcctgctccaccgtggccatgcagcagaagatcacccggttcgtgcggtacaaggagatgaacgccttcgacggcaagaccatcgacaaggagaccggcaaggtgctgcaccagaagacccacttcccccagccctgggagttcttcgcccaggaggtgatgatccgggtgttcggcaagcccgacggcaagcccgagttcgaggaggccgacacccccgagaagctgcggaccctgctggccgagaagctgtcctcccggcccgaggccgtgcacgagtacgtgacccccctgttcgtgtcccgggcccccaaccggaagatgtccggcgcccacaaggacaccctgcggtccgccaagcggttcgtgaagcacaacgagaagatctccgtgaagcgggtgtggctgaccgagatcaagctggccgacctggagaacatggtgaactacaagaacggccgggagatcgagctgtacgaggccctgaaggcccggctggaggcctacggcggcaacgccaagcaggccttcgaccccaaggacaaccccttctacaagaagggcggccagctggtgaaggccgtgcgggtggagaagacccaggagtccggcgtgctgctgaacaagaagaacgcctacaccatcgccgacaacggcgacatggtgcgggtggacgtgttctgcaaggtggacaagaagggcaagaaccagtacttcatcgtgcccatctacgcctggcaggtggccgagaacatcctgcccgacatcgactgcaagggctaccggatcgacgactcctacaccttctgcttctccctgcacaagtacgacctgatcgccttccagaaggacgagaagtccaaggtggagttcgcctactacatcaactgcgactcctccaacggccggttctacctggcctggcacgacaagggctccaaggagcagcagttccggatctccacccagaacctggtgctgatccagaagtaccaggtgaacgagctgggcaaggagatccggccctgccggctgaagaagcggccccccgtgcggtccggaaagcggaccgccgacggctccggaggaggaagccccgccgccaagaagaagaagctggactagctagcaccagcctcaagaacacccgaatggagtctctaagctacataataccaacttacactttacaaaatgttgtcccccaaaatgtagccattcgtatctgctcctaataaaaagaaagtttcttcacattctctcgagaaaaaaaaaaaatggaaaaaaaaaaaacggaaaaaaaaaaaaggtaaaaaaaaaaaatataaaaaaaaaaaacataaaaaaaaaaaacgaaaaaaaaaaaacgtaaaaaaaaaaaactcaaaaaaaaaaagataaaaaaaaaaaacctaaaaaaaaaaaatgtaaaaaaaaaaaagggaaaaaaaaaaaacgcaaaaaaaaaaaacacaaaaaaaaaaaatgcaaaaaaaaaaaatcgaaaaaaaaaaaatctaaaaaaaaaaaacgaaaaaaaaaaaacccaaaaaaaaaaaagacaaaaaaaaaaaatagaaaaaaaaaaagttaaaaaaaaaaaactgaaaaaaaaaaaatttaaaaaaaaaaaatctag

SEQ ID NO: 367 - RNA, 3921 nt, synthetic construct
  misc_feature 1..3921: note = Synthetic
  source 1..3921: mol_type = other RNA
gggaagctcagaataaacgctcaactttggccggatctgccaccatggacggctccggcggcggctcccccaagaagaagcggaaggtggaggacaagcggcccgccgccaccaagaaggccggccaggccaagaagaagaagggcggctccggcggcggcgccgccttcaagcccaaccccatcaactacatcctgggcctggacatcggcatcgcctccgtgggctgggccatggtggagatcgacgaggaggagaaccccatccggctgatcgacctgggcgtgcgggtgttcgagcgggccgaggtgcccaagaccggcgactccctggccatggcccggcggctggcccggtccgtgcggcggctgacccggcggcgggcccaccggctgctgcgggcccggcggctgctgaagcgggagggcgtgctgcaggccgccgacttcgacgagaacggcctgatcaagtccctgcccaacaccccctggcagctgcgggccgccgccctggaccggaagctgacccccctggagtggtccgccgtgctgctgcacctgatcaagcaccggggctacctgtcccagcggaagaacgagggcgagaccgccgacaaggagctgggcgccctgctgaagggcgtggccaacaacgcccacgccctgcagaccggcgacttccggacccccgccgagctggccctgaacaagttcgagaaggagtccggccacatccggaaccagcggggcgactactcccacaccttctcccggaaggacctgcaggccgagctgatcctgctgttcgagaagcagaaggagttcggcaacccccacgtgtccggcggcctgaaggagggcatcgagaccctgctgatgacccagcggcccgccctgtccggcgacgccgtgcagaagatgctgggccactgcaccttcgagcccgccgagcccaaggccgccaagaacacctacaccgccgagcggttcatctggctgaccaagctgaacaacctgcggatcctggagcagggctccgagcggcccctgaccgacaccgagcgggccaccctgatggacgagccctaccggaagtccaagctgacctacgcccaggcccggaagctgctgggcctggaggacaccgccttcttcaagggcctgcggtacggcaaggacaacgccgaggcctccaccctgatggagatgaaggcctaccacgccatctcccgggccctggagaaggagggcctgaaggacaagaagtcccccctgaacctgtcctccgagctgcaggacgagatcggcaccgccttctccctgttcaagaccgacgaggacatcaccggccggctgaaggaccgggtgcagcccgagatcctggaggccctgctgaagcacatctccttcgacaagttcgtgcagatctccctgaaggccctgcggcggatcgtgcccctgatggagcagggcaagcggtacgacgaggcctgcgccgagatctacggcgaccactacggcaagaagaacaccgaggagaagatctacctgccccccatccccgccgacgagatccggaaccccgtggtgctgcgggccctgtcccaggcccggaaggtgatcaacggcgtggtgcggcggtacggctcccccgcccggatccacatcgagaccgcccgggaggtgggcaagtccttcaaggaccggaaggagatcgagaagcggcaggaggagaaccggaaggaccgggagaaggccgccgccaagttccgggagtacttccccaacttcgtgggcgagcccaagtccaaggacatcctgaagctgcggctgtacgagcagcagcacggcaagtgcctgtactccggcaaggagatcaacctggtgcggctgaacgagaagggctacgtggagatcgaccacgccctgcccttctcccggacctgggacgactccttcaacaacaaggtgctggtgctgggctccgagaaccagaacaagggcaaccagaccccctacgagtacttcaacggcaaggacaactcccgggagtggcaggagttcaaggcccgggtggagacctcccggttcccccggtccaagaagcagcggatcctgctgcagaagttcgacgaggacggcttcaaggagtgcaacctgaacgacacccggtacgtgaaccggttcctgtgccagttcgtggccgaccacatcctgctgaccggcaagggcaagcggcgggtgttcgcctccaacggccagatcaccaacctgctgcggggcttctggggcctgcggaaggtgcgggccgagaacgaccggcaccacgccctggacgccgtggtggtggcctgctccaccgtggccatgcagcagaagatcacccggttcgtgcggtacaaggagatgaacgccttcgacggcaagaccatcgacaaggagaccggcaaggtgctgcaccagaagacccacttcccccagccctgggagttcttcgcccaggaggtgatgatccgggtgttcggcaagcccgacggcaagcccgagttcgaggaggccgacacccccgagaagctgcggaccctgctggccgagaagctgtcctcccggcccgaggccgtgcacgagtacgtgacccccctgttcgtgtcccgggcccccaaccggaagatgtccggcgcccacaaggacaccctgcggtccgccaagcggttcgtgaagcacaacgagaagatctccgtgaagcgggtgtggctgaccgagatcaagctggccgacctggagaacatggtgaactacaagaacggccgggagatcgagctgtacgaggccctgaaggcccggctggaggcctacggcggcaacgccaagcaggccttcgaccccaaggacaaccccttctacaagaagggcggccagctggtgaaggccgtgcgggtggagaagacccaggagtccggcgtgctgctgaacaagaagaacgcctacaccatcgccgacaacggcgacatggtgcgggtggacgtgttctgcaaggtggacaagaagggcaagaaccagtacttcatcgtgcccatctacgcctggcaggtggccgagaacatcctgcccgacatcgactgcaagggctaccggatcgacgactcctacaccttctgcttctccctgcacaagtacgacctgatcgccttccagaaggacgagaagtccaaggtggagttcgcctactacatcaactgcgactcctccaacggccggttctacctggcctggcacgacaagggctccaaggagcagcagttccggatctccacccagaacctggtgctgatccagaagtaccaggtgaacgagctgggcaaggagatccggccctgccggctgaagaagcggccccccgtgcggtagctagcaccagcctcaagaacacccgaatggagtctctaagctacataataccaacttacactttacaaaatgttgtcccccaaaatgtagccattcgtatctgctcctaataaaaagaaagtttcttcacattctctcgagaaaaaaaaaaaatggaaaaaaaaaaaacggaaaaaaaaaaaaggtaaaaaaaaaaaatataaaaaaaaaaaacataaaaaaaaaaaacgaaaaaaaaaaaacgtaaaaaaaaaaaactcaaaaaaaaaaagataaaaaaaaaaaacctaaaaaaaaaaaatgtaaaaaaaaaaaagggaaaaaaaaaaaacgcaaaaaaaaaaaacacaaaaaaaaaaaatgcaaaaaaaaaaaatcgaaaaaaaaaaaatctaaaaaaaaaaaacgaaaaaaaaaaaacccaaaaaaaaaaaagacaaaaaaaaaaaatagaaaaaaaaaaagttaaaaaaaaaaaactgaaaaaaaaaaaatttaaaaaaaaaaaatctag

SEQ ID NO: 368 - RNA, 3980 nt, synthetic construct
  misc_feature 1..3980: note = Synthetic
  source 1..3980: mol_type = other RNA
gggaagctcagaataaacgctcaactttggccggatctgccaccatggacggctccggcggcggctcccccaagaagaagcggaaggtggaggacaagcggcccgccgccaccaagaaggccggccaggccaagaagaagaagggcggctccggcggcggcgccgccttcaagcccaaccccatcaactacatcctgggcctggacatcggcatcgcctccgtgggctgggccatggtggagatcgacgaggaggagaaccccatccggctgatcgacctgggcgtgcgggtgttcgagcgggccgaggtgcccaagaccggcgactccctggccatggcccggcggctggcccggtccgtgcggcggctgacccggcggcgggcccaccggctgctgcgggcccggcggctgctgaagcgggagggcgtgctgcaggccgccgacttcgacgagaacggcctgatcaagtccctgcccaacaccccctggcagctgcgggccgccgccctggaccggaagctgacccccctggagtggtccgccgtgctgctgcacctgatcaagcaccggggctacctgtcccagcggaagaacgagggcgagaccgccgacaaggagctgggcgccctgctgaagggcgtggccaacaacgcccacgccctgcagaccggcgacttccggacccccgccgagctggccctgaacaagttcgagaaggagtccggccacatccggaaccagcggggcgactactcccacaccttctcccggaaggacctgcaggccgagctgatcctgctgttcgagaagcagaaggagttcggcaacccccacgtgtccggcggcctgaaggagggcatcgagaccctgctgatgacccagcggcccgccctgtccggcgacgccgtgcagaagatgctgggccactgcaccttcgagcccgccgagcccaaggccgccaagaacacctacaccgccgagcggttcatctggctgaccaagctgaacaacctgcggatcctggagcagggctccgagcggcccctgaccgacaccgagcgggccaccctgatggacgagccctaccggaagtccaagctgacctacgcccaggcccggaagctgctgggcctggaggacaccgccttcttcaagggcctgcggtacggcaaggacaacgccgaggcctccaccctgatggagatgaaggcctaccacgccatctcccgggccctggagaaggagggcctgaaggacaagaagtcccccctgaacctgtcctccgagctgcaggacgagatcggcaccgccttctccctgttcaagaccgacgaggacatcaccggccggctgaaggaccgggtgcagcccgagatcctggaggccctgctgaagcacatctccttcgacaagttcgtgcagatctccctgaaggccctgcggcggatcgtgcccctgatggagcagggcaagcggtacgacgaggcctgcgccgagatctacggcgaccactacggcaagaagaacaccgaggagaagatctacctgccccccatccccgccgacgagatccggaaccccgtggtgctgcgggccctgtcccaggcccggaaggtgatcaacggcgtggtgcggcggtacggctcccccgcccggatccacatcgagaccgcccgggaggtgggcaagtccttcaaggaccggaaggagatcgagaagcggcaggaggagaaccggaaggaccgggagaaggccgccgccaagttccgggagtacttccccaacttcgtgggcgagcccaagtccaaggacatcctgaagctgcggctgtacgagcagcagcacggcaagtgcctgtactccggcaaggagatcaacctggtgcggctgaacgagaagggctacgtggagatcgaccacgccctgcccttctcccggacctgggacgactccttcaacaacaaggtgctggtgctgggctccgagaaccagaacaagggcaaccagaccccctacgagtacttcaacggcaaggacaactcccgggagtggcaggagttcaaggcccgggtggagacctcccggttcccccggtccaagaagcagcggatcctgctgcagaagttcgacgaggacggcttcaaggagtgcaacctgaacgacacccggtacgtgaaccggttcctgtgccagttcgtggccgaccacatcctgctgaccggcaagggcaagcggcgggtgttcgcctccaacggccagatcaccaacctgctgcggggcttctggggcctgcggaaggtgcgggccgagaacgaccggcaccacgccctggacgccgtggtggtggcctgctccaccgtggccatgcagcagaagatcacccggttcgtgcggtacaaggagatgaacgccttcgacggcaagaccatcgacaaggagaccggcaaggtgctgcaccagaagacccacttcccccagccctgggagttcttcgcccaggaggtgatgatccgggtgttcggcaagcccgacggcaagcccgagttcgaggaggccgacacccccgagaagctgcggaccctgctggccgagaagctgtcctcccggcccgaggccgtgcacgagtacgtgacccccctgttcgtgtcccgggcccccaaccggaagatgtccggcgcccacaaggacaccctgcggtccgccaagcggttcgtgaagcacaacgagaagatctccgtgaagcgggtgtggctgaccgagatcaagctggccgacctggagaacatggtgaactacaagaacggccgggagatcgagctgtacgaggccctgaaggcccggctggaggcctacggcggcaacgccaagcaggccttcgaccccaaggacaaccccttctacaagaagggcggccagctggtgaaggccgtgcgggtggagaagacccaggagtccggcgtgctgctgaacaagaagaacgcctacaccatcgccgacaacggcgacatggtgcgggtggacgtgttctgcaaggtggacaagaagggcaagaaccagtacttcatcgtgcccatctacgcctggcaggtggccgagaacatcctgcccgacatcgactgcaagggctaccggatcgacgactcctacaccttctgcttctccctgcacaagtacgacctgatcgccttccagaaggacgagaagtccaaggtggagttcgcctactacatcaactgcgactcctccaacggccggttctacctggcctggcacgacaagggctccaaggagcagcagttccggatctccacccagaacctggtgctgatccagaagtaccaggtgaacgagctgggcaaggagatccggccctgccggctgaagaagcggccccccgtgcggtccgagtccgccacccccgagtccgtgtccggctggcggctgttcaagaagatctcctagctagcaccagcctcaagaacacccgaatggagtctctaagctacataataccaacttacactttacaaaatgttgtcccccaaaatgtagccattcgtatctgctcctaataaaaagaaagtttcttcacattctctcgagaaaaaaaaaaaatggaaaaaaaaaaaacggaaaaaaaaaaaaggtaaaaaaaaaaaatataaaaaaaaaaaacataaaaaaaaaaaacgaaaaaaaaaaaacgtaaaaaaaaaaaactcaaaaaaaaaaaagataaaaaaaaaaaacctaaaaaaaaaaaatgtaaaaaaaaaaaagggaaaaaaaaaaaacgcaaaaaaaaaaaacacaaaaaaaaaaaatgcaaaaaaaaaaaatcgaaaaaaaaaaaatctaaaaaaaaaaaacgaaaaaaaaaaaacccaaaaaaaaaaaagacaaaaaaaaaaaatagaaaaaaaaaaaagttaaaaaaaaaaaactgaaaaaaaaaaaatttaaaaaaaaaaaatctag

SEQ ID NO: 369 - RNA, 3867 nt, synthetic construct
  misc_feature 1..3867: note = Synthetic
  source 1..3867: mol_type = other RNA
gggaagctcagaataaacgctcaactttggccggatctgccaccatggacggctccggcggcggctcccccaagaagaagcggaaggtgggcggctccggcggcggcgccgccttcaagcccaaccccatcaactacatcctgggcctggacatcggcatcgcctccgtgggctgggccatggtggagatcgacgaggaggagaaccccatccggctgatcgacctgggcgtgcgggtgttcgagcgggccgaggtgcccaagaccggcgactccctggccatggcccggcggctggcccggtccgtgcggcggctgacccggcggcgggcccaccggctgctgcgggcccggcggctgctgaagcgggagggcgtgctgcaggccgccgacttcgacgagaacggcctgatcaagtccctgcccaacaccccctggcagctgcgggccgccgccctggaccggaagctgacccccctggagtggtccgccgtgctgctgcacctgatcaagcaccggggctacctgtcccagcggaagaacgagggcgagaccgccgacaaggagctgggcgccctgctgaagggcgtggccaacaacgcccacgccctgcagaccggcgacttccggacccccgccgagctggccctgaacaagttcgagaaggagtccggccacatccggaaccagcggggcgactactcccacaccttctcccggaaggacctgcaggccgagctgatcctgctgttcgagaagcagaaggagttcggcaacccccacgtgtccggcggcctgaaggagggcatcgagaccctgctgatgacccagcggcccgccctgtccggcgacgccgtgcagaagatgctgggccactgcaccttcgagcccgccgagcccaaggccgccaagaacacctacaccgccgagcggttcatctggctgaccaagctgaacaacctgcggatcctggagcagggctccgagcggcccctgaccgacaccgagcgggccaccctgatggacgagccctaccggaagtccaagctgacctacgcccaggcccggaagctgctgggcctggaggacaccgccttcttcaagggcctgcggtacggcaaggacaacgccgaggcctccaccctgatggagatgaaggcctaccacgccatctcccgggccctggagaaggagggcctgaaggacaagaagtcccccctgaacctgtcctccgagctgcaggacgagatcggcaccgccttctccctgttcaagaccgacgaggacatcaccggccggctgaaggaccgggtgcagcccgagatcctggaggccctgctgaagcacatctccttcgacaagttcgtgcagatctccctgaaggccctgcggcggatcgtgcccctgatggagcagggcaagcggtacgacgaggcctgcgccgagatctacggcgaccactacggcaagaagaacaccgaggagaagatctacctgccccccatccccgccgacgagatccggaaccccgtggtgctgcgggccctgtcccaggcccggaaggtgatcaacggcgtggtgcggcggtacggctcccccgcccggatccacatcgagaccgcccgggaggtgggcaagtccttcaaggaccggaaggagatcgagaagcggcaggaggagaaccggaaggaccgggagaaggccgccgccaagttccgggagtacttccccaacttcgtgggcgagcccaagtccaaggacatcctgaagctgcggctgtacgagcagcagcacggcaagtgcctgtactccggcaaggagatcaacctggtgcggctgaacgagaagggctacgtggagatcgaccacgccctgcccttctcccggacctgggacgactccttcaacaacaaggtgctggtgctgggctccgagaaccagaacaagggcaaccagaccccctacgagtacttcaacggcaaggacaactcccgggagtggcaggagttcaaggcccgggtggagacctcccggttcccccggtccaagaagcagcggatcctgctgcagaagttcgacgaggacggcttcaaggagtgcaacctgaacgacacccggtacgtgaaccggttcctgtgccagttcgtggccgaccacatcctgctgaccggcaagggcaagcggcgggtgttcgcctccaacggccagatcaccaacctgctgcggggcttctggggcctgcggaaggtgcgggccgagaacgaccggcaccacgccctggacgccgtggtggtggcctgctccaccgtggccatgcagcagaagatcacccggttcgtgcggtacaaggagatgaacgccttcgacggcaagaccatcgacaaggagaccggcaaggtgctgcaccagaagacccacttcccccagccctgggagttcttcgcccaggaggtgatgatccgggtgttcggcaagcccgacggcaagcccgagttcgaggaggccgacacccccgagaagctgcggaccctgctggccgagaagctgtcctcccggcccgaggccgtgcacgagtacgtgacccccctgttcgtgtcccgggcccccaaccggaagatgtccggcgcccacaaggacaccctgcggtccgccaagcggttcgtgaagcacaacgagaagatctccgtgaagcgggtgtggctgaccgagatcaagctggccgacctggagaacatggtgaactacaagaacggccgggagatcgagctgtacgaggccctgaaggcccggctggaggcctacggcggcaacgccaagcaggccttcgaccccaaggacaaccccttctacaagaagggcggccagctggtgaaggccgtgcgggtggagaagacccaggagtccggcgtgctgctgaacaagaagaacgcctacaccatcgccgacaacggcgacatggtgcgggtggacgtgttctgcaaggtggacaagaagggcaagaaccagtacttcatcgtgcccatctacgcctggcaggtggccgagaacatcctgcccgacatcgactgcaagggctaccggatcgacgactcctacaccttctgcttctccctgcacaagtacgacctgatcgccttccagaaggacgagaagtccaaggtggagttcgcctactacatcaactgcgactcctccaacggccggttctacctggcctggcacgacaagggctccaaggagcagcagttccggatctccacccagaacctggtgctgatccagaagtaccaggtgaacgagctgggcaaggagatccggccctgccggctgaagaagcggccccccgtgcggtagctagcaccagcctcaagaacacccgaatggagtctctaagctacataataccaacttacactttacaaaatgttgtcccccaaaatgtagccattcgtatctgctcctaataaaaagaaagtttcttcacattctctcgagaaaaaaaaaaaatggaaaaaaaaaaaacggaaaaaaaaaaaaggtaaaaaaaaaaaatataaaaaaaaaaaacataaaaaaaaaaaacgaaaaaaaaaaaacgtaaaaaaaaaaaactcaaaaaaaaaaagataaaaaaaaaaaacctaaaaaaaaaaaatgtaaaaaaaaaaaagggaaaaaaaaaaaacgcaaaaaaaaaaaacacaaaaaaaaaaaatgcaaaaaaaaaaaatcgaaaaaaaaaaaatctaaaaaaaaaaaacgaaaaaaaaaaaacccaaaaaaaaaaaagacaaaaaaaaaaaatagaaaaaaaaaaagttaaaaaaaaaaaactgaaaaaaaaaaaatttaaaaaaaaaaaatctag

SEQ ID NO: 370 - RNA, 4563 nt, synthetic construct
  misc_feature 1..4563: note = Synthetic
  source 1..4563: mol_type = other RNA
gggaagctcagaataaacgctcaactttggccggatctgccaccatggacggctccggcggcggctcccccaagaagaagcggaaggtggaggacaagcggcccgccgccaccaagaaggccggccaggccaagaagaagaagggcggctccggcggcggcgaggcctcccccgcctccggcccccggcacctgatggacccccacatcttcacctccaacttcaacaacggcatcggccggcacaagacctacctgtgctacgaggtggagcggctggacaacggcacctccgtgaagatggaccagcaccggggcttcctgcacaaccaggccaagaacctgctgtgcggcttctacggccggcacgccgagctgcggttcctggacctggtgccctccctgcagctggaccccgcccagatctaccgggtgacctggttcatctcctggtccccctgcttctcctggggctgcgccggcgaggtgcgggccttcctgcaggagaacacccacgtgcggctgcggatcttcgccgcccggatctacgactacgaccccctgtacaaggaggccctgcagatgctgcgggacgccggcgcccaggtgtccatcatgacctacgacgagttcaagcactgctgggacaccttcgtggaccaccagggctgccccttccagccctgggacggcctggacgagcactcccaggccctgtccggccggctgcgggccatcctgcagaaccagggcaactccggctccgagacccccggcacctccgagtccgccacccccgagtccgcagcgttcaaaccaaatcccatcaactacatcctgggcctggccatcggcatcgcctccgtgggctgggccatggtggagatcgacgaggaggagaaccccatccggctgatcgacctgggcgtgcgggtgttcgagcgggccgaggtgcccaagaccggcgactccctggccatggcccggcggctggcccggtccgtgcggcggctgacccggcggcgggcccaccggctgctgcgggcccggcggctgctgaagcgggagggcgtgctgcaggccgccgacttcgacgagaacggcctgatcaagtccctgcccaacaccccctggcagctgcgggccgccgccctggaccggaagctgacccccctggagtggtccgccgtgctgctgcacctgatcaagcaccggggctacctgtcccagcggaagaacgagggcgagaccgccgacaaggagctgggcgccctgctgaagggcgtggccaacaacgcccacgccctgcagaccggcgacttccggacccccgccgagctggccctgaacaagttcgagaaggagtccggccacatccggaaccagcggggcgactactcccacaccttctcccggaaggacctgcaggccgagctgatcctgctgttcgagaagcagaaggagttcggcaacccccacgtgtccggcggcctgaaggagggcatcgagaccctgctgatgacccagcggcccgccctgtccggcgacgccgtgcagaagatgctgggccactgcaccttcgagcccgccgagcccaaggccgccaagaacacctacaccgccgagcggttcatctggctgaccaagctgaacaacctgcggatcctggagcagggctccgagcggcccctgaccgacaccgagcgggccaccctgatggacgagccctaccggaagtccaagctgacctacgcccaggcccggaagctgctgggcctggaggacaccgccttcttcaagggcctgcggtacggcaaggacaacgccgaggcctccaccctgatggagatgaaggcctaccacgccatctcccgggccctggagaaggagggcctgaaggacaagaagtcccccctgaacctgtcctccgagctgcaggacgagatcggcaccgccttctccctgttcaagaccgacgaggacatcaccggccggctgaaggaccgggtgcagcccgagatcctggaggccctgctgaagcacatctccttcgacaagttcgtgcagatctccctgaaggccctgcggcggatcgtgcccctgatggagcagggcaagcggtacgacgaggcctgcgccgagatctacggcgaccactacggcaagaagaacaccgaggagaagatctacctgccccccatccccgccgacgagatccggaaccccgtggtgctgcgggccctgtcccaggcccggaaggtgatcaacggcgtggtgcggcggtacggctcccccgcccggatccacatcgagaccgcccgggaggtgggcaagtccttcaaggaccggaaggagatcgagaagcggcaggaggagaaccggaaggaccgggagaaggccgccgccaagttccgggagtacttccccaacttcgtgggcgagcccaagtccaaggacatcctgaagctgcggctgtacgagcagcagcacggcaagtgcctgtactccggcaaggagatcaacctggtgcggctgaacgagaagggctacgtggagatcgaccacgccctgcccttctcccggacctgggacgactccttcaacaacaaggtgctggtgctgggctccgagaaccagaacaagggcaaccagaccccctacgagtacttcaacggcaaggacaactcccgggagtggcaggagttcaaggcccgggtggagacctcccggttcccccggtccaagaagcagcggatcctgctgcagaagttcgacgaggacggcttcaaggagtgcaacctgaacgacacccggtacgtgaaccgcttcctgtgccagttcgtggccgaccacatcctgctgaccggcaagggcaagcggcgggtgttcgcctccaacggccagatcaccaacctgctgcggggcttctggggcctgcggaaggtgcgggccgagaacgaccggcaccacgccctggacgccgtggtggtggcctgctccaccgtggccatgcagcagaagatcacccggttcgtgcggtacaaggagatgaacgccttcgacggcaagaccatcgacaaggagaccggcaaggtgctgcaccagaagacccacttcccccagccctgggagttcttcgcccaggaggtgatgatccgggtgttcggcaagcccgacggcaagcccgagttcgaggaggccgacacccccgagaagctgcggaccctgctggccgagaagctgtcctcccggcccgaggccgtgcacgagtacgtgacccccctgttcgtgtcccgggcccccaaccggaagatgtccggcgcccacaaggacaccctgcggtccgccaagcggttcgtgaagcacaacgagaagatctccgtgaagcgggtgtggctgaccgagatcaagctggccgacctggagaacatggtgaactacaagaacggccgggagatcgagctgtacgaggccctgaaggcccggctggaggcctacggcggcaacgccaagcaggccttcgaccccaaggacaaccccttctacaagaagggcggccagctggtgaaggccgtgcgggtggagaagacccaggagtccggcgtgctgctgaacaagaagaacgcctacaccatcgccgacaacggcgacatggtgcgggtggacgtgttctgcaaggtggacaagaagggcaagaaccagtacttcatcgtgcccatctacgcctggcaggtggccgagaacatcctgcccgacatcgactgcaagggctaccggatcgacgactcctacaccttctgcttctccctgcacaagtacgacctgatcgccttccagaaggacgagaagtccaaggtggagttcgcctactacatcaactgcgactcctccaacggccggttctacctggcctggcacgacaagggctccaaggagcagcagttccggatctccacccagaacctggtgctgatccagaagtaccaggtgaacgagctgggcaaggagatccggccctgccggctgaagaagcggccccccgtgcggtagtgactagcaccagcctcaagaacacccgaatggagtctctaagctacataataccaacttacactttacaaaatgttgtcccccaaaatgtagccattcgtatctgctcctaataaaaagaaagtttcttcacattctctcgagaaaaaaaaaaaatggaaaaaaaaaaaacggaaaaaaaaaaaggtaaaaaaaaaaaatataaaaaaaaaaacataaaaaaaaaaaacgaaaaaaaaaaaacgtaaaaaaaaaaaactcaaaaaaaaaaagataaaaaaaaaaaacctaaaaaaaaaaaatgtaaaaaaaaaaaagggaaaaaaaaaaacgcaaaaaaaaaaaacacaaaaaaaaaaaatgcaaaaaaaaaaaatcgaaaaaaaaaaaatctaaaaaaaaaaaacgaaaaaaaaaaaacccaaaaaaaaaaaagacaaaaaaaaaaaatagaaaaaaaaaaagttaaaaaaaaaaaactgaaaaaaaaaaaatttaaaaaaaaaaaatctag

SEQ ID NO: 371 - RNA, 5342 nt, synthetic construct
  misc_feature 1..5342: note = Synthetic
  source 1..5342: mol_type = other RNA
gggaagctcagaataaacgctcaactttggccggatctgccaccatggaggcctcccccgcctccggcccccggcacctgatggacccccacatcttcacctccaacttcaacaacggcatcggccggcacaagacctacctgtgctacgaggtggagcggctggacaacggcacctccgtgaagatggaccagcaccggggcttcctgcacaaccaggccaagaacctgctgtgcggcttctacggccggcacgccgagctgcggttcctggacctggtgccctccctgcagctggaccccgcccagatctaccgggtgacctggttcatctcctggtccccctgcttctcctggggctgcgccggcgaggtgcgggccttcctgcaggagaacacccacgtgcggctgcggatcttcgccgcccggatctacgactacgaccccctgtacaaggaggccctgcagatgctgcgggacgccggcgcccaggtgtccatcatgacctacgacgagttcaagcactgctgggacaccttcgtggaccaccagggctgccccttccagccctgggacggcctggacgagcactcccaggccctgtccggccggctgcgggccatcctgcagaaccagggcaactccggctccgagacccccggcacctccgagtccgccacccccgagtccgacaagaagtactccatcggcctggccatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtgactagcaccagcctcaagaacacccgaatggagtctctaagctacataataccaacttacactttacaaaatgttgtcccccaaaatgtagccattcgtatctgctcctaataaaaagaaagtttcttcacattctctcgagaaaaaaaaaaaatggaaaaaaaaaaaacggaaaaaaaaaaaaggtaaaaaaaaaaaatataaaaaaaaaaaacataaaaaaaaaaaacgaaaaaaaaaaaacgtaaaaaaaaaaaactcaaaaaaaaaaaagataaaaaaaaaaaacctaaaaaaaaaaaatgtaaaaaaaaaaaagggaaaaaaaaaaaacgcaaaaaaaaaaaacacaaaaaaaaaaaatgcaaaaaaaaaaaatcgaaaaaaaaaaaatctaaaaaaaaaaaacgaaaaaaaaaaaacccaaaaaaaaaaaagacaaaaaaaaaaaatagaaaaaaaaaaaagttaaaaaaaaaaaactgaaaaaaaaaaaatttaaaaaaaaaaaatctag

SEQ ID NO: 372 - DNA, 3318 bp, synthetic construct
  misc_feature 1..3318: note = Synthetic
  source 1..3318: mol_type = other DNA
atgaccggtgccgccttcaagcccaaccccatcaactacatcctgggcctggacatcggcatcgcctccgtgggctgggccatggtggagatcgacgaggaggagaaccccatccggctgatcgacctgggcgtgcgggtgttcgagcgggccgaggtgcccaagaccggcgactccctggccatggcccggcggctggcccggtccgtgcggcggctgacccggcggcgggcccaccggctgctgcgggcccggcggctgctgaagcgggagggcgtgctgcaggccgccgacttcgacgagaacggcctgatcaagtccctgcccaacaccccctggcagctgcgggccgccgccctggaccggaagctgacccccctggagtggtccgccgtgctgctgcacctgatcaagcaccggggctacctgtcccagcggaagaacgagggcgagaccgccgacaaggagctgggcgccctgctgaagggcgtggccaacaacgcccacgccctgcagaccggcgacttccggacccccgccgagctggccctgaacaagttcgagaaggagtccggccacatccggaaccagcggggcgactactcccacaccttctcccggaaggacctgcaggccgagctgatcctgctgttcgagaagcagaaggagttcggcaacccccacgtgtccggcggcctgaaggagggcatcgagaccctgctgatgacccagcggcccgccctgtccggcgacgccgtgcagaagatgctgggccactgcaccttcgagcccgccgagcccaaggccgccaagaacacctacaccgccgagcggttcatctggctgaccaagctgaacaacctgcggatcctggagcagggctccgagcggcccctgaccgacaccgagcgggccaccctgatggacgagccctaccggaagtccaagctgacctacgcccaggcccggaagctgctgggcctggaggacaccgccttcttcaagggcctgcggtacggcaaggacaacgccgaggcctccaccctgatggagatgaaggcctaccacgccatctcccgggccctggagaaggagggcctgaaggacaagaagtcccccctgaacctgtcctccgagctgcaggacgagatcggcaccgccttctccctgttcaagaccgacgaggacatcaccggccggctgaaggaccgggtgcagcccgagatcctggaggccctgctgaagcacatctccttcgacaagttcgtgcagatctccctgaaggccctgcggcggatcgtgcccctgatggagcagggcaagcggtacgacgaggcctgcgccgagatctacggcgaccactacggcaagaagaacaccgaggagaagatctacctgccccccatccccgccgacgagatccggaaccccgtggtgctgcgggccctgtcccaggcccggaaggtgatcaacggcgtggtgcggcggtacggctcccccgcccggatccacatcgagaccgcccgggaggtgggcaagtccttcaaggaccggaaggagatcgagaagcggcaggaggagaaccggaaggaccgggagaaggccgccgccaagttccgggagtacttccccaacttcgtgggcgagcccaagtccaaggacatcctgaagctgcggctgtacgagcagcagcacggcaagtgcctgtactccggcaaggagatcaacctggtgcggctgaacgagaagggctacgtggagatcgaccacgccctgcccttctcccggacctgggacgactccttcaacaacaaggtgctggtgctgggctccgagaaccagaacaagggcaaccagaccccctacgagtacttcaacggcaaggacaactcccgggagtggcaggagttcaaggcccgggtggagacctcccggttcccccggtccaagaagcagcggatcctgctgcagaagttcgacgaggacggcttcaaggagtgcaacctgaacgacacccggtacgtgaaccgcttcctgtgccagttcgtggccgaccacatcctgctgaccggcaagggcaagcggcgggtgttcgcctccaacggccagatcaccaacctgctgcggggcttctggggcctgcggaaggtgcgggccgagaacgaccggcaccacgccctggacgccgtggtggtggcctgctccaccgtggccatgcagcagaagatcacccggttcgtgcggtacaaggagatgaacgccttcgacggcaagaccatcgacaaggagaccggcaaggtgctgcaccagaagacccacttcccccagccctgggagttcttcgcccaggaggtgatgatccgggtgttcggcaagcccgacggcaagcccgagttcgaggaggccgacacccccgagaagctgcggaccctgctggccgagaagctgtcctcccggcccgaggccgtgcacgagtacgtgacccccctgttcgtgtcccgggcccccaaccggaagatgtccggcgcccacaaggacaccctgcggtccgccaagcggttcgtgaagcacaacgagaagatctccgtgaagcgggtgtggctgaccgagatcaagctggccgacctggagaacatggtgaactacaagaacggccgggagatcgagctgtacgaggccctgaaggcccggctggaggcctacggcggcaacgccaagcaggccttcgaccccaaggacaaccccttctacaagaagggcggccagctggtgaaggccgtgcgggtggagaagacccaggagtccggcgtgctgctgaacaagaagaacgcctacaccatcgccgacaacggcgacatggtgcgggtggacgtgttctgcaaggtggacaagaagggcaagaaccagtacttcatcgtgcccatctacgcctggcaggtggccgagaacatcctgcccgacatcgactgcaagggctaccggatcgacgactcctacaccttctgcttctccctgcacaagtacgacctgatcgccttccagaaggacgagaagtccaaggtggagttcgcctactacatcaactgcgactcctccaacggccggttctacctggcctggcacgacaagggctccaaggagcagcagttccggatctccacccagaacctggtgctgatccagaagtaccaggtgaacgagctgggcaaggagatccggccctgccggctgaagaagcggccccccgtgcggtccggaaagcggaccgccgacggctccgagttcgagtcccccaagaagaagcggaaggtggagtag

SEQ ID NO: 373 - DNA, 327 bp, synthetic construct
  misc_feature 1..327: note = Synthetic
  source 1..327: mol_type = other DNA
atgaccaacctgtccgacatcatcgagaaggagaccggcaagcagctggtgatccaggagtccatcctgatgctgcccgaggaggtggaggaggtgatcggcaacaagcccgagtccgacatcctggtgcacaccgcctacgacgagtccaccgacgagaacgtgatgctgctgacctccgacgcccccgagtacaagccctgggccctggtgatccaggactccaacggcgagaacaagatcaagatgctgtccggcggctccaagcggaccgccgacggctccgagttcgagtcccccaagaagaagcggaaggtggagtgatag

SEQ ID NO: 374 - DNA, 3333 bp, synthetic construct
  misc_feature 1..3333: note = Synthetic
  source 1..3333: mol_type = other DNA
atggtgcccaagaagaagcggaaggtggccgccttcaagcccaaccccatcaactacatcctgggcctggacatcggcatcgcctccgtgggctgggccatggtggagatcgacgaggaggagaaccccatccggctgatcgacctgggcgtgcgggtgttcgagcgggccgaggtgcccaagaccggcgactccctggccatggcccggcggctggcccggtccgtgcggcggctgacccggcggcgggcccaccggctgctgcgggcccggcggctgctgaagcgggagggcgtgctgcaggccgccgacttcgacgagaacggcctgatcaagtccctgcccaacaccccctggcagctgcgggccgccgccctggaccggaagctgacccccctggagtggtccgccgtgctgctgcacctgatcaagcaccggggctacctgtcccagcggaagaacgagggcgagaccgccgacaaggagctgggcgccctgctgaagggcgtggccaacaacgcccacgccctgcagaccggcgacttccggacccccgccgagctggccctgaacaagttcgagaaggagtccggccacatccggaaccagcggggcgactactcccacaccttctcccggaaggacctgcaggccgagctgatcctgctgttcgagaagcagaaggagttcggcaacccccacgtgtccggcggcctgaaggagggcatcgagaccctgctgatgacccagcggcccgccctgtccggcgacgccgtgcagaagatgctgggccactgcaccttcgagcccgccgagcccaaggccgccaagaacacctacaccgccgagcggttcatctggctgaccaagctgaacaacctgcggatcctggagcagggctccgagcggcccctgaccgacaccgagcgggccaccctgatggacgagccctaccggaagtccaagctgacctacgcccaggcccggaagctgctgggcctggaggacaccgccttcttcaagggcctgcggtacggcaaggacaacgccgaggcctccaccctgatggagatgaaggcctaccacgccatctcccgggccctggagaaggagggcctgaaggacaagaagtcccccctgaacctgtcctccgagctgcaggacgagatcggcaccgccttctccctgttcaagaccgacgaggacatcaccggccggctgaaggaccgggtgcagcccgagatcctggaggccctgctgaagcacatctccttcgacaagttcgtgcagatctccctgaaggccctgcggcggatcgtgcccctgatggagcagggcaagcggtacgacgaggcctgcgccgagatctacggcgaccactacggcaagaagaacaccgaggagaagatctacctgccccccatccccgccgacgagatccggaaccccgtggtgctgcgggccctgtcccaggcccggaaggtgatcaacggcgtggtgcggcggtacggctcccccgcccggatccacatcgagaccgcccgggaggtgggcaagtccttcaaggaccggaaggagatcgagaagcggcaggaggagaaccggaaggaccgggagaaggccgccgccaagttccgggagtacttccccaacttcgtgggcgagcccaagtccaaggacatcctgaagctgcggctgtacgagcagcagcacggcaagtgcctgtactccggcaaggagatcaacctggtgcggctgaacgagaagggctacgtggagatcgaccacgccctgcccttctcccggacctgggacgactccttcaacaacaaggtgctggtgctgggctccgagaaccagaacaagggcaaccagaccccctacgagtacttcaacggcaaggacaactcccgggagtggcaggagttcaaggcccgggtggagacctcccggttcccccggtccaagaagcagcggatcctgctgcagaagttcgacgaggacggcttcaaggagtgcaacctgaacgacacccggtacgtgaaccggttcctgtgccagttcgtggccgaccacatcctgctgaccggcaagggcaagcggcgggtgttcgcctccaacggccagatcaccaacctgctgcggggcttctggggcctgcggaaggtgcgggccgagaacgaccggcaccacgccctggacgccgtggtggtggcctgctccaccgtggccatgcagcagaagatcacccggttcgtgcggtacaaggagatgaacgccttcgacggcaagaccatcgacaaggagaccggcaaggtgctgcaccagaagacccacttcccccagccctgggagttcttcgcccaggaggtgatgatccgggtgttcggcaagcccgacggcaagcccgagttcgaggaggccgacacccccgagaagctgcggaccctgctggccgagaagctgtcctcccggcccgaggccgtgcacgagtacgtgacccccctgttcgtgtcccgggcccccaaccggaagatgtccggcgcccacaaggacaccctgcggtccgccaagcggttcgtgaagcacaacgagaagatctccgtgaagcgggtgtggctgaccgagatcaagctggccgacctggagaacatggtgaactacaagaacggccgggagatcgagctgtacgaggccctgaaggcccggctggaggcctacggcggcaacgccaagcaggccttcgaccccaaggacaaccccttctacaagaagggcggccagctggtgaaggccgtgcgggtggagaagacccaggagtccggcgtgctgctgaacaagaagaacgcctacaccatcgccgacaacggcgacatggtgcgggtggacgtgttctgcaaggtggacaagaagggcaagaaccagtacttcatcgtgcccatctacgcctggcaggtggccgagaacatcctgcccgacatcgactgcaagggctaccggatcgacgactcctacaccttctgcttctccctgcacaagtacgacctgatcgccttccagaaggacgagaagtccaaggtggagttcgcctactacatcaactgcgactcctccaacggccggttctacctggcctggcacgacaagggctccaaggagcagcagttccggatctccacccagaacctggtgctgatccagaagtaccaggtgaacgagctgggcaaggagatccggccctgccggctgaagaagcggccccccgtgcggtacccctacgacgtgcccgactacgccgccgcccccgccgccaagaagaagaagctggactag

SEQ ID NO: 375 - DNA, 3246 bp, synthetic construct
  misc_feature 1..3246: note = Synthetic
  source 1..3246: mol_type = other DNA
atggccgccttcaagcccaaccccatcaactacatcctgggcctggacatcggcatcgcctccgtgggctgggccatggtggagatcgacgaggaggagaaccccatccggctgatcgacctgggcgtgcgggtgttcgagcgggccgaggtgcccaagaccggcgactccctggccatggcccggcggctggcccggtccgtgcggcggctgacccggcggcgggcccaccggctgctgcgggcccggcggctgctgaagcgggagggcgtgctgcaggccgccgacttcgacgagaacggcctgatcaagtccctgcccaacaccccctggcagctgcgggccgccgccctggaccggaagctgacccccctggagtggtccgccgtgctgctgcacctgatcaagcaccggggctacctgtcccagcggaagaacgagggcgagaccgccgacaaggagctgggcgccctgctgaagggcgtggccaacaacgcccacgccctgcagaccggcgacttccggacccccgccgagctggccctgaacaagttcgagaaggagtccggccacatccggaaccagcggggcgactactcccacaccttctcccggaaggacctgcaggccgagctgatcctgctgttcgagaagcagaaggagttcggcaacccccacgtgtccggcggcctgaaggagggcatcgagaccctgctgatgacccagcggcccgccctgtccggcgacgccgtgcagaagatgctgggccactgcaccttcgagcccgccgagcccaaggccgccaagaacacctacaccgccgagcggttcatctggctgaccaagctgaacaacctgcggatcctggagcagggctccgagcggcccctgaccgacaccgagcgggccaccctgatggacgagccctaccggaagtccaagctgacctacgcccaggcccggaagctgctgggcctggaggacaccgccttcttcaagggcctgcggtacggcaaggacaacgccgaggcctccaccctgatggagatgaaggcctaccacgccatctcccgggccctggagaaggagggcctgaaggacaagaagtcccccctgaacctgtcctccgagctgcaggacgagatcggcaccgccttctccctgttcaagaccgacgaggacatcaccggccggctgaaggaccgggtgcagcccgagatcctggaggccctgctgaagcacatctccttcgacaagttcgtgcagatctccctgaaggccctgcggcggatcgtgcccctgatggagcagggcaagcggtacgacgaggcctgcgccgagatctacggcgaccactacggcaagaagaacaccgaggagaagatctacctgccccccatccccgccgacgagatccggaaccccgtggtgctgcgggccctgtcccaggcccggaaggtgatcaacggcgtggtgcggcggtacggctcccccgcccggatccacatcgagaccgcccgggaggtgggcaagtccttcaaggaccggaaggagatcgagaagcggcaggaggagaaccggaaggaccgggagaaggccgccgccaagttccgggagtacttccccaacttcgtgggcgagcccaagtccaaggacatcctgaagctgcggctgtacgagcagcagcacggcaagtgcctgtactccggcaaggagatcaacctggtgcggctgaacgagaagggctacgtggagatcgaccacgccctgcccttctcccggacctgggacgactccttcaacaacaaggtgctggtgctgggctccgagaaccagaacaagggcaaccagaccccctacgagtacttcaacggcaaggacaactcccgggagtggcaggagttcaaggcccgggtggagacctcccggttcccccggtccaagaagcagcggatcctgctgcagaagttcgacgaggacggcttcaaggagtgcaacctgaacgacacccggtacgtgaaccggttcctgtgccagttcgtggccgaccacatcctgctgaccggcaagggcaagcggcgggtgttcgcctccaacggccagatcaccaacctgctgcggggcttctggggcctgcggaaggtgcgggccgagaacgaccggcaccacgccctggacgccgtggtggtggcctgctccaccgtggccatgcagcagaagatcacccggttcgtgcggtacaaggagatgaacgccttcgacggcaagaccatcgacaaggagaccggcaaggtgctgcaccagaagacccacttcccccagccctgggagttcttcgcccaggaggtgatgatccgggtgttcggcaagcccgacggcaagcccgagttcgaggaggccgacacccccgagaagctgcggaccctgctggccgagaagctgtcctcccggcccgaggccgtgcacgagtacgtgacccccctgttcgtgtcccgggcccccaaccggaagatgtccggcgcccacaaggacaccctgcggtccgccaagcggttcgtgaagcacaacgagaagatctccgtgaagcgggtgtggctgaccgagatcaagctggccgacctggagaacatggtgaactacaagaacggccgggagatcgagctgtacgaggccctgaaggcccggctggaggcctacggcggcaacgccaagcaggccttcgaccccaaggacaaccccttctacaagaagggcggccagctggtgaaggccgtgcgggtggagaagacccaggagtccggcgtgctgctgaacaagaagaacgcctacaccatcgccgacaacggcgacatggtgcgggtggacgtgttctgcaaggtggacaagaagggcaagaaccagtacttcatcgtgcccatctacgcctggcaggtggccgagaacatcctgcccgacatcgactgcaagggctaccggatcgacgactcctacaccttctgcttctccctgcacaagtacgacctgatcgccttccagaaggacgagaagtccaaggtggagttcgcctactacatcaactgcgactcctccaacggccggttctacctggcctggcacgacaagggctccaaggagcagcagttccggatctccacccagaacctggtgctgatccagaagtaccaggtgaacgagctgggcaaggagatccggccctgccggctgaagaagcggccccccgtgcgg

SEQ ID NO: 376 - DNA, 3366 bp, synthetic construct
  misc_feature 1..3366: note = Synthetic
  source 1..3366: mol_type = other DNA
atggacggctccggcggcggctcccccaagaagaagcggaaggtgggcggctccggcggcggcgccgccttcaagcccaaccccatcaactacatcctgggcctggacatcggcatcgcctccgtgggctgggccatggtggagatcgacgaggaggagaaccccatccggctgatcgacctgggcgtgcgggtgttcgagcgggccgaggtgcccaagaccggcgactccctggccatggcccggcggctggcccggtccgtgcggcggctgacccggcggcgggcccaccggctgctgcgggcccggcggctgctgaagcgggagggcgtgctgcaggccgccgacttcgacgagaacggcctgatcaagtccctgcccaacaccccctggcagctgcgggccgccgccctggaccggaagctgacccccctggagtggtccgccgtgctgctgcacctgatcaagcaccggggctacctgtcccagcggaagaacgagggcgagaccgccgacaaggagctgggcgccctgctgaagggcgtggccaacaacgcccacgccctgcagaccggcgacttccggacccccgccgagctggccctgaacaagttcgagaaggagtccggccacatccggaaccagcggggcgactactcccacaccttctcccggaaggacctgcaggccgagctgatcctgctgttcgagaagcagaaggagttcggcaacccccacgtgtccggcggcctgaaggagggcatcgagaccctgctgatgacccagcggcccgccctgtccggcgacgccgtgcagaagatgctgggccactgcaccttcgagcccgccgagcccaaggccgccaagaacacctacaccgccgagcggttcatctggctgaccaagctgaacaacctgcggatcctggagcagggctccgagcggcccctgaccgacaccgagcgggccaccctgatggacgagccctaccggaagtccaagctgacctacgcccaggcccggaagctgctgggcctggaggacaccgccttcttcaagggcctgcggtacggcaaggacaacgccgaggcctccaccctgatggagatgaaggcctaccacgccatctcccgggccctggagaaggagggcctgaaggacaagaagtcccccctgaacctgtcctccgagctgcaggacgagatcggcaccgccttctccctgttcaagaccgacgaggacatcaccggccggctgaaggaccgggtgcagcccgagatcctggaggccctgctgaagcacatctccttcgacaagttcgtgcagatctccctgaaggccctgcggcggatcgtgcccctgatggagcagggcaagcggtacgacgaggcctgcgccgagatctacggcgaccactacggcaagaagaacaccgaggagaagatctacctgccccccatccccgccgacgagatccggaaccccgtggtgctgcgggccctgtcccaggcccggaaggtgatcaacggcgtggtgcggcggtacggctcccccgcccggatccacatcgagaccgcccgggaggtgggcaagtccttcaaggaccggaaggagatcgagaagcggcaggaggagaaccggaaggaccgggagaaggccgccgccaagttccgggagtacttccccaacttcgtgggcgagcccaagtccaaggacatcctgaagctgcggctgtacgagcagcagcacggcaagtgcctgtactccggcaaggagatcaacctggtgcggctgaacgagaagggctacgtggagatcgaccacgccctgcccttctcccggacctgggacgactccttcaacaacaaggtgctggtgctgggctccgagaaccagaacaagggcaaccagaccccctacgagtacttcaacggcaaggacaactcccgggagtggcaggagttcaaggcccgggtggagacctcccggttcccccggtccaagaagcagcggatcctgctgcagaagttcgacgaggacggcttcaaggagtgcaacctgaacgacacccggtacgtgaaccggttcctgtgccagttcgtggccgaccacatcctgctgaccggcaagggcaagcggcgggtgttcgcctccaacggccagatcaccaacctgctgcggggcttctggggcctgcggaaggtgcgggccgagaacgaccggcaccacgccctggacgccgtggtggtggcctgctccaccgtggccatgcagcagaagatcacccggttcgtgcggtacaaggagatgaacgccttcgacggcaagaccatcgacaaggagaccggcaaggtgctgcaccagaagacccacttcccccagccctgggagttcttcgcccaggaggtgatgatccgggtgttcggcaagcccgacggcaagcccgagttcgaggaggccgacacccccgagaagctgcggaccctgctggccgagaagctgtcctcccggcccgaggccgtgcacgagtacgtgacccccctgttcgtgtcccgggcccccaaccggaagatgtccggcgcccacaaggacaccctgcggtccgccaagcggttcgtgaagcacaacgagaagatctccgtgaagcgggtgtggctgaccgagatcaagctggccgacctggagaacatggtgaactacaagaacggccgggagatcgagctgtacgaggccctgaaggcccggctggaggcctacggcggcaacgccaagcaggccttcgaccccaaggacaaccccttctacaagaagggcggccagctggtgaaggccgtgcgggtggagaagacccaggagtccggcgtgctgctgaacaagaagaacgcctacaccatcgccgacaacggcgacatggtgcgggtggacgtgttctgcaaggtggacaagaagggcaagaaccagtacttcatcgtgcccatctacgcctggcaggtggccgagaacatcctgcccgacatcgactgcaagggctaccggatcgacgactcctacaccttctgcttctccctgcacaagtacgacctgatcgccttccagaaggacgagaagtccaaggtggagttcgcctactacatcaactgcgactcctccaacggccggttctacctggcctggcacgacaagggctccaaggagcagcagttccggatctccacccagaacctggtgctgatccagaagtaccaggtgaacgagctgggcaaggagatccggccctgccggctgaagaagcggccccccgtgcggtccgagtccgccacccccgagtccgtgtccggctggcggctgttcaagaagatctcctag

SEQ ID NO: 377 - DNA, 3375 bp, synthetic construct
  misc_feature 1..3375: note = Synthetic
  source 1..3375: mol_type = other DNA
atggacggctccggcggcggctcccccaagaagaagcggaaggtgggcggctccggcggcggcgccgccttcaagcccaaccccatcaactacatcctgggcctggacatcggcatcgcctccgtgggctgggccatggtggagatcgacgaggaggagaaccccatccggctgatcgacctgggcgtgcgggtgttcgagcgggccgaggtgcccaagaccggcgactccctggccatggcccggcggctggcccggtccgtgcggcggctgacccggcggcgggcccaccggctgctgcgggcccggcggctgctgaagcgggagggcgtgctgcaggccgccgacttcgacgagaacggcctgatcaagtccctgcccaacaccccctggcagctgcgggccgccgccctggaccggaagctgacccccctggagtggtccgccgtgctgctgcacctgatcaagcaccggggctacctgtcccagcggaagaacgagggcgagaccgccgacaaggagctgggcgccctgctgaagggcgtggccaacaacgcccacgccctgcagaccggcgacttccggacccccgccgagctggccctgaacaagttcgagaaggagtccggccacatccggaaccagcggggcgactactcccacaccttctcccggaaggacctgcaggccgagctgatcctgctgttcgagaagcagaaggagttcggcaacccccacgtgtccggcggcctgaaggagggcatcgagaccctgctgatgacccagcggcccgccctgtccggcgacgccgtgcagaagatgctgggccactgcaccttcgagcccgccgagcccaaggccgccaagaacacctacaccgccgagcggttcatctggctgaccaagctgaacaacctgcggatcctggagcagggctccgagcggcccctgaccgacaccgagcgggccaccctgatggacgagccctaccggaagtccaagctgacctacgcccaggcccggaagctgctgggcctggaggacaccgccttcttcaagggcctgcggtacggcaaggacaacgccgaggcctccaccctgatggagatgaaggcctaccacgccatctcccgggccctggagaaggagggcctgaaggacaagaagtcccccctgaacctgtcctccgagctgcaggacgagatcggcaccgccttctccctgttcaagaccgacgaggacatcaccggccggctgaaggaccgggtgcagcccgagatcctggaggccctgctgaagcacatctccttcgacaagttcgtgcagatctccctgaaggccctgcggcggatcgtgcccctgatggagcagggcaagcggtacgacgaggcctgcgccgagatctacggcgaccactacggcaagaagaacaccgaggagaagatctacctgccccccatccccgccgacgagatccggaaccccgtggtgctgcgggccctgtcccaggcccggaaggtgatcaacggcgtggtgcggcggtacggctcccccgcccggatccacatcgagaccgcccgggaggtgggcaagtccttcaaggaccggaaggagatcgagaagcggcaggaggagaaccggaaggaccgggagaaggccgccgccaagttccgggagtacttccccaacttcgtgggcgagcccaagtccaaggacatcctgaagctgcggctgtacgagcagcagcacggcaagtgcctgtactccggcaaggagatcaacctggtgcggctgaacgagaagggctacgtggagatcgaccacgccctgcccttctcccggacctgggacgactccttcaacaacaaggtgctggtgctgggctccgagaaccagaacaagggcaaccagaccccctacgagtacttcaacggcaaggacaactcccgggagtggcaggagttcaaggcccgggtggagacctcccggttcccccggtccaagaagcagcggatcctgctgcagaagttcgacgaggacggcttcaaggagtgcaacctgaacgacacccggtacgtgaaccggttcctgtgccagttcgtggccgaccacatcctgctgaccggcaagggcaagcggcgggtgttcgcctccaacggccagatcaccaacctgctgcggggcttctggggcctgcggaaggtgcgggccgagaacgaccggcaccacgccctggacgccgtggtggtggcctgctccaccgtggccatgcagcagaagatcacccggttcgtgcggtacaaggagatgaacgccttcgacggcaagaccatcgacaaggagaccggcaaggtgctgcaccagaagacccacttcccccagccctgggagttcttcgcccaggaggtgatgatccgggtgttcggcaagcccgacggcaagcccgagttcgaggaggccgacacccccgagaagctgcggaccctgctggccgagaagctgtcctcccggcccgaggccgtgcacgagtacgtgacccccctgttcgtgtcccgggcccccaaccggaagatgtccggcgcccacaaggacaccctgcggtccgccaagcggttcgtgaagcacaacgagaagatctccgtgaagcgggtgtggctgaccgagatcaagctggccgacctggagaacatggtgaactacaagaacggccgggagatcgagctgtacgaggccctgaaggcccggctggaggcctacggcggcaacgccaagcaggccttcgaccccaaggacaaccccttctacaagaagggcggccagctggtgaaggccgtgcgggtggagaagacccaggagtccggcgtgctgctgaacaagaagaacgcctacaccatcgccgacaacggcgacatggtgcgggtggacgtgttctgcaaggtggacaagaagggcaagaaccagtacttcatcgtgcccatctacgcctggcaggtggccgagaacatcctgcccgacatcgactgcaagggctaccggatcgacgactcctacaccttctgcttctccctgcacaagtacgacctgatcgccttccagaaggacgagaagtccaaggtggagttcgcctactacatcaactgcgactcctccaacggccggttctacctggcctggcacgacaagggctccaaggagcagcagttccggatctccacccagaacctggtgctgatccagaagtaccaggtgaacgagctgggcaaggagatccggccctgccggctgaagaagcggccccccgtgcggtccggaaagcggaccgccgacggctccggaggaggaagccccgccgccaagaagaagaagctggactag

SEQ ID NO: 378 - DNA, 3363 bp, synthetic construct
  misc_feature 1..3363: note = Synthetic
  source 1..3363: mol_type = other DNA
atggacggctccggcggcggctcccccaagaagaagcggaaggtggaggacaagcggcccgccgccaccaagaaggccggccaggccaagaagaagaagggcggctccggcggcggcgccgccttcaagcccaaccccatcaactacatcctgggcctggacatcggcatcgcctccgtgggctgggccatggtggagatcgacgaggaggagaaccccatccggctgatcgacctgggcgtgcgggtgttcgagcgggccgaggtgcccaagaccggcgactccctggccatggcccggcggctggcccggtccgtgcggcggctgacccggcggcgggcccaccggctgctgcgggcccggcggctgctgaagcgggagggcgtgctgcaggccgccgacttcgacgagaacggcctgatcaagtccctgcccaacaccccctggcagctgcgggccgccgccctggaccggaagctgacccccctggagtggtccgccgtgctgctgcacctgatcaagcaccggggctacctgtcccagcggaagaacgagggcgagaccgccgacaaggagctgggcgccctgctgaagggcgtggccaacaacgcccacgccctgcagaccggcgacttccggacccccgccgagctggccctgaacaagttcgagaaggagtccggccacatccggaaccagcggggcgactactcccacaccttctcccggaaggacctgcaggccgagctgatcctgctgttcgagaagcagaaggagttcggcaacccccacgtgtccggcggcctgaaggagggcatcgagaccctgctgatgacccagcggcccgccctgtccggcgacgccgtgcagaagatgctgggccactgcaccttcgagcccgccgagcccaaggccgccaagaacacctacaccgccgagcggttcatctggctgaccaagctgaacaacctgcggatcctggagcagggctccgagcggcccctgaccgacaccgagcgggccaccctgatggacgagccctaccggaagtccaagctgacctacgcccaggcccggaagctgctgggcctggaggacaccgccttcttcaagggcctgcggtacggcaaggacaacgccgaggcctccaccctgatggagatgaaggcctaccacgccatctcccgggccctggagaaggagggcctgaaggacaagaagtcccccctgaacctgtcctccgagctgcaggacgagatcggcaccgccttctccctgttcaagaccgacgaggacatcaccggccggctgaaggaccgggtgcagcccgagatcctggaggccctgctgaagcacatctccttcgacaagttcgtgcagatctccctgaaggccctgcggcggatcgtgcccctgatggagcagggcaagcggtacgacgaggcctgcgccgagatctacggcgaccactacggcaagaagaacaccgaggagaagatctacctgccccccatccccgccgacgagatccggaaccccgtggtgctgcgggccctgtcccaggcccggaaggtgatcaacggcgtggtgcggcggtacggctcccccgcccggatccacatcgagaccgcccgggaggtgggcaagtccttcaaggaccggaaggagatcgagaagcggcaggaggagaaccggaaggaccgggagaaggccgccgccaagttccgggagtacttccccaacttcgtgggcgagcccaagtccaaggacatcctgaagctgcggctgtacgagcagcagcacggcaagtgcctgtactccggcaaggagatcaacctggtgcggctgaacgagaagggctacgtggagatcgaccacgccctgcccttctcccggacctgggacgactccttcaacaacaaggtgctggtgctgggctccgagaaccagaacaagggcaaccagaccccctacgagtacttcaacggcaaggacaactcccgggagtggcaggagttcaaggcccgggtggagacctcccggttcccccggtccaagaagcagcggatcctgctgcagaagttcgacgaggacggcttcaaggagtgcaacctgaacgacacccggtacgtgaaccggttcctgtgccagttcgtggccgaccacatcctgctgaccggcaagggcaagcggcgggtgttcgcctccaacggccagatcaccaacctgctgcggggcttctggggcctgcggaaggtgcgggccgagaacgaccggcaccacgccctggacgccgtggtggtggcctgctccaccgtggccatgcagcagaagatcacccggttcgtgcggtacaaggagatgaacgccttcgacggcaagaccatcgacaaggagaccggcaaggtgctgcaccagaagacccacttcccccagccctgggagttcttcgcccaggaggtgatgatccgggtgttcggcaagcccgacggcaagcccgagttcgaggaggccgacacccccgagaagctgcggaccctgctggccgagaagctgtcctcccggcccgaggccgtgcacgagtacgtgacccccctgttcgtgtcccgggcccccaaccggaagatgtccggcgcccacaaggacaccctgcggtccgccaagcggttcgtgaagcacaacgagaagatctccgtgaagcgggtgtggctgaccgagatcaagctggccgacctggagaacatggtgaactacaagaacggccgggagatcgagctgtacgaggccctgaaggcccggctggaggcctacggcggcaacgccaagcaggccttcgaccccaaggacaaccccttctacaagaagggcggccagctggtgaaggccgtgcgggtggagaagacccaggagtccggcgtgctgctgaacaagaagaacgcctacaccatcgccgacaacggcgacatggtgcgggtggacgtgttctgcaaggtggacaagaagggcaagaaccagtacttcatcgtgcccatctacgcctggcaggtggccgagaacatcctgcccgacatcgactgcaagggctaccggatcgacgactcctacaccttctgcttctccctgcacaagtacgacctgatcgccttccagaaggacgagaagtccaaggtggagttcgcctactacatcaactgcgactcctccaacggccggttctacctggcctggcacgacaagggctccaaggagcagcagttccggatctccacccagaacctggtgctgatccagaagtaccaggtgaacgagctgggcaaggagatccggccctgccggctgaagaagcggccccccgtgcggtag

SEQ ID NO: 379 - DNA, 3420 bp, synthetic construct
  misc_feature 1..3420: note = Synthetic
  source 1..3420: mol_type = other DNA
atggacggctccggcggcggctcccccaagaagaagcggaaggtggaggacaagcggcccgccgccaccaagaaggccggccaggccaagaagaagaagggcggctccggcggcggcgccgccttcaagcccaaccccatcaactacatcctgggcctggacatcggcatcgcctccgtgggctgggccatggtggagatcgacgaggaggagaaccccatccggctgatcgacctgggcgtgcgggtgttcgagcgggccgaggtgcccaagaccggcgactccctggccatggcccggcggctggcccggtccgtgcggcggctgacccggcggcgggcccaccggctgctgcgggcccggcggctgctgaagcgggagggcgtgctgcaggccgccgacttcgacgagaacggcctgatcaagtccctgcccaacaccccctggcagctgcgggccgccgccctggaccggaagctgacccccctggagtggtccgccgtgctgctgcacctgatcaagcaccggggctacctgtcccagcggaagaacgagggcgagaccgccgacaaggagctgggcgccctgctgaagggcgtggccaacaacgcccacgccctgcagaccggcgacttccggacccccgccgagctggccctgaacaagttcgagaaggagtccggccacatccggaaccagcggggcgactactcccacaccttctcccggaaggacctgcaggccgagctgatcctgctgttcgagaagcagaaggagttcggcaacccccacgtgtccggcggcctgaaggagggcatcgagaccctgctgatgacccagcggcccgccctgtccggcgacgccgtgcagaagatgctgggccactgcaccttcgagcccgccgagcccaaggccgccaagaacacctacaccgccgagcggttcatctggctgaccaagctgaacaacctgcggatcctggagcagggctccgagcggcccctgaccgacaccgagcgggccaccctgatggacgagccctaccggaagtccaagctgacctacgcccaggcccggaagctgctgggcctggaggacaccgccttcttcaagggcctgcggtacggcaaggacaacgccgaggcctccaccctgatggagatgaaggcctaccacgccatctcccgggccctggagaaggagggcctgaaggacaagaagtcccccctgaacctgtcctccgagctgcaggacgagatcggcaccgccttctccctgttcaagaccgacgaggacatcaccggccggctgaaggaccgggtgcagcccgagatcctggaggccctgctgaagcacatctccttcgacaagttcgtgcagatctccctgaaggccctgcggcggatcgtgcccctgatggagcagggcaagcggtacgacgaggcctgcgccgagatctacggcgaccactacggcaagaagaacaccgaggagaagatctacctgccccccatccccgccgacgagatccggaaccccgtggtgctgcgggccctgtcccaggcccggaaggtgatcaacggcgtggtgcggcggtacggctcccccgcccggatccacatcgagaccgcccgggaggtgggcaagtccttcaaggaccggaaggagatcgagaagcggcaggaggagaaccggaaggaccgggagaaggccgccgccaagttccgggagtacttccccaacttcgtgggcgagcccaagtccaaggacatcctgaagctgcggctgtacgagcagcagcacggcaagtgcctgtactccggcaaggagatcaacctggtgcggctgaacgagaagggctacgtggagatcgaccacgccctgcccttctcccggacctgggacgactccttcaacaacaaggtgctggtgctgggctccgagaaccagaacaagggcaaccagaccccctacgagtacttcaacggcaaggacaactcccgggagtggcaggagttcaaggcccgggtggagacctcccggttcccccggtccaagaagcagcggatcctgctgcagaagttcgacgaggacggcttcaaggagtgcaacctgaacgacacccggtacgtgaaccggttcctgtgccagttcgtggccgaccacatcctgctgaccggcaagggcaagcggcgggtgttcgcctccaacggccagatcaccaacctgctgcggggcttctggggcctgcggaaggtgcgggccgagaacgaccggcaccacgccctggacgccgtggtggtggcctgctccaccgtggccatgcagcagaagatcacccggttcgtgcggtacaaggagatgaacgccttcgacggcaagaccatcgacaaggagaccggcaaggtgctgcaccagaagacccacttcccccagccctgggagttcttcgcccaggaggtgatgatccgggtgttcggcaagcccgacggcaagcccgagttcgaggaggccgacacccccgagaagctgcggaccctgctggccgagaagctgtcctcccggcccgaggccgtgcacgagtacgtgacccccctgttcgtgtcccgggcccccaaccggaagatgtccggcgcccacaaggacaccctgcggtccgccaagcggttcgtgaagcacaacgagaagatctccgtgaagcgggtgtggctgaccgagatcaagctggccgacctggagaacatggtgaactacaagaacggccgggagatcgagctgtacgaggccctgaaggcccggctggaggcctacggcggcaacgccaagcaggccttcgaccccaaggacaaccccttctacaagaagggcggccagctggtgaaggccgtgcgggtggagaagacccaggagtccggcgtgctgctgaacaagaagaacgcctacaccatcgccgacaacggcgacatggtgcgggtggacgtgttctgcaaggtggacaagaagggcaagaaccagtacttcatcgtgcccatctacgcctggcaggtggccgagaacatcctgcccgacatcgactgcaagggctaccggatcgacgactcctacaccttctgcttctccctgcacaagtacgacctgatcgccttccagaaggacgagaagtccaaggtggagttcgcctactacatcaactgcgactcctccaacggccggttctacctggcctggcacgacaagggctccaaggagcagcagttccggatctccacccagaacctggtgctgatccagaagtaccaggtgaacgagctgggcaaggagatccggccctgccggctgaagaagcggccccccgtgcggtccgagtccgccacccccgagtccgtgtccggctggcggctgttcaagaagatctcctag

SEQ ID NO: 380 - DNA, 3342 bp, synthetic construct
  misc_feature 1..3342: note = Synthetic
  source 1..3342: mol_type = other DNA
atggacggctccggcggcggctccgaggacaagcggcccgccgccaccaagaaggccggccaggccaagaagaagaagggcggctccggcggcggcgccgccttcaagcccaaccccatcaactacatcctgggcctggacatcggcatcgcctccgtgggctgggccatggtggagatcgacgaggaggagaaccccatccggctgatcgacctgggcgtgcgggtgttcgagcgggccgaggtgcccaagaccggcgactccctggccatggcccggcggctggcccggtccgtgcggcggctgacccggcggcgggcccaccggctgctgcgggcccggcggctgctgaagcgggagggcgtgctgcaggccgccgacttcgacgagaacggcctgatcaagtccctgcccaacaccccctggcagctgcgggccgccgccctggaccggaagctgacccccctggagtggtccgccgtgctgctgcacctgatcaagcaccggggctacctgtcccagcggaagaacgagggcgagaccgccgacaaggagctgggcgccctgctgaagggcgtggccaacaacgcccacgccctgcagaccggcgacttccggacccccgccgagctggccctgaacaagttcgagaaggagtccggccacatccggaaccagcggggcgactactcccacaccttctcccggaaggacctgcaggccgagctgatcctgctgttcgagaagcagaaggagttcggcaacccccacgtgtccggcggcctgaaggagggcatcgagaccctgctgatgacccagcggcccgccctgtccggcgacgccgtgcagaagatgctgggccactgcaccttcgagcccgccgagcccaaggccgccaagaacacctacaccgccgagcggttcatctggctgaccaagctgaacaacctgcggatcctggagcagggctccgagcggcccctgaccgacaccgagcgggccaccctgatggacgagccctaccggaagtccaagctgacctacgcccaggcccggaagctgctgggcctggaggacaccgccttcttcaagggcctgcggtacggcaaggacaacgccgaggcctccaccctgatggagatgaaggcctaccacgccatctcccgggccctggagaaggagggcctgaaggacaagaagtcccccctgaacctgtcctccgagctgcaggacgagatcggcaccgccttctccctgttcaagaccgacgaggacatcaccggccggctgaaggaccgggtgcagcccgagatcctggaggccctgctgaagcacatctccttcgacaagttcgtgcagatctccctgaaggccctgcggcggatcgtgcccctgatggagcagggcaagcggtacgacgaggcctgcgccgagatctacggcgaccactacggcaagaagaacaccgaggagaagatctacctgccccccatccccgccgacgagatccggaaccccgtggtgctgcgggccctgtcccaggcccggaaggtgatcaacggcgtggtgcggcggtacggctcccccgcccggatccacatcgagaccgcccgggaggtgggcaagtccttcaaggaccggaaggagatcgagaagcggcaggaggagaaccggaaggaccgggagaaggccgccgccaagttccgggagtacttccccaacttcgtgggcgagcccaagtccaaggacatcctgaagctgcggctgtacgagcagcagcacggcaagtgcctgtactccggcaaggagatcaacctggtgcggctgaacgagaagggctacgtggagatcgaccacgccctgcccttctcccggacctgggacgactccttcaacaacaaggtgctggtgctgggctccgagaaccagaacaagggcaaccagaccccctacgagtacttcaacggcaaggacaactcccgggagtggcaggagttcaaggcccgggtggagacctcccggttcccccggtccaagaagcagcggatcctgctgcagaagttcgacgaggacggcttcaaggagtgcaacctgaacgacacccggtacgtgaaccggttcctgtgccagttcgtggccgaccacatcctgctgaccggcaagggcaagcggcgggtgttcgcctccaacggccagatcaccaacctgctgcggggcttctggggcctgcggaaggtgcgggccgagaacgaccggcaccacgccctggacgccgtggtggtggcctgctccaccgtggccatgcagcagaagatcacccggttcgtgcggtacaaggagatgaacgccttcgacggcaagaccatcgacaaggagaccggcaaggtgctgcaccagaagacccacttcccccagccctgggagttcttcgcccaggaggtgatgatccgggtgttcggcaagcccgacggcaagcccgagttcgaggaggccgacacccccgagaagctgcggaccctgctggccgagaagctgtcctcccggcccgaggccgtgcacgagtacgtgacccccctgttcgtgtcccgggcccccaaccggaagatgtccggcgcccacaaggacaccctgcggtccgccaagcggttcgtgaagcacaacgagaagatctccgtgaagcgggtgtggctgaccgagatcaagctggccgacctggagaacatggtgaactacaagaacggccgggagatcgagctgtacgaggccctgaaggcccggctggaggcctacggcggcaacgccaagcaggccttcgaccccaaggacaaccccttctacaagaagggcggccagctggtgaaggccgtgcgggtggagaagacccaggagtccggcgtgctgctgaacaagaagaacgcctacaccatcgccgacaacggcgacatggtgcgggtggacgtgttctgcaaggtggacaagaagggcaagaaccagtacttcatcgtgcccatctacgcctggcaggtggccgagaacatcctgcccgacatcgactgcaagggctaccggatcgacgactcctacaccttctgcttctccctgcacaagtacgacctgatcgccttccagaaggacgagaagtccaaggtggagttcgcctactacatcaactgcgactcctccaacggccggttctacctggcctggcacgacaagggctccaaggagcagcagttccggatctccacccagaacctggtgctgatccagaagtaccaggtgaacgagctgggcaaggagatccggccctgccggctgaagaagcggccccccgtgcggtag

SEQ ID NO: 381 - DNA, 4005 bp, synthetic construct
  misc_feature 1..4005: note = Synthetic
  source 1..4005: mol_type = other DNA
atggacggctccggcggcggctcccccaagaagaagcggaaggtggaggacaagcggcccgccgccaccaagaaggccggccaggccaagaagaagaagggcggctccggcggcggcgaggcctcccccgcctccggcccccggcacctgatggacccccacatcttcacctccaacttcaacaacggcatcggccggcacaagacctacctgtgctacgaggtggagcggctggacaacggcacctccgtgaagatggaccagcaccggggcttcctgcacaaccaggccaagaacctgctgtgcggcttctacggccggcacgccgagctgcggttcctggacctggtgccctccctgcagctggaccccgcccagatctaccgggtgacctggttcatctcctggtccccctgcttctcctggggctgcgccggcgaggtgcgggccttcctgcaggagaacacccacgtgcggctgcggatcttcgccgcccggatctacgactacgaccccctgtacaaggaggccctgcagatgctgcgggacgccggcgcccaggtgtccatcatgacctacgacgagttcaagcactgctgggacaccttcgtggaccaccagggctgccccttccagccctgggacggcctggacgagcactcccaggccctgtccggccggctgcgggccatcctgcagaaccagggcaactccggctccgagacccccggcacctccgagtccgccacccccgagtccgcagcgttcaaaccaaatcccatcaactacatcctgggcctggccatcggcatcgcctccgtgggctgggccatggtggagatcgacgaggaggagaaccccatccggctgatcgacctgggcgtgcgggtgttcgagcgggccgaggtgcccaagaccggcgactccctggccatggcccggcggctggcccggtccgtgcggcggctgacccggcggcgggcccaccggctgctgcgggcccggcggctgctgaagcgggagggcgtgctgcaggccgccgacttcgacgagaacggcctgatcaagtccctgcccaacaccccctggcagctgcgggccgccgccctggaccggaagctgacccccctggagtggtccgccgtgctgctgcacctgatcaagcaccggggctacctgtcccagcggaagaacgagggcgagaccgccgacaaggagctgggcgccctgctgaagggcgtggccaacaacgcccacgccctgcagaccggcgacttccggacccccgccgagctggccctgaacaagttcgagaaggagtccggccacatccggaaccagcggggcgactactcccacaccttctcccggaaggacctgcaggccgagctgatcctgctgttcgagaagcagaaggagttcggcaacccccacgtgtccggcggcctgaaggagggcatcgagaccctgctgatgacccagcggcccgccctgtccggcgacgccgtgcagaagatgctgggccactgcaccttcgagcccgccgagcccaaggccgccaagaacacctacaccgccgagcggttcatctggctgaccaagctgaacaacctgcggatcctggagcagggctccgagcggcccctgaccgacaccgagcgggccaccctgatggacgagccctaccggaagtccaagctgacctacgcccaggcccggaagctgctgggcctggaggacaccgccttcttcaagggcctgcggtacggcaaggacaacgccgaggcctccaccctgatggagatgaaggcctaccacgccatctcccgggccctggagaaggagggcctgaaggacaagaagtcccccctgaacctgtcctccgagctgcaggacgagatcggcaccgccttctccctgttcaagaccgacgaggacatcaccggccggctgaaggaccgggtgcagcccgagatcctggaggccctgctgaagcacatctccttcgacaagttcgtgcagatctccctgaaggccctgcggcggatcgtgcccctgatggagcagggcaagcggtacgacgaggcctgcgccgagatctacggcgaccactacggcaagaagaacaccgaggagaagatctacctgccccccatccccgccgacgagatccggaaccccgtggtgctgcgggccctgtcccaggcccggaaggtgatcaacggcgtggtgcggcggtacggctcccccgcccggatccacatcgagaccgcccgggaggtgggcaagtccttcaaggaccggaaggagatcgagaagcggcaggaggagaaccggaaggaccgggagaaggccgccgccaagttccgggagtacttccccaacttcgtgggcgagcccaagtccaaggacatcctgaagctgcggctgtacgagcagcagcacggcaagtgcctgtactccggcaaggagatcaacctggtgcggctgaacgagaagggctacgtggagatcgaccacgccctgcccttctcccggacctgggacgactccttcaacaacaaggtgctggtgctgggctccgagaaccagaacaagggcaaccagaccccctacgagtacttcaacggcaaggacaactcccgggagtggcaggagttcaaggcccgggtggagacctcccggttcccccggtccaagaagcagcggatcctgctgcagaagttcgacgaggacggcttcaaggagtgcaacctgaacgacacccggtacgtgaaccgcttcctgtgccagttcgtggccgaccacatcctgctgaccggcaagggcaagcggcgggtgttcgcctccaacggccagatcaccaacctgctgcggggcttctggggcctgcggaaggtgcgggccgagaacgaccggcaccacgccctggacgccgtggtggtggcctgctccaccgtggccatgcagcagaagatcacccggttcgtgcggtacaaggagatgaacgccttcgacggcaagaccatcgacaaggagaccggcaaggtgctgcaccagaagacccacttcccccagccctgggagttcttcgcccaggaggtgatgatccgggtgttcggcaagcccgacggcaagcccgagttcgaggaggccgacacccccgagaagctgcggaccctgctggccgagaagctgtcctcccggcccgaggccgtgcacgagtacgtgacccccctgttcgtgtcccgggcccccaaccggaagatgtccggcgcccacaaggacaccctgcggtccgccaagcggttcgtgaagcacaacgagaagatctccgtgaagcgggtgtggctgaccgagatcaagctggccgacctggagaacatggtgaactacaagaacggccgggagatcgagctgtacgaggccctgaaggcccggctggaggcctacggcggcaacgccaagcaggccttcgaccccaaggacaaccccttctacaagaagggcggccagctggtgaaggccgtgcgggtggagaagacccaggagtccggcgtgctgctgaacaagaagaacgcctacaccatcgccgacaacggcgacatggtgcgggtggacgtgttctgcaaggtggacaagaagggcaagaaccagtacttcatcgtgcccatctacgcctggcaggtggccgagaacatcctgcccgacatcgactgcaagggctaccggatcgacgactcctacaccttctgcttctccctgcacaagtacgacctgatcgccttccagaaggacgagaagtccaaggtggagttcgcctactacatcaactgcgactcctccaacggccggttctacctggcctggcacgacaagggctccaaggagcagcagttccggatctccacccagaacctggtgctgatccagaagtaccaggtgaacgagctgggcaaggagatccggccctgccggctgaagaagcggccccccgtgcggtag

SEQ ID NO: 382 - DNA, 4782 bp, synthetic construct
  misc_feature 1..4782: note = Synthetic
  source 1..4782: mol_type = other DNA
atggaggcctcccccgcctccggcccccggcacctgatggacccccacatcttcacctccaacttcaacaacggcatcggccggcacaagacctacctgtgctacgaggtggagcggctggacaacggcacctccgtgaagatggaccagcaccggggcttcctgcacaaccaggccaagaacctgctgtgcggcttctacggccggcacgccgagctgcggttcctggacctggtgccctccctgcagctggaccccgcccagatctaccgggtgacctggttcatctcctggtccccctgcttctcctggggctgcgccggcgaggtgcgggccttcctgcaggagaacacccacgtgcggctgcggatcttcgccgcccggatctacgactacgaccccctgtacaaggaggccctgcagatgctgcgggacgccggcgcccaggtgtccatcatgacctacgacgagttcaagcactgctgggacaccttcgtggaccaccagggctgccccttccagccctgggacggcctggacgagcactcccaggccctgtccggccggctgcgggccatcctgcagaaccagggcaactccggctccgagacccccggcacctccgagtccgccacccccgagtccgacaagaagtactccatcggcctggccatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtga

SEQ ID NO: 383-399 - skipped (17 entries)

SEQ ID NO: 400 - RNA, 76 nt, synthetic construct
  misc_feature 1..76: note = Synthetic: Conserved Portion of a spyCas9 sgRNA
  source 1..76: mol_type = other RNA
gttttagagctagaaatagcaagttaaaataaggctagtccgttatcaacttgaaaaagtggcaccgagtcggtgc

SEQ ID NO: 401-499 - skipped (99 entries)

SEQ ID NO: 500 - RNA, 145 nt, synthetic construct
  misc_feature 1..145: note = Synthetic
  source 1..145: mol_type = other RNA
nnnnnnnnnnnnnnnnnnnnnnnngttgtagctccctttctcatttcggaaacgaaatgagaaccgttgctacaataaggccgtctgaaaagatgtgccgcaacgctctgccccttaaagcttctgctttaaggggcatcgttta

SEQ ID NO: 501 - RNA, 105 nt, synthetic construct
  misc_feature 1..105: note = Synthetic
  source 1..105: mol_type = other RNA
nnnnnnnnnnnnnnnnnnnnnnnngttgtagctcccttcgaaagaccgttgctacaataaggccgtcgaaagatgtgccgcaacgctctgccttctggcatcgtt

SEQ ID NO: 502-609 - skipped (108 entries)

SEQ ID NO: 610 - RNA, 101 nt, synthetic construct
  misc_feature 1..101: note = Synthetic
  source 1..101: mol_type = other RNA
ccaagtgtcttccagtacgatttggttgtagctccctgaaaccgttgctacaataaggccgtcgaaagatgtgccgcaacgctctgccttctggcatcgtt

SEQ ID NO: 611 - RNA, 93 nt, synthetic construct
  misc_feature 1..93: note = Synthetic
  source 1..93: mol_type = other RNA
ccaagtgtcttccagtacgatttggttgtagctcccttcgaccgttgctacaataaggccgtcgatgtgccgcaacgctctgccggcatcgtt

SEQ ID NO: 612 - RNA, 93 nt, synthetic construct; identical to SEQ ID NO: 611
  misc_feature 1..93: note = Synthetic
  source 1..93: mol_type = other RNA

SEQ ID NO: 613 - RNA, 101 nt, synthetic construct
  misc_feature 1..101: note = Synthetic
  source 1..101: mol_type = other RNA
cttcaccaggagaagccgtcacacgttgtagctccctgaaaccgttgctacaataaggccgtcgaaagatgtgccgcaacgctctgccttctggcatcgtt

SEQ ID NO: 614 - RNA, 93 nt, synthetic construct
  misc_feature 1..93: note = Synthetic
  source 1..93: mol_type = other RNA
cttcaccaggagaagccgtcacacgttgtagctcccttcgaccgttgctacaataaggccgtcgatgtgccgcaacgctctgccggcatcgtt

SEQ ID NO: 615 - RNA, 89 nt, synthetic construct
  misc_feature 1..89: note = Synthetic
  source 1..89: mol_type = other RNA
ccaagtgtcttccagtacgatttggttgtagctccctccgttgctacaataaggccgtcgatgtgccgcaacgctctgccggcatcgtt

SEQ ID NO: 616 - RNA, 89 nt, synthetic construct
  misc_feature 1..89: note = Synthetic
  source 1..89: mol_type = other RNA
cttcaccaggagaagccgtcacacgttgtagctccctccgttgctacaataaggccgtcgatgtgccgcaacgctctgccggcatcgtt

SEQ ID NO: 617 - RNA, 89 nt, synthetic construct; identical to SEQ ID NO: 616
  misc_feature 1..89: note = Synthetic
  source 1..89: mol_type = other RNA

SEQ ID NO: 618 - RNA, 89 nt, synthetic construct; identical to SEQ ID NO: 616
  misc_feature 1..89: note = Synthetic
  source 1..89: mol_type = other RNA

SEQ ID NO: 619 - RNA, 89 nt, synthetic construct; identical to SEQ ID NO: 616
  misc_feature 1..89: note = Synthetic
  source 1..89: mol_type = other RNA

SEQ ID NO: 620 - RNA, 89 nt, synthetic construct; identical to SEQ ID NO: 616
  misc_feature 1..89: note = Synthetic
  source 1..89: mol_type = other RNA

SEQ ID NO: 621 - RNA, 89 nt, synthetic construct; identical to SEQ ID NO: 616
  misc_feature 1..89: note = Synthetic
  source 1..89: mol_type = other RNA

SEQ ID NO: 622 - RNA, 89 nt, synthetic construct; identical to SEQ ID NO: 616
  misc_feature 1..89: note = Synthetic
  source 1..89: mol_type = other RNA

SEQ ID NO: 623 - RNA, 89 nt, synthetic construct; identical to SEQ ID NO: 616
  misc_feature 1..89: note = Synthetic
  source 1..89: mol_type = other RNA

SEQ ID NO: 624 - RNA, 89 nt, synthetic construct; identical to SEQ ID NO: 616
  misc_feature 1..89: note = Synthetic
  source 1..89: mol_type = other RNA

SEQ ID NO: 625 - RNA, 89 nt, synthetic construct; identical to SEQ ID NO: 616
  misc_feature 1..89: note = Synthetic
  source 1..89: mol_type = other RNA

SEQ ID NO: 626 - RNA, 89 nt, synthetic construct; identical to SEQ ID NO: 616
  misc_feature 1..89: note = Synthetic
  source 1..89: mol_type = other RNA

SEQ ID NO: 627 - RNA, 89 nt, synthetic construct; identical to SEQ ID NO: 616
  misc_feature 1..89: note = Synthetic
  source 1..89: mol_type = other RNA

SEQ ID NO: 628 - RNA, 89 nt, synthetic construct; identical to SEQ ID NO: 616
  misc_feature 1..89: note = Synthetic
  source 1..89: mol_type = other RNA

SEQ ID NO: 629 - RNA, 89 nt, synthetic construct; identical to SEQ ID NO: 616
  misc_feature 1..89: note = Synthetic
  source 1..89: mol_type = other RNA

SEQ ID NO: 630 - RNA, 89 nt, synthetic construct; identical to SEQ ID NO: 616
  misc_feature 1..89: note = Synthetic
  source 1..89: mol_type = other RNA

SEQ ID NO: 631 - RNA, 89 nt, synthetic construct; identical to SEQ ID NO: 616
  misc_feature 1..89: note = Synthetic
  source 1..89: mol_type = other RNA

SEQ ID NO: 632 - RNA, 89 nt, synthetic construct; identical to SEQ ID NO: 616
  misc_feature 1..89: note = Synthetic
  source 1..89: mol_type = other RNA

SEQ ID NO: 633 - RNA, 89 nt, synthetic construct; identical to SEQ ID NO: 616
  misc_feature 1..89: note = Synthetic
  source 1..89: mol_type = other RNA

SEQ ID NO: 634 - RNA, 89 nt, synthetic construct; identical to SEQ ID NO: 615
  misc_feature 1..89: note = Synthetic
  source 1..89: mol_type = other RNA

SEQ ID NO: 635 - RNA, 89 nt, synthetic construct; identical to SEQ ID NO: 615
  misc_feature 1..89: note = Synthetic
  source 1..89: mol_type = other RNA

SEQ ID NO: 636 - RNA, 89 nt, synthetic construct; identical to SEQ ID NO: 615
  misc_feature 1..89: note = Synthetic
  source 1..89: mol_type = other RNA

SEQ ID NO: 637 - RNA, 89 nt, synthetic construct; identical to SEQ ID NO: 615
  misc_feature 1..89: note = Synthetic
  source 1..89: mol_type = other RNA

SEQ ID NO: 638 - RNA, 89 nt, synthetic construct; identical to SEQ ID NO: 615
  misc_feature 1..89: note = Synthetic
  source 1..89: mol_type = other RNA

SEQ ID NO: 639 - RNA, 89 nt, synthetic construct; identical to SEQ ID NO: 615
  misc_feature 1..89: note = Synthetic
  source 1..89: mol_type = other RNA

SEQ ID NO: 640 - RNA, 89 nt, synthetic construct; identical to SEQ ID NO: 615
  misc_feature 1..89: note = Synthetic
  source 1..89: mol_type = other RNA

SEQ ID NO: 641 - RNA, 89 nt, synthetic construct; identical to SEQ ID NO: 615
  misc_feature 1..89: note = Synthetic
  source 1..89: mol_type = other RNA

SEQ ID NO: 642 - RNA, 89 nt, synthetic construct; identical to SEQ ID NO: 615
  misc_feature 1..89: note = Synthetic
  source 1..89: mol_type = other RNA

SEQ ID NO: 643 - RNA, 89 nt, synthetic construct; identical to SEQ ID NO: 615
  misc_feature 1..89: note = Synthetic
  source 1..89: mol_type = other RNA

SEQ ID NO: 644 - RNA, 89 nt, synthetic construct; identical to SEQ ID NO: 615
  misc_feature 1..89: note = Synthetic
  source 1..89: mol_type = other RNA

SEQ ID NO: 645 - RNA, 89 nt, synthetic construct; identical to SEQ ID NO: 615
  misc_feature 1..89: note = Synthetic
  source 1..89: mol_type = other RNA

SEQ ID NO: 646 - RNA, 89 nt, synthetic construct; identical to SEQ ID NO: 615
  misc_feature 1..89: note = Synthetic
  source 1..89: mol_type = other RNA

SEQ ID NO: 647 - RNA, 89 nt, synthetic construct; identical to SEQ ID NO: 615
  misc_feature 1..89: note = Synthetic
  source 1..89: mol_type = other RNA

SEQ ID NO: 648 - RNA, 89 nt, synthetic construct; identical to SEQ ID NO: 615
  misc_feature 1..89: note = Synthetic
  source 1..89: mol_type = other RNA

SEQ ID NO: 649 - RNA, 89 nt, synthetic construct; identical to SEQ ID NO: 615
  misc_feature 1..89: note = Synthetic
  source 1..89: mol_type = other RNA

SEQ ID NO: 650 - RNA, 89 nt, synthetic construct; identical to SEQ ID NO: 615
  misc_feature 1..89: note = Synthetic
  source 1..89: mol_type = other RNA

SEQ ID NO: 651 - RNA, 89 nt, synthetic construct
  misc_feature 1..89: note = Synthetic
  source 1..89: mol_type = other RNA
cttcaccaagagaagccgtcacacgttgtagctccctccgttgctacaataaggccgtcgatgtgccgcaacgctctgccggcatcgtt

SEQ ID NO: 652 - RNA, 89 nt, synthetic construct; identical to SEQ ID NO: 615
  misc_feature 1..89: note = Synthetic
  source 1..89: mol_type = other RNA

SEQ ID NO: 653 - RNA, 93 nt, synthetic construct
  misc_feature 1..93: note = Synthetic
  source 1..93: mol_type = other RNA
ccaagtgtcttccagtacgatttggttgtagctcccttgcaccgttgctacaataaggccgtcgatgtgccgcaacgctctgccggcatcgtt

SEQ ID NO: 654 - RNA, 91 nt, synthetic construct
  misc_feature 1..91: note = Synthetic
  source 1..91: mol_type = other RNA
ccaagtgtcttccagtacgatttggttgtagctccctgcccgttgctacaataaggccgtcgatgtgccgcaacgctctgccggcatcgtt

SEQ ID NO: 655 - RNA, 89 nt, synthetic construct
  misc_feature 1..89: note = Synthetic
  source 1..89: mol_type = other RNA
ccaagtgtcttccagtacgatttggttgtagctcccgccgttgctacaataaggccgtcgatgtgccgcaacgctctgccggcatcgtt

SEQ ID NO: 656 - RNA, 87 nt, synthetic construct
  misc_feature 1..87: note = Synthetic
  source 1..87: mol_type = other RNA
ccaagtgtcttccagtacgatttggttgtagctccgcgttgctacaataaggccgtcgatgtgccgcaacgctctgccggcatcgtt

SEQ ID NO: 657 - RNA, 85 nt, synthetic construct
  misc_feature 1..85: note = Synthetic
  source 1..85: mol_type = other RNA
ccaagtgtcttccagtacgatttggttgtagctccgttgctacaataaggccgtcgatgtgccgcaacgctctgccggcatcgtt

SEQ ID NO: 658 - RNA, 95 nt, synthetic construct
  misc_feature 1..95: note = Synthetic
  source 1..95: mol_type = other RNA
ccaagtgtcttccagtacgatttggttgtagctcccttgcaccgttgctacaataaggccgtctagatgtgccgcaacgctctgccggcatcgtt

SEQ ID NO: 659 - RNA, 95 nt, synthetic construct
  misc_feature 1..95: note = Synthetic
  source 1..95: mol_type = other RNA
ccaagtgtcttccagtacgatttggttgtagctcccttgcaccgttgctacaataaggccgtcgatgtgccgcaacgctctgcctaggcatcgtt

SEQ ID NO: 660-709 - skipped (50 entries)

SEQ ID NO: 710 - RNA, 101 nt, synthetic construct; identical to SEQ ID NO: 610
  misc_feature 1..101: note = Synthetic
  variation 1..101: note = 2'-O-Me and PS modifications are present or absent
  source 1..101: mol_type = other RNA

SEQ ID NO: 711 - RNA, 93 nt, synthetic construct; identical to SEQ ID NO: 611
  misc_feature 1..93: note = Synthetic
  variation 1..93: note = internal linker L1, 2'-O-Me and PS linkage modifications are present or absent
  source 1..93: mol_type = other RNA

SEQ ID NO: 712 - RNA, 93 nt, synthetic construct; identical to SEQ ID NO: 611
  misc_feature 1..93: note = Synthetic
  variation 1..93: note = internal linker L1, 2'-O-Me and PS linkage modifications are present or absent
  source 1..93: mol_type = other RNA

SEQ ID NO: 713 - RNA, 101 nt, synthetic construct; identical to SEQ ID NO: 613
  misc_feature 1..101: note = Synthetic
  variation 1..101: note = 2'-O-Me and PS modifications are present or absent
  source 1..101: mol_type = other RNA

SEQ ID NO: 714 - RNA, 93 nt, synthetic construct; identical to SEQ ID NO: 614
  misc_feature 1..93: note = Synthetic
  variation 1..93: note = internal linker L1, 2'-O-Me and PS linkage modifications are present or absent
  source 1..93: mol_type = other RNA

SEQ ID NO: 715 - RNA, 89 nt, synthetic construct; identical to SEQ ID NO: 615
  misc_feature 1..89: note = Synthetic
  variation 1..89: note = internal linker L1, 2'-O-Me and PS linkage modifications are present or absent
  source 1..89: mol_type = other RNA

SEQ ID NO: 716 - RNA, 89 nt, synthetic construct; identical to SEQ ID NO: 616
  misc_feature 1..89: note = Synthetic
  variation 1..89: note = internal linker L1, 2'-O-Me and PS linkage modifications are present or absent
  source 1..89: mol_type = other RNA

SEQ ID NO: 717 - RNA, 89 nt, synthetic construct; identical to SEQ ID NO: 616
  misc_feature 1..89: note = Synthetic
  variation 1..89: note = internal linker L1, 2'-O-Me and PS linkage modifications are present or absent
  source 1..89: mol_type = other RNA

SEQ ID NO: 718 - RNA, 89 nt, synthetic construct; identical to SEQ ID NO: 616
  misc_feature 1..89: note = Synthetic
  variation 1..89: note = internal linker L1, 2'-O-Me and PS linkage modifications are present or absent
  source 1..89: mol_type = other RNA

SEQ ID NO: 719 - RNA, 89 nt, synthetic construct; identical to SEQ ID NO: 616
  misc_feature 1..89: note = Synthetic
  variation 1..89: note = internal linker L1, 2'-O-Me and PS linkage modifications are present or absent
  source 1..89: mol_type = other RNA

SEQ ID NO: 720 - RNA, 89 nt, synthetic construct; identical to SEQ ID NO: 616
  misc_feature 1..89: note = Synthetic
  variation 1..89: note = internal linker L1, 2'-O-Me and PS linkage modifications are present or absent
  source 1..89: mol_type = other RNA

SEQ ID NO: 721 - RNA, 89 nt, synthetic construct; identical to SEQ ID NO: 616
  misc_feature 1..89: note = Synthetic
  variation 1..89: note = internal linker L1, 2'-O-Me and PS linkage modifications are present or absent
  source 1..89: mol_type = other RNA

SEQ ID NO: 722 - RNA, 89 nt, synthetic construct; identical to SEQ ID NO: 616
  misc_feature 1..89: note = Synthetic
  variation 1..89: note = internal linker L1, 2'-O-Me and PS linkage modifications are present or absent
  source 1..89: mol_type = other RNA

SEQ ID NO: 723 - RNA, 89 nt, synthetic construct; identical to SEQ ID NO: 616
  misc_feature 1..89: note = Synthetic
  variation 1..89: note = internal linker L1, 2'-O-Me and PS linkage modifications are present or absent
  source 1..89: mol_type = other RNA

SEQ ID NO: 724 - RNA, 89 nt, synthetic construct; identical to SEQ ID NO: 616
  misc_feature 1..89: note = Synthetic
  variation 1..89: note = internal linker L1, 2'-O-Me and PS linkage modifications are present or absent
  source 1..89: mol_type = other RNA

SEQ ID NO: 725 - RNA, 89 nt, synthetic construct; identical to SEQ ID NO: 616
  misc_feature 1..89: note = Synthetic
  variation 1..89: note = internal linker L1, 2'-O-Me and PS linkage modifications are present or absent
  source 1..89: mol_type = other RNA

SEQ ID NO: 726 - RNA, 89 nt, synthetic construct; identical to SEQ ID NO: 616
  misc_feature 1..89: note = Synthetic
  variation 1..89: note = internal linker L1, 2'-O-Me and PS linkage modifications are present or absent
  source 1..89: mol_type = other RNA

SEQ ID NO: 727 - RNA, 89 nt, synthetic construct; identical to SEQ ID NO: 616
  misc_feature 1..89: note = Synthetic
  variation 1..89: note = internal linker L1, 2'-O-Me and PS linkage modifications are present or absent
  source 1..89: mol_type = other RNA

SEQ ID NO: 728 - RNA, 89 nt, synthetic construct; identical to SEQ ID NO: 616
  misc_feature 1..89: note = Synthetic
  variation 1..89: note = internal linker L1, 2'-O-Me and PS linkage modifications are present or absent
  source 1..89: mol_type = other RNA

SEQ ID NO: 729 - RNA, 89 nt, synthetic construct; identical to SEQ ID NO: 616
  misc_feature 1..89: note = Synthetic
  variation 1..89: note = internal linker L1, 2'-O-Me and PS linkage modifications are present or absent
  source 1..89: mol_type = other RNA

SEQ ID NO: 730 - RNA, 89 nt, synthetic construct; identical to SEQ ID NO: 616
  misc_feature 1..89: note = Synthetic
  variation 1..89: note = internal linker L1, 2'-O-Me and PS linkage modifications are present or absent
  source 1..89: mol_type = other RNA

SEQ ID NO: 731 - RNA, 89 nt, synthetic construct; identical to SEQ ID NO: 616
  misc_feature 1..89: note = Synthetic
  variation 1..89: note = internal linker L1, 2'-O-Me and PS linkage modifications are present or absent
  source 1..89: mol_type = other RNA

SEQ ID NO: 732 - RNA, 89 nt, synthetic construct; identical to SEQ ID NO: 616
  misc_feature 1..89: note = Synthetic
  variation 1..89: note = internal linker L1, 2'-O-Me and PS linkage modifications are present or absent
  source 1..89: mol_type = other RNA

SEQ ID NO: 733 - RNA, 89 nt, synthetic construct; identical to SEQ ID NO: 616
  misc_feature 1..89: note = Synthetic
  variation 1..89: note = internal linker L1, 2'-O-Me and PS linkage modifications are present or absent
  source 1..89: mol_type = other RNA

SEQ ID NO: 734 - RNA, 89 nt, synthetic construct; identical to SEQ ID NO: 615
  misc_feature 1..89: note = Synthetic
  variation 1..89: note = internal linker L1, 2'-O-Me and PS linkage modifications are present or absent
  source 1..89: mol_type = other RNA

SEQ ID NO: 735 - RNA, 89 nt, synthetic construct; identical to SEQ ID NO: 615
  misc_feature 1..89: note = Synthetic
  variation 1..89: note = internal linker L1, 2'-O-Me and PS linkage modifications are present or absent
  source 1..89: mol_type = other RNA

SEQ ID NO: 736 - RNA, 89 nt, synthetic construct; identical to SEQ ID NO: 615
  misc_feature 1..89: note = Synthetic
  variation 1..89: note = internal linker L1, 2'-O-Me and PS linkage modifications are present or absent
  source 1..89: mol_type = other RNA

SEQ ID NO: 737 - RNA, 89 nt, synthetic construct; identical to SEQ ID NO: 615
  misc_feature 1..89: note = Synthetic
  variation 1..89: note = internal linker L1, 2'-O-Me and PS linkage modifications are present or absent
  source 1..89: mol_type = other RNA

SEQ ID NO: 738 - RNA, 89 nt, synthetic construct; identical to SEQ ID NO: 615
  misc_feature 1..89: note = Synthetic
  variation 1..89: note = internal linker L1, 2'-O-Me and PS linkage modifications are present or absent
  source 1..89: mol_type = other RNA

SEQ ID NO: 739 - RNA, 89 nt, synthetic construct; identical to SEQ ID NO: 615
  misc_feature 1..89: note = Synthetic
  variation 1..89: note = internal linker L1, 2'-O-Me and PS linkage modifications are present or absent
  source 1..89: mol_type = other RNA

SEQ ID NO: 740 - RNA, 89 nt, synthetic construct; identical to SEQ ID NO: 615
  misc_feature 1..89: note = Synthetic
  variation 1..89: note = internal linker L1, 2'-O-Me and PS linkage modifications are present or absent
  source 1..89: mol_type = other RNA

SEQ ID NO: 741 - RNA, 89 nt, synthetic construct; identical to SEQ ID NO: 615
  misc_feature 1..89: note = Synthetic
  variation 1..89: note = internal linker L1, 2'-O-Me and PS linkage modifications are present or absent
  source 1..89: mol_type = other RNA

SEQ ID NO: 742 - RNA, 89 nt, synthetic construct; identical to SEQ ID NO: 615
  misc_feature 1..89: note = Synthetic
  variation 1..89: note = internal linker L1, 2'-O-Me and PS linkage modifications are present or absent
  source 1..89: mol_type = other RNA

SEQ ID NO: 743 - RNA, 89 nt, synthetic construct; identical to SEQ ID NO: 615
  misc_feature 1..89: note = Synthetic
  variation 1..89: note = internal linker L1, 2'-O-Me and PS linkage modifications are present or absent
  source 1..89: mol_type = other RNA

SEQ ID NO: 744 - RNA, 89 nt, synthetic construct; identical to SEQ ID NO: 615
  misc_feature 1..89: note = Synthetic
  variation 1..89: note = internal linker L1, 2'-O-Me and PS linkage modifications are present or absent
  source 1..89: mol_type = other RNA

SEQ ID NO: 745 - RNA, 89 nt, synthetic construct; identical to SEQ ID NO: 615
  misc_feature 1..89: note = Synthetic
  variation 1..89: note = internal linker L1, 2'-O-Me and PS linkage modifications are present or absent
  source 1..89: mol_type = other RNA

SEQ ID NO: 746 - RNA, 89 nt, synthetic construct; identical to SEQ ID NO: 615
  misc_feature 1..89: note = Synthetic
  variation 1..89: note = internal linker L1, 2'-O-Me and PS linkage modifications are present or absent
  source 1..89: mol_type = other RNA

SEQ ID NO: 747 - RNA, 89 nt, synthetic construct; identical to SEQ ID NO: 615
  misc_feature 1..89: note = Synthetic
  variation 1..89: note = internal linker L1, 2'-O-Me and PS linkage modifications are present or absent
  source 1..89: mol_type = other RNA

SEQ ID NO: 748 - RNA, 89 nt, synthetic construct; identical to SEQ ID NO: 615
  misc_feature 1..89: note = Synthetic
  variation 1..89: note = internal linker L1, 2'-O-Me and PS linkage modifications are present or absent
  source 1..89: mol_type = other RNA

SEQ ID NO: 749 - RNA, 89 nt, synthetic construct; identical to SEQ ID NO: 615
  misc_feature 1..89: note = Synthetic
  variation 1..89: note = internal linker L1, 2'-O-Me and PS linkage modifications are present or absent
  source 1..89: mol_type = other RNA

SEQ ID NO: 750 - RNA, 89 nt, synthetic construct; identical to SEQ ID NO: 615
  misc_feature 1..89: note = Synthetic
  variation 1..89: note = internal linker L1, 2'-O-Me and PS linkage modifications are present or absent
  source 1..89: mol_type = other RNA

SEQ ID NO: 751 - RNA, 89 nt, synthetic construct; identical to SEQ ID NO: 651
  misc_feature 1..89: note = Synthetic
  variation 1..89: note = internal linker L1, 2'-O-Me and PS linkage modifications are present or absent
  source 1..89: mol_type = other RNA

SEQ ID NO: 752 - RNA, 89 nt, synthetic construct; identical to SEQ ID NO: 615
  misc_feature 1..89: note = Synthetic
  variation 1..89: note = internal linker L1, 2'-O-Me and PS linkage modifications are present or absent
  source 1..89: mol_type = other RNA

SEQ ID NO: 753 - RNA, 93 nt, synthetic construct; identical to SEQ ID NO: 653
  misc_feature 1..93: note = Synthetic
  variation 1..93: note = internal linker L1, 2'-O-Me and PS linkage modifications are present or absent
  source 1..93: mol_type = other RNA

SEQ ID NO: 754 - RNA, 91 nt, synthetic construct; identical to SEQ ID NO: 654
  misc_feature 1..91: note = Synthetic
  variation 1..91: note = internal linker L1, 2'-O-Me and PS linkage modifications are present or absent
  source 1..91: mol_type = other RNA

SEQ ID NO: 755 - RNA, 89 nt, synthetic construct; identical to SEQ ID NO: 655
  misc_feature 1..89: note = Synthetic
  variation 1..89: note = internal linker L1, 2'-O-Me and PS linkage modifications are present or absent
  source 1..89: mol_type = other RNA

SEQ ID NO: 756 - RNA, 87 nt, synthetic construct; identical to SEQ ID NO: 656
  misc_feature 1..87: note = Synthetic
  variation 1..87: note = internal linker L1, 2'-O-Me and PS linkage modifications are present or absent
  source 1..87: mol_type = other RNA

SEQ ID NO: 757 - RNA, 85 nt, synthetic construct; identical to SEQ ID NO: 657
  misc_feature 1..85: note = Synthetic
  variation 1..85: note = internal linker L1, 2'-O-Me and PS linkage modifications are present or absent
  source 1..85: mol_type = other RNA

SEQ ID NO: 758 - RNA, 95 nt, synthetic construct; identical to SEQ ID NO: 658
  misc_feature 1..95: note = Synthetic
  variation 1..95: note = internal linker L1, 2'-O-Me and PS linkage modifications are present or absent
  source 1..95: mol_type = other RNA

SEQ ID NO: 759 - RNA, 95 nt, synthetic construct; identical to SEQ ID NO: 659
  misc_feature 1..95: note = Synthetic
  variation 1..95: note = internal linker L1, 2'-O-Me and PS linkage modifications are present or absent
  source 1..95: mol_type = other RNA

SEQ ID NO: 760-999 - skipped (240 entries)

SEQ ID NO: 1000 - RNA, 94 nt, synthetic construct
  misc_feature 1..94: note = Synthetic
  variation 1..94: note = internal linker L1 is present or absent
  variation 21..25: note = n may or may not be present
  source 1..94: mol_type = other RNA
nnnnnnnnnnnnnnnnnnnnnnnnngttgtagctcccttcgaccgttgctacaataaggccgtcgatgtgccgcaacgctctgccggcatcgtt

SEQ ID NO: 1001 - RNA, 93 nt, synthetic construct
  misc_feature 1..93: note = Synthetic
  variation 1..93: note = internal linker L1, 2'-O-Me and PS linkage modifications are present or absent
  source 1..93: mol_type = other RNA
nnnnnnnnnnnnnnnnnnnnnnnngttgtagctcccttcgaccgttgctacaataaggccgtcgatgtgccgcaacgctctgccggcatcgtt

SEQ ID NO: 1002 - RNA, 89 nt, synthetic construct
  misc_feature 1..89: note = Synthetic
  variation 1..89: note = internal linker L1, 2'-O-Me and PS linkage modifications are present or absent
  source 1..89: mol_type = other RNA
nnnnnnnnnnnnnnnnnnnnnnnngttgtagctccctccgttgctacaataaggccgtcgatgtgccgcaacgctctgccggcatcgtt

SEQ ID NO: 1003 - RNA, 89 nt, synthetic construct; identical to SEQ ID NO: 1002
  misc_feature 1..89: note = Synthetic
  variation 1..89: note = internal linker L1, 2'-O-Me and PS linkage modifications are present or absent
  source 1..89: mol_type = other RNA

SEQ ID NO: 1004 - RNA, 90 nt, synthetic construct
  misc_feature 1..90: note = Synthetic
  variation 1..90: note = internal linker L1, 2'-O-Me and PS linkage modifications are present or absent
  variation 21..25: note = n may or may not be present
  source 1..90: mol_type = other RNA
nnnnnnnnnnnnnnnnnnnnnnnnngttgtagctccctccgttgctacaataaggccgtcgatgtgccgcaacgctctgccggcatcgtt

SEQ ID NO: 1005 - RNA, 94 nt, synthetic construct
  misc_feature 1..94: note = Synthetic
  variation 1..94: note = internal linker L1, 2'-O-Me and PS linkage modifications are present or absent
  variation 21..25: note = n may or may not be present
  source 1..94: mol_type = other RNA
nnnnnnnnnnnnnnnnnnnnnnnnngttgtagctcccttgcaccgttgctacaataaggccgtcgatgtgccgcaacgctctgccggcatcgtt

SEQ ID NO: 1006 - RNA, 94 nt, synthetic construct; identical to SEQ ID NO: 1005
  misc_feature 1..94: note = Synthetic
  variation 1..94: note = internal linker L1, 2'-O-Me and PS linkage modifications are present or absent
  variation 21..25: note = n may or may not be present
  source 1..94: mol_type = other RNA

SEQ ID NO: 1007 - RNA, 92 nt, synthetic construct
  misc_feature 1..92: note = Synthetic
  variation 1..92: note = internal linker L1, 2'-O-Me and PS linkage modifications are present or absent
  variation 21..25: note = n may or may not be present
  source 1..92: mol_type = other RNA
nnnnnnnnnnnnnnnnnnnnnnnnngttgtagctccctgcccgttgctacaataaggccgtcgatgtgccgcaacgctctgccggcatcgtt

SEQ ID NO: 1008 - RNA, 90 nt, synthetic construct
  misc_feature 1..90: note = Synthetic
  variation 1..90: note = internal linker L1, 2'-O-Me and PS linkage modifications are present or absent
  variation 21..25: note = n may or may not be present
  source 1..90: mol_type = other RNA
nnnnnnnnnnnnnnnnnnnnnnnnngttgtagctcccgccgttgctacaataaggccgtcgatgtgccgcaacgctctgccggcatcgtt

SEQ ID NO: 1009 - RNA, 88 nt, synthetic construct
  misc_feature 1..88: note = Synthetic
  variation 1..88: note = internal linker L1, 2'-O-Me and PS linkage modifications are present or absent
  variation 21..25: note = n may or may not be present
  source 1..88: mol_type = other RNA
nnnnnnnnnnnnnnnnnnnnnnnnngttgtagctccgcgttgctacaataaggccgtcgatgtgccgcaacgctctgccggcatcgtt

SEQ ID NO: 1010 - RNA, 86 nt, synthetic construct
  misc_feature 1..86: note = Synthetic
  variation 1..86: note = internal linker L1, 2'-O-Me and PS linkage modifications are present or absent
  variation 21..25: note = n may or may not be present
  source 1..86: mol_type = other RNA
nnnnnnnnnnnnnnnnnnnnnnnnngttgtagctccgttgctacaataaggccgtcgatgtgccgcaacgctctgccggcatcgtt

SEQ ID NO: 1011 - RNA, 96 nt, synthetic construct
  misc_feature 1..96: note = Synthetic
  variation 1..96: note = internal linker L1, 2'-O-Me and PS linkage modifications are present or absent
  variation 21..25: note = n may or may not be present
  source 1..96: mol_type = other RNA
nnnnnnnnnnnnnnnnnnnnnnnnngttgtagctcccttgcaccgttgctacaataaggccgtctagatgtgccgcaacgctctgccggcatcgtt

SEQ ID NO: 1012 - RNA, 96 nt, synthetic construct
  misc_feature 1..96: note = Synthetic
  variation 1..96: note = internal linker L1, 2'-O-Me and PS linkage modifications are present or absent
  variation 21..25: note = n may or may not be present
  source 1..96: mol_type = other RNA
nnnnnnnnnnnnnnnnnnnnnnnnngttgtagctcccttgcaccgttgctacaataaggccgtcgatgtgccgcaacgctctgcctaggcatcgtt

(19) United States
(12) Patent Application Publication
(10) Pub. No.: US 2024/0150761 A1
(43) Pub. Date: May 9, 2024

(54) Modified Guide RNAs Comprising an Internal Linker for Gene Editing

(71) Applicant: Intellia Therapeutics, Inc., Cambridge, MA (US)

(72) Inventors: Sabin Mulepati, Somerville, MA (US); Rubina Giare Parmar, Acton, MA (US); Lindsey Jean Stretz, Cambridge, MA (US); Michelle Young, Cambridge, MA (US); Jasmine Josephine Bonanno, Cambridge, MA (US); Seth C. Alexander, Medford, MA (US)

(73) Assignee: Intellia Therapeutics, Inc., Cambridge, MA (US)

(21) Appl. No.: 18/532,127

(22) Filed: Dec. 7, 2023

Related U.S. Application Data
(63) Continuation of application No. PCT/US22/32791, filed on Jun. 9, 2022.
(60) Provisional application No. 63/209,273, filed on Jun. 10, 2021.
(60) Provisional application No. 63/275,427, filed on Nov. 3, 2021.

(51) Int. Cl. C12N 15/113; A61K 48/00; C12N 9/22

(52) U.S. Cl. CPC C12N 15/113; A61K 48/005; C12N 9/22; C12N 2310/315; C12N 2310/321; C12N 2310/322; C12N 2310/533

(57) ABSTRACT

This disclosure relates to modified guide RNAs comprising an internal linker for in vitro and in vivo gene editing methods.

This application is a bypass continuation of International Application No. PCT/US2022/032791, filed on Jun. 9, 2022, which claims the benefit of priority to U.S. Provisional Application No. 63/209,273, filed on Jun. 10, 2021, and U.S. Provisional Application No. 63/275,427, filed on Nov. 3, 2021, the contents of each of which are incorporated by reference in its entirety.

The instant application contains a Sequence Listing which has been submitted electronically in XMLformat and is hereby incorporated by reference in its entirety. Said XML copy, created on Dec. 4, 2023, is named 01155-0047-00US-ST26.XML and is 816,160 bytes in size.

This disclosure relates to the field of gene editing using CRISPR/Cas systems, a part of the prokaryotic immune system that recognizes and cuts exogenous genetic elements.

The prokaryotic CRISPR/Cas system relies on nucleases, having RNA and DNA binding activity, or a DNA-binding subunit of such a complex, wherein the DNA binding activity is sequence-specific and depends on the sequence of the RNA. Such complexes, often referred to as RNA-guided DNA binding agents, include a number of RNA-guided DNA binding agents including Cas cleavases/nickases. Cas cleavases and Cas nickases include a Csm or Cmr complex of a type III CRISPR system, the Cas10, Csm1, or Cmr2 subunit thereof, a Cascade complex of a type I CRISPR system, the Cas3 subunit thereof, and Class 2 Cas nucleases. Exemplary monomeric nucleases, such as Cas9, termed CRISPR-associated protein 9 (Cas9), induce site-specific breaks in DNA. Guide RNAs are commonly prepared by in vitro oligonucleotide synthesis. Given the cyclic nature and imperfect yield of oligonucleotide synthesis, substituting a non-nucleic acid internal linker for portions of the gRNA while retaining or even improving its activity would be desirable, e.g., so that the gRNA can be obtained in greater yield (e.g., due to fewer cycles of nucleotide addition), or compositions comprising the gRNA have greater homogeneity or fewer incomplete or erroneous products. Additionally, improved methods and compositions for preventing such degradation, improving stability of gRNAs and enhancing gene editing efficiency is desired, especially for therapeutic applications.

### SUMMARY

In some embodiments, genome editing tools are provided comprising guide RNA (gRNA) comprising an internal linker as described herein. The present application stems from the findings that a non-nucleic acid linker can replace certain inner portions of the guide RNAs that have non-essential contacts with Cas nuclease. The substitutions described herein may facilitate synthesis of the gRNA with greater yield or homogeneity; or may improve the stability of the gRNA and its corresponding nuclease, e.g., the gRNA/Cas complex and improve the activity of a Cas9 (e.g., SauCas9, SpyCas9, CdiCas9, St1Cas9, SthCas9, AceCas9, CjeCas9, RpaCas9, RruCas9, AnaCas9, NmeCas9), Cas12 (e.g., AsCas12a, LbCpf1), or Cas13 (e.g., EsCas13d) to modify target DNA.

In some embodiments, a single-guide RNA (sgRNA) with one or more substitutions to include one or more internal linkers as described herein are provided.

In some embodiments, crisprRNA (crRNA) or tracrRNA (trRNA) with one or more substitutions to include one or more internal linkers as described herein are provided. In some embodiments, the modified crRNA or modified trRNA comprise a dual guide RNA (dgRNA). In some embodiments, the modified crRNA or modified trRNA comprise a single guide RNA (sgRNA). The substitutions with one or more internal linkers as described herein may facilitate synthesis of the gRNA with greater yield or homogeneity; or may improve the stability of the gRNA and its corresponding nuclease, e.g., the gRNA/Cas complex, e.g., the gRNA/Cas9 complex and improve the activity of the nuclease, e.g., a Cas9 nuclease (e.g., SauCas9, SpyCas9) e.g., to cleave or nick the target DNA. Compared to guides comprised of all nucleotides, e.g., 100mer Spy Cas 9 sgRNAs or other short guide Spy Cas9 RNAs, synthesis of the presently disclosed guide RNAs may increase crude yield of a guide RNA. Similarly, gRNA sample purity as measured by the proportion of full-length product, e.g. crude purity, can be increased. gRNA can be obtained in greater yield, or compositions comprising the gRNA can have greater homogeneity or fewer incomplete or erroneous products. Guide RNA purity may be assessed using ion-pair reversed-phase high performance liquid chromatography (IP-RP-HPLC) and ion exchange HPLC methods, e.g. as in Kanavarioti et al, Sci Rep 9, 1019 (2019) (doi:10.1038/s41598-018-37642-z). Using UV spectroscopy at a wavelength of 260 nm, crude purity and final purity can be determined by the ratio of absorbance of the main peak to the cumulative absorbance of all peaks in the chromatogram. Synthetic yield is determined as the ratio of the absorbance at 260 nm of the final sample compared to the theoretical absorbance of input materials.

The Following Embodiments are Encompassed.

In some embodiments, a guide RNA (gRNA) comprising an internal linker is provided. In some embodiments, the internal linker substitutes for at least 2, 3, 4, 5, 6, 7, 8, 9, 10, 11, 12, 13, 14, 15, 16, 17, 18, 19, 20, 21, 22, 23, 24, 25, 26, 27, or 28 nucleotides of the gRNA. In some embodiments, the internal linker has a bridging length of about 3-30, optionally 12-21 atoms, and the linker substitutes for at least 2 nucleotides of the gRNA. In some embodiments, the internal linker has a bridging length of about 6-18 atoms, optionally about 6-12 atoms, and the linker substitutes for at least 2 nucleotides of the gRNA. In some embodiments, the internal linker substitutes for 2-12 nucleotides.

In some embodiments, the internal linker is in a repeat-anti-repeat region of the gRNA. In some embodiments, the internal linker substitutes for at least 4 nucleotides of the repeat-anti-repeat region of the gRNA. In some embodiments, the internal linker substitutes for up to 28 nucleotides of the repeat-anti-repeat region of the gRNA. In some embodiments, the internal linker substitutes for 2, 3, 4, 5, 6, 7, 8, 9, 10, 11, or 12 nucleotides of the repeat-anti-repeat region of the gRNA.

In some embodiments, the internal linker is in a hairpin region of the gRNA. In some embodiments, the internal linker substitutes for at least 2 nucleotides of the hairpin region of the gRNA. In some embodiments, the internal linker substitutes for up to 22 nucleotides of the hairpin region of the gRNA. In some embodiments, the internal linker substitutes for 1, 2, 3, 4, 5, 6, 7, 8, or 9 base pairs of the hairpin region of the gRNA.

In some embodiments, the internal linker is in a nexus region of the gRNA. In some embodiments, the internal linker substitutes for 1 or 2 nucleotides of the nexus region of the gRNA.

In some embodiments, the internal linker is in a hairpin between a first portion of the gRNA and a second portion of the gRNA, wherein the first portion and the second portion together form a duplex portion. In some embodiments, the internal linker bridges a first portion of a duplex and a second portion of a duplex, wherein the duplex comprises 2-10 base pairs.

In some embodiments, the gRNA comprises two internal linkers. In some embodiments, the gRNA comprises three internal linkers.

In some embodiments, a single-guide RNA (sgRNA) is provided, the sgRNA comprising a guide region and a conserved portion at 3′ to the guide region, wherein the conserved portion comprises a repeat-anti-repeat region, a nexus region, a hairpin 1 region, and a hairpin 2 region, and comprises at least one of
1) a first internal linker substituting for at least 2 nucleotides of an upper stem region of the repeat-anti-repeat region;
2) a second internal linker substituting for 1 or 2 nucleotides of the nexus region; and
3) a third internal linker substituting for at least 2 nucleotides of the hairpin 1.

In some embodiments, a single-guide RNA (sgRNA) is provided, the sgRNA comprising a guide region and a conserved portion at the 3′ to the guide region, wherein conserved portion comprises a repeat-anti-repeat region, a hairpin 1 region, and a hairpin 2 region, and further comprises at least one of:
1) a first internal linker substituting for at least 2 nucleotides of an upper stem region of the repeat-anti-repeat region of the sgRNA;
2) a second internal linker substituting for 1 or 2 nucleotides of the hairpin 1 of the sgRNA; or
3) a third internal linker substituting for at least 2 nucleotides of the hairpin 2 of the sgRNA.

In some embodiments, a guide RNA (gRNA) is provided, the gRNA comprising a guide region and a conserved portion 3′ to the guide region, wherein the conserved portion comprises a repeat-anti-repeat region, a hairpin 1 region, and a hairpin 2 region, and comprises a first internal linker substituting for at least 2 nucleotides of the repeat-anti-repeat region and a second internal linker substituting for at least 2 nucleotides of the hairpin 2.

In some embodiments, a guide RNA (gRNA) is provided, the gRNA comprising a guide region and a conserved portion 3′ to the guide region, wherein the conserved portion comprises a repeat-anti-repeat region and a hairpin region, and comprises an internal linker substituting for at least 2 nucleotides of the repeat-anti-repeat region.

In some embodiments, a guide RNA (gRNA) is provided, the gRNA comprising a repeat-anti-repeat region, and an internal linker substituting for at least 2 nucleotides of the repeat-anti-repeat region.

In some embodiments, the internal linker comprises at least two ethylene glycol subunits covalently linked to each other.

The following is a non-exhaustive listing of embodiments provided herein.
Embodiment 1 is a guide RNA (gRNA) comprising an internal linker.
Embodiment 2 is the gRNA of embodiment 1, wherein the internal linker substitutes for at least 2, 3, 4, 5, 6, 7, 8, 9, 10, 11, 12, 13, 14, 15, 16, 17, 18, 19, 20, 21, 22, 23, 24, 25, 26, 27, or 28 nucleotides of the gRNA.
Embodiment 3 is the gRNA of embodiment 1 or 2 wherein the internal linker has a bridging length of about 3-30 atoms, optionally 12-21 atoms, and the linker substitutes for at least 2 nucleotides of the gRNA.
Embodiment 4 is the gRNA of any one of embodiments 1-3, wherein the internal linker has a bridging length of about 6-18 atoms, optionally about 6-12 atoms, and the linker substitutes for at least 2 nucleotides of the gRNA.
Embodiment 5 is the gRNA of any one of embodiments 1-4, wherein the internal linker substitutes for 2-12 nucleotides.
Embodiment 6 is the gRNA of any one of embodiments 1-5, wherein the internal linker is in a repeat-anti-repeat region of the gRNA.
Embodiment 7 is the gRNA of any one of embodiments 1-6, wherein the internal linker substitutes for at least 4 nucleotides of the repeat-anti-repeat region of the gRNA.
Embodiment 8 is the gRNA of any one of embodiments 1-7, wherein the internal linker substitutes for up to 28 nucleotides of the repeat-anti-repeat region of the gRNA.
Embodiment 9 is the gRNA of any one of embodiments 1-8, wherein the internal linker substitutes for 2, 3, 4, 5, 6, 7, 8, 9, 10, 11, or 12 nucleotides of the repeat-anti-repeat region of the gRNA.
Embodiment 10 is the gRNA of any one of embodiments 1-9, wherein the internal linker is in a hairpin region of the gRNA.
Embodiment 11 is the gRNA of any one of embodiments 1-10, wherein the internal linker substitutes for at least 2 nucleotides of the hairpin region of the gRNA.
Embodiment 12 is the gRNA of any one of embodiments 1-11, wherein the internal linker substitutes for up to 22 nucleotides of the hairpin region of the gRNA.
Embodiment 13 is the gRNA of any one of embodiments 1-12, wherein the internal linker substitutes for 2, 3, 4, 5, 6, 7, 8, 9, 10, 11, 12, 13, 14, 15, 16, 17, 18, 19, 20, 21, or 22 nucleotides of the hairpin region of the gRNA.
Embodiment 14 is the gRNA of any one of embodiments 1-13, wherein the internal linker substitutes for 1, 2, 3, 4, 5, 6, 7, 8, or 9 base pairs of the hairpin region of the gRNA.
Embodiment 15 is the gRNA of any one of embodiments 1-14, wherein the internal linker is in a nexus region of the gRNA.
Embodiment 16 is the gRNA of any one of embodiments 1-15, wherein the internal linker substitutes for 1 or 2 nucleotides of the nexus region of the gRNA.
Embodiment 17 is the gRNA of any one of embodiments 1-16, wherein the internal linker is in a hairpin between a first portion of the gRNA and a second portion of the gRNA, wherein the first portion and the second portion together form a duplex portion.
Embodiment 18 is the gRNA of any one of embodiments 1-17, wherein the internal linker bridges a first portion of a duplex and a second portion of a duplex, wherein the duplex comprises 2-10 base pairs.
Embodiment 19 is the gRNA of any one of embodiments 1-18, wherein the gRNA comprises two internal linkers.
Embodiment 20 is the gRNA of any one of embodiments 1-18, wherein the gRNA comprises three internal linkers.
Embodiment 21 is the gRNA of any one of embodiments 1-20, wherein the internal linker in the repeat-anti-repeat region is in a hairpin between a first portion and a second portion of the repeat-anti-repeat region, wherein the first portion and the second portion together form a duplex portion.
Embodiment 22 is the gRNA of embodiment 21, wherein the internal linker in the repeat-anti-repeat region substitutes for 2, 3, 4, 5, 6, 7, 8, 9, 10, 11, 12, 13, 14, 15, 16, 17, 18, 19, 20, 21, 22, 23, 24, 25, 26, 27, or 28 nucleotides of the hairpin.
Embodiment 23 is the gRNA of any one of embodiments 21-22, wherein the internal linker in the repeat-anti-repeat region substitutes for at least 4 nucleotides of the hairpin.
Embodiment 24 is the gRNA of any one of embodiments 21-23, wherein the internal linker in the repeat-anti-repeat region substitutes for up to 28 nucleotides of the hairpin.
Embodiment 25 is the gRNA of any one of embodiments 21-24, wherein the internal linker in the repeat-anti-repeat region substitutes for 4-20 nucleotides of the hairpin.
Embodiment 26 is the gRNA of any one of embodiments 21-25, wherein the internal linker in the repeat-anti-repeat region substitutes for 4-14 nucleotides of the hairpin.
Embodiment 27 is the gRNA of any one of embodiments 21-26, wherein the internal linker in the repeat-anti-repeat region substitutes for 4-6 nucleotides of the hairpin.
Embodiment 28 is the gRNA of any one of embodiments 21-27, wherein the internal linker in the repeat-anti-repeat region substitutes for a loop, or part thereof, of the hairpin.
Embodiment 29 is the gRNA of any one of embodiments 21-28, wherein the internal linker in the repeat-anti-repeat region substitutes for the loop and the stem, or part thereof, of the hairpin.
Embodiment 30 is the gRNA of any one of embodiments 21-27, wherein the internal linker in the repeat-anti-repeat region substitutes for 2, 3, or 4 nucleotides of the loop of the hairpin.
Embodiment 31 is the gRNA of any one of embodiments 21-27, wherein the internal linker in the repeat-anti-repeat region substitutes for the loop of the hairpin and at least 1 nucleotide of the stem of the hairpin.
Embodiment 32 is the gRNA of any one of embodiments 21-31, wherein the internal linker in the repeat-anti-repeat region substitutes for the loop of the hairpin and 1, 2, 3, 4, 5, 6, 7, 8, 9, 10, 11, 12, 13, 14, 15, 16, 17, 18, 19, 20, 21, 22, 23, or 24 nucleotides of the stem of the hairpin.
Embodiment 33 is the gRNA of any one of embodiments 21-32, wherein the internal linker in the repeat-anti-repeat region substitutes for the loop of the hairpin and at least 2 nucleotides of the stem of the hairpin.
Embodiment 34 is the gRNA of any one of embodiments 21-32, wherein the internal linker in the repeat-anti-repeat region substitutes for the loop of the hairpin and 1, 2, 3, 4, 5, 6, 7, or 8 nucleotides of the stem of the hairpin.
Embodiment 35 is the gRNA of any one of embodiments 21-32, wherein the internal linker in the repeat-anti-repeat region substitutes for the loop of the hairpin and 1, 2, 3, 4, 5, 6, 7, 8, 9, 10, 11, or 12 base pairs of the stem of the hairpin.
Embodiment 36 is the gRNA of any one of embodiments 21-32, wherein the internal linker in the repeat-anti-repeat region substitutes for all of the nucleotides constituting the loop of the hairpin.
Embodiment 37 is the gRNA of any one of embodiments 21-32, wherein the internal linker in the repeat-anti-repeat region substitutes for all of the nucleotides constituting the loop and the stem of the hairpin.
Embodiment 38 is the gRNA of any one of embodiments 1-37, wherein the internal linker substitutes for 1 or 2 nucleotides of the nexus region of the gRNA.
Embodiment 39 is the gRNA of any one of embodiments 1-38, wherein the internal linker substitutes for a hairpin of the gRNA.
Embodiment 40 is the gRNA of embodiment 39, wherein the internal linker substitutes for 2, 3, 4, 5, 6, 7, 8, 9, 10, 11, 12, 13, 14, 15, 16, 17, 18, 19, 20, 21, or 22 nucleotides of the hairpin.
Embodiment 41 is the gRNA of any one of embodiments 39-40, wherein the internal linker substitutes for 2-22 nucleotides of the hairpin.
Embodiment 42 is the gRNA of any one of embodiments 39-41, wherein the internal linker substitutes for 2-12 nucleotides of the hairpin.
Embodiment 43 is the gRNA of any one of embodiments 39-42, wherein the internal linker substitutes for 2-6 nucleotides of the hairpin.
Embodiment 44 is the gRNA of any one of embodiments 39-43, wherein the internal linker substitutes for 2-4 nucleotides of the hairpin.
Embodiment 45 is the gRNA of any one of embodiments 39-44, wherein the internal linker substitutes for a loop, or part thereof, of the hairpin.
Embodiment 46 is the gRNA of any one of embodiments 39-45, wherein the internal linker substitutes for the loop and the stem, or part thereof, of the hairpin.
Embodiment 47 is the gRNA of any one of embodiments 39-46, wherein the internal linker substitutes for 2, 3, 4, or 5 nucleotides of the loop of the hairpin.
Embodiment 48 is the gRNA of any one of embodiments 39-47, wherein the internal linker substitutes for the loop of the hairpin and at least 1 nucleotide of the stem of the hairpin.
Embodiment 49 is the gRNA of any one of embodiments 39-48, wherein the internal linker substitutes for the loop of the hairpin and 1, 2, 3, 4, 5, 6, 7, 8, 9, 10, 11, 12, 13, 14, 15, 16, 17, or 18 nucleotides of the stem of the hairpin.
Embodiment 50 is the gRNA of any one of embodiments 39-49, wherein the internal linker substitutes for the loop of the hairpin and at least 2 nucleotides of the stem of the hairpin.
Embodiment 51 is the gRNA of any one of embodiments 39-50, wherein the internal linker in the repeat-anti-repeat region substitutes for the loop of the hairpin and up to 18 nucleotides of the stem of the hairpin.
Embodiment 52 is the gRNA of any one of embodiments 39-51, wherein the internal linker in the repeat-anti-repeat region substitutes for the loop of the hairpin and 1, 2, 3, 4, 5, 6, 7, 8, or 9 base pairs of the stem of the hairpin.
Embodiment 53 is the gRNA of any one of embodiments 39-52, wherein the internal linker substitutes for all of the nucleotides constituting the loop of the hairpin.
Embodiment 54 is the gRNA of any one of embodiments 39-53, wherein the internal linker substitutes for all of the nucleotides constituting the loop and the stem of the hairpin.
Embodiment 55 is the gRNA of any one of embodiments 39-54, wherein the hairpin is a hairpin 1.
Embodiment 56 is the gRNA of any one of embodiments 39-54, wherein the hairpin is a hairpin 2.
Embodiment 57 is the gRNA of any one of embodiments 39-54, wherein the hairpin is a hairpin 1, and the internal linker substitutes for the hairpin 1.
Embodiment 58 is the gRNA of embodiment 57, wherein the gRNA further comprises a hairpin 2 at 3′ to the hairpin 1.
Embodiment 59 is the gRNA of embodiment 58, wherein the internal linker substitutes for at least 2 nucleotides of a loop of the hairpin 2.
Embodiment 60 is the gRNA of embodiment 58 or 59, wherein the internal linker does not substitute for the hairpin 2.
Embodiment 61 is the gRNA of any one of embodiments 1-60, further comprising a guide region.
Embodiment 62 is the gRNA of embodiment 61, wherein the guide region is 17, 18, 19, or 20 nucleotides in length.
Embodiment 63 is the gRNA of any one of embodiments 1-62, wherein the gRNA is a single guide RNA (sgRNA).
Embodiment 64 is the gRNA of any one of embodiments 1-62, wherein the gRNA comprises a tracrRNA (trRNA).
Embodiment 65 is a guide RNA (gRNA), wherein the gRNA is a single-guide RNA (sgRNA) comprising a guide region and a conserved portion at 3′ to the guide region, wherein the conserved portion comprises a repeat-anti-repeat region, a nexus region, a hairpin 1 region, and a hairpin 2 region, and comprises at least one of:
   1) a first internal linker substituting for at least 2 nucleotides of an upper stem region of the repeat-anti-repeat region;
   2) a second internal linker substituting for 1 or 2 nucleotides of the nexus region; and
   3) a third internal linker substituting for at least 2 nucleotides of the hairpin 1.
Embodiment 66 is the gRNA of embodiment 65, wherein the sgRNA comprises the first internal linker and the second internal linker.
Embodiment 67 is the gRNA of embodiment 65, wherein the sgRNA comprises the first internal linker and the third internal linker.
Embodiment 68 is the gRNA of embodiment 65, wherein the sgRNA comprises the second internal linker and the third internal linker.
Embodiment 69 is the gRNA of embodiment 65, wherein the sgRNA comprises the first internal linker, the second internal linker, and the third internal linker.
Embodiment 70 is the gRNA of any one of embodiments 65-69, wherein the first internal linker has a bridging length of about 9-30 atoms, optionally about 15-21 atoms.
Embodiment 71 is the gRNA of any one of embodiments 65-70, wherein the first internal linker substitutes for 2, 3, 4, 5, 6, 7, 8, 9, 10, 11, or 12 nucleotides of the upper stem region.
Embodiment 72 is the gRNA of any one of embodiments 65-71, wherein the first internal linker substitutes for a loop, or part thereof, of the upper stem region.
Embodiment 73 is the gRNA of any one of embodiments 65-72, wherein the first internal linker substitutes for the loop and the stem, or part thereof, of the upper stem region.
Embodiment 74 is the gRNA of any one of embodiments 65-73, wherein the first internal linker substitutes for 2, 3, or 4 nucleotides of the loop of the upper stem region.
Embodiment 75 is the gRNA of any one of embodiments 65-74, wherein the first internal linker substitutes for the loop of the upper stem region and at least 2, 3, 4, 5, 6, 7, or 8 nucleotides of the stem of the upper stem region.
Embodiment 76 is the gRNA of any one of embodiments 65-75, wherein the first internal linker substitutes for the loop of the upper stem region and 1, 2, 3, or 4 base pairs of the stem of the upper stem region.
Embodiment 77 is the gRNA of any one of embodiments 65-76, wherein the first internal linker substitutes for all of the nucleotides constituting the loop of the upper stem region.
Embodiment 78 is the gRNA of any one of embodiments 65-77, wherein the first internal linker substitutes for all of the nucleotides constituting the loop and the stem of the upper stem region.
Embodiment 79 is the gRNA of any one of embodiments 65-78, wherein the second internal linker has a bridging length of about 6-18 atoms, optionally about 6-12 atoms.
Embodiment 80 is the gRNA of any one of embodiments 65-79, wherein the second internal linker substitutes for 2 nucleotides of the nexus region of the sgRNA.
Embodiment 81 is the gRNA of any one of embodiments 65-80, wherein the second internal linker substitutes for 2 nucleotides of a loop of the nexus region of the sgRNA.
Embodiment 82 is the gRNA of any one of embodiments 65-81, wherein the third internal linker has a bridging length of about 9-30, optionally about 12-21 atoms.
Embodiment 83 is the gRNA of any one of embodiments 65-82, wherein the third internal linker substitutes for 2, 3, 4, 5, 6, 7, 8, 9, 10, 11 or 12 nucleotides of the hairpin 1 of the gRNA.
Embodiment 84 is the gRNA of any one of embodiments 65-83, wherein the third linker substitutes for 1, 2, 3, 4, or 5 base pairs of the hairpin 1 of the gRNA.
Embodiment 85 is the gRNA of any one of embodiments 65-84, wherein the third internal linker substitutes for a loop, or part thereof, of the hairpin 1.
Embodiment 86 is the gRNA of any one of embodiments 65-85, wherein the third internal linker substitutes for the loop and the stem, or part thereof, of the hairpin 1.
Embodiment 87 is the gRNA of any one of embodiments 65-86, wherein the third internal linker substitutes for 2, 3, or 4 nucleotides of the loop of the hairpin 1.
Embodiment 88 is the gRNA of any one of embodiments 65-87, wherein the third internal linker substitutes for the loop of the hairpin and at least 1 nucleotide of the stem of the hairpin 1.
Embodiment 89 is the gRNA of any one of embodiments 65-88, wherein the third internal linker substitutes for the loop of the hairpin and 2, 4, or 6 nucleotides of the stem of the hairpin 1.
Embodiment 90 is the gRNA of any one of embodiments 65-89, wherein the third internal linker in the repeat-anti-repeat region substitutes for the loop of the hairpin and 1, 2, or 3 base pairs of the stem of the hairpin 1.
Embodiment 91 is the gRNA of any one of embodiments 65-90, wherein the third internal linker substitutes for all of the nucleotides constituting the loop of the hairpin 1.
Embodiment 92 is the gRNA of any one of embodiments 65-91, wherein the third internal linker substitutes for all of the nucleotides constituting the loop and the stem of the hairpin 1.
Embodiment 93 is the gRNA of any one of embodiments 65-92, wherein the hairpin 2 region of the sgRNA does not contain any internal linker.
Embodiment 94 is the gRNA of any one of embodiments 65-93, wherein the sgRNA is an S. pyogenes Cas9 sgRNA.
Embodiment 95 is the gRNA of any one of embodiments 65-94, wherein the sgRNA comprises a conserved portion comprising a sequence of SEQ ID NO: 400.
Embodiment 96 is the gRNA of embodiment 95, wherein 2, 3 or 4 of nucleotides 13-16 (US5-US8 of the upper stem region) are substituted for the first internal linker relative to SEQ ID NO: 400.
Embodiment 97 is the gRNA of any one of embodiments 95-96, wherein nucleotides 12-17 (US4-US9 of the upper stem region) are substituted for the first internal linker relative to SEQ ID NO: 400.
Embodiment 98 is the gRNA of any one of embodiments 95-97, wherein d nucleotides to 11-18 (US3-US10 of the upper stem region) are substituted for the first internal linker relative to SEQ ID NO: 400.
Embodiment 99 is the gRNA of any one of embodiments 95-98, wherein nucleotides to 10-19 (US2-US11 of the upper stem region) are substituted for the first internal linker relative to SEQ ID NO: 400.
Embodiment 100 is the gRNA of any one of embodiments 95-99, wherein nucleotides to 9-20 (US1-US12 of the upper stem region) are substituted for the first internal linker relative to SEQ ID NO: 400.
Embodiment 101 is the gRNA of any one of embodiments 95-100, wherein nucleotide 36-37 (N6-N7 of the nexus region) are substituted for the second internal linker relative to SEQ ID NO: 400.
Embodiment 102 is the gRNA of any one of embodiments 95-101, wherein 2, 3, or 4 of nucleotides 53-56 (H1-5-H1-8 of the hairpin 1) are substituted for the third internal linker relative to SEQ ID NO: 400.
Embodiment 103 is the gRNA of any one of embodiments 95-102, wherein nucleotides 52-57 (H1-4-H1-9 of the hairpin 1) are substituted for the third internal linker relative to SEQ ID NO: 400.
Embodiment 104 is the gRNA of any one of embodiments 95-103, wherein nucleotides 51-58 (H1-3-H1-10 of the hairpin 1) are substituted for the third internal linker relative to SEQ ID NO: 400.
Embodiment 105 is the gRNA of any one of embodiments 95-104, wherein nucleotides 50-59 (H1-1-H1-12 of the hairpin 1) are substituted for the third internal linker relative to SEQ ID NO: 400.
Embodiment 106 is the gRNA of any one of embodiments 95-105, wherein nucleotides 77-80 are deleted relative to SEQ ID NO: 400.
Embodiment 107 is the gRNA of any one of embodiments 65-94, wherein the sgRNA comprises a sequence of SEQ ID NO: 201.
Embodiment 108 is the gRNA of embodiment 107, wherein 2, 3 or 4 of nucleotides 33-36 are substituted for the first internal linker relative to SEQ ID NO: 201.
Embodiment 109 is the gRNA of any one of embodiments 107-108, wherein nucleotides 32-37 are substituted for the first internal linker relative to SEQ ID NO: 201.
Embodiment 110 is the gRNA of any one of embodiments 107-109, wherein nucleotides 31-38 are substituted for the first internal linker relative to SEQ ID NO: 201.
Embodiment 111 is the gRNA of any one of embodiments 107-110, wherein nucleotides 30-39 are substituted for the first internal linker relative to SEQ ID NO: 201.
Embodiment 112 is the gRNA of any one of embodiments 107-111, wherein nucleotides 29-40 are substituted for the first internal linker relative to SEQ ID NO: 201.
Embodiment 113 is the gRNA of any one of embodiments 107-112, wherein nucleotide 55-56 are substituted for the second internal linker relative SEQ ID NO: 201.
Embodiment 114 is the gRNA of any one of embodiments 107-113, wherein 2, 3, or 4 of nucleotides 50-53 are substituted for the third internal linker relative to SEQ ID NO: 201.
Embodiment 115 is the gRNA of any one of embodiments 107-114, wherein nucleotides 49-54 are substituted for the third internal linker relative to SEQ ID NO: 201.
Embodiment 116 is the gRNA of any one of embodiments 107-115, wherein nucleotides 77-80 are deleted relative to SEQ ID NO: 201.
Embodiment 117 is a guide RNA (gRNA), wherein the gRNA is a single-guide RNA (sgRNA) comprising a guide region and a conserved portion at the 3′ to the guide region, wherein conserved portion comprises a repeat-anti-repeat region, a hairpin 1 region, and a hairpin 2 region, and further comprises at least one of:
   1) a first internal linker substituting for at least 2 nucleotides of an upper stem region of the repeat-anti-repeat region of the sgRNA;
   2) a second internal linker substituting for 1 or 2 nucleotides of the hairpin 1 of the sgRNA; or
   3) a third internal linker substituting for at least 2 nucleotides of the hairpin 2 of the sgRNA.
Embodiment 118 is the gRNA of embodiment 117, wherein the sgRNA comprises the first internal linker and the second internal linker.
Embodiment 119 is the gRNA of embodiment 117, wherein the sgRNA comprises the first internal linker and the third internal linker.
Embodiment 120 is the gRNA of embodiment 117, wherein the sgRNA comprises the second internal linker and the third internal linker.
Embodiment 121 is the gRNA of embodiment 117, wherein the sgRNA comprises the first internal linker, the second internal linker, and the third internal linker.
Embodiment 122 is the gRNA of any one of embodiments 117-121, wherein the first internal linker has a bridging length of about 9-30 atoms, optionally about 15-21 atoms.
Embodiment 123 is the gRNA of any one of embodiments 117-122, wherein the first internal linker is in a hairpin between a first portion of the sgRNA and a second portion of the sgRNA, wherein the first portion and the second portion together form a duplex portion.
Embodiment 124 is the gRNA of any one of embodiments 117-123, wherein the first internal linker substitutes for 2, 3, 4, 5, 6, 7, 8, 9, 10, 11, or 12 nucleotides of the upper stem region.
Embodiment 125 is the gRNA of any one of embodiments 117-124, wherein the first internal linker substitutes for a loop, or part thereof, of the upper stem region.
Embodiment 126 is the gRNA of any one of embodiments 117-125, wherein the first internal linker substitutes for the loop and the stem, or part thereof, of the upper stem region.
Embodiment 127 is the gRNA of any one of embodiments 117-126, wherein the first internal linker substitutes for 2, 3, or 4 nucleotides of the loop of the upper stem region.
Embodiment 128 is the gRNA of any one of embodiments 117-127, wherein the first internal linker substitutes for the loop of the upper stem region and at least 2, 4, 6, or 8 nucleotides of the stem of the upper stem region.
Embodiment 129 is the gRNA of any one of embodiments 117-128, wherein the first internal linker substitutes for the loop of the upper stem region and 1, 2, 3, or 4 base pairs of the stem of the upper stem region.
Embodiment 130 is the gRNA of any one of embodiments 117-129, wherein the first internal linker substitutes for all of the nucleotides constituting the loop of the upper stem region.
Embodiment 131 is the gRNA of any one of embodiments 117-130, wherein the first internal linker substitutes for all of the nucleotides constituting the loop and the stem of the upper stem region.
Embodiment 132 is the gRNA of any one of embodiments 117-131, wherein the second internal linker has a bridging length of about 6-18 atoms, optionally about 6-12 atoms.
Embodiment 133 is the gRNA of any one of embodiments 117-132, wherein the second internal linker substitutes for 2 nucleotides of the hairpin 1 of the sgRNA.
Embodiment 134 is the gRNA of any one of embodiments 117-133, wherein the second internal linker substitutes for 2 nucleotides of a stem region of the nexus region of the sgRNA.
Embodiment 135 is the gRNA of any one of embodiments 117-134, wherein the third internal linker has a bridging length of about 9-30, optionally about 12-21 atoms.
Embodiment 136 is the gRNA of any one of embodiments 117-135, wherein the third internal linker substitutes for 4, 6, 8, 10, or 12 nucleotides of the hairpin 2 of the gRNA.
Embodiment 137 is the gRNA of any one of embodiments 117-136, wherein the third linker substitutes for 1, 2, 3, 4, or 5 base pairs of the hairpin 2 of the gRNA.
Embodiment 138 is the gRNA of any one of embodiments 117-137, wherein the third internal linker substitutes for a loop, or part thereof, of the hairpin 2.
Embodiment 139 is the gRNA of any one of embodiments 117-138, wherein the third internal linker substitutes for the loop and the stem, or part thereof, of the hairpin 2.
Embodiment 140 is the gRNA of any one of embodiments 117-139, wherein the third internal linker substitutes for 2, 3, or 4 nucleotides of the loop of the hairpin 2.
Embodiment 141 is the gRNA of any one of embodiments 117-140, wherein the third internal linker substitutes for the loop of the hairpin and at least 1 nucleotide of the stem of the hairpin 2.
Embodiment 142 is the gRNA of any one of embodiments 117-141, wherein the third internal linker substitutes for the loop of the hairpin and 2, 4, or 6 nucleotides of the stem of the hairpin 2.
Embodiment 143 is the gRNA of any one of embodiments 117-142, wherein the third internal linker in the repeat-anti-repeat region substitutes for the loop of the hairpin and 1, 2, or 3 base pairs of the stem of the hairpin 2.
Embodiment 144 is the gRNA of any one of embodiments 117-143, wherein the third internal linker substitutes for all of the nucleotides constituting the loop of the hairpin 2.
Embodiment 145 is the gRNA of any one of embodiments 117-144, wherein the third internal linker is in a hairpin between a first portion of the sgRNA and a second portion of the sgRNA, wherein the first portion and the second portion together form a duplex portion.
Embodiment 146 is the gRNA of any one of embodiments 117-145, wherein the gRNA is a S. aureus Cas9 (SauCas9) guide RNA, and does not include the third internal linker.
Embodiment 147 is the gRNA of any one of embodiments 117-146, wherein the gRNA is a C. diphtheriae Cas9 (CdiCas9) guide RNA, an S. thermophilus Cas9 (St1Cas9) guide RNA, or an Acidothermus cellulolyticus Cas9 (AceCas9) guide RNA.
Embodiment 148 is the gRNA of any one of embodiments 117-147, wherein the sgRNA comprises a sequence of SEQ ID NO: 202.
Embodiment 149 is the gRNA of embodiment 148, wherein 22, 3 or 4 of nucleotides 35-38 are substituted for the first internal linker relative SEQ ID NO: 202.
Embodiment 150 is the gRNA of any one of embodiments 148-149, wherein nucleotides 34-39 are substituted for the first internal linker relative SEQ ID NO: 202.
Embodiment 151 is the gRNA of any one of embodiments 148-150, wherein nucleotides 33-40 are substituted for the first internal linker relative SEQ ID NO: 202.
Embodiment 152 is the gRNA of any one of embodiments 148-151, wherein nucleotides 32-41 are substituted for the first internal linker relative SEQ ID NO: 202.
Embodiment 153 is the gRNA of any one of embodiments 148-152, wherein nucleotides 31-42 are substituted for the first internal linker relative SEQ ID NO: 202.
Embodiment 154 is the gRNA of any one of embodiments 148-153, wherein nucleotide 61-62 are substituted for the second internal linker relative SEQ ID NO: 202.
Embodiment 155 is the gRNA of any one of embodiments 148-154, wherein 2, 3, or 4 of nucleotides 84-87 are substituted for the third internal linker relative SEQ ID NO: 202.
Embodiment 156 is the gRNA of any one of embodiments 148-155, wherein nucleotides 83-88 are substituted for the third internal linker relative SEQ ID NO: 202.
Embodiment 157 is the gRNA of any one of embodiments 148-156, wherein nucleotides 82-89 are substituted for the third internal linker relative SEQ ID NO: 202.
Embodiment 158 is the gRNA of any one of embodiments 148-157, wherein nucleotides 81-90 are substituted for the third internal linker relative SEQ ID NO: 202.
Embodiment 159 is the gRNA of any one of embodiments 148-158, wherein nucleotides 97-100 are deleted relative SEQ ID NO: 202.
Embodiment 160 is a guide RNA (gRNA) comprising a guide region and a conserved portion 3′ to the guide region, wherein the conserved portion comprises a repeat-anti-repeat region, a hairpin 1 region, and a hairpin 2 region, and comprises a first internal linker substituting for at least 2 nucleotides of the repeat-anti-repeat region and a second internal linker substituting for at least 2 nucleotides of the hairpin 2.
Embodiment 161 is the gRNA of embodiment 160, wherein the first internal linker has a bridging length of about 9-30 atoms, optionally about 15-21 atoms.
Embodiment 162 is the gRNA of any one of embodiments 160-161, wherein the first internal linker substitutes for 2, 3, 4, 5, 6, 7, 8, 9, 10, 11, 12, 13, 14, 15, 16, 17, or 18 nucleotides of the repeat-anti-repeat region of the gRNA.
Embodiment 163 is the gRNA of any one of embodiments 160-162, wherein the first internal linker is in a hairpin between a first portion of the sgRNA and a second portion of the repeat-anti-repeat region, wherein the first portion and the second portion together form a duplex portion.
Embodiment 164 is the gRNA of any one of embodiments 160-163, wherein the first internal linker substitutes for a loop, or part thereof, of the hairpin of the repeat-anti-repeat region.
Embodiment 165 is the gRNA of any one of embodiments 160-164, wherein the first internal linker substitutes for the loop and the stem, or part thereof, of the hairpin of the repeat-anti-repeat region.
Embodiment 166 is the gRNA of any one of embodiments 160-165, wherein the first internal linker substitutes for 1, 2, 3, or 4 nucleotides of the loop of the hairpin of the repeat-anti-repeat region.
Embodiment 167 is the gRNA of any one of embodiments 160-166, wherein the first internal linker substitutes for the loop of the hairpin and at least 1, 2, 3, 4, 5, 6, 7, 8, 9, 10, 11, 12, 13, or 14 nucleotides of the upper stem of the hairpin of the repeat-anti-repeat region.
Embodiment 168 is the gRNA of any one of embodiments 160-167, wherein the first internal linker substitutes for the loop of the hairpin and 1, 2, 3, 4, 5, 6, or 7 base pairs of the upper stem of the hairpin of the repeat-anti-repeat region.
Embodiment 169 is the gRNA of any one of embodiments 160-168, wherein the first internal linker substitutes for all of the nucleotides constituting the loop of the hairpin of the repeat-anti-repeat region.
Embodiment 170 is the gRNA of any one of embodiments 160-169, wherein the first internal linker substitutes for all of the nucleotides constituting the loop and the upper stem of the hairpin of the repeat-anti-repeat region.
Embodiment 171 is the gRNA of any one of embodiments 160-169, wherein the first internal linker substitutes for all of the nucleotides constituting the loop of the repeat-anti-repeat region; and the upper stem of the hairpin of the repeat-anti-repeat region comprises at least one base pair, or no more than one, two, or three base pairs.
Embodiment 172 is the gRNA of any one of embodiments 160-171, wherein the second internal linker has a bridging length of about 9-30, optionally about 12-21 atoms.
Embodiment 173 is the gRNA of any one of embodiments 160-172, wherein the second internal linker substitutes for 2, 3, 4, 5, 6, 7, 8, 9, or 10 nucleotides of the hairpin 2 of the gRNA.
Embodiment 174 is the gRNA of any one of embodiments 160-173, wherein the second internal linker substitutes for a loop region of the hairpin 2.
Embodiment 175 is the gRNA of any one of embodiments 160-174, wherein the second internal linker substitutes for a loop region and part of a stem region of the hairpin 2.
Embodiment 176 is the gRNA of any one of embodiments 160-175, wherein the second internal linker substitutes for a loop, or part thereof, of the hairpin 2.
Embodiment 177 is the gRNA of any one of embodiments 160-176, wherein the second internal linker substitutes for the loop and the stem, or part thereof, of the hairpin 2.
Embodiment 178 is the gRNA of any one of embodiments 160-177, wherein the second internal linker substitutes for 2, 3, or 4 nucleotides of the loop of the hairpin 2.
Embodiment 179 is the gRNA of any one of embodiments 160-178, wherein the second internal linker substitutes for all of the nucleotides constituting the loop of the hairpin 2.
Embodiment 180 is the gRNA of any one of embodiments 160-179, wherein the second internal linker substitutes for the loop of the hairpin 2 and at least 1, 2, 3, 4, 5, or 6 nucleotides of the stem of the hairpin 2.
Embodiment 181 is the gRNA of any one of embodiments 160-180, wherein the second internal linker substitutes for the loop of the hairpin and 1, 2, or 3 base pairs of the stem of the hairpin 2
Embodiment 182 is the gRNA of any one of embodiments 160-181, wherein the gRNA is a St1Cas9 guide RNA.
Embodiment 183 is the gRNA of any one of embodiments 160-182, wherein the sgRNA comprises a sequence of SEQ ID NO: 204.
Embodiment 184 is the gRNA of embodiment 183, wherein nucleotides 41-44 are substituted for the first internal linker relative SEQ ID NO: 204.
Embodiment 185 is the gRNA of any one of embodiments 183-184, wherein nucleotides 101-103 are substituted for the second internal linker relative SEQ ID NO: 204.
Embodiment 186 is the gRNA of any one of embodiments 183-185, wherein nucleotides 100-104 are substituted for the second internal linker relative SEQ ID NO: 204.
Embodiment 187 is the gRNA of any one of embodiments 183-186, wherein nucleotides 99-105 are substituted for the second internal linker relative SEQ ID NO: 204.
Embodiment 188 is the gRNA of any one of embodiments 183-187, wherein nucleotides 98-106 are substituted for the second internal linker relative SEQ ID NO: 204.
Embodiment 189 is the gRNA of any one of embodiments 183-188, wherein 2-18 nucleotides within nucleotides 94-111 are substituted relative to SEQ ID NO: 204.
Embodiment 190 is a guide RNA (gRNA) comprising a guide region and a conserved portion 3′ to the guide region, wherein the conserved portion comprises a repeat-anti-repeat region and a hairpin region, and comprises an internal linker substituting for at least 2 nucleotides of the repeat-anti-repeat region.
Embodiment 191 is the gRNA of embodiment 190, wherein the first internal linker has a bridging length of about 9-30 atoms, optionally about 12-21 atoms.
Embodiment 192 is the gRNA of any one of embodiments 190 or 191, wherein the first internal linker substitutes for 2, 3, 4, 5, 6, 7, 8, 9, 10, 11, or 12 nucleotides of the repeat-anti-repeat region of the gRNA.
Embodiment 193 is the gRNA of any one of embodiments 190-192, wherein the first internal linker is in a hairpin between a first portion of the sgRNA and a second portion of the repeat-anti-repeat region, wherein the first portion and the second portion together form a duplex portion.
Embodiment 194 is the gRNA of any one of embodiments 190-193, wherein the gRNA is a C. jejuni Cas9 (CjeCas9) guide RNA.
Embodiment 195 is the gRNA of any one of embodiments 190-194, wherein the gRNA is a CjeCas9 guide RNA and the internal linker is present only in the repeat-anti-repeat region of the gRNA.
Embodiment 196 is the gRNA of any one of embodiments 190-195, wherein the sgRNA comprises a sequence of SEQ ID NO: 207.
Embodiment 197 is the gRNA of embodiment 196, wherein nucleotides 33-36 are substituted for the internal linker relative SEQ ID NO: 207.
Embodiment 198 is the gRNA of any one of embodiments 196-197, wherein 1, 2, 3, 4, 5 or 6 base pairs of nucleotides 27-32 and 37-42 are substituted for the internal linker relative SEQ ID NO: 207.
Embodiment 199 is the gRNA of any one of embodiments 190-193, wherein the gRNA is a Francisella novicida Cas9 (FnoCas9) guide RNA.
Embodiment 200 is the gRNA of embodiment 199, wherein the sgRNA comprises a sequence of SEQ ID NO: 208.
Embodiment 201 is the gRNA of embodiment 200, wherein 2, 3 or 4 of nucleotides 40-43 are substituted for the internal linker relative SEQ ID NO: 208.
Embodiment 202 is the gRNA of any one of embodiments 200-201, wherein nucleotides 39-44 are substituted for the internal linker relative SEQ ID NO: 208.
Embodiment 203 is a guide RNA (gRNA) comprising a repeat-anti-repeat region, and an internal linker substituting for at least 2 nucleotides of the repeat-anti-repeat region.
Embodiment 204 is the gRNA of embodiment 203, wherein the internal linker has a bridging length of about 9-30 atoms, optionally about 15-21 atoms.
Embodiment 205 is the gRNA of any one of embodiments 203-204, wherein the internal linker substitutes for 2, 3, 4, 5, or 6 nucleotides of the repeat-anti-repeat region of the gRNA.
Embodiment 206 is the composition of any one of embodiments 203-205, wherein the gRNA is a Cpf1 guide RNA.
Embodiment 207 is the composition of embodiment 206, wherein the Cpf1 guide RNA is a Lachnospiraceae bacterium Cpf1 (LbCpf1) guide RNA, or a Acidaminococcus sp. Cpf1 (AsCpf1) guide RNA.
Embodiment 208 is the gRNA of any one of embodiments 203-207, wherein the sgRNA comprises a sequence of SEQ ID NO: 209 and nucleotides 11-14, or 12-15, or optionally 12-14, are substituted for the internal linker relative SEQ ID NO: 209.
Embodiment 209 is the composition of any one of embodiment 203-205, wherein the guide RNA is an Eubacterium siraeum (EsCas13d) guide RNA.
Embodiment 210 is the gRNA of any one of embodiments 203-205, and 209, wherein the sgRNA comprises a sequence of SEQ ID NO: 210 and nucleotides 9-16, or optionally 10-15, or at least 2 nucleotides thereof; are substituted for the internal linker relative to SEQ ID NO: 210.
Embodiment 211 is the gRNA of embodiment 1, wherein the internal linker is a first internal linker, second internal linker, or third internal linker; and the gRNA comprises a guide region and a conserved region comprising one or more of:
   (a) a shortened repeat/anti-repeat region, wherein the shortened repeat/anti-repeat region lacks 2-24 nucleotides, wherein
   (i) one or more of nucleotides 37-64 is deleted and optionally substituted relative to SEQ ID NO: 500; and
   (ii) nucleotide 36 is linked to nucleotide 65 by (i) a first internal linker that alone or in combination with nucleotides substitutes for 4 nucleotides, or (ii) at least 4 nucleotides; or
      (b) a shortened hairpin 1 region, wherein the shortened hairpin 1 lacks 2-10, optionally 2-8 nucleotides, wherein
   (i) one or more of nucleotides 82-95 is deleted and optionally substituted relative to SEQ ID NO: 500; and
   (ii) nucleotide 81 is linked to nucleotide 96 by (i) a second internal linker that alone or in combination with nucleotides substitutes for 4 nucleotides, or (ii) at least 4 nucleotides; or
      (c) a shortened hairpin 2 region, wherein the shortened hairpin 2 lacks 2-18, optionally 2-16 nucleotides, wherein
   (i) one or more of nucleotides 113-134 is deleted and optionally substituted relative to SEQ ID NO: 500; and
      (ii) nucleotide 112 is linked to nucleotide 135 by (i) a third internal linker that alone or in combination with nucleotides substitutes for 4 nucleotides, or (ii) at least 4 nucleotides;
   wherein one or both nucleotides 144-145 are optionally deleted as compared to SEQ ID NO: 500.
Embodiment 212 is a guide RNA (gRNA) comprising a guide region and a conserved region comprising one or more of:
   (a) a shortened repeat/anti-repeat region, wherein the shortened repeat/anti-repeat region lacks 2-24 nucleotides, wherein
      (i) one or more of nucleotides 37-64 is deleted and optionally substituted relative to SEQ ID NO: 500; and
      (ii) nucleotide 36 is linked to nucleotide 65 by (i) a first internal linker that alone or in combination with nucleotides substitutes for 4 nucleotides, or (ii) at least 4 nucleotides; or
   (b) a shortened hairpin 1 region, wherein the shortened hairpin 1 lacks 2-10, optionally 2-8 nucleotides, wherein
      (i) one or more of nucleotides 82-95 is deleted and optionally substituted relative to SEQ ID NO: 500; and
      (ii) nucleotide 81 is linked to nucleotide 96 by (i) a second internal linker that alone or in combination with nucleotides substitutes for 4 nucleotides, or (ii) at least 4 nucleotides; or
   (c) a shortened hairpin 2 region, wherein the shortened hairpin 2 lacks 2-18, optionally 2-16 nucleotides, wherein
      (i) one or more of nucleotides 113-134 is deleted and optionally substituted relative to SEQ ID NO: 500; and
      (ii) nucleotide 112 is linked to nucleotide 135 by (i) a third internal linker that alone or in combination with nucleotides substitutes for 4 nucleotides, or (ii) at least 4 nucleotides;
   wherein one or both nucleotides 144-145 are optionally deleted as compared to SEQ ID NO: 500;
   wherein the gRNA comprises at least one of the first internal linker, the second internal linker, and the third internal linker.
Embodiment 213 is the gRNA of embodiment 211 or 212, wherein the gRNA comprises at least two of the first internal linker, the second internal linker, and the third internal linker.
Embodiment 214 is the gRNA of any one of embodiments 211-213, wherein the gRNA comprises the first internal linker, the second internal linker, and the third internal linker.
Embodiment 215 is the gRNA of any one of embodiments 211-214, wherein at least 10 nucleotides are modified nucleotides.
Embodiment 216 is the gRNA of any one of embodiments 211-215, wherein the guide region has (i) an insertion of one nucleotide or a deletion of 1-4 nucleotides within positions 1-24 relative to SEQ ID NO: 500, or (ii) a length of 24 nucleotides.
Embodiment 217 is the gRNA of any one of embodiments 211-216, wherein the guide region has a length of 25, 24, 23, 22, 21, or 20 nucleotides, optionally wherein the guide region has a length of 25, 24, 23, or 22 nucleotides at positions 1-24 of SEQ ID NO: 500.
Embodiment 218 is the gRNA of embodiment 217, wherein the guide region has a length of 23 or 24 nucleotides at positions 1-24 of SEQ ID NO: 500.
Embodiment 219 is the gRNA of any one of embodiments 211-218, wherein the gRNA further comprises a 3′ tail.
Embodiment 220 is the gRNA of embodiment 219, wherein the 3′ tail comprises 1, 2, 3, 4, 5, 6, 7, 8, 9, or 10 nucleotides.
Embodiment 221 is the gRNA of embodiment 220, wherein the 3′ tail comprises 1, 2, 3, 4, or 5 nucleotides.
Embodiment 222 is the gRNA of any one of embodiments 219-221, wherein the 3′ tail terminates with a nucleotide comprising a uracil or a modified uracil.
Embodiment 223 is the gRNA of any one of embodiments 219-222, wherein the 3′ tail is 1 nucleotide in length.
Embodiment 224 is the gRNA of any one of embodiments 219-223, wherein the 3′ tail consists of a nucleotide comprising a uracil or a modified uracil.
Embodiment 225 is the gRNA of any one of embodiments 219-224, wherein the 3′ tail comprises a modification of any one or more of the nucleotides present in the 3′ tail.
Embodiment 226 is the gRNA of any one of embodiments 219-225, wherein the modification of the 3′ tail is one or more of 2′-O-methyl (2′-OMe) modified nucleotide and a phosphorothioate (PS) linkage between nucleotides.
Embodiment 227 is the gRNA of any one of the preceding embodiments 219-226, wherein the 3′ tail is fully modified.
Embodiment 228 is the gRNA of any one of embodiments 211-227, wherein the 3′ nucleotide of the gRNA is a nucleotide comprising a uracil or a modified uracil.
Embodiment 229 is the gRNA of any one of embodiments 211-228, wherein one or more of nucleotides 144 and 145 are deleted relative to SEQ ID NO: 500.
Embodiment 230 is the gRNA of any one of embodiments 211-229, wherein both nucleotides 144 and 145 are deleted relative to SEQ ID NO: 500.
Embodiment 231 is the gRNA of any one of embodiments 211-218, wherein the gRNA does not comprise a 3′ tail.
Embodiment 232 is the gRNA of any one of embodiments 211-231, wherein the shortened repeat/anti-repeat region lacks 2-28 nucleotides.
Embodiment 233 is the gRNA of any one of embodiments 211-232, wherein the shortened repeat/anti-repeat region has a length of 24, 25, 26, 27, 28, 29, 30, 31, 32, 33, 34, 35, 36, 37, 38, 39, 40, 41, 42, 43, 44, 45, 46, 47, 48, 49, or 50 nucleotides.
Embodiment 234 is the gRNA of any one of embodiments 211-233, wherein the shortened repeat/anti-repeat region lacks 12-28, optionally 18-24 nucleotides.
Embodiment 235 is the gRNA of any one of embodiments 211-234, wherein the shortened repeat/anti-repeat region has a length of 24, 25, 26, 27, 28, 29, 30, 31, 32, 33, 34, 35, 36, 37, 38, 39, or 40 nucleotides.
Embodiment 236 is the gRNA of any one of embodiments 211-235, wherein the shortened repeat/anti-repeat region has a length of 24, 25, 26, 27, 28, 29, 30, 31, 32, 33, or 34 nucleotides.
Embodiment 237 is the gRNA of any one of embodiments 211-236, wherein nucleotides 37-64 of SEQ ID NO: 500 form the upper stem, and one or more base pairs of the upper stem of the shortened repeat/anti-repeat region are deleted.
Embodiment 238 is the gRNA of any one of embodiments 211-237, wherein the upper stem of the shortened repeat/anti-repeat region comprises no more than one, two, three, or four base pairs.
Embodiment 239 is the gRNA of any one of embodiments 211-238, wherein all of positions 39-48 and all of positions 53-62 of the upper stem of the shortened repeat/anti-repeat region are deleted, and optionally nucleotide 38 or 63 is substituted.
Embodiment 240 is the gRNA of any one of embodiments 211-239, wherein all of positions 38-63 of the upper stem of the shortened repeat/anti-repeat region are deleted, and optionally nucleotide 37 or 64 is substituted.
Embodiment 241 is the gRNA of any one of embodiments 211-240, wherein all of nucleotides 37-64 of the upper stem of the shortened repeat/anti-repeat region are deleted, and optionally nucleotide 36 or 65 is substituted.
Embodiment 242 is the gRNA of any one of embodiments 211-241, wherein the shortened repeat/anti-repeat region has a duplex portion 11 base paired nucleotides in length.
Embodiment 243 is the gRNA of any one of embodiments 211-242, wherein the shortened repeat/anti-repeat region has a single duplex portion.
Embodiment 244 is the gRNA of any one of embodiments 211-243, wherein the upper stem of the shortened repeat/anti-repeat region includes one or more substitution relative to SEQ ID NO: 500.
Embodiment 245 is the gRNA of any one of embodiments 211-244, wherein the first internal linker substitutes for at least part of or for all of nucleotides 49-52.
Embodiment 246 is the gRNA of any one of embodiments 211-245, wherein all of nucleotides 37-64 are deleted and the first linker directly links nucleotide 36 to nucleotide 65.
Embodiment 247 is the gRNA of any one of embodiments 211-245, wherein all of nucleotides 38-63 are deleted and the first linker directly links nucleotide 37 to nucleotide 64.
Embodiment 248 is the gRNA of any one of embodiments 211-245, wherein all of nucleotides 39-62 are deleted and the first linker directly links nucleotide 38 to nucleotide 63.
Embodiment 249 is the gRNA of any one of embodiments 211-248, wherein the shortened repeat/anti-repeat region has 8-22 modified nucleotides.
Embodiment 250 is the gRNA of any one of embodiments 211-249, wherein the shortened hairpin 1 region lacks 2-10, optionally 2-8 or 2-4 nucleotides.
Embodiment 251 is the gRNA of any one of embodiments 211-250, wherein the shortened hairpin 1 region has a length of 9, 10, 11, 12, 13, 14, 15, 16, 17, 18, 19, 20 or 21 nucleotides.
Embodiment 252 is the gRNA of any one of embodiments 211-251, wherein the shortened hairpin 1 region has duplex portion 4-8, optionally 7-8 base paired nucleotides in length.
Embodiment 253 is the gRNA of any one of embodiments 211-252, wherein the shortened hairpin 1 region has a single duplex portion.
Embodiment 254 is the gRNA of any one of embodiments 211-253, wherein one or two base pairs of the shortened hairpin 1 region are deleted.
Embodiment 255 is the gRNA of any one of embodiments 211-254, wherein the stem of the shortened hairpin 1 region is seven or eight base paired nucleotides in length.
Embodiment 256 is the gRNA of any one of embodiments 211-255, wherein one or more of positions 85-86 and one or more of nucleotides 91-92 of the shortened hairpin 1 region are deleted.
Embodiment 257 is the gRNA of any one of embodiments 211-256, wherein nucleotides 86 and 91 of the shortened hairpin 1 region are deleted.
Embodiment 258 is the gRNA of any one of embodiments 211-257, wherein one or more of nucleotides 82-95 of the shortened hairpin 1 region is substituted relative to SEQ ID NO: 500.
Embodiment 259 is the gRNA of any one of embodiments 211-258, wherein the second internal linker substitutes for at least part of or for all of nucleotides 87-90.
Embodiment 260 is the gRNA of any one of embodiments 211-259, wherein the second internal linker substitutes for at least part of or for all of nucleotides 81-95.
Embodiment 261 is the gRNA of any one of embodiments 211-260, wherein the shortened hairpin 1 region has 2-15 modified nucleotides.
Embodiment 262 is the gRNA of any one of embodiments 211-261, wherein the shortened hairpin 2 region lacks 2-18, optionally 2-16 nucleotides.
Embodiment 263 is the gRNA of any one of embodiments 211-262, wherein the shortened hairpin 2 region has a length of 22, 23, 24, 25, 26, 27, 28, 29, 30, 31, 32, 33, 34, 35, 36, 37, 38, 39, or 40 nucleotides.
Embodiment 264 is the gRNA of any one of embodiments 211-263, wherein the shortened hairpin 2 region has a length of 24, 25, 26, 27, 28, 29, 30, 31, 32, 33 or 34, nucleotides.
Embodiment 265 is the gRNA of any one of embodiments 211-264, wherein one or more of positions 113-121 and one or more of nucleotides 126-134 of the shortened hairpin 2 region are deleted.
Embodiment 266 is the gRNA of any one of embodiments 211-265, wherein the shortened hairpin 2 region comprises an unpaired region.
Embodiment 267 is the gRNA of any one of embodiments 211-266, wherein the shortened hairpin 2 region has two duplex portions.
Embodiment 268 is the gRNA of embodiment 267, wherein the shortened hairpin 2 region has a duplex portion of 4 base paired nucleotides in length.
Embodiment 269 is the gRNA of embodiments 267-268, wherein the shortened hairpin 2 region has a duplex portion of 4-8 base paired nucleotides in length.
Embodiment 270 is the gRNA of embodiments 267-269, wherein the shortened hairpin 2 region has a duplex portion of 4-6 base paired nucleotides in length.
Embodiment 271 is the gRNA of any one of embodiments 211-270, wherein the upper stem of the shortened hairpin 2 region comprises one, two, three, or four base pairs.
Embodiment 272 is the gRNA of any one of embodiments 211-271, wherein at least one pair of nucleotides 113 and 134, nucleotides 114 and 133, nucleotides 115 and 132, nucleotides 116 and 131, nucleotides 117 and 130, nucleotides 118 and 129, nucleotides 119 and 128, nucleotides 120 and 127, and nucleotides 121 and 126 are deleted.
Embodiment 273 is the gRNA of any one of embodiments 211-272, wherein all of positions 113-121 and 126-134 of the shortened hairpin 2 region are deleted.
Embodiment 274 is the gRNA of any one of embodiments 211-273, wherein one or more of nucleotides 113-134 of the shortened hairpin 2 region is substituted relative to SEQ ID NO: 500.
Embodiment 275 is the gRNA of any one of embodiments 211-274, wherein the third internal linker substitutes for at least part of or for all of nucleotides 122-125.
Embodiment 276 is the gRNA of any one of embodiments 211-275, wherein the third internal linker substitutes for at least part of or for all of nucleotides 112-135.
Embodiment 277 is the gRNA of embodiment any one of embodiments 211-276, wherein the shortened hairpin 2 region has 2-15 modified nucleotides.
Embodiment 278 is the gRNA of any one of embodiments 1-277, wherein the guide region of the gRNA comprises at least two modified nucleotides, optionally at least four modified nucleotides.
Embodiment 279 is the gRNA of any one of embodiments 1-277, wherein the guide region of the gRNA comprises 1, 2, 3, 4, 5, 6, 7, 8, 9, 10, 11, or 12 modified nucleotides, optionally 1, 2, or 3 modified nucleotides.
Embodiment 280 is the gRNA of any one of embodiments 1-279, wherein the guide region of the gRNA comprises 4, 5, 6, 7, 8, 9, 10, 11, or 12 modified nucleotides.
Embodiment 281 is the gRNA of any one of embodiments 1-280, wherein the guide region of the gRNA comprises 6, 7, 8, 9, 10, 11, or 12 modified nucleotides.
Embodiment 282 is the gRNA of any one of embodiments 1-281, wherein the gRNA comprises a 5′ end modification.
Embodiment 283 is the gRNA of any one of embodiments 1-282, wherein the gRNA comprises a 5′ end modification and a 3′ end modification.
Embodiment 284 is the gRNA of any one of embodiments 1-283, wherein the guide region does not comprise a modified nucleotide 3′ of the first three nucleotides of the guide region.
Embodiment 285 is the gRNA of any one of embodiments 211-277, wherein the guide region does not comprise a modified nucleotide.
Embodiment 286 is the gRNA of any one of embodiments 1-285, wherein the gRNA comprises a 3′ end modification.
Embodiment 287 is the gRNA of any one of embodiments 1-286, comprising a modification in the upper stem region of the repeat/anti-repeat region.
Embodiment 288 is the gRNA of any one of embodiments 1-287, comprising a modification in the hairpin 1 region.
Embodiment 289 is the gRNA of any one of embodiments 1-288, comprising a modification in the hairpin 2 region.
Embodiment 290 is the gRNA of any one of embodiments 1-289, comprising a 3′ end modification, and comprising a modification in the upper stem region of the repeat/anti-repeat region.
Embodiment 291 is the gRNA of any one of embodiments 1-290, comprising a 3′ end modification, and a modification in the hairpin 1 region.
Embodiment 292 is the gRNA of any one of embodiments 1-291, comprising a 3′ end modification, and a modification in the hairpin 2 region.
Embodiment 293 is the gRNA of any one of embodiments 1-292, comprising a 5′ end modification, and comprising a modification in the upper stem region of the repeat/anti-repeat region.
Embodiment 294 is the gRNA of any one of embodiments 1-293, comprising a 5′ end modification, and a modification in the hairpin 1 region.
Embodiment 295 is the gRNA of any one of embodiments 1-294, comprising a 5′ end modification, and a modification in the hairpin 2 region.
Embodiment 296 is the gRNA of any one of embodiments 1-295, comprising a 5′ end modification, a modification in the upper stem region of the repeat/anti-repeat region, and a 3′ end modification.
Embodiment 297 is the gRNA of any one of embodiments 1-296, comprising a 5′ end modification, a modification in the hairpin 1 region, and a 3′ end modification.
Embodiment 298 is the gRNA of any one of embodiments 1-297, comprising a 5′ end modification, a modification in the hairpin 1 region, a modification in the hairpin 2 region, and a 3′ end modification.
Embodiment 299 is the gRNA of any one of embodiments 1-298, comprising a 5′ end modification, a modification in the repeat/anti-repeat region, a modification in the hairpin 1 region, a modification in the hairpin 2 region, and a 3′ end modification.
Embodiment 300 is the gRNA of any one of embodiments 282-299, wherein the 5′ end modification comprises a modified nucleotide selected from 2′-O-methyl (2′-OMe) modified nucleotide, 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide, or combinations thereof.
Embodiment 301 is the gRNA of any one of embodiments 283-300, wherein the 3′ end modification comprises a modified nucleotide selected from 2′-O-methyl (2′-OMe) modified nucleotide, 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide, or combinations thereof.
Embodiment 302 is the gRNA of any one of the embodiments 282-301, wherein the 5′ end modification comprises any of:
   i. a modification of any one or more of the first 1, 2, 3, or 4 nucleotides;
   ii. one modified nucleotide;
   iii. two modified nucleotides;
   iv. three modified nucleotides; and
   v. four modified nucleotides.
Embodiment 303 is the gRNA of any one of embodiments 282-302, wherein the 5′ end modification comprises one or more of
   i. a phosphorothioate (PS) linkage between nucleotides;
   ii. a 2′-OMe modified nucleotide;
   iii. a 2′-O-moe modified nucleotide;
   iv. a 2′-F modified nucleotide; and
   v. an inverted abasic modified nucleotide.
Embodiment 304 is the gRNA of any one of embodiments 283-303, wherein the 3′ end modification comprises any of:
   i. a modification of any one or more of the last 4, 3, 2, or 1 nucleotides;
   ii. one modified nucleotide;
   iii. two modified nucleotides;
   iv. three modified nucleotides; and
   v. four modified nucleotides.
Embodiment 305 is the gRNA of any one of embodiments 283-304, wherein the 3′ end modification comprises one or more of
   i. a phosphorothioate (PS) linkage between nucleotides;
   ii. a 2′-OMe modified nucleotide;
   iii. a 2′-O-moe modified nucleotide;
   iv. a 2′-F modified nucleotide; and
   v. an inverted abasic modified nucleotide.
Embodiment 306 is the gRNA of any one of embodiments 282-305, wherein the 5′ end modification comprises at least one PS linkage, and wherein one or more of
   i. there is one PS linkage, and the linkage is between the first and second nucleotides;
   ii. there are two PS linkages between the first three nucleotides;
   iii. there are PS linkages between any one or more of the first four nucleotides; and
   iv. there are PS linkages between any one or more of the first five nucleotides.
Embodiment 307 is the gRNA of embodiment 306, wherein the 5′ end modification further comprises at least one 2′-OMe, 2′-O-moe, inverted abasic, or 2′-F modified nucleotide.
Embodiment 308 is the gRNA of any one of embodiments 282-307, wherein the 5′ end modification comprises:
   i. a modification of one or more of the first 1-4 nucleotides, wherein the modification is a PS linkage, inverted abasic nucleotide, 2′-OMe, 2′-O-moe, 2′-F, or combinations thereof;
   ii. a modification to the first nucleotide with 2′-OMe, 2′-O-moe, 2′-F, or combinations thereof, and an optional one or two PS linkages to the next nucleotide or the first nucleotide of the 3′ tail;
   iii. a modification to the first or second nucleotide with 2′-OMe, 2′-O-moe, 2′-F, or combinations thereof, and optionally one or more PS linkages;
   iv. a modification to the first, second, or third nucleotides with 2′-OMe, 2′-O-moe, 2′-F, or combinations thereof, and optionally one or more PS linkages; or
   v. a modification to the first, second, third or forth nucleotides with 2′-OMe, 2′-O-moe, 2′-F, or combinations thereof, and optionally one or more PS linkages.
Embodiment 309 is the gRNA of any one of embodiments 283-307, wherein the 3′ end modification comprises at least one PS linkage, and wherein one or more of
   i. there is one PS linkage, and the linkage is between the last and second to last nucleotides;
   ii. there are two PS linkages between the last three nucleotides; and
   iii. there are PS linkages between any one or more of the last four nucleotides.
Embodiment 310 is the gRNA of embodiment 309, wherein the 3′ end modification further comprises at least one 2′-OMe, 2′-O-moe, inverted abasic, or 2′-F modified nucleotide.
Embodiment 311 is the gRNA of any one of embodiments 283-310, wherein the 3′ end modification comprises:
   a modification of one or more of the last 1-4 nucleotides, wherein the modification is a PS linkage, inverted abasic nucleotide, 2′-OMe, 2′-O-moe, 2′-F, or combinations thereof;
   a modification to the last nucleotide with 2′-OMe, 2′-O-moe, 2′-F, or combinations thereof, and an optional one or two PS linkages to the next nucleotide or the first nucleotide of the 3′ tail;
   a modification to the last or second to last nucleotide with 2′-OMe, 2′-O-moe, 2′-F, or combinations thereof, and optionally one or more PS linkages;
   a modification to the last, second to last, or third to last nucleotides with 2′-OMe, 2′-O-moe, 2′-F, or combinations thereof, and optionally one or more PS linkages; or
   a modification to the last, second to last, third to last, or fourth to last nucleotides with 2′-OMe, 2′-O-moe, 2′-F, or combinations thereof, and optionally one or more PS linkages.
Embodiment 312 is the gRNA of any one of embodiments 287-311, wherein the modification in the repeat/anti-repeat region, the hairpin 1 region, or the hairpin 2 region comprises a modified nucleotide selected from 2′-O-methyl (2′-OMe) modified nucleotide, 2′-O-(2-methoxyethyl) (2′-O-moe) modified nucleotide, a 2′-fluoro (2′-F) modified nucleotide, a phosphorothioate (PS) linkage between nucleotides, or combinations thereof.
Embodiment 313 is the gRNA of any one of embodiments 287-312, wherein the modification in the repeat/anti-repeat region, the hairpin 1 region, or the hairpin 2 region comprises a modified nucleotide selected from 2′-O-methyl (2′-OMe) modified nucleotide, a 2′-fluoro (2′-F) modified nucleotide, a phosphorothioate (PS) linkage between nucleotides, or combinations thereof.
Embodiment 314 is the gRNA of any one of embodiments 287-313, wherein the modification in the repeat/anti-repeat region, the hairpin 1 region, or the hairpin 2 region comprises a modified nucleotide selected from 2′-O-methyl (2′-OMe) modified nucleotide and a phosphorothioate (PS) linkage between nucleotides, or combinations thereof.
Embodiment 315 is the gRNA of any one of embodiments 1-314, wherein nucleotides 1-3 of the guide region are modified and nucleotides in the guide region other than nucleotides 1-3 are not modified.
Embodiment 316 is the gRNA of any one of embodiments 1-315, wherein a 3′ tail of nucleotide 144 is present in the gRNA, and comprises 2′-O-Me modified nucleotides at nucleotides 141-144 and two PS linkages between nucleotides 141-142 and 142-143 respectively.
Embodiment 317 is a single guide RNA (sgRNA) comprising any one of SEQ ID NOs: 1001-1012 or any other sequences as shown in Table 4A.
Embodiment 318 is the gRNA of any one of embodiments 1-317, comprising a nucleotide sequence having at least 99, 98, 97, 96, 95, 94, 93, 92, 91, 90, 85, 80, 75, or 70% identity to the nucleotide sequence of any one of SEQ ID Nos: 1001-1012 or any other sequences as shown in Table 4A.
Embodiment 319 is the gRNA of any one of embodiments 1-317, comprising a nucleotide sequence having at least 99, 98, 97, 96, 95, 94, 93, 92, 91, 90, 85, 80, 75, or 70% identity to the nucleotide sequence of any one of SEQ ID Nos: SEQ ID Nos: 1001-1002 and 710-759 as shown in Tables 4A-4B, wherein the modification at each nucleotide of the gRNA that corresponds to a nucleotide of the reference sequence identifier in Table 4A is identical to or equivalent to the modification shown in the reference sequence identifier in Table 4B.
Embodiment 320 is the gRNA of any one of embodiments 1-319, comprising a nucleotide sequence having at least 99, 98, 97, 96, 95, 94, 93, 92, 91, or 90% identity to the sequence from X to the 3′ end of the nucleotide sequence of any one of SEQ ID Nos: 1001-1002 and 710-759 as shown in Tables 4A-4B, where X is the first nucleotide of the conserved region.
Embodiment 321 is the gRNA of any one of embodiments 1-230 and 232-320, further comprising a 3′ tail comprising a 2′-O-Me modified nucleotide.
Embodiment 322 is the gRNA of any one of embodiments 1-321, wherein the gRNA directs a nuclease to a target sequence for binding.
Embodiment 323 is the gRNA of any one of embodiments 1-322, wherein the gRNA directs a nuclease to a target sequence for inducing a double-strand break within the target sequence.
Embodiment 324 is the gRNA of any one of embodiments 1-323, wherein the gRNA directs a nuclease to a target sequence for inducing a single-strand break within the target sequence.
Embodiment 325 is the gRNA of any one of embodiments 322-324, wherein the nuclease is a NmeCas9.
Embodiment 326 is the composition of embodiment 325, wherein the Nine Cas9 is an Nme1 Cas9, an Nme2 Cas9, or an Nme3 Cas9.
Embodiment 327 is the gRNA of any one of the preceding embodiments, wherein the gRNA comprising a conservative substitution, e.g., to preserve base pairing.
Embodiment 328 is the gRNA of any one of embodiments 1-327, wherein the internal linker has a bridging length of about 6 Angstroms-37 Angstroms.
Embodiment 329 is the gRNA of any one of embodiments 1-328, wherein the internal linker comprises 1-10 ethylene glycol subunits covalently linked to each other.
Embodiment 330 is the gRNA of any one of embodiments 1-329, wherein the internal linker comprises at least two ethylene glycol subunits covalently linked to each other.
Embodiment 331 is the gRNA of any one of embodiments 1-330, wherein the internal linker comprises 3-10 ethylene glycol subunits covalently linked to each other.
Embodiment 332 is the gRNA of any one of embodiments 1-331, wherein the internal linker comprises 3-6 ethylene glycol subunits covalently linked to each other.
Embodiment 333 is the gRNA of any one of embodiments 1-332, wherein the internal linker comprises 3 ethylene glycol subunits covalently linked to each other.
Embodiment 334 is the gRNA of any one of embodiments 1-333, wherein the internal linker comprises 6 ethylene glycol subunits covalently linked to each other.
Embodiment 335 is the gRNA of any one of embodiments 1-334, wherein the internal linker comprises a structure of formula (I):

˜-L0-L1-L2-# (I)
wherein:
˜ indicates a bond to a 3′ substituent of the preceding nucleotide;
#indicates a bond to a 5′ substituent of the following nucleotide;
L0 is null or C1-3 aliphatic;
L1 is -[E1-(R1)]m-, where
each R1 is independently a C1-5 aliphatic group, optionally substituted with 1 or 2 E2,
each E1 and E2 are independently a hydrogen bond acceptor, or are each
independently chosen from cyclic hydrocarbons, and heterocyclic hydrocarbons, and each m is 1, 2, 3, 4, 5, 6, 7, 8, 9, or 10; and
L2 is null, C1-3 aliphatic, or is a hydrogen bond acceptor.
Embodiment 336 is the gRNA of embodiment 335, wherein m is 6, 7, 8, 9, or 10.
Embodiment 337 is the gRNA of any one of embodiments 335-336, wherein m is 1, 2, 3, 4 or 5.
Embodiment 338 is the gRNA of any one of embodiments 335-337, wherein m is 1, 2, or 3.
Embodiment 339 is the gRNA of any one of embodiments 335-338, wherein L0 is null.
Embodiment 340 is the gRNA of any one of embodiments 335-338, wherein L0 is —CH2- or —CH2CH2-.
Embodiment 341 is the gRNA of any one of embodiments 335-340, wherein L2 is null.
Embodiment 342 is the gRNA of any one of embodiments 335-340, wherein L2 is —O—, —S—, —CH2- or —CH2CH2-.
Embodiment 343 is the gRNA of any one of embodiments 335-342, wherein the number of atoms in the shortest chain of atoms on the pathway from ˜ to #in the structure of Formula (I) is 30 or less, 27 or less, 24 or less, 21 or less, or is 18 or less, or is 15 or less, or is 12 or less, or is 10 or less.
Embodiment 344 is the gRNA of any one of embodiments 335-343, wherein the number of atoms in the shortest chain of atoms on the pathway from ˜ to #in the structure of Formula (I) is from 6 to 30, optionally 9 to 30, optionally 9 to 21.
Embodiment 345 is the gRNA of any one of embodiments 335-344, wherein the number of atoms in the shortest chain of atoms on the pathway from ˜ to #in the structure of Formula (I) is 9.
Embodiment 346 is the gRNA of any one of embodiments 335-344, wherein the number of atoms in the shortest chain of atoms on the pathway from ˜ to #in the structure of Formula (I) is 18.
Embodiment 347 is the gRNA of any one of embodiments 335-346, wherein each C1-3 aliphatic group and C1-5 aliphatic group is saturated.
Embodiment 348 is the gRNA of any one of embodiments 335-346, wherein at least one C1-5 aliphatic group is a C1-4 alkylene, or wherein at least two C1-5 aliphatic groups are a C1-4 alkylene, or wherein at least three C1-5 aliphatic groups are a C1-4 alkylene.
Embodiment 349 is the gRNA of any one of embodiments 335-348, wherein at least one R1 is selected from —CH2-, —CH2CH2-, —CH2CH2CH2-, or —CH2CH2CH2CH2-.
Embodiment 350 is the gRNA of any one of embodiments 335-348, wherein each R1 is independently selected from —CH2-, —CH2CH2-, —CH2CH2CH2-, or —CH2CH2CH2CH2-.
Embodiment 351 is the gRNA of any one of embodiments 335-350, wherein each R1 is —CH2CH2-.
Embodiment 352 is the gRNA of any one of embodiments 335-351, wherein at least one C1-5 aliphatic group is a C1-4 alkenylene, or wherein at least two C1-5 aliphatic groups are a C1-4 alkenylene, or wherein at least three C1-5 aliphatic groups are a C1-4 alkenylene.
Embodiment 353 is the gRNA of any one of embodiments 335-352, wherein at least one R1 is selected from —CHCH—, —CHCHCH2-, or —CH2CHCHCH2-.
Embodiment 354 is the gRNA of any one of embodiments 335-353, wherein each E1 is independently chosen from —O—, —S—, —NH—, —NR—, —C(O)—O—, —OC(O)O—, —C(O)—NR—, —OC(O)—NR—, —NC(O)—NR—, —P(O)2O—, —OP(O)2O—, —OP(R)(O)O—, —OP(O)(S)O—, —S(O)2-, cyclic hydrocarbons, and heterocyclic hydrocarbons.
Embodiment 355 is the gRNA of any one of embodiments 335-354, wherein each E1 is independently chosen from —O—, —S—, —NH—, —NR—, —C(O)—O—, —OC(O)O—, —P(O)2O—, —OP(O)2O—, and —OP(R)(O)O.
Embodiment 356 is the gRNA of any one of embodiments 335-355, wherein each E1 is —O—.
Embodiment 357 is the gRNA of any one of embodiments 335-355, wherein each E1 is —S—.
Embodiment 358 is the gRNA of any one of embodiments 335-357, wherein at least one C1-5 aliphatic group in R1 is optionally substituted with one E2.
Embodiment 359 is the gRNA of any one of embodiments 335-358, wherein each E2 is independently chosen from —OH, —OR, —ROR, —SH, —SR, —C(O)—R, —C(O)—OR, —OC(O)—OR, —C(O)—H, —C(O)—OH, —OPO3, —PO3, —RPO3, —S(O)2-R, —S(O)2-OR, —RS(O)2-R, —RS(O)2-OR, —SO3, cyclic hydrocarbons, and heterocyclic hydrocarbons.
Embodiment 360 is the gRNA of any one of embodiments 335-359, wherein each E2 is independently chosen from —OH, —OR, —SH, —SR, —C(O)—R, —C(O)—OR, —OC(O)—OR, —OPO3, —PO3, —RPO3, and —SO3.
Embodiment 361 is the gRNA of any one of embodiments 335-360, wherein each E2 is —OH or —OR.
Embodiment 362 is the gRNA of any one of embodiments 335-360, wherein each E2 is —SH or —SR.
Embodiment 363 is the gRNA of any one of embodiments 335-362, wherein the internal linker comprises a PEG-linker.
Embodiment 364 is the gRNA of any one of embodiments 335-363, wherein the internal linker comprises a PEG-linker having from 1 to 10 ethylene glycol units.
Embodiment 365 is the gRNA of any one of embodiments 335-364, wherein the internal linker comprises a PEG-linker having from 3 to 6 ethylene glycol units.
Embodiment 366 is the gRNA of any one of embodiments 335-365, wherein the internal linker comprises a PEG-linker having 3 ethylene glycol units.
Embodiment 367 is the gRNA of any one of embodiments 335-365, wherein the internal linker comprises a PEG-linker having 6 ethylene glycol units.
Embodiment 368 is the gRNA of any one of embodiments 1-367, wherein the gRNA is a short guide RNA comprising a shortened conserved portion, and the internal linker substitutes for at least 2, 3, 4, 5, 6, 7, 8, 9, 10, 11, or 12 nucleotides.
Embodiment 369 is the gRNA of any one of embodiments 1-210 or 278-368, wherein the gRNA is a short-single guide RNA (short-sgRNA) comprising a conserved portion of an sgRNA comprising a hairpin region, wherein the hairpin region lacks at least 5-10 nucleotides.
Embodiment 370 is the gRNA of embodiment 369, wherein the at least 5-10 lacking nucleotides are consecutive.
Embodiment 371 is the gRNA of any one of embodiments 369-370, wherein the at least 5-10 lacking nucleotides
   i. are within hairpin 1;
   ii. are within hairpin 1 and the “N” between hairpin 1 and hairpin 2 relative to SEQ ID NO: 400;
   iii. are within hairpin 1 and the two nucleotides immediately 3′ of hairpin 1;
   iv. include at least a portion of hairpin 1;
   v. are within hairpin 2;
   vi. include at least a portion of hairpin 2;
   vii. are within hairpin 1 and hairpin 2;
   viii. include at least a portion of hairpin 1 and include the “N” between hairpin 1 and hairpin 2 relative to SEQ ID NO: 400;
   ix. include at least a portion of hairpin 2 and include the “N” between hairpin 1 and hairpin 2 relative to SEQ ID NO: 400;
   x. include at least a portion of hairpin 1, include the “N” between hairpin 1 and hairpin 2 relative to SEQ ID NO: 400, and include at least a portion of hairpin 2;
   xi. are within hairpin 1 or hairpin 2, optionally including the “N” between hairpin 1 and hairpin 2 relative to SEQ ID NO: 400;
   xii. are consecutive;
   xiii. are consecutive and include the “N” between hairpin 1 and hairpin 2 relative to SEQ ID NO: 400;
   xiv. are consecutive and span at least a portion of hairpin 1 and a portion of hairpin 2;
   xv. are consecutive and span at least a portion of hairpin 1 and the “N” between hairpin 1 and hairpin 2 relative to SEQ ID NO: 400; or
   xvi. are consecutive and span at least a portion of hairpin 1 and two nucleotides immediately 3′ of hairpin 1.
Embodiment 372 is the gRNA of any one of embodiments 1-210 or 278-371, wherein the gRNA is a short-single guide RNA (short-sgRNA) comprising a conserved portion of an sgRNA comprising a hairpin region, wherein the hairpin region lacks at least 5-10 nucleotides and wherein the short-sgRNA comprises a 5′ end modification or a 3′ end modification.
Embodiment 373 is the gRNA of any one of embodiments 1-210 or 278-372, wherein the at least 5-10 nucleotides comprise nucleotides 54-61 of SEQ ID NO:400, nucleotides 53-60 of SEQ ID NO:400; or nucleotides 54-58 of SEQ ID NO:400, optionally wherein the short-sgRNA comprises modifications at least H1-1 to H1-5 and H2-1 to H2-12.
Embodiment 374 is the gRNA of any one of embodiments 1-210 or 278-373, comprising a shortened hairpin 1 region or a substituted and optionally shortened hairpin 1 region, wherein
   (i) at least one of the following pairs of nucleotides are substituted in the substituted and optionally shortened hairpin 1 with Watson-Crick pairing nucleotides: H1-1 and H1-12, H1-2 and H1-11, H1-3 and H1-10, or H1-4 and H1-9, and the hairpin 1 region optionally lacks
      (aa) any one or two of H1-5 through H1-8,
      (bb) one, two, or three of the following pairs of nucleotides: H1-1 and H1-12, H1-2 and H1-11, H1-3 and H1-10 or H1-4 and H1-9, or
      (cc) 1-8 nucleotides of the hairpin 1 region; or
   (ii) the shortened hairpin 1 region lacks 6-8 nucleotides, preferably 6 nucleotides; and
      (aa) one or more of positions H1-1, H1-2, or H1-3 is deleted or substituted relative to SEQ ID NO: 400 or
      (bb) one or more of positions H1-6 through H1-10 is substituted relative to SEQ ID NO: 400; or
   (iii) the shortened hairpin 1 region lacks 5-10 nucleotides, preferably 5-6 nucleotides, and one or more of positions N18, H1-12, or n is substituted relative to SEQ ID NO: 400.
Embodiment 375 is the gRNA of any one of embodiments 1-210 or 278-374, comprising a shortened upper stem region, wherein the shortened upper stem region lacks 1-6 nucleotides and wherein the 6, 7, 8, 9, 10, or 11 nucleotides of the shortened upper stem region include less than or equal to 4 substitutions relative to SEQ ID NO: 400.
Embodiment 376 is the gRNA of any one of embodiments 1-210 or 278-375, comprising a substitution relative to SEQ ID NO: 400 at any one or more of LS6, LS7, US3, US10, B3, N7, N15, N17, H2-2 and H2-14, wherein the substituent nucleotide is neither a pyrimidine that is followed by an adenine, nor an adenine that is preceded by a pyrimidine.
Embodiment 377 is the gRNA of embodiment 374, wherein the shortened and substituted hairpin 1 lacks 1-4 nucleotides and nucleotides H1-4 through H1-9 are substituted by an internal linker.
Embodiment 378 is the gRNA of embodiment 374, wherein the shortened and substituted hairpin 1 lacks one or two of the following pairs of nucleotides: H1-1 and H1-12, H1-2 and H1-11, or H1-3 and H1-10; and nucleotides H1-4 through H1-9 are substituted by an internal linker.
Embodiment 379 is the gRNA of any one of embodiments 1-210 or 278-378, comprising an upper stem region, wherein the upper stem modification comprises a modification to any one or more of US1-US12 in the upper stem region.
Embodiment 380 is the gRNA of any one of embodiments 1-210 or 278-379, comprising a shortened upper stem region, wherein the shortened upper stem region lacks 1-6 nucleotides.
Embodiment 381 is the gRNA of any one of embodiments 1-210 or 278-379, comprising a shortened upper stem region, wherein the shortened upper stem region lacks 7-10 nucleotides and 2 nucleotides are substituted by an internal linker.
Embodiment 382 is the gRNA of embodiment 381, wherein the stem does not comprise an upper stem duplex portion.
Embodiment 383 is the gRNA of embodiment 381 or 382 wherein the internal linker has a bridging length of about 3-30 atoms, optionally 12-21 atoms, 6-18 atoms, or 6-12 atoms.
Embodiment 384 is the gRNA of any one of the preceding embodiments, wherein the gRNA comprises a modification.
Embodiment 385 is the guide RNA of embodiment 384, wherein the modification comprises a 2′-O-methyl (2′-O-Me) modified nucleotide, a 2′-F modified nucleotide, 2′-H modified nucleotide (DNA), a 2′-O,4′-C-ethylene modified nucleotides (ENA), locked nucleotide (LNA), or unlocked nucleotide (UNA).
Embodiment 386 is the guide RNA of embodiment 384 or 385, wherein the modification comprises a phosphorothioate (PS) bond between nucleotides.
Embodiment 387 is the guide RNA of any one of embodiments 384-386, wherein the guide RNA is a sgRNA and the modification, comprises a modification at one or more of the five nucleotides at the 5′ end of the guide RNA.
Embodiment 388 is the guide RNA of any one of embodiments 384-387, wherein the guide RNA is a sgRNA and the modification, comprises a modification at one or more of the five nucleotides at the 3′ end of the guide RNA.
Embodiment 389 is the guide RNA of any one of embodiments 384-388, wherein the guide RNA is a sgRNA and the modification, comprises a PS bond between each of the four nucleotides at the 5′ end of the guide RNA.
Embodiment 390 is the guide RNA of any one of embodiments 384-389, wherein the guide RNA is a sgRNA and the modification, comprises a PS bond between each of the four nucleotides at the 3′ end of the guide RNA.
Embodiment 391 is the guide RNA of any one of embodiments 384-389, wherein the guide RNA is a sgRNA and the modification, comprises a 2′-O-Me modified nucleotide at each of the first three nucleotides at the 5′ end of the guide RNA.
Embodiment 392 is the guide RNA of any one of embodiments 384-390, wherein the guide RNA is a sgRNA and the modification, comprises a 2′-O-Me modified nucleotide at each of the last three nucleotides at the 3′ end of the guide RNA.
Embodiment 393 is the gRNA of any one of the preceding embodiments, wherein the 3′ nucleotide of the gRNA is a nucleotide with a uracil base.
Embodiment 394 is the gRNA of any one of the preceding embodiments, wherein the gRNA comprises a 3′ tail.
Embodiment 395 is the gRNA of embodiment 394, wherein the 3′ tail comprises at least 1-10 nucleotides.
Embodiment 396 is the gRNA of any one of embodiments 394-395, wherein the 3′ tail terminates with a nucleotide with a uracil base.
Embodiment 397 is the gRNA of any one of embodiments 394-396, wherein the 3′ tail is 1 nucleotide in length and is a nucleotide with a uracil base.
Embodiment 398 is the gRNA of any one of embodiments 394-397, wherein the 3′ tail comprises a modification of any one or more of the nucleotides present in the 3′ tail.
Embodiment 399 is the gRNA of embodiment 393-398, wherein the 3′ tail is fully modified.
Embodiment 400 is the gRNA of any one of embodiments 1-393, wherein the gRNA does not comprise a 3′ tail.
Embodiment 401 is the gRNA of any one of the preceding embodiments, wherein the gRNA comprises a 3′ end modification or a 5′ end modification.
Embodiment 402 is the gRNA of any one of the preceding embodiments, wherein the gRNA comprises a 5′ end modification and a 3′ end modification.
Embodiment 403 is the gRNA of any one of embodiments 401-402, wherein the 3′ or 5′ end modification comprises a protective end modification, optionally a modified nucleotide selected from a 2′-O-methyl (2′-OMe) modified nucleotide, a 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide, or a combination thereof.
Embodiment 404 is the gRNA of any one of embodiments 401-403, wherein the 3′ or 5′ end modification comprises or further comprises a 2′-O-methyl (2′-Ome) modified nucleotide.
Embodiment 405 is the gRNA of any one of embodiments 401-404, wherein the 3′ or 5′ end modification comprises or further comprises a 2′-fluoro (2′-F) modified nucleotide.
Embodiment 406 is the gRNA of any one of embodiments 401-405, wherein the 3′ or 5′ end modification comprises or further comprises a phosphorothioate (PS) linkage between nucleotides.
Embodiment 407 is the gRNA of any one of embodiments 401-406, wherein the 3′ or 5′ end modification comprises or further comprises an inverted abasic modified nucleotide.
Embodiment 408 is the gRNA of any one of the preceding embodiments, comprising a modification in a or the hairpin region.
Embodiment 409 is the gRNA of embodiment 408, comprising a modification in the hairpin region, wherein the modification in the hairpin region comprises a modified nucleotide selected from a 2′-O-methyl (2′-Ome) modified nucleotide, a 2′-fluoro (2′-F) modified nucleotide, a phosphorothioate (PS) linkage between nucleotides, or a combination thereof.
Embodiment 410 is the gRNA of embodiment 408 or 409, further comprising a 3′ end modification.
Embodiment 411 is the gRNA of embodiment 408 or 409, further comprising a 3′ end modification and a 5′ end modification.
Embodiment 412 is the gRNA of embodiment 408 or 409, further comprising a 5′ end modification.
Embodiment 413 is the gRNA of any one of embodiments 408-412, wherein the modification in the hairpin region comprises or further comprises a 2′-O-methyl (2′-Ome) modified nucleotide.
Embodiment 414 is the gRNA of any one of embodiments 408-413, wherein the modification in the hairpin region comprises or further comprises a 2′-fluoro (2′-F) modified nucleotide.
Embodiment 415 is the gRNA of any one of the preceding embodiments, comprising a modification in a or the upper stem region.
Embodiment 416 is the gRNA of embodiment 415, wherein the upper stem modification comprises any one or more of:
   i. a modification of any one or more of US1-US12 in the upper stem region (corresponding to nucleotides 9-20 of SEQ ID NO: 400); and
   ii. a modification of at least 1, 2, 3, 4, 5, 6, 7, 8, 9, 10, 11, or all 12 nucleotides in the upper stem region.
Embodiment 417 is the gRNA of embodiment 415 or 416, wherein the upper stem modification comprises one or more of:
   i. a 2′-OMe modified nucleotide;
   ii. a 2′-O-moe modified nucleotide;
   iii. a 2′-F modified nucleotide;
   iv. 2′-H modified nucleotide (DNA);
   v. a 2′-O,4′-C-ethylene modified nucleotides (ENA);
   vi. locked nucleotide (LNA);
   vii. unlocked nucleotide (UNA); and
   viii. combinations of one or more of (i.)-(vii.).
Embodiment 418 is the gRNA of any one of the preceding embodiments, wherein the modification comprises a YA modification.
Embodiment 419 is the gRNA of any one of the preceding embodiments, comprising a YA modification of one or more guide region YA sites.
Embodiment 420 is the gRNA of any one of embodiments 418-419, wherein the YA modification comprises a substitution of the pyrimidine of a YA site with a non-pyrimidine.
Embodiment 421 is the gRNA of any one of embodiments 418-419, wherein the YA modification comprises a substitution of the adenine of a YA site with a non-adenine.
Embodiment 422 is the gRNA of any one of embodiments 418-421, comprising a YA modification wherein the modification comprises 2′-fluoro, 2′-H, 2′-OMe, ENA, UNA, inosine, or PS modification.
Embodiment 423 is the gRNA of any one of the preceding embodiments, comprising a YA modification of one or more conserved region YA sites.
Embodiment 424 is the gRNA of any one of the preceding embodiments, wherein the YA modification comprises
   (i) a 2′-OMe modification, optionally of the pyrimidine of the YA site;
   (ii) a 2′-fluoro modification, optionally of the pyrimidine of the YA site; or
   (iii) a PS modification, optionally of the pyrimidine of the YA site.
Embodiment 425 is the gRNA of any one of embodiments 61-210 or 278-424, comprising a nucleotide sequence having at least 99, 98, 97, 96, 95, 94, 93, 92, 91, 90, 85, 80, 75, or 70% identity to the nucleotide sequence of any one of SEQ ID NOs: 1-8, 20-75, 77-84, 101-108, 120-175, and 177-184.
Embodiment 426 is the gRNA of any one of embodiments 61-210 or 278-425, comprising a nucleotide sequence having at least 99, 98, 97, 96, 95, 94, 93, 92, 91, 90, 85, 80, 75, or 70% identity to the nucleotide sequence of any one of SEQ ID Nos: 1-8, 20-75, 77-92, 101-108, and 120-175, and 177-184, wherein the modification at each nucleotide of the gRNA that corresponds to a nucleotide of the reference sequence identifier in Table 2A is identical to or equivalent to the modification shown in the reference sequence identifier in Table 2B.
Embodiment 427 is a guide RNA (gRNA) comprising any of SEQ ID NOs: 1-8, and 20-75, and 77-84.
Embodiment 428 is the gRNA of any one of the preceding embodiments, including modifications set forth for a guide RNA in Table 2A or Table 2B.
Embodiment 429 is a guide RNA (gRNA) comprising any one of SEQ ID NOs: 101-108, and 120-175, and 177-184, including the modifications of Table 2A or Table 2B.
Embodiment 430 is a single guide RNA (sgRNA) comprising any one of SEQ ID NOs: 211-230 or any other sequences as shown in Tables 2A-2C.
Embodiment 431 is the gRNA of any one of the preceding embodiments, comprising a nucleotide sequence having at least 99, 98, 97, 96, 95, 94, 93, 92, 91, 90, 85, 80, 75, or 70% identity to the nucleotide sequence of any one of SEQ ID Nos: 211-230 or any other sequences as shown in Tables 2A-2C.
Embodiment 432 is the gRNA of any one of the preceding embodiments, comprising a nucleotide sequence having at least 99, 98, 97, 96, 95, 94, 93, 92, 91, 90, 85, 80, 75, or 70% identity to the nucleotide sequence of any one of SEQ ID NOs: 101-108, 120-175, 177-184, 211-230 as shown in Tables 2A-2C, wherein the modification at each nucleotide of the gRNA that corresponds to a nucleotide of the reference sequence identifier in Table 2C is identical to or equivalent to the modification shown in the reference sequence identifier in Table 2A or 2B.
Embodiment 433 is the gRNA of any one of the preceding embodiments, comprising a nucleotide sequence having at least 99, 98, 97, 96, 95, 94, 93, 92, 91, or 90% identity to the sequence from X to the 3′ end of the nucleotide sequence of any one of SEQ ID NOs: 101-108, 120-175, 177-184, and 211-230 as shown in Tables 2A-2C, where X is the first nucleotide of the conserved region.
Embodiment 434 is the gRNA of any one of the preceding embodiments, wherein the gRNA is associated with a lipid nanoparticle (LNP).
Embodiment 435 is a composition comprising the gRNA of any one of the preceding embodiments.
Embodiment 436 is a composition comprising a gRNA of any one of embodiments 1-434 associated with a lipid nanoparticle (LNP).
Embodiment 437 is a composition comprising the gRNA of any one of embodiments 1-434, or the composition of any one of embodiment 435 or 436, further comprising a nuclease or an mRNA which encodes the nuclease.
Embodiment 438 is an LNP composition comprising a gRNA of any one of embodiments 1-434.
Embodiment 439 is an LNP composition comprising a gRNA of any one of embodiments 63-116 and 278-433 and an mRNA encoding SpyCas9.
Embodiment 440 is an LNP composition comprising a gRNA of any one of embodiments 117-159 and 278-433 and an mRNA encoding SauCas9.
Embodiment 441 is a LNP composition comprising a gRNA of any one of embodiments 160-189 and 278-433 and an mRNA encoding St1Cas9.
Embodiment 442 is a LNP composition comprising a gRNA of any one of embodiments 190-202 and 278-433 and an mRNA encoding CjeCas9 or FnoCas9.
Embodiment 443 is a LNP composition comprising a gRNA of any one of embodiments 203-210 and 278-433 and an mRNA encoding AsCpf1, LbCpf1, or EsCas13d.
Embodiment 444 is the LNP composition of any one of embodiments 438-443, wherein the LNP comprises (9z,12z)-3-((4,4-bis(octyloxy)butanoyl)oxy)-2-((((3-(diethylamino)propoxy)carbonyl)oxy)methyl)propyl octadeca-9,12-dienoate or nonyl 8-((7,7-bis(octyloxy)heptyl)(2-hydroxyethyl)amino)octanoate.
Embodiment 445 is the composition of any one of embodiments 438-444, wherein the LNP comprises a molar ratio of a cationic lipid amine to RNA phosphate (N:P) of about 4.5-6.5, optionally the N:P of about 6.0.
Embodiment 446 is the composition of embodiment 438-445, wherein the nuclease comprises a protein or a nucleic acid encoding the nuclease.
Embodiment 447 is the composition of embodiment 446, wherein the nuclease is a Cas nuclease.
Embodiment 448 is the composition of embodiment 447, wherein the Cas nuclease is a Cas9.
Embodiment 449 is the composition of embodiment 448, wherein the Cas9 is S. pyogenes Cas9 (SpyCas9).
Embodiment 450 is the composition of embodiment 448, wherein the Cas9 is S. aureus Cas9 (SauCas9).
Embodiment 451 is the composition of embodiment 448, wherein the Cas9 is C. diphtheriae Cas9 (CdiCas9).
Embodiment 452 is the composition of embodiment 448, wherein the Cas9 is Streptococcus thermophilus Cas9 (St1Cas9).
Embodiment 453 is the composition of embodiment 448, wherein the Cas9 is A. cellulolyticus Cas9 (AceCas9).
Embodiment 454 is the composition of embodiment 448, wherein the Cas9 is C. jejuni Cas9 (CjeCas9).
Embodiment 455 is the composition of embodiment 448, wherein the Cas9 is R. palustris Cas9 (RpaCas9).
Embodiment 456 is the composition of embodiment 448, wherein the Cas9 is R. rubrum Cas9 (RruCas9).
Embodiment 457 is the composition of embodiment 448, wherein the Cas9 is A. naeslundii Cas9 (AnaCas9).
Embodiment 458 is the composition of embodiment 448, wherein the Cas9 is Francisella novicida Cas9 (FnoCas9).
Embodiment 459 is the composition of embodiment 448, wherein the Cas nuclease is a Cpf1.
Embodiment 460 is the composition of embodiment 459, wherein the Cpf1 is Lachnospiraceae bacterium Cpf1 (LbCpf1) or the Cpf1 is Acidaminococcus sp. Cpf1 (AsCpf1).
Embodiment 461 is the composition of embodiment 448, wherein the Cas protein is an Eubacterium siraeum Cas13d (EsCas13d).
Embodiment 462 is the composition of embodiment 448, wherein the Cas9 is a Nme Cas9.
Embodiment 463 is the composition of embodiment 462, wherein the Cas9 is an Nme1 Cas9, an Nme2 Cas9, or an Nme3 Cas9.
Embodiment 464 is the composition of any one of embodiments 439-463, wherein the nuclease is a cleavase, a nickase, or a catalytically inactive nuclease, or is a fusion protein comprising a deaminase.
Embodiment 465 is the composition of any one of embodiments 439-464, wherein the nuclease is modified.
Embodiment 466 is the composition of the immediately preceding embodiment, wherein the modified nuclease comprises a nuclear localization signal (NLS).
Embodiment 467 is the composition of embodiment 439-466, wherein the nucleic acid encoding the nuclease is selected from:
   a. a DNA coding sequence;
   b. an mRNA with an open reading frame (ORF);
   c. a coding sequence in an expression vector;
   d. a coding sequence in a viral vector.
Embodiment 468 is the composition of the immediately preceding embodiment, wherein the mRNA comprises the sequence of any one of SEQ ID NOs: 321-323, 361, 363-372, and 374-382.
Embodiment 469 is a pharmaceutical formulation comprising the gRNA of any one of embodiments 1-434, or the composition of any one of embodiments 435-468 and a pharmaceutically acceptable carrier.
Embodiment 470 is a method of modifying a target DNA comprising, delivering to a cell any one or more of the following: i. the gRNA of any one of embodiments 1-434; ii. the composition of any one of embodiments 435-468; and iii. the pharmaceutical formulation of embodiment 469.
Embodiment 471 is the method of embodiment 470, wherein the gRNA comprises no more than 110, 115, 110, 105, 100, 95, 90, 85, 80, 75, 70, 65, 60, 55, 50, 45, or 40, nucleotides.
Embodiment 472 is the method of embodiment 470 or 471, wherein the method results in an insertion or deletion in a gene.
Embodiment 473 is the method of embodiment 472, wherein the method results in an insertion or deletion in a base edit.
Embodiment 474 is the method of any one of embodiments 470-473, further comprising delivering to the cell a template, wherein at least a part of the template incorporates into a target DNA at or near a double strand break site induced by the Cas protein.
Embodiment 475 is the gRNA of any one of embodiments 1-434, the composition of embodiments 435-468, or the pharmaceutical formulation of embodiment 469 for use in preparing a medicament for treating a disease or disorder.
Embodiment 476 is use of the gRNA of any one of embodiments 1-427, the composition of embodiments 435-468, or the pharmaceutical formulation of embodiment 460 469 in the manufacture of a medicament for treating a disease or disorder.
Embodiment 477 is a chemically synthesized gRNA comprising an internal linker.
Embodiment 478 is a composition comprising the gRNA of any one of embodiments 1-434, wherein the composition does not comprise an unlinked portion of the gRNA.
Embodiment 479 is a solid support covalently attached to the linker of the gRNA of any one of embodiments 1-434.
Embodiment 480 is a method of synthesizing a gRNA comprising an internal linker wherein it is a single synthetic process.
Embodiment 481 is a method of synthesizing a gRNA wherein an internal linker is incorporated in line during synthesis.
Embodiment 482 is a method of synthesizing a gRNA using a series of sequential coupling reactions wherein the reactions comprise:
   a) coupling reaction for covalent linkage of a first nucleotide to a second nucleotide;
   b) a coupling reaction for covalent linkage of an internal linker to the second nucleotide; and
   c) a coupling reaction for covalent linkage of a third nucleotide to the internal linker, wherein the coupling reaction for the covalent linkages are all the same.
Embodiment 483 is the method of embodiment 482, wherein covalent linkage is performed using phosphoramidite chemistry.
Embodiment 484 is the gRNA, composition, formulation, method, or use of any one of the preceding embodiments, wherein the gRNA is chemically synthesized.
Embodiment 485 is the gRNA, composition, formulation, method, or use of any one of the preceding embodiments, wherein the internal linker is incorporated into the gRNA via a coupling reaction during chemical synthesis of the gRNA.
Embodiment 486 is the gRNA, composition, formulation, method, or use of any one of the preceding embodiments, prepared by a process comprising addition of the internal linker by reacting a linker comprising a phosphoramidite moiety with a nucleoside residue.
Embodiment 487 is the gRNA, composition, formulation, method, or use of the immediately preceding embodiment, wherein the process further comprises reacting a nucleotide comprising a phosphoramidite moiety with the linker.
Embodiment 488 is the gRNA, composition, formulation, method, or use of any one of the preceding embodiments, wherein the internal linker is covalently joined to the adjacent nucleotide by a phosphodiester or a phosphorothioate bond.
Embodiment 489 is the gRNA, composition, formulation, method, or use of any one of the preceding embodiments, wherein no urea is present in the internal linker.
Embodiment 490 is the gRNA, composition, formulation, method, or use of any one of the preceding embodiments, wherein the internal linker is not in the repeat-anti-repeat region of the gRNA.
Embodiment 491 is the gRNA, composition, formulation, method, or use of any one of the preceding embodiments, wherein the gRNA comprises an internal linker that is not in a repeat-anti-repeat of the guide.
Embodiment 492 is the gRNA, composition, formulation, method, or use of any one of the preceding embodiments, wherein the gRNA is an sgRNA.
Embodiment 493 is the gRNA, composition, formulation, method, or use of any one of the preceding embodiments, wherein the internal linker bridges a duplex region and substitutes for 2-12 nucleotides.
Embodiment 494 is the gRNA, composition, formulation, method, or use of any one of the preceding embodiments, wherein the gRNA is made in a single synthesis.

### FIGURE LEGENDS

FIGS. 1A-1C show the % editing of the indicated guides with internal linkers delivered in vitro using lipofection in (A) primary mouse hepatocytes (PMH), (B) primary cynomolgus hepatocytes (PCH), and (C) primary human hepatocytes (PHH).

FIGS. 2A and 2B show dose response curves for % editing results for (A) set 1 and (B) set 2 from experiments in which guides with internal linkers were delivered in vitro PCH using lipofection.

FIGS. 3A and 3B show dose response curves for % editing results from experiments in which guides with internal linkers were delivered in vitro to (A) PMH and (B) PCH using lipofection.

FIGS. 4A-4C show dose response curves for % editing results from experiments in which guides with internal linkers were delivered in vitro to (A) PMH, (B) PCH, and (C) PRH using lipofection.

FIGS. 5A and 5B show results from in vivo mouse studies providing (A) % editing and (B) serum TTR concentration (ug/ml) for the indicated guides with internal linkers administered at a dose of 0.1 mg/kg of total RNA.

FIG. 6 show results from in vivo mouse studies providing % editing for the indicated guides with internal linkers administered at a dose of 0.1 mg/kg or 0.03 mg/kg of total RNA.

FIG. 7 show results from in vivo mouse studies providing % editing for the indicated guides with internal linkers administered at a dose of 0.1 mg/kg or 0.03 mg/kg of total RNA.

FIGS. 8A and 8B show results from in vivo rat studies providing (A) % editing and (B) serum TTR concentration (ug/ml) for the indicated guides with internal linkers administered at a dose of 0.1 mg/kg or 0.03 mg/kg of total RNA.

FIG. 9 shows a representation of various Spy Cas9 guides with internal linkers paired with results from studies presented in prior figures.

FIGS. 10A-10E show exemplary guide structures (linkers not shown) for (A) Spy Cas9, (B) Sau Cas9, (C) AsCas12A (AsCpf1), (D) EsCas 13D, and (E) NmeCas9, indicating the targeting region (gray fill with dashed outline, not amenable to internal linker substitution), bases not amenable to internal linker substitution (gray fill with solid outline), bases amenable to single or pairwise deletion (open circles), bases amenable to substitution with a long linker (checked fill with solid outline), and bases amenable to substitution with a short linker (crosshatch fill with solid outline).

FIG. 11 shows an exemplary sgRNA (SEQ ID NO: 300, methylation not shown) in a possible secondary structure with labels designating individual nucleotides of the conserved region of the sgRNA, including the lower stem, bulge, upper stem, nexus (the nucleotides of which can be referred to as N1 through N18, respectively, in the 5′ to 3′ direction), and the hairpin region which includes hairpin 1 and hairpin 2 regions. A nucleotide between hairpin 1 and hairpin 2 is labeled n. A guide region may be present on an sgRNA and is indicated in this figure as “(N)ₓ” preceding the conserved region of the sgRNA.

FIG. 12A shows mean percent editing at the TTR locus in PMH using varying ratios of sgRNA and Nme2Cas9 mRNA.

FIG. 12B shows mean percent editing at the TTR locus in PMH using varying ratios a pgRNA and Nme2Cas9 mRNA.

FIG. 13 shows mean percent editing at the TTR locus in PMH for pgRNAs with Nme2Cas9 mRNA.

FIG. 14A shows mean percent editing at TTR exon 1 in PMH for pgRNAs with 2′-OMe modification in the guide sequence with Nme2Cas9 mRNA.

FIG. 14B shows mean percent editing at TTR exon 3 in PMH for pgRNAs with 2′-OMe modification in the guide sequence with Nme2Cas9 mRNA.

FIG. 14C shows mean percent editing at TTR exon 1 in PMH for pgRNAs with light 2′-OMe modification in the guide sequence with Nme2Cas9 mRNA.

FIG. 14D shows mean percent editing at TTR exon 3 in PMH for pgRNAs with light 2′-OMe modification in the guide sequence with Nme2Cas9 mRNA.

FIG. 15 shows mean percent editing at the mouse TTR locus in PMH cells treated with NmeCas9 constructs designed with 1 or 2 nuclear localization sequences.

FIG. 16 shows mean percent editing at the mouse TTR locus in PMH cells treated with pgRNA and various Nme2Cas9 mRNAs.

FIG. 17A shows mean percent editing at the TTR locus in mouse liver following treatment with pgRNA and Nme2Cas9.

FIG. 17B shows mean serum TTR protein following treatment with pgRNA and Nme2Cas9.

FIG. 17C shows mean percent TTR knockdown following treatment with pgRNA and Nme2Cas9.

FIG. 17D shows mean percent editing at the TTR locus in mouse liver following treatment with pgRNA and various Nme2Cas9.

FIG. 17E shows serum TTR protein knockdown following treatment with pgRNA and various Nme2Cas9.

FIG. 18 shows mean percent editing in mouse liver following treatment with pgRNA and various Nme2Cas9.

FIG. 19 shows mean percent editing in mouse liver following treatment with various base editors.

FIG. 20 shows mean percent editing at the HEK3 locus in human hepatoma (Huh7) following treatment with various modified pgRNAs and SpyCas9 mRNA.

### DETAILED DESCRIPTION

Provided herein are guide RNAs (gRNAs) comprising an internal linker for use in gene editing methods. Examples of sequences of engineered and tested gRNAs are shown in Tables 2A-2B.

Certain of the gRNAs provided herein are dual guide RNAs (dgRNAs) comprising an internal linker for use in gene editing methods.

Certain of the gRNAs provided herein are single guide RNAs (sgRNAs) comprising an internal linker for use in gene editing methods.

This disclosure further provides uses of these gRNAs (e.g., sgRNA, dgRNA, or crRNA) to alter the genome of a target nucleic acid in vitro (e.g., cells cultured in vitro for use in ex vivo therapy or other uses of genetically edited cells) or in a cell in a subject such as a human (e.g., for use in in vivo therapy).

sgRNA designations are sometimes provided with one or more leading zeroes immediately following the G. This does not affect the meaning of the designation. Thus, for example, G000282, G0282, G00282, and G282 refer to the same sgRNA. Similarly, crRNA and or trRNA designations are sometimes provided with one or more leading zeroes immediately following the CR or TR, respectively, which does not affect the meaning of the designation. Thus, for example, CR000100, CR00100, CR0100, and CR100 refer to the same crRNA, and TR000200, TR00200, TR0200, and TR200 refer to the same trRNA.

### I. Definitions

The articles “a” and “an” are used herein to refer to one or to more than one (i.e., to at least one) of the grammatical object of the article. By way of example, “an element” means one element or more than one element, e.g., a plurality of elements.

The term “including” is used herein to mean, and is used interchangeably with, the phrase “including but not limited to”.

The term “or” is used herein to mean, and is used interchangeably with, the term “and/or,” unless context clearly indicates otherwise. For example, “sense strand or antisense strand” is understood as “sense strand or antisense strand or sense strand and antisense strand.”

The term “about” is used herein to mean within the typical ranges of tolerances in the art. For example, “about” can be understood as about 2 standard deviations from the mean. In certain embodiments, about means +10%. In certain embodiments, about means +5%, +2%, or +1%. When about is present before a series of numbers or a range, it is understood that “about” can modify each of the numbers in the series or range. At the very least, and not as an attempt to limit the application of the doctrine of equivalents to the scope of the claims, each numerical parameter should at least be construed in light of the number of reported significant digits and by applying ordinary rounding techniques.

The term “at least” prior to a number or series of numbers is understood to include the number adjacent to the term “at least”, and all subsequent numbers or integers that could logically be included, as clear from context. For example, the number of nucleotides in a nucleic acid molecule must be an integer. For example, “at least 17 nucleotides of a 20 nucleotide nucleic acid molecule” means that 17, 18, 19, or 20 nucleotides have the indicated property. When at least is present before a series of numbers or a range, it is understood that “at least” can modify each of the numbers in the series or range.

As used herein, “no more than” or “less than” is understood as the value adjacent to the phrase and logical lower values or integers, as logical from context, to zero. For example, a duplex region of “no more than 2 nucleotide base pairs” has a 2, 1, or 0 nucleotide base pairs. When “no more than” or “less than” is present before a series of numbers or a range, it is understood that each of the numbers in the series or range is modified.

As used herein, ranges include both the upper and lower limit.

As used herein, it is understood that when the maximum amount of a value is represented by 100% (e.g., 100% inhibition) that the value is limited by the method of detection. For example, 100% inhibition is understood as inhibition to a level below the level of detection of the assay.

“Editing efficiency” or “editing percentage” or “percent editing” as used herein is the total number of sequence reads with insertions, deletions, or base changes of nucleotides into the target region of interest over the total number of sequence reads following cleavage or nicking by a Cas RNP.

“Regions” as used herein describes portions of nucleic acids. Regions may also be referred to as “modules” or “domains.” Regions of an sgRNA may perform particular functions, e.g., in directing endonuclease activity of the RNP, for example as described in Briner A E et al., Molecular Cell 56:333-339 (2014), or have predicted structures. Exemplary regions of an sgRNA are described in Table 3.

“Hairpin” or “hairpin structure” as used herein describes a duplex of nucleic acids that is created when a nucleic acid strand folds and forms base pairs with another section of the same strand. A hairpin may form a structure that comprises a loop or a U-shape. In some embodiments, a hairpin may be comprised of an RNA loop. Hairpins can be formed with two complementary sequences in a single nucleic acid molecule bind together, with a folding or wrinkling of the molecule. In some embodiments, hairpins comprise stem or stem loop structures. In some embodiments, a hairpin comprises a loop and a stem. As used herein, when two hairpins are present in a gRNA, a “hairpin region” can refer to hairpin 1 and hairpin 2 and the intervening sequence (e.g., “n”) between hairpin 1 and hairpin 2 of a conserved portion of an sgRNA.

As used herein, “form a duplex portion” is understood as being capable of forming an uninterrupted duplex portion or predicted to form an uninterrupted duplex portion, e.g., by base pairing. A duplex portion may comprise two complementary sequences, e.g., a first hairpin stem region and a second hairpin stem region complementary to the first. As used herein, a duplex portion has a length of at least 2 base pairs. A duplex portion optionally comprises 2-10 base pairs, and the two strands that form the duplex portion may be joined, for example, by a nucleotide loop. Base pairing in a duplex can include Watson-Crick base pairing, optionally in combination with base stacking. As used herein, a duplex portion can include a single nucleotide discontinuity on one strand wherein each contiguous nucleotide on one strand is based paired with a nucleotide on the complementary strand which may have a discontinuity of one non-base paired nucleotide, e.g., as in nucleotide 96 of SEQ ID NO: 500 in hairpin 1, wherein the discontinuity is flanked immediately 5′ and 3′ with Watson-Crick base pairs. This is distinct from non-paired nucleotides 36 and 65 in the repeat-anti-repeat region, and non-paired nucleotides 106-108 and 139 in hairpin 2, which constitute a discontinuity resulting in two duplex portions, as defined herein. RNA structures are well known in the art and tools are available for structural prediction of RNAs (see, e.g., Sato et al., Nature Comm. 12:941 (2021); RNAstructure at ma.urmc.rochester.edu/RNAstructureWeb/Servers/Predict1/Predicti.html and RNAfold WebServer at rna.tbi.univie.ac.at/cgi-bin/RNAWebSuite/RNAfold.cgi). Bridging lengths and structural flexibility required to permit a fold and form a loop to allow nucleobases to come into sufficiently close proximity to base pair are well known in the art.

As used herein, an “RNA-guided DNA binding agent” means a polypeptide or complex of polypeptides having RNA and DNA binding activity, or a DNA-binding subunit of such a complex, wherein the DNA binding activity is sequence-specific and depends on the sequence of the RNA. Exemplary RNA-guided DNA binding agents include Cas cleavases (which have double strand cleaving activity), Cas nickases (which have single strand cleaving activity), and inactivated forms thereof (“dCas DNA binding agents”). “Cas nuclease”, as used herein, encompasses Cas cleavases, Cas nickases, and dCas DNA binding agents. The dCas DNA binding agent may be a dead nuclease comprising non-functional nuclease domains (RuvC or HNH domain). In some embodiments the Cas cleavase or Cas nickase encompasses a dCas DNA binding agent modified to permit DNA cleavage, e.g., via fusion with a FokI domain. In some embodiments, the RNA-guided DNA binding agent has nuclease activity, e.g., cleavase or nickase activity.

“Ribonucleoprotein” (RNP) or “RNP complex” as used herein describes an sgRNA, for example, together with a nuclease, such as a Cas protein. In some embodiments, the RNP comprises Cas9 and gRNA (e.g., sgRNA, dgRNA, or crRNA). In some embodiments, the guide RNA guides the nuclease such as Cas9 to a target sequence, and the guide RNA hybridizes with and the agent binds to the target sequence; in cases where the nuclease or Cas protein is a cleavase or nickase, binding can be followed by cleaving or nicking.

“Stem loop” as used herein describes a secondary structure of nucleotides that form a base-paired “stem” that ends in a loop of unpaired nucleic acids. A stem may be formed when two regions of the same nucleic acid strand are at least partially complementary in sequence when read in opposite directions. “Loop” as used herein describes a region of nucleotides that do not base pair (i.e., are not complementary) that may cap a stem. A “tetraloop” describes a loop of 4 nucleotides. As used herein, the upper stem of an sgRNA may comprise a tetraloop.

“Substituted” or “Substitution” as used herein with respect to a polynucleotide refers to an alteration of a nucleobase, e.g., nucleotide substitution, that changes its preferred base for Watson-Crick pairing. When a certain region of a guide RNA is “unsubstituted” as used herein (e.g., SEQ ID NOs: 200-210 and 500-501 as shown in Table 1A), the sequence of the region can be aligned to that of the corresponding conserved portion of, e.g., a spyCas9 sgRNA (SEQ ID NO: 400) or of any other gRNAs (e.g., part of SEQ ID NO: 200-210 and 500-501) with gaps and matches only (i.e., no mismatches), where bases are considered to match if they have the same preferred standard partner base (A, C, G, or T/U) for Watson-Crick pairing or can form a duplex by base stacking.

As used herein, a “conservative substitution” with respect to a polynucleotide refers to an alteration of a nucleobase means exchanging positions of base paired nucleotides such that base pairings may be maintained. For example, a G-C pair becomes a C-G pair, an A-U pair for a U-A pair, or other natural or modified base pairing.

As used herein, “substituted” and the like, in regard to unpaired nucleotides, e.g., loops of the repeat/anti-repeat, hairpin 1, or hairpin 2 regions, i.e., nucleotides 49-52, 87-90, and 122-125 in SEQ ID NO: 500, respectively, or other unpaired nucleotides, is the replacement of one or more nucleotides, e.g., 1, 2, 3, or 4 nucleotides, of the nucleotide sequence with a different nucleotide that does not interfere with the formation of a structure by the unpaired nucleotides, e.g., a bulge, a loop, to permit formation of the one or more duplex portions, e.g., in the repeat/anti-repeat, hairpin 1, or hairpin 2 regions.

As used herein, “substituted” and the like, in regard to an internal linker, is the replacement of at least 1, preferably at least 2 nucleotides with an internal linker. In certain embodiments, the internal linker has approximately the same predicted bridging length as the number of nucleotides replaced by the linker. In certain embodiments, the internal linker is shorter than the predicted bridging length of the number of nucleotides replaced by the linker. In certain embodiments, the internal linker is longer than the predicted bridging length of the number of nucleotides replaced by the linker. In certain embodiments, the internal linker further substitutes for a portion of the duplex portion of a repeat/anti-repeat portion of a gRNA. In certain embodiments, the internal linker substitutes for a portion of the loop portion of a stem loop in the gRNA. In certain embodiments, the internal linker substitutes for a portion of the duplex portion of a stem loop in the gRNA.

As used herein, an “unlinked portion of a gRNA” with reference to a gRNA comprising an internal linker is a molecule comprising only the nucleotides on one side or the other of the linker and optionally the linker itself or a part thereof. It may also comprise a reactive moiety at the end of the nucleotide sequence, linker or part thereof, or a quenched version of the reactive moiety.

“Guide RNA”, “gRNA”, and “guide” are used herein interchangeably to refer to either a crRNA (also known as CRISPR RNA), or the combination of a crRNA and a trRNA (also known as tracrRNA). The crRNA and trRNA may be associated as a single RNA molecule (single guide RNA, sgRNA) or in two separate RNA molecules (dual guide RNA, dgRNA). “Guide RNA” or “gRNA” refers to each type. The trRNA may be a naturally-occurring sequence, or a trRNA sequence with modifications or variations compared to naturally-occurring sequences. Guide RNAs can include modified RNAs as described herein. Unless otherwise clear from context, a guide RNA as used herein includes at least one internal linker.

“Internal linker” as used herein describes a non-nucleotide segment joining two nucleotides within a guide RNA. If the gRNA contains a spacer region, the internal linker is located outside of the spacer region (e.g., in the scaffold or conserved region of the gRNA). For Type V guides, it is understood that the last hairpin is the only hairpin in the structure, i.e., the repeat-anti-repeat region. As used herein, the linker is a non-nucleotide linker.

As used herein the term “aliphatic” refers to nonaromatic hydrocarbon compounds in which the constituent carbon atoms can be straight-chain, cyclic or branched chain; saturated or unsaturated. In certain embodiments, aliphatic also includes heterocyclic hydrocarbons. Cyclic and heterocyclic hydrocarbons refer to ring structures in which constituent carbon atoms, along with any heteratoms in a heterocyclic group form the ring. The cyclic and heterocyclic hydrocarbons may also contain single, double or triple bonds. C₁₋ₓ aliphatic refers to an aliphatic group having from 1 to x constituent carbon atoms. An aliphatic group may form one or more chemical bonds to other moieties through any of its constituent carbon atoms. Aliphatic groups may be monovalent or divalent as determined by the context in which the term is used.

As used herein the term “alkylene” refers to a saturated bivalent aliphatic chain, which may be straight or branched. Typical alkylene radicals include, but are not limited to: methylene (CH₂) 1,2-ethyl (CH₂CH₂), 1,3-propyl (CH₂CH₂CH₂), 1,4-butyl (CH₂CH₂CH₂CH₂), and the like.

As used herein the term “alkenylene” refers to a bivalent aliphatic chain that is at least partially unsaturated (e.g., containing at least one double bond), which may be straight or branched. Typical alkenylene radicals include, but are not limited to: 1,2-ethylene (CH═CH).

As used herein the term “hydrogen-bond acceptor” refers to a substituent comprising a heteroatom capable of forming a hydrogen bond. H-bond acceptors may be monovalent or divalent as determined by the context in which the term is used. H-bond acceptors include substituents comprising oxygen, sulfur, or phosphorus, or substituents comprising hydroxy, alkoxy, thiol, ether, thioether, carbonyl, amides, carbonates, carbamates, phosphate, phosphorothioate, phosphonate, sulfate, or sulfonate or for example, —O—, —OH, —OR, —ROR, —S—, —SH, —SR, —NH—, —NR—, —C(O)—R, —C(O)—O—, —OC(O)O—, —C(O)—OR, —OC(O)—OR, —C(O)—H, —C(O)—OH, —C(O)—NR—, —OC(O)—NR—, —NC(O)—NR—, —OPO₃, —PO₃, —RPO₃, —P(O)₂O—, —OP(O)₂O—, —OP(R)(O)O—, —OP(O)(S)O—, —S(O)₂—R, —S(O)₂—OR, —RS(O)₂—R, —RS(O)₂—OR, —S(O)₂—, —SO₃.

The “bridging length” of an internal linker as used herein refers to the distance or number of atoms in the shortest chain of atoms on the pathway from the first atom of the linker (bound to a 3′ substituent, such as an oxygen or phosphate, of the preceding nucleotide to the last atom of the linker (bound to a 5′ substituent, such as an oxygen or phosphate) of the following nucleotide) (e.g., from ˜ to #in the structure of Formula (I) described below). Approximate predicted bridging lengths for various linkers are provided in a table below.

In some embodiments, the gRNA (e.g., sgRNA) comprises a “guide region”, which is sometimes referred to as a “spacer” or “spacer region,” for example, in Briner A E et al., Molecular Cell 56:333-339 (2014) for sgRNA (but applicable herein to all guide RNAs). The guide region or spacer region is also sometimes referred to as a “variable region,” “guide domain” or “targeting domain.” In some embodiments, a “guide region” immediately precedes a “conserved portion of an sgRNA” at its 5′ end, and in some embodiments the sgRNA is shortened. An exemplary “conserved portion of an sgRNA” is shown in Tables 3A-B. In some embodiments, a “guide region” comprises a series of nucleotides at the 5′ end of a crRNA

As used herein, “repeat-anti-repeat region” is understood as the portion of the guide corresponding to the duplex or duplexes formed by the crRNA and the trRNA sequences in a guide RNA. In a single guide RNA, the trRNA and crRNA sequences are optionally truncated prior to covalent linkage. The exact position of the truncation can vary. The covalent linkage is routinely a short RNA sequence to allow the formation of a hairpin, typically a stem-loop structure.

A numeric position or range in the guide RNA refers to the position as determined from the 5′ end unless another point of reference is specified; for example, “nucleotide 5” in a guide RNA is the 5^{th} nucleotide from the 5′ end; or “nucleotides 5-8” refers to 4 nucleotides beginning with the 5^{th} nucleotide from the 5′ end and ending with the 8^{th} nucleotide towards the 3′ end.

In some embodiments, a gRNA comprises nucleotides that “match the modification pattern” at corresponding or specified nucleotides of a gRNA described herein. This means that the nucleotides matching the modification pattern have the same modifications (e.g., phosphorothioate, 2′-fluoro, 2′-OMe, etc.) as the nucleotides at the corresponding positions of the gRNA described herein, regardless of whether the nucleobases at those positions match. For example, if in a first gRNA, nucleotides 5 and 6, respectively, have 2′-OMe and phosphorothioate modifications, then this gRNA has the same modification pattern at nucleotides 5 and 6 as a second gRNA that also has 2′-OMe and phosphorothioate modifications at nucleotides 5 and 6, respectively, regardless of whether the nucleobases at positions 5 and 6 are the same or different in the first and second gRNAs. However, a 2′-OMe modification at nucleotide 6 but not nucleotide 7 is not the same modification pattern at nucleotides 6 and 7 as a 2′-OMe modification at nucleotide 7 but not nucleotide 6. Similarly, a modification pattern that matches at least 75% of the modification pattern of a gRNA described herein means that at least 75% of the nucleotides have the same modifications as the corresponding positions of the gRNA described herein. Corresponding positions may be determined by pairwise or structural alignment.

A “conserved region” of a S. pyogenes Cas9 (“spyCas9” (also referred to as “spCas9”)) sgRNA” is shown in Tables 3A-B. The first row shows the numbering of the nucleotides; the second row shows the sequence (e.g., SEQ ID NO: 400); and the third row shows the regions.

As used herein, a “shortened” region in a gRNA is a region in a conserved portion of a gRNA that lacks at least 1 nucleotide compared to the corresponding region in a conserved portion of an unmodified gRNA (see, e.g., FIG. 11 (SEQ ID NO: 400) or Tables 3A-B). Under no circumstances does “shortened” imply any particular limitation on a process or manner of production of the gRNA. In some embodiments, a gRNA comprises a shortened hairpin 1 region, wherein (i) the shortened hairpin 1 region lacks 6-8 nucleotides; and (A) one or more of positions H1-1, H1-2, or H1-3 is deleted or substituted relative to SEQ ID NO: 400 or (B) one or more of positions H1-6 through H1-10 is substituted relative to SEQ ID NO: 400; or (ii) the shortened hairpin 1 region lacks 9-10 nucleotides including H1-1 or H1-12; or (iii) the shortened hairpin 1 region lacks 5-10 nucleotides and one or more of positions N18, H1-12, or N is substituted relative to SEQ ID NO: 400 (see Table 3A). In some embodiments, a non-spyCas9 gRNA comprises a shortened hairpin 1 region that lacks 6-8 nucleotides and in which one or more positions corresponding to H1-1, H1-2, or H1-3 in SEQ ID NO: 400 as determined, for example, by pairwise or structural alignment, is deleted or substituted, one or more of positions corresponding to H1-6 through H1-10 in SEQ ID NO: 400 as determined, for example, by pairwise or structural alignment, is substituted. In some embodiments, a non-spyCas9 gRNA comprises a shortened hairpin 1 region that lacks 9-10 nucleotides including nucleotides corresponding to H1-1 or H1-12 in SEQ ID NO: 400 as determined, for example, by pairwise or structural alignment. In some embodiments, a non-spyCas9 gRNA comprises a shortened hairpin 1 region that lacks 5-10 nucleotides and one or more positions corresponding to N18, H1-12, or N in SEQ ID NO: 400 as determined, for example, by pairwise or structural alignment, is substituted. In some embodiments, a gRNA comprises a shortened upper stem region, wherein the shortened upper stem region lacks 1-6 nucleotides.

As used herein, a “YA site” refers to a 5′-pyrimidine-adenine-3′ dinucleotide. For clarification, a “YA site” in an original sequence that is altered by modifying a base is still considered a (modified) YA site in the resulting sequence, regardless of the absence of a literal YA dinucleotide. A “conserved region YA site” is present in the conserved region of an sgRNA. A “guide region YA site” is present in the guide region of an sgRNA. An unmodified YA site in an sgRNA may be susceptible to cleavage by RNase-A like endonucleases, e.g., RNase A. In certain embodiments, a YA site is modified to reduce susceptibility to RNAse A by a 2′ sugar modification, e.g., 2′OMe, 2′F, or backbone modification, e.g., phosphorothioate linkage. In certain embodiments, a YA site is modified by modifying the base so a YA sequence is no longer present.

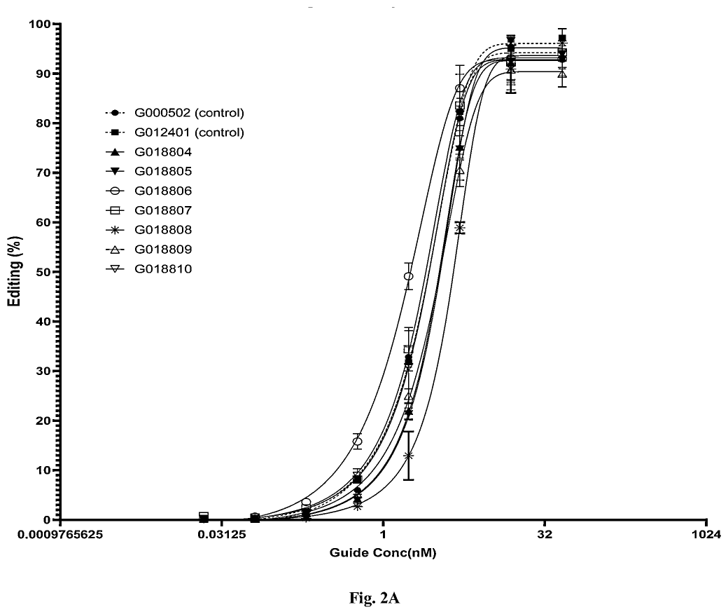

As discussed herein, positions of nucleotides corresponding to those described with respect to spyCas9 gRNA can be identified in another gRNA with sequence or structural similarity by pairwise or structural alignment. Structural alignment is useful where molecules share similar structures despite considerable sequence variation. For example, spyCas9 and Staphylococcus aureus Cas9 (“SauCas9”) have divergent sequences, but significant structural alignment. See, e.g., FIG. 2(F) from Nishimasu et al., Cell 162(5): 1113-1126 (2015). Structural alignment can be used to identify nucleotides in a SauCas9 or other sgRNA that correspond to particular positions, such as positions H1-1, H1-2, or H1-3, positions H1-6 through H1-10, position H1-12, or positions N18 or N of the conserved portion of a spyCas9 sgRNA (e.g., SEQ ID NO: 400) (see Table 3A).

Structural alignment involves identifying corresponding residues across two (or more) sequences by (i) modeling the structure of a first sequence using the known structure of the second sequence or (ii) comparing the structures of the first and second sequences where both are known, and identifying the residue in the first sequence most similarly positioned to a residue of interest in the second sequence. Corresponding residues are identified in some algorithms based on distance minimization given position (e.g., nucleobase position 1 or the 1′ carbon of the pentose ring for polynucleotides, or alpha carbons for polypeptides) in the overlaid structures (e.g., what set of paired positions provides a minimized root-mean-square deviation for the alignment). When identifying positions in a non-spyCas9 gRNA corresponding to positions described with respect to spyCas9 gRNA, spyCas9 gRNA can be the “second” sequence. Where a non-spyCas9 gRNA of interest does not have an available known structure, but is more closely related to another non-spyCas9 gRNA that does have a known structure, it may be most effective to model the non-spyCas9 gRNA of interest using the known structure of the closely related non-spyCas9 gRNA, and then compare that model to the spyCas9 gRNA structure to identify the desired corresponding residue in the non-spyCas9 gRNA of interest. There is an extensive literature on structural modeling and alignment for proteins; representative disclosures include U.S. Pat. Nos. 6,859,736; 8,738,343; and those cited in Aslam et al., Electronic Journal of Biotechnology 20 (2016) 9-13. For discussion of modeling a structure based on a known related structure or structures, see, e.g., Bordoli et al., Nature Protocols 4 (2009) 1-13, and references cited therein. See also FIG. 2(F) from Nishimasu et al., Cell 162(5): 1113-1126 (2015) for alignment of nucleic acid. Further, extensive structural studies have been performed on Cas nucleases complexes with their guide RNAs, see, e.g., Jiang et al., Science. 2015 Jun. 26; 348(6242):1477-81; Anders et al., Nature. 2014 Sep. 25; 513(7519):569-73; Zhu et al., Nat Struct Mol Biol. 2019 August; 26(8):679-685; Nishimasu et al., Cell. 2014 Feb. 27; 156(5):935-49; Nishimasu et al., Cell. 2015 Aug. 27; 162(5):1113-26; Hirano et al., Nat Commun. 2019 Apr. 29; 10(1):1968; Fuchsbauer et al., Mol Cell. 2019 Dec. 19; 76(6):922-937; Zhang et al., Nat Catal 3, 813-823 (2020); Yamada et al., Mol Cell. 2017 Mar. 16; 65(6):1109-1121; Hirano et al., Cell. 2016 Feb. 25; 164(5):950-61; Gao et al., Cell Res. 2016 August; 26(8):901-13; and Stella et al., Nature. 2017 Jun. 22; 546(7659):559-563. Erratum in: Nature. 2017 Jul. 27; 547(7664):476; Qiao et al., Biotechnol Bioeng. 2021 May 8 (doi: 10.1002/bit.27813 Epub ahead of print). Provided with these co-structures, the location of duplex regions, hairpins, and contacts between the nucleases and their guides can be readily determined.

A “target sequence” as used herein refers to a sequence of nucleic acid to which the guide region directs a nuclease for cleavage. In some embodiments, a spyCas9 protein may be directed by a guide region to a target sequence by the nucleotides present in the guide region.

As used herein, the “5′ end” refers to the first nucleotide of the gRNA (including a dgRNA (typically the 5′ end of the crRNA of the dgRNA), sgRNA), in which the 5′ position is not linked to another nucleotide.

As used herein, a “5′ end modification” refers to a gRNA comprising a guide region having modifications in one or more of the one (1) to about seven (7) nucleotides at its 5′ end, optionally wherein the first nucleotide (from the 5′ end) of the gRNA is modified.

As used herein, the “3′ end” refers to the end or terminal nucleotide of a gRNA, in which the 3′ position is not linked to another nucleotide. In some embodiment, the 3′ end is in the 3′ tail. In some embodiments, the 3′ end is in the conserved portion of an gRNA.

As used herein, a “3′ end modification” refers to a gRNA having modifications in one or more of the one (1) to about seven (7) nucleotides at its 3′ end, optionally wherein the last nucleotide (i.e., the 3′ most nucleotide) of the gRNA is modified. If a 3′ tail is present, the 1 to about 7 nucleotides may be within the 3′ tail. If a 3′ tail is not present, the 1 to about 7 nucleotides may be within the conserved portion of a sgRNA.

The “last,” “second to last,” “third to last,” etc., nucleotide refers to the 3′ most, second 3′ most, third 3′ most, etc., nucleotide, respectively in a given sequence. For example, in the sequence 5′-AAACTG-3′, the last, second to last, and third to last nucleotides are G, T, and C, respectively. The phrase “last 3 nucleotides” refers to the last, second to last, and third to last nucleotides; more generally, “last N nucleotides” refers to the last to the Nth to last nucleotides, inclusive. “Third nucleotide from the 3′ end of the 3′ terminus” is equivalent to “third to last nucleotide.” Similarly, “third nucleotide from the 5′ end of the 5′ terminus” is equivalent to “third nucleotide at the 5′ terminus.”

As used herein, a “protective end modification” (such as a protective 5′ end modification or protective 3′ end modification) refers to a modification of one or more nucleotides within seven nucleotides of the end of an sgRNA that reduces degradation of the sgRNA, such as exonucleolytic degradation. In some embodiments, a protective end modification comprises modifications of at least two or at least three nucleotides within seven nucleotides of the end of the sgRNA. In some embodiments, the modifications comprise phosphorothioate linkages, 2′ modifications such as 2′-OMe or 2′-fluoro, 2′-H (DNA), ENA, UNA, or a combination thereof. In some embodiments, the modifications comprise phosphorothioate linkages and 2′-OMe modifications. In some embodiments, at least three terminal nucleotides are modified, e.g., with phosphorothioate linkages or with a combination of phosphorothioate linkages and 2′-OMe modifications. Modifications known to those of skill in the art to reduce exonucleolytic degradation are encompassed.

In some embodiments, a “3′ tail” comprising 1-20 nucleotides, optionaly 1-7 nucleotides, or 1 nucleotide, and follows the conserved portion of a sgRNA at its 3′ end. In certain embodiments, the terminal base is uracil. In certain embodiments, the tail is a one nucleotide and the terminal base is uracil.

“Cas nuclease”, also called “Cas protein”, as used herein, encompasses Cas cleavases, Cas nickases, and dCas DNA binding agents. Cas cleavases/nickases and dCas DNA binding agents include a Csm or Cmr complex of a type III CRISPR system, the Cas10, Csm1, or Cmr2 subunit thereof, a Cascade complex of a type I CRISPR system, the Cas3 subunit thereof, and Class 2 Cas nucleases; a type V CRISPR system including the Cas12, or a subunit thereof, such as a Cas12a (Cpf1) or a Cas12e (CasX); and a type VI CRISPR system, including Cas13d. As used herein, a “Class 2 Cas nuclease” is a single-chain polypeptide with RNA-guided DNA binding activity, such as a Cas9 nuclease or a Cpf1 nuclease. Class 2 Cas nucleases include Class 2 Cas cleavases and Class 2 Cas nickases (e.g., H840A, D10A, or N863A variants), which further have RNA-guided DNA cleavases or nickase activity, and Class 2 dCas DNA binding agents, in which cleavase/nickase activity is inactivated. Class 2 Cas nucleases include, for example, Cas9, Cpf1, C2cl, C2c2, C2c3, HF Cas9 (e.g., N497A, R661A, Q695A, Q926A variants), HypaCas9 (e.g., N692A, M694A, Q695A, H698A variants), eSPCas9(1.0) (e.g, K810A, K1003A, R1060A variants), and eSPCas9(1.1) (e.g., K848A, K1003A, R1060A variants) proteins and modifications thereof. Cpf1 protein, Zetsche et al., Cell, 163: 1-13 (2015), is homologous to Cas9, and contains a RuvC-like nuclease domain. Cpf1 sequences of Zetsche are incorporated by reference in their entirety. See, e.g., Zetsche, Tables Si and S3. “Cas9” encompasses Spy Cas9, the variants of Cas9 listed herein, and equivalents thereof. See, e.g., Makarova et al., Nat Rev Microbiol, 13(11): 722-36 (2015); Shmakov et al., Molecular Cell, 60:385-397 (2015).

Class 2 CRISPR systems are characterized by having a monomeric endonuclease, rather than a multimeric nuclease. Class 2 CRISPR systems include Type II and Type V systems.

Type II systems include a relatively large Cas9 endonuclease having an RNA recognition domain, two nuclease domains, an HNH domain connected to a RuvC domain by an arginine-rich helix bridge, and a protospacer adjacent motif (PAM) interacting domain. The guide RNAs tend to be relatively long, i.e., single guide RNAs are typically about 100 nucleotides in length, or longer, and have been demonstrated by a number of functional studies to include multiple duplex regions and hairpins 3′ to the spacer (targeting domain region) including the repeat-anti-repeat region and a second hairpin region, typically containing one or two predicted hairpin structures.

Type II Cas9 endonucleases include Type II-A Cas9 endonucleases, e.g., S. pyogenes (Spy Cas9), and Type II-C Cas9 endonucleases, e.g., C. jejuni (Cje), R. palustris (Rpa), R. rubrum (Rru), A. naeslundii (Ana), and C. diphtheriae (Cdi).

Type V systems are characterized by relatively smaller nucleases and guides. The nucleases have a single DNA recognition lobe (REC) and a single nuclease (NUC) lobe. The guides occur naturally as a single RNA of about 40-45 nucleotides in length and include a single hairpin repeat-anti-repeat region about 20 nucleotides in length followed by a 23-25 nucleotide spacer region. Type V systems include Francisella novicida Cpf1 (FnCpf1), Lachnospiraceae bacterium Cpf1 (LbCpf1), and Acidaminococcus sp. Cpf1 (AsCpf1/Cas 12a).

As used herein, a first sequence is considered to “comprise a sequence with at least X % identity to” a second sequence if an alignment of the first sequence to the second sequence shows that X % or more of the positions of the second sequence in its entirety are matched by the first sequence. For example, the sequence AAGA comprises a sequence with 100% identity to the sequence AAG because an alignment would give 100% identity in that there are matches to all three positions of the second sequence. The differences between RNA and DNA (generally the exchange of uridine for thymidine or vice versa) and the presence of nucleoside analogs such as modified uridines do not contribute to differences in identity or complementarity among polynucleotides as long as the relevant nucleotides (such as thymidine, uridine, or modified uridine) have the same complement (e.g., adenosine for all of thymidine, uridine, or modified uridine; another example is cytosine and 5-methylcytosine, both of which have guanosine or modified guanosine as a complement). Thus, for example, the sequence 5′-AXG where X is any modified uridine, such as pseudouridine, N1-methyl pseudouridine, or 5-methoxyuridine, is considered 100% identical to AUG in that both are perfectly complementary to the same sequence (5′-CAU). Exemplary alignment algorithms are the Smith-Waterman and Needleman-Wunsch algorithms, which are well-known in the art. One skilled in the art will understand what choice of algorithm and parameter settings are appropriate for a given pair of sequences to be aligned; for sequences of generally similar length and expected identity >50% for amino acids or >75% for nucleotides, the Needleman-Wunsch algorithm with default settings of the Needleman-Wunsch algorithm interface provided by the EBI at the www.ebi.ac.uk web server is generally appropriate.

“mRNA” is used herein to refer to a polynucleotide that is RNA or modified RNA and comprises an open reading frame that can be translated into a polypeptide (i.e., can serve as a substrate for translation by a ribosome and amino-acylated tRNAs). mRNA can comprise a phosphate-sugar backbone including ribose residues or analogs thereof, e.g., 2′-methoxy ribose residues. In some embodiments, the sugars of a nucleic acid phosphate-sugar backbone consist essentially of ribose residues, 2′-methoxy ribose residues, or a combination thereof. In general, mRNAs do not contain a substantial quantity of thymidine residues (e.g., 0 residues or fewer than 30, 20, 10, 5, 4, 3, or 2 thymidine residues; or less than 10%, 9%, 8%, 7%, 6%, 5%, 4%, 4%, 3%, 2%, 1%, 0.5%, 0.2%, or 0.1% thymidine content). An mRNA can contain modified uridines at some or all of its uridine positions. In some embodiments, a modified mRNAs comprises at least one nucleotide in which one or more of the phosphate, sugar, or nucleobase differ from that of a standard adenosine, cytidine, guanidine, or uridine nucleotide.

As used herein, a “subject” refers to any member of the animal kingdom. In some embodiments, “subject” refers to humans. In some embodiments, “subject” refers to non-human animals. In some embodiments, “subject” refers to primates. In some embodiment, “subject” refers to non-huamn primates. In some embodiments, subjects include, but are not limited to, mammals, birds, reptiles, amphibians, fish, insects, or worms. In certain embodiments, the non-human subject is a mammal (e.g., a rodent, a mouse, a rat, a rabbit, a monkey, a dog, a cat, a sheep, cattle, a primate, or a pig). In some embodiments, a subject may be a transgenic animal, genetically-engineered animal, or a clone. In certain embodiments of the present invention the subject is an adult, an adolescent or an infant. In some embodiments, terms “individual” or “patient” are used and are intended to be interchangeable with “subject” wherein the subject is a human subject.

As used herein, “delivering” and “administering” are used interchangeably, and include ex vivo and in vivo applications.

Co-administration, as used herein, means that a plurality of substances are administered sufficiently close together in time so that the agents act together. Co-administration encompasses administering substances together in a single formulation and administering substances in separate formulations close enough in time so that the agents act together.

As used herein, the phrase “pharmaceutically acceptable” means that which is useful in preparing a pharmaceutical composition that is generally non-toxic and is not biologically undesirable and that are not otherwise unacceptable for pharmaceutical use. Pharmaceutically acceptable generally refers to substances that are non-pyrogenic. Pharmaceutically acceptable can refer to substances that are sterile, especially for pharmaceutical substances that are for injection or infusion.

### II. GUIDE RNAS COMPRISING INTERNAL LINKERS

Provided herein are guide RNAs (gRNAs) comprising an internal linker for use in gene editing methods.

A. Locations/Numbers of Internal Linkers

In some embodiments, the internal linker substitutes for at least 1 nucleotide. In some embodiments, the internal linker substitutes for at least 2 nucleotides. In some embodiments, the internal linker substitutes for at least 3 nucleotides. In some embodiments, the internal linker substitutes for at least 4 nucleotides. In some embodiments, the internal linker substitutes for at least 5 nucleotides. In some embodiments, the internal linker substitutes for at least 6 nucleotides. In some embodiments, the internal linker substitutes for at least 7 nucleotides. In some embodiments, the internal linker substitutes for at least 8 nucleotides. In some embodiments, the internal linker substitutes for at least 9 nucleotides. In some embodiments, the internal linker substitutes for at least 10 nucleotides. In some embodiments, the internal linker substitutes for at least 11 nucleotides. In some embodiments, the internal linker substitutes for at least 12 nucleotides. In some embodiments, the internal linker substitutes for at least 2, 3, 4, 5, 6, 7, 8, 9, 10, 11, 12, 13, 14, 15, 16, 17, 18, 19, 20, 21, 22, 23, 24, 25, 26, 27, or 28 nucleotides of the gRNA. In some embodiments, an internal linker substitutes for at least 28 nucleotides of the gRNA. In some embodiments, an internal linker substitutes for at least 22 nucleotides of the gRNA. In some embodiments, the linker substitutes for at least 2-6 nucleotides. In some embodiments, the linker substitutes for at least 2-4 nucleotides.

In some embodiments, an internal linker substitutes for up to 28 nucleotides of the gRNA. In some embodiments, an internal linker substitutes for up to 22 nucleotides of the gRNA. In some embodiments, an internal linker substitutes for up to 12 nucleotides of the gRNA.

In some embodiments, the internal linker substitutes for 2 nucleotides. In some embodiments, the internal linker substitutes for 3 nucleotides. In some embodiments, the internal linker substitutes for 4 nucleotides. In some embodiments, the internal linker substitutes for 5 nucleotides. In some embodiments, the internal linker substitutes for 6 nucleotides. In some embodiments, the internal linker substitutes for 7 nucleotides. In some embodiments, the internal linker substitutes for 8 nucleotides. In some embodiments, the internal linker substitutes for 9 nucleotides. In some embodiments, the internal linker substitutes for 10 nucleotides. In some embodiments, the internal linker substitutes for 11 nucleotides. In some embodiments, the internal linker substitutes for 12 nucleotides. In some embodiments, the linker substitutes for 2-28 nucleotides. In some embodiments, the linker substitutes for 2-22 nucleotides. In some embodiments, the linker substitutes for 2-12 nucleotides. In some embodiments, the linker substitutes for 2-6 nucleotides. In some embodiments, the linker substitutes for 2-4 nucleotides.

In some embodiments, the internal linker has a bridging length of about 3-30 atoms. In some embodiments, the internal linker has a bridging length of about 6-30 atoms. In some embodiments, the internal linker has a bridging length of about 9-30 atoms. In some embodiments, the internal linker has a bridging length of about 12-30 atoms. In some embodiments, the internal linker has a bridging length of about 15-30 atoms. In some embodiments, the internal linker has a bridging length of about 18-30 atoms. In some embodiments, the internal linker has a bridging length of about 21-30 atoms. In some embodiments, the internal linker has a bridging length of about 12-21 atoms. In some embodiments, the internal linker has a bridging length of about 9-21 atoms. In some embodiments, the internal linker has a bridging length of about 6-12 atoms.

In some embodiments, the internal linker has a bridging length of about 3-30 atoms, and the linker substitutes for at least 4 nucleotides of the gRNA. In some embodiments, the internal linker has a bridging length of about 12-30 atoms, and the linker substitutes for at least 4 nucleotides of the gRNA. In some embodiments, the internal linker has a bridging length of about 12-24 atoms, and the linker substitutes for at least 4 nucleotides of the gRNA. In some embodiments, the internal linker has a bridging length of about 12-21 atoms, and the linker substitutes for at least 4 nucleotides of the gRNA. In some embodiments, the internal linker has a bridging length of about 16-20 atoms, and the linker substitutes for at least 4 nucleotides of the gRNA. In some embodiments, the internal linker has a bridging length of about 15-18 atoms, and the linker substitutes for at least 4 nucleotides of the gRNA.

In some embodiments, the internal linker has a bridging length of about 15 atoms, and the linker substitutes for at least 4 nucleotides of the gRNA. In some embodiments, the internal linker has a bridging length of about 16 atoms, and the linker substitutes for at least 4 nucleotides of the gRNA. In some embodiments, the internal linker has a bridging length of about 17 atoms, and the linker substitutes for at least 4 nucleotides of the gRNA. In some embodiments, the internal linker has a bridging length of about 18 atoms, and the linker substitutes for at least 4 nucleotides of the gRNA. In some embodiments, the internal linker has a bridging length of about 19 atoms, and the linker substitutes for at least 4 nucleotides of the gRNA. In some embodiments, the internal linker has a bridging length of about 20 atoms, and the linker substitutes for at least 4 nucleotides of the gRNA. In some embodiments, the internal linker has a bridging length of about 21 atoms, and the linker substitutes for at least 4 nucleotides of the gRNA. In some embodiments, the internal linker has a bridging length of about 22 atoms, and the linker substitutes for at least 4 nucleotides of the gRNA. In some embodiments, the internal linker has a bridging length of about 23 atoms, and the linker substitutes for at least 4 nucleotides of the gRNA. In some embodiments, the internal linker has a bridging length of about 24 atoms, and the linker substitutes for at least 4 nucleotides of the gRNA. In some embodiments, the internal linker has a bridging length of about 25 atoms, and the linker substitutes for at least 4 nucleotides of the gRNA. In some embodiments, the internal linker has a bridging length of about 26 atoms, and the linker substitutes for at least 4 nucleotides of the gRNA. In some embodiments, the internal linker has a bridging length of about 27 atoms, and the linker substitutes for at least 4 nucleotides of the gRNA. In some embodiments, the internal linker has a bridging length of about 28 atoms, and the linker substitutes for at least 4 nucleotides of the gRNA. In some embodiments, the internal linker has a bridging length of about 29 atoms, and the linker substitutes for at least 4 nucleotides of the gRNA. In some embodiments, the internal linker has a bridging length of about 30 atoms, and the linker substitutes for at least 4 nucleotides of the gRNA.

In some embodiments, the internal linker has a bridging length of about 21 atoms, and the linker substitutes for 4 nucleotides of the gRNA. In some embodiments, the internal linker has a bridging length of about 21 atoms, and the linker substitutes for 6 nucleotides of the gRNA. In some embodiments, the internal linker has a bridging length of about 21 atoms, and the linker substitutes for 8 nucleotides of the gRNA. In some embodiments, the internal linker has a bridging length of about 21 atoms, and the linker substitutes for 4 nucleotides of the gRNA. In some embodiments, the internal linker has a bridging length of about 21 atoms, and the linker substitutes for 10 nucleotides of the gRNA. In some embodiments, the internal linker has a bridging length of about 21 atoms, and the linker substitutes for 12 nucleotides of the gRNA.

In some embodiments, the internal linker has a bridging length of about 18 atoms, and the linker substitutes for 4 nucleotides of the gRNA. In some embodiments, the internal linker has a bridging length of about 18 atoms, and the linker substitutes for 6 nucleotides of the gRNA. In some embodiments, the internal linker has a bridging length of about 18 atoms, and the linker substitutes for 8 nucleotides of the gRNA. In some embodiments, the internal linker has a bridging length of about 18 atoms, and the linker substitutes for 4 nucleotides of the gRNA. In some embodiments, the internal linker has a bridging length of about 18 atoms, and the linker substitutes for 10 nucleotides of the gRNA. In some embodiments, the internal linker has a bridging length of about 18 atoms, and the linker substitutes for 12 nucleotides of the gRNA.

In some embodiments, the internal linker has a bridging length of about 6-18 atoms, optionally about 6-12 atoms, and the linker substitutes for at least 2 nucleotides of the gRNA. In some embodiments, the internal linker has a bridging length of about 9-12 atoms, and the linker substitutes for at least 2 nucleotides of the gRNA. In some embodiments, the internal linker has a bridging length of about 8-10 atoms, and the linker substitutes for at least 2 nucleotides of the gRNA.

In some embodiments, the internal linker has a bridging length of about 6 atoms, and the linker substitutes for at least 2 nucleotides of the gRNA. In some embodiments, the internal linker has a bridging length of about 7 atoms, and the linker substitutes for at least 2 nucleotides of the gRNA. In some embodiments, the internal linker has a bridging length of about 8 atoms, and the linker substitutes for at least 2 nucleotides of the gRNA. In some embodiments, the internal linker has a bridging length of about 9 atoms, and the linker substitutes for at least 2 nucleotides of the gRNA. In some embodiments, the internal linker has a bridging length of about 10 atoms, and the linker substitutes for at least 2 nucleotides of the gRNA. In some embodiments, the internal linker has a bridging length of about 11 atoms, and the linker substitutes for at least 2 nucleotides of the gRNA. In some embodiments, the internal linker has a bridging length of about 12 atoms, and the linker substitutes for at least 2 nucleotides of the gRNA.

In some embodiments, the internal linker has a bridging length of about 9 atoms, and the linker substitutes for 2 nucleotides of the gRNA.

In some embodiments, the internal linker is in a repeat-anti-repeat region of the gRNA. In some embodiments, the internal linker substitutes for at least 3 nucleotides of the repeat-anti-repeat region of the gRNA. In some embodiments, the internal linker substitutes for at least 3, 4, 5, 6, 7, 8, 9, 10, 11, or 12 nucleotides of the repeat-anti-repeat region of the gRNA. In some embodiments, the internal linker substitutes for 3 nucleotides of the repeat-anti-repeat region of the gRNA. In some embodiments, the internal linker substitutes for 4 nucleotides of the repeat-anti-repeat region of the gRNA. In some embodiments, the internal linker substitutes for 5 nucleotides of the repeat-anti-repeat region of the gRNA. In some embodiments, the internal linker substitutes for 6 nucleotides of the repeat-anti-repeat region of the gRNA. In some embodiments, the internal linker substitutes for 7 nucleotides of the repeat-anti-repeat region of the gRNA. In some embodiments, the internal linker substitutes for 8 nucleotides of the repeat-anti-repeat region of the gRNA. In some embodiments, the internal linker substitutes for 9 nucleotides of the repeat-anti-repeat region of the gRNA. In some embodiments, the internal linker substitutes for 10 nucleotides of the repeat-anti-repeat region of the gRNA. In some embodiments, the internal linker substitutes for 11 nucleotides of the repeat-anti-repeat region of the gRNA. In some embodiments, the internal linker substitutes for 12 nucleotides of the repeat-anti-repeat region of the gRNA. In some embodiments, the internal linker substitutes for up to 28 nucleotides in the repeat-anti-repeat region. In some embodiments, the internal linker substitutes for up to 20 nucleotides in the repeat-anti-repeat region. In some embodiments, the internal linker is flanked by nucleotides forming a duplex region of at least 2 base pairs in length. In certain embodiments, the internal linker is not present in a bulge in a repeat-anti-repeat region.

In some embodiments, the internal linker is in a hairpin region of the gRNA. In some embodiments, the internal linker substitutes for at least 2 nucleotides of the hairpin region of the gRNA. In some embodiments, the internal linker substitutes for at least 2, 3, 4, 5, 6, 7, 8, 9, 10, 11, or 12 nucleotides of the hairpin region of the gRNA. In some embodiments, the internal linker substitutes for up to 22 nucleotides of the hairpin region of the gRNA. In some embodiments, the internal linker substitutes for up to 12 nucleotides of the hairpin region of the gRNA.

In some embodiments, the internal linker substitutes for at least 2 nucleotides of the hairpin region of the gRNA. In some embodiments, the internal linker substitutes for at least 4 nucleotides of the hairpin region of the gRNA. In some embodiments, the internal linker substitutes for 6 nucleotides of the hairpin region of the gRNA. In some embodiments, the internal linker substitutes for 8 nucleotides of the hairpin region of the gRNA. In some embodiments, the internal linker substitutes for 10 nucleotides of the hairpin of the gRNA. In some embodiments, the internal linker substitutes for 12 nucleotides of the hairpin region of the gRNA. In some embodiments, the internal linker substitutes for 14 nucleotides of the hairpin region of the gRNA. In some embodiments, the internal linker substitutes for 16 nucleotides of the hairpin region of the gRNA. In some embodiments, the internal linker substitutes for 18 nucleotides of the hairpin region of the gRNA. In some embodiments, the internal linker substitutes for 20 nucleotides of the hairpin region of the gRNA. In some embodiments, the internal linker substitutes for 22 nucleotides of the hairpin of the gRNA. In some embodiments, the internal linker substitutes for up to 22 nucleotides of the hairpin region of the gRNA. In some embodiments, the internal linker substitutes for 2-6 nucleotides of the hairpin region of the gRNA. In some embodiments, the internal linker substitutes for 2-4 nucleotides of the hairpin region of the gRNA. In some embodiments, the internal linker is flanked by nucleotides forming a duplex region of at least 2 base pairs in length. In some embodiments, the internal linker substitutes for all of a hairpin structure in a hairpin region, i.e., a duplex is not formed by the nucleotides flanking the internal linker.

In some embodiments, the internal linker substitutes for 1, 2, 3, 4, 5, or 6 base pairs of the hairpin region of the gRNA. In some embodiments, the internal linker substitutes for 1 base pair of the hairpin region of the gRNA, i.e., for nucleotides predicted to form a base pair in a hairpin structure such that a 1 base pair deletion results in the deletion of two nucleotides and a reduced number of base pairs in the hairpin structure by one. In some embodiments, the internal linker substitutes for 2 base pairs of the hairpin region of the gRNA. In some embodiments, the internal linker substitutes for 3 base pairs of the hairpin region of the gRNA. In some embodiments, the internal linker substitutes for 4 base pairs of the hairpin region of the gRNA. In some embodiments, the internal linker substitutes for 5 base pairs of the hairpin of the gRNA. In some embodiments, the internal linker substitutes for 6 base pairs of the hairpin region of the gRNA. In some embodiments, the internal linker substitutes for 1-12 base pairs of the hairpin region of the gRNA. In some embodiments, the internal linker substitutes for 1-6 base pairs of the hairpin region of the gRNA. In some embodiments, the internal linker substitutes for 1-4 base pairs of the hairpin region of the gRNA. In some embodiments, the internal linker substitutes for up to 12 base pairs of the hairpin region of the gRNA.

In some embodiments, the internal linker is in a nexus region of the gRNA. In some embodiments, the internal linker substitutes for at least 2 nucleotides of the nexus region of the gRNA. In some embodiments, the internal linker substitutes for 1 or 2 nucleotides of the nexus region of the gRNA.

In some embodiments, the internal linker is in a hairpin structure between a first portion of the gRNA and a second portion of the gRNA, wherein the first portion and the second portion together form a duplex portion.

In some embodiments, the gRNA comprises three internal linkers. In some embodiments, the gRNA comprises two internal linkers. In some embodiments, the gRNA comprises one internal linker.

Upper Stem of Repeat-Anti-Repeat Region

In some embodiments, the internal linker in the repeat-anti-repeat region is in a hairpin structure between a first portion and a second portion of the repeat-anti-repeat region, wherein the first portion and the second portion together form a duplex portion.

In some embodiments, the internal linker in the repeat-anti-repeat region substitutes for 2, 3, 4, 5, 6, 7, 8, 9, 10, 11, 12, 13, 14, 15, 16, 17, 18, 19, 20, 21, 22, 23, 24, 25, 26, 27, or 28 nucleotides of the hairpin structure. In some embodiments, the internal linker substitutes for up to 28 nucleotides in the repeat-anti-repeat region. In some embodiments, the internal linker substitutes for up to 20 nucleotides in the repeat-anti-repeat region. In some embodiments, the internal linker substitutes for up to 12 nucleotides in the repeat-anti-repeat region. In some embodiments, the internal linker substitutes for at lesat 4 nucleotides in the repeat-anti-repeat region. In some embodiments, the internal linker substitutes for 4-20 nucleotides in the repeat-anti-repeat region. In some embodiments, the internal linker substitutes for 4-14 nucleotides in the repeat-anti-repeat region. In some embodiments, the internal linker substitutes for 4-6 nucleotides in the repeat-anti-repeat region.

In some embodiments, the internal linker in the repeat-anti-repeat region substitutes for a loop, or part thereof, of the hairpin structure. In some embodiments, the internal linker in the repeat-anti-repeat region substitutes for the loop and the stem, or part thereof, of the hairpin structure. In some embodiments, the internal linker does not substitute for a bulge portion of a repeat-anti-repeat region.

In some embodiments, the internal linker in the repeat-anti-repeat region substitutes for 2, 3, or 4 nucleotides of the loop of the hairpin structure. In some embodiments, the internal linker in the repeat-anti-repeat region substitutes for 2 nucleotides of the loop of the hairpin structure. In some embodiments, the internal linker in the repeat-anti-repeat region substitutes for 3 nucleotides of the loop of the hairpin structure. In some embodiments, the internal linker in the repeat-anti-repeat region substitutes for 4 nucleotides of the loop of the hairpin structure.

In some embodiments, the internal linker in the repeat-anti-repeat region substitutes for the loop of the hairpin structure and at least 1 nucleotide of the stem of the hairpin. In some embodiments, wherein the internal linker in the repeat-anti-repeat region substitutes for the loop of the hairpin and 1, 2, 3, 4, 5, 6, 7, 8, 9, 10, 11, 12, 13, 14, 15, 16, 17, 18, 19, 20, 21, 22, 23, or 24 nucleotides of the stem of the hairpin. In some embodiments, the internal linker in the repeat-anti-repeat region substitutes for the loop of the hairpin and at least 2 nucleotides of the stem of the hairpin. In some embodiments, the internal linker in the repeat-anti-repeat region substitutes for the loop of the hairpin and 2-24 nucleotides of the stem of the hairpin. In some embodiments, the internal linker in the repeat-anti-repeat region substitutes for the loop of the hairpin and 2-18 nucleotides of the stem of the hairpin. In some embodiments, the internal linker in the repeat-anti-repeat region substitutes for the loop of the hairpin and 2-8 nucleotides of the stem of the hairpin.

In some embodiments, the internal linker in the repeat-anti-repeat region substitutes for the loop of the hairpin structure and 2, 4, 6, 8, 10, 12, 14, 16, 18, 20, 22, or 24 nucleotides of the stem of the hairpin structure. In some embodiments, the internal linker in the repeat-anti-repeat region substitutes for the loop of the hairpin structure and 2, 4, 6, 8, 10, 12, or 14 nucleotides of the stem of the hairpin structure. In some embodiments, the internal linker in the repeat-anti-repeat region substitutes for the loop of the hairpin structure and 2, 4, 6, or 8 nucleotides of the stem of the hairpin structure. In some embodiments, the internal linker in the repeat-anti-repeat region substitutes for the loop of the hairpin structure and 2 nucleotides of the stem of the hairpin structure. In some embodiments, the internal linker in the repeat-anti-repeat region substitutes for the loop of the hairpin structure and 4 nucleotides of the stem of the hairpin structure. In some embodiments, the internal linker in the repeat-anti-repeat region substitutes for the loop of the hairpin structure and 6 nucleotides of the stem of the hairpin structure. In some embodiments, the internal linker in the repeat-anti-repeat region substitutes for the loop of the hairpin structure and 8 nucleotides of the stem of the hairpin structure. In some embodiments, the internal linker in the repeat-anti-repeat region substitutes for the loop of the hairpin structure and 10 nucleotides of the stem of the hairpin structure.

In some embodiments, the internal linker in the repeat-anti-repeat region substitutes for the loop of the hairpin structure and 1, 2, 3, 4, 5, 6, 7, 8, 9, 10, 11, or 12 base pairs of the stem of the hairpin structure. In some embodiments, the internal linker in the repeat-anti-repeat region substitutes for the loop of the hairpin structure and 1, 2, 3, 4, 5, 6, 7, or 8 base pairs of the stem of the hairpin structure. In some embodiments, the internal linker in the repeat-anti-repeat region substitutes for the loop of the hairpin structure and 1, 2, 3, or 4 base pairs of the stem of the hairpin structure. In some embodiments, the internal linker in the repeat-anti-repeat region substitutes for the loop of the hairpin structure and 1 base pair of the stem of the hairpin structure. In some embodiments, the internal linker in the repeat-anti-repeat region substitutes for the loop of the hairpin structure and 2 base pairs of the stem of the hairpin structure. In some embodiments, the internal linker in the repeat-anti-repeat region substitutes for the loop of the hairpin structure and 3 base pairs of the stem of the hairpin structure. In some embodiments, the internal linker in the repeat-anti-repeat region substitutes for the loop of the hairpin structure and 4 base pairs of the stem of the hairpin structure. In some embodiments, the internal linker in the repeat-anti-repeat region substitutes for the loop of the hairpin structure and 5 base pairs of the stem of the hairpin structure. In some embodiments, the internal linker in the repeat-anti-repeat region substitutes for the loop of the hairpin structure and 6 base pairs of the stem of the hairpin structure. In some embodiments, the internal linker in the repeat-anti-repeat region substitutes for the loop of the hairpin structure and 7 base pairs of the stem of the hairpin structure.

In some embodiments, the internal linker in the repeat-anti-repeat region substitutes for all of the nucleotides constituting the loop of the hairpin structure.

In some embodiments, the internal linker in the repeat-anti-repeat region substitutes for all of the nucleotides constituting the loop and the upper stem of the hairpin structure.

Nexus Region

In some embodiment, the internal linker substitutes for 1 or 2 nucleotides of the loop of the nexus region of the gRNA. In some embodiment, the internal linker has a bridging length of about 6 to 18 atoms. In some embodiment, the internal linker has a bridging length of about 6-12 atoms.

Hairpin Region

In some embodiments, the internal linker substitutes for a hairpin structure in the hairpin region of the gRNA.

In some embodiments, the hairpin region is equivalent to a hairpin region obtainable by substituting an internal linker for 2, 3, 4, 5, 6, 7, 8, 9, 10, 11, 12, 13, 14, 15, 16, 17, 18, 19, 20, 21, or 22 nucleotides of a hairpin structure of a gRNA, e.g., any of the gRNAs shown in Table TA or any of SEQ ID NOs: 200-210 and 500-501.

In some embodiments, the internal linker substitutes for 2, 3, 4, 5, 6, 7, 8, 9, 10, 11, 12, 13, 14, 15, 16, 17, 18, 19, 20, 21, or 22 nucleotides of the hairpin structure. In some embodiments, the internal linker substitutes for 2-22 nucleotides of the hairpin structure. In some embodiments, the internal linker substitutes for 2-12 nucleotides of the hairpin structure. In some embodiments, the internal linker substitutes for 2-6 nucleotides of the hairpin structure. In some embodiments, the internal linker substitutes for 2-4 nucleotides of the hairpin structure. The gRNA comprising an internal linker in the hairpin region may form a duplex portion in the hairpin region. The internal linker in the hairpin region may substitute for the loop and the gRNA may form a duplex portion in the hairpin region. The internal linker in the hairpin region may substitute for the loop and one or more base pairs in the stem region and the gRNA may form a duplex portion in the hairpin region.

In some embodiments, the internal linker substitutes for a loop, or part thereof, of the hairpin structure in the hairpin region. In some embodiments, the internal linker substitutes for the loop and the stem, or part thereof, of the hairpin structure in the hairpin region.

In some embodiments, the internal linker substitutes for 2, 3, 4, or 5 nucleotides of the loop of the hairpin structure. In some embodiments, the internal linker substitutes for 2 nucleotides of the loop of the hairpin structure. In some embodiments, the internal linker substitutes for 3 nucleotides of the loop of the hairpin structure. In some embodiments, the internal linker substitutes for 4 nucleotides of the loop of the hairpin structure. In some embodiments, the internal linker substitutes for 5 nucleotides of the loop of the hairpin structure. In some embodiments, the internal linker substitutes for 2-5 nucleotides of the loop of the hairpin structure.

In some embodiments, the internal linker substitutes for the loop of the hairpin structure and at least 1 nucleotide of the stem of the hairpin structure. In some embodiments, the internal linker substitutes for the loop of the hairpin structure and 1, 2, 3, 4, 5, 6, 7, or 8 nucleotides of the stem of the hairpin structure. In some embodiments, the internal linker substitutes for the loop of the hairpin structure and at least 2 nucleotides of the stem of the hairpin structure.

In some embodiments, the internal linker substitutes for the loop of the hairpin structure and 2, 4, 6, 8, 10, 12, 14, 16, 18, 20, 22, or 24 nucleotides of the stem of the hairpin structure. In some embodiments, the internal linker substitutes for the loop of the hairpin structure and 2, 4, 6, 8, 10, 12, or 14 nucleotides of the stem of the hairpin structure. In some embodiments, the internal linker substitutes for the loop of the hairpin structure and 2, 4, 6, or 8 nucleotides of the stem of the hairpin structure. In some embodiments, the internal linker substitutes for the loop of the hairpin and 2 nucleotides of the stem of the hairpin structure. In some embodiments, the internal linker substitutes for the loop of the hairpin structure and 4 nucleotides of the stem of the hairpin structure. In some embodiments, the internal linker substitutes for the loop of the hairpin structure and 6 nucleotides of the stem of the hairpin structure. In some embodiments, the internal linker substitutes for the loop of the hairpin structure and 8 nucleotides of the stem of the hairpin structure. In some embodiments, the internal linker substitutes for the loop of the hairpin structure and up to 24 nucleotides of the stem of the hairpin structure.

In some embodiments, the internal linker substitutes for the loop of the hairpin structure and 1, 2, 3, 4, 5, 6, 7, 8, 9, 10, 11, or 12 base pairs of the stem of the hairpin structure. In some embodiments, the internal linker substitutes for the loop of the hairpin structure and 1, 2, 3, 4, 5, or 6 base pairs of the stem of the hairpin structure. In some embodiments, the internal linker substitutes for the loop of the hairpin structure and 1, 2, 3, or 4 base pairs of the stem of the hairpin structure. In some embodiments, the internal linker substitutes for the loop of the hairpin structure and 1 base pair of the stem of the hairpin structure. In some embodiments, the internal linker substitutes for the loop of the hairpin structure and 2 base pairs of the stem of the hairpin structure. In some embodiments, the internal linker substitutes for the loop of the hairpin structure and 3 base pairs of the stem of the hairpin structure. In some embodiments, the internal linker substitutes for the loop of the hairpin structure and 4 base pairs of the stem of the hairpin structure.

In some embodiments, the internal linker substitutes for all of the nucleotides constituting the loop of the hairpin structure.

In some embodiments, the internal linker substitutes for all of the nucleotides constituting the loop and the stem of the hairpin structure.

In some embodiments, the hairpin is a hairpin 1, and the internal linker substitutes for the hairpin 1. In some embodiments, the gRNA is a SpyCas9 gRNA and the internal linker substitutes for hairpin 1.

In further embodiments, the gRNA further comprises a hairpin 2 at 3′ to the hairpin 1. In some embodiments, the internal linker substitute for at least 2 nucleotides of a loop of the hairpin 2.

In some embodiments, hairpin 2 does not include any internal linker substitutions. In some embodiments, the gRNA is a Spy Cas9 gRNA and the hairpin 2 does not include any internal linker substitutions.

In some embodiments, the gRNA further comprises a guide region. In further embodiments, the guide region is 17, 18, 19, 20, or 21 nucleotides in length. In some embodiments, the gRNA does not comprise a guide region.

In some embodiments, the gRNA is a single guide RNA (sgRNA).

In some embodiments, the gRNA comprises a tracrRNA (trRNA).

B. Internal Linkers Structures—Physical Properties, Chemical Properties

gRNAs disclosed herein comprise an internal linker. In general, any internal linker compatible with the function of the gRNA may be used. It may be desirable for the linker to have a degree of flexibility. In some embodiments, the internal linker comprises at least two, three, four, five, six, or more on-pathway single bonds. A bond is on-pathway if it is part of the shortest path of bonds between the two nucleotides whose 5′ and 3′ positions are connected to the linker.

In some embodiments, the internal linker has a bridging length of about 6-40 Angstroms. In some embodiments, the internal linker has a bridging length of about 8-25 Angstroms. In some embodiments, the internal linker has a bridging length of about 8-15 Angstroms. In some embodiments, the internal linker has a bridging length of about 10-40 Angstroms. In some embodiments, the internal linker has a bridging length of about 10-35 Angstroms. In some embodiments, the internal linker has a bridging length of about 10-30 Angstroms. In some embodiments, the internal linker has a bridging length of about 10-25 Angstroms. In some embodiments, the internal linker has a bridging length of about 15-40 Angstroms. In some embodiments, the internal linker has a bridging length of about 15-35 Angstroms. In some embodiments, the internal linker has a bridging length of about 15-25 Angstroms. The length of the linker may in some embodiments be chosen based at least in part on the number of nucleotides for which the linker substitutes relative to a counterpart gRNA not containing an internal linker. For example, if the linker takes the place of two nucleotides, a linker having a length of about 8-15 Angstroms may be used, such as any of the embodiments described elsewhere herein encompassed within the range of about 8-15 Angstroms. If the linker takes the place of more than two nucleotides, a linker having a length of about 10-25 Angstroms may be used, such as any of the embodiments described elsewhere herein encompassed within the range of about 10-25 Angstroms.

Exemplary predicted linker lengths by number of atoms, number of ethylene glycol units, approximate linker length in Angstroms on the assumption that an ethylene glycol monomer is about 3.7 Angstroms, and suitable location for substitution of at least the entire loop portion of a hairpin structure are provided in the table below. Substitution of two nucleotides requires a linker length of at least about 11 Angstroms. Substitution of at least 3 nucleotides requires a linker length of at least about 16 Angstroms.

TABLE 1

| | Number of | Approximate | |
|---|---|---|---|
| Number | Ethylene | length in | Suitable location for complete |
| of atoms | Glycol units | Angstroms | loop substitution |
| 3 | 1 | 3.7 | Repeat-anti-repeat (for both loop |
| | | | and stem when no stem present) |
| 6 | 2 | 7.4 | Repeat-anti-repeat (for both loop |
| | | | and stem when no stem present) |
| 9 | 3 | 11.1 | Repeat-anti-repeat (for both loop |
| | | | and stem when no stem present), |
| | | | Nexus |
| 12 | 4 | 14.8 | Nexus |
| 15 | 5 | 18.5 | Repeat-anti-repeat, hairpin 1, |
| | | | hairpin 2 |
| 18 | 6 | 22.2 | Repeat-anti-repeat, hairpin 1, |
| | | | hairpin 2 |
| 21 | 7 | 25.9 | Repeat-anti-repeat, hairpin 1, |
| | | | hairpin 2 |
| 24 | 8 | 29.6 | Repeat-anti-repeat, hairpin 1, |
| | | | hairpin 2 |
| 27 | 9 | 33.3 | Repeat-anti-repeat, hairpin 1, |
| | | | hairpin 2 |
| 30 | 10 | 37 | Repeat-anti-repeat, hairpin 1, |
| | | | hairpin 2 |

In some embodiments, the internal linker comprises a structure of formula (I):

˜-L0-L1-L2-# (I)
wherein:
˜ indicates a bond to a 3′ substituent of the preceding nucleotide;
#indicates a bond to a 5′ substituent of the following nucleotide;
L0 is null or C1-3 aliphatic; L1 is -[E¹-(R¹)]m-, where
each R¹ is independently a C1-5 aliphatic group, optionally substituted with 1 or 2 E²,
each E¹ and E² are independently a hydrogen bond acceptor, or are each independently chosen from cyclic hydrocarbons, and heterocyclic hydrocarbons, and each m is 1, 2, 3, 4, 5, 6, 7, 8, 9, or 10; and
L2 is null, C1-3 aliphatic, or is a hydrogen bond acceptor.

In some embodiments, L1 comprises one or more —CH₂CH₂O—, —CH₂OCH₂—, or —OCH₂CH₂— units (“ethylene glycol subunits”). In some embodiments, the number of —CH₂CH₂O—, —CH₂OCH₂—, or —OCH₂CH₂— units is in the range of 1, 2, 3, 4, 5, 6, 7, 8, 9 or 10.

In some embodiments, m is 1, 2, 3, 4 or 5. In some embodiments, m is 1, 2, or 3. In some embodiments, m is 6, 7, 8, 9, or 10.

In some embodiments, L0 is null. In some embodiments, L0 is —CH₂— or —CH₂CH₂—.

In some embodiments, L2 is null. In some embodiments, L2 is —O—, —S—, or C1-3 aliphatic. In some embodiments, L2 is —O—. In some embodiments, L2 is —S—. In some embodiments, L2 is —CH₂— or —CH₂CH₂—.

The identities and values of the moieties and variables in Formula I may be chosen to provide an internal linker having any of the bridging lengths described herein. In some embodiments, the number of atoms in the shortest chain of atoms on the pathway from ˜ to #in the structure of Formula (I) is 30 or less, or 27 or less, or 24 or less, or 21 or less, or is 18 or less, or is 15 or less, or is 12 or less, or is 10 or less.

In some embodiments, the number of atoms in the shortest chain of atoms on the pathway from ˜ to #in the structure of Formula (I) is from 6 to 30, or is from 9 to 30, or is from 9 to 21. In some embodiments, the number of atoms in the shortest chain of atoms on the pathway from ˜ to #in the structure of Formula (I) is 9. In some embodiments, the number of atoms in the shortest chain of atoms on the pathway from ˜ to #in the structure of Formula (I) is 18.

In some embodiments, each C₁₋₃ aliphatic group and C₁₋₅ aliphatic group is saturated. In some embodiments, at least one C₁₋₅ aliphatic group is a C₁₋₄ alkylene, or wherein at least two C₁₋₅ aliphatic groups are a C₁₋₄ alkylene, or wherein at least three C₁₋₅ aliphatic groups are a C₁₋₄ alkylene. In some embodiments, at least one R¹ is selected from —CH₂—, —CH₂CH₂—, —CH₂CH₂CH₂—, or —CH₂CH₂CH₂CH₂—. In some embodiments, each R¹ is independently selected from —CH₂—, —CH₂CH₂—, —CH₂CH₂CH₂—, or —CH₂CH₂CH₂CH₂—. In some embodiments, each R¹ is —CH₂CH₂—.

In some embodiments, at least one C₁₋₅ aliphatic group is a C₁₋₄ alkenylene, or wherein at least two C₁₋₅ aliphatic groups are a C₁₋₄ alkenylene, or wherein at least three C₁₋₅ aliphatic groups are a C₁₋₄ alkenylene. In some embodiments, at least one R¹ is selected from —CHCH—, —CHCHCH₂—, or —CH₂CHCHCH₂—.

In some embodiments, each E¹ is independently chosen from —O—, —S—, —NH—, —NR—, —C(O)—O—, —OC(O)O—, —C(O)—NR—, —OC(O)—NR—, —NC(O)—NR—, —P(O)₂O—, —OP(O)₂O—, —OP(R)(O)O—, —OP(O)(S)O—, —S(O)₂— and cyclic hydrocarbons, and heterocyclic hydrocarbons. In some embodiments, each E¹ is independently chosen from —O—, —S—, —NH—, —NR—, —C(O)—O—, —OC(O)O—, —P(O)₂O—, —OP(O)₂O—, and —OP(R)(O)O.

In some embodiments, each E¹ is —O—.

In some embodiments, each E¹ is —S—.

In some embodiments, at least one C₁₋₅ aliphatic group in R¹ is optionally substituted with one E².

In some embodiments, each E² is independently chosen from —OH, —OR, —ROR, —SH, —SR, —C(O)—R, —C(O)—OR, —OC(O)—OR, —C(O)—H, —C(O)—OH, —OPO₃, —PO₃, —RPO₃, —S(O)₂—R, —S(O)₂—OR, —RS(O)₂—R, —RS(O)₂—OR, —SO₃, and cyclic hydrocarbons, and heterocyclic hydrocarbons. In some embodiments, each E² is independently chosen from —OH, —OR, —SH, —SR, —C(O)—R, —C(O)—OR, —OC(O)—OR, —OPO₃, —PO₃, —RPO₃, and —SO₃.

In some embodiments, each E² is —OH or —OR.

In some embodiments, each E² is —SH or —SR.

In some embodiments, the internal linker comprises at least two, three, four, five, or six ethylene glycol subunits covalently linked to each other. In some embodiments, the internal linker comprises a linker having from 1 to 10 ethylene glycol units. In some embodiments, the internal linker comprises a linker having from 2 to 7 ethylene glycol units. In some embodiments, the internal linker comprises a linker having from 3 to 6 ethylene glycol units. In some embodiments, the internal linker comprises a linker having 3 ethylene glycol units. In some embodiments, the internal linker comprises a linker having 6 ethylene glycol units.

In some embodiments, the internal linker comprises a PEG-linker. In some embodiments, the internal linker comprises a PEG-linker having from 1 to 9 ethylene glycol units. In some embodiments, the internal linker comprises a PEG-linker having from 3 to 6 ethylene glycol units. In some embodiments, the internal linker comprises a PEG-linker having 3 ethylene glycol units. In some embodiments, the internal linker comprises a PEG-linker having 6 ethylene glycol units.

In some embodiments, the internal linker has a bridging length of about 9-30 atoms, optionally about 15-21 atoms, and the linker substitutes for at least 4 nucleotides of the gRNA. For brevity, an internal linker having a bridging length of about 15-21 atoms is referred to elsewhere herein as a “linker 1.” The internal linker having a bridging length of about 9-30 atoms, optionally about 15-21 atoms may be chosen from any such embodiment described herein. The internal linker having a bridging length of about 9-30 atoms, optionally about 15-21 atoms may have any compatible feature described herein for internal linkers.

In some embodiments, a linker comprises a plurality of polyethylene glycol subunits, such as at least 2, 3, 4, 5, 6, 7, 8, 9, or 10 polyethylene glycol subunits. In some embodiments, a linker comprises at least 5, 6, or 7 polyethylene glycol subunits. In some embodiments, a linker consists of at least 5, 6, or 7 polyethylene glycol subunits.

In some embodiments, the internal linker has a bridging length of about 6-18 atoms, optionally about 6-12 atoms, and the linker substitutes for at least 2 nucleotides of the gRNA. For brevity, an internal linker having a bridging length of about 6-18 atoms, optionally about 6-12 atoms is referred to elsewhere herein as a “linker 2.” The internal linker having a bridging length of about 6-18 atoms, optionally about 6-12 atoms may be chosen from any such embodiment described herein. The internal linker having a bridging length of about 6-12 atoms may have any compatible feature described herein for internal linkers. In some embodiments, a linker 2 comprises a plurality of polyethylene glycol (PEG) subunits, such as at least 2, 3, or 4 polyethylene glycol subunits. In some embodiments, a linker 1 comprises at least 2, 3, or 4 polyethylene glycol subunits. In some embodiments, a linker 1 consists of at least 2, 3, or 4 polyethylene glycol subunits.

Exemplary PEG containing linkers include the following:

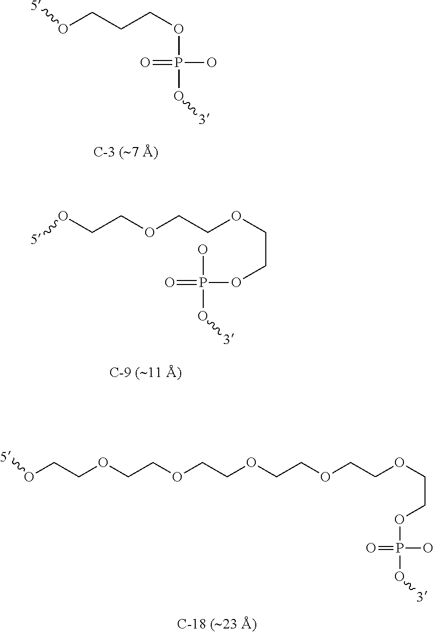

Linkers for use in the compositions and methods provided herein are known in the art and commercially available from various sources including, but are not limited to, Biosearch Technologies (e.g., Spacer-CE Phosphoramidite C2, 2-(4,4′-Dimethoxytrityloxy)ethyl-1-[(2-cyanoethyl)-(N,N-diisopropyl)]-phosphoramidite and C6 Spacer Amidite (DMT-1,6-Hexandiol)); Glen Research (Spacer Phosphoramidite C3, 3-(4,4′-Dimethoxytrityloxy)propyl-1-[(2-cyanoethyl)-(N,N-diisopropyl)]-phosphoramidite; Spacer Phosphoramidite 9, 9-O-Dimethoxytrityl-triethylene glycol,1-[(2-cyanoethyl)-(N,N-diisopropyl)]-phosphoramidite; Spacer C12 CE Phosphoramidite, 12-(4,4′-Dimethoxytrityloxy)dodecyl-1-[(2-cyanoethyl)-(N,N-diisopropyl)]-phosphoramidite; and Spacer Phosphoramidite 18, 18-O-Dimethoxytritylhexaethyleneglycol,1-[(2-cyanoethyl)-(N,N-diisopropyl)]-phosphoramidite).

C. Methods of Making

Methods of synthesizing a gRNA comprising an internal linker disclosed herein are provided. Suitable precursors, e.g., linker can be introduced into an sgRNA oligonucleotide by using the corresponding phosphoramidite building block in methods of making sgRNA in a single synthetic process. Such building blocks are commercially available or can be prepared by known methods.

Methods of synthesis include a series of sequential coupling reactions including covalently linking a first nucleotide to a second nucleotide; covalently linking an internal linker to a second nucleotide; and covalently linking a third nucleotide to the internal linker. In certain embodiments, such linkages are performed using phosphoramidite chemistry. In certain embodiments, the method includes covalent linkage of a second linker to the first linker prior to covalent linkage of the third nucleotide.

In some embodiments, a solid support covalently attached to the linker of the gRNA disclosed herein is provided.

The gRNA provided herein with internal linkers are made in a single synthetic process such that a full-length gRNA strand (sgRNA, crRNA, or trRNA) is produced by the synthetic method. In the case of a dgRNA, the crRNA and trRNA are synthesized separately and annealed. That is, when the gRNA is made as a dgRNA, the separately synthesized portions do not require covalent linkage to form a stable gRNA. In certain embodiments, the crRNA and trRNA of a dgRNA containing an internal linker as provided herein, does not include a covalent linkage between the crRNA and the trRNA.

In preferred embodiments, the gRNA is not made using click chemistry.

D. Types of Guide RNAs

In some embodiments, the guide RNA is a single guide RNA.

In some embodiments, the guide RNA comprises a tracrRNA (trRNA).

Sequences of exemplary gRNAs are shown in Table 1A below. In some embodiments, the guide RNA comprises a nucleic acid sequence of any one of SEQ ID NOs: 200-210 and 500-501 wherein an internal linker substitutes for one or more nucleotides. In some embodiments, at least one nucleotide shown in bold in Table 1A is replaced with an internal linker. In some embodiments, at least two consecutive nucleotides shown in bold in Table 1 are replaced with an internal linker. In some embodiments, at least three consecutive nucleotides shown in bold in Table 1A are replaced with an internal linker. In some embodiments, at least four consecutive nucleotides shown in bold in Table 1A are replaced with an internal linker. In some embodiments, at least two nonconsecutive nucleotides shown in bold in Table 1A are replaced with an internal linker. In some embodiments, at least a first two or more consecutive nucleotides and at least a second two or more consecutive nucleotides shown in bold in Table 1A are replaced with an internal linker, wherein the first two or more consecutive nucleotides are not consecutive with the second two or more consecutive nucleotides. In some embodiments, at least a first three or more consecutive nucleotides and at least a second three or more consecutive nucleotides shown in bold in Table 1A are replaced with an internal linker, wherein the first three or more consecutive nucleotides are not consecutive with the second three or more consecutive nucleotides. In some embodiments, at least a first four or more consecutive nucleotides and at least a second two or more consecutive nucleotides shown in bold in Table 1A are replaced with an internal linker, wherein the first four or more consecutive nucleotides are not consecutive with the second two or more consecutive nucleotides. In some embodiments, at least a first four or more consecutive nucleotides and at least a second four or more consecutive nucleotides shown in bold in Table 1A are replaced with an internal linker, wherein the first four or more consecutive nucleotides are not consecutive with the second four or more consecutive nucleotides.

TABLE 1A
Table of exemplary guide RNAs
(as used herein, “Linker 1” refers to an
internal linker having a bridging length
of about 15-21 atoms. As used herein,
“Linker 2” refers to an internal linker having
a bridging length of about 6-12 atoms.)

| | | | gRNA sequence (Exemplary nucleotides subject to | |
|---|---|---|---|---|
| | | SEQ | replacement with internal linkers in bold. In some | |
| | Length | ID | embodiments, bold italics indicate Linker 1 and bold | # of |
| Cas type | (nt) | NO: | roman (not italic) indicates Linker 2 replacements.) | linkers |
| SpyCas9¹⁻⁴ | 100 | 200 | NNNNNNNNNNNNNNNNNNNNGUUUUAGAGCUAGAAAUAGCAAGUUAAAAUAAGGC | 3 |
| | | | UAGUCCGUUAUCAACUUGAAAAAGUGGCACCGAGUCGGUGCUUUU | |
| SpyCas9¹⁻⁴ | 90 | 201 | NNNNNNNNNNNNNNNNNNNNGUUUUAGAGCUAGAAAUAGCAAGUUAAAAUAAGGC | 3 |
| | | | UAGUCCGUUAUCACGAAAGGGCACCGAGUCGGUGC | |
| SauCas9⁵ | 100 | 202 | NNNNNNNNNNNNNNNNNNNNGUUUUAGUACUCUGGAAACAGAAUCUACUAAAACA | 3 |
| | | | AGGCAAAAUGCCGUGUUUAUCUCGUCAACUUGUUGGCGAGAUUUU | |
| CdiCas9⁶ | 113 | 203 | NNNNNNNNNNNNNNNNNNNNACUGGGGUUCAGGAAACUGAACCUCAGUAAGCAUU | 3 |
| | | | GGCUCGUUUCCAAUGUUGAUUGCUCCGCCGGUGCUCCUUAUUUUUAAGGGCGCCG | |
| | | | GCA | |
| St1Cas9⁷ | 117 | 204 | NNNNNNNNNNNNNNNNNNNNGUUUUUGUACUCUCAAGAUUCAAUAAUCUUGCAGA | 2 |
| | | | AGCUACAAAGAUAAGGCUUCAUGCCGAAAUCAACACCCUGUCAUUUUAUGGCAGG | |
| | | | GUGUUUU | |
| SthCas9⁸ | 97 | 205 | NNNNNNNNNNNNNNNNNNNNGUUUUUGUACUCGAAAGAAGCUACAAAGAUAAGGC | 3 |
| | | | UUCAUGCCGAAAUCAACACCCUGUCAUUUUAUGGCAGGGUGU | |
| AceCas9 | 94 | 206 | NNNNNNNNNNNNNNNNNNNNGCUGGGGAGCCUGAAAAGGCUACCUAGCAAGACCC | 3 |
| | | | CUUCGUGGGGUCGCAUUCUUCACCCCCAGCAGGGGGUUC | |
| CjeCas9⁹ | 93 | 207 | NNNNNNNNNNNNNNNNNNNNGUUUUAGUCCCUGAAAAGGGACUAAAAUAAAGAGU | 1 |
| | | | UUGCGGGACUCUGCGGGGUUACAAUCCCCUAAAACCGC | |
| FnoCas9¹⁰ | 94 | 208 | NNNNNNNNNNNNNNNNNNNNNGUUUCAGUUGCGCCGAAAGGCGCUCUGUAAUCAU | 1 |
| | | | UAAAAAGUAUUUUGAACGGACCUCUGUUUGACACGUCUG | |
| AsCpf1/ | 45 | 209 | UAAUUUCUACUCUUGUAGAUNNNNNNNNNNNNNNNNNNNNNNNNN | 1 |
| Cas12a¹¹ | | | | |
| EsCas13d¹² | 52 | 210 | CUGGUGCAAAUUUGCACUAGCUUAAAACNNNNNNNNNNNNNNNNNNNNNNNNNNN | 1 |
| NmeCas9¹³ | 101- | 500 | NNNNNNNNNNNNNNNNNNNNNNNNGUUGUAGCUCCCUUUCUCAUUUCGGAAACGA | 3 |
| | 145 | | AAUGAGAACCGUUGCUACAAUAAGGCCGUCUGAAAAGAUGUGCCGCAACGCUCUGC | |
| | | | CCCUUAAAGCUUCUGCUUUAAGGGGCAUCGUUUA | |
| | | | (underlined indicates the nucleotides that can be deleted) | |
| Shortened | 101 | 501 | NNNNNNNNNNNNNNNNNNNNNNNNGUUGUAGCUCCCUUCGAAAGACCGUUGCUAC | 3 |
| NmeCas9 | | | AAUAAGGCCGUCGAAAGAUGUGCCGCAACGCUCUGCCUUCUGGCAUCGUU | |

References for the guide RNA for different species of Cas9:
1. Science. 2015 Jun. 26;348(6242):1477-81. doi: 10.1126/science.aab1452. PMID: 26113724.
2. Nature. 2014 Sep. 25;513(7519):569-73. doi: 10.1038/nature13579. Epub 2014 Jul. 27. PMID: 25079318; PMCID: PMC4176945.
3. Nat Struct Mol Biol. 2019 Aug. 26(8):679-685. doi: 10.1038/s41594-019-0258-2. Epub 2019 Jul. 8. PMID: 31285607; PMCID: PMC6842131.
4. Cell. 2014 Feb. 27;156(5):935-49. doi: 10.1016/j.cell.2014.02.001. Epub 2014 Feb. 13. PMID: 24529477; PMCID: PMC4139937.
5. Cell. 2015 Aug. 27;162(5):1113-26. doi: 10.1016/j.cell.2015.08.007. PMID: 26317473; PMCID: PMC4670267.
6. Nat Commun. 2019 Apr. 29; 10(1):1968. doi: 10.1038/s41467-019-09741-6. PMID: 31036811; PMCID: PMC6488586.
7. Mol Cell. 2019 Dec. 19;76(6):922-937.e7. doi: 10.1016/j.molcel.2019.09.012. Epub 2019 Oct. 8. PMID: 31604602.
8. Nat Catal 3, 813-823 (2020). https://doi.org/10.1038/s41929-020-00506-9
9. Mol Cell. 2017 Mar. 16;65(6):1109-1121.e3. doi: 10.1016/j.molcel.2017.02.007. PMID: 28306506.
10. Cell. 2016 Feb. 25;164(5):950-61. doi: 10.1016/j.cell.2016.01.039. Epub 2016 Feb. 11. PMID: 26875867; PMCID: PMC4899972.
11. Cell Res. 2016 Aug. 26(8):901-13. doi: 10.1038/cr.2016.88. Epub 2016 Jul. 22. PMID: 27444870; PMCID: PMC4973337.
12. Biotech. Bioeng. 2021 Apr. 30; DOI: 10.1002/bit.27813; PMID: 33964175
13. Mol Cell. 2019 Dec. 19;76(6):938-952.e5. doi: 10.1016/j.molcel.2019.09.025. Epub 2019 Oct. 24. PMID: 31668930 PMCID: PMC6934045 DOI: 10.1016/j.molcel.2019.09.025

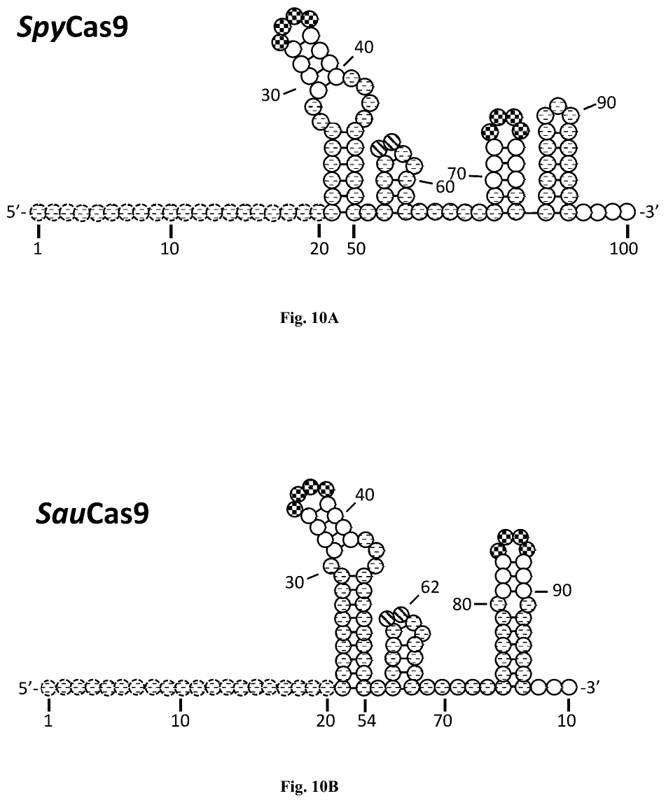

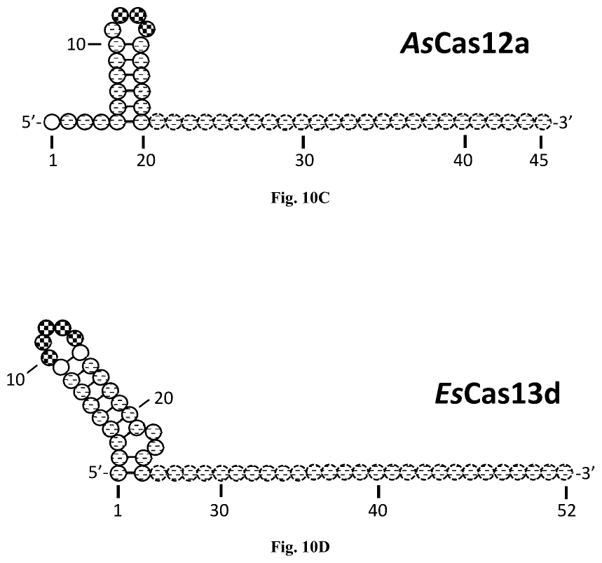

In some embodiments, the guide RNA comprises a nucleic acid sequence of any one of SEQ ID NOs: 200-210 and 500, including modifications disclosed elsewhere herein. Exemplary sgRNAs are shown in FIG. 10A-10E in which the guide region (target-binding region), and the nucleotides that can be substituted for the internal linkers are shown. Table TB shows various embodiments of the gRNA structures and species with possible number of internal linkers and positions.

TABLE 1B

| | | # | |
|---|---|---|---|
| gRNA | | internal | |
| structures | Type | linkers | Positions of internal linkers |
| Repeat/anti- | Spy | 3 | Repeat/Anti-Repeat region; nexus |
| R; nexus; | | | (within hairpin or replace hairpin), |
| Hp1; Hp2 | | | Hairpin 1 (Hp1) |
| Repeat/anti- | Spy | 2 | Any two of Repeat/Anti-R; nexus |
| R; nexus; | | | (within hairpin or replace hairpin), |
| Hp1; Hp2 | | | Hp1 |
| Repeat/anti- | Spy | 1 | Any of Repeat/Anti-R; nexus |
| R; nexus; | | | (within hairpin or replace hairpin), |
| Hp1; Hp2 | | | Hp1 |
| Repeat/anti- | Cdi, Sau, | 3 | All of repeat/anti-R; Hp1; Hp2 |
| R; Hp1; | Sth, and Ace | | |
| Hp2 | | | |
| Repeat/anti- | Cdi, Sau, | 2 | For Sau, preferred not Hp2 |
| R; Hp1; | Sth, and Ace | | |
| Hp2 | | | |
| Repeat/anti- | Cdi, Sau, | 1 | For Sau, preferred not Hp2 |
| R; Hp1; | Sth, and Ace | | |
| Hp2 | | | |
| Repeat/anti- | St1 | 2 | Repeat/anti-R; Hp2 |
| R; Hp1; | | | |
| Hp2 | | | |
| Repeat/anti- | Cje | 1 | Repeat/anti-R |
| R; Hp1; | | | |
| Hp2 | | | |
| Repeat/anti- | Cpf1 - | 1 | Repeat/anti-R |
| R | various | | |
| Repeat/anti- | Nme | 3 | All of repeat/anti-R; Hp1; Hp2 |
| R; Hp1; | | | |
| Hp2 | | | |
| Repeat/anti- | Nme | 2 | Any two of repeat/anti-R; |
| R; Hp1; | | | Hp1; Hp2 |
| Hp2 | | | |
| Repeat/anti- | Nme | 1 | Any one of repeat/anti-R; |
| R; Hp1; | | | Hp1; Hp2 |
| Hp2 | | | |

a. SpyCas9 Guide RNAs

In some embodiments, the guide RNA is a S. pyogenes Cas9 (“SpyCas9”) guide RNA. As used herein, a SpyCas9 guide RNA mean that it is functional with SpyCas9. The same applies to other gRNAs for different species of Cas9 disclosed herein.

In some embodiments, the guide RNA comprises a nucleic acid sequence of SEQ ID NO: 200 or 201. In some embodiments, the guide RNA is a modified SpyCas9 guide RNA. In some embodiments, the guide RNA comprises a nucleic acid sequence of SEQ ID NO: 200 or 201, including modifications disclosed elsewhere herein.

In some embodiments, the sgRNA comprises a guide region and a conserved portion 3′ to the guide region, wherein the conserved portion comprises a repeat-anti-repeat region, a nexus region, a hairpin 1 region, and a hairpin 2 region, and comprises at least one of:
a first internal linker substituting for at least 2 nucleotides, optionally at least 4 nucleotides, of an upper stem region of the repeat-anti-repeat region;
a second internal linker substituting for 1 or 2 nucleotides of the nexus region; and
a third internal linker substituting for at least 2 nucleotides of the hairpin 1.

An exemplary SpyCas9 sgRNA is shown in FIG. 10A-in which the guide region (target-binding region), and the nucleotides that can be substituted for the first linker in the repeat-anti-repeat-region, the second linker in the nexus region, and the third linker in the hairpin 1 region.

In some embodiments, the sgRNA comprises the first internal linker and the second internal linker. In some embodiments, the sgRNA comprises the first internal linker and the third internal linker. In some embodiments, the sgRNA comprises the second internal linker and the second internal linker. In some embodiments, the sgRNA comprises the first internal linker, the second internal linker, and the third internal linker.

In some embodiments, the first internal linker has a bridging length of about 9-30 atoms, optionally about 15-21 atoms.

In some embodiments, the first internal linker substitutes for 2, 3, 4, 5, 6, 7, 8, 9, 10, 11, or 12 nucleotides of the upper stem region. In some embodiments, the first internal linker substitutes for a loop, or part thereof, of the upper stem region. In some embodiments, the first internal linker substitutes for the loop and the stem, or part thereof, of the upper stem region.

In some embodiments, the first internal linker substitutes for 2, 3, or 4 nucleotides of the loop of the upper stem region. In some embodiments, the first internal linker substitutes for 4 nucleotides of the loop of the upper stem region.

In some embodiments, the first internal linker substitutes for the loop of the upper stem region and at least 2, 4, 6, or 8 nucleotides of the stem of the upper stem region. In some embodiments, the first internal linker substitutes for the loop of the upper stem region and 1, 2, 3, or 4 base pairs of the stem of the upper stem region. In some embodiments, the first internal linker substitutes for the loop of the upper stem region and 1 base pair of the stem of the upper stem region. In some embodiments, the first internal linker substitutes for the loop of the upper stem region and 2 base pairs of the stem of the upper stem region. In some embodiments, the first internal linker substitutes for the loop of the upper stem region and 3 base pairs of the stem of the upper stem region. In some embodiments, the first internal linker substitutes for the loop of the upper stem region and 4 base pairs of the stem of the upper stem region.

In some embodiments, the first internal linker substitutes for all of the nucleotides constituting the loop of the upper stem region (i.e., the portion of the stem above the bulge). In some embodiments, the first internal linker substitutes for all of the nucleotides constituting the loop and the stem of the upper stem region.

In some embodiments, the bulge in the repeat-anti-repeat region does not contain a linker. In some embodiments, the lower stem portion of the repeat-anti-repeat region does not contain a linker.

In some embodiments, the second internal linker has a bridging length of about 6-18 atoms, optionally 9-18 atoms. In some embodiments, the second internal linker substitutes for 2 nucleotides of the nexus region of the sgRNA.

In some embodiments, the third internal linker has a bridging length of about 9-30 atoms, optionally 15-21 atoms.

In some embodiments, the third internal linker substitutes for 2, 4, 6, 8, or 10 nucleotides of the hairpin 1 of the gRNA. In some embodiments, the third linker substitutes for 1, 2, 3, 4, or 5 base pairs of the hairpin 1 of the gRNA. In some embodiments, the third linker substitutes for 1 base pair of the hairpin 1 of the gRNA. In some embodiments, the third linker substitutes for 2 base pairs of the hairpin 1 of the gRNA. In some embodiments, the third linker substitutes for 3 base pairs of the hairpin 1 of the gRNA. In some embodiments, the third linker substitutes for 4 base pairs of the hairpin 1 of the gRNA. In some embodiments, the third linker substitutes for 5 base pairs of the hairpin 1 of the gRNA.

In some embodiments, the third internal linker substitutes for a loop, or part thereof, of the hairpin 1. In some embodiments, the third internal linker substitutes for the loop and the stem, or part thereof, of the hairpin 1.

In some embodiments, the third internal linker substitutes for 2, 3, or 4 nucleotides of the loop of the hairpin 1. In some embodiments, the first internal linker substitutes for 2 nucleotides of the loop of the hairpin 1. In some embodiments, the first internal linker substitutes for 3 nucleotides of the loop of the hairpin 1. In some embodiments, the first internal linker substitutes for 4 nucleotides of the loop of the hairpin 1.

In some embodiments, the third internal linker substitutes for the loop of the hairpin and at least 1 nucleotide of the stem of the hairpin. In some embodiments, the third internal linker substitutes for the loop of the hairpin and 2, 4, or 6 nucleotides of the stem of the hairpin. In some embodiments, the third internal linker in the repeat-anti-repeat region substitutes for the loop of the hairpin and 1, 2, or 3 base pairs of the stem of the hairpin.

In some embodiments, the third internal linker substitutes for all of the nucleotides constituting the loop of the hairpin. In some embodiments, the third internal linker substitutes for all of the nucleotides constituting the loop and the stem of the hairpin.

In some embodiments, a hairpin 2 region of the sgRNA does not contain any internal linker.

In some embodiments, the second internal linker substitutes for 2 nucleotides of a loop of the nexus region of the sgRNA.

In some embodiments, the sgRNA comprises a conserved portion comprising a sequence of SEQ ID NO: 200. In some embodiments, 2, 3 or 4 of nucleotides 33-36 are substituted for the first internal linker relative SEQ ID NO: 200. In some embodiments, nucleotides 32-37 are substituted for the first internal linker relative SEQ ID NO: 200. In some embodiments, nucleotides 31-38 are substituted for the first internal linker relative SEQ ID NO: 200. In some embodiments, nucleotides 30-39 are substituted for the first internal linker relative SEQ ID NO: 200. In some embodiments, nucleotides 29-40 are substituted for the first internal linker relative SEQ ID NO: 200. In some embodiments, nucleotide 55-56 are substituted for the second internal linker relative SEQ ID NO: 200. In some embodiments, 2, 3, or 4 of nucleotides 73-76 are substituted for the third internal linker relative SEQ ID NO: 200. In some embodiments, nucleotides 72-77 are substituted for the third internal linker relative SEQ ID NO: 200. In some embodiments, nucleotides 71-78 are substituted for the third internal linker relative SEQ ID NO: 200. In some embodiments, nucleotides 70-79 are substituted for the third internal linker relative SEQ ID NO: 200. In some embodiments, nucleotides 97-100 are deleted relative SEQ ID NO: 200.

In some embodiments, the sgRNA comprises a sequence of SEQ ID NO: 201. In some embodiments, 2, 3 or 4 of nucleotides 33-36 are substituted for the first internal linker relative SEQ ID NO: 201. In some embodiments, nucleotides 32-37 are substituted for the first internal linker relative SEQ ID NO: 201. In some embodiments, nucleotides 31-38 are substituted for the first internal linker relative SEQ ID NO: 201. In some embodiments, nucleotides 30-39 are substituted for the first internal linker relative SEQ ID NO: 201. In some embodiments, nucleotides 29-40 are substituted for the first internal linker relative SEQ ID NO: 201. In some embodiments, nucleotide 55-56 are substituted for the second internal linker relative SEQ ID NO: 201. In some embodiments, 2, 3, or 4 of nucleotides 50-53 are substituted for the third internal linker relative SEQ ID NO: 201. In some embodiments, nucleotides 49-54 are substituted for the third internal linker relative SEQ ID NO: 201. In some embodiments, nucleotides 77-80 are deleted relative SEQ ID NO: 201.

b. Additional Guide RNAs

In some embodiments, the sgRNA is not from S. pyogenes Cas9 (“non-spyCas9”).

In some embodiments, the guide RNA is a Staphylococcus aureus Cas9 (“SauCas9”) guide RNA. An exemplary SauCas9 sgRNA is shown in FIG. 10B. In some embodiments, the guide RNA is a modified SauCas guide RNA.

In some embodiments, a sgRNA comprises a guide region and a conserved portion 3′ to the guide region, wherein conserved portion comprises a repeat-anti-repeat region, a hairpin 1 region, and a hairpin 2 region, and further comprises at least one of:
1) a first internal linker substituting for at least 2 nucleotides, optionally at least 4 nucleotides, of an upper stem region of the repeat-anti-repeat region of the sgRNA;
2) a second internal linker substituting for 1 or 2 nucleotides of the hairpin 1 of the sgRNA; or
3) a third internal linker substituting for at least 2 nucleotides, optionally at least 4 nucleotides, of the hairpin 2 of the sgRNA.

In some embodiments, the sgRNA comprises the first internal linker and the second internal linker. In some embodiments, the sgRNA comprises the first internal linker and the third internal linker. In some embodiments, the sgRNA comprises the second internal linker and the third internal linker. In some embodiments, the sgRNA comprises the first internal linker, the second internal linker, and the third internal linker.

In some embodiments, the first internal linker has a bridging length of about 9-30 atoms, optionally about 15-21 atoms. In some embodiments, the first internal linker is in a hairpin between a first portion of the sgRNA and a second portion of the sgRNA, wherein the first portion and the second portion together form a duplex portion. In some embodiments, the first internal linker substitutes for 2, 3, 4, 5, 6, 7, 8, 9, 10, 11, or 12 nucleotides of the upper stem region. In some embodiments, the first internal linker substitutes for a loop, or part thereof, of the upper stem region. In some embodiments, the first internal linker substitutes for the loop and the stem, or part thereof, of the upper stem region. In some embodiments, the first internal linker substitutes for 2, 3, or 4 nucleotides of the loop of the upper stem region.

In some embodiments, the first internal linker substitutes for the loop of the upper stem region and at least 2, 4, 6, or 8 nucleotides of the stem of the upper stem region. In some embodiments, the first internal linker substitutes for the loop of the upper stem region and 1, 2, 3, or 4 base pairs of the stem of the upper stem region. In some embodiments, the first internal linker substitutes for all of the nucleotides constituting the loop of the upper stem region.

In some embodiments, the second internal linker has a bridging length of about 9-18 atoms. In some embodiments, the second internal linker substitutes for 2 nucleotides of the hairpin 1 of the sgRNA. In some embodiments, the second internal linker substitutes for 2 nucleotides of a stem region of the nexus region of the sgRNA.

In some embodiments, the third internal linker has a bridging length of about 9-30 atoms, optionally about 15-21 atoms. In some embodiments, the third internal linker substitutes for 4, 5, 6, 7, 8, 9, 10, 11, or 12 nucleotides of the hairpin 2 of the gRNA. In some embodiments, the third linker substitutes for 1, 2, 3, 4, or 5 base pairs of the hairpin 2 of the gRNA. In some embodiments, the internal linker substitutes for 2-6 nucleotides of hairpin 2. In some embodiments, the internal linker substitutes for 2-4 nucleotides of hairpin 2.

In some embodiments, the third internal linker substitutes for a loop, or part thereof, of the hairpin 2. In some embodiments, the third internal linker substitutes for the loop and the stem, or part thereof, of the hairpin 2.

In some embodiments, the third internal linker substitutes for 2, 3, or 4 nucleotides of the loop of the hairpin 2. In some embodiments, the third internal linker substitutes for the loop of the hairpin and at least 1 nucleotide of the stem of the hairpin 2. In some embodiments, the third internal linker substitutes for the loop of the hairpin and 2, 3, 4, 5, or 6 nucleotides of the stem of the hairpin 2. In some embodiments, the third internal linker in the repeat-anti-repeat region substitutes for the loop of the hairpin and 1, 2, or 3 base pairs of the stem of the hairpin 2. In some embodiments, the third internal linker substitutes for all of the nucleotides constituting the loop of the hairpin 2. In some embodiments, the third internal linker is in a hairpin between a first portion of the sgRNA and a second portion of the sgRNA, wherein the first portion and the second portion together form a duplex portion.

In some embodiments, the guide RNA comprises a nucleic acid sequence of SEQ ID NO: 202. In some embodiments, the guide RNA comprises a nucleic acid sequence of SEQ ID NO: 202, including modifications disclosed elsewhere herein.

In some embodiments, 2, 3, or 4 of nucleotides 35-38 are substituted for the first internal linker relative SEQ ID NO: 202. In some embodiments, nucleotides 34-39 are substituted for the first internal linker relative SEQ ID NO: 202. In some embodiments, nucleotides 33-40 are substituted for the first internal linker relative SEQ ID NO: 202. In some embodiments, nucleotides 32-41 are substituted for the first internal linker relative SEQ ID NO: 202. In some embodiments, nucleotides 31-42 are substituted for the first internal linker relative SEQ ID NO: 202. In some embodiments, nucleotide 61-62 are substituted for the second internal linker relative SEQ ID NO: 202. In some embodiments, 2, 3, or 4 of nucleotides 84-87 are substituted for the third internal linker relative SEQ ID NO: 202. In some embodiments, nucleotides 83-88 are substituted for the third internal linker relative SEQ ID NO: 202. In some embodiments, nucleotides 82-89 are substituted for the third internal linker relative SEQ ID NO: 202. In some embodiments, nucleotides 81-90 are substituted for the third internal linker relative SEQ ID NO: 202. In some embodiments, nucleotides 97-100 are deleted relative SEQ ID NO: 202.

In some embodiments, wherein the gRNA is a SauCas9 guide RNA, and does not include the third internal linker.

In some embodiments, the guide RNA is a Corynebacterium diphtheriae Cas9 (“CdiCas9”) guide RNA. In some embodiments, the guide RNA is a modified CdiCas9 guide RNA. In some embodiments, the guide RNA comprises a nucleic acid sequence of SEQ ID NO: 203. In some embodiments, the guide RNA comprises a nucleic acid sequence of SEQ ID NO: 203, including modifications disclosed elsewhere herein.

In some embodiments, the gRNA is a C. diphtheriae Cas9 (CdiCas9) guide RNA, an S. thermophilus Cas9 (SthCas9) guide RNA, or an Acidothermus cellulolyticus Cas9 (AceCas9) guide RNA.

In some embodiments, the guide RNA is a Streptococcus thermophilus Cas9 (“St1Cas9” or “SthCas9”) guide RNA. In some embodiments, the guide RNA is a modified St1Cas9 guide RNA. In some embodiments, the guide RNA comprises a nucleic acid sequence of SEQ ID NO: 204 or 205. In some embodiments, the guide RNA comprises a nucleic acid sequence of SEQ ID NO: 204 or 205, including modifications disclosed elsewhere herein.

In some embodiments, a sgRNA comprises a guide region and a conserved portion 3′ to the guide region, wherein the conserved portion comprises a repeat-anti-repeat region, a hairpin 1 region, and a hairpin 2 region, and comprises a first internal linker substituting for at least 4 nucleotides of the repeat-anti-repeat region and a second internal linker substituting for at least 3 nucleotides of the hairpin 2.

In some embodiments, the first internal linker has a bridging length of about 15-21 atoms. In some embodiments, the first internal linker substitutes for 4, 5, 6, 7, 8, 9, 10, 11, or 12 nucleotides of the repeat-anti-repeat region of the gRNA. In some embodiments, the first internal linker is in a hairpin between a first portion of the sgRNA and a second portion of the repeat-anti-repeat region, wherein the first portion and the second portion together form a duplex portion.

In some embodiments, the first internal linker substitutes for a loop, or part thereof, of the hairpin of the repeat-anti-repeat region. In some embodiments, the first internal linker substitutes for the loop and the stem, or part thereof, of the hairpin of the repeat-anti-repeat region.

In some embodiments, the first internal linker substitutes for 2, 3, or 4 nucleotides of the loop of the hairpin structure of the repeat-anti-repeat region. In some embodiments, the first internal linker substitutes for the loop of the hairpin structure and at least 2, 4, 6, 8, 10, or 12 nucleotides of the stem of the hairpin structure of the repeat-anti-repeat region. In some embodiments, the first internal linker substitutes for the loop of the hairpin structure and 1, 2, 3, 4, 5, or 6 base pairs of the stem of the hairpin structure of the repeat-anti-repeat region. In some embodiments, the first internal linker substitutes for all of the nucleotides constituting the loop of the hairpin structure of the repeat-anti-repeat region. In some embodiments, the first internal linker substitutes for all of the nucleotides constituting the loop and the stem of the hairpin structure of the upper stem region repeat-anti-repeat region (i.e., the portion of the repeat-anti-repeat region above the bulge). In some embodiments, the second internal linker has a bridging length of about 9-30, optionally about 15-21 atoms. In some embodiments, the second internal linker substitutes for 3, 4, 5, 6, 7, 8, 9, 10, 11, 12, 13, 14, or 15 nucleotides of the hairpin 2 of the gRNA. In some embodiments, the second internal linker substitutes for a loop region of the hairpin 2. In some embodiments, the second internal linker substitutes for a loop region and part of a stem region of the hairpin 2. In some embodiments, the second internal linker substitutes for a loop, or part thereof, of the hairpin 2. In some embodiments, the second internal linker substitutes for the loop and the stem, or part thereof, of the hairpin 2. In some embodiments, the second internal linker substitutes for 2, 3, or 4 nucleotides of the loop of the hairpin 2. In some embodiments, the second internal linker substitutes for all of the nucleotides constituting the loop of the hairpin 2. In some embodiments, the second internal linker substitutes for the loop of the hairpin 2 and at least 1, 2, 3, 4, 5, or 6 nucleotides of the stem of the hairpin 2. In some embodiments, the second internal linker substitutes for the loop of the hairpin and 1, 2, or 3 base pairs of the stem of the hairpin 2.

In some embodiments, the sgRNA comprises a sequence of SEQ ID NO: 204. In some embodiments, nucleotides 41-44 are substituted for the first internal linker relative SEQ ID NO: 204. In some embodiments, nucleotides 101-103 are substituted for the second internal linker relative SEQ ID NO: 204. In some embodiments, 2-18 nucleotides within nucleotides 94-111 are substituted relative to SEQ ID NO: 204.

In some embodiments, the guide RNA is a A. cellulolyticus Cas9 (“AceCas9”) guide RNA. In some embodiments, the guide RNA is a modified AceCas9 guide RNA. In some embodiments, the guide RNA comprises a nucleic acid sequence of SEQ ID NO: 206. In some embodiments, the guide RNA comprises a nucleic acid sequence of SEQ ID NO: 206, including modifications disclosed elsewhere herein.

In some embodiments, the guide RNA is a Campylobacter jejuni Cas9 (“CjeCas9”) guide RNA. In some embodiments, the guide RNA is a modified CjeCas9 guide RNA.

In some embodiments, a gRNA comprises a guide region and a conserved portion 3′ to the guide region, wherein the conserved portion comprises a repeat-anti-repeat region and a hairpin region, and comprises an internal linker substituting for at least 4 nucleotides of the repeat-anti-repeat region. In some embodiments, the first internal linker has a bridging length of about 9-30 atoms, optionally about 15-21 atoms. In some embodiments, the first internal linker substitutes for 4, 5, 6, 7, 8, 9, 10, 11, or 12 nucleotides of the repeat-anti-repeat region of the gRNA. In some embodiments, the first internal linker is in a hairpin structure between a first portion of the sgRNA and a second portion of the repeat-anti-repeat region, wherein the first portion and the second portion together form a duplex portion.

In some embodiments, the guide RNA comprises a nucleic acid sequence of SEQ ID NO: 207. In some embodiments, the guide RNA comprises a nucleic acid sequence of SEQ ID NO: 207, including modifications disclosed elsewhere herein. In some embodiments, wherein nucleotides 33-36 are substituted for the internal linker relative to SEQ ID NO: 207. In some embodiments, 1, 2, 3, 4, 5, or 6 base pairs of nucleotides 27-32 and 37-42 are substituted for the internal linker relative to SEQ ID NO: 207.

In some embodiments, the Cpf1 guide RNA is a Francisella novicida Cas9 (“FnoCas9”) guide RNA. In some embodiments, the guide RNA is a modified FnoCas9guide RNA.

In some embodiments, a gRNA comprises a repeat-anti-repeat region, and an internal linker substituting for at least 4 nucleotides of the repeat-anti-repeat region. In some embodiments, the internal linker has a bridging length of about 9-30 atoms, optionally about 15-21 atoms. In some embodiments, the internal linker substitutes for 3, 4, 5, or 6 nucleotides of the repeat-anti-repeat region of the gRNA.

In some embodiments, the guide RNA comprises a nucleic acid sequence of SEQ ID NO: 208. In some embodiments, the guide RNA comprises a nucleic acid sequence of SEQ ID NO: 208, including modifications disclosed elsewhere herein. In some embodiments, 2, 3, or 4 of nucleotides 40-43 are substituted for the internal linker relative SEQ ID NO: 208. In some embodiments, wherein nucleotides 39-44 are substituted for the internal linker relative SEQ ID NO: 208.

Type VI, Cpf1 Guide RNAs

In some embodiments, the gRNA is a Cpf1 guide RNA. In some embodiments, the guide RNA is a AsCpf1/Cas12a guide RNA. An exemplary AsCpf1/Cas12a sgRNA is shown in FIG. 10C. In some embodiments, the guide RNA is a modified AsCpf1/Cas12a guide RNA. In some embodiments, the guide RNA comprises a nucleic acid sequence of SEQ ID NO: 209. In some embodiments, the guide RNA comprises a nucleic acid sequence of SEQ ID NO: 209, including modifications disclosed elsewhere herein. In some embodiments, the gRNA comprises a sequence of SEQ ID NO: 209 and nucleotides 11-14, 12-15, or optionally 12-14 are substituted for the internal linker relative SEQ ID NO: 209.

In some embodiments, the guide RNA is a Eubacterium siraeum (Es) Cas13d (EsCas13d) guide RNA. An exemplary EsCas13d sgRNA is shown in FIG. 10D. In some embodiments, the guide RNA comprises a nucleic acid sequence of SEQ ID NO: 210. In some embodiments, the guide RNA comprises a nucleic acid sequence of SEQ ID NO: 210 including modifications disclosed elsewhere herein. In some embodiments, the gRNA comprises a sequence of SEQ ID NO: 210 and nucleotides 9-16, or optionally 10-15, or at least 2 nucleotides thereof; are substituted for the internal linker relative to SEQ ID NO: 210.

An exemplary Nme sgRNA is shown in FIG. 10E and various embodiments are provided below.

Various exemplary sgRNAs comprising at least one internal linker are provided in Tables 2A-2B. Nucleotide modifications are indicated in Tables 2A-2B as follows: m: 2′-OMe; *: PS linkage. Thus, for example, mA represents 2′-O-methyl adenosine.

When unmodified nucleotide sequences are provided, A, C, G, and U are independently unmodified or modified RNA nucleotides. When modified nucleotide sequences are provided, in certain embodiments, A, C, G, and U unmodified RNA nucleotides. When modified nucleotide sequences are provided, in certain embodiments, A, C, G, and U are independently unmodified or modified RNA nucleotides.

In the tables herein, L1 and L2, are optionally, C₉ and C₁₈, respectively as follows:

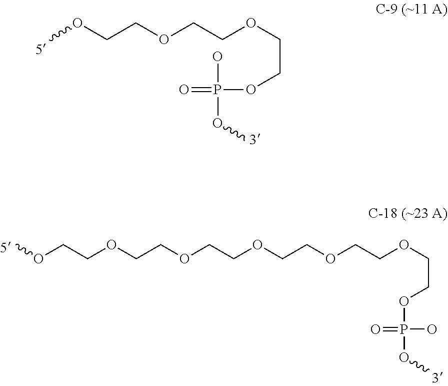

sgRNA designations are sometimes provided with one or more leading zeroes immediately following the G. This does not affect the meaning of the designation. Thus, for example, G000282, G0282, G00282, and G282 refer to the same sgRNA. Similarly, crRNA and or trRNA designations are sometimes provided with one or more leading zeroes immediately following the CR or TR, respectively, which does not affect the meaning of the designation. Thus, for example, CR000100, CR00100, CR0100, and CR100 refer to the same crRNA, and TR000200, TR00200, TR0200, and TR200 refer to the same trRNA.

Exemplary SpyCas9 guide RNAs comprising internal linkers are provided in Tables 2A-2C. As used herein, “Linker 1” or “L1” refers to an internal linker having a bridging length of about 15-21 atoms. As used herein, “Linker 2” or “L2” refers to an internal linker having a bridging length of about 6-12 atoms (e.g., about 9 atoms); “Linker 3” or “L3” refers to an internal linker has a bridging length of about 6 atoms; “Linker 4” or “L4” refers to an internal linker has a bridging length of about 3 atoms; “dS” refers to an abasic nucleoside

TABLE 2A
Table of exemplary gRNA Sequences

| | SEQ | | SEQ | |
|---|---|---|---|---|
| Guide | ID | sgRNA unmodified | ID | |
| ID | NO. | sequence | NO. | sgRNA modified sequence |
| G022497 | 1 | ACGCAAAUAUCAGUCCAGCGGU | 101 | mA*mC*mG*CAAAUAUCAGUCCAGCGG |
| | | UUUAGAGCUA(L1)UAGCAAGU | | UUUUAGAmGmCmUmA(L1)mUmAmGmC |
| | | UAAAAUAAGGC(L2)GUCCGUU | | AAGUUAAAAUAAGGC(L2)GUCCGUUA |
| | | AUCAC(L1)GGGCACCGAGUCG | | UCAC(L1)GGGCACCGAGUCGG*mU*m |
| | | GUGC | | G*mC |
| G022498 | 2 | ACGCAAAUAUCAGUCCAGCGGU | 102 | mA*mC*mG*CAAAUAUCAGUCCAGCGG |
| | | UUUAGAGCUA(L1)UAGCAAGU | | UUUUAGAmGmCmUmA(L1)mUmAmGmC |
| | | UAAAAUAAGGC(L2)GUCCGUU | | AAGUUAAAAUAAGGC(L2)GUCCGUUA |
| | | AUCA(L1)GGCACCGAGUCGGU | | UCA(L1)GGCACCGAGUCGG*mU*mG* |
| | | GC | | mc |
| G022499 | 3 | ACGCAAAUAUCAGUCCAGCGGU | 103 | mA*mC*mG*CAAAUAUCAGUCCAGCGG |
| | | UUUAGAGCU(L1)AGCAAGUUA | | UUUUAGAmGmCmU(L1)mAmGmCAAGU |
| | | AAAUAAGGC(L2)GUCCGUUAU | | UAAAAUAAGGC(L2)GUCCGUUAUCAC |
| | | CAC(L1)GGGCACCGAGUCGGU | | (L1)GGGCACCGAGUCGG*mU*mG*mC |
| | | GC | | |
| G022500 | 4 | ACGCAAAUAUCAGUCCAGCGGU | 104 | mA*mC*mG*CAAAUAUCAGUCCAGCGG |
| | | UUUAGAGCU(L1)AGCAAGUUA | | UUUUAGAmGmCmU(L1)mAmGmCAAGU |
| | | AAAUAAGGC(L2)GUCCGUUAU | | UAAAAUAAGGC(L2)GUCCGUUAUCA |
| | | CA(L1)GGCACCGAGUCGGUGC | | (L1)GGCACCGAGUCGG*mU*mG*mC |
| G022501 | 5 | ACACAAAUACCAGUCCAGCGGU | 105 | mA*mC*mA*CAAAUACCAGUCCAGCGG |
| | | UUUAGAGCUA(L1)UAGCAAGU | | UUUUAGAmGmCmUmA(L1)mUmAmGmC |
| | | UAAAAUAAGGC(L2)GUCCGUU | | AAGUUAAAAUAAGGC(L2)GUCCGUUA |
| | | AUCAC(L1)GGGCACCGAGUCG | | UCAC(L1)GGGCACCGAGUCGG*mU*m |
| | | GUGC | | G*mC |
| G022502 | 6 | ACACAAAUACCAGUCCAGCGGU | 106 | mA*mC*mA*CAAAUACCAGUCCAGCGG |
| | | UUUAGAGCUA(L1)UAGCAAGU | | UUUUAGAmGmCmUmA(L1)mUmAmGmC |
| | | UAAAAUAAGGC(L2)GUCCGUU | | AAGUUAAAAUAAGGC(L2)GUCCGUUA |
| | | AUCA(L1)GGCACCGAGUCGGU | | UCA(L1)GGCACCGAGUCGG*mU*mG* |
| | | GC | | mc |
| G022503 | 7 | ACACAAAUACCAGUCCAGCGGU | 107 | mA*mC*mA*CAAAUACCAGUCCAGCGG |
| | | UUUAGAGCU(L1)AGCAAGUUA | | UUUUAGAmGmCmU(L1)mAmGmCAAGU |
| | | AAAUAAGGC(L2)GUCCGUUAU | | UAAAAUAAGGC(L2)GUCCGUUAUCAC |
| | | CAC(L1)GGGCACCGAGUCGGU | | (L1)GGGCACCGAGUCGG*mU*mG*mC |
| | | GC | | |
| G022504 | 8 | ACACAAAUACCAGUCCAGCGGU | 108 | mA*mC*mA*CAAAUACCAGUCCAGCGG |
| | | UUUAGAGCU(L1)AGCAAGUUA | | UUUUAGAmGmCmU(L1)mAmGmCAAGU |
| | | AAAUAAGGC(L2)GUCCGUUAU | | UAAAAUAAGGC(L2)GUCCGUUAUCA |
| | | CA(L1)GGCACCGAGUCGGUGC | | (L1)GGCACCGAGUCGG*mU*mG*mC |
| G018631 | 9 | ACGCAAAUAUCAGUCCAGCGGU | 109 | mA*mC*mG*CAAAUAUCAGUCCAGCGG |
| (ctrl) | | UUUAGAGCUAGAAAUAGCAAGU | | UUUUAGAmGmCmUmAmGmAmAmAmUmA |
| | | UAAAAUAAGGCUAGUCCGUUAU | | mGmCAAGUUAAAAUAAGGCUAGUCCGU |
| | | CACGAAAGGGCACCGAGUCGGU | | UAUCACGAAAGGGCACCGAGUCGG*mU |
| | | GC | | *mG*mC |
| G017276 | 10 | ACACAAAUACCAGUCCAGCGGU | 110 | mA*mC*mA*CAAAUACCAGUCCAGCGG |
| (ctrl) | | UUUAGAGCUAGAAAUAGCAAGU | | UUUUAGAmGmCmUmAmGmAmAmAmUmA |
| | | UAAAAUAAGGCUAGUCCGUUAU | | mGmCAAGUUAAAAUAAGGCUAGUCCGU |
| | | CACGAAAGGGCACCGAGUCGGU | | UAUCACGAAAGGGCACCGAGUCGG*mU |
| | | GC | | *mG*mC |
| G000502 | 11 | ACACAAAUACCAGUCCAGCGGU | 111 | mA*mC*mA*CAAAUACCAGUCCAGCGG |
| (ctrl) | | UUUAGAGCUAGAAAUAGCAAGU | | UUUUAGAmGmCmUmAmGmAmAmAmUmA |
| | | UAAAAUAAGGCUAGUCCGUUAU | | mGmCAAGUUAAAAUAAGGCUAGUCCGU |
| | | CAACUUGAAAAAGUGGCACCGA | | UAUCAmAmCmUmUmGmAmAmAmAmAmG |
| | | GUCGGUGCUUUU | | mUmGmGmCmAmCmCmGmAmGmUmCmGm |
| | | | | GmUmGmCmU*mU*mU*mU |
| G000534 | 12 | ACGCAAAUAUCAGUCCAGCGGU | 112 | mA*mC*mG*CAAAUAUCAGUCCAGCGG |
| (ctrl) | | UUUAGAGCUAGAAAUAGCAAGU | | UUUUAGAmGmCmUmAmGmAmAmAmUmA |
| | | UAAAAUAAGGCUAGUCCGUUAU | | mGmCAAGUUAAAAUAAGGCUAGUCCGU |
| | | CAACUUGAAAAAGUGGCACCGA | | UAUCAmAmCmUmUmGmAmAmAmAmAmG |
| | | GUCGGUGCUUUU | | mUmGmGmCmAmCmCmGmAmGmUmCmGm |
| | | | | GmUmGmCmU*mU*mU*mU |
| G012401 | 13 | ACACAAAUACCAGUCCAGCGGU | 113 | mA*mC*mA*CAAAUACCAGUCCAGCGG |
| | | UUUAGAGCUAGAAAUAGCAAGU | | UUUUAGAmGmCmUmAmGmAmAmAmUmA |
| | | UAAAAUAAGGCUAGUCCGUUAU | | mGmCAAGUUAAAAUAAGGCUAGUCCGU |
| | | CAACUUGGCACCGAGUCGGUGC | | UAUCAACUUGGCACCGAGUCGG*mU*m |
| | | | | G*mC |
| G017278 | 14 | ACACAAAUACCAGUCCAGCGGU | 114 | mA*mC*mA*CAAAUACCAGUCCAGCGG |
| | | UUUAGAGCUAGAAAUAGCAAGU | | UUUUAGAmGmCmUmAmGmAmAmAmUmA |
| | | UAAAAUAAGGCUAGUCCGUUAU | | mGmCAAGUUAAAAUAAGGCUAGUCCGU |
| | | CACAAGGGCACCGAGUCGGUGC | | UAUCACAAGGGCACCGAGUCGG*mU*m |
| | | | | G*mC |

TABLE 2B
Additional exemplary gRNA sequences

| | SEQ | | SEQ | |
|---|---|---|---|---|
| Guide | ID | sgRNA unmodified | ID | |
| ID | NO. | sequence | NO. | sgRNA modified sequence |
| G018666 | 20 | ACACAAAUACCAGUCCAGCGG | 120 | mA*mC*mA*CAAAUACCAGUCCAGCGGU |
| | | UUUUAGAGCUAG(L2)UAGCA | | UUUAGAmGmCmUmAmG(L2)mUmAmGmC |
| | | AGUUAAAAUAAGGCUAGUCCG | | AAGUUAAAAUAAGGCUAGUCCGUUAUCA |
| | | UUAUCAACUUGGCACCGAGUC | | ACUUGGCACCGAGUCGGmU*mG*mC*mU |
| | | GGUGCU | | |
| G018667 | 21 | ACACAAAUACCAGUCCAGCGG | 121 | mA*mC*mA*CAAAUACCAGUCCAGCGGU |
| | | UUUUAGAGCUA(L2)UAGCAA | | UUUAGAmGmCmUmA(L2)mUmAmGmCAA |
| | | GUUAAAAUAAGGCUAGUCCGU | | GUUAAAAUAAGGCUAGUCCGUUAUCAAC |
| | | UAUCAACUUGGCACCGAGUCG | | UUGGCACCGAGUCGGmU*mG*mC*mU |
| | | GUGCU | | |
| G018668 | 22 | ACACAAAUACCAGUCCAGCGG | 122 | mA*mC*mA*CAAAUACCAGUCCAGCGGU |
| | | UUUUAGAGCUG(L2)AGCAAG | | UUUAGAmGmCmUmG(L2)mAmGmCAAGU |
| | | UUAAAAUAAGGCUAGUCCGUU | | UAAAAUAAGGCUAGUCCGUUAUCAACUU |
| | | AUCAACUUGGCACCGAGUCGG | | GGCACCGAGUCGGmU*mG*mC*mU |
| | | UGCU | | |
| G018669 | 23 | ACACAAAUACCAGUCCAGCGG | 123 | mA*mC*mA*CAAAUACCAGUCCAGCGGU |
| | | UUUUAGAGCU(L2)AGCAAGU | | UUUAGAmGmCmU(L2)mAmGmCAAGUUA |
| | | UAAAAUAAGGCUAGUCCGUUA | | AAAUAAGGCUAGUCCGUUAUCAACUUGG |
| | | UCAACUUGGCACCGAGUCGGU | | CACCGAGUCGGmU*mG*mC*mU |
| | | GCU | | |
| G018670 | 24 | ACACAAAUACCAGUCCAGCGG | 124 | mA*mC*mA*CAAAUACCAGUCCAGCGGU |
| | | UUUUAGAGC(L2)GCAAGUUA | | UUUAGAmGmC(L2)mGmCAAGUUAAAAU |
| | | AAAUAAGGCUAGUCCGUUAUC | | AAGGCUAGUCCGUUAUCAACUUGGCACC |
| | | AACUUGGCACCGAGUCGGUGC | | GAGUCGGmU*mG*mC*mU |
| | | U | | |
| G018671 | 25 | ACACAAAUACCAGUCCAGCGG | 125 | mA*mC*mA*CAAAUACCAGUCCAGCGGU |
| | | UUUUAGAGCG(L2)GCAAGUU | | UUUAGAmGmCmG(L2)mGmCAAGUUAAA |
| | | AAAAUAAGGCUAGUCCGUUAU | | AUAAGGCUAGUCCGUUAUCAACUUGGCA |
| | | CAACUUGGCACCGAGUCGGUG | | CCGAGUCGGmU*mG*mC*mU |
| | | CU | | |
| G018672 | 26 | ACACAAAUACCAGUCCAGCGG | 126 | mA*mC*mA*CAAAUACCAGUCCAGCGGU |
| | | UUUUAGAG(L2)CAAGUUAAA | | UUUAGAmG(L2)mCAAGUUAAAAUAAGG |
| | | AUAAGGCUAGUCCGUUAUCAA | | CUAGUCCGUUAUCAACUUGGCACCGAGU |
| | | CUUGGCACCGAGUCGGUGCU | | CGGmU*mG*mC*mU |
| G018673 | 27 | ACACAAAUACCAGUCCAGCGG | 127 | mA*mC*mA*CAAAUACCAGUCCAGCGGU |
| | | UUUUAGAGCUAG(L1)UAGCA | | UUUAGAmGmCmUmAmG(L1)mUmAmGmC |
| | | AGUUAAAAUAAGGCUAGUCCG | | AAGUUAAAAUAAGGCUAGUCCGUUAUCA |
| | | UUAUCAACUUGGCACCGAGUC | | ACUUGGCACCGAGUCGGmU*mG*mC*mU |
| | | GGUGCU | | |
| G018674 | 28 | ACACAAAUACCAGUCCAGCGG | 128 | mA*mC*mA*CAAAUACCAGUCCAGCGGU |
| | | UUUUAGAGCUA(L1)UAGCAA | | UUUAGAmGmCmUmA(L1)mUmAmGmCAA |
| | | GUUAAAAUAAGGCUAGUCCGU | | GUUAAAAUAAGGCUAGUCCGUUAUCAAC |
| | | UAUCAACUUGGCACCGAGUCG | | UUGGCACCGAGUCGGmU*mG*mC*mU |
| | | GUGCU | | |
| G018675 | 29 | ACACAAAUACCAGUCCAGCGG | 129 | mA*mC*mA*CAAAUACCAGUCCAGCGGU |
| | | UUUUAGAGCUG(L1)AGCAAG | | UUUAGAmGmCmUmG(L1)mAmGmCAAGU |
| | | UUAAAAUAAGGCUAGUCCGUU | | UAAAAUAAGGCUAGUCCGUUAUCAACUU |
| | | AUCAACUUGGCACCGAGUCGG | | GGCACCGAGUCGGmU*mG*mC*mU |
| | | UGCU | | |
| G018676 | 30 | ACACAAAUACCAGUCCAGCGG | 130 | mA*mC*mA*CAAAUACCAGUCCAGCGGU |
| | | UUUUAGAGCU(L1)AGCAAGU | | UUUAGAmGmCmU(L1)mAmGmCAAGUUA |
| | | UAAAAUAAGGCUAGUCCGUUA | | AAAUAAGGCUAGUCCGUUAUCAACUUGG |
| | | UCAACUUGGCACCGAGUCGGU | | CACCGAGUCGGmU*mG*mC*mU |
| | | GCU | | |
| G018677 | 31 | ACACAAAUACCAGUCCAGCGG | 131 | mA*mC*mA*CAAAUACCAGUCCAGCGGU |
| | | UUUUAGAGCG(L1)GCAAGUU | | UUUAGAmGmCmG(L1)mGmCAAGUUAAA |
| | | AAAAUAAGGCUAGUCCGUUAU | | AUAAGGCUAGUCCGUUAUCAACUUGGCA |
| | | CAACUUGGCACCGAGUCGGUG | | CCGAGUCGGmU*mG*mC*mU |
| | | CU | | |
| G018678 | 32 | ACACAAAUACCAGUCCAGCGG | 132 | mA*mC*mA*CAAAUACCAGUCCAGCGGU |
| | | UUUUAGAGC(L1)GCAAGUUA | | UUUAGAmGmC(L1)mGmCAAGUUAAAAU |
| | | AAAUAAGGCUAGUCCGUUAUC | | AAGGCUAGUCCGUUAUCAACUUGGCACC |
| | | AACUUGGCACCGAGUCGGUGC | | GAGUCGGmU*mG*mC*mU |
| | | U | | |
| G018679 | 33 | ACACAAAUACCAGUCCAGCGG | 133 | mA*mC*mA*CAAAUACCAGUCCAGCGGU |
| | | UUUUAGAG(L1)CAAGUUAAA | | UUUAGAmG(L1)mCAAGUUAAAAUAAGG |
| | | AUAAGGCUAGUCCGUUAUCAA | | CUAGUCCGUUAUCAACUUGGCACCGAGU |
| | | CUUGGCACCGAGUCGGUGCU | | CGGmU*mG*mC*mU |
| G018680 | 34 | ACACAAAUACCAGUCCAGCGG | 134 | mA*mC*mA*CAAAUACCAGUCCAGCGGU |
| | | UUUUAGAGCUAGAAAUAGCAA | | UUUAGAmGmCmUmAmGmAmAmAmUmAmG |
| | | GUUAAAAUAAGGC(L4)GUCC | | mCAAGUUAAAAUAAGGC(L4)GUCCGUU |
| | | GUUAUCAACUUGGCACCGAGU | | AUCAACUUGGCACCGAGUCGGmU*mG*m |
| | | CGGUGCU | | C*mU |
| G018681 | 35 | ACACAAAUACCAGUCCAGCGG | 135 | mA*mC*mA*CAAAUACCAGUCCAGCGGU |
| | | UUUUAGAGCUAGAAAUAGCAA | | UUUAGAmGmCmUmAmGmAmAmAmUmAmG |
| | | GUUAAAAUAAGGC(L2)GUCC | | mCAAGUUAAAAUAAGGC(L2)GUCCGUU |
| | | GUUAUCAACUUGGCACCGAGU | | AUCAACUUGGCACCGAGUCGGmU*mG*m |
| | | CGGUGCU | | C*mU |
| G018682 | 36 | ACACAAAUACCAGUCCAGCGG | 136 | mA*mC*mA*CAAAUACCAGUCCAGCGGU |
| | | UUUUAGAGCUAGAAAUAGCAA | | UUUAGAmGmCmUmAmGmAmAmAmUmAmG |
| | | GUUAAAAUAAGGC(L1)GUCC | | mCAAGUUAAAAUAAGGC(L1)GUCCGUU |
| | | GUUAUCAACUUGGCACCGAGU | | AUCAACUUGGCACCGAGUCGGmU*mG*m |
| | | CGGUGCU | | C*mU |
| G018683 | 37 | ACACAAAUACCAGUCCAGCGG | 137 | mA*mC*mA*CAAAUACCAGUCCAGCGGU |
| | | UUUUAGAGCUAGAAAUAGCAA | | UUUAGAmGmCmUmAmGmAmAmAmUmAmG |
| | | GUUAAAAUAAGGCUAGUCCGU | | mCAAGUUAAAAUAAGGCUAGUCCGUUAU |
| | | UAUCAAC(L2)UGGCACCGAG | | CAAC(L2)UGGCACCGAGUCGGmU*mG* |
| | | UCGGUGCU | | mC*mU |
| G018684 | 38 | ACACAAAUACCAGUCCAGCGG | 138 | mA*mC*mA*CAAAUACCAGUCCAGCGGU |
| | | UUUUAGAGCUAGAAAUAGCAA | | UUUAGAmGmCmUmAmGmAmAmAmUmAmG |
| | | GUUAAAAUAAGGCUAGUCCGU | | mCAAGUUAAAAUAAGGCUAGUCCGUUAU |
| | | UAUCAAC(L2)GGCACCGAGU | | CAAC(L2)GGCACCGAGUCGGmU*mG*m |
| | | CGGUGCU | | C*mU |
| G018685 | 39 | ACACAAAUACCAGUCCAGCGG | 139 | mA*mC*mA*CAAAUACCAGUCCAGCGGU |
| | | UUUUAGAGCUAGAAAUAGCAA | | UUUAGAmGmCmUmAmGmAmAmAmUmAmG |
| | | GUUAAAAUAAGGCUAGUCCGU | | mCAAGUUAAAAUAAGGCUAGUCCGUUAU |
| | | UAUCAA(L2)UGGCACCGAGU | | CAA(L2)UGGCACCGAGUCGGmU*mG*m |
| | | CGGUGCU | | C*mU |
| G018686 | 40 | ACACAAAUACCAGUCCAGCGG | 140 | mA*mC*mA*CAAAUACCAGUCCAGCGGU |
| | | UUUUAGAGCUAGAAAUAGCAA | | UUUAGAmGmCmUmAmGmAmAmAmUmAmG |
| | | GUUAAAAUAAGGCUAGUCCGU | | mCAAGUUAAAAUAAGGCUAGUCCGUUAU |
| | | UAUCAA(L2)GGCACCGAGUC | | CAA(L2)GGCACCGAGUCGGmU*mG*mC |
| | | GGUGCU | | *mU |
| G018687 | 41 | ACACAAAUACCAGUCCAGCGG | 141 | mA*mC*mA*CAAAUACCAGUCCAGCGGU |
| | | UUUUAGAGCUAGAAAUAGCAA | | UUUAGAmGmCmUmAmGmAmAmAmUmAmG |
| | | GUUAAAAUAAGGCUAGUCCGU | | mCAAGUUAAAAUAAGGCUAGUCCGUUAU |
| | | UAUCAAC(L1)UGGCACCGAG | | CAAC(L1)UGGCACCGAGUCGGmU*mG* |
| | | UCGGUGCU | | mC*mU |
| G018688 | 42 | ACACAAAUACCAGUCCAGCGG | 142 | mA*mC*mA*CAAAUACCAGUCCAGCGGU |
| | | UUUUAGAGCUAGAAAUAGCAA | | UUUAGAmGmCmUmAmGmAmAmAmUmAmG |
| | | GUUAAAAUAAGGCUAGUCCGU | | mCAAGUUAAAAUAAGGCUAGUCCGUUAU |
| | | UAUCAAC(L1)GGCACCGAGU | | CAAC(L1)GGCACCGAGUCGGmU*mG*m |
| | | CGGUGCU | | C*mU |
| G018689 | 43 | ACACAAAUACCAGUCCAGCGG | 143 | mA*mC*mA*CAAAUACCAGUCCAGCGGU |
| | | UUUUAGAGCUAGAAAUAGCAA | | UUUAGAmGmCmUmAmGmAmAmAmUmAmG |
| | | GUUAAAAUAAGGCUAGUCCGU | | mCAAGUUAAAAUAAGGCUAGUCCGUUAU |
| | | UAUCAA(L1)UGGCACCGAGU | | CAA(L1)UGGCACCGAGUCGGmU*mG*m |
| | | CGGUGCU | | C*mU |
| G018690 | 44 | ACACAAAUACCAGUCCAGCGG | 144 | mA*mC*mA*CAAAUACCAGUCCAGCGGU |
| | | UUUUAGAGCUAGAAAUAGCAA | | UUUAGAmGmCmUmAmGmAmAmAmUmAmG |
| | | GUUAAAAUAAGGCUAGUCCGU | | mCAAGUUAAAAUAAGGCUAGUCCGUUAU |
| | | UAUCAA(L1)GGCACCGAGUC | | CAA(L1)GGCACCGAGUCGGmU*mG*mC |
| | | GGUGCU | | *mU |
| G018691 | 45 | ACACAAAUACCAGUCCAGCGG | 145 | mA*mC*mA*CAAAUACCAGUCCAGCGGU |
| | | UUUUAGAGCUA(L1)UAGCAA | | UUUAGAmGmCmUmA(L1)mUmAmGmCAA |
| | | GUUAAAAUAAGGC(L2)GUCC | | GUUAAAAUAAGGC(L2)GUCCGUUAUCA |
| | | GUUAUCAACUUGGCACCGAGU | | ACUUGGCACCGAGUCGGmU*mG*mC*mU |
| | | CGGUGCU | | |
| G018692 | 46 | ACACAAAUACCAGUCCAGCGG | 146 | mA*mC*mA*CAAAUACCAGUCCAGCGGU |
| | | UUUUAGAGCUA(L1)UAGCAA | | UUUAGAmGmCmUmA(L1)mUmAmGmCAA |
| | | GUUAAAAUAAGGC(L1)GUCC | | GUUAAAAUAAGGC(L1)GUCCGUUAUCA |
| | | GUUAUCAACUUGGCACCGAGU | | ACUUGGCACCGAGUCGGmU*mG*mC*mU |
| | | CGGUGCU | | |
| G018693 | 47 | ACACAAAUACCAGUCCAGCGG | 147 | mA*mC*mA*CAAAUACCAGUCCAGCGGU |
| | | UUUUAGAGCUA(L1)UAGCAA | | UUUAGAmGmCmUmA(L1)mUmAmGmCAA |
| | | GUUAAAAUAAGGCUAGUCCGU | | GUUAAAAUAAGGCUAGUCCGUUAUCAA |
| | | UAUCAA(L2)UGGCACCGAGU | | (L2)UGGCACCGAGUCGGmU*mG*mC*m |
| | | CGGUGCU | | U |
| G018694 | 48 | ACACAAAUACCAGUCCAGCGG | 148 | mA*mC*mA*CAAAUACCAGUCCAGCGGU |
| | | UUUUAGAGCUA(L1)UAGCAA | | UUUAGAmGmCmUmA(L1)mUmAmGmCAA |
| | | GUUAAAAUAAGGCUAGUCCGU | | GUUAAAAUAAGGCUAGUCCGUUAUCAA |
| | | UAUCAA(L1)UGGCACCGAGU | | (L1)UGGCACCGAGUCGGmU*mG*mC*m |
| | | CGGUGCU | | U |
| G018695 | 49 | ACACAAAUACCAGUCCAGCGG | 149 | mA*mC*mA*CAAAUACCAGUCCAGCGGU |
| | | UUUUAGAGCUA(L1)UAGCAA | | UUUAGAmGmCmUmA(L1)mUmAmGmCAA |
| | | GUUAAAAUAAGGC(L2)GUCC | | GUUAAAAUAAGGC(L2)GUCCGUUAUCA |
| | | GUUAUCAA(L2)UGGCACCGA | | A(L2)UGGCACCGAGUCGGmU*mG*mC* |
| | | GUCGGUGCU | | mU |
| G018696 | 50 | ACACAAAUACCAGUCCAGCGG | 150 | mA*mC*mA*CAAAUACCAGUCCAGCGGU |
| | | UUUUAGAGCUA(L1)UAGCAA | | UUUAGAmGmCmUmA(L1)mUmAmGmCAA |
| | | GUUAAAAUAAGGC(L1)GUCC | | GUUAAAAUAAGGC(L1)GUCCGUUAUCA |
| | | GUUAUCAA(L1)UGGCACCGA | | A(L1)UGGCACCGAGUCGGmU*mG*mC* |
| | | GUCGGUGCU | | mU |
| G018697 | 51 | ACACAAAUACCAGUCCAGCGG | 151 | mA*mC*mA*CAAAUACCAGUCCAGCGGU |
| | | UUUUAGAGCUAG(L1)UAGCA | | UUUAGAmGmCmUmAmG(L1)mUmAmGmC |
| | | AGUUAAAAUAAGGC(L2)GUC | | AAGUUAAAAUAAGGC(L2)GUCCGUUAU |
| | | CGUUAUCAA(L2)UGGCACCG | | CAA(L2)UGGCACCGAGUCGGmU*mG*m |
| | | AGUCGGUGCU | | C*mU |
| G018698 | 52 | ACACAAAUACCAGUCCAGCGG | 152 | mA*mC*mA*CAAAUACCAGUCCAGCGGU |
| | | UUUUAGAGCUAG(L1)UAGCA | | UUUAGAmGmCmUmAmG(L1)mUmAmGmC |
| | | AGUUAAAAUAAGGC(L1)GUC | | AAGUUAAAAUAAGGC(L1)GUCCGUUAU |
| | | CGUUAUCAA(L1)UGGCACCG | | CAA(L1)UGGCACCGAGUCGGmU*mG*m |
| | | AGUCGGUGCU | | C*mU |
| G018699 | 53 | ACACAAAUACCAGUCCAGCGG | 153 | mA*mC*mA*CAAAUACCAGUCCAGCGGU |
| | | UUUUAGAGCU(L1)AGCAAGU | | UUUAGAmGmCmU(L1)mAmGmCAAGUUA |
| | | UAAAAUAAGGC(L2)GUCCGU | | AAAUAAGGC(L2)GUCCGUUAUCAA |
| | | UAUCAA(L2)UGGCACCGAGU | | (L2)UGGCACCGAGUCGGmU*mG*mC*m |
| | | CGGUGCU | | U |
| G018700 | 54 | ACACAAAUACCAGUCCAGCGG | 154 | mA*mC*mA*CAAAUACCAGUCCAGCGGU |
| | | UUUUAGAGCU(L1)AGCAAGU | | UUUAGAmGmCmU(L1)mAmGmCAAGUUA |
| | | UAAAAUAAGGC(L1)GUCCGU | | AAAUAAGGC(L1)GUCCGUUAUCAA |
| | | UAUCAA(L1)UGGCACCGAGU | | (L1)UGGCACCGAGUCGGmU*mG*mC*m |
| | | CGGUGCU | | U |
| G018701 | 55 | ACACAAAUACCAGUCCAGCGG | 155 | mA*mC*mA*CAAAUACCAGUCCAGCGGU |
| | | UUUUAGAGCUG(L1)AGCAAG | | UUUAGAmGmCmUmG(L1)mAmGmCAAGU |
| | | UUAAAAUAAGGC(L2)GUCCG | | UAAAAUAAGGC(L2)GUCCGUUAUCAA |
| | | UUAUCAA(L2)UGGCACCGAG | | (L2)UGGCACCGAGUCGGmU*mG*mC* |
| | | UCGGUGCU | | mU |
| G018702 | 56 | ACACAAAUACCAGUCCAGCGG | 156 | mA*mC*mA*CAAAUACCAGUCCAGCGGU |
| | | UUUUAGAGCUG(L1)AGCAAG | | UUUAGAmGmCmUmG(L1)mAmGmCAAGU |
| | | UUAAAAUAAGGC(L1)GUCCG | | UAAAAUAAGGC(L1)GUCCGUUAUCAA |
| | | UUAUCAA(L1)UGGCACCGAG | | (L1)UGGCACCGAGUCGGmU*mG*mC*m |
| | | UCGGUGCU | | U |
| G018703 | 57 | ACACAAAUACCAGUCCAGCGG | 157 | mA*mC*mA*CAAAUACCAGUCCAGCGGU |
| | | UUUUAGAGC(L1)GCAAGUUA | | UUUAGAmGmC(L1)mGmCAAGUUAAAAU |
| | | AAAUAAGGC(L2)GUCCGUUA | | AAGGC(L2)GUCCGUUAUCAA(L2)UGG |
| | | UCAA(L2)UGGCACCGAGUCG | | CACCGAGUCGGmU*mG*mC*mU |
| | | GUGCU | | |
| G018704 | 58 | ACACAAAUACCAGUCCAGCGG | 158 | mA*mC*mA*CAAAUACCAGUCCAGCGGU |
| | | UUUUAGAGC(L1)GCAAGUUA | | UUUAGAmGmC(L1)mGmCAAGUUAAAAU |
| | | AAAUAAGGC(L1)GUCCGUUA | | AAGGC(L1)GUCCGUUAUCAA(L1)UGG |
| | | UCAA(L1)UGGCACCGAGUCG | | CACCGAGUCGGmU*mG*mC*mU |
| | | GUGCU | | |
| G018705 | 59 | ACACAAAUACCAGUCCAGCGG | 159 | mA*mC*mA*CAAAUACCAGUCCAGCGGU |
| | | UUUUAGAGCUAGAAAUAGCAA | | UUUAGAmGmCmUmAmGmAmAmAmUmAmG |
| | | GUUAAAAUAAGGCUAGUCCGU | | mCAAGUUAAAAUAAGGCUAGUCCGUUAU |
| | | UAUCAC(L2)GGGCACCGAGU | | CAC(L2)GGGCACCGAGUCGGmU*mG*m |
| | | CGGUGCU | | C*mU |
| G018706 | 60 | ACACAAAUACCAGUCCAGCGG | 160 | mA*mC*mA*CAAAUACCAGUCCAGCGGU |
| | | UUUUAGAGCUAGAAAUAGCAA | | UUUAGAmGmCmUmAmGmAmAmAmUmAmG |
| | | GUUAAAAUAAGGCUAGUCCGU | | mCAAGUUAAAAUAAGGCUAGUCCGUUAU |
| | | UAUCAC(L1)GGGCACCGAGU | | CAC(L1)GGGCACCGAGUCGGmU*mG*m |
| | | CGGUGCU | | C*mU |
| G018707 | 61 | ACACAAAUACCAGUCCAGCGG | 161 | mA*mC*mA*CAAAUACCAGUCCAGCGGU |
| | | UUUUAGAGCUAGAAAUAGCAA | | UUUAGAmGmCmUmAmGmAmAmAmUmAmG |
| | | GUUAAAAUAAGGCUAGUCCGU | | mCAAGUUAAAAUAAGGCUAGUCCGUUAU |
| | | UAUCA(L1)GGCACCGAGUCG | | CA(L1)GGCACCGAGUCGGmU*mG*mC* |
| | | GUGCU | | mU |
| G018708 | 62 | ACACAAAUACCAGUCCAGCGG | 162 | mA*mC*mA*CAAAUACCAGUCCAGCGGU |
| | | UUUUAGAGCUAGAAAUAGCAA | | UUUAGAmGmCmUmAmGmAmAmAmUmAmG |
| | | GUUAAAAUAAGGCUAGUCCGU | | mCAAGUUAAAAUAAGGCUAGUCCGUUAU |
| | | UAUCA(L2)GGCACCGAGUCG | | CA(L2)GGCACCGAGUCGGmU*mG*mC* |
| | | GUGCU | | mU |
| G018804 | 63 | ACACAAAUACCAGUCCAGCGG | 163 | mA*mC*mA*CAAAUACCAGUCCAGCGGU |
| | | UUUUAGAGCUAGAAAUAGCAA | | UUUAGAmGmCmUmAmGmAmAmAmUmAmG |
| | | GUUAAAAUAAGGCUAGUCCGU | | mCAAGUUAAAAUAAGGCUAGUCCGUUAU |
| | | UAUCACGAAAGGGCACCGAGU | | CAmCmGmAmAmAmGmGmGmCmAmCmCmG |
| | | CGGUGC | | mAmGmUmCmGmG*mU*mG*mC |
| G018805 | 64 | ACACAAAUACCAGUCCAGCGG | 164 | mA*mC*mA*CAAAUACCAGUCCAGCGGU |
| | | UUUUAGAGCUAGAAAUAGCAA | | UUUAGAmGmCmUmAmGmAmAmAmUmAmG |
| | | GUUAAAAUAAGGCUAGUCCGU | | mCAAGUUAAAAUAAGGCUAGUCCGUUAU |
| | | UAUCACGAAAGGGCACCGAGU | | CACmGmAmAmAmGmGmGmCmAmCmCmGm |
| | | CGGUGC | | AmGmUmCmGmG*mU*mG*mC |
| G018806 | 65 | ACACAAAUACCAGUCCAGCGG | 165 | mA*mC*mA*CAAAUACCAGUCCAGCGGU |
| | | UUUUAGAGCUA(L1)UAGCAA | | UUUAGAmGmCmUmA(L1)mUmAmGmCAA |
| | | GUUAAAAUAAGGCUAGUCCGU | | GUUAAAAUAAGGCUAGUCCGUUAUCACG |
| | | UAUCACGAAAGGGCACCGAGU | | AAAGGGCACCGAGUCGG*mU*mG*mC |
| | | CGGUGC | | |
| G018807 | 66 | ACACAAAUACCAGUCCAGCGG | 166 | mA*mC*mA*CAAAUACCAGUCCAGCGGU |
| | | UUUUAGAGCUAGAAAUAGCAA | | UUUAGAmGmCmUmAmGmAmAmAmUmAmG |
| | | GUUAAAAUAAGGCUAGUCCGU | | mCAAGUUAAAAUAAGGCUAGUCCGUUAU |
| | | UAUCAC(L1)GGGCACCGAGU | | CAC(L1)GGGCACCGAGUCGG*mU*mG* |
| | | CGGUGC | | mc |
| G018808 | 67 | ACACAAAUACCAGUCCAGCGG | 167 | mA*mC*mA*CAAAUACCAGUCCAGCGGU |
| | | UUUUAGAGCUA(L1)UAGCAA | | UUUAGAmGmCmUmA(L1)mUmAmGmCAA |
| | | GUUAAAAUAAGGC(L1)GUCC | | GUUAAAAUAAGGC(L1)GUCCGUUAUCA |
| | | GUUAUCAC(L1)GGGCACCGA | | C(L1)GGGCACCGAGUCGG*mU*mG*mC |
| | | GUCGGUGC | | |
| G018809 | 68 | ACACAAAUACCAGUCCAGCGG | 168 | mA*mC*mA*CAAAUACCAGUCCAGCGGU |
| | | UUUUAGAGCUA(L1)UAGCAA | | UUUAGAmGmCmUmA(L1)mUmAmGmCAA |
| | | GUUAAAAUAAGGCUAGUCCGU | | GUUAAAAUAAGGCUAGUCCGUUAUCAC |
| | | UAUCAC(L1)GGGCACCGAGU | | (L1)GGGCACCGAGUCGG*mU*mG*mC |
| | | CGGUGC | | |
| G018810 | 69 | ACACAAAUACCAGUCCAGCGG | 169 | mA*mC*mA*CAAAUACCAGUCCAGCGGU |
| | | UUUUAGAGCUA(L2)UAGCAA | | UUUAGAmGmCmUmA(L2)mUmAmGmCAA |
| | | GUUAAAAUAAGGCUAGUCCGU | | GUUAAAAUAAGGCUAGUCCGUUAUCACG |
| | | UAUCACGAAAGGGCACCGAGU | | AAAGGGCACCGAGUCGG*mU*mG*mC |
| | | CGGUGC | | |
| G018811 | 70 | ACACAAAUACCAGUCCAGCGG | 170 | mA*mC*mA*CAAAUACCAGUCCAGCGGU |
| | | UUUUAGAGCUAGAAAUAGCAA | | UUUAGAmGmCmUmAmGmAmAmAmUmAmG |
| | | GUUAAAAUAAGGCUAGUCCGU | | mCAAGUUAAAAUAAGGCUAGUCCGUUAU |
| | | UAUCAAAAUGGCACCGAGUCG | | CAmAmAmAmUmGmGmCmAmCmCmGmAmG |
| | | GUGC | | mUmCmGmG*mU*mG*mC |
| G018812 | 71 | ACACAAAUACCAGUCCAGCGG | 171 | mA*mC*mA*CAAAUACCAGUCCAGCGGU |
| | | UUUUAGAGCUAGAAAUAGCAA | | UUUAGAmGmCmUmAmGmAmAmAmUmAmG |
| | | GUUAAAAUAAGGCUAGUCCGU | | mCAAGUUAAAAUAAGGCUAGUCCGUUAU |
| | | UAUCAAAAUGGCACCGAGUCG | | CAAmAmAmUmGmGmCmAmCmCmGmAmGm |
| | | GUGC | | UmCmGmG*mU*mG*mC |
| G018813 | 72 | ACACAAAUACCAGUCCAGCGG | 172 | mA*mC*mA*CAAAUACCAGUCCAGCGGU |
| | | UUUUAGAGCUA(L1)UAGCAA | | UUUAGAmGmCmUmA(L1)mUmAmGmCAA |
| | | GUUAAAAUAAGGCUAGUCCGU | | GUUAAAAUAAGGCUAGUCCGUUAUCAAA |
| | | UAUCAAAAUGGCACCGAGUCG | | AUGGCACCGAGUCGG*mU*mG*mC |
| | | GUGC | | |
| G018814 | 73 | ACACAAAUACCAGUCCAGCGG | 173 | mA*mC*mA*CAAAUACCAGUCCAGCGGU |
| | | UUUUAGAGCUAGAAAUAGCAA | | UUUAGAmGmCmUmAmGmAmAmAmUmAmG |
| | | GUUAAAAUAAGGCUAGUCCGU | | mCAAGUUAAAAUAAGGCUAGUCCGUUAU |
| | | UAUCAA(L1)UGGCACCGAGU | | CAA(L1)UGGCACCGAGUCGG*mU*mG* |
| | | CGGUGC | | mC |
| G018815 | 74 | ACACAAAUACCAGUCCAGCGG | 174 | mA*mC*mA*CAAAUACCAGUCCAGCGGU |
| | | UUUUAGAGCUA(L1)UAGCAA | | UUUAGAmGmCmUmA(L1)mUmAmGmCAA |
| | | GUUAAAAUAAGGCUAGUCCGU | | GUUAAAAUAAGGCUAGUCCGUUAUCAA |
| | | UAUCAA(L1)UGGCACCGAGU | | (L1)UGGCACCGAGUCGG*mU*mG*mC |
| | | CGGUGC | | |
| G018816 | 75 | ACACAAAUACCAGUCCAGCGG | 175 | mA*mC*mA*CAAAUACCAGUCCAGCGGU |
| | | UUUUAGAGC(L1)GCAAGUUA | | UUUAGAmGmC(L1)mGmCAAGUUAAAAU |
| | | AAAUAAGGC(L1)GUCCGUUA | | AAGGC(L1)GUCCGUUAUCAA(L1)UGG |
| | | UCAA(L1)UGGCACCGAGUCG | | CACCGAGUCGG*mU*mG*mC |
| | | GUGC | | |
| G030924 | 77 | GGCCCAGACUGAGCACGUGAG | 177 | mG*mG*mC*CCAGACUGAGCACGUGAGU |
| (target | | UUUUAGA(ds)AAGUUAAAAU | | UUUAGA(ds)AAGUUAAAAUAAGGCUAG |
| gene: | | AAGGCUAGUCCGUUAUCAC | | UCCGUUAUCAC(L1)GGGCACCGAGUCG |
| HEK3) | | (L1)GGGCACCGAGUCGGUGC | | GmU*mG*mC*mU |
| | | U | | |
| G030925 | 78 | GGCCCAGACUGAGCACGUGAG | 178 | mG*mG*mC*CCAGACUGAGCACGUGAGU |
| (target | | UUUUAGA(L4)AAGUUAAAAU | | UUUAGA(S3)AAGUUAAAAUAAGGCUAG |
| gene: | | AAGGCUAGUCCGUUAUCAC | | UCCGUUAUCAC(L1)GGGCACCGAGUCG |
| HEK3) | | (L1)GGGCACCGAGUCGGUGC | | GmU*mG*mC*mU |
| | | U | | |
| G030926 | 79 | GGCCCAGACUGAGCACGUGAG | 179 | mG*mG*mC*CCAGACUGAGCACGUGAGU |
| (target | | UUUUAGA(L3)AAGUUAAAAU | | UUUAGA(L3)AAGUUAAAAUAAGGCUAG |
| gene: | | AAGGCUAGUCCGUUAUCAC | | UCCGUUAUCAC(L1)GGGCACCGAGUCG |
| HEK3) | | (L1)GGGCACCGAGUCGGUGC | | GmU*mG*mC*mU |
| | | U | | |
| G030927 | 80 | GGCCCAGACUGAGCACGUGAG | 180 | mG*mG*mC*CCAGACUGAGCACGUGAGU |
| (target | | UUUUAGA(L2)AAGUUAAAAU | | UUUAGA(L2)AAGUUAAAAUAAGGCUAG |
| gene: | | AAGGCUAGUCCGUUAUCAC | | UCCGUUAUCAC(L1)GGGCACCGAGUCG |
| HEK3) | | (L1)GGGCACCGAGUCGGUGC | | GmU*mG*mC*mU |
| | | U | | |
| G030928 | 81 | GGCCCAGACUGAGCACGUGAG | 181 | mG*mG*mC*CCAGACUGAGCACGUGAGU |
| (target | | UUUUAGA(L1)AAGUUAAAAU | | UUUAGA(L1)AAGUUAAAAUAAGGCUAG |
| gene: | | AAGGCUAGUCCGUUAUCAC | | UCCGUUAUCAC(L1)GGGCACCGAGUCG |
| HEK3) | | (L1)GGGCACCGAGUCGGUGC | | GmU*mG*mC*mU |
| | | U | | |
| G030929 | 82 | GGCCCAGACUGAGCACGUGAG | 182 | mG*mG*mC*CCAGACUGAGCACGUGAGU |
| (target | | UUUUAGAGC(L1)GCAAGUUA | | UUUAGAmGmC(L1)mGmCAAGUUAAAAU |
| gene: | | AAAUAAGGCUAGUCCGUUAUC | | AAGGCUAGUCCGUUAUCAC(L1)GGGCA |
| HEK3) | | AC(L1)GGGCACCGAGUCGGU | | CCGAGUCGGmU*mG*mC*mU |
| | | GCU | | |
| G025989 | 83 | GGCCCAGACUGAGCACGUGAG | 183 | mG*mG*mC*CCAGACUGAGCACGUGAGU |
| (target | | UUUUAGAGCUA(L1)UAGCAA | | UUUAGAmGmCmUmA(L1)mUmAmGmCAA |
| gene: | | GUUAAAAUAAGGCUAGUCCGU | | GUUAAAAUAAGGCUAGUCCGUUAUCAC |
| HEK3) | | UAUCAC(L1)GGGCACCGAGU | | (L1)GGGCACCGAGUCGGmU*mG*mC*m |
| | | CGGUGCU | | U |
| G030930 | 84 | GGCCCAGACUGAGCACGUGAG | 184 | mG*mG*mC*CCAGACUGAGCACGUGAGU |
| (target | | UUUUAGAGCUA(L1)UAGCAA | | UUUAGAmGmCmUmA(L1)mUmAmGmCAA |
| gene: | | GUUAAAAUAAGGCUAGUCCGU | | GUUAAAAUAAGGCUAGUCCGUUAUCAC |
| HEK3) | | UAUCAC(L1)GGGCACCGAGU | | (L1)GGGCACCGAGUmCmGmGmU*mG*m |
| | | CGGUGCU | | C*mU |

TABLE 2C
Exemplary SpyCas9 guide RNAs comprising linkers

| SEQ | |
|---|---|
| ID | |
| NO: | gRNA sequence |
| 211 | NNNNNNNNNNNNNNNNNNNNGUUUUAGAGCUA(L1)UAGCAAGUUA |
| | AAAUAAGGC(L2)GUCCGUUAUCAACUU(L1)AAGUGGCACCGAGU |
| | CGGUGCUUUU |
| 212 | NNNNNNNNNNNNNNNNNNNNGUUUUAGAGCUA(L1)UAGCAAGUUA |
| | AAAUAAGGC(L2)GUCCGUUAUCAC(L1)GGGCACCGAGUCGGUGC |
| 213 | mN*mN*mN*NNNNNNNNNNNNNNNNNGUUUUAGAmGmCmUmA(L1) |
| | mUmAmGmCAAGUUAAAAUAAGGC(L2)GUCCGUUAUCAC(L1)GGG |
| | CACCGAGUCGG*mU*mG*mC |
| 214 | mN*mN*mN*NNNNNNNNNNNNNNNNNGUUUUAGAmGmCmUmA(L1) |
| | mUmAmGmCAAGUUAAAAUAAGGC(L2)GUCCGUUAUCA(L1)GGCA |
| | CCGAGUCGG*mU*mG*mC |
| 215 | mN*mN*mN*NNNNNNNNNNNNNNNNNGUUUUAGAmGmCmU(L1)mA |
| | mGmCAAGUUAAAAUAAGGC(L2)GUCCGUUAUCAC(L1)GGGCACC |
| | GAGUCGG*mU*mG*mC |
| 216 | mN*mN*mN*NNNNNNNNNNNNNNNNNGUUUUAGAmGmCmU(L1)mA |
| | mGmCAAGUUAAAAUAAGGC(L2)GUCCGUUAUCA(L1)GGCACCGA |
| | GUCGG*mU*mG*mC |
| 217 | mN*mN*mN*NNNNNNNNNNNNNNNNNGUUUUAGAmGmCmU(L1)mA |
| | mGmCAAGUUAAAAUAAGGC(L2)GUCCGUUAUCA(L1)GGCACCGA |
| | GUCGG*mU*mG*mC |
| 218 | mN*mN*mN*NNNNNNNNNNNNNNNNNGUUUUAGAmGmCmU(L1)mA |
| | mGmCAAGUUAAAAUAAGGC(L2)GUCCGUUAUCA(L1)GGCACCGA |
| | GUCGG*mU*mG*mC |
| 219 | mN*mN*mN*NNNNNNNNNNNNNNNNNGUUUUAGAmGmCmUmA(L1) |
| | mUmAmGmCAAGUUAAAAUAAGGC(L2)GUCCGUUAUCAC(L1)GGG |
| | CACCGAGUCGG*mU*mG*mC |
| 220 | mN*mN*mN*NNNNNNNNNNNNNNNNNGUUUUAGAmGmCmUmA(L1) |
| | mUmAmGmCAAGUUAAAAUAAGGC(L2)GUCCGUUAUCA(L1)GGCA |
| | CCGAGUCGG*mU*mG*mC |
| 221 | mN*mN*mN*NNNNNNNNNNNNNNNNNGUUUUAGAmGmCmU(L1)mA |
| | mGmCAAGUUAAAAUAAGGC(L2)GUCCGUUAUCAC(L1)GGGCACC |
| | GAGUCGG*mU*mG*mC |
| 222 | mN*mN*mN*NNNNNNNNNNNNNNNNNGUUUUAGAmGmCmU(L1)mA |
| | mGmCAAGUUAAAAUAAGGC(L2)GUCCGUUAUCA(L1)GGCACCGA |
| | GUCGG*mU*mG*mC |
| 223 | mN*mN*mN*NNNNNNNNNNNNNNNNNGUUUUAGA(ds)AAGUUAAA |
| | AUAAGGCUAGUCCGUUAUCAC(L1)GGGCACCGAGUCGGmU*mG*m |
| | C*mU |
| 224 | mN*mN*mN*NNNNNNNNNNNNNNNNNGUUUUAGA(L4)AAGUUAAA |
| | AUAAGGCUAGUCCGUUAUCAC(L1)GGGCACCGAGUCGGmU*mG*m |
| | C*mU |
| 225 | mN*mN*mN*NNNNNNNNNNNNNNNNNGUUUUAGA(L3)AAGUUAAA |
| | AUAAGGCUAGUCCGUUAUCAC(L1)GGGCACCGAGUCGGmU*mG*m |
| | C*mU |
| 226 | mN*mN*mN*NNNNNNNNNNNNNNNNNGUUUUAGA(L2)AAGUUAAA |
| | AUAAGGCUAGUCCGUUAUCAC(L1)GGGCACCGAGUCGGmU*mG*m |
| | C*mU |
| 227 | mN*mN*mN*NNNNNNNNNNNNNNNNNGUUUUAGA(L1)AAGUUAAA |
| | AUAAGGCUAGUCCGUUAUCAC(L1)GGGCACCGAGUCGGmU*mG*m |
| | C*mU |
| 228 | mN*mN*mN*NNNNNNNNNNNNNNNNNGUUUUAGAmGmC(L1)mGmC |
| | AAGUUAAAAUAAGGCUAGUCCGUUAUCAC(L1)GGGCACCGAGUCG |
| | GmU*mG*mC*mU |
| 229 | mN*mN*mN*NNNNNNNNNNNNNNNNNGUUUUAGAmGmCmUmA(L1) |
| | mUmAmGmCAAGUUAAAAUAAGGCUAGUCCGUUAUCAC(L1)GGGCA |
| | CCGAGUCGGmU*mG*mC*mU |
| 230 | mN*mN*mN*NNNNNNNNNNNNNNNNNGUUUUAGAmGmCmUmA(L1) |
| | mUmAmGmCAAGUUAAAAUAAGGCUAGUCCGUUAUCAC(L1)GGGCA |
| | CCGAGUmCmGmGmU*mG*mC*mU |

Nucleotide modifications are indicated in Tables 2A-2C as follows: m: 2′-OMe; and *: PS linkage. As used herein, “N” may be any natural or non-natural nucleotide. For example, encompassed herein is SEQ ID NO: 230 in Table 2C, where the N's are replaced with any of the guide sequences disclosed herein. The modifications remain as shown in SEQ ID NO: 230 despite the substitution of N's for the nucleotides of a guide. That is, although the nucleotides of the guide replace the “N's”, the first three nucleotides are 2′-O-Me modified and there are phosphorothioate linkages between the first and second nucleotides, the second and third nucleotides and the third and fourth nucleotides.

E. Types of Chemical Modifications Described Herein

Guide RNAs (e.g., sgRNAs, dgRNAs, and crRNAs) comprising modifications at various positions are disclosed herein. In some embodiments, a position of a gRNA that comprises a modification is modified with any one or more of the following types of modifications.

2′-O-Methyl Modifications

Modified sugars are believed to control the puckering of nucleotide sugar rings, a physical property that influences oligonucleotide binding affinity for complementary strands, duplex formation, and interaction with nucleases. Substitutions on sugar rings can therefore alter the conformation and puckering of these sugars. For example, 2′-O-methyl (2′-OMe) modifications can increase binding affinity and nuclease stability of oligonucleotides, though as shown in the Examples, the effect of any modification at a given position in an oligonucleotide needs to be empirically determined.

The terms “mA,” “mC,” “mU,” or “mG” may be used to denote a nucleotide that has been modified with 2′-OMe.

A ribonucleotide and a modified 2′-O-methyl ribonucleotide can be depicted as follows:

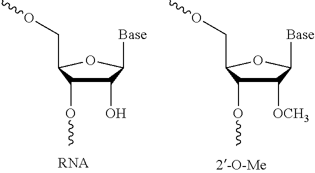

2′-O-(2-Methoxyethyl) Modifications

In some embodiments, the modification may be 2′-O-(2-methoxyethyl) (2′-O-moe). A modified 2′-O-moe ribonucleotide can be depicted as follows:

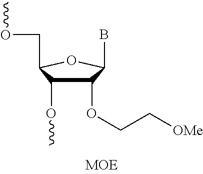

The terms “moeA,” “moeC,” “moeU,” or “moeG” may be used to denote a nucleotide that has been modified with 2′-O-moe.

2′-Fluoro Modifications

Another chemical modification that has been shown to influence nucleotide sugar rings is halogen substitution. For example, 2′-fluoro (2′-F) substitution on nucleotide sugar rings can increase oligonucleotide binding affinity and nuclease stability.

In this application, the terms “fA,” “fC,” “fJ,” or “fG” may be used to denote a nucleotide that has been substituted with 2′-F.

A ribonucleotide without and with a 2′-F substitution can be depicted as follows:

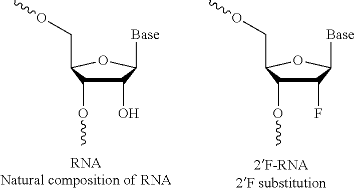

Phosphorothioate Modifications

A phosphorothioate (PS) linkage or bond refers to a bond where a sulfur is substituted for one nonbridging phosphate oxygen in a phosphodiester linkage, for example between nucleotides. When phosphorothioates are used to generate oligonucleotides, the modified oligonucleotides may also be referred to as S-oligos.

A “*” may be used to depict a PS modification. In this application, the terms A*, C*, U*, or G* may be used to denote a nucleotide that is linked to the next (e.g., 3′) nucleotide with a PS bond. Throughout this application, PS modifications are grouped with the nucleotide whose 3′ carbon is bonded to the phosphorothioate; thus, indicating that a PS modification is at position 1 means that the phosphorothioate is bonded to the 3′ carbon of nucleotide 1 and the 5′ carbon of nucleotide 2. Thus, where a YA site is indicated as being “PS modified” or the like, the PS linkage is between the Y and A or between the A and the next nucleotide.

In this application, the terms “mA*,” “mC*,” “mU*,” or “mG*” may be used to denote a nucleotide that has been substituted with 2′-OMe and that is linked to the next (e.g., 3′) nucleotide with a PS linkage, which may sometimes be referred to as a “PS bond.” Similarly, the terms “fA*,” “fC*,” “fU*,” or “fG*” may be used to denote a nucleotide that has been substituted with 2′-F and that is linked to the next (e.g., 3′) nucleotide with a PS linkage. Equivalents of a PS linkage or bond are encompassed by embodiments described herein.

The diagram below shows the substitution of S— for a nonbridging phosphate oxygen, generating a PS linkage in lieu of a phosphodiester linkage:

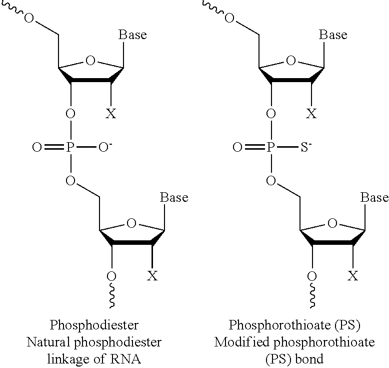

Inverted Abasic Modifications

Abasic nucleotides refer to those which lack nitrogenous bases. As abasic nucleotides cannot form a base pair, they do not disrupt formation of a structure by the unpaired nucleotides, e.g., a bulge, a loop. The figure below depicts an oligonucleotide with an abasic (in this case, shown as apurinic; an abasic site could also be an apyrimidinic site, wherein the description of the abasic site is typically in reference to Watson-Crick base pairing—e.g., an apurinic site refers to a site that lacks a nitrogenous base and would typically base pair with a pyrimidinic site) site that lacks a base, wherein the base may be substituted by another moiety at the 1′ position of the furan ring (e.g., a hydroxyl group, as shown below, to form a ribose or deoxyribose site, as shown below, or a hydrogen):

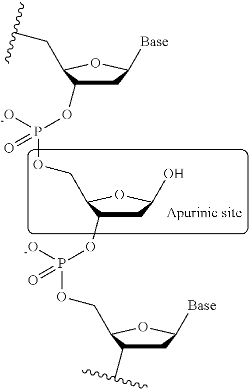

Inverted bases refer to those with linkages that are inverted from the normal 5′ to 3′ linkage (i.e., either a 5′ to 5′ linkage or a 3′ to 3′ linkage). For example:

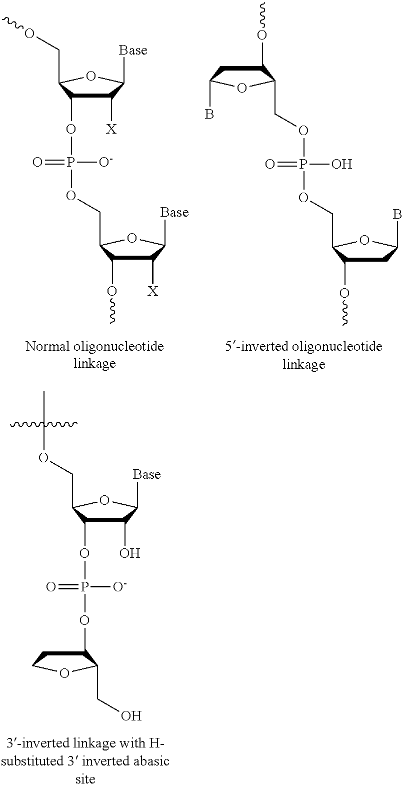

An abasic nucleotide can be attached with an inverted linkage. For example, an abasic nucleotide may be attached to the terminal 5′ nucleotide via a 5′ to 5′ linkage, or an abasic nucleotide may be attached to the terminal 3′ nucleotide via a 3′ to 3′ linkage. An inverted abasic nucleotide at either the terminal 5′ or 3′ nucleotide may also be called an inverted abasic end cap. In this application, the terms “invd” indicates an inverted abasic nucleotide linkage.

Deoxyribonucleotides

A deoxyribonucleotide (in which the sugar comprises a 2′-deoxy position) is considered a modification in the context of a gRNA, in that the nucleotide is modified relative to standard RNA by the substitution of a proton for a hydroxyl at the 2′ position. Unless otherwise indicated, a deoxyribonucleotide modification at a position that is U in an unmodified RNA can also comprise replacement of the U nucleobase with a T.

Bicyclic Ribose Analog

Exemplary bicyclic ribose analogs include locked nucleic acid (LNA), ENA, bridged nucleic acid (BNA), or another LNA-like modifications. In some instances, a bicyclic ribose analog has 2′ and 4′ positions connected through a linker. The linker can be of the formula —X—(CH₂)ₙ— where n is 1 or 2; X is O, NR, or S; and R is H or C₁₋₃ alkyl, e.g., methyl. Examples of bicyclic ribose analogs include LNAs comprising a 2′-O—CH₂-4′ bicyclic structure (oxy-LNA) (see WO 98/39352 and WO 99/14226); 2′-NH—CH₂-4′ or 2′-N(CH₃)—CH₂-4′ (amino-LNAs) (Singh et al., J. Org. Chem. 63:10035-10039 (1998); Singh et al., J. Org. Chem. 63:6078-6079 (1998)); and 2′-S—CH₂-4′ (thio-LNA) (Singh et al., J. Org. Chem. 63:6078-6079 (1998); Kumar et al., Biorg. Med. Chem. Lett. 8:2219-2222 (1998)). ENA

An ENA modification refers to a nucleotide comprising a 2′-O,4′-C-ethylene modification. An exemplary structure of an ENA nucleotide is shown below, in which wavy lines indicate connections to the adjacent nucleotides (or terminal positions as the case may be, with the understanding that if the 3′ terminal nucleotide is an ENA nucleotide, the 3′ position may comprise a hydroxyl rather than phosphate). For further discussion of ENA nucleotides, see, e.g., Koizumi et al., Nucleic Acids Res. 31: 3267-3273 (2003).

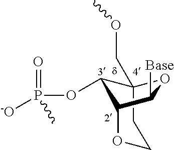

### UNA

A UNA or unlocked nucleic acid modification refers to a nucleotide comprising a 2′,3′-seco-RNA modification, in which the 2′ and 3′ carbons are not bonded directly to each other. An exemplary structure of a UNA nucleotide is shown below, in which wavy lines indicate connections to the adjacent phosphates or modifications replacing phosphates (or terminal positions as the case may be). For further discussion of UNA nucleotides, see, e.g., Snead et al., Molecular Therapy 2: e103, doi:10.1038/mtna.2013.36 (2013).

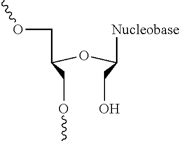

### Base Modifications

A base modification is any modification that alters the structure of a nucleobase or its bond to the backbone, including isomerization (as in pseudouridine). In some embodiments, a base modification includes inosine. In some embodiments, a modification comprises a base modification that reduces RNA endonuclease activity, e.g., by interfering with recognition of a cleavage site by an RNase or by stabilizing an RNA structure (e.g., secondary structure) that decreases accessibility of a cleavage site to an RNase. Exemplary base modifications that can stabilize RNA structures are pseudouridine and 5-methylcytosine. See Peacock et al., J Org Chem. 76: 7295-7300 (2011). In some embodiments, a base modification can increase or decrease the melting temperature (Tm) of a nucleic acid, e.g., by increasing the hydrogen bonding in a Watson-Crick base pair, forming non-canonical base pair, or creating a mismatched base pair.

The above modifications and their equivalents are included within the scope of the embodiments described herein.

### YA Modifications

A modification at a YA site (also referred to as a YA modification) can be a modification of the internucleoside linkage, a modification of the base (pyrimidine or adenine), e.g. by chemical modification, substitution, or otherwise, or a modification of the sugar (e.g. at the 2′ position, such as 2′-O-alkyl, 2′-F, 2′-moe, 2′-F arabinose, 2′-H (deoxyribose), and the like). In some embodiments, a “YA modification” is any modification that alters the structure of the dinucleotide motif to reduce RNA endonuclease activity, e.g., by interfering with recognition or cleavage of a YA site by an RNase or by stabilizing an RNA structure (e.g., secondary structure) that decreases accessibility of a cleavage site to an RNase. See Peacock et al., J Org Chem. 76: 7295-7300 (2011); Behlke, Oligonucleotides 18:305-320 (2008); Ku et al., Adv. Drug Delivery Reviews 104: 16-28 (2016); Ghidini et al., Chem. Commun., 2013, 49, 9036. Peacock et al., Belhke, Ku, and Ghidini provide exemplary modifications suitable as YA modifications. Modifications known to those of skill in the art to reduce endonucleolytic degradation are encompassed. Exemplary 2′ ribose modifications that affect the 2′ hydroxyl group involved in RNase cleavage are 2′-H and 2′-O-alkyl, including 2′-O-Me. Modifications such as bicyclic ribose analogs, UNA, and modified internucleoside linkages of the residues at the YA site can be YA modifications. Exemplary base modifications that can stabilize RNA structures are pseudouridine and 5-methylcytosine. In some embodiments, at least one nucleotide of the YA site is modified. In some embodiments, the pyrimidine (also called “pyrimidine position”) of the YA site comprises a modification (which includes a modification altering the internucleoside linkage immediately 3′ of the sugar of the pyrimidine, a modification of the pyrimidine base, and a modification of the ribose, e.g. at its 2′ position). In some embodiments, the adenine (also called “adenine position”) of the YA site comprises a modification (which includes a modification altering the internucleoside linkage immediately 3′ of the sugar of the pyrimidine, a modification of the pyrimidine base, and a modification of the ribose, e.g. at its 2′ position). In some embodiments, the pyrimidine and the adenine of the YA site comprise modifications. In some embodiments, the YA modification reduces RNA endonuclease activity.

The above modifications and their equivalents are included within the scope of the embodiments described herein.

Modifications of Guide Regions or YA Sites

In some embodiments, a gRNA comprises modifications at 1, 2, 3, 4, 5, 6, 7, 8, 9, 10, 11, 12, 13, 14, 15, 16, or more YA sites. In some embodiments, the pyrimidine of the YA site comprises a modification (which includes a modification altering the internucleoside linkage immediately 3′ of the sugar of the pyrimidine). In some embodiments, the adenine of the YA site comprises a modification (which includes a modification altering the internucleoside linkage immediately 3′ of the sugar of the adenine). In some embodiments, the pyrimidine and the adenine of the YA site comprise modifications, such as sugar, base, or internucleoside linkage modifications. The YA modifications can be any of the types of modifications set forth herein. In some embodiments, the YA modifications comprise one or more of phosphorothioate, 2′-OMe, or 2′-fluoro. In some embodiments, the YA modifications comprise pyrimidine modifications comprising one or more of phosphorothioate, 2′-OMe, 2′-H, inosine, or 2′-fluoro. In some embodiments, the YA modification comprises a bicyclic ribose analog (e.g., an LNA, BNA, or ENA) within an RNA duplex region that contains one or more YA sites. In some embodiments, the YA modification comprises a bicyclic ribose analog (e.g., an LNA, BNA, or ENA) within an RNA duplex region that contains a YA site, wherein the YA modification is distal to the YA site.

The guide region of a gRNA may be modified according to any embodiment comprising a modified guide region set forth herein. In some embodiments, the guide region comprises 1, 2, 3, 4, 5, or more YA sites (“guide region YA sites”) that may comprise YA modifications. In some embodiments, the modified guide region YA sites comprise modifications as described for YA sites above. Additional embodiments of guide region modifications, including guide region YA site modifications, are set forth elsewhere herein. Any embodiments set forth elsewhere in this disclosure may be combined to the extent feasible with any of the foregoing embodiments.

Modifications to Terminal Nucleotides

In some embodiments, the 5′ or 3′ terminus regions of a gRNA are modified.

3′ Terminus Region Modifications

In some embodiments, the terminal (i.e., last) 1, 2, 3, 4, 5, 6, or 7 nucleotides in the 3′ terminus region are modified. Throughout, this modification may be referred to as a “3′ end modification”. In some embodiments, the terminal (i.e., last) 1, 2, 3, 4, 5, 6, or 7 nucleotides in the 3′ terminus region comprise more than one modification. In some embodiments, at least one of the terminal (i.e., last) 1, 2, 3, 4, 5, 6, or 7 nucleotides in the 3′ terminus region are modified. In some embodiments, at least two of the terminal (i.e., last) 1, 2, 3, 4, 5, 6, or 7 nucleotides in the 3′ terminus region are modified. In some embodiments, at least three of the terminal (i.e., last) 1, 2, 3, 4, 5, 6, or 7 nucleotides in the 3′ terminus region are modified. In some embodiments, the modification comprises a PS linkage. In some embodiments, the modification to the 3′ terminus region is a 3′ protective end modification. In some embodiments, the 3′ end modification comprises a 3′ protective end modification.

In some embodiments, the 3′ end modification comprises a modified nucleotide selected from 2′-O-methyl (2′-O-Me) modified nucleotide, 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide, or combinations thereof.

In some embodiments, the 3′ end modification comprises or further comprises a 2′-O-methyl (2′-O-Me) modified nucleotide.

In some embodiments, the 3′ end modification comprises or further comprises a 2′-fluoro (2′-F) modified nucleotide.

In some embodiments, the 3′ end modification comprises or further comprises a phosphorothioate (PS) linkage between nucleotides.

In some embodiments, the 3′ end modification comprises or further comprises an inverted abasic modified nucleotide.

In some embodiments, the 3′ end modification comprises or further comprises a modification of any one or more of the last 7, 6, 5, 4, 3, 2, or 1 nucleotides. In some embodiments, the 3′ end modification comprises or further comprises one modified nucleotide. In some embodiments, the 3′ end modification comprises or further comprises two modified nucleotides. In some embodiments, the 3′ end modification comprises or further comprises three modified nucleotides. In some embodiments, the 3′ end modification comprises or further comprises four modified nucleotides. In some embodiments, the 3′ end modification comprises or further comprises five modified nucleotides. In some embodiments, the 3′ end modification comprises or further comprises six modified nucleotides. In some embodiments, the 3′ end modification comprises or further comprises seven modified nucleotides.

In some embodiments, the 3′ end modification comprises or further comprises a modification of between 1 and 7 or between 1 and 5 nucleotides.

In some embodiments, the 3′ end modification comprises or further comprises modifications of 1, 2, 3, 4, 5, 6, or 7 nucleotides at the 3′ end of the gRNA.

In some embodiments, the 3′ end modification comprises or further comprises modifications of about 1-3, 1-5, 1-6, or 1-7 nucleotides at the 3′ end of the gRNA.

In some embodiments, the 3′ end modification comprises or further comprises any one or more of the following: a phosphorothioate (PS) linkage between nucleotides, a 2′-O-Me modified nucleotide, a 2′-O-moe modified nucleotide, a 2′-F modified nucleotide, an inverted abasic modified nucleotide, and a combination thereof.

In some embodiments, the 3′ end modification comprises or further comprises 1, 2, 3, 4, 5, 6, or 7 PS linkages between nucleotides.

In some embodiments, the 3′ end modification comprises or further comprises at least one 2′-O-Me, 2′-O-moe, inverted abasic, or 2′-F modified nucleotide. In some embodiments, the 3′ end modification comprises or further comprises one PS linkage, wherein the linkage is between the last and second to last nucleotide. In some embodiments, the 3′ end modification comprises or further comprises two PS linkages between the last three nucleotides. In some embodiments, the 3′ end modification comprises or further comprises four PS linkages between the last four nucleotides.

In some embodiments, the 3′ end modification comprises or further comprises PS linkages between any one or more of the last four nucleotides. In some embodiments, the 3′ end modification comprises or further comprises PS linkages between any one or more of the last five nucleotides. In some embodiments, the 3′ end modification comprises or further comprises PS linkages between any one or more of the last 2, 3, 4, 5, 6, or 7 nucleotides.

In some embodiments, the 3′ end modification comprises or further comprises a modification of one or more of the last 1-7 nucleotides, wherein the modification is a PS linkage, inverted abasic nucleotide, 2′-OMe, 2′-O-moe, 2′-F, or combinations thereof.

In some embodiments, the 3′ end modification comprises or further comprises a modification to the last nucleotide with 2′-OMe, 2′-O-moe, 2′-F, or combinations thereof, and an optionally one or two PS linkages to the next nucleotide or the first nucleotide of the 3′ tail.

In some embodiments, the 3′ end modification comprises or further comprises a modification to the last or second to last nucleotide with 2′-OMe, 2′-O-moe, 2′-F, or combinations thereof, and optionally one or more PS linkages.

In some embodiments, the 3′ end modification comprises or further comprises a modification to the last, second to last, or third to last nucleotides with 2′-OMe, 2′-O-moe, 2′-F, or combinations thereof, and optionally one or more PS linkages.

In some embodiments, the 3′ end modification comprises or further comprises a modification to the last, second to last, third to last, or fourth to last nucleotides with 2′-OMe, 2′-O-moe, 2′-F, or combinations thereof, and optionally one or more PS linkages.

In some embodiments, the 3′ end modification comprises or further comprises a modification to the last, second to last, third to last, fourth to last, or fifth to last nucleotides with 2′-OMe, 2′-O-moe, 2′-F, or combinations thereof, and optionally one or more PS linkages.

In certain embodiments, the 3′ end modification comprises 2′-O-Me modifications and PS modifications. In some embodiments, the 3′ end modification comprises the same number of 2′-O-Me modifications and PS modifications. In some embodiments, the 3′ end modification comprises one more 2′-O-Me modification than PS modification. In some embodiments, the 3′ end modification comprises one fewer 2′-O-Me modification than PS modification. In certain embodiments, the 3′ end modification comprises 4 2′-O-Me modifications. In certain embodiments, the 3′ end modification comprises 3 2′-O-Me modifications.

In some embodiments, the gRNA comprising a 3′ end modification comprises or further comprises a 3′ tail, wherein the 3′ tail comprises a modification of any one or more of the nucleotides present in the 3′ tail. In some embodiments, the 3′ tail is fully modified. In some embodiments, the 3′ tail comprises 1, 2, 3, 4, 5, 6, 7, 8, 9, 10, 1-2, 1-3, 1-4, 1-5, 1-6, 1-7, 1-8, 1-9, or 1-10 nucleotides, optionally where any one or more of these nucleotides are modified.

3′ Tail

In some embodiments, the gRNA comprises a 3′ terminus comprising a 3′ tail, which follows and is 3′ of the conserved portion of a gRNA. In some embodiments, the 3′ tail comprises between 1 and about 20 nucleotides, between 1 and about 15 nucleotides, between 1 and about 10 nucleotides, between 1 and about 5 nucleotides, between 1 and about 4 nucleotides, between 1 and about 3 nucleotides, and between 1 and about 2 nucleotides. In some embodiments, the 3′ tail comprises about 1, 2, 3, 4, 5, 6, 7, 8, 9, or 10 nucleotides. In some embodiments, the 3′ tail comprises 1, 2, 3, 4, 5, 6, 7, 8, 9, or 10 nucleotides. In some embodiments, the 3′ tail comprises 1 nucleotide. In some embodiments, the 3′ tail comprises 2 nucleotides. In some embodiments, the 3′ tail comprises 3 nucleotides. In some embodiments, the 3′ tail comprises 4 nucleotides. In some embodiments, the 3′ tail comprises about 1-2, 1-3, 1-4, 1-5, 1-7, 1-10, at least 1-5, at least 1-3, at least 1-4, at least 1-5, at least 1-5, at least 1-7, or at least 1-10 nucleotides. In some embodiments, the tail terminates with a nucleotide comprising a uracil or a modified uracil. In some embodiments, the 3′ tail is 1 nucleotide in length and is a nucleotide comprising a uracil or a modified uracil. In some embodiments, the 3′ nucleotide of the gRNA is a nucleotide comprising a uracil or a modified uracil.

In some embodiments, the 3′ tail comprising 1-20 nucleotides and follows the 3′ end of the conserved portion of a gRNA.

In some embodiments, the 3′ tail comprises or further comprises one or more of a protective end modification, a phosphorothioate (PS) linkage between nucleotides, a 2′-OMe modified nucleotide, a 2′-O-moe modified nucleotide, a 2′-F modified nucleotide, an inverted abasic modified nucleotide, and a combination thereof.

In some embodiments, the 3′ tail comprises or further comprises one or more phosphorothioate (PS) linkages between nucleotides. In some embodiments, the 3′ tail comprises or further comprises one or more 2′-OMe modified nucleotides. In some embodiments, the 3′ tail comprises or further comprises one or more 2′-O-moe modified nucleotides. In some embodiments, the 3′ tail comprises or further comprises one or more 2′-F modified nucleotide. In some embodiments, the 3′ tail comprises or further comprises one or more an inverted abasic modified nucleotides. In some embodiments, the 3′ tail comprises or further comprises one or more protective end modifications. In some embodiments, the 3′ tail comprises or further comprises a combination of one or more of a phosphorothioate (PS) linkage between nucleotides, a 2′-OMe modified nucleotide, a 2′-O-moe modified nucleotide, a 2′-F modified nucleotide, and an inverted abasic modified nucleotide.

In some embodiments, the gRNA does not comprise a 3′ tail.

5′ Terminus Region Modifications

In some embodiments, the 5′ terminus region is modified, for example, the first 1, 2, 3, 4, 5, 6, or 7 nucleotides of the gRNA are modified. Throughout, this modification may be referred to as a “5′ end modification”. In some embodiments, the first 1, 2, 3, 4, 5, 6, or 7 nucleotides of the 5′ terminus region comprise more than one modification. In some embodiments, at least one of the terminal (i.e., first) 1, 2, 3, 4, 5, 6, or 7 nucleotides at the 5′ end are modified. In some embodiments, at least two of the terminal 1, 2, 3, 4, 5, 6, or 7 nucleotides at the 5′ terminus region are modified. In some embodiments, at least three of the terminal 1, 2, 3, 4, 5, 6, or 7 nucleotides at the 5′ terminus region are modified. In some embodiments, the 5′ end modification is a 5′ protective end modification.

In some embodiments, both the 5′ and 3′ terminus regions (e.g., ends) of the gRNA are modified. In some embodiments, only the 5′ terminus region of the gRNA is modified. In some embodiments, only the 3′ terminus region (plus or minus a 3′ tail) of the conserved portion of a gRNA is modified.

In some embodiments, the gRNA comprises modifications at 1, 2, 3, 4, 5, 6, or 7 of the first 7 nucleotides at a 5′ terminus region of the gRNA. In some embodiments, the gRNA comprises modifications at 1, 2, 3, 4, 5, 6, or 7 of the 7 terminal nucleotides at a 3′ terminus region. In some embodiments, 2, 3, or 4 of the first 4 nucleotides at the 5′ terminus region, or 2, 3, or 4 of the terminal 4 nucleotides at the 3′ terminus region are modified. In some embodiments, 2, 3, or 4 of the first 4 nucleotides at the 5′ terminus region are linked with phosphorothioate (PS) bonds.

In some embodiments, the modification to the 5′ terminus or 3′ terminus comprises a 2′-O-methyl (2′-O-Me) or 2′-O-(2-methoxyethyl) (2′-O-moe) modification. In some embodiments, the modification comprises a 2′-fluoro (2′-F) modification to a nucleotide. In some embodiments, the modification comprises a phosphorothioate (PS) linkage between nucleotides. In some embodiments, the modification comprises an inverted abasic nucleotide. In some embodiments, the modification comprises a protective end modification. In some embodiments, the modification comprises a more than one modification selected from protective end modification, 2′-O-Me, 2′-O-moe, 2′-fluoro (2′-F), a phosphorothioate (PS) linkage between nucleotides, and an inverted abasic nucleotide. In some embodiments, an equivalent modification is encompassed.

In some embodiments, the gRNA comprises one or more phosphorothioate (PS) linkages between the first one, two, three, four, five, six, or seven nucleotides at the 5′ terminus. In some embodiments, the gRNA comprises one or more PS linkages between the last one, two, three, four, five, six, or seven nucleotides at the 3′ terminus. In some embodiments, the gRNA comprises one or more PS linkages between both the last one, two, three, four, five, six, or seven nucleotides at the 3′ terminus and the first one, two, three, four, five, six, or seven nucleotides from the 5′ end of the 5′ terminus. In some embodiments, in addition to PS linkages, the 5′ and 3′ terminal nucleotides may comprise 2′-O-Me, 2′-O-moe, or 2′-F modified nucleotides.

In some embodiments, the gRNA comprises a 5′ end modification, e.g., wherein the first nucleotide of the guide region is modified. In some embodiments, the gRNA comprises a 5′ end modification, wherein the first nucleotide of the guide region comprises a 5′ protective end modification.

In some embodiments, the 5′ end modification comprises a modified nucleotide selected from 2′-O-methyl (2′-O-Me) modified nucleotide, 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide, or combinations thereof.

In some embodiments, the 5′ end modification comprises or further comprises a 2′-O-methyl (2′-O-Me) modified nucleotide.

In some embodiments, the 5′ end modification comprises or further comprises a 2′-fluoro (2′-F) modified nucleotide.

In some embodiments, the 5′ end modification comprises or further comprises a phosphorothioate (PS) linkage between nucleotides.

In some embodiments, the 5′ end modification comprises or further comprises an inverted abasic modified nucleotide.

In some embodiments, the 5′ end modification comprises or further comprises a modification of any one or more of nucleotides 1-7 of the guide region of a gRNA. In some embodiments, the 5′ end modification comprises or further comprises one modified nucleotide. In some embodiments, the 5′ end modification comprises or further comprises two modified nucleotides. In some embodiments, the 5′ end modification comprises or further comprises three modified nucleotides. In some embodiments, the 5′ end modification comprises or further comprises four modified nucleotides. In some embodiments, the 5′ end modification comprises or further comprises five modified nucleotides. In some embodiments, the 5′ end modification comprises or further comprises six modified nucleotides. In some embodiments, the 5′ end modification comprises or further comprises seven modified nucleotides.

In some embodiments, the 5′ end modification comprises or further comprises a modification of between 1 and 7, between 1 and 5, between 1 and 4, between 1 and 3, or between 1 and 2 nucleotides.

In some embodiments, the 5′ end modification comprises or further comprises modifications of 1, 2, 3, 4, 5, 6, or 7 nucleotides from the 5′ end. In some embodiments, the 5′ end modification comprises or further comprises modifications of about 1-3, 1-4, 1-5, 1-6, or 1-7 nucleotides from the 5′ end.

In some embodiments, the 5′ end modification comprises or further comprises modifications at the first nucleotide at the 5′ end of the gRNA. In some embodiments, the 5′ end modification comprises or further comprises modifications at the first and second nucleotide from the 5′ end of the gRNA. In some embodiments, the 5′ end modification comprises or further comprises modifications at the first, second, and third nucleotide from the 5′ end of the gRNA. In some embodiments, the 5′ end modification comprises or further comprises modifications at the first, second, third, and fourth nucleotide from the 5′ end of the gRNA. In some embodiments, the 5′ end modification comprises or further comprises modifications at the first, second, third, fourth, and fifth nucleotide from the 5′ end of the gRNA. In some embodiments, the 5′ end modification comprises or further comprises modifications at the first, second, third, fourth, fifth, and sixth nucleotide from the 5′ end of the gRNA. In some embodiments, the 5′ end modification comprises or further comprises modifications at the first, second, third, fourth, fifth, sixth, and seventh nucleotide from the 5′ end of the gRNA.

In some embodiments, the 5′ end modification comprises or further comprises a phosphorothioate (PS) linkage between nucleotides, or a 2′-O-Me modified nucleotide, or a 2′-O-moe modified nucleotide, or a 2′-F modified nucleotide, or an inverted abasic modified nucleotide, or combinations thereof.

In some embodiments, the 5′ end modification comprises or further comprises 1, 2, 3, 4, 5, 6, or 7 PS linkages between nucleotides. In some embodiments, the 5′ end modification comprises or further comprises about 1-2, 1-3, 1-4, 1-5, 1-6, or 1-7 PS linkages between nucleotides.

In some embodiments, the 5′ end modification comprises or further comprises at least one PS linkage, wherein if there is one PS linkage, the linkage is between nucleotides 1 and 2 of the guide region.

In some embodiments, the 5′ end modification comprises or further comprises at least two PS linkages, and the linkages are between nucleotides 1 and 2, and 2 and 3 of the guide region.

In some embodiments, the 5′ end modification comprises or further comprises PS linkages between any one or more of nucleotides 1 and 2, 2 and 3, and 3 and 4 of the guide region.

In some embodiments, the 5′ end modification comprises or further comprises PS linkages between any one or more of nucleotides 1 and 2, 2 and 3, 3 and 4, and 4 and 5 of the guide region.

In some embodiments, the 5′ end modification comprises or further comprises PS linkages between any one or more of nucleotides 1 and 2, 2 and 3, 3 and 4, 4 and 5, and 5 and 6 of the guide region.

In some embodiments, the 5′ end modification comprises or further comprises PS linkages between any one or more of nucleotides 1 and 2, 2 and 3, 3 and 4, 4 and 5, 5 and 6, and 7 and 8 of the guide region.

In some embodiments, the 5′ end modification comprises or further comprises a modification of one or more of nucleotides 1-7 of the guide region, wherein the modification is a PS linkage, inverted abasic nucleotide, 2′-O-Me, 2′-O-moe, 2′-F, or combinations thereof.

In some embodiments, the 5′ end modification comprises or further comprises a modification to the first nucleotide of the guide region with 2′-O-Me, 2′-O-moe, 2′-F, or combinations thereof, and an optional PS linkage to the next nucleotide;

In some embodiments, the 5′ end modification comprises or further comprises a modification to the first or second nucleotide of the guide region with 2′-O-Me, 2′-O-moe, 2′-F, or combinations thereof, and optionally one or more PS linkages between the first and second nucleotide or between the second and third nucleotide.

In some embodiments, the 5′ end modification comprises or further comprises a modification to the first, second, or third nucleotides of the variable region with 2′-O-Me, 2′-O-moe, 2′-F, or combinations thereof, and optionally one or more PS linkages between the first and second nucleotide, between the second and third nucleotide, or between the third and the fourth nucleotide.

In some embodiments, the 5′ end modification comprises or further comprises a modification to the first, second, third, or fourth nucleotides of the variable region with 2′-O-Me, 2′-O-moe, 2′-F, or combinations thereof, and optionally one or more PS linkages between the first and second nucleotide, between the second and third nucleotide, between the third and the fourth nucleotide, or between the fourth and the fifth nucleotide.

In some embodiments, the 5′ end modification comprises or further comprises a modification to the first, second, third, fourth, or fifth nucleotides of the variable region with 2′-O-Me, 2′-O-moe, 2′-F, or combinations thereof, and optionally one or more PS linkages between the first and second nucleotide, between the second and third nucleotide, between the third and the fourth nucleotide, between the fourth and the fifth nucleotide, or between the fifth and the sixth nucleotide.

Repeat-Anti-Repeat Region Modifications

In some embodiments, a gRNA is provided comprising a repeat-anti-repeat region modification, wherein the repeat-anti-repeat region modification comprises a modification of at least 1, 2, 3, 4, 5, 6, 7, 8, 9, 10, 11, or all 12 nucleotides in the repeat-anti-repeat region.

In some embodiments, a gRNA is provided comprising a repeat-anti-repeat region modification, wherein the repeat-anti-repeat region modification comprises a modification of about 1-2, 1-3, 1-4, 1-5, 1-6, 1-7, 1-8, 1-9, 1-10, or 1-12 nucleotides in the repeat-anti-repeat region region.

In some embodiments, a gRNA is provided comprising a repeat-anti-repeat region modification, wherein the upper stem modification comprises a 2′-OMe modified nucleotide. In some embodiments, a gRNA is provided comprising a repeat-anti-repeat region modification, wherein the upper stem modification comprises a 2′-O-moe modified nucleotide. In some embodiments, a gRNA is provided comprising a repeat-anti-repeat region modification, wherein the upper stem modification comprises a 2′-F modified nucleotide.

In some embodiments, a gRNA is provided comprising a repeat-anti-repeat region modification, wherein the repeat-anti-repeat region modification comprises a 2′-OMe modified nucleotide, a 2′-O-moe modified nucleotide, a 2′-F modified nucleotide, or combinations thereof.

In some embodiments, the gRNA comprises a 5′ end modification and a repeat-anti-repeat region modification. In some embodiments, the gRNA comprises a 3′ end modification and a repeat-anti-repeat region modification. In some embodiments, the gRNA comprises a 5′ end modification, a 3′ end modification and an upper stem modification.

Hairpin Modifications

In some embodiments, the gRNA comprises a modification in the hairpin region. In some embodiments, the hairpin region modification comprises at least one modified nucleotide selected from a 2′-O-methyl (2′-OMe) modified nucleotide, a 2′-fluoro (2′-F) modified nucleotide, or combinations thereof.

In some embodiments, the hairpin region modification is in the hairpin 1 region. In some embodiments, the hairpin region modification is in the hairpin 2 region. In some embodiments, modifications are within the hairpin 1 and hairpin 2 regions, optionally wherein the “n” between hairpin 1 and 2 is also modified.

In some embodiments, the hairpin modification comprises or further comprises a 2′-O-methyl (2′-OMe) modified nucleotide.

In some embodiments, the hairpin modification comprises or further comprises a 2′-fluoro (2′-F) modified nucleotide.

In some embodiments, the hairpin region modification comprises at least one modified nucleotide selected from a 2′H modified nucleotide (DNA), PS modified nucleotide, a YA modification, a 2′-O-methyl (2′-O-Me) modified nucleotide, a 2′-fluoro (2′-F) modified nucleotide, or combinations thereof.

In some embodiments, the gRNA comprises a 3′ end modification, and a modification in the hairpin region.

In some embodiments, the gRNA comprises a 5′ end modification, and a modification in the hairpin region.

In some embodiments, the gRNA comprises an upper stem modification, and a modification in the hairpin region.

In some embodiments, the gRNA comprises a 3′ end modification, a modification in the hairpin region, an upper stem modification, and a 5′ end modification.

F. Exemplary Modified Guide RNAS

Modified gRNAs comprising combinations of 5′ end modifications, 3′ end modifications, upper stem modifications, hairpin modifications, and 3′ terminus modifications, as described above, are encompassed. Exemplary modified gRNAs are described below.

sgRNAs; Domains/Regions Thereof

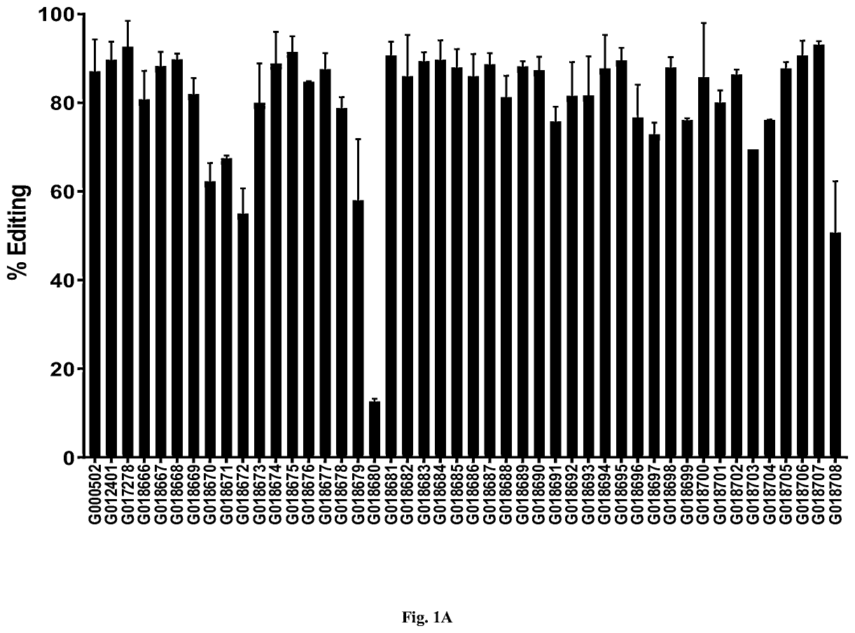

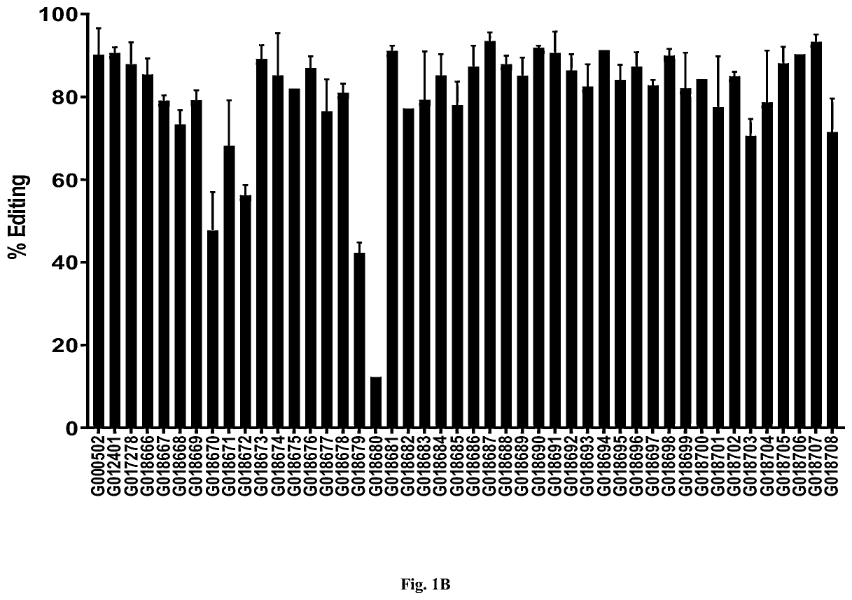

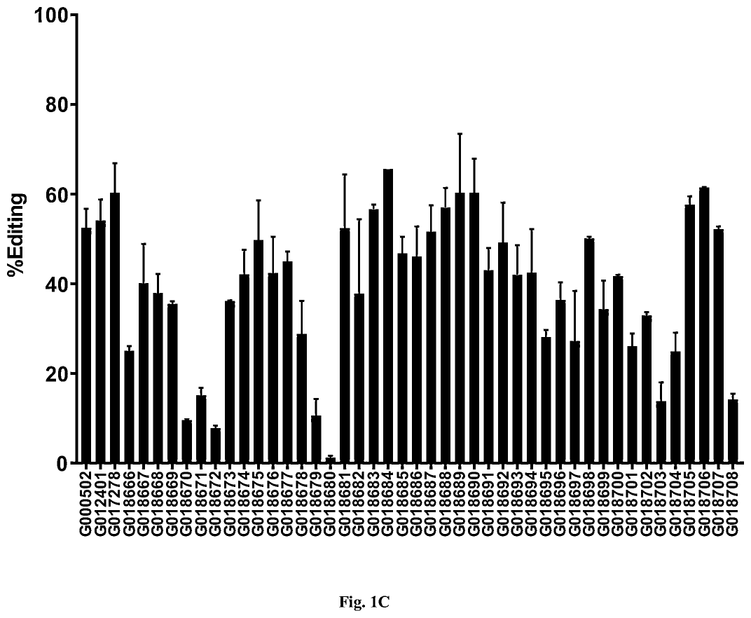

In some embodiments, a gRNA provided herein is an sgRNA. Briner A E et al., Molecular Cell 56:333-339 (2014) describes functional domains of sgRNAs, referred to herein as “domains”, including the “spacer” domain responsible for targeting, the “lower stem”, the “bulge”, “upper stem” (which may include a tetraloop), the “nexus”, and the “hairpin 1” and “hairpin 2” domains. See Briner et al. at page 334, FIG. 1A. As described in detail elsewhere herein, one or more domains (e.g., hairpin 1 or the upper stem) may be shortened in an sgRNA described herein.

In some embodiments, the sgRNA comprises a guide region and a conserved portion 3′ to the guide region, wherein the conserved portion comprises a repeat-anti-repeat region, a nexus region, a hairpin 1 region, and a hairpin 2 region. The repeat-anti-repeat region comprises an upper stem region and a lower stem region. Table 3B provides a schematic of the domains of an sgRNA as used herein. In Table 3B, the “n” between regions represents a variable number of nucleotides, for example, from 0 to 1, 2, 3, 4, 5, 6, 7, 8, 9, 10, 11, 12, 13, 14, 15, 16, 17, 18, 19, 20, or more. In some embodiments, n equals 0. In some embodiments, n equals 1.

In some embodiments, the sgRNA comprises at least one of: a first internal linker substituting for at least 4 nucleotides of the upper stem region; a second internal linker substituting for 2 nucleotides of the nexus region; and a third internal linker substituting for at least 2 nucleotides of the hairpin 1.

In some embodiments, the sgRNA comprises the first internal linker and the second internal linker. In some embodiments, the sgRNA comprises the first internal linker and the third internal linker. In some embodiments, the sgRNA comprises the second internal linker and the second internal linker. In some embodiments, the sgRNA comprise the first internal linker, the second internal linker, and the second internal linker.

In some embodiments, the first internal linker has a bridging length of about 9-30 atoms, optionally about 15-21 atoms. In some embodiments, the first internal linker substitutes for 4, 5, 6, 7, 8, 9, 10, 11, or 12 nucleotides of the repeat-anti-repeat region of the gRNA.

In some embodiments, the second internal linker has a bridging length of about 9-15 atoms. In some embodiments, the second internal linker substitutes for a hairpin region of the nexus region of the sgRNA. In some embodiments, the second internal linker substitutes for 2 nucleotides of a stem region of the nexus region of the sgRNA.

In some embodiments, the third internal linker has a bridging length of about 9-30 atoms, optionally about 15-21 atoms. In some embodiments, the third internal linker substitutes for 4, 5, 6, 7, 8, 9. 10, 11, or 12 nucleotides of the hairpin 1 of the gRNA.

In some embodiments, the first internal linker is in a hairpin between a first portion and a second portion, and the first portion and the second portion together form a duplex portion.

In some embodiments, the third internal linker is in a hairpin between a first portion of the sgRNA and second portion of the sgRNA, and the first portion and the second portion together form a duplex portion.

In some embodiments, a hairpin 2 region of the sgRNA does not contain any internal linker. In some embodiments, the hairpin 2 region is in a SpyCas9 gRNA.

5′ Terminus Region

In some embodiments, the sgRNA comprises nucleotides at the 5′ end as shown in Table 3A-B. In some embodiments, the 5′ terminus of the sgRNA comprises a spacer or guide region that functions to direct a Cas protein, e.g., a Cas9 protein, to a target nucleotide sequence. In some embodiments, the 5′ terminus does not comprise a guide region. In some embodiments, the 5′ terminus comprises a spacer and additional nucleotides that do not function to direct a Cas protein to a target nucleotide region.

Lower Stem

In some embodiments, the sgRNA comprises a lower stem (LS) region that when viewed linearly, is separated by a bulge and upper stem regions. See Table 3A-B.

In some embodiments, the lower stem regions comprise 1-12 nucleotides, e.g. in one embodiment the lower stem regions comprise LS1-LS12. In some embodiments, the lower stem region comprises fewer nucleotides than shown in Table 3. In some embodiments, the lower stem region comprises more nucleotides than shown in Table 3A-B. When the lower stem region comprises fewer or more nucleotides than shown in the schematic of Table 3, the modification pattern, as will be apparent to the skilled artisan, should be maintained.

In some embodiments, the lower stem region has nucleotides that are complementary in nucleic acid sequence when read in opposite directions. In some embodiments, the complementarity in nucleic acid sequence of lower stem leads to a secondary structure of a stem in the sgRNA (e.g., the regions may base pair with one another). In some embodiments, the lower stem regions may not be perfectly complimentary to each other when read in opposite directions.

Bulge

In some embodiments, the sgRNA comprises a bulge region comprising six nucleotides, B1-B6. When viewed linearly, the bulge region is separated into two regions. See Table 3. In some embodiments, the bulge region comprises six nucleotides, wherein the first two nucleotides are followed by an upper stem region, followed by the last four nucleotides of the bulge. In some embodiments, the bulge region comprises fewer nucleotides than shown in Table 3A-B. In some embodiments, the bulge region comprises more nucleotides than shown in Table 3A-B. When the bulge region comprises fewer or more nucleotides than shown in the schematic of Table 3A-B, the modification pattern, as will be apparent to the skilled artisan, should be maintained.

In some embodiments, the presence of a bulge results in a directional kink between the upper and lower stem modules in an sgRNA.

Upper Stem

In some embodiments, the upper stem region is a shortened upper stem region, such as any of the shortened upper stem regions described elsewhere herein.

In other embodiments, the sgRNA comprises an upper stem region comprising 12 nucleotides. In some embodiments, the upper stem region comprises a loop sequence. In some instances, the loop is a tetraloop (loop consisting of four nucleotides). In some embodiments, the upper stem region comprises more nucleotides than shown in Table 3B.

When the upper stem region comprises fewer or more nucleotides than shown in the schematic of Table 3A-B, the modification pattern, as will be apparent to the skilled artisan, should be maintained.

In some embodiments, the upper stem region has nucleotides that are complementary in nucleic acid sequence when read in opposite directions. In some embodiments, the complementarity in nucleic acid sequence of upper stem leads to a secondary structure of a stem in the sgRNA (e.g., the regions may base pair with one another). In some embodiments, the upper stem regions may not be perfectly complimentary to each other when read in opposite directions.

In some embodiments, the upper stem region comprises fewer nucleotides than shown in FIG. 10A, and sometimes is not present. In certain embodiments, bulge nucleotides B2 and B3 (corresponding to nucleotides 8 and 21 of SEQ ID: 400; see Table 3A) are directly joined (i.e., such that no intervening nucleotides are present) by an internal linker. In certain embodiments, B2 and B3 are directly joined by one or more, e.g., 1, 2, 3, or 4 abasic nucleosides. In certain embodiments, B2 and B3 are joined by an internal linker or one or more, e.g., 1, 2, 3, or 4, abasic nucleosides wherein additional nucleotides present do not form a duplex portion above the bulge. In certain embodiments, B2 and B3 are joined by an internal linker or one or more, e.g., 1, 2, 3, or 4 abasic nucleoside wherein additional nucleotides present do not form a duplex portion longer than 3 nucleotides above the bulge.

Nexus

In some embodiments, the sgRNA comprises a nexus region that is located between the lower stem region and the hairpin 1 region. In some embodiments, the nexus comprises 18 nucleotides. In some embodiments, the nexus region comprises nucleotides N1 through N18 as shown in Table 3A-B. In some embodiments, the nexus region comprises a substitution (e.g., at position N18) or lacks a nucleotide, such as any of the nexus regions with a substitution or lacking a nucleotide described in detail elsewhere herein.

In some embodiments, the nexus region comprises fewer nucleotides than shown in Table 3A-B. In some embodiments, the nexus region comprises more nucleotides than shown in Table 3A-B. When the nexus region comprises fewer or more nucleotides than shown in the schematic of Table 3A-B, the modification pattern, as will be apparent to the skilled artisan, should be maintained.

In some embodiments, the nexus region has nucleotides that are complementary in nucleic acid sequence when read in opposite directions. In some embodiments, the complementarity in nucleic acid sequence leads to a secondary structure of a stem or stem loop in the sgRNA (e.g., certain nucleotides in the nexus region may base pair with one another). In some embodiments, the nexus regions may not be perfectly complimentary to each other when read in opposite directions.

Hairpin

In some embodiments, the sgRNA comprises one or more hairpin structures within the hairpin region. The hairpin region is downstream of (i.e., 3′ to) the repeat-anti-repeat region. In some embodiments, the hairpin region is downstream of the nexus region, when present. In some embodiments, the region of nucleotides immediately downstream of the nexus region is termed “hairpin 1” or “H1”. In some embodiments, the region of nucleotides 3′ to hairpin 1 is termed “hairpin 2” or “H2”. In some embodiments, the hairpin region comprises both hairpin 1 and hairpin 2. In some embodiments, the sgRNA comprises hairpin 1 or hairpin 2.

In some embodiments, the hairpin 1 region is a shortened hairpin 1 region, such as any of the shortened hairpin 1 regions described elsewhere herein.

In other embodiments, the hairpin 1 region comprises 12 nucleotides immediately downstream of the nexus region. In some embodiments, the hairpin 1 region comprises nucleotides H1-1 through H1-12 as shown in Table 3B.

In some embodiments, the hairpin 2 region comprises 15 nucleotides downstream of the hairpin 1 region. In some embodiments, the hairpin 2 region comprises nucleotides H2-1 through H2-15 as shown in Table 3B.

In some embodiments, one or more nucleotides is present between the hairpin 1 and the hairpin 2 regions. The one or more nucleotides between the hairpin 1 and hairpin 2 region may be modified or unmodified. In some embodiments, hairpin 1 and hairpin 2 are separated by one nucleotide. In some embodiments, the hairpin regions comprise fewer nucleotides than shown in Table 3B. In some embodiments, the hairpin regions comprise more nucleotides than shown in Table 3B. When a hairpin region comprises fewer or more nucleotides than shown in the schematic of Table 3B, the modification pattern, as will be apparent to the skilled artisan, should be maintained.

In some embodiments, a hairpin region has nucleotides that are complementary in nucleic acid sequence when read in opposite directions. In some embodiments, the hairpin regions may not be perfectly complimentary to each other when read in opposite directions (e.g., the top or loop of the hairpin comprises unpaired nucleotides).

3′ Terminus

The sgRNA has a 3′ end, which is the last nucleotide of the sgRNA. The 3′ terminus region includes the last 1-7 nucleotides from the 3′ end. In some embodiments, the 3′ end is the end of hairpin 2. In some embodiments, the sgRNA comprises nucleotides after the hairpin region(s). In some embodiments, the sgRNA includes a 3′ tail region, in which case the last nucleotide of the 3′ tail is the 3′ terminus. In some embodiments, the 3′ tail comprises 1, 2, 3, 4, 5, 6, 7, 8, 9, 10, 15, or 20 or more nucleotides, e.g. that are not associated with the secondary structure of a hairpin. In some embodiments, the 3′ tail region comprises 1, 2, 3, or 4 nucleotides that are not associated with the secondary structure of a hairpin. In some embodiments, the 3′ tail region comprises 4 nucleotides that are not associated with the secondary structure of a hairpin. In some embodiments, the 3′ tail region comprises 1, 2, or 3 nucleotides that are not associated with the secondary structure of a hairpin.

In some embodiments, the spacer or targeting region of the gRNA is present at the 3′ end of the gRNA.

TABLE 3A
(Conserved Portion of a spyCas9 sgRNA; SEQ ID NO: 400)

| 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 | 24 | 25 | 26 | 27 | 28 | 29 | 30 |
|---|---|---|---|---|---|---|---|---|---|---|---|---|---|---|---|---|---|---|---|---|---|---|---|---|---|---|---|---|---|
| G | U | U | U | U | A | G | A | G | C | U | A | G | A | A | A | U | A | G | C | A | A | G | U | U | A | A | A | A | U |
| LS1-LS6 | | | | | | B1-B2 | | US1-US12 | | | | | | | | | | | | B3-B6 | | | | LS7-LS12 | | | | | |
| 31 | 32 | 33 | 34 | 35 | 36 | 37 | 38 | 39 | 40 | 41 | 42 | 43 | 44 | 45 | 46 | 47 | 48 | 49 | 50 | 51 | 52 | 53 | 54 | 55 | 56 | 57 | 58 | 59 | 60 |
| A | A | G | G | C | U | A | G | U | C | C | G | U | U | A | U | C | A | A | C | U | U | G | A | A | A | A | A | G | U |

| Nexus | | | | | | | | | H1-1 through H1-12 | | | | | | |
|---|---|---|---|---|---|---|---|---|---|---|---|---|---|---|---|
| 61 | 62 | 63 | 64 | 65 | 66 | 67 | 68 | 69 | 70 | 71 | 72 | 73 | 74 | 75 | 76 |
| G | G | C | A | C | C | G | A | G | U | C | G | G | U | G | C |

| | |
|---|---|
| N | H2-1 through H2-15 |

TABLE 3B
(Regions of spyCas9 sgRNA (linear view, 5′ to 3′)

| | LS1-6 | | B1-2 | | US1-12 | | B3-6 | |
|---|---|---|---|---|---|---|---|---|
| 5′ | lower | n | bulge | n | upper | n | bulge | n |
| terminus (n) | stem | | | | stem | | | |

| | | | | H1-1 thru | | H2-1 thru | |
|---|---|---|---|---|---|---|---|
| LS7-12 | | N1-18 | | H1-12 | | H2-15 | |
| lower | n | nexus | n | hairpin 1 | n | hairpin 2 | 3′ |
| stem | | | | | | | terminus |

In some embodiments, the sgRNA comprises a conserved portion comprising a sequence of SEQ ID NO: 400.

In some embodiments, 2, 3 or 4 of nucleotides 13-16 (US5-US8 of the upper stem region) are substituted for the first internal linker relative SEQ ID NO: 400. In some embodiments, nucleotides 12-17 (US4-US9 of the upper stem region) are substituted for the first internal linker relative SEQ ID NO: 400. In some embodiments, nucleotides 11-18 (US3-US10 of the upper stem region) are substituted for the first internal linker relative SEQ ID NO: 400. In some embodiments, nucleotides 10-19 (US2-US11 of the upper stem region) are substituted for the first internal linker relative SEQ ID NO: 400. In some embodiments, nucleotides 9-20 (US1-US10 of the upper stem region) are substituted for the first internal linker relative SEQ ID NO: 400. In some embodiments, nucleotide 36-37 (N6-N7 of the nexus region) are substituted for the second internal linker relative SEQ ID NO: 400. In some embodiments, 2, 3, or 4 of nucleotides 53-56 (H1-5-H1-8 of the hairpin 1) are substituted for the third internal linker relative SEQ ID NO: 400. In some embodiments, nucleotides 52-57 (H1-4-H1-9 of the hairpin 1) are substituted for the third internal linker relative SEQ ID NO: 400. In some embodiments, nucleotides 51-58 (H1-3-H1-10 of the hairpin 1) are substituted for the third internal linker relative SEQ ID NO: 400. In some embodiments, nucleotides 50-59 (H1-1-H1-12 of the hairpin 1) are substituted for the third internal linker relative SEQ ID NO: 400. In some embodiments, nucleotides 77-80 are deleted relative SEQ ID NO: 400. In some embodiments, all of the nucleotides of the upper stem (US1-US12) are substituted for the first internal linker relative to SEQ ID NO: 400. In some embodiments, all of the nucleotides of the upper stem (US1-US12) are substituted with an abasic nucleoside relative to SEQ ID NO: 400 in a sgRNA wherein nucleotides in another portion of the sgRNA is substituted for an internal linker, e.g., in the nexus region or preferably in the hairpin 1 region as provided above.

G. NmeCas9 Guide RNAs with One or More Shortened Regions Comprising Internal Linker(s)

Provided herein are guide RNAs (gRNAs) comprising one or more shortened regions and one or more internal linker.

In some embodiments, a gRNA (e.g., sgRNA, dgRNA, or crRNA) provided herein comprises a conserved region comprising a repeat/anti-repeat region, a hairpin 1 region, and a hairpin 2 region, wherein one or more of the repeat/anti-repeat region, the hairpin 1 region, and the hairpin 2 region are shortened. In some embodiments, the gRNA is an N. meningitidis Cas9 (NmeCas9) gRNA.

In some embodiments, the conserved region of a gRNA comprises:

a shortened repeat/anti-repeat region, wherein the shortened repeat/anti-repeat region lacks 2-24 nucleotides, wherein
(i) one or more of nucleotides 37-64 is deleted and optionally substituted relative to SEQ ID NO: 500; and
(ii) nucleotide 36 is linked to nucleotide 65 by (i) a first internal linker that alone or in combination with nucleotides substitutes for 4 nucleotides, or (ii) at least 4 nucleotides.

In some embodiments, the conserved region of a gRNA comprises:
a shortened hairpin 1 region, wherein the shortened hairpin 1 lacks 2-10, optionally 2-8 nucleotides, wherein
(i) one or more of nucleotides 82-95 is deleted and optionally substituted relative to SEQ ID NO: 500; and
(ii) nucleotide 81 is linked to nucleotide 96 by (i) a second internal linker that alone or in combination with nucleotides substitutes for 4 nucleotides, or (ii) at least 4 nucleotides.

In some embodiments, the conserved region of a gRNA comprises:
a shortened hairpin 2 region, wherein the shortened hairpin 2 lacks 2-18, optionally 2-16 nucleotides, wherein
(i) one or more of nucleotides 113-134 is deleted and optionally substituted relative to SEQ ID NO: 500; and
(ii) nucleotide 112 is linked to nucleotide 135 by (i) a third internal linker that alone or in combination with nucleotides substitutes for 4 nucleotides, or (ii) at least 4 nucleotides.

In some embodiments, the conserved region of a gRNA comprises:
(a) a shortened repeat/anti-repeat region, wherein the shortened repeat/anti-repeat region lacks 2-24 nucleotides, wherein
   (i) one or more of nucleotides 37-64 is deleted and optionally substituted relative to SEQ ID NO: 500; and
   (ii) nucleotide 36 is linked to nucleotide 65 by (i) a first internal linker that alone or in combination with nucleotides substitutes for 4 nucleotides, or (ii) at least 4 nucleotides; and
   (b) a shortened hairpin 1 region, wherein the shortened hairpin 1 lacks 2-10, optionally 2-8 nucleotides, wherein
   (i) one or more of nucleotides 82-95 is deleted and optionally substituted relative to SEQ ID NO: 500; and
   (ii) nucleotide 81 is linked to nucleotide 96 by (i) a second internal linker that alone or in combination with nucleotides substitutes for 4 nucleotides, or (ii) at least 4 nucleotides.

In some embodiments, the conserved region of a gRNA comprises:
(a) a shortened repeat/anti-repeat region, wherein the shortened repeat/anti-repeat region lacks 2-24 nucleotides, wherein
   (i) one or more of nucleotides 37-64 is deleted and optionally substituted relative to SEQ ID NO: 500; and
   (ii) nucleotide 36 is linked to nucleotide 65 by (i) a first internal linker that alone or in combination with nucleotides substitutes for 4 nucleotides, or (ii) at least 4 nucleotides; and
   (b) a shortened hairpin 2 region, wherein the shortened hairpin 2 lacks 2-18, optionally 2-16 nucleotides, wherein
   (i) one or more of nucleotides 113-134 is deleted and optionally substituted relative to SEQ ID NO: 500; and
   (ii) nucleotide 112 is linked to nucleotide 135 b by (i) a third internal linker that alone or in combination with nucleotides substitutes for 4 nucleotides, or (ii) at least 4 nucleotides.

In some embodiments, the conserved region of a gRNA comprises:
(a) a shortened hairpin 1 region, wherein the shortened hairpin 1 lacks 2-10, optionally 2-8 nucleotides, wherein
   (i) one or more of nucleotides 82-95 is deleted and optionally substituted relative to SEQ ID NO: 500; and
   (ii) nucleotide 81 is linked to nucleotide 96 by (i) a second internal linker that alone or in combination with nucleotides substitutes for 4 nucleotides, or (ii) at least 4 nucleotides; and
   (b) a shortened hairpin 2 region, wherein the shortened hairpin 2 lacks 2-18, optionally 2-16 nucleotides, wherein
   (i) one or more of nucleotides 113-134 is deleted and optionally substituted relative to SEQ ID NO: 500; and
   (ii) nucleotide 112 is linked to nucleotide 135 b by (i) a third internal linker that alone or in combination with nucleotides substitutes for 4 nucleotides, or (ii) at least 4 nucleotides.

In some embodiments, the conserved region of a gRNA comprises:
(a) a shortened repeat/anti-repeat region, wherein the shortened repeat/anti-repeat region lacks 2-24 nucleotides, wherein
   (i) one or more of nucleotides 37-64 is deleted and optionally substituted relative to SEQ ID NO: 500; and
   (ii) nucleotide 36 is linked to nucleotide 65 by (i) a first internal linker that alone or in combination with nucleotides substitutes for 4 nucleotides, or (ii) at least 4 nucleotides;
   (b) a shortened hairpin 1 region, wherein the shortened hairpin 1 lacks 2-10, optionally 2-8, nucleotides, wherein
   (i) one or more of nucleotides 82-95 is deleted and optionally substituted relative to SEQ ID NO: 500; and
   (ii) nucleotide 81 is linked to nucleotide 96 by (i) a second internal linker that alone or in combination with nucleotides substitutes for 4 nucleotides, or (ii) at least 4 nucleotides; and
   (c) a shortened hairpin 2 region, wherein the shortened hairpin 2 lacks 2-18, optionally 2-16, nucleotides, wherein
   (i) one or more of nucleotides 113-134 is deleted and optionally substituted relative to SEQ ID NO: 500; and
   (ii) nucleotide 112 is linked to nucleotide 135 by (i) a third internal linker that alone or in combination with nucleotides substitutes for 4 nucleotides, or (ii) at least 4 nucleotides.

Nucleotide positions in this section, including subsections A-E below, are numbered according to FIG. 10E which provides an exemplary Nine sgRNA.

In some embodiments, one or both nucleotides 144-145 are optionally deleted as compared to SEQ ID NO: 500.

In some embodiments, the gRNA comprises at least one of the first internal linker, the second internal linker, and the third internal linker.

In some embodiments, the gRNA comprises at least two of the first internal linker, the second internal linker, and the third internal linker.

In some embodiments, the gRNA comprises the first internal linker, the second internal linker, and the third internal linker.

In some embodiments, the guide region has (i) an insertion of one nucleotide or a deletion of 1-4 nucleotides within positions 1-24 relative to SEQ ID NO: 500, or (ii) a length of 24 nucleotides.

In some embodiments, the guide region has a length of 25, 24, 23, 22, 21, or 20 nucleotides, optionally wherein the guide region has a length of 25, 24, 23, or 22 nucleotides at positions 1-24 of SEQ ID NO: 500.

In some embodiments, the guide region has a length of 23 or 24 nucleotides at positions 1-24 of SEQ ID NO: 500.

In some embodiments, at least 10 nucleotides of the conserved portion are modified nucleotides.

In some embodiments, a substitution in a duplex portion is a conservative substitution.

Within each the repeat/anti-repeat region, the hairpin 1 region, and the hairpin 2 region, the strands of each of the duplex portions are joined by an internal linker that alone or in combination with nucleotides substitutes for 4 nucleotides, or at least 4 nucleotides. Provided herein are internal linkers having various bridging lengths to permit one of skill in the art to join the strands of the duplex portion with internal linkers or nucleotides or a combination thereof.

In some embodiments, a repeat/anti-repeat region of a gRNA is a shortened repeat/anti-repeat region lacking 2-24 nucleotides, e.g., any of the repeat/anti-repeat regions indicated in the numbered embodiments above or Tables 1-2 or described elsewhere herein, which may be combined with any of the shortened hairpin 1 region or hairpin 2 region described herein, including but not limited to combinations indicated in the numbered embodiments above and represented in the sequences of Tables 1-2 or described elsewhere herein.

In some embodiments, the first linker substitutes positions 49-52 and the second internal linker substitutes positions 87-90.

In some embodiments, the second internal linker substitutes positions 87-90 and the third internal linker substitutes positions 122-125.

In some embodiments, the first linker substitutes positions 49-52, and the third internal linker substitutes positions 122-125.

In some embodiments, the first linker substitutes 49-52, the second internal linker substitutes positions 87-90, and the third internal linker substitutes positions 122-125.

Shortened Repeat/Anti-Repeat Region

In some embodiments, a conserved portion of a gRNA described herein comprises a shortened repeat/anti-repeat region. In some embodiments, the repeat-anti-repeat region comprises a hairpin structure between a first portion and a second portion of the repeat-anti-repeat region, wherein the first portion and the second portion together form a duplex portion.

In some embodiments, a conserved portion of a gRNA described herein comprises a shortened upper stem region of the repeat/anti-repeat region. In some embodiments, the repeat/anti-repeat region comprises a loop (e.g., a tetraloop).

In some embodiments, the shortened repeat/anti-repeat region lacks 2-28 nucleotides. In some embodiments, (i) one or more of nucleotides 37-64 is deleted and optionally substituted relative to SEQ ID NO: 1; and (ii) nucleotide 36 is linked to nucleotide 65 by a first internal linker.

In some embodiments, the shortened repeat/anti-repeat region lacks 2-28 nucleotides.

In some embodiments, the shortened repeat/anti-repeat region has a length of 24, 25, 26 27, 28, 29, 30, 31, 32, 33, 34, 35, 36, 37, 38, 39, 40, 41, 42, 43, 44, 45, 46, 47, 48, 49, or 50 nucleotides.

In some embodiments, the shortened repeat/anti-repeat region lacks 12-28, optionally 18-24 nucleotides. In some embodiments, the shortened repeat/anti-repeat region has a length of 24, 25, 26, 27, 28, 29, 30, 31, 32, 33, 34, 35, 36, 37, 38, 39, or 40 nucleotides. In some embodiments, the shortened repeat/anti-repeat region has a length of 24, 25, 26, 27, 28, 29, 30, 31, 32, 33, or 34 nucleotides. In some embodiments, the shortened repeat/anti-repeat region has a length of 34 nucleotides. In some embodiments, the shortened repeat/anti-repeat region has a length of 35 nucleotides. In some embodiments, the shortened repeat/anti-repeat region has a length of 36 nucleotides. In some embodiments, the shortened repeat/anti-repeat region has a length of 37 nucleotides. In some embodiments, the shortened repeat/anti-repeat region has a length of 38 nucleotides.

In some embodiments, one or more base pairs of the upper stem of the shortened repeat/anti-repeat region are deleted. In some embodiments, the upper stem of the shortened repeat/anti-repeat region comprises no more than one, two, three, or four base pairs. As used herein, “base pairs” or “base paired nucleotides” or “Watson-Crick pairing nucleotides” include any pair capable of forming a Watson-Crick base pair, including A-T, A-U, T-A, U-A, C-G, and G-C pairs, and pairs including modified versions of any of the foregoing nucleotides that have the same base pairing preference. In some embodiments, base pairs or base paired nucleotides also include base pairs generated by base stacking, e.g. nucleotides 25 and 76, 33 and 68, 34 and 67, and 37 and 64 in the repeat/anti-repeat region; and nucleotides 78 and 100, and 83 and 94 in the hairpin 1 region.

In some embodiments, the first internal linker substitutes nucleotides 38-63 of the upper stem of the shortened repeat/anti-repeat region and links nucleotide 37 to nucleotide 64. In some embodiments, the first internal linker substitutes nucleotides 37-64 of the upper stem of the shortened repeat/anti-repeat region and links nucleotide 36 to nucleotide 65.

In some embodiments, the shortened repeat/anti-repeat region has a duplex portion 11 base paired nucleotides in length. In some embodiments, the shortened repeat/anti-repeat region has a single duplex portion. In some embodiments, positions 25 and 76, positions 33 nad 68, positions 34 and 67, and positions 48 and 53 have base stacking interactions and do not constitute a discontinuity in the duplex portion.

In some embodiments, one or more of base paired nucleotides in the repeat/anti-repeat region is deleted. In some embodiments, one or more of based paired nucleotides chosen from positions 37 and 53, positions 38 and 54, position 39 and 55, positions 40 and 56, positions 41 and 57, positions 43 and 58, positions 43 and 59, positions 44 and 60, positions 45 and 61, positions 46 and 62, positions 47 and 63, and positions 48 and 64.

In some embodiments, the upper stem region of the repeat/anti-repeat region comprises 1-5 base pairs.

In some embodiments, the upper stem of the shortened repeat/anti-repeat region includes one or more substitution relative to SEQ ID NO: 500.

In some embodiments, one or more substitutions are considered conservative substitutions by exchanging positions of base paired nucleotides such that base pairings may be maintained. For example, a G-C pair becomes a C-G pair, an A-U pair becomes a U-A pair, or other natural or modified base pairing.

In some embodiments, the first internal linker substitutes nucleotides 49-52 is substituted relative to SEQ ID NO: 500.

In some embodiments, the shortened repeat/anti-repeat region has 8-22 modified nucleotides.

Shortened Hairpin 1 Region

In some embodiments, a conserved portion of a gRNA described herein comprises a shortened hairpin 1 region. In some embodiments, the hairpin 1 region comprises a hairpin structure between a first portion and a second portion of the hairpin 1 region, wherein the first portion and the second portion together form a duplex portion.

In some embodiments, a conserved portion of a gRNA described herein comprises a shortened upper stem region of the hairpin 1 region. In some embodiments, the hairpin 1 comprises a loop (e.g., a tetraloop).

In some embodiments, the shortened hairpin 1 lacks 2-10 nucleotides. In some embodiments, (i) one or more of nucleotides 82-95 is deleted and optionally substituted relative to SEQ ID NO: 500; and (ii) nucleotide 81 is linked to nucleotide 96 by a second internal linker.

In some embodiments, wherein the shortened hairpin 1 region lacks 2-10 nucleotides. In some embodiments, wherein the shortened hairpin 1 region has a length of 13, 14, 15, 16, 17, 18, 19, 20 or 21 nucleotides. In some embodiments, wherein the shortened hairpin 1 region has duplex portion 4-8 base paired nucleotides in length. In some embodiments, wherein the shortened hairpin 1 region has duplex portion 7-8 base paired nucleotides in length.

In some embodiments, wherein the shortened hairpin 1 region has a single duplex portion. In some embodiments, in the shortened hairpin 1 region, positions 78 and 100, and positions 83 and 94 have base stacking interactions and do not constitute a discontinuity in the duplex portion.

In some embodiments, one or two base pairs of the shortened hairpin 1 region are deleted. In some embodiments, the stem of the shortened hairpin 1 region comprises one, two, three, four, five, six, seven or eight base pairs. In some embodiments, the stem of the shortened hairpin 1 region is seven or eight base paired nucleotides in length.

In some embodiments, one or more of positions 85-86 and one or more of nucleotides 91-92 of the shortened hairpin 1 region are deleted. In some embodiments, nucleotides 86 and 91 of the shortened hairpin 1 region are deleted. In some embodiments, one or more of nucleotides 82-95 of the shortened hairpin 1 region is substituted relative to SEQ ID NO: 500.

In some embodiments, the second internal linker substitutes nucleotides 87-91 relative to SEQ ID NO: 500.

In some embodiments, wherein the shortened hairpin 1 region has 2-15 modified nucleotides.

Shortened Hairpin 2 Region

In some embodiments, a conserved portion of a gRNA described herein comprises a shortened hairpin 2 region. In some embodiments, the shortened hairpin 2 region comprises a hairpin structure between a first portion and a second portion of the hairpin 2 region, wherein the first portion and the second portion together form a duplex portion.

In some embodiments, the shortened hairpin 2 region lacks 2-18 nucleotides. In some embodiments, the shortened hairpin 2 region lacks 2-16 nucleotides. In some embodiments, (i) one or more of nucleotides 113-121 and 126-134 is deleted and optionally substituted relative to SEQ ID NO: 500; and (ii) nucleotide 112 is linked to nucleotide 135 by a third internal linker.

In some embodiments, a conserved portion of a gRNA described herein comprises a shortened upper stem region of the hairpin 2 region. In some embodiments, the hairpin 1 comprises a loop (e.g., a tetraloop). In some embodiments, the shortened hairpin 2 region lacks 2-16 nucleotides. In some embodiments, the shortened hairpin 2 region has a length of 22, 23, 24, 25, 26, 27, 28, 29, 30, 31, 32, 33, 34, 35, 36, 37, 38, 39, or 40 nucleotides. In some embodiments, the shortened hairpin 2 region has a length of 24, 25, 26, 27, 28, 29, 30, 31, 32, 33 or 34, nucleotides. In some embodiments, one or more of nucleotides 113-121 and one or more of nucleotides 126-134 of the shortened hairpin 2 region are deleted.

In some embodiments, the shortened hairpin 2 region comprises an unpaired region.

In some embodiments, the shortened hairpin 2 region has two duplex portions. In some embodiments, the shortened hairpin 2 region has a duplex portion of 4 base paired nucleotides in length. In some embodiments, the shortened hairpin 2 region has a duplex portion of 4-8 base paired nucleotides in length. In some embodiments, the shortened hairpin 2 region has a duplex portion of 4-6 base paired nucleotides in length. In some embodiments, the upper stem of the shortened hairpin 2 region comprises one, two, three, or four base pairs. In some embodiments, at least one pair of nucleotides 113 and 134, nucleotides 114 and 133, nucleotides 115 and 132, nucleotides 116 and 131, nucleotides 117 and 130, nucleotides 118 and 129, nucleotides 119 and 128, nucleotides 120 and 127, and nucleotides 121 and 126 are deleted. In some embodiments, all of positions 113-121 and 126-134 of the shortened hairpin 2 region are deleted.

In some embodiments wherein one or more of nucleotides 113-134 of the shortened hairpin 2 region is substituted relative to SEQ ID NO: 500. In some embodiments, the third internal linker substitutes nucleotides 122-125 relative to SEQ ID NO: 500.

In some embodiments the shortened hairpin 2 region has 2-15 modified nucleotides.

3′ Tail

In some embodiments, the gRNA comprises a 3′ tail. In some embodiments, the 3′ tail is 1-20 nucleotides in length and is linked by a phosphodiester or a phosphorothioate linkage, to the 3′ end of the conserved region of a gRNA. In some embodiments, the 3′ tail comprises 1, 2, 3, 4, 5, 6, 7, 8, 9, or 10 nucleotides. In some embodiments, the 3′ tail comprises 1, 2, 3, 4, or 5 nucleotides. In some embodiments, the 3′ tail comprises 1 or 2 nucleotides.

In some embodiments, the 3′ tail has a length of 1-10 nucleotides, 1-5 nucleotides, 1-4 nucleotides, 1-3 nucleotides, and 1-2 nucleotides. In some embodiments, the 3′ tail comprises about 1, 2, 3, 4, 5, 6, 7, 8, 9, or 10 nucleotides. In some embodiments, the 3′ tail has a length of 1 nucleotide. In some embodiments, the 3′ tail has a length of 2 nucleotides. In some embodiments, the 3′ tail has a length of 3 nucleotides. In some embodiments, the 3′ tail has a length of 4 nucleotides. In some embodiments, the 3′ tail has a length of 1-2, nucleotides.

In some embodiments, the 3′ tail terminates with a nucleotide comprising a uracil or modified uracil. In some embodiments, the 3′ tail is 1 nucleotide in length. In some embodiments, the 3′ tail consists of a nucleotide comprising a uracil or a modified uracil. In some embodiments, wherein the 3′ tail comprises a modification of any one or more of the nucleotides present in the 3′ tail. In further embodiments, wherein the modification of the 3′ tail is one or more of 2′-O-methyl (2′-OMe) modified nucleotide and a phosphorothioate (PS) linkage between nucleotides.

In some embodiments, wherein the 3′ tail is fully modified.

In some embodiments, wherein the 3′ nucleotide of the gRNA is a nucleotide comprising a uracil or a modified uracil.

In some embodiments, one or more of nucleotides 144 and 145 are deleted relative to SEQ ID NO: 500. In some embodiments, both nucleotides 144 and 145 are deleted relative to SEQ ID NO: 500.

In some embodiments, the gRNA does not comprise a 3′ tail.

In some embodiments, the 3′ end of the guide, that does not comprise a 3′ tail, terminates with a nucleotide comprising a uracil or modified uracil. In some embodiments, the 3′ tail consists of a nucleotide comprising a uracil or modified uracil. In some embodiments, the 3′ terminal nucleotide is a modified nucleotide. In some embodiments, the modification of the 3′ end is one or more of 2′-O-methyl (2′-OMe) modified nucleotide and a phosphorothioate (PS) linkage between nucleotide the terminal nucleotide and the penultimate nucleotide.

In some embodiments, the 3′ end, i.e., the end of hairpin 2 with no further tail or the end of the 3′ tail, comprises or further comprises one or more modifications, e.g., a phosphorothioate (PS) linkage between nucleotides, a 2′-OMe modified nucleotide, a 2′-O-moe modified nucleotide, a 2′-F modified nucleotide, or an inverted abasic modified nucleotide, optionally wherein the 3′ end comprises at least two modifications independently selected from a phosphorothioate (PS) linkage between nucleotides, a 2′-OMe modified nucleotide, a 2′-O-moe modified nucleotide, a 2′-F modified nucleotide, and an inverted abasic modified nucleotide. In some embodiments, the 3′ end comprises or further comprises one or more modifications, e.g., a phosphorothioate (PS) linkage between nucleotides, a 2′-OMe modified nucleotide, a 2′-F modified nucleotide, optionally wherein the 3′ end comprises at least two modifications independently selected from a phosphorothioate (PS) linkage between nucleotides, a 2′-OMe modified nucleotide, and a 2′-F modified nucleotide. In some embodiments, the 3′ end comprises phosphorothioate (PS) linkage between nucleotides 141 and 142, and 142 and 143; a 2′-OMe modified nucleotide at each of positions 142 and 143.

In some embodiments, the 3′ end, i.e., the end of hairpin 2 with no further tail or the end of the 3′ tail, comprises or further comprises one or more phosphorothioate (PS) linkages between nucleotides. In some embodiments, the 3′ end comprises or further comprises one or more 2′-OMe modified nucleotides. In some embodiments, the 3′ end comprises or further comprises one or more 2′-O-moe modified nucleotides. In some embodiments, the 3′ end comprises or further comprises one or more 2′-F modified nucleotide. In some embodiments, the 3′ end comprises or further comprises one or more an inverted abasic modified nucleotides. In some embodiments, the 3′ end comprises or further comprises one or more protective end modifications. In some embodiments, the 3′ end comprises or further comprises a combination of one or more of a phosphorothioate (PS) linkage between nucleotides, a 2′-OMe modified nucleotide, a 2′-O-moe modified nucleotide, a 2′-F modified nucleotide, and an inverted abasic modified nucleotide.

Guide Region

In some embodiments, the gRNA further comprises a guide sequence. In some embodiments, the guide sequence comprises 20, 21, 22, 23, 24, or 25 nucleotides, optionally 22, 23, 24, or 25 nucleotides 5′ to the most 5′ nucleotide of the repeat/anti-repeat region. In some embodiments, the guide sequence comprises 22, 23, 24, 25, or more nucleotides. In some embodiments, the guide sequence has a has a length of 24 nucleotides. In some embodiments, the guide sequence has a length of 23 nucleotides. In some embodiments, the guide sequence has a length of 22 nucleotides. In some embodiments, the guide sequence has a length of 21 nucleotides. In some embodiments, the guide sequence has a length of 20 nucleotides.

In some embodiments, the guide region has (i) an insertion of one nucleotide or a deletion of 1-4 nucleotides within positions 1-24 relative to SEQ ID NO: 500, or (ii) a length of 24 nucleotides.

In some embodiments, the selection of the guide sequence is determined based on target sequences within the gene of interest for editing. For example, in some embodiments, the gRNA comprises a guide sequence that is complementary to target sequences of a gene of interest.

In some embodiments, the target sequence in the gene of interest may be complementary to the guide sequence of the gRNA. In some embodiments, the degree of complementarity or identity between a guide sequence of a gRNA and its corresponding target sequence in the gene of interest may be about 90%, 95%, or 100%. In some embodiments, the guide region of a gRNA and the target region of a gene of interest may be 100% complementary or identical. In other embodiments, the guide sequence of a gRNA and the target sequence of a gene of interest may contain at least one mismatch. For example, the guide sequence of a gRNA and the target sequence of a gene of interest may contain 1, optionally 2, or 3mismatches, where the total length of the target sequence is at least about 22, 23, 24, or more nucleotides. In some embodiments, the guide sequence of a gRNA and the target region of a gene of interest may contain 1, optionally 2, or 3 mismatches where the guide sequence comprises about 24 nucleotides. In certain embodiments, the guide sequence contains no mismatches, i.e., is fully complementary, to the target sequence. The 5′ terminus may comprise nucleotides that are not considered guide regions (i.e., do not function to direct a Cas9 protein to a target nucleic acid).

In some embodiments, the guide region of the shortened guide RNA comprises at least one modified nucleotide.
In some embodiments, the guide region of the gRNA comprises at least two modified nucleotides, optionally at least four modified nucleotide, wherein each modification, independently, optionally comprises a modified nucleotide selected from 2′-O-methyl (2′-OMe) modified nucleotide, 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide, or combinations thereof.

Exemplary shortened guide RNAs comprising internal linkers are provided in Tables 4A-4B. As used herein, “Linker 1” or “L1” refers to an internal linker having a bridging length of about 15-21 atoms. As used herein, “Linker 2” or “L2” refers to an internal linker having a bridging length of about 6-12 atoms.

Nucleotide modifications are indicated in Tables 4A-4B as follows: m: 2′-OMe; *: PS linkage; f 2′-fluoro; (invd): inverted abasic; moe: 2′-moe; e: ENA; d: deoxyribonucleotide (also note that T is always a deoxyribonucleotide); x: UNA. In the sgRNA modified sequences, in certain embodiments, each A, C, G, U, and N is independently a ribose sugar (2′-OH). In certain embodiments, each A, C, G, U, and N is a ribose sugar (2′-OH). Thus, for example, mA represents 2′-O-methyl adenosine; xA represents a UNA nucleotide with an adenine nucleobase; eA represents an ENA nucleotide with an adenine nucleobase; and dA represents an adenosine deoxyribonucleotide.

As used herein, “N” may be any natural or non-natural nucleotide. For example, encompassed herein is SEQ ID NO: 1001 in Table 4A, where the N's are replaced with any of the guide sequences disclosed herein. The modifications remain as shown in SEQ ID NO: 1001 despite the substitution of N's for the nucleotides of a guide. That is, although the nucleotides of the guide replace the “N's”, the first three nucleotides are 2′-O-Me modified and there are phosphorothioate linkages between the first and second nucleotides, the second and third nucleotides and the third and fourth nucleotides.

sgRNA designations are sometimes provided with one or more leading zeroes immediately following the G. This does not affect the meaning of the designation. Thus, for example, G000282, G0282, G00282, and G282 refer to the same sgRNA.

TABLE 4A
Exemplary NmeCas9 guide RNAs comprising linkers

| SEQ | |
|---|---|
| ID | |
| NO: | gRNA sequence |
| 1000 | (N)₂₀₋₂₅ |
| | GUUGUAGCUCCCUUC(L1)GACCGUUGCUACAAUAAGG |
| | CCGUC(L1)GAUGUGCCGCAACGCUCUGCC(L1)GGCA |
| | UCGUU |
| 1001 | mN*mN*mN*mNmNNNmNmNNmNNmNNNNNmNNNNmNNN |
| | mGUUGmUmAmGmCUCCCmUmUmC(L1)mGmAmCmCGUU |
| | mGmCUAmCAAU*AAGmGmCCmGmUmC(L1)mGmAmUGU |
| | GCmCGmCAAmCGCUCUmGmCC(L1)GGCAUCG*mU*mU |
| 1002 | mN*mN*mN*mNmNNNmNmNNmNNmNNNNNmNNNNmNNN |
| | mGUUGmUmAmGmCUCCCmU(L1)mCmCGUUmGmCUAmC |
| | AAU*AAGmGmCCmGmUmC(L1)mGmAmUGUGCmCGmCA |
| | AmCGCUCUmGmCC(L1)GGCAUCG*mU*mU |
| 1003 | NNNNNNNNNNNNNNNNNNNNNNNNmGUUGmUmAmGmCU |
| | CCCmU(L1)mCmCGUUmGmCUAmCAAU*AAGmGmCCmG |
| | mUmC(L1)mGmAmUGUGCmCGmCAAmCGCUCUmGmCC |
| | (L1)GGCAUCG*mU*mU |
| 1004 | (N)₂₀₋₂₅ |
| | GUUGmUmAmGmCUCCCmU(L1)mCmCGUUmGmCUAmCA |
| | AU*AAGmGmCCmGmUmC(L1)mGmAmUGUGCmCGmCAA |
| | mCGCUCUmGmCC(L1)GGCAUCG*mU*mU |
| 1005 | (N)₂₀₋₂₅ |
| | GUUGmUmAmGmCUCCCmUmUmG(L1)mCmAmCmCGUUm |
| | GmCUAmCAAU*AAGmGmCCmGmUmC(L1)mGmAmUGUG |
| | CmCGmCAAmCGCUCUmGmCC(L1)GGCAUCGmU*mU |
| 1006 | (N)₂₀₋₂₅ |
| | GUUGmUmAmGmCUCCCmUmUmG(L1)mCmAmCmCGUUm |
| | GmCUAmCAAU*AAGmGmCCmGmUmC(L1)mGmAmUGUG |
| | CmCGmCAAmCGCUCUmGmCC(L1)GGCAUCGmU*mU |
| 1007 | (N)₂₀₋₂₅ |
| | GUUGmUmAmGmCUCCCmUmG(L1)mCmCmCGUUmGmCU |
| | AmCAAU*AAGmGmCCmGmUmC(L1)mGmAmUGUGCmCG |
| | mCAAmCGCUCUmGmCC(L1)GGCAUCGmU*mU |
| 1008 | (N)₂₀₋₂₅ |
| | GUUGmUmAmGmCUCCCmG(L1)mCmCGUUmGmCUAmCA |
| | AU*AAGmGmCCmGmUmC(L1)mGmAmUGUGCmCGmCAA |
| | mCGCUCUmGmCC(L1)GGCAUCGmU*mU |
| 1009 | (N)₂₀₋₂₅ |
| | GUUGmUmAmGmCUCCmG(L1)mCGUUmGmCUAmCAAU* |
| | AAGmGmCCmGmUmC(L1)mGmAmUGUGCmCGmCAAmCG |
| | CUCUmGmCC(L1)GGCAUCGmU*mU |
| 1010 | (N)₂₀₋₂₅ |
| | GUUGmUmAmGmCUCC(L1)GUUmGmCUAmCAAU*AAGm |
| | GmCCmGmUmC(L1)mGmAmUGUGCmCGmCAAmCGCUCU |
| | mGmCC(L1)GGCAUCGmU*mU |
| 1011 | (N)₂₀₋₂₅ |
| | GUUGmUmAmGmCUCCCmUmUmG(L1)mCmAmCmCGUUm |
| | GmCUAmCAAU*AAGmGmCCmGmUmCmU(L1)mAmGmAm |
| | UGUGCmCGmCAAmCGCUCUmGmCC(L1)GGCAUCGmU* |
| | mU |
| 1012 | (N)₂₀₋₂₅ |
| | GUUGmUmAmGmCUCCCmUmUmG(L1)mCmAmCmCGUUm |
| | GmCUAmCAAU*AAGmGmCCmGmUmC(L1)mGmAmUGUG |
| | CmCGmCAAmCGCUCUmGmCCmU(L1)mAGGCAUCGmU* |
| | mU |

TABLE 4B
Exemplary NmeCas9 gRNA Sequences

| | SEQ | | SEQ | |
|---|---|---|---|---|
| Guide | ID | sgRNA unmodified | ID | |
| ID | NO. | sequence | NO. | sgRNA modified sequence |
| G021536 | 610 | CCAAGUGUCUUCCAG | 710 | mC*mC*mA*mAmGUGmUmCUmUCm |
| | | UACGAUUUGGUUGUA | | CAGUAmCGAUmUUGmGUUGmUmA |
| | | GCUCCCUGAAACCGU | | mGmCUCCCmUmGmAmAmAmCmCG |
| | | UGCUACAAUAAGGCC | | UUmGmCUAmCAAU*AAGmGmCCm |
| | | GUCGAAAGAUGUGCC | | GmUmCmGmAmAmAmGmAmUGUGC |
| | | GCAACGCUCUGCCUU | | mCGmCAAmCGCUCUmGmCCmUmU |
| | | CUGGCAUCGUU | | mCmUGGCAUCG*mU*mU |
| G021844 | 611 | CCAAGUGUCUUCCAG | 711 | mC*mC*mA*mAmGUGmUmCUmUCm |
| | | UACGAUUUGGUUGUA | | CAGUAmCGAUmUUGmGUUGmUmA |
| | | GCUCCCUUCGACCGU | | mGmCUCCCmUmUmC(L1)mGmAmCm |
| | | UGCUACAAUAAGGCC | | CGUUmGmCUAmCAAU*AAGmGmCC |
| | | GUCGAUGUGCCGCAA | | mGmUmC(L1)mGmAmUGUGCmCGmC |
| | | CGCUCUGCCGGCAUC | | AAmCGCUCUmGmCC(L1)GGCAUCG |
| | | GUU | | *mU*mU |
| G021844 | 612 | CCAAGUGUCUUCCAG | 712 | mC*mC*mA*mAmGUGmUmCUmUCm |
| | | UACGAUUUGGUUGUA | | CAGUAmCGAUmUUGmGUUGmUmA |
| | | GCUCCCUUCGACCGU | | mGmCUCCCmUmUmC(L1)mGmAmCm |
| | | UGCUACAAUAAGGCC | | CGUUmGmCUAmCAAU*AAGmGmCC |
| | | GUCGAUGUGCCGCAA | | mGmUmC(L1)mGmAmUGUGCmCGmC |
| | | CGCUCUGCCGGCAUC | | AAmCGCUCUmGmCC(L1)GGCAUCG |
| | | GUU | | *mU*mU |
| G021845 | 613 | CUUCACCAGGAGAAG | 713 | mC*mU*mU*mCmACCmAmGGmAGm |
| | | CCGUCACACGUUGUA | | AAGCCmGUCAmCACmGUUGmUmA |
| | | GCUCCCUGAAACCGU | | mGmCUCCCmUmGmAmAmAmCmCG |
| | | UGCUACAAUAAGGCC | | UUmGmCUAmCAAU*AAGmGmCCm |
| | | GUCGAAAGAUGUGCC | | GmUmCmGmAmAmAmGmAmUGUGC |
| | | GCAACGCUCUGCCUU | | mCGmCAAmCGCUCUmGmCCmUmU |
| | | CUGGCAUCGUU | | mCmUGGCAUCG*mU*mU |
| G021846 | 614 | CUUCACCAGGAGAAG | 714 | mC*mU*mU*mCmACCmAmGGmAGm |
| | | CCGUCACACGUUGUA | | AAGCCmGUCAmCACmGUUGmUmA |
| | | GCUCCCUUCGACCGU | | mGmCUCCCmUmUmC(L1)mGmAmCm |
| | | UGCUACAAUAAGGCC | | CGUUmGmCUAmCAAU*AAGmGmCC |
| | | GUCGAUGUGCCGCAA | | mGmUmC(L1)mGmAmUGUGCmCGmC |
| | | CGCUCUGCCGGCAUC | | AAmCGCUCUmGmCC(L1)GGCAUCG |
| | | GUU | | *mU*mU |
| G023066 | 615 | CCAAGUGUCUUCCAG | 715 | mC*mC*mA*mAmGUGmUmCUmUCm |
| | | UACGAUUUGGUUGUA | | CAGUAmCGAUmUUGmGUUGmUmA |
| | | GCUCCCUCCGUUGCU | | mGmCUCCCmU(L1)mCmCGUUmGmC |
| | | ACAAUAAGGCCGUCG | | UAmCAAU*AAGmGmCCmGmUmC |
| | | AUGUGCCGCAACGCU | | (L1)mGmAmUGUGCmCGmCAAmCGC |
| | | CUGCCGGCAUCGUU | | UCUmGmCC(L1)GGCAUCG*mU*mU |
| G023067 | 616 | CUUCACCAGGAGAAG | 716 | mC*mU*mU*mCmACCmAmGGmAGm |
| | | CCGUCACACGUUGUA | | AAGCCmGUCAmCACmGUUGmUmA |
| | | GCUCCCUCCGUUGCU | | mGmCUCCCmU(L1)mCmCGUUmGmC |
| | | ACAAUAAGGCCGUCG | | UAmCAAU*AAGmGmCCmGmUmC |
| | | AUGUGCCGCAACGCU | | (L1)mGmAmUGUGCmCGmCAAmCGC |
| | | CUGCCGGCAUCGUU | | UCUmGmCC(L1)GGCAUCG*mU*mU |
| G023069 | 617 | CUUCACCAGGAGAAG | 717 | CUUCACCAGGAGAAGCCGUCACAC |
| | | CCGUCACACGUUGUA | | mGUUGmUmAmGmCUCCCmU(L1)mC |
| | | GCUCCCUCCGUUGCU | | mCGUUmGmCUAmCAAU*AAGmGm |
| | | ACAAUAAGGCCGUCG | | CCmGmUmC(L1)mGmAmUGUGCmCG |
| | | AUGUGCCGCAACGCU | | mCAAmCGCUCUmGmCC(L1)GGCAU |
| | | CUGCCGGCAUCGUU | | CG*mU*mU |
| G023070 | 618 | CUUCACCAGGAGAAG | 718 | mC*mU*mU*CACCAGGAGAAGCCG |
| | | CCGUCACACGUUGUA | | UCACACmGUUGmUmAmGmCUCCC |
| | | GCUCCCUCCGUUGCU | | mU(L1)mCmCGUUmGmCUAmCAAU* |
| | | ACAAUAAGGCCGUCG | | AAGmGmCCmGmUmC(L1)mGmAmU |
| | | AUGUGCCGCAACGCU | | GUGCmCGmCAAmCGCUCUmGmCC |
| | | CUGCCGGCAUCGUU | | (L1)GGCAUCG*mU*mU |
| G023071 | 619 | CUUCACCAGGAGAAG | 719 | mC*mU*mU*mCACCAGGAGAAGCC |
| | | CCGUCACACGUUGUA | | GUCACACmGUUGmUmAmGmCUCC |
| | | GCUCCCUCCGUUGCU | | CmU(L1)mCmCGUUmGmCUAmCAAU |
| | | ACAAUAAGGCCGUCG | | *AAGmGmCCmGmUmC(L1)mGmAmU |
| | | AUGUGCCGCAACGCU | | GUGCmCGmCAAmCGCUCUmGmCC |
| | | CUGCCGGCAUCGUU | | (L1)GGCAUCG*mU*mU |
| G023072 | 620 | CUUCACCAGGAGAAG | 720 | mC*mU*mU*CmACCAGGAGAAGCC |
| | | CCGUCACACGUUGUA | | GUCACACmGUUGmUmAmGmCUCC |
| | | GCUCCCUCCGUUGCU | | CmU(L1)mCmCGUUmGmCUAmCAAU |
| | | ACAAUAAGGCCGUCG | | *AAGmGmCCmGmUmC(L1)mGmAmU |
| | | AUGUGCCGCAACGCU | | GUGCmCGmCAAmCGCUCUmGmCC |
| | | CUGCCGGCAUCGUU | | (L1)GGCAUCG*mU*mU |
| G023073 | 621 | CUUCACCAGGAGAAG | 721 | mC*mU*mU*CACCAmGGAGAAGCC |
| | | CCGUCACACGUUGUA | | GUCACACmGUUGmUmAmGmCUCC |
| | | GCUCCCUCCGUUGCU | | CmU(L1)mCmCGUUmGmCUAmCAAU |
| | | ACAAUAAGGCCGUCG | | *AAGmGmCCmGmUmC(L1)mGmAmU |
| | | AUGUGCCGCAACGCU | | GUGCmCGmCAAmCGCUCUmGmCC |
| | | CUGCCGGCAUCGUU | | (L1)GGCAUCG*mU*mU |
| G023074 | 622 | CUUCACCAGGAGAAG | 722 | mC*mU*mU*CACCAGGmAGAAGCC |
| | | CCGUCACACGUUGUA | | GUCACACmGUUGmUmAmGmCUCC |
| | | GCUCCCUCCGUUGCU | | CmU(L1)mCmCGUUmGmCUAmCAAU |
| | | ACAAUAAGGCCGUCG | | *AAGmGmCCmGmUmC(L1)mGmAmU |
| | | AUGUGCCGCAACGCU | | GUGCmCGmCAAmCGCUCUmGmCC |
| | | CUGCCGGCAUCGUU | | (L1)GGCAUCG*mU*mU |
| G023075 | 623 | CUUCACCAGGAGAAG | 723 | mC*mU*mU*CACCAGGAGmAAGCC |
| | | CCGUCACACGUUGUA | | GUCACACmGUUGmUmAmGmCUCC |
| | | GCUCCCUCCGUUGCU | | CmU(L1)mCmCGUUmGmCUAmCAAU |
| | | ACAAUAAGGCCGUCG | | *AAGmGmCCmGmUmC(L1)mGmAmU |
| | | AUGUGCCGCAACGCU | | GUGCmCGmCAAmCGCUCUmGmCC |
| | | CUGCCGGCAUCGUU | | (L1)GGCAUCG*mU*mU |
| G023076 | 624 | CUUCACCAGGAGAAG | 724 | mC*mU*mU*CACCAGGAGAAGCCm |
| | | CCGUCACACGUUGUA | | GUCACACmGUUGmUmAmGmCUCC |
| | | GCUCCCUCCGUUGCU | | CmU(L1)mCmCGUUmGmCUAmCAAU |
| | | ACAAUAAGGCCGUCG | | *AAGmGmCCmGmUmC(L1)mGmAmU |
| | | AUGUGCCGCAACGCU | | GUGCmCGmCAAmCGCUCUmGmCC |
| | | CUGCCGGCAUCGUU | | (L1)GGCAUCG*mU*mU |
| G023077 | 625 | CUUCACCAGGAGAAG | 725 | mC*mU*mU*CACCAGGAGAAGCCG |
| | | CCGUCACACGUUGUA | | UCAmCACmGUUGmUmAmGmCUCC |
| | | GCUCCCUCCGUUGCU | | CmU(L1)mCmCGUUmGmCUAmCAAU |
| | | ACAAUAAGGCCGUCG | | *AAGmGmCCmGmUmC(L1)mGmAmU |
| | | AUGUGCCGCAACGCU | | GUGCmCGmCAAmCGCUCUmGmCC |
| | | CUGCCGGCAUCGUU | | (L1)GGCAUCG*mU*mU |
| G023078 | 626 | CUUCACCAGGAGAAG | 726 | mC*mU*mU*CmACCmAmGGmAGmA |
| | | CCGUCACACGUUGUA | | AGCCmGUCAmCACmGUUGmUmAm |
| | | GCUCCCUCCGUUGCU | | GmCUCCCmU(L1)mCmCGUUmGmCU |
| | | ACAAUAAGGCCGUCG | | AmCAAU*AAGmGmCCmGmUmC(L1) |
| | | AUGUGCCGCAACGCU | | mGmAmUGUGCmCGmCAAmCGCUC |
| | | CUGCCGGCAUCGUU | | UmGmCC(L1)GGCAUCG*mU*mU |
| G023079 | 627 | CUUCACCAGGAGAAG | 727 | mC*mU*mU*mCACCmAmGGmAGmA |
| | | CCGUCACACGUUGUA | | AGCCmGUCAmCACmGUUGmUmAm |
| | | GCUCCCUCCGUUGCU | | GmCUCCCmU(L1)mCmCGUUmGmCU |
| | | ACAAUAAGGCCGUCG | | AmCAAU*AAGmGmCCmGmUmC(L1) |
| | | AUGUGCCGCAACGCU | | mGmAmUGUGCmCGmCAAmCGCUC |
| | | CUGCCGGCAUCGUU | | UmGmCC(L1)GGCAUCG*mU*mU |
| G023080 | 628 | CUUCACCAGGAGAAG | 728 | mC*mU*mU*mCmACCAmGGmAGmA |
| | | CCGUCACACGUUGUA | | AGCCmGUCAmCACmGUUGmUmAm |
| | | GCUCCCUCCGUUGCU | | GmCUCCCmU(L1)mCmCGUUmGmCU |
| | | ACAAUAAGGCCGUCG | | AmCAAU*AAGmGmCCmGmUmC(L1) |
| | | AUGUGCCGCAACGCU | | mGmAmUGUGCmCGmCAAmCGCUC |
| | | CUGCCGGCAUCGUU | | UmGmCC(L1)GGCAUCG*mU*mU |
| G023081 | 629 | CUUCACCAGGAGAAG | 729 | mC*mU*mU*mCmACCmAGGmAGmA |
| | | CCGUCACACGUUGUA | | AGCCmGUCAmCACmGUUGmUmAm |
| | | GCUCCCUCCGUUGCU | | GmCUCCCmU(L1)mCmCGUUmGmCU |
| | | ACAAUAAGGCCGUCG | | AmCAAU*AAGmGmCCmGmUmC(L1) |
| | | AUGUGCCGCAACGCU | | mGmAmUGUGCmCGmCAAmCGCUC |
| | | CUGCCGGCAUCGUU | | UmGmCC(L1)GGCAUCG*mU*mU |
| G023082 | 630 | CUUCACCAGGAGAAG | 730 | mC*mU*mU*mCmACCmAmGGAGmA |
| | | CCGUCACACGUUGUA | | AGCCmGUCAmCACmGUUGmUmAm |
| | | GCUCCCUCCGUUGCU | | GmCUCCCmU(L1)mCmCGUUmGmCU |
| | | ACAAUAAGGCCGUCG | | AmCAAU*AAGmGmCCmGmUmC(L1) |
| | | AUGUGCCGCAACGCU | | mGmAmUGUGCmCGmCAAmCGCUC |
| | | CUGCCGGCAUCGUU | | UmGmCC(L1)GGCAUCG*mU*mU |
| G023083 | 631 | CUUCACCAGGAGAAG | 731 | mC*mU*mU*mCmACCmAmGGmAGA |
| | | CCGUCACACGUUGUA | | AGCCmGUCAmCACmGUUGmUmAm |
| | | GCUCCCUCCGUUGCU | | GmCUCCCmU(L1)mCmCGUUmGmCU |
| | | ACAAUAAGGCCGUCG | | AmCAAU*AAGmGmCCmGmUmC(L1) |
| | | AUGUGCCGCAACGCU | | mGmAmUGUGCmCGmCAAmCGCUC |
| | | CUGCCGGCAUCGUU | | UmGmCC(L1)GGCAUCG*mU*mU |
| G023084 | 632 | CUUCACCAGGAGAAG | 732 | mC*mU*mU*mCmACCmAmGGmAGm |
| | | CCGUCACACGUUGUA | | AAGCCGUCAmCACmGUUGmUmAm |
| | | GCUCCCUCCGUUGCU | | GmCUCCCmU(L1)mCmCGUUmGmCU |
| | | ACAAUAAGGCCGUCG | | AmCAAU*AAGmGmCCmGmUmC(L1) |
| | | AUGUGCCGCAACGCU | | mGmAmUGUGCmCGmCAAmCGCUC |
| | | CUGCCGGCAUCGUU | | UmGmCC(L1)GGCAUCG*mU*mU |
| G023085 | 633 | CUUCACCAGGAGAAG | 733 | mC*mU*mU*mCmACCmAmGGmAGm |
| | | CCGUCACACGUUGUA | | AAGCCmGUCACACmGUUGmUmAm |
| | | GCUCCCUCCGUUGCU | | GmCUCCCmU(L1)mCmCGUUmGmCU |
| | | ACAAUAAGGCCGUCG | | AmCAAU*AAGmGmCCmGmUmC(L1) |
| | | AUGUGCCGCAACGCU | | mGmAmUGUGCmCGmCAAmCGCUC |
| | | CUGCCGGCAUCGUU | | UmGmCC(L1)GGCAUCG*mU*mU |
| G023103 | 634 | CCAAGUGUCUUCCAG | 734 | CCAAGUGUCUUCCAGUACGAUUUG |
| | | UACGAUUUGGUUGUA | | mGUUGmUmAmGmCUCCCmU(L1)mC |
| | | GCUCCCUCCGUUGCU | | mCGUUmGmCUAmCAAU*AAGmGm |
| | | ACAAUAAGGCCGUCG | | CCmGmUmC(L1)mGmAmUGUGCmCG |
| | | AUGUGCCGCAACGCU | | mCAAmCGCUCUmGmCC(L1)GGCAU |
| | | CUGCCGGCAUCGUU | | CG*mU*mU |
| G023104 | 635 | CCAAGUGUCUUCCAG | 735 | mC*mC*mA*AGUGUCUUCCAGUAC |
| | | UACGAUUUGGUUGUA | | GAUUUGmGUUGmUmAmGmCUCCC |
| | | GCUCCCUCCGUUGCU | | mU(L1)mCmCGUUmGmCUAmCAAU* |
| | | ACAAUAAGGCCGUCG | | AAGmGmCCmGmUmC(L1)mGmAmU |
| | | AUGUGCCGCAACGCU | | GUGCmCGmCAAmCGCUCUmGmCC |
| | | CUGCCGGCAUCGUU | | (L1)GGCAUCG*mU*mU |
| G023105 | 636 | CCAAGUGUCUUCCAG | 736 | mC*mC*mA*mAGUGUCUUCCAGUA |
| | | UACGAUUUGGUUGUA | | CGAUUUGmGUUGmUmAmGmCUCC |
| | | GCUCCCUCCGUUGCU | | CmU(L1)mCmCGUUmGmCUAmCAAU |
| | | ACAAUAAGGCCGUCG | | *AAGmGmCCmGmUmC(L1)mGmAmU |
| | | AUGUGCCGCAACGCU | | GUGCmCGmCAAmCGCUCUmGmCC |
| | | CUGCCGGCAUCGUU | | (L1)GGCAUCG*mU*mU |
| G023106 | 637 | CCAAGUGUCUUCCAG | 737 | mC*mC*mA*AmGUGUCUUCCAGUA |
| | | UACGAUUUGGUUGUA | | CGAUUUGmGUUGmUmAmGmCUCC |
| | | GCUCCCUCCGUUGCU | | CmU(L1)mCmCGUUmGmCUAmCAAU |
| | | ACAAUAAGGCCGUCG | | *AAGmGmCCmGmUmC(L1)mGmAmU |
| | | AUGUGCCGCAACGCU | | GUGCmCGmCAAmCGCUCUmGmCC |
| | | CUGCCGGCAUCGUU | | (L1)GGCAUCG*mU*mU |
| G023107 | 638 | CCAAGUGUCUUCCAG | 738 | mC*mC*mA*AGUGUmCUUCCAGUA |
| | | UACGAUUUGGUUGUA | | CGAUUUGmGUUGmUmAmGmCUCC |
| | | GCUCCCUCCGUUGCU | | CmU(L1)mCmCGUUmGmCUAmCAAU |
| | | ACAAUAAGGCCGUCG | | *AAGmGmCCmGmUmC(L1)mGmAmU |
| | | AUGUGCCGCAACGCU | | GUGCmCGmCAAmCGCUCUmGmCC |
| | | CUGCCGGCAUCGUU | | (L1)GGCAUCG*mU*mU |
| G023108 | 639 | CCAAGUGUCUUCCAG | 739 | mC*mC*mA*AGUGUCUmUCCAGUA |
| | | UACGAUUUGGUUGUA | | CGAUUUGmGUUGmUmAmGmCUCC |
| | | GCUCCCUCCGUUGCU | | CmU(L1)mCmCGUUmGmCUAmCAAU |
| | | ACAAUAAGGCCGUCG | | *AAGmGmCCmGmUmC(L1)mGmAmU |
| | | AUGUGCCGCAACGCU | | GUGCmCGmCAAmCGCUCUmGmCC |
| | | CUGCCGGCAUCGUU | | (L1)GGCAUCG*mU*mU |
| G023109 | 640 | CCAAGUGUCUUCCAG | 740 | mC*mC*mA*AGUGUCUUCmCAGUA |
| | | UACGAUUUGGUUGUA | | CGAUUUGmGUUGmUmAmGmCUCC |
| | | GCUCCCUCCGUUGCU | | CmU(L1)mCmCGUUmGmCUAmCAAU |
| | | ACAAUAAGGCCGUCG | | *AAGmGmCCmGmUmC(L1)mGmAmU |
| | | AUGUGCCGCAACGCU | | GUGCmCGmCAAmCGCUCUmGmCC |
| | | CUGCCGGCAUCGUU | | (L1)GGCAUCG*mU*mU |
| G023110 | 641 | CCAAGUGUCUUCCAG | 741 | mC*mC*mA*AGUGUCUUCCAGUAm |
| | | UACGAUUUGGUUGUA | | CGAUUUGmGUUGmUmAmGmCUCC |
| | | GCUCCCUCCGUUGCU | | CmU(Ll)mCmCGUUmGmCUAmCAAU |
| | | ACAAUAAGGCCGUCG | | *AAGmGmCCmGmUmC(L1)mGmAmU |
| | | AUGUGCCGCAACGCU | | GUGCmCGmCAAmCGCUCUmGmCC |
| | | CUGCCGGCAUCGUU | | (L1)GGCAUCG*mU*mU |
| G023111 | 642 | CCAAGUGUCUUCCAG | 742 | mC*mC*mA*AGUGUCUUCCAGUAC |
| | | UACGAUUUGGUUGUA | | GAUmUUGmGUUGmUmAmGmCUCC |
| | | GCUCCCUCCGUUGCU | | CmU(L1)mCmCGUUmGmCUAmCAAU |
| | | ACAAUAAGGCCGUCG | | *AAGmGmCCmGmUmC(L1)mGmAmU |
| | | AUGUGCCGCAACGCU | | GUGCmCGmCAAmCGCUCUmGmCC |
| | | CUGCCGGCAUCGUU | | (L1)GGCAUCG*mU*mU |
| G023112 | 643 | CCAAGUGUCUUCCAG | 743 | mC*mC*mA*AmGUGmUmCUmUCmC |
| | | UACGAUUUGGUUGUA | | AGUAmCGAUmUUGmGUUGmUmAm |
| | | GCUCCCUCCGUUGCU | | GmCUCCCmU(L1)mCmCGUUmGmCU |
| | | ACAAUAAGGCCGUCG | | AmCAAU*AAGmGmCCmGmUmC(L1) |
| | | AUGUGCCGCAACGCU | | mGmAmUGUGCmCGmCAAmCGCUC |
| | | CUGCCGGCAUCGUU | | UmGmCC(L1)GGCAUCG*mU*mU |
| G023113 | 644 | CCAAGUGUCUUCCAG | 744 | mC*mC*mA*mAGUGmUmCUmUCmC |
| | | UACGAUUUGGUUGUA | | AGUAmCGAUmUUGmGUUGmUmAm |
| | | GCUCCCUCCGUUGCU | | GmCUCCCmU(L1)mCmCGUUmGmCU |
| | | ACAAUAAGGCCGUCG | | AmCAAU*AAGmGmCCmGmUmC(L1) |
| | | AUGUGCCGCAACGCU | | mGmAmUGUGCmCGmCAAmCGCUC |
| | | CUGCCGGCAUCGUU | | UmGmCC(L1)GGCAUCG*mU*mU |
| G023114 | 645 | CCAAGUGUCUUCCAG | 745 | mC*mC*mA*mAmGUGUmCUmUCmC |
| | | UACGAUUUGGUUGUA | | AGUAmCGAUmUUGmGUUGmUmAm |
| | | GCUCCCUCCGUUGCU | | GmCUCCCmU(L1)mCmCGUUmGmCU |
| | | ACAAUAAGGCCGUCG | | AmCAAU*AAGmGmCCmGmUmC(L1) |
| | | AUGUGCCGCAACGCU | | mGmAmUGUGCmCGmCAAmCGCUC |
| | | CUGCCGGCAUCGUU | | UmGmCC(L1)GGCAUCG*mU*mU |
| G023115 | 646 | CCAAGUGUCUUCCAG | 746 | mC*mC*mA*mAmGUGmUCUmUCmC |
| | | UACGAUUUGGUUGUA | | AGUAmCGAUmUUGmGUUGmUmAm |
| | | GCUCCCUCCGUUGCU | | GmCUCCCmU(L1)mCmCGUUmGmCU |
| | | ACAAUAAGGCCGUCG | | AmCAAU*AAGmGmCCmGmUmC(L1) |
| | | AUGUGCCGCAACGCU | | mGmAmUGUGCmCGmCAAmCGCUC |
| | | CUGCCGGCAUCGUU | | UmGmCC(L1)GGCAUCG*mU*mU |
| G023116 | 647 | CCAAGUGUCUUCCAG | 747 | mC*mC*mA*mAmGUGmUmCUUCmC |
| | | UACGAUUUGGUUGUA | | AGUAmCGAUmUUGmGUUGmUmAm |
| | | GCUCCCUCCGUUGCU | | GmCUCCCmU(L1)mCmCGUUmGmCU |
| | | ACAAUAAGGCCGUCG | | AmCAAU*AAGmGmCCmGmUmC(L1) |
| | | AUGUGCCGCAACGCU | | mGmAmUGUGCmCGmCAAmCGCUC |
| | | CUGCCGGCAUCGUU | | UmGmCC(L1)GGCAUCG*mU*mU |
| G023117 | 648 | CCAAGUGUCUUCCAG | 748 | mC*mC*mA*mAmGUGmUmCUmUCC |
| | | UACGAUUUGGUUGUA | | AGUAmCGAUmUUGmGUUGmUmAm |
| | | GCUCCCUCCGUUGCU | | GmCUCCCmU(L1)mCmCGUUmGmCU |
| | | ACAAUAAGGCCGUCG | | AmCAAU*AAGmGmCCmGmUmC(L1) |
| | | AUGUGCCGCAACGCU | | mGmAmUGUGCmCGmCAAmCGCUC |
| | | CUGCCGGCAUCGUU | | UmGmCC(L1)GGCAUCG*mU*mU |
| G023118 | 649 | CCAAGUGUCUUCCAG | 749 | mC*mC*mA*mAmGUGmUmCUmUCm |
| | | UACGAUUUGGUUGUA | | CAGUACGAUmUUGmGUUGmUmAm |
| | | GCUCCCUCCGUUGCU | | GmCUCCCmU(L1)mCmCGUUmGmCU |
| | | ACAAUAAGGCCGUCG | | AmCAAU*AAGmGmCCmGmUmC(L1) |
| | | AUGUGCCGCAACGCU | | mGmAmUGUGCmCGmCAAmCGCUC |
| | | CUGCCGGCAUCGUU | | UmGmCC(L1)GGCAUCG*mU*mU |
| G023119 | 650 | CCAAGUGUCUUCCAG | 750 | mC*mC*mA*mAmGUGmUmCUmUCm |
| | | UACGAUUUGGUUGUA | | CAGUAmCGAUUUGmGUUGmUmAm |
| | | GCUCCCUCCGUUGCU | | GmCUCCCmU(L1)mCmCGUUmGmCU |
| | | ACAAUAAGGCCGUCG | | AmCAAU*AAGmGmCCmGmUmC(L1) |
| | | AUGUGCCGCAACGCU | | mGmAmUGUGCmCGmCAAmCGCUC |
| | | CUGCCGGCAUCGUU | | UmGmCC(L1)GGCAUCG*mU*mU |
| G023121 | 651 | CUUCACCAAGAGAAG | 751 | mC*mU*mU*CACCmAAGAGAAGCC |
| | | CCGUCACACGUUGUA | | GUCACACmGUUGmUmAmGmCUCC |
| | | GCUCCCUCCGUUGCU | | CmU(L1)mCmCGUUmGmCUAmCAAU |
| | | ACAAUAAGGCCGUCG | | *AAGmGmCCmGmUmC(L1)mGmAmU |
| | | AUGUGCCGCAACGCU | | GUGCmCGmCAAmCGCUCUmGmCC |
| | | CUGCCGGCAUCGUU | | (L1)GGCAUCG*mU*mU |
| G023122 | 652 | CCAAGUGUCUUCCAG | 752 | mC*mC*mA*AGUGmUCUUCCAGUA |
| | | UACGAUUUGGUUGUA | | CGAUUUGmGUUGmUmAmGmCUCC |
| | | GCUCCCUCCGUUGCU | | CmU(L1)mCmCGUUmGmCUAmCAAU |
| | | ACAAUAAGGCCGUCG | | *AAGmGmCCmGmUmC(L1)mGmAmU |
| | | AUGUGCCGCAACGCU | | GUGCmCGmCAAmCGCUCUmGmCC |
| | | CUGCCGGCAUCGUU | | (L1)GGCAUCG*mU*mU |
| G023413 | 653 | CCAAGUGUCUUCCAG | 753 | mC*mC*mA*mAmGUGmUmCUmUCm |
| | | UACGAUUUGGUUGUA | | CAGUAmCGAUmUUGmGUUGmUmA |
| | | GCUCCCUUGCACCGU | | mGmCUCCCmUmUmG(L1)mCmAmCm |
| | | UGCUACAAUAAGGCC | | CGUUmGmCUAmCAAU*AAGmGmCC |
| | | GUCGAUGUGCCGCAA | | mGmUmC(L1)mGmAmUGUGCmCGmC |
| | | CGCUCUGCCGGCAUC | | AAmCGCUCUmGmCC(L1)GGCAUCG |
| | | GUU | | mU*mU |
| G023414 | 654 | CCAAGUGUCUUCCAG | 754 | mC*mC*mA*mAmGUGmUmCUmUCm |
| | | UACGAUUUGGUUGUA | | CAGUAmCGAUmUUGmGUUGmUmA |
| | | GCUCCCUGCCCGUUG | | mGmCUCCCmUmG(L1)mCmCmCGUU |
| | | CUACAAUAAGGCCGU | | mGmCUAmCAAU*AAGmGmCCmGm |
| | | CGAUGUGCCGCAACG | | UmC(L1)mGmAmUGUGCmCGmCAAm |
| | | CUCUGCCGGCAUCGU | | CGCUCUmGmCC(L1)GGCAUCGmU* |
| | | U | | mU |
| G023415 | 655 | CCAAGUGUCUUCCAG | 755 | mC*mC*mA*mAmGUGmUmCUmUCm |
| | | UACGAUUUGGUUGUA | | CAGUAmCGAUmUUGmGUUGmUmA |
| | | GCUCCCGCCGUUGCU | | mGmCUCCCmG(L1)mCmCGUUmGmC |
| | | ACAAUAAGGCCGUCG | | UAmCAAU*AAGmGmCCmGmUmC(L1) |
| | | AUGUGCCGCAACGCU | | mGmAmUGUGCmCGmCAAmCGCU |
| | | CUGCCGGCAUCGUU | | CUmGmCC(L1)GGCAUCGmU*mU |
| G023416 | 656 | CCAAGUGUCUUCCAG | 756 | mC*mC*mA*mAmGUGmUmCUmUCm |
| | | UACGAUUUGGUUGUA | | CAGUAmCGAUmUUGmGUUGmUmA |
| | | GCUCCGCGUUGCUAC | | mGmCUCCmG(L1)mCGUUmGmCUAm |
| | | AAUAAGGCCGUCGAU | | CAAU*AAGmGmCCmGmUmC(L1)mG |
| | | GUGCCGCAACGCUCU | | mAmUGUGCmCGmCAAmCGCUCUm |
| | | GCCGGCAUCGUU | | GmCC(L1)GGCAUCGmU*mU |
| G023417 | 657 | CCAAGUGUCUUCCAG | 757 | mC*mC*mA*mAmGUGmUmCUmUCm |
| | | UACGAUUUGGUUGUA | | CAGUAmCGAUmUUGmGUUGmUmA |
| | | GCUCCGUUGCUACAA | | mGmCUCC(L1)GUUmGmCUAmCAAU |
| | | UAAGGCCGUCGAUGU | | *AAGmGmCCmGmUmC(L1)mGmAmU |
| | | GCCGCAACGCUCUGC | | GUGCmCGmCAAmCGCUCUmGmCC |
| | | CGGCAUCGUU | | (L1)GGCAUCGmU*mU |
| G023418 | 658 | CCAAGUGUCUUCCAG | 758 | mC*mC*mA*mAmGUGmUmCUmUCm |
| | | UACGAUUUGGUUGUA | | CAGUAmCGAUmUUGmGUUGmUmA |
| | | GCUCCCUUGCACCGU | | mGmCUCCCmUmUmG(L1)mCmAmCm |
| | | UGCUACAAUAAGGCC | | CGUUmGmCUAmCAAU*AAGmGmCC |
| | | GUCUAGAUGUGCCGC | | mGmUmCmU(L1)mAmGmAmUGUGC |
| | | AACGCUCUGCCGGCA | | mCGmCAAmCGCUCUmGmCC(L1)GG |
| | | UCGUU | | CAUCGmU*mU |
| G023419 | 659 | CCAAGUGUCUUCCAG | 759 | mC*mC*mA*mAmGUGmUmCUmUCm |
| | | UACGAUUUGGUUGUA | | CAGUAmCGAUmUUGmGUUGmUmA |
| | | GCUCCCUUGCACCGU | | mGmCUCCCmUmUmG(L1)mCmAmCm |
| | | UGCUACAAUAAGGCC | | CGUUmGmCUAmCAAU*AAGmGmCC |
| | | GUCGAUGUGCCGCAA | | mGmUmC(L1)mGmAmUGUGCmCGmC |
| | | CGCUCUGCCUAGGCA | | AAmCGCUCUmGmCCmU(L1)mAGGC |
| | | UCGUU | | AUCGmU*mU |

### III. COMPOSITIONS AND KITS

Compositions comprising any of the gRNAs (e.g., sgRNAs, dgRNAs, or crRNAs) described herein and a carrier, excipient, diluent, or the like are encompassed. In some instances, the excipient or diluent is inert. In some instances, the excipient or diluent is not inert. In some embodiments, a pharmaceutical formulation is provided comprising any of the gRNAs (e.g., sgRNAs, dgRNAs, or crRNAs) described herein and a pharmaceutically acceptable carrier, excipient, diluent, or the like. In some embodiments, the pharmaceutical formulation further comprises an LNP. In some embodiments, the pharmaceutical formulation further comprises a Cas9 protein or an mRNA encoding a Cas9 protein. In some embodiments, the pharmaceutical formulation comprises any one or more of the gRNAs (e.g., sgRNAs, dgRNAs, or crRNAs), an LNP, and a Cas protein or mRNA encoding a Cas protein. In some embodiments, the Cas protein is a monomeric Cas protein, e.g., a Cas9 protein. In some embodiments, the Cas protein includes multiplel subunits.

Also provided are kits comprising one or more gRNAs (e.g., sgRNAs, dgRNAs, or crRNAs), compositions, or pharmaceutical formulations described herein. In some embodiments, a kit further comprises one or more of a solvent, solution, buffer, each separate from the composition or pharmaceutical formulation, instructions, or desiccant.

Compositions Comprising an RNA-Guided DNA Binding Agent or Nucleic Acid Encoding RNA-Guided DNA Binding Agent

In some embodiments, compositions or pharmaceutical formulations are provided comprising at least one gRNA (e.g., sgRNA, dgRNA, or crRNA) described herein and an RNA-guided DNA binding agent or a nucleic acid (e.g., an mRNA) encoding an RNA-guided DNA binding agent. In some embodiments, the RNA-guided DNA binding agent is a Cas protein. In some embodiments, the gRNA together with a Cas protein or nucleic acid (e.g., mRNA) encoding Cas protein is called a Cas RNP. In some embodiments, the RNA-guided DNA binding agent is one that functions with the gRNA to direct an RNA-guided DNA binding agent to a target nucleic acid sequence. In some embodiments, the RNA-guided DNA binding agent is a Cas protein from the Type-II CRISPR/Cas system. In some embodiments, the Cas protein is Cas9. In some embodiments, the Cas9 protein is a wild type Cas9. In some embodiments, the Cas9 protein is derived from the Streptococcus pyogenes Cas9 protein, e.g., a S. pyogenes Cas9 (SpyCas9). In some embodiments, compositions are provided comprising at least one gRNA and a nuclease or an mRNA encoding a spyCas9. In some embodiments, the Cas9 protein is not derived from S. pyogenes, but functions in the same way as S. pyogenes Cas9 such that gRNA that is specific to S. pyogenes Cas9 will direct the non-S. pyogenes Cas9 to its target site. In some embodiments, the Cas9 protein is derived from the Staphylococcus aureus Cas9 protein, e.g., a SauCas9. In some embodiments, compositions are provided comprising at least one gRNA and a nuclease or an mRNA encoding a SauCas9. In some embodiments, the Cas9 protein is derived from the Neisseria meningitidis Cas9 (NmeCas9). In some embodiments, compositions are provided comprising at least one gRNA and a nuclease or an mRNA encoding an NmeCas9. In some embodiments, the Cas9 protein is not derived from N. meningitidis. In some embodiments, compositions are provided comprising at least one gRNA and a nuclease or an mRNA encoding an NmeCas9. In some embodiments, the Cas induces a double strand break in target DNA. Equivalents of SpyCas9, SauCas9, NmeCas9, and other Cas proteins disclosed herein are encompassed by the embodiments described herein.

RNA-guided DNA binding agents, including Cas9, encompass modified and variants thereof. Modified versions having one catalytic domain, either RuvC or HNH, that is inactive are termed “nickases.” Nickases cut only one strand on the target DNA, thus creating a single-strand break. A single-strand break may also be known as a “nick.” In some embodiments, the compositions and methods comprise nickases. In some embodiments, the compositions and methods comprise a nickase RNA-guided DNA binding agent, such as a nickase Cas, e.g., a nickase Cas9, that induces a nick rather than a double strand break in the target DNA.

In some embodiments, the nuclease, e.g. the RNA-guided DNA binding agent, may be modified to contain only one functional nuclease domain. For example, the RNA-guided DNA binding agent may be modified such that one of the nuclease domains is mutated or fully or partially deleted to reduce its nucleic acid cleavage activity. In some embodiments, a nickase Cas is used having a RuvC domain with reduced activity. In some embodiments, a nickase Cas is used having an inactive RuvC domain. In some embodiments, a nickase Cas is used having an HNH domain with reduced activity. In some embodiments, a nickase Cas is used having an inactive HNH domain.

In some embodiments, a conserved amino acid within an RNA-guided DNA binding agent nuclease domain is substituted to reduce or alter nuclease activity. In some embodiments, a Cas protein may comprise an amino acid substitution in the RuvC or RuvC-like nuclease domain. Exemplary amino acid substitutions in the RuvC or RuvC-like nuclease domain include D10A (based on the S. pyogenes Cas9 protein) or H588A (based on the N. meningitidis Cas9 protein). In some embodiments, the Cas protein may comprise an amino acid substitution in the HNH or HNH-like nuclease domain. Exemplary amino acid substitutions in the HNH or HNH-like nuclease domain include E762A, H840A, N863A, H983A, and D986A (based on the SpyCas9 protein) or D16A (based on the NmeCas9 protein).

In some embodiments, the RNP complex described herein comprises a nickase or an mRNA encoding a nickase and a pair of gRNAs (one or both of which may be sgRNAs) that are complementary to the sense and antisense strands of the target sequence, respectively. In this embodiment, the gRNAs (e.g., sgRNAs) direct the nickase to a target sequence and introduce a double stranded break (DSB) by generating a nick on opposite strands of the target sequence (i.e., double nicking). In some embodiments, use of double nicking may improve specificity and reduce off-target effects. In some embodiments, a nickase RNA-guided DNA binding agent is used together with two separate gRNAs (e.g., sgRNAs) that are selected to be in close proximity to produce a double nick in the target DNA.

In some embodiments, chimeric Cas proteins are used, where one domain or region of the protein is replaced by a portion of a different protein. In some embodiments, a Cas nuclease domain may be replaced with a domain from a different nuclease such as Fok1. In some embodiments, a Cas protein may be a modified nuclease.

In some embodiments, the nuclease, e.g., the RNA-guided DNA binding agent, may be modified to induce a point mutation or base change, e.g., a deamination.

In some embodiments, the Cas protein comprises a fusion protein comprising a Cas nuclease (e.g., Cas9), which is a nickase or is catalytically inactive, linked to a heterologous functional domain. In some embodiments, the Cas protein comprises a fusion protein comprising a catalytically inactive Cas nuclease (e.g., Cas9) linked to a heterologous functional domain (see, e.g., WO2014152432). In some embodiments, the catalytically inactive Cas9 is from S. pyogenes. In some embodiments, the catalytically inactive Cas9 is from N. meningitidis. In some embodiments, the catalytically inactive Cas comprises mutations that inactivate the Cas. In some embodiments, the heterologous functional domain is a domain that modifies gene expression, histones, or DNA. In some embodiments, the heterologous functional domain is a transcriptional activation domain or a transcriptional repressor domain. In some embodiments, the nuclease is a catalytically inactive Cas nuclease, such as dCas9.

In some embodiments, the heterologous functional domain is a deaminase, such as a cytidine deaminase or an adenine deaminase. In certain embodiments, the heterologous functional domain is a C to T base converter (cytidine deaminase), such as an apolipoprotein B mRNA editing enzyme (APOBEC) deaminase. A heterologous functional domain such as a deaminase may be part of a fusion protein with a Cas nuclease having nickase activity or a Cas nuclease that is catalytically inactive.

In some embodiments, the target sequence may be adjacent to a PAM. In some embodiments, the PAM may be adjacent to or within 1, 2, 3, or 4, nucleotides of the 3′ end of the target sequence. The length and the sequence of the PAM may depend on the Cas protein used. For example, the PAM may be selected from a consensus or a particular PAM sequence for a specific Cas9 protein or Cas9 ortholog, including those disclosed in FIG. 1 of Ran et al., Nature 520:186-191 (2015). In some embodiments, the PAM may comprise 2, 3, 4, 5, 6, 7, 8, 9, or 10 nucleotides in length. Non-limiting exemplary PAM sequences include NCC, NGG, NAG, NGA, NGAG, NGCG, NNGRRT, TTN, NGGNG, NG, NAAAAN, NNAAAAW, NNNNACA, GNNNCNNA, and NNNNGATT (wherein N is defined as any nucleotide, and W is defined as either A or T, and R is defined as either A or G). In some embodiments, the PAM sequence may be NGG. In some embodiments, the PAM sequence may be NGGNG. In some embodiments, the PAM sequence may be NNAAAAW.

For example, the PAM may be selected from a consensus or a particular PAM sequence for a specific Nine Cas9 protein or Nine Cas9 ortholog (Edraki et al., Mol. Cell 73:714-726, 2019). In some embodiments, the PAM may comprise 2, 3, 4, 5, 6, 7, 8, 9, or 10 nucleotides in length. Non-limiting exemplary PAM sequences include NCC, N4GAYW, N4GYTT, N4GTCT, NNNNCC(a), NNNNCAAA, (wherein N is defined as any nucleotide, W is defined as either A or T, and R is defined as either A or G; and (a) is a preferred, but not required, A after the second C)). In some embodiments, the PAM sequence may be NCC.

In some embodiments, the heterologous functional domain may facilitate transport of the RNA-guided DNA-binding agent into the nucleus of a cell. For example, the heterologous functional domain may be a nuclear localization signal (NLS). In some embodiments, the RNA-guided DNA-binding agent may be fused with 1-10 NLS(s). In some embodiments, the RNA-guided DNA-binding agent may be fused with 1-5 NLS(s). In some embodiments, the RNA-guided DNA-binding agent may be fused with one NLS. Where one NLS is used, the NLS may be fused at the N-terminus or the C-terminus of the RNA-guided DNA-binding agent sequence. It may also be inserted within the RNA-guided DNA binding agent sequence. In other embodiments, the RNA-guided DNA-binding agent may be fused with more than one NLS. In some embodiments, the RNA-guided DNA-binding agent may be fused with 2, 3, 4, or 5 NLSs. In some embodiments, the RNA-guided DNA-binding agent may be fused with two NLSs. In some embodiments, the NLSs may be fused to the N-terminus of the RNA-guided DNA binding agent sequence. In some embodiments, the NLSs may be fused to only the N-terminus of the RNA-guided DNA binding agent sequence. In some embodiments, the RNA-guided DNA binding agent may have no NLS inserted within the RNA-guided DNA-binding agent sequence. In certain embodiments, may have no NLS C-terminal to the RNA-guided DNA-binding agent sequence.

In some embodiments, the RNA-guided DNA-binding agent may be fused with two NLSs. In certain circumstances, the two NLSs may be the same (e.g., two SV40 NLSs) or different. In some embodiments, the RNA-guided DNA-binding agent is fused to two NLS sequences (e.g., SV40) at the carboxy terminus. In some embodiments, the RNA-guided DNA-binding agent may be fused with two NLSs, one at the N-terminus and one at the C-terminus. In some embodiments, the RNA-guided DNA-binding agent may be fused with 3 NLSs. In some embodiments, the RNA-guided DNA-binding agent may be fused with no NLS. In some embodiments, the NLS may be a monopartite sequence, such as, e.g., the SV40 NLS, PKKKRKV (SEQ ID NO: 16) or PKKKRRV (SEQ ID NO: 17). In some embodiments, the NLS may be a bipartite sequence, such as the NLS of nucleoplasmin, KRPAATKKAGQAKKKK (SEQ ID NO: 18). In a specific embodiment, a single PKKKRKV (SEQ ID NO: 19) NLS may be fused at the C-terminus of the RNA-guided DNA-binding agent. One or more linkers are optionally included at the fusion site.

In some embodiments, the RNA-guided DNA binding agent comprises an amino acid sequence with at least 90%, 93%, 95%, 96%, 97%, 98%, 99%, 99.5%, or 100% identity to any one of SEQ ID NOs:301-313 (as shown in Table 5). In some embodiments, the RNA-guided DNA binding agent comprises a sequence with at least 90%, 93%, 95%, 96%, 97%, 98%, 99%, or 100% identity to any one of SEQ ID NOs: 301-313, 350, and 352-360. In some embodiments, any of the foregoing levels of identity is at least 95%, at least 98%, at least 99%, or 100%.

In some embodiments, the mRNA encoding the RNA-guided DNA binding agent comprises a sequence with at least 90%, 93%, 95%, 96%, 97%, 98%, 99%, or 100% identity to any one of SEQ ID NOs: 321-323, 361, 363-372, and 374-382 as shown in Table 5.

### IV. METHODS OF USE

In some embodiments, any one or more of the gRNAs (e.g., sgRNAs, dgRNAs, or crRNAs), compositions, or pharmaceutical formulations described herein is for use in preparing a medicament for treating or preventing a disease or disorder in a subject.

In some embodiments, the invention comprises a method of treating or preventing a disease or disorder in subject comprising administering any one or more of the gRNAs (e.g., sgRNAs, dgRNAs, or crRNAs), compositions, or pharmaceutical formulations described herein.

In some embodiments, the invention comprises a method or use of modifying a target DNA comprising, administering or delivering any one or more of the gRNAs (e.g., sgRNAs, dgRNAs, or crRNAs), compositions, or pharmaceutical formulations described herein.

In some embodiments, the invention comprises a method or use for modulation of a target gene comprising, administering or delivering any one or more of the gRNAs (e.g., sgRNAs, dgRNAs, or crRNAs), compositions, or pharmaceutical formulations described herein. In some embodiments, the modulation is editing of the target gene. In some embodiments, the modulation is a change in expression of the protein encoded by the target gene. As used herein, a “gene editing” or “genetic modification” is a change at the DNA level, e.g., induced by a gRNA/Cas complex. A gene editing or genetic modification may comprise an insertion, deletion, or substitution (base substitution, e.g., C-to-T, or point mutation), typically within a defined sequence or genomic locus. A genetic modification changes the nucleic acid sequence of the DNA. A genetic modification may be at a single nucleotide position. A genetic modification may be at multiple nucleotides, e.g., 2, 3, 4, 5 or more nucleotides, typically in close proximity to each other, e.g, contiguous nucleotides.

In some embodiments, the method or use results in gene editing. In some embodiments, the method or use results in a double-stranded break within the target gene. In some embodiments, the method or use results in formation of indel mutations during non-homologous end joining of the DSB. In some embodiments, the method or use results in an insertion or deletion of nucleotides in a target gene. In some embodiments, the insertion or deletion of nucleotides in a target gene leads to a frameshift mutation or premature stop codon that results in a non-functional protein. In some embodiments, the insertion or deletion of nucleotides in a target gene leads to a knockdown or elimination of target gene expression. In some embodiments, the method or use comprises homology directed repair of a DSB. In some embodiments, the method or use further comprises delivering to the cell a template, wherein at least a part of the template incorporates into a target DNA at or near a double strand break site induced by the nuclease. In some embodiments, the method or use results in a single strand break within the target gene. In some embodiments, the method or use results in a base change, e.g., by deamination, within the target gene. The gene editing typically occurs within or adjacent to the portion of the target gene with which the spacer sequence forms a duplex.

In some embodiments, the method or use results in gene modulation. In some embodiments, the gene modulation is an increase or decrease in gene expression, a change in methylation state of DNA, or modification of a histone subunit. In some embodiments, the method or use results in increased or decreased expression of the protein encoded by the target gene.

The efficacy of gRNAs (e.g., sgRNAs, dgRNAs, or crRNAs) can be tested in vitro and in vivo. In some embodiments, the invention comprises one or more of the gRNAs (e.g., sgRNAs, dgRNAs, or crRNAs), compositions, or pharmaceutical formulations described herein, wherein the gRNA results in gene modulation when provided to a cell together with a Cas nuclease, e.g., Cas9 or mRNA encoding Cas9. In some embodiments, the efficacy of gRNA can be measured in vitro or in vivo.

In some embodiments, the activity of a Cas RNP comprising a gRNA is compared to the activity of a Cas RNP comprising an unmodified sgRNA or a reference sgRNA lacking modifications present in the sgRNA, such as one or more internal linkers, shortened regions, or YA site substitutions.

In some embodiments, the efficiency of a gRNA in increasing or decreasing target protein expression is determined by measuring the amount of target protein.

In some embodiments, the efficiency of editing with specific gRNAs is determined by the editing present at the target location in the genome following delivery of a Cas nuclease and the gRNA. In some embodiments, the efficiency of editing with specific gRNAs is measured by next-generation sequencing. In some embodiments, the editing percentage of the target region of interest is determined. In some embodiments, the total number of sequence reads with sequence alterations, e.g., insertions or deletions (indels), or base changes with no insertion or deletion, of nucleotides into the target region of interest over the total number of sequence reads is measured following delivery of a gRNA and a Cas nuclease.

In some embodiments, the efficiency of editing with specific gRNAs is measured by the presence of sequence alterations, e.g., insertions or deletions, or base substituition, or point mutation of nucleotides introduced by successful gene editing. In some embodiments, activity of a Cas nuclease and gRNAs is tested in biochemical assays. In some embodiments, activity of a Cas nuclease and gRNAs is tested in a cell-free cleavage assay. In some embodiments, activity of a Cas nuclease and gRNAs is tested in Neuro2A cells. In some embodiments, activity of a Cas nuclease and gRNAs is tested in primary cells, e.g., primary hepatocytes.

In some embodiments, the activity of modified gRNAs is measured after in vivo dosing of LNPs comprising modified gRNAs and Cas protein or mRNA encoding Cas protein.

In some embodiments, in vivo efficacy of a gRNA or composition provided herein is determined by editing efficacy measured in DNA extracted from tissue (e.g., liver tissue) after administration of gRNA and a Cas nuclease.

In some embodiments, activation of the subject's immune response is measured by serum concentrations of cytokine(s) following in vivo dosing of sgRNA together with Cas nuclease mRNA or protein (e.g., formulated in an LNP). In some embodiments, the cytokine is interferon-alpha (IFN-alpha), interleukin 6 (IL-6), monocyte chemotactic protein 1 (MCP-1), or tumor necrosis factor alpha (TNF-alpha).

In some embodiments, administration of Cas RNP or Cas nuclease mRNA together with the modified gRNA (e.g., sgRNA, or dgRNA) produces lower serum concentration(s) of immune cytokines compared to administration of unmodified sgRNA. In some embodiments, the invention comprises methods comprising administering any one of the gRNAs disclosed herein to a subject, wherein the gRNA elicits a lower concentration of immune cytokines in the subject's serum as compared to a control gRNA that is not similarly modified.

### V. DELIVERY OF GUIDE RNA

In some embodiments, the gRNA compositions, compositions, or pharmaceutical formulations disclosed herein, alone or encoded on one or more vectors, are formulated in or administered via a lipid nanoparticle; see e.g., WO2017/173054, the contents of which are hereby incorporated by reference in their entirety.

Lipids; Formulation; Delivery

Disclosed herein are various embodiments using lipid nucleic acid assembly compositions comprising nucleic acids(s), or composition(s) described herein. In some embodiments, the lipid nucleic acid assembly composition comprises a nucleic acid described herein (e.g., a gRNA comprising an internal linker).

As used herein, a “lipid nucleic acid assembly composition” refers to lipid-based delivery compositions, including lipid nanoparticles (LNPs) and lipoplexes. LNP refers to lipid nanoparticles <100 nm. LNPs are formed by precise mixing a lipid component (e.g., in ethanol) with an aqueous nucleic acid component and LNPs are uniform in size. Lipoplexes are particles formed by bulk mixing the lipid and nucleic acid components and are between about 100 nm and 1 micron in size. In certain embodiments the lipid nucleic acid assemblies are LNPs. As used herein, a “lipid nucleic acid assembly” comprises a plurality of (i.e. more than one) lipid molecules physically associated with each other by intermolecular forces. A lipid nucleic acid assembly may comprise a bioavailable lipid having a pKa value of <7.5 or <7. The lipid nucleic acid assemblies are formed by mixing an aqueous nucleic acid-containing solution with an organic solvent-based lipid solution, e.g., 100% ethanol. Suitable solutions or solvents include or may contain: water, PBS, Tris buffer, NaCl, citrate buffer, ethanol, chloroform, diethylether, cyclohexane, tetrahydrofuran, methanol, isopropanol. A pharmaceutically acceptable buffer may optionally be comprised in a pharmaceutical formulation comprising the lipid nucleic acid assemblies, e.g., for an ex vivo therapy. In some embodiments, the aqueous solution comprises a gRNA described herein. In some embodiments, the aqueous solution further comprises an mRNA encoding an RNA-guided DNA binding agent, such as Cas9.

As used herein, lipid nanoparticle (LNP) refers to a particle that comprises a plurality of (i.e., more than one) lipid molecules physically associated with each other by intermolecular forces. The LNPs may be, e.g., microspheres (including unilamellar and multilamellar vesicles, e.g., “liposomes”-lamellar phase lipid bilayers that, in some embodiments, are substantially spherical—and, in more particular embodiments, can comprise an aqueous core, e.g., comprising a substantial portion of RNA molecules), a dispersed phase in an emulsion, micelles, or an internal phase in a suspension. Emulsions, micelles, and suspensions may be suitable compositions for local and/or topical delivery. See also, e.g., WO2017173054A1, the contents of which are hereby incorporated by reference in their entirety. Any LNP known to those of skill in the art to be capable of delivering nucleotides to subjects may be utilized with the guide RNAs described herein.

In some embodiments, the aqueous solution comprises a gRNA described herein. A pharmaceutical formulation comprising the lipid nucleic acid assembly composition may optionally comprise a pharmaceutically acceptable buffer.

In some embodiments, the lipid nucleic acid assembly compositions include an “amine lipid” (sometimes herein or elsewhere described as an “ionizable lipid” or a “biodegradable lipid”), together with an optional “helper lipid”, a “neutral lipid”, and a stealth lipid such as a PEG lipid. In some embodiments, the amine lipids or ionizable lipids are cationic depending on the pH.

### Amine Lipids

In some embodiments, lipid nucleic acid assembly compositions comprise an “amine lipid”, which is, for example an ionizable lipid such as Lipid A or its equivalents, including acetal analogs of Lipid A.

In some embodiments, the amine lipid is Lipid A, which is (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate. Lipid A can be depicted as:

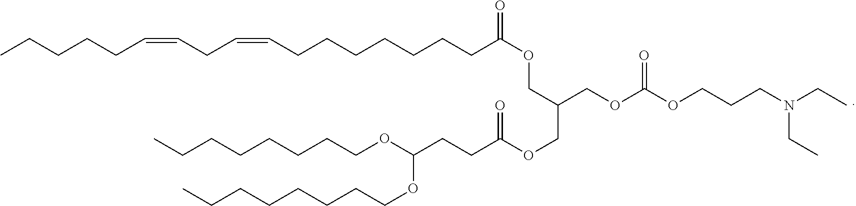 [SMILES: CCCCC/C=C\C/C=C\CCCCCCCC(=O)OCC(COC(=O)CCC(OCCCCCCCC)OCCCCCCCC)COC(=O)OCCCN(CC)CC]

Lipid A may be synthesized according to WO2015/095340 (e.g., pp. 84-86). In some embodiments, the amine lipid is an equivalent to Lipid A.

In some embodiments, an amine lipid is an analog of Lipid A. In some embodiments, a Lipid A analog is an acetal analog of Lipid A. In particular lipid nucleic acid assembly compositions, the acetal analog is a C4-C12 acetal analog. In some embodiments, the acetal analog is a C5-C12 acetal analog. In additional embodiments, the acetal analog is a C5-C10 acetal analog. In further embodiments, the acetal analog is chosen from a C4, C5, C6, C7, C9, C10, C11, and C12 acetal analog.

Amine lipids and other “biodegradable lipids” suitable for use in the lipid nucleic acid assemblies described herein are biodegradable in vivo or ex vivo. The amine lipids have low toxicity (e.g., are tolerated in animal models without adverse effect in amounts of greater than or equal to 10 mg/kg). In some embodiments, lipid nucleic acid assemblies comprising an amine lipid include those where at least 75% of the amine lipid is cleared from the plasma or the engineered cell within 8, 10, 12, 24, or 48 hours, or 3, 4, 5, 6, 7, or 10 days. In some embodiments, lipid nucleic acid assemblies comprising an amine lipid include those where at least 50% of the nucleic acid, e.g., mRNA or gRNA, is cleared from the plasma within 8, 10, 12, 24, or 48 hours, or 3, 4, 5, 6, 7, or 10 days. In some embodiments, lipid nucleic acid assemblies comprising an amine lipid include those where at least 50% of the lipid nucleic acid assembly is cleared from the plasma within 8, 10, 12, 24, or 48 hours, or 3, 4, 5, 6, 7, or 10 days, for example by measuring a lipid (e.g. an amine lipid), nucleic acid, e.g., RNA/mRNA, or other component. In some embodiments, lipid-encapsulated versus free lipid, RNA, or nucleic acid component of the lipid nucleic acid assembly is measured.

Biodegradable lipids include, for example the biodegradable lipids of WO/2020/219876, WO/2020/118041, WO/2020/072605, WO/2019/067992, WO/2017/173054, WO2015/095340, and WO2014/136086, and LNPs include LNP compositions described therein, the lipids and compositions of which are hereby incorporated by reference.

Lipid clearance may be measured as described in literature. See Maier, M. A., et al. Biodegradable Lipids Enabling Rapidly Eliminated Lipid Nanoparticles for Systemic Delivery of RNAi Therapeutics. Mol. Ther. 2013, 21(8), 1570-78 (“Maier”). For example, in Maier, LNP-siRNA systems containing luciferases-targeting siRNA were administered to six- to eight-week old male C57Bl/6 mice at 0.3 mg/kg by intravenous bolus injection via the lateral tail vein. Blood, liver, and spleen samples were collected at 0.083, 0.25, 0.5, 1, 2, 4, 8, 24, 48, 96, and 168 hours post-dose. Mice were perfused with saline before tissue collection and blood samples were processed to obtain plasma. All samples were processed and analyzed by LC-MS. Further, Maier describes a procedure for assessing toxicity after administration of LNP-siRNA formulations. For example, a luciferase-targeting siRNA was administered at 0, 1, 3, 5, and 10 mg/kg (5 animals/group) via single intravenous bolus injection at a dose volume of 5 mL/kg to male Sprague-Dawley rats. After 24 hours, about 1 mL of blood was obtained from the jugular vein of conscious animals and the serum was isolated. At 72 hours post-dose, all animals were euthanized for necropsy. Assessments of clinical signs, body weight, serum chemistry, organ weights and histopathology were performed. Although Maier describes methods for assessing siRNA-LNP formulations, these methods may be applied to assess clearance, pharmacokinetics, and toxicity of administration of lipid nucleic acid assembly compositions of the present disclosure.

Ionizable and bioavailable lipids for LNP delivery of nucleic acids known in the art are suitable. Lipids may be ionizable depending upon the pH of the medium they are in. For example, in a slightly acidic medium, the lipid, such as an amine lipid, may be protonated and thus bear a positive charge. Conversely, in a slightly basic medium, such as, for example, blood where pH is approximately 7.35, the lipid, such as an amine lipid, may not be protonated and thus bear no charge.

The ability of a lipid to bear a charge is related to its intrinsic pKa. In some embodiments, the amine lipids of the present disclosure may each, independently, have a pKa in the range of from about 5.1 to about 7.4. In some embodiments, the bioavailable lipids of the present disclosure may each, independently, have a pKa in the range of from about 5.1 to about 7.4, such as from about 5.5 to about 6.6, from about 5.6 to about 6.4, from about 5.8 to about 6.2, or from about 5.8 to about 6.5. For example, the amine lipids of the present disclosure may each, independently, have a pKa in the range of from about 5.8 to about 6.5. Lipids with a pKa ranging from about 5.1 to about 7.4 are effective for delivery of cargo in vivo, e.g. to the liver. Further, it has been found that lipids with a pKa ranging from about 5.3 to about 6.4 are effective for delivery in vivo, e.g. to tumors. See, e.g., WO2014/136086.

Additional Lipids

“Neutral lipids” suitable for use in a lipid nucleic acid assembly composition of the disclosure include, for example, a variety of neutral, uncharged or zwitterionic lipids. Examples of neutral phospholipids suitable for use in the present disclosure include, but are not limited to, 5-heptadecylbenzene-1,3-diol (resorcinol), dipalmitoylphosphatidylcholine (DPPC), distearoylphosphatidylcholine (DSPC), pohsphocholine (DOPC), dimyristoylphosphatidylcholine (DMPC), phosphatidylcholine (PLPC), 1,2-distearoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), lysophosphatidyl choline, dioleoyl phosphatidylethanolamine (DOPE), dilinoleoylphosphatidylcholine distearoylphosphatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine and combinations thereof. In one embodiment, the neutral phospholipid may be selected from the group consisting of distearoylphosphatidylcholine (DSPC) and dimyristoyl phosphatidyl ethanolamine (DMPE). In another embodiment, the neutral phospholipid may be distearoylphosphatidylcholine (DSPC).

“Helper lipids” include steroids, sterols, and alkyl resorcinols. Helper lipids suitable for use in the present disclosure include, but are not limited to, cholesterol, 5-heptadecylresorcinol, and cholesterol hemisuccinate. In one embodiment, the helper lipid may be cholesterol. In one embodiment, the helper lipid may be cholesterol hemisuccinate.

“Stealth lipids” are lipids that alter the length of time the nanoparticles can exist in vivo (e.g., in the blood). Stealth lipids may assist in the formulation process by, for example, reducing particle aggregation and controlling particle size. Stealth lipids used herein may modulate pharmacokinetic properties of the lipid nucleic acid assembly or aid in stability of the nanoparticle ex vivo. Stealth lipids suitable for use in a lipid nucleic acid assembly composition of the disclosure include, but are not limited to, stealth lipids having a hydrophilic head group linked to a lipid moiety. Stealth lipids suitable for use in a lipid nucleic acid assembly composition of the present disclosure and information about the biochemistry of such lipids can be found in Romberg et al., Pharmaceutical Research, Vol. 25, No. 1, 2008, pg. 55-71 and Hoekstra et al., Biochimica et Biophysica Acta 1660 (2004) 41-52. Additional suitable PEG lipids are disclosed, e.g., in WO 2006/007712.

In one embodiment, the hydrophilic head group of stealth lipid comprises a polymer moiety selected from polymers based on PEG. Stealth lipids may comprise a lipid moiety. In some embodiments, the stealth lipid is a PEG lipid.

In one embodiment, a stealth lipid comprises a polymer moiety selected from polymers based on PEG (sometimes referred to as poly(ethylene oxide)), poly(oxazoline), poly(vinyl alcohol), poly(glycerol), poly(N-vinylpyrrolidone), polyaminoacids and poly[N-(2-hydroxypropyl)methacrylamide].

In one embodiment, the PEG lipid comprises a polymer moiety based on PEG (sometimes referred to as poly(ethylene oxide)).

The PEG lipid further comprises a lipid moiety. In some embodiments, the lipid moiety may be derived from diacylglycerol or diacylglycamide, including those comprising a dialkylglycerol or dialkylglycamide group having alkyl chain length independently comprising from about C4 to about C40 saturated or unsaturated carbon atoms, wherein the chain may comprise one or more functional groups such as, for example, an amide or ester. In some embodiments, the alkyl chain length comprises about C10 to C20. The dialkylglycerol or dialkylglycamide group can further comprise one or more substituted alkyl groups. The chain lengths may be symmetrical or asymmetrical.

Unless otherwise indicated, the term “PEG” as used herein means any polyethylene glycol or other polyalkylene ether polymer. In one embodiment, PEG is an optionally substituted linear or branched polymer of ethylene glycol or ethylene oxide. In one embodiment, PEG is unsubstituted. In one embodiment, the PEG is substituted, e.g., by one or more alkyl, alkoxy, acyl, hydroxy, or aryl groups. In one embodiment, the term includes PEG copolymers such as PEG-polyurethane or PEG-polypropylene (see, e.g., J. Milton Harris, Poly(ethylene glycol) chemistry: biotechnical and biomedical applications (1992)); in another embodiment, the term does not include PEG copolymers. In one embodiment, the PEG has a molecular weight of from about 130 to about 50,000, in a sub-embodiment, about 150 to about 30,000, in a sub-embodiment, about 150 to about 20,000, in a sub-embodiment about 150 to about 15,000, in a sub-embodiment, about 150 to about 10,000, in a sub-embodiment, about 150 to about 6,000, in a sub-embodiment, about 150 to about 5,000, in a sub-embodiment, about 150 to about 4,000, in a sub-embodiment, about 150 to about 3,000, in a sub-embodiment, about 300 to about 3,000, in a sub-embodiment, about 1,000 to about 3,000, and in a sub-embodiment, about 1,500 to about 2,500.

In some embodiments, the PEG (e.g., conjugated to a lipid moiety or lipid, such as a stealth lipid), is a “PEG-2K,” also termed “PEG2k” or “PEG 2000,” which has an average molecular weight of about 2,000 daltons. PEG-2K is represented herein by the following formula (I), wherein n is 45, meaning that the number averaged degree of polymerization comprises about 45 subunits

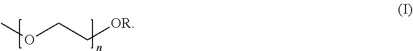

However, other PEG embodiments known in the art may be used, including, e.g., those where the number-averaged degree of polymerization comprises about 23 subunits (n=23), and/or 68 subunits (n=68). In some embodiments, n may range from about 30 to about 60. In some embodiments, n may range from about 35 to about 55. In some embodiments, n may range from about 40 to about 50. In some embodiments, n may range from about 42 to about 48. In some embodiments, n may be 45. In some embodiments, R may be selected from H, substituted alkyl, and unsubstituted alkyl. In some embodiments, R may be unsubstituted alkyl. In some embodiments, R may be methyl.

In any of the embodiments described herein, the PEG lipid may be selected from PEG-dilauroylglycerol, PEG-dimyristoylglycerol (PEG-DMG) (e.g., 1,2-dimyristoyl-rac-glycero-3-methylpolyoxyethylene glycol 2000 (PEG2k-DMG) or PEG-DMG (catalog #GM-020 from NOF, Tokyo, Japan)), PEG-dipalmitoylglycerol, PEG-distearoylglycerol (PEG-DSPE) (catalog #DSPE-020CN, NOF, Tokyo, Japan), PEG-dilaurylglycamide, PEG-dimyristylglycamide, PEG-dipalmitoylglycamide, and PEG-distearoylglycamide, PEG-cholesterol (1-[8′-(Cholest-5-en-3[beta]-oxy)carboxamido-3′,6′-dioxaoctanyl]carbamoyl-[omega]-methyl-poly(ethylene glycol), PEG-DMB (3,4-ditetradecoxylbenzyl-[omega]-methyl-poly(ethylene glycol)ether), 1,2-dimyristoyl-sn-glycero-3-phosphoethanolamine-N-[methoxy(polyethylene glycol)-2000] (PEG2k-DMG) (cat. #880150P from Avanti Polar Lipids, Alabaster, Alabama, USA), 1,2-distearoyl-sn-glycero-3-phosphoethanolamine-N-[methoxy(polyethylene glycol)-2000] (PEG2k-DSPE) (cat. #880120C from Avanti Polar Lipids, Alabaster, Alabama, USA), 1,2-distearoyl-sn-glycerol, methoxypolyethylene glycol (PEG2k-DSG; GS-020, NOF Tokyo, Japan), poly(ethylene glycol)-2000-dimethacrylate (PEG2k-DMA), and 1,2-distearyloxypropyl-3-amine-N-[methoxy(polyethylene glycol)-2000] (PEG2k-DSA). In one embodiment, the PEG lipid may be 1,2-dimyristoyl-rac-glycero-3-methylpolyoxyethylene glycol 2000 (PEG2k-DMG). In one embodiment, the PEG lipid may be PEG2k-DMG. In some embodiments, the PEG lipid may be PEG2k-DSG. In one embodiment, the PEG lipid may be PEG2k-DSPE. In one embodiment, the PEG lipid may be PEG2k-DMA. In one embodiment, the PEG lipid may be PEG2k-C-DMA. In one embodiment, the PEG lipid may be compound 5027, disclosed in WO2016/010840 (paragraphs [00240] to [00244]). In one embodiment, the PEG lipid may be PEG2k-DSA. In one embodiment, the PEG lipid may be PEG2k-C11. In some embodiments, the PEG lipid may be PEG2k-C14. In some embodiments, the PEG lipid may be PEG2k-C16. In some embodiments, the PEG lipid may be PEG2k-C18.

LNP Delivery of gRNA

Lipid nanoparticles (LNPs) are a well-known means for delivery of nucleotide and protein cargo, and may be used for delivery of the gRNAs (e.g., sgRNAs, dgRNAs, or crRNAs), compositions, or pharmaceutical formulations disclosed herein. In some embodiments, the LNPs deliver nucleic acid, protein, or nucleic acid together with protein. As used herein, lipid nanoparticle (LNP) refers to a particle that comprises a plurality of (i.e., more than one) lipid molecules physically associated with each other by intermolecular forces. The LNPs may be, e.g., microspheres (including unilamellar and multilamellar vesicles, e.g., “liposomes”-lamellar phase lipid bilayers that, in some embodiments, are substantially spherical and, in more particular embodiments, can comprise an aqueous core, e.g., comprising a substantial portion of RNA molecules), a dispersed phase in an emulsion, micelles, or an internal phase in a suspension (see, e.g., WO2017173054, the contents of which are hereby incorporated by reference in their entirety). Any LNP known to those of skill in the art to be capable of delivering nucleotides to subjects may be utilized.

In some embodiments, the invention comprises a method for delivering any one of the gRNAs (e.g., sgRNAs, dgRNAs, or crRNAs) disclosed herein to a subject, wherein the gRNA is associated with an LNP. In some embodiments, the gRNA/LNP is also associated with a Cas nuclease or a polynucleotide (e.g., mRNA or DNA) encoding a Cas nuclease.

In some embodiments, the invention comprises a composition comprising any one of the gRNAs disclosed and an LNP. In some embodiments, the composition further comprises a Cas9 or a polynucleotide (e.g., mRNA or DNA) encoding Cas9.

In some embodiments, provided herein is a method for delivering any of the guide RNAs described herein to a cell or a population of cells or a subject, including to a cell or population of cells in a subject in vivo, wherein any one or more of the components is associated with an LNP. In some embodiments, the method further comprises an RNA-guided DNA-binding agent (e.g., Cas9 or a polynucleotide (e.g., mRNA or DNA) encoding Cas9).

In some embodiments, provided herein is a composition comprising any of the guide RNAs described herein or donor construct disclosed herein, alone or in combination, with an LNP. In some embodiments, the composition further comprises an RNA-guided DNA-binding agent (e.g., Cas9 or a polynucleotide (e.g., mRNA or DNA) encoding Cas9).

In some embodiments, the LNPs comprise cationic lipids. In some embodiments, the LNPs comprise (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate). In some embodiments, the LNPs comprise molar ratios of a cationic lipid amine to RNA phosphate (N:P) of about 4.5. In some embodiments, the LNPs comprise is nonyl 8-((7,7-bis(octyloxy)heptyl)(2-hydroxyethyl)amino)octanoate. In some embodiments, the LNPs comprise molar ratios of a cationic lipid amine to RNA phosphate (N:P) of about 4.5-6.5. In some embodiments, the LNPs comprise molar ratios of a cationic lipid amine to RNA phosphate (N:P) of about 4.5. In some embodiments, the LNPs comprise molar ratios of a cationic lipid amine to RNA phosphate (N:P) of about 6.0.

In some embodiments, LNPs associated with the gRNAs disclosed herein are for use in preparing a medicament for treating a disease or disorder.

Electroporation is a well-known means for delivery of cargo, and any electroporation methodology may be used for delivery of any one of the gRNAs disclosed herein. In some embodiments, electroporation may be used to deliver any one of the gRNAs disclosed herein and Cas9 or a polynucleotide (e.g., mRNA or DNA) encoding Cas9.

In some embodiments, the invention comprises a method for delivering any one of the gRNAs disclosed herein to an ex vivo cell, wherein the gRNA is associated with an LNP or not associated with an LNP. In some embodiments, the gRNA/LNP or gRNA is also associated with a Cas9 or a polynucleotide (e.g., mRNA or DNA) encoding Cas9. See, e.g., WO2021222287, incorporated herein by reference.

In some embodiments, the vector comprises one or more nucleotide sequence(s) encoding a an mRNA encoding an RNA-guided DNA nuclease, which can be a Cas nuclease, such as Cas9 or Cpf1. In some embodiments, the vector comprises one or more nucleotide sequence(s) encoding a crRNA, a trRNA, and an mRNA encoding an RNA-guided DNA nuclease, which can be a Cas protein, such as, Cas9. In one embodiment, the Cas9 is from Spy Cas9 or NmeCas9. In some embodiments, the nucleotide sequence encoding the crRNA, trRNA, or crRNA and trRNA (which may be a sgRNA) comprises or consists of a guide sequence flanked by all or a portion of a repeat sequence from a naturally-occurring CRISPR/Cas system. The nucleic acid comprising or consisting of the crRNA, trRNA, or crRNA and trRNA may further comprise a vector sequence wherein the vector sequence comprises or consists of nucleic acids that are not naturally found together with the crRNA, trRNA, or crRNA and trRNA.

In some embodiments, the components can be introduced as naked nucleic acid, as nucleic acid complexed with an agent such as a liposome or poloxamer, or they can be delivered by viral vectors (e.g., adenovirus, AAV, herpesvirus, retrovirus, lentivirus). Methods and compositions for non-viral delivery of nucleic acids include electroporation, lipofection, microinjection, biolistics, virosomes, liposomes, immunoliposomes, LNPs, polycation or lipid:nucleic acid conjugates, naked nucleic acid (e.g., naked DNA/RNA), artificial virions, and agent-enhanced uptake of DNA. Sonoporation using, e.g., the Sonitron 2000 system (Rich-Mar) can also be used for delivery of nucleic acids.

This description and exemplary embodiments should not be taken as limiting. For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified.

TABLE 5
Table of Sequences

| | SEQ | |
|---|---|---|
| | ID | |
| Description | NO: | Sequence |
| SpyCas9 | 301 | MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSF |
| amino acid | | FHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQ |
| sequence | | LFEENPINASGVDAKAILSARLSKSRRLENLIAQLPGEKKNGLFGNLIALSLGLTPNFKSNFDLAEDAKLQLSKDTYDDDLDNLLAQIGDQYADLFL |
| | | AAKNLSDAILLSDILRVNTEITKAPLSASMIKRYDEHHQDLTLLKALVRQQLPEKYKEIFFDQSKNGYAGYIDGGASQEEFYKFIKPILEKMDGTEE |
| | | LLVKLNREDLLRKQRTFDNGSIPHQIHLGELHAILRRQEDFYPFLKDNREKIEKILTFRIPYYVGPLARGNSRFAWMTRKSEETITPWNFEEVVDKG |
| | | ASAQSFIERMTNFDKNLPNEKVLPKHSLLYEYFTVYNELTKVKYVTEGMRKPAFLSGEQKKAIVDLLEKTNRKVTVKQLKEDYFKKIECFDSVEISG |
| | | VEDRFNASLGTYHDLLKIIKDKDELDNEENEDILEDIVLTLTLFEDREMIEERLKTYAHLEDDKVMKQLKRRRYTGWGRLSRKLINGIRDKQSGKTI |
| | | LDFLKSDGFANRNFMQLIHDDSLTFKEDIQKAQVSGQGDSLHEHIANLAGSPAIKKGILQTVKVVDELVKVMGRHKPENIVIEMARENQTTQKGQKN |
| | | SRERMKRIEEGIKELGSQILKEHPVENTQLQNEKLYLYYLQNGRDMYVDQELDINRLSDYDVDHIVPQSFLKDDSIDNKVLTRSDKNRGKSDNVPSE |
| | | EVVKKMKNYWRQLLNAKLITQRKFDNLTKAERGGLSELDKAGFIKRQLVETRQITKHVAQILDSRMNTKYDENDKLIREVKVITLKSKLVSDERKDF |
| | | QFYKVREINNYHHAHDAYLNAVVGTALIKKYPKLESEFVYGDYKVYDVRKMIAKSEQEIGKATAKYFFYSNIMNFFKTEITLANGEIRKRPLIETNG |
| | | ETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELL |
| | | GITIMERSSFEKNPIDFLEAKGYKEVKKDLIIKLPKYSLFELENGRKRMLASAGELQKGNELALPSKYVNFLYLASHYEKLKGSPEDNEQKQLFVEQ |
| | | HKHYLDEIIEQISEFSKRVILADANLDKVLSAYNKHRDKPIREQAENIIHLFTLTNLGAPAAFKYFDTTIDRKRYTSTKEVLDATLIHQSITGLYET |
| | | RIDLSQLGGDGGGSPKKKRKV |
| SauCas9 | 302 | MALEAPKKKRKVGSDYKDDDDKKRNYILGLDIGITSVGYGIIDYETRDVIDAGVRLFKEANVENNEGRRSKRGARRLKRRRRHRIQRVKKLLFDYNL |
| amino acid | | LTDHSELSGINPYEARVKGLSQKLSEEEFSAALLHLAKRRGVHNVNEVEEDTGNELSTKEQISRNSKALEEKYVAELQLERLKKDGEVRGSINRFKT |
| sequence | | SDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRD |
| | | ENEKLEYYEKFQIIENVFKQKKKPTLKQIAKEILVNEEDIKGYRVTSTGKPEFTNLKVYHDIKDITARKEIIENAELLDQIAKILTIYQSSEDIQEE |
| | | LTNLNSELTQEEIEQISNLKGYTGTHNLSLKAINLILDELWHTNDNQIAIFNRLKLVPKKVDLSQQKEIPTTLVDDFILSPVVKRSFIQSIKVINAI |
| | | IKKYGLPNDIIIELAREKNSKDAQKMINEMQKRNRQTNERIEEIIRTTGKENAKYLIEKIKLHDMQEGKCLYSLEAIPLEDLLNNPENYEVDHIIPR |
| | | SVSFDNSFNNKVLVKQEENSKKGNRTPFQYLSSSDSKISYETFKKHILNLAKGKGRISKTKKEYLLEERDINRFSVQKDFINRNLVDTRYATRGLMN |
| | | LLRSYFRVNNLDVKVKSINGGFTSFLRRKWKFKKERNKGYKHHAEDALIIANADFIFKEWKKLDKAKKVMENQMFEEKQAESMPEIETEQEYKEIFI |
| | | TPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKN |
| | | PLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEE |
| | | AKKLKKISNQAEFIASFYNNDLIKINGELYRVIGVNNDLLNRIEVNMIDITYREYLENMNDKRPPRIIKTIASKTQSIKKYSTDILGNLYEVKSKKH |
| | | PQIIKKG |
| CdiCas9 | 303 | MKYHVGIDVGTFSVGLAAIEVDDAGMPIKTLSLVSHIHDSGLDPDKIKSAVTRLASSGIARRTRRLYRRKRRRLQQLDKFIQRQGWPVIELEDYSDP |
| amino acid | | LYPWKVRAELAASYIADEKERGEKLSVALRHIARHRGWRNPYAKVSSLYLPDEPSDAFKAIREEIKRASGQPVPETATVGQMVTLCELGTLKLRGEG |
| sequence | | GVLSARLQQSDHAREIQEICRMQEIGQELYRKIIDVVFAAESPKGSASSRVGKDPLQPGKNRALKASDAFQRYRIAALIGNLRVRVDGEKRILSVEE |
| | | KNLVFDHLVNLAPKKEPEWVTIAEILGIDRGQLIGTATMTDDGERAGARPPTHDTNRSIVNSRIAPLVDWWKTASALEQHAMVKALSNAEVDDFDSP |
| | | EGAKVQAFFADLDDDVHAKLDSLHLPVGRAAYSEDTLVRLTRRMLADGVDLYTARLQEFGIEPSWTPPAPRIGEPVGNPAVDRVLKTVSRWLESATK |
| | | TWGAPERVIIEHVREGFVTEKRAREMDGDMRRRAARNAKLFQEMQEKLNVQGKPSRADLWRYQSVQRQNCQCAYCGSPITFSNSEMDHIVPRAGQGS |
| | | TNTRENLVAVCHRCNQSKGNTPFAIWAKNTSIEGVSVKEAVERTRHWVTDTGMRSTDFKKFTKAVVERFQRATMDEEIDARSMESVAWMANELRSRV |
| | | AQHFASHGTTVRVYRGSLTAEARRASGISGKLEFLDGVGKSRLDRRHHAIDAAVIAFTSDYVAETLAVRSNLKQSQAHRQEAPQWREFTGKDAEHRA |
| | | AWRVWCQKMEKLSALLTEDLRDDRVVVMSNVRLRLGNGSAHEETIGKLSKVKLGSQLSVSDIDKASSEALWCALTREPDFDPKDGLPANPERHIRVN |
| | | GTHVYAGDNIGLFPVSAGSIALRGGYA |
| | | ELGSSFHHARVYKITSGKKPAFAMLRVYTIDLLPYRNQDLFSVELKPQTMSMRQAEKKLRDALATGNAEYLGWLVVDDELVVDTSKIATDQVKAVEA |
| | | ELGTIRRWRVDGFFGDTRLRLRPLQMSKEGIKKESAPELSKIIDRPGWLPAVNKLFSEGNVTVVRRDSLGRVRLESTAHLPVTWKVQPKKKRKV |
| St1Cas9 | 304 | MSDLVLGLDIGIGSVGVGILNKVTGEIIHKNSRIFPAAQAENNLVRRTNRQGRRLTRRKK |
| amino acid | | HRIVRLNRLFEESGLITDFTKISINLNPYQLRVKGLTDELSNEELFIALKNMVKHRGISY |
| sequence | | LDDASDDGNSSVGDYAQIVKENSKQLETKTPGQIQLERYQTYGQLRGDFTVEKDGKKHRL |
| | | INVFPTSAYRSEALRILQTQQEFNPQITDEFINRYLEILTGKRKYYHGPGNEKSRTDYGR |
| | | YRTSGETLDNIFGILIGKCTFYPDEFRAAKASYTAQEFNLLNDLNNLTVPTETKKLSKEQ |
| | | KNQIINYVKNEKAMGPAKLFKYIAKLLSCDVADIKEYRIDKSGKAEIHTFEAYRKMKTLE |
| | | TLDIEQMDRETLDKLAYVLTLNTEREGIQEALEHEFADGSFSQKQVDELVQFRKANSSIF |
| | | GKGWHNFSVKLMMELIPELYETSEEQMTILTRLGKQKTTSSSNKTKYIDEKLLTEEIYNP |
| | | VVAKSVRQAIKIVNAAIKEYGDFDNIVIEMARETNEDDEKKAIQKIQKANKDEKDAAMLK |
| | | AANQYNGKAELPHSVFHGHKQLATKIRLWHQQGERCLYTGKTISIHDLINNSNQFEVDHI |
| | | LPLSITFDDSLANKVLVYATANQEKGQRTPYQALDSMDDAWSFRELKAFVRESKTLSNKK |
| | | KEYLLTEEDISKFDVRKKFIERNLVDTRYASRVVLNALQEHFRAHKIDTKVSVVRGQFTS |
| | | QLRRHWGIEKTRDTYHHHAVDALIIAASIQLNLWKKQKNTLVSYSEDQLLDIETGELISD |
| | | DEYKESVFKAPYQHFVDTLKSKEFEDSILFSYQVDSKFNRKISDATIYATRQAKVGKDKA |
| | | DETYVLGKIKDIYTQDGYDAFMKIYKKDKSKFLMYRHDPQTFEKVIEPILENYPNKQINE |
| | | KGKEVPVNPFLKYKEEHGYIRKYSKKGNGPEIKSLKYYDSKLGNHIDITPKDSNNKVVLQ |
| | | SVSPWRADVYFNKTTGKYEILGLKYADLQFEKGTGTYKISQEKYNDIKKKEGVDSDSEFK |
| | | FTLYKNDLLLVKDTETKEQQLFRFLSRTMPKQKHYVELKPYDKQKFEGGEALIKVLGNVA |
| | | NSGQCKKGLGKSNISIYKVRTDVLGNQHIIKNEGDKPKLDF |
| | 305 | Not used |
| AceCas9 | 306 | MSLQLIIKGVEYGRGADPTMGGSEVGTVPVTWRLGVDVGERSIGLAAVSYEEDKPKEILA |
| amino acid | | AVSWIHDGGVGDERSGASRLALRGMARRARRLRRFRRARLRDLDMLLSELGWTPLPDKNV |
| sequence | | SPVDAWLARKRLAEEYVVDETERRRLLGYAVSHMARHRGWRNPWTTIKDLKNLPQPSDSW |
| | | ERTRESLEARYSVSLEPGTVGQWAGYLLQRAPGIRLNPTQQSAGRRAELSNATAFETRLR |
| | | QEDVLWELRCIADVQGLPEDVVSNVIDAVFCQKRPSVPAERIGRDPLDPSQLRASRACLE |
| | | FQEYRIVAAVANLRIRDGSGSRPLSLEERNAVIEALLAQTERSLTWSDIALEILKLPNES |
| | | DLTSVPEEDGPSSLAYSQFAPFDETSARIAEFIAKNRRKIPTFAQWWQEQDRTSRSDLVA |
| | | ADNSIAGEEEQELLVHLPDAELEALEGLALPSGRVAYSRLTLSGLTRVMRDDGVDVHNAR |
| | | KTCFGVDDNWRPPLPALHEATGHPVVDRNLAILRKFLSSATMRWGPPQSIVVELARGASE |
| | | SRERQAEEEAARRAHRKANDRIRAELRASGLSDPSPADLVRARLLELYDCHCMYCGAPIS |
| | | WENSELDHIVPRTDGGSNRHENLAITCGACNKEKGRRPFASWAETSNRVQLRDVIDRVQK |
| | | LKYSGNMYWTRDEFSRYKKSVVARLKRRTSDPEVIQSIESTGYAAVALRDRLLSYGEKNG |
| | | VAQVAVFRGGVTAEARRWLDISIERLFSRVAIFAQSTSTKRLDRRHHAVDAVVLTTLTPG |
| | | VAKTLADARSRRVSAEFWRRPSDVNRHSTEEPQSPAYRQWKESCSGLGDLLISTAARDSI |
| | | AVAAPLRLRPTGALHEETLRAFSEHTVGAAWKGAELRRIVEPEVYAAFLALTDPGGRFLK |
| | | VSPSEDVLPADENRHIVLSDRVLGPRDRVKLFPDDRGSIRVRGGAAYIASFHHARVFRWG |
| | | SSHSPSFALLRVSLADLAVAGLLRDGVDVFTAELPPWTPAWRYASIALVKAVESGDAKQV |
| | | GWLVPGDELDFGPEGVTTAAGDLSMFLKYFPERHWVVTGFEDDKRINLKPAFLSAEQAEV |
| | | LRTERSDRPDTLTEAGEILAQFFPRCWRATVAKVLCHPGLTVIRRTALGQPRWRRGHLPY |
| | | SWRPWSADPWSGGTP |
| CjeCas9 | 307 | MARILAFDIGISSIGWAFSENDELKDCGVRIFTKAENPKTGESLALPRRLARSARKRLAR |
| amino acid | | RKARLNHLKHLIANEFKLNYEDYQSFDESLAKAYKGSLISPYELRFRALNELLSKQDFAR |
| sequence | | VILHIAKRRGYDDIKNSDDKEKGAILKAIKQNEEKLANYQSVGEYLYKEYFQKFKENSKE |
| | | FTNVRNKKESYERCIAQSFLKDELKLIFKKQREFGFSFSKKFEEEVLSVAFYKRALKDFS |
| | | HLVGNCSFFTDEKRAPKNSPLAFMFVALTRIINLLNNLKNTEGILYTKDDLNALLNEVLK |
| | | NGTLTYKQTKKLLGLSDDYEFKGEKGTYFIEFKKYKEFIKALGDHSLSQDDLNEIAKDIT |
| | | LIKDEIKLKKALAKYDLNQNQIDSLSKLEFKDHLNISFKALKLITPLMLEGKKYDEACNE |
| | | LNLKVAINEDKKDFLPAFNETYYKDEVTNPVVLRAIKEYRKVLNALLKKYGKVHKINIEL |
| | | AREVGKNYSQRAKIEKEQNENYKAKKDAELECEKLGLKINSKNILKLRLFKEQKEFCAYS |
| | | GEKIKISDLQDEKMLEIDHIYPYSRSFDDSYMNKVLVFTKQNQEKLNQTPFEAFGNDSAK |
| | | WQKIEVLAKNLPTKKQKRILDKNYKDKEQKDFKDRNLNDTRYIARLVLNYTKDYLDFLPL |
| | | SDDENTKLNDTQKGSKVHVEAKSGMLTSALRHTWGFSAKDRNNHLHHAIDAVIIAYANNS |
| | | IVKAFSDFKKEQESNSAELYAKKISELDYKNKRKFFEPFSGFRQKVLDKIDEIFVSKPER |
| | | KKPSGALHEETFRKEEEFYQSYGGKEGVLKALELGKIRKVNGKIVKNGDMFRVDIFKHKK |
| | | TNKFYAVPIYTMDFALKVLPNKAVARSKKGEIKDWILMDENYEFCFSLYKDSLILIQTKD |
| | | MQEPEFVYYNAFTSSTVSLIVSKHDNKFETLSKNQKILFKNANEKEVIAKSIGIQNLKVF |
| | | EKYIVSALGEVTKAEFRQREDFKK |
| FnoCas9 | 308 | MNFKILPIAIDLGVKNTGVFSAFYQKGTSLERLDNKNGKVYELSKDSYTLLMNNRTARRH |
| amino acid | | QRRGIDRKQLVKRLFKLIWTEQLNLEWDKDTQQAISFLFNRRGFSFITDGYSPEYLNIVP |
| sequence | | EQVKAILMDIFDDYNGEDDLDSYLKLATEQESKISEIYNKLMQKILEFKLMKLCTDIKDD |
| | | KVSTKTLKEITSYEFELLADYLANYSESLKTQKFSYTDKQGNLKELSYYHHDKYNIQEFL |
| | | KRHATINDRILDTLLTDDLDIWNFNFEKFDFDKNEEKLQNQEDKDHIQAHLHHFVFAVNK |
| | | IKSEMASGGRHRSQYFQEITNVLDENNHQEGYLKNFCENLHNKKYSNLSVKNLVNLIGNL |
| | | SNLELKPLRKYFNDKIHAKADHWDEQKFTETYCHWILGEWRVGVKDQDKKDGAKYSYKDL |
| | | CNELKQKVTKAGLVDFLLELDPCRTIPPYLDNNNRKPPKCQSLILNPKFLDNQYPNWQQY |
| | | LQELKKLQSIQNYLDSFETDLKVLKSSKDQPYFVEYKSSNQQIASGQRDYKDLDARILQF |
| | | IFDRVKASDELLLNEIYFQAKKLKQKASSELEKLESSKKLDEVIANSQLSQILKSQHTNG |
| | | IFEQGTFLHLVCKYYKQRQRARDSRLYIMPEYRYDKKLHKYNNTGRFDDDNQLLTYCNHK |
| | | PRQKRYQLLNDLAGVLQVSPNFLKDKIGSDDDLFISKWLVEHIRGFKKACEDSLKIQKDN |
| | | RGLLNHKINIARNTKGKCEKEIFNLICKIEGSEDKKGNYKHGLAYELGVLLFGEPNEASK |
| | | PEFDRKIKKFNSIYSFAQIQQIAFAERKGNANTCAVCSADNAHRMQQIKITEPVEDNKDK |
| | | IILSAKAQRLPAIPTRIVDGAVKKMATILAKNIVDDNWQNIKQVLSAKHQLHIPIITESN |
| | | AFEFEPALADVKGKSLKDRRKKALERISPENIFKDKNNRIKEFAKGISAYSGANLTDGDF |
| | | DGAKEELDHIIPRSHKKYGTLNDEANLICVTRGDNKNKGNRIFCLRDLADNYKLKQFETT |
| | | DDLEIEKKIADTIWDANKKDFKFGNYRSFINLTPQEQKAFRHALFLADENPIKQAVIRAI |
| | | NNRNRTFVNGTQRYFAEVLANNIYLRAKKENLNTDKISFDYFGIPTIGNGRGIAEIRQLY |
| | | EKVDSDIQAYAKGDKPQASYSHLIDAMLAFCIAADEHRNDGSIGLEIDKNYSLYPLDKNT |
| | | GEVFTKDIFSQIKITDNEFSDKKLVRKKAIEGFNTHRQMTRDGIYAENYLPILIHKELNE |
| | | VRKGYTWKNSEEIKIFKGKKYDIQQLNNLVYCLKFVDKPISIDIQISTLEELRNILTTNN |
| | | IAATAEYYYINLKTQKLHEYYIENYNTALGYKKYSKEMEFLRSLAYRSERVKIKSIDDVK |
| | | QVLDKDSNFIIGKITLPFKKEWQRLYREWQNTTIKDDYEFLKSFFNVKSITKLHKKVRKD |
| | | FSLPISTNEGKFLVKRKTWDNNFIYQILNDSDSRVDGTKPFIPAFDISKNEIVEAIIDSF |
| | | TSKNIFWLPKNIELQKVDNKNIFAIDTSKWFEVETPSDLRDIGVATIQYKIDNNSRPKVR |
| | | VKLDYVIDDDSKINYFTNHSLLKSRYPDKVLEILKQSTTIEFESSGFNKTIKEMLGMTLA |
| | | GIYNETSNN |
| AsCpf1 | 309 | MTQFEGFTNLYQVSKTLRFELIPQGKTLKHIQEQGFIEEDKARNDHYKELKPIIDRIYKT |
| amino acid | | YADQCLQLVQLDWENLSAAIDSYRKEKTEETRNALIEEQATYRNAIHDYFIGRTDNLTDA |
| sequence | | INKRHAEIYKGLFKAELFNGKVLKQLGTVTTTEHENALLRSFDKFTTYFSGFYENRKNVF |
| | | SAEDISTAIPHRIVQDNFPKFKENCHIFTRLITAVPSLREHFENVKKAIGIFVSTSIEEV |
| | | FSFPFYNQLLTQTQIDLYNQLLGGISREAGTEKIKGLNEVLNLAIQKNDETAHIIASLPH |
| | | RFIPLFKQILSDRNTLSFILEEFKSDEEVIQSFCKYKTLLRNENVLETAEALFNELNSID |
| | | LTHIFISHKKLETISSALCDHWDTLRNALYERRISELTGKITKSAKEKVQRSLKHEDINL |
| | | QEIISAAGKELSEAFKQKTSEILSHAHAALDQPLPTTLKKQEEKEILKSQLDSLLGLYHL |
| | | LDWFAVDESNEVDPEFSARLTGIKLEMEPSLSFYNKARNYATKKPYSVEKFKLNFQMPTL |
| | | ASGWDVNKEKNNGAILFVKNGLYYLGIMPKQKGRYKALSFEPTEKTSEGEDKMYYDYFPD |
| | | AAKMIPKCSTQLKAVTAHFQTHTTPILLSNNFIEPLEITKEIYDLNNPEKEPKKFQTAYA |
| | | KKTGDQKGYREALCKWIDFTRDELSKYTKTTSIDLSSLRPSSQYKDLGEYYAELNPLLYH |
| | | ISFQRIAEKEIMDAVETGKLYLFQIYNKDFAKGHHGKPNLHTLYWTGLESPENLAKTSIK |
| | | LNGQAELFYRPKSRMKRMAHRLGEKMLNKKLKDQKTPIPDTLYQELYDYVNHRLSHDLSD |
| | | EARALLPNVITKEVSHEIIKDRRFTSDKFFFHVPITLNYQAANSPSKENQRVNAYLKEHP |
| | | ETPIIGIDRGERNLIYITVIDSTGKILEQRSLNTIQQFDYQKKLDNREKERVAARQAWSV |
| | | VGTIKDLKQGYLSQVIHEIVDLMIHYQAVVVLENLNFGFKSKRTGIAEKAVYQQFEKMLI |
| | | DKLNCLVLKDYPAEKVGGVLNPYQLTDQFTSFAKMGTQSGELFYVPAPYTSKIDPLTGFV |
| | | DPFVWKTIKNHESRKHFLEGFDFLHYDVKTGDFILHEKMNRNLSFQRGLPGFMPAWDIVF |
| | | EKNETQFDAKGTPFIAGKRIVPVIENHRFTGRYRDLYPANELIALLEEKGIVFRDGSNIL |
| | | PKLLENDDSHAIDTMVALIRSVLQMRNSNAATGEDYINSPVRDLNGVCFDSRFQNPEWPM |
| | | DADANGAYHIALKGQLLLNHLKESKDLKLQNGISNQDWLAYIQELRN |
| EsCas13d | 310 | MAAFKPNPINYILGLDIGIASVGWAMVEIDEEENPIRLIDLGVRVFERAEVPKTGDSLAM |
| amino acid | | ARRLARSVRRLTRRRAHRLLRARRLLKREGVLQAADFDENGLIKSLPNTPWQLRAAALDR |
| sequence | | KLTPLEWSAVLLHLIKHRGYLSQRKNEGETADKELGALLKGVANNAHALQTGDERTPAEL |
| | | ALNKFEKESGHIRNQRGDYSHTFSRKDLQAELILLFEKQKEFGNPHVSGGLKEGIETLLM |
| | | TQRPALSGDAVQKMLGHCTFEPAEPKAAKNTYTAERFIWLTKLNNLRILEQGSERPLTDT |
| | | ERATLMDEPYRKSKLTYAQARKLLGLEDTAFFKGLRYGKDNAEASTLMEMKAYHAISRAL |
| | | EKEGLKDKKSPLNLSSELQDEIGTAFSLEKTDEDITGRLKDRVQPEILEALLKHISFDKF |
| | | VQISLKALRRIVPLMEQGKRYDEACAEIYGDHYGKKNTEEKIYLPPIPADEIRNPVVLRA |
| | | LSQARKVINGVVRRYGSPARIHIETAREVGKSFKDRKEIEKRQEENRKDREKAAAKFREY |
| | | FPNFVGEPKSKDILKLRLYEQQHGKCLYSGKEINLVRLNEKGYVEIDHALPFSRTWDDSF |
| | | NNKVLVLGSENQNKGNQTPYEYENGKDNSREWQEFKARVETSRFPRSKKQRILLQKFDED |
| | | GFKECNLNDTRYVNRFLCQFVADHILLTGKGKRRVFASNGQITNLLRGFWGLRKVRAEND |
| | | RHHALDAVVVACSTVAMQQKITRFVRYKEMNAFDGKTIDKETGKVLHQKTHFPQPWEFFA |
| | | QEVMIRVFGKPDGKPEFEEADTPEKLRTLLAEKLSSRPEAVHEYVTPLFVSRAPNRKMSG |
| | | AHKDTLRSAKRFVKHNEKISVKRVWLTEIKLADLENMVNYKNGREIELYEALKARLEAYG |
| | | GNAKQAFDPKDNPFYKKGGQLVKAVRVEKTQESGVLLNKKNAYTIADNGDMVRVDVFCKV |
| | | DKKGKNQYFIVPIYAWQVAENILPDIDCKGYRIDDSYTFCFSLHKYDLIAFQKDEKSKVE |
| | | FAYYINCDSSNGRFYLAWHDKGSKEQQFRISTQNLVLIQKYQVNELGKEIRPCRLKKRPP |
| | | VR |
| RruCas9 | 311 | MRPIEPWILGLDIGTDSLGWAVFSCEEKGPPTAKELLGGGVRLFDSGRDAKDHTSRQAER |
| amino acid | | GAFRRARRQTRTWPWRRDRLIALFQAAGLTPPAAETRQIALALRREAVSRPLAPDALWAA |
| sequence | | LLHLAHHRGFRSNRIDKRERAAAKALAKAKPAKATAKATAPAKEADDEAGFWEGAEAALR |
| | | QRMAASGAPTVGALLADDLDRGQPVRMRYNQSDRDGVVAPTRALIAEELAEIVARQSSAY |
| | | PGLDWPAVTRLVLDQRPLRSKGAGPCAFLPGEDRALRALPTVQDFIIRQTLANLRLPSTS |
| | | ADEPRPLTDEEHAKALALLSTARFVEWPALRRALGLKRGVKFTAETERNGAKQAARGTAG |
| | | NLTEAILAPLIPGWSGWDLDRKDRVFSDLWAARQDRSALLALIGDPRGPTRVTEDETAEA |
| | | VADAIQIVLPTGRASLSAKAARAIAQAMAPGIGYDEAVTLALGLHHSHRPRQERLARLPY |
| | | YAAALPDVGLDGDPVGPPPAEDDGAAAEAYYGRIGNISVHIALNETRKIVNALLHRHGPI |
| | | LRLVMVETTRELKAGADERKRMIAEQAERERENAEIDVELRKSDRWMANARERRQRVRLA |
| | | RRQNNLCPYTSTPIGHADLLGDAYDIDHVIPLARGGRDSLDNMVLCQSDANKTKGDKTPW |
| | | EAFHDKPGWIAQRDDFLARLDPQTAKALAWRFADDAGERVARKSAEDEDQGELPRQLTDT |
| | | GYIARVALRYLSLVTNEPNAVVATNGRLTGLLRLAWDITPGPAPRDLLPTPRDALRDDTA |
| | | ARRFLDGLTPPPLAKAVEGAVQARLAALGRSRVADAGLADALGLTLASLGGGGKNRADHR |
| | | HHFIDAAMIAVTTRGLINQINQASGAGRILDLRKWPRTNFEPPYPTFRAEVMKQWDHIHP |
| | | SIRPAHRDGGSLHAATVFGVRNRPDARVLVQRKPVEKLFLDANAKPLPADKIAEIIDGFA |
| | | SPRMAKRFKALLARYQAAHPEVPPALAALAVARDPAFGPRGMTANTVIAGRSDGDGEDAG |
| | | LITPFRANPKAAVRTMGNAVYEVWEIQVKGRPRWTHRVLTRFDRTQPAPPPPPENARLVM |
| | | RLRRGDLVYWPLESGDRLFLVKKMAVDGRLALWPARLATGKATALYAQLSCPNINLNGDQ |
| | | GYCVQSAEGIRKEKIRTTSCTALGRLRLSKKAT |
| RpaCas9 | 312 | MLERHPQRYRLGLDLGSNSLGWFVTNLQQRGDRFEPVGLGPGGVRIFPDGRDPQSKASNA |
| amino acid | | VDRRMARGARKRRDRFVQRRSQLMDALVRHGLMPEEAAARKALAGLDPYQLRARALVDRL |
| sequence | | PAHHVGRALFHLNQRRGELSNRKTDNKKNSEDGAIKQAASRLRDSMASQGAETLGEFFAG |
| | | RRHSDTYAERQTAIRAELQRIGKDHLTGNARKKAWAKVRKRLFGDDVLDPAMAPEGVRAR |
| | | AIITGTKASYDFYPTRDLLLQEFHAIWRAQAPHHSTMTESACREIERIIFYQRPLKDPIV |
| | | GKCSLDPATRPFKEDPEGYRAPWAHPLAQRFRILSEARNLEIRETGRGSRKLTKAQSDVV |
| | | ALALLGSKEVKFDKLRTLLKLPAESKENLESDRRAALDGDATAARLSDKNGFGKAWRGFP |
| | | LERQVAIVERLMTVEVEAELVDWLEQECGLGPEAAARVANTSLPEGHCRLGLRAIKKIVP |
| | | VMQNEVGDDGVSGAGYYEAAKRIGYDHAKLPTGGDLDYLPYYGKWLADAVVGSGDARDGK |
| | | ERQFGQFPNPTVHIGLGQLRRLVNELIRAYGPPTEISIEFTRALRLSEDQKAQVQREQRK |
| | | NQDRNRARAAELEQLGFPANPRNLLKMRLWEELNLRDPLDRKCVYTGEQISIERLLSEEV |
| | | DIDHILPVAMTLDDSAANKIVCMRYANRHKRKQTPFEAFGSSPLVQGRRYAWDDIATRAA |
| | | ALPRNKRWRFDADAREQFDKRGGFLARQLNETGWLARLAKEYLGAVTDPNRIWVVPGRLT |
| | | GLLRGKWGLNALLPDHNYAGVQDKAEAFLASTDDMEFSGVKNRADHRHHAIDGLVAALTD |
| | | RSLLWKMANAYDDEREKFVVELPWPTMRDDLKAALDKMVVSHKPDHGVQGKLHEDSAYGL |
| | | LDRPEPVEDDDREPANLVYRKAVETLSENEIGRIRDRRLRDLVRDHVDAAKRNGVALADA |
| | | LRQLTEPSDNPHFRHGLRHVRLLKKEKTDYLVPVVDRATGRPYKAYSAGENFCVEVFETA |
| | | DGKWSGEAVRREDANRSNCGPKTPHVPRWRTESAGARLVMRIHKGDLIRLEHEGRTRIMV |
| | | VHRLDAAAGREKLAAHNETGNLDKRHATDNDIDPFRWLMASYGTLKKMAAVPVRVDELGR |
| | | VWRIDPR |
| AnaCas9 | 313 | MWYASLMSAHHLRVGIDVGTHSVGLATLRVDDHGTPIELLSALSHIHDSGVGKEGKKDHD |
| amino acid | | TRKKLSGIARRARRLLHHRRTQLQQLDEVLRDLGFPIPTPGEFLDLNEQTDPYRVWRVRA |
| sequence | | RLVEEKLPEELRGPAISMAVRHIARHRGWRNPYSKVESLLSPAEESPEMKALRERILATT |
| | | GEVLDDGITPGQAMAQVALTHNISMRGPEGILGKLHQSDNANEIRKICARQGVSPDVCKQ |
| | | LLRAVEKADSPRGSAVSRVAPDPLPGQGSFRRAPKCDPEFQRFRIISIVANLRISETKGE |
| | | NRPLTADERRHVVTFLTEDSQADLTWVDVAEKLGVHRRDLRGTAVHTDDGERSAARPPID |
| | | ATDRIMRQTKISSLKTWWEEADSEQRGAMIRYLYEDPTDSECAEIIAELPEEDQAKLDSL |
| | | HLPAGRAAYSRESLTALSDHMLATTDDLHEARKRLFGVDDSWAPPAEAINAPVGNPSVDR |
| | | TLKIVGRYLSAVESMWGTPEVIHVEHVRDGFTSERMADERDKANRRRYNDNQEAMKKIQR |
| | | DYGKEGYISRGDIVRLDALELQGCACLYCGTTIGYHTCQLDHIVPQAGPGSNNRRGNLVA |
| | | VCERCNRSKSNTPFAVWAQKCGIPHVGVKEAIGRVRGWRKQTPNTSSEDLTRLKKEVIAR |
| | | LRRTQEDPEIDERSMESVAWMANELHHRIAAAYPETTVMVYRGSITAAARKAAGIDSRIN |
| | | LIGEKGRKDRIDRRHHAVDASVVALMEASVAKTLAERSSLRGEQRLTGKEQTWKQYTGST |
| | | VGAREHFEMWRGHMLHLTELFNERLAEDKVYVTQNIRLRLSDGNAHTVNPSKLVSHRLGD |
| | | GLTVQQIDRACTPALWCALTREKDFDEKNGLPAREDRAIRVHGHEIKSSDYIQVESKRKK |
| | | TDSDRDETPFGAIAVRGGFVEIGPSIHHARIYRVEGKKPVYAMLRVETHDLLSQRHGDLE |
| | | SAVIPPQSISMRCAEPKLRKAITTGNATYLGWVVVGDELEINVDSFTKYAIGRFLEDFPN |
| | | TTRWRICGYDTNSKLTLKPIVLAAEGLENPSSAVNEIVELKGWRVAINVLTKVHPTVVRR |
| | | DALGRPRYSSRSNLPTSWTIE |
| | 316-320 | Not used |
| SpyCas9 | 321 | AUGGACAAGAAGUACAGCAUCGGACUGGACAUCGGAACAAACAGCGUCGGAUGGGCAGUCAUCACAGACGAAUACAAGGUCCCGAGCAAGAAGUUCA |
| mRNA | | AGGUCCUGGGAAACACAGACAGACACAGCAUCAAGAAGAACCUGAUCGGAGCACUGCUGUUCGACAGCGGAGAAACAGCAGAAGCAACAAGACUGAA |
| sequence | | GAGAACAGCAAGAAGAAGAUACACAAGAAGAAAGAACAGAAUCUGCUACCUGCAGGAAAUCUUCAGCAACGAAAUGGCAAAGGUCGACGACAGCUUC |
| with ORF | | UUCCACAGACUGGAAGAAAGCUUCCUGGUCGAAGAAGACAAGAAGCACGAAAGACACCCGAUCUUCGGAAACAUCGUCGACGAAGUCGCAUACCACG |
| | | AAAAGUACCCGACAAUCUACCACCUGAGAAAGAAGCUGGUCGACAGCACAGACAAGGCAGACCUGAGACUGAUCUACCUGGCACUGGCACACAUGAU |
| | | CAAGUUCAGAGGACACUUCCUGAUCGAAGGAGACCUGAACCCGGACAACAGCGACGUCGACAAGCUGUUCAUCCAGCUGGUCCAGACAUACAACCAG |
| | | CUGUUCGAAGAAAACCCGAUCAACGCAAGCGGAGUCGACGCAAAGGCAAUCCUGAGCGCAAGACUGAGCAAGAGCAGAAGACUGGAAAACCUGAUCG |
| | | CACAGCUGCCGGGAGAAAAGAAGAACGGACUGUUCGGAAACCUGAUCGCACUGAGCCUGGGACUGACACCGAACUUCAAGAGCAACUUCGACCUGGC |
| | | AGAAGACGCAAAGCUGCAGCUGAGCAAGGACACAUACGACGACGACCUGGACAACCUGCUGGCACAGAUCGGAGACCAGUACGCAGACCUGUUCCUG |
| | | GCAGCAAAGAACCUGAGCGACGCAAUCCUGCUGAGCGACAUCCUGAGAGUCAACACAGAAAUCACAAAGGCACCGCUGAGCGCAAGCAUGAUCAAGA |
| | | GAUACGACGAACACCACCAGGACCUGACACUGCUGAAGGCACUGGUCAGACAGCAGCUGCCGGAAAAGUACAAGGAAAUCUUCUUCGACCAGAGCAA |
| | | GAACGGAUACGCAGGAUACAUCGACGGAGGAGCAAGCCAGGAAGAAUUCUACAAGUUCAUCAAGCCGAUCCUGGAAAAGAUGGACGGAACAGAAGAA |
| | | CUGCUGGUCAAGCUGAACAGAGAAGACCUGCUGAGAAAGCAGAGAACAUUCGACAACGGAAGCAUCCCGCACCAGAUCCACCUGGGAGAACUGCACG |
| | | CAAUCCUGAGAAGACAGGAAGACUUCUACCCGUUCCUGAAGGACAACAGAGAAAAGAUCGAAAAGAUCCUGACAUUCAGAAUCCCGUACUACGUCGG |
| | | ACCGCUGGCAAGAGGAAACAGCAGAUUCGCAUGGAUGACAAGAAAGAGCGAAGAAACAAUCACACCGUGGAACUUCGAAGAAGUCGUCGACAAGGGA |
| | | GCAAGCGCACAGAGCUUCAUCGAAAGAAUGACAAACUUCGACAAGAACCUGCCGAACGAAAAGGUCCUGCCGAAGCACAGCCUGCUGUACGAAUACU |
| | | UCACAGUCUACAACGAACUGACAAAGGUCAAGUACGUCACAGAAGGAAUGAGAAAGCCGGCAUUCCUGAGCGGAGAACAGAAGAAGGCAAUCGUCGA |
| | | CCUGCUGUUCAAGACAAACAGAAAGGUCACAGUCAAGCAGCUGAAGGAAGACUACUUCAAGAAGAUCGAAUGCUUCGACAGCGUCGAAAUCAGCGGA |
| | | GUCGAAGACAGAUUCAACGCAAGCCUGGGAACAUACCACGACCUGCUGAAGAUCAUCAAGGACAAGGACUUCCUGGACAACGAAGAAAACGAAGACA |
| | | UCCUGGAAGACAUCGUCCUGACACUGACACUGUUCGAAGACAGAGAAAUGAUCGAAGAAAGACUGAAGACAUACGCACACCUGUUCGACGACAAGGU |
| | | CAUGAAGCAGCUGAAGAGAAGAAGAUACACAGGAUGGGGAAGACUGAGCAGAAAGCUGAUCAACGGAAUCAGAGACAAGCAGAGCGGAAAGACAAUC |
| | | CUGGACUUCCUGAAGAGCGACGGAUUCGCAAACAGAAACUUCAUGCAGCUGAUCCACGACGACAGCCUGACAUUCAAGGAAGACAUCCAGAAGGCAC |
| | | AGGUCAGCGGACAGGGAGACAGCCUGCACGAACACAUCGCAAACCUGGCAGGAAGCCCGGCAAUCAAGAAGGGAAUCCUGCAGACAGUCAAGGUCGU |
| | | CGACGAACUGGUCAAGGUCAUGGGAAGACACAAGCCGGAAAACAUCGUCAUCGAAAUGGCAAGAGAAAACCAGACAACACAGAAGGGACAGAAGAAC |
| | | AGCAGAGAAAGAAUGAAGAGAAUCGAAGAAGGAAUCAAGGAACUGGGAAGCCAGAUCCUGAAGGAACACCCGGUCGAAAACACACAGCUGCAGAACG |
| | | AAAAGCUGUACCUGUACUACCUGCAGAACGGAAGAGACAUGUACGUCGACCAGGAACUGGACAUCAACAGACUGAGCGACUACGACGUCGACCACAU |
| | | CGUCCCGCAGAGCUUCCUGAAGGACGACAGCAUCGACAACAAGGUCCUGACAAGAAGCGACAAGAACAGAGGAAAGAGCGACAACGUCCCGAGCGAA |
| | | GAAGUCGUCAAGAAGAUGAAGAACUACUGGAGACAGCUGCUGAACGCAAAGCUGAUCACACAGAGAAAGUUCGACAACCUGACAAAGGCAGAGAGAG |
| | | GAGGACUGAGCGAACUGGACAAGGCAGGAUUCAUCAAGAGACAGCUGGUCGAAACAAGACAGAUCACAAAGCACGUCGCACAGAUCCUGGACAGCAG |
| | | AAUGAACACAAAGUACGACGAAAACGACAAGCUGAUCAGAGAAGUCAAGGUCAUCACACUGAAGAGCAAGCUGGUCAGCGACUUCAGAAAGGACUUC |
| | | CAGUUCUACAAGGUCAGAGAAAUCAACAACUACCACCACGCACACGACGCAUACCUGAACGCAGUCGUCGGAACAGCACUGAUCAAGAAGUACCCGA |
| | | AGCUGGAAAGCGAAUUCGUCUACGGAGACUACAAGGUCUACGACGUCAGAAAGAUGAUCGCAAAGAGCGAACAGGAAAUCGGAAAGGCAACAGCAAA |
| | | GUACUUCUUCUACAGCAACAUCAUGAACUUCUUCAAGACAGAAAUCACACUGGCAAACGGAGAAAUCAGAAAGAGACCGCUGAUCGAAACAAACGGA |
| | | GAAACAGGAGAAAUCGUCUGGGACAAGGGAAGAGACUUCGCAACAGUCAGAAAGGUCCUGAGCAUGCCGCAGGUCAACAUCGUCAAGAAGACAGAAG |
| | | UCCAGACAGGAGGAUUCAGCAAGGAAAGCAUCCUGCCGAAGAGAAACAGCGACAAGCUGAUCGCAAGAAAGAAGGACUGGGACCCGAAGAAGUACGG |
| | | AGGAUUCGACAGCCCGACAGUCGCAUACAGCGUCCUGGUCGUCGCAAAGGUCGAAAAGGGAAAGAGCAAGAAGCUGAAGAGCGUCAAGGAACUGCUG |
| | | GGAAUCACAAUCAUGGAAAGAAGCAGCUUCGAAAAGAACCCGAUCGACUUCCUGGAAGCAAAGGGAUACAAGGAAGUCAAGAAGGACCUGAUCAUCA |
| | | AGCUGCCGAAGUACAGCCUGUUCGAACUGGAAAACGGAAGAAAGAGAAUGCUGGCAAGCGCAGGAGAACUGCAGAAGGGAAACGAACUGGCACUGCC |
| | | GAGCAAGUACGUCAACUUCCUGUACCUGGCAAGCCACUACGAAAAGCUGAAGGGAAGCCCGGAAGACAACGAACAGAAGCAGCUGUUCGUCGAACAG |
| | | CACAAGCACUACCUGGACGAAAUCAUCGAACAGAUCAGCGAAUUCAGCAAGAGAGUCAUCCUGGCAGACGCAAACCUGGACAAGGUCCUGAGCGCAU |
| | | ACAACAAGCACAGAGACAAGCCGAUCAGAGAACAGGCAGAAAACAUCAUCCACCUGUUCACACUGACAAACCUGGGAGCACCGGCAGCAUUCAAGUA |
| | | CUUCGACACAACAAUCGACAGAAAGAGAUACACAAGCACAAAGGAAGUCCUGGACGCAACACUGAUCCACCAGAGCAUCACAGGACUGUACGAAACA |
| | | AGAAUCGACCUGAGCCAGCUGGGAGGAGACGGAGGAGGAAGCCCGAAGAAGAAGAGAAAGGUCUAG |
| SpyCas9 | 322 | AUGGACAAGAAGUACUCCAUCGGCCUGGACAUCGGCACCAACUCCGUGGGCUGGGCCGUGAUCACCGACGAGUACAAGGUGCCCUCCAAGAAGUUCA |
| mRNA | | AGGUGCUGGGCAACACCGACCGGCACUCCAUCAAGAAGAACCUGAUCGGCGCCCUGCUGUUCGACUCCGGCGAGACCGCCGAGGCCACCCGGCUGAA |
| sequence | | GCGGACCGCCCGGCGGCGGUACACCCGGCGGAAGAACCGGAUCUGCUACCUGCAGGAGAUCUUCUCCAACGAGAUGGCCAAGGUGGACGACUCCUUC |
| with ORF | | UUCCACCGGCUGGAGGAGUCCUUCCUGGUGGAGGAGGACAAGAAGCACGAGCGGCACCCCAUCUUCGGCAACAUCGUGGACGAGGUGGCCUACCACG |
| | | AGAAGUACCCCACCAUCUACCACCUGCGGAAGAAGCUGGUGGACUCCACCGACAAGGCCGACCUGCGGCUGAUCUACCUGGCCCUGGCCCACAUGAU |
| | | CAAGUUCCGGGGCCACUUCCUGAUCGAGGGCGACCUGAACCCCGACAACUCCGACGUGGACAAGCUGUUCAUCCAGCUGGUGCAGACCUACAACCAG |
| | | CUGUUCGAGGAGAACCCCAUCAACGCCUCCGGCGUGGACGCCAAGGCCAUCCUGUCCGCCCGGCUGUCCAAGUCCCGGCGGCUGGAGAACCUGAUCG |
| | | CCCAGCUGCCCGGCGAGAAGAAGAACGGCCUGUUCGGCAACCUGAUCGCCCUGUCCCUGGGCCUGACCCCCAACUUCAAGUCCAACUUCGACCUGGC |
| | | CGAGGACGCCAAGCUGCAGCUGUCCAAGGACACCUACGACGACGACCUGGACAACCUGCUGGCCCAGAUCGGCGACCAGUACGCCGACCUGUUCCUG |
| | | GCCGCCAAGAACCUGUCCGACGCCAUCCUGCUGUCCGACAUCCUGCGGGUGAACACCGAGAUCACCAAGGCCCCCCUGUCCGCCUCCAUGAUCAAGC |
| | | GGUACGACGAGCACCACCAGGACCUGACCCUGCUGAAGGCCCUGGUGCGGCAGCAGCUGCCCGAGAAGUACAAGGAGAUCUUCUUCGACCAGUCCAA |
| | | GAACGGCUACGCCGGCUACAUCGACGGCGGCGCCUCCCAGGAGGAGUUCUACAAGUUCAUCAAGCCCAUCCUGGAGAAGAUGGACGGCACCGAGGAG |
| | | CUGCUGGUGAAGCUGAACCGGGAGGACCUGCUGCGGAAGCAGCGGACCUUCGACAACGGCUCCAUCCCCCACCAGAUCCACCUGGGCGAGCUGCACG |
| | | CCAUCCUGCGGCGGCAGGAGGACUUCUACCCCUUCCUGAAGGACAACCGGGAGAAGAUCGAGAAGAUCCUGACCUUCCGGAUCCCCUACUACGUGGG |
| | | CCCCCUGGCCCGGGGCAACUCCCGGUUCGCCUGGAUGACCCGGAAGUCCGAGGAGACCAUCACCCCCUGGAACUUCGAGGAGGUGGUGGACAAGGGC |
| | | GCCUCCGCCCAGUCCUUCAUCGAGCGGAUGACCAACUUCGACAAGAACCUGCCCAACGAGAAGGUGCUGCCCAAGCACUCCCUGCUGUACGAGUACU |
| | | UCACCGUGUACAACGAGCUGACCAAGGUGAAGUACGUGACCGAGGGCAUGCGGAAGCCCGCCUUCCUGUCCGGCGAGCAGAAGAAGGCCAUCGUGGA |
| | | CCUGCUGUUCAAGACCAACCGGAAGGUGACCGUGAAGCAGCUGAAGGAGGACUACUUCAAGAAGAUCGAGUGCUUCGACUCCGUGGAGAUCUCCGGC |
| | | GUGGAGGACCGGUUCAACGCCUCCCUGGGCACCUACCACGACCUGCUGAAGAUCAUCAAGGACAAGGACUUCCUGGACAACGAGGAGAACGAGGACA |
| | | UCCUGGAGGACAUCGUGCUGACCCUGACCCUGUUCGAGGACCGGGAGAUGAUCGAGGAGCGGCUGAAGACCUACGCCCACCUGUUCGACGACAAGGU |
| | | GAUGAAGCAGCUGAAGCGGCGGCGGUACACCGGCUGGGGCCGGCUGUCCCGGAAGCUGAUCAACGGCAUCCGGGACAAGCAGUCCGGCAAGACCAUC |
| | | CUGGACUUCCUGAAGUCCGACGGCUUCGCCAACCGGAACUUCAUGCAGCUGAUCCACGACGACUCCCUGACCUUCAAGGAGGACAUCCAGAAGGCCC |
| | | AGGUGUCCGGCCAGGGCGACUCCCUGCACGAGCACAUCGCCAACCUGGCCGGCUCCCCCGCCAUCAAGAAGGGCAUCCUGCAGACCGUGAAGGUGGU |
| | | GGACGAGCUGGUGAAGGUGAUGGGCCGGCACAAGCCCGAGAACAUCGUGAUCGAGAUGGCCCGGGAGAACCAGACCACCCAGAAGGGCCAGAAGAAC |
| | | UCCCGGGAGCGGAUGAAGCGGAUCGAGGAGGGCAUCAAGGAGCUGGGCUCCCAGAUCCUGAAGGAGCACCCCGUGGAGAACACCCAGCUGCAGAACG |
| | | AGAAGCUGUACCUGUACUACCUGCAGAACGGCCGGGACAUGUACGUGGACCAGGAGCUGGACAUCAACCGGCUGUCCGACUACGACGUGGACCACAU |
| | | CGUGCCCCAGUCCUUCCUGAAGGACGACUCCAUCGACAACAAGGUGCUGACCCGGUCCGACAAGAACCGGGGCAAGUCCGACAACGUGCCCUCCGAG |
| | | GAGGUGGUGAAGAAGAUGAAGAACUACUGGCGGCAGCUGCUGAACGCCAAGCUGAUCACCCAGCGGAAGUUCGACAACCUGACCAAGGCCGAGCGGG |
| | | GCGGCCUGUCCGAGCUGGACAAGGCCGGCUUCAUCAAGCGGCAGCUGGUGGAGACCCGGCAGAUCACCAAGCACGUGGCCCAGAUCCUGGACUCCCG |
| | | GAUGAACACCAAGUACGACGAGAACGACAAGCUGAUCCGGGAGGUGAAGGUGAUCACCCUGAAGUCCAAGCUGGUGUCCGACUUCCGGAAGGACUUC |
| | | CAGUUCUACAAGGUGCGGGAGAUCAACAACUACCACCACGCCCACGACGCCUACCUGAACGCCGUGGUGGGCACCGCCCUGAUCAAGAAGUACCCCA |
| | | AGCUGGAGUCCGAGUUCGUGUACGGCGACUACAAGGUGUACGACGUGCGGAAGAUGAUCGCCAAGUCCGAGCAGGAGAUCGGCAAGGCCACCGCCAA |
| | | GUACUUCUUCUACUCCAACAUCAUGAACUUCUUCAAGACCGAGAUCACCCUGGCCAACGGCGAGAUCCGGAAGCGGCCCCUGAUCGAGACCAACGGC |
| | | GAGACCGGCGAGAUCGUGUGGGACAAGGGCCGGGACUUCGCCACCGUGCGGAAGGUGCUGUCCAUGCCCCAGGUGAACAUCGUGAAGAAGACCGAGG |
| | | UGCAGACCGGCGGCUUCUCCAAGGAGUCCAUCCUGCCCAAGCGGAACUCCGACAAGCUGAUCGCCCGGAAGAAGGACUGGGACCCCAAGAAGUACGG |
| | | CGGCUUCGACUCCCCCACCGUGGCCUACUCCGUGCUGGUGGUGGCCAAGGUGGAGAAGGGCAAGUCCAAGAAGCUGAAGUCCGUGAAGGAGCUGCUG |
| | | GGCAUCACCAUCAUGGAGCGGUCCUCCUUCGAGAAGAACCCCAUCGACUUCCUGGAGGCCAAGGGCUACAAGGAGGUGAAGAAGGACCUGAUCAUCA |
| | | AGCUGCCCAAGUACUCCCUGUUCGAGCUGGAGAACGGCCGGAAGCGGAUGCUGGCCUCCGCCGGCGAGCUGCAGAAGGGCAACGAGCUGGCCCUGCC |
| | | CUCCAAGUACGUGAACUUCCUGUACCUGGCCUCCCACUACGAGAAGCUGAAGGGCUCCCCCGAGGACAACGAGCAGAAGCAGCUGUUCGUGGAGCAG |
| | | CACAAGCACUACCUGGACGAGAUCAUCGAGCAGAUCUCCGAGUUCUCCAAGCGGGUGAUCCUGGCCGACGCCAACCUGGACAAGGUGCUGUCCGCCU |
| | | ACAACAAGCACCGGGACAAGCCCAUCCGGGAGCAGGCCGAGAACAUCAUCCACCUGUUCACCCUGACCAACCUGGGCGCCCCCGCCGCCUUCAAGUA |
| | | CUUCGACACCACCAUCGACCGGAAGCGGUACACCUCCACCAAGGAGGUGCUGGACGCCACCCUGAUCCACCAGUCCAUCACCGGCCUGUACGAGACC |
| | | CGGAUCGACCUGUCCCAGCUGGGCGGCGACGGCGGCGGCUCCCCCAAGAAGAAGCGGAAGGUGUGA |
| SpyCas9 | 323 | AUGGACAAGAAGUACUCCAUCGGCCUGGACAUCGGCACCAACUCCGUGGGCUGGGCCGUGAUCACCGACGAGUACAAGGUGCCCUCCAAGAAGUUCA |
| mRNA | | AGGUGCUGGGCAACACCGACCGGCACUCCAUCAAGAAGAACCUGAUCGGCGCCCUGCUGUUCGACUCCGGCGAGACCGCCGAGGCCACCCGGCUGAA |
| sequence | | GCGGACCGCCCGGCGGCGGUACACCCGGCGGAAGAACCGGAUCUGCUACCUGCAGGAGAUCUUCUCCAACGAGAUGGCCAAGGUGGACGACUCCUUC |
| with ORF | | UUCCACCGGCUGGAGGAGUCCUUCCUGGUGGAGGAGGACAAGAAGCACGAGCGGCACCCCAUCUUCGGCAACAUCGUGGACGAGGUGGCCUACCACG |
| with Hibit | | AGAAGUACCCCACCAUCUACCACCUGCGGAAGAAGCUGGUGGACUCCACCGACAAGGCCGACCUGCGGCUGAUCUACCUGGCCCUGGCCCACAUGAU |
| tag | | CAAGUUCCGGGGCCACUUCCUGAUCGAGGGCGACCUGAACCCCGACAACUCCGACGUGGACAAGCUGUUCAUCCAGCUGGUGCAGACCUACAACCAG |
| | | CUGUUCGAGGAGAACCCCAUCAACGCCUCCGGCGUGGACGCCAAGGCCAUCCUGUCCGCCCGGCUGUCCAAGUCCCGGCGGCUGGAGAACCUGAUCG |
| | | CCCAGCUGCCCGGCGAGAAGAAGAACGGCCUGUUCGGCAACCUGAUCGCCCUGUCCCUGGGCCUGACCCCCAACUUCAAGUCCAACUUCGACCUGGC |
| | | CGAGGACGCCAAGCUGCAGCUGUCCAAGGACACCUACGACGACGACCUGGACAACCUGCUGGCCCAGAUCGGCGACCAGUACGCCGACCUGUUCCUG |
| | | GCCGCCAAGAACCUGUCCGACGCCAUCCUGCUGUCCGACAUCCUGCGGGUGAACACCGAGAUCACCAAGGCCCCCCUGUCCGCCUCCAUGAUCAAGC |
| | | GGUACGACGAGCACCACCAGGACCUGACCCUGCUGAAGGCCCUGGUGCGGCAGCAGCUGCCCGAGAAGUACAAGGAGAUCUUCUUCGACCAGUCCAA |
| | | GAACGGCUACGCCGGCUACAUCGACGGCGGCGCCUCCCAGGAGGAGUUCUACAAGUUCAUCAAGCCCAUCCUGGAGAAGAUGGACGGCACCGAGGAG |
| | | CUGCUGGUGAAGCUGAACCGGGAGGACCUGCUGCGGAAGCAGCGGACCUUCGACAACGGCUCCAUCCCCCACCAGAUCCACCUGGGCGAGCUGCACG |
| | | CCAUCCUGCGGCGGCAGGAGGACUUCUACCCCUUCCUGAAGGACAACCGGGAGAAGAUCGAGAAGAUCCUGACCUUCCGGAUCCCCUACUACGUGGG |
| | | CCCCCUGGCCCGGGGCAACUCCCGGUUCGCCUGGAUGACCCGGAAGUCCGAGGAGACCAUCACCCCCUGGAACUUCGAGGAGGUGGUGGACAAGGGC |
| | | GCCUCCGCCCAGUCCUUCAUCGAGCGGAUGACCAACUUCGACAAGAACCUGCCCAACGAGAAGGUGCUGCCCAAGCACUCCCUGCUGUACGAGUACU |
| | | UCACCGUGUACAACGAGCUGACCAAGGUGAAGUACGUGACCGAGGGCAUGCGGAAGCCCGCCUUCCUGUCCGGCGAGCAGAAGAAGGCCAUCGUGGA |
| | | CCUGCUGUUCAAGACCAACCGGAAGGUGACCGUGAAGCAGCUGAAGGAGGACUACUUCAAGAAGAUCGAGUGCUUCGACUCCGUGGAGAUCUCCGGC |
| | | GUGGAGGACCGGUUCAACGCCUCCCUGGGCACCUACCACGACCUGCUGAAGAUCAUCAAGGACAAGGACUUCCUGGACAACGAGGAGAACGAGGACA |
| | | UCCUGGAGGACAUCGUGCUGACCCUGACCCUGUUCGAGGACCGGGAGAUGAUCGAGGAGCGGCUGAAGACCUACGCCCACCUGUUCGACGACAAGGU |
| | | GAUGAAGCAGCUGAAGCGGCGGCGGUACACCGGCUGGGGCCGGCUGUCCCGGAAGCUGAUCAACGGCAUCCGGGACAAGCAGUCCGGCAAGACCAUC |
| | | CUGGACUUCCUGAAGUCCGACGGCUUCGCCAACCGGAACUUCAUGCAGCUGAUCCACGACGACUCCCUGACCUUCAAGGAGGACAUCCAGAAGGCCC |
| | | AGGUGUCCGGCCAGGGCGACUCCCUGCACGAGCACAUCGCCAACCUGGCCGGCUCCCCCGCCAUCAAGAAGGGCAUCCUGCAGACCGUGAAGGUGGU |
| | | GGACGAGCUGGUGAAGGUGAUGGGCCGGCACAAGCCCGAGAACAUCGUGAUCGAGAUGGCCCGGGAGAACCAGACCACCCAGAAGGGCCAGAAGAAC |
| | | UCCCGGGAGCGGAUGAAGCGGAUCGAGGAGGGCAUCAAGGAGCUGGGCUCCCAGAUCCUGAAGGAGCACCCCGUGGAGAACACCCAGCUGCAGAACG |
| | | AGAAGCUGUACCUGUACUACCUGCAGAACGGCCGGGACAUGUACGUGGACCAGGAGCUGGACAUCAACCGGCUGUCCGACUACGACGUGGACCACAU |
| | | CGUGCCCCAGUCCUUCCUGAAGGACGACUCCAUCGACAACAAGGUGCUGACCCGGUCCGACAAGAACCGGGGCAAGUCCGACAACGUGCCCUCCGAG |
| | | GAGGUGGUGAAGAAGAUGAAGAACUACUGGCGGCAGCUGCUGAACGCCAAGCUGAUCACCCAGCGGAAGUUCGACAACCUGACCAAGGCCGAGCGGG |
| | | GCGGCCUGUCCGAGCUGGACAAGGCCGGCUUCAUCAAGCGGCAGCUGGUGGAGACCCGGCAGAUCACCAAGCACGUGGCCCAGAUCCUGGACUCCCG |
| | | GAUGAACACCAAGUACGACGAGAACGACAAGCUGAUCCGGGAGGUGAAGGUGAUCACCCUGAAGUCCAAGCUGGUGUCCGACUUCCGGAAGGACUUC |
| | | CAGUUCUACAAGGUGCGGGAGAUCAACAACUACCACCACGCCCACGACGCCUACCUGAACGCCGUGGUGGGCACCGCCCUGAUCAAGAAGUACCCCA |
| | | AGCUGGAGUCCGAGUUCGUGUACGGCGACUACAAGGUGUACGACGUGCGGAAGAUGAUCGCCAAGUCCGAGCAGGAGAUCGGCAAGGCCACCGCCAA |
| | | GUACUUCUUCUACUCCAACAUCAUGAACUUCUUCAAGACCGAGAUCACCCUGGCCAACGGCGAGAUCCGGAAGCGGCCCCUGAUCGAGACCAACGGC |
| | | GAGACCGGCGAGAUCGUGUGGGACAAGGGCCGGGACUUCGCCACCGUGCGGAAGGUGCUGUCCAUGCCCCAGGUGAACAUCGUGAAGAAGACCGAGG |
| | | UGCAGACCGGCGGCUUCUCCAAGGAGUCCAUCCUGCCCAAGCGGAACUCCGACAAGCUGAUCGCCCGGAAGAAGGACUGGGACCCCAAGAAGUACGG |
| | | CGGCUUCGACUCCCCCACCGUGGCCUACUCCGUGCUGGUGGUGGCCAAGGUGGAGAAGGGCAAGUCCAAGAAGCUGAAGUCCGUGAAGGAGCUGCUG |
| | | GGCAUCACCAUCAUGGAGCGGUCCUCCUUCGAGAAGAACCCCAUCGACUUCCUGGAGGCCAAGGGCUACAAGGAGGUGAAGAAGGACCUGAUCAUCA |
| | | AGCUGCCCAAGUACUCCCUGUUCGAGCUGGAGAACGGCCGGAAGCGGAUGCUGGCCUCCGCCGGCGAGCUGCAGAAGGGCAACGAGCUGGCCCUGCC |
| | | CUCCAAGUACGUGAACUUCCUGUACCUGGCCUCCCACUACGAGAAGCUGAAGGGCUCCCCCGAGGACAACGAGCAGAAGCAGCUGUUCGUGGAGCAG |
| | | CACAAGCACUACCUGGACGAGAUCAUCGAGCAGAUCUCCGAGUUCUCCAAGCGGGUGAUCCUGGCCGACGCCAACCUGGACAAGGUGCUGUCCGCCU |
| | | ACAACAAGCACCGGGACAAGCCCAUCCGGGAGCAGGCCGAGAACAUCAUCCACCUGUUCACCCUGACCAACCUGGGCGCCCCCGCCGCCUUCAAGUA |
| | | CUUCGACACCACCAUCGACCGGAAGCGGUACACCUCCACCAAGGAGGUGCUGGACGCCACCCUGAUCCACCAGUCCAUCACCGGCCUGUACGAGACC |
| | | CGGAUCGACCUGUCCCAGCUGGGCGGCGACGGCGGCGGCUCCCCCAAGAAGAAGCGGAAGGUGUCCGAGUCCGCCACCCCCGAGUCCGUGUCCGGCU |
| | | GGCGGCUGUUCAAGAAGAUCUCCUGA |
| Amino acid | 350 | MTGAAFKPNPINYILGLDIGIASVGWAMVEIDEEENPIRLIDLGVRVFERAEVPKTGDSLAMARRLARSVRRLTRRRAHRLLRARRLLKREGVLQAA |
| sequence | | DFDENGLIKSLPNTPWQLRAAALDRKLTPLEWSAVLLHLIKHRGYLSQRKNEGETADKELGALLKGVANNAHALQTGDFRTPAELALNKFEKESGHI |
| for | | RNQRGDYSHTFSRKDLQAELILLFEKQKEFGNPHVSGGLKEGIETLLMTQRPALSGDAVQKMLGHCTFEPAEPKAAKNTYTAERFIWLTKLNNLRIL |
| Nme2Cas9 | | EQGSERPLTDTERATLMDEPYRKSKLTYAQARKLLGLEDTAFFKGLRYGKDNAEASTLMEMKAYHAISRALEKEGLKDKKSPLNLSSELQDEIGTAF |
| encoded by | | SLFKTDEDITGRLKDRVQPEILEALLKHISFDKFVQISLKALRRIVPLMEQGKRYDEACAEIYGDHYGKKNTEEKIYLPPIPADEIRNPVVLRALSQ |
| mRNA C | | ARKVINGVVRRYGSPARIHIETAREVGKSFKDRKEIEKRQEENRKDREKAAAKFREYFPNFVGEPKSKDILKLRLYEQQHGKCLYSGKEINLVRLNE |
| | | KGYVEIDHALPFSRTWDDSFNNKVLVLGSENQNKGNQTPYEYFNGKDNSREWQEFKARVETSRFPRSKKQRILLQKFDEDGFKECNLNDTRYVNRFL |
| | | CQFVADHILLTGKGKRRVFASNGQITNLLRGFWGLRKVRAENDRHHALDAVVVACSTVAMQQKITRFVRYKEMNAFDGKTIDKETGKVLHQKTHFPQ |
| | | PWEFFAQEVMIRVFGKPDGKPEFEEADTPEKLRTLLAEKLSSRPEAVHEYVTPLFVSRAPNRKMSGAHKDTLRSAKRFVKHNEKISVKRVWLTEIKL |
| | | ADLENMVNYKNGREIELYEALKARLEAYGGNAKQAFDPKDNPFYKKGGQLVKAVRVEKTQESGVLLNKKNAYTIADNGDMVRVDVFCKVDKKGKNQY |
| | | FIVPIYAWQVAENILPDIDCKGYRIDDSYTFCFSLHKYDLIAFQKDEKSKVEFAYYINCDSSNGRFYLAWHDKGSKEQQFRISTQNLVLIQKYQVNE |
| | | LGKEIRPCRLKKRPPVRSGKRTADGSEFESPKKKRKVE* |
| Amino acid | 351 | MTNLSDIIEKETGKQLVIQESILMLPEEVEEVIGNKPESDILVHTAYDESTDENVMLLTSDAPEYKPWALVIQDSNGENKIKMLSGGSKRTADGSEF |
| sequence | | ESPKKKRKVE** |
| for UGI | | |
| encoded by | | |
| mRNA G | | |
| Amino acid | 352 | MVPKKKRKVAAFKPNPINYILGLDIGIASVGWAMVEIDEEENPIRLIDLGVRVFERAEVPKTGDSLAMARRLARSVRRLTRRRAHRLLRARRLLKRE |
| sequence | | GVLQAADFDENGLIKSLPNTPWQLRAAALDRKLTPLEWSAVLLHLIKHRGYLSQRKNEGETADKELGALLKGVANNAHALQTGDFRTPAELALNKFE |
| for | | KESGHIRNQRGDYSHTFSRKDLQAELILLFEKQKEFGNPHVSGGLKEGIETLLMTQRPALSGDAVQKMLGHCTFEPAEPKAAKNTYTAERFIWLTKL |
| Nme2Cas9 | | NNLRILEQGSERPLTDTERATLMDEPYRKSKLTYAQARKLLGLEDTAFFKGLRYGKDNAEASTLMEMKAYHAISRALEKEGLKDKKSPLNLSSELQD |
| encoded by | | EIGTAFSLFKTDEDITGRLKDRVQPEILEALLKHISFDKFVQISLKALRRIVPLMEQGKRYDEACAEIYGDHYGKKNTEEKIYLPPIPADEIRNPVV |
| mRNA I | | LRALSQARKVINGVVRRYGSPARIHIETAREVGKSFKDRKEIEKRQEENRKDREKAAAKFREYFPNFVGEPKSKDILKLRLYEQQHGKCLYSGKEIN |
| | | LVRLNEKGYVEIDHALPFSRTWDDSENNKVLVLGSENQNKGNQTPYEYENGKDNSREWQEFKARVETSRFPRSKKQRILLQKFDEDGFKECNLNDTR |
| | | YVNRFLCQFVADHILLTGKGKRRVFASNGQITNLLRGFWGLRKVRAENDRHHALDAVVVACSTVAMQQKITRFVRYKEMNAFDGKTIDKETGKVLHQ |
| | | KTHFPQPWEFFAQEVMIRVFGKPDGKPEFEEADTPEKLRTLLAEKLSSRPEAVHEYVTPLFVSRAPNRKMSGAHKDTLRSAKRFVKHNEKISVKRVW |
| | | LTEIKLADLENMVNYKNGREIELYEALKARLEAYGGNAKQAFDPKDNPFYKKGGQLVKAVRVEKTQESGVLLNKKNAYTIADNGDMVRVDVFCKVDK |
| | | KGKNQYFIVPIYAWQVAENILPDIDCKGYRIDDSYTFCFSLHKYDLIAFQKDEKSKVEFAYYINCDSSNGRFYLAWHDKGSKEQQFRISTQNLVLIQ |
| | | KYQVNELGKEIRPCRLKKRPPVRYPYDVPDYAAAPAAKKKKLD* |
| Amino acid | 353 | MAAFKPNPINYILGLDIGIASVGWAMVEIDEEENPIRLIDLGVRVFERAEVPKTGDSLAMARRLARSVRRLTRRRAHRLLRARRLLKREGVLQAADF |
| sequence | | DENGLIKSLPNTPWQLRAAALDRKLTPLEWSAVLLHLIKHRGYLSQRKNEGETADKELGALLKGVANNAHALQTGDERTPAELALNKFEKESGHIRN |
| for | | QRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQ |
| Nme2Cas9 | | GSERPLTDTERATLMDEPYRKSKLTYAQARKLLGLEDTAFFKGLRYGKDNAEASTLMEMKAYHAISRALEKEGLKDKKSPLNLSSELQDEIGTAFSL |
| encoded by | | FKTDEDITGRLKDRVQPEILEALLKHISFDKFVQISLKALRRIVPLMEQGKRYDEACAEIYGDHYGKKNTEEKIYLPPIPADEIRNPVVLRALSQAR |
| mRNA J | | KVINGVVRRYGSPARIHIETAREVGKSFKDRKEIEKRQEENRKDREKAAAKFREYFPNFVGEPKSKDILKLRLYEQQHGKCLYSGKEINLVRLNEKG |
| | | YVEIDHALPFSRTWDDSFNNKVLVLGSENQNKGNQTPYEYENGKDNSREWQEFKARVETSRFPRSKKQRILLQKFDEDGFKECNLNDTRYVNRFLCQ |
| | | FVADHILLTGKGKRRVFASNGQITNLLRGFWGLRKVRAENDRHHALDAVVVACSTVAMQQKITRFVRYKEMNAFDGKTIDKETGKVLHQKTHFPQPW |
| | | EFFAQEVMIRVFGKPDGKPEFEEADTPEKLRTLLAEKLSSRPEAVHEYVTPLFVSRAPNRKMSGAHKDTLRSAKRFVKHNEKISVKRVWLTEIKLAD |
| | | LENMVNYKNGREIELYEALKARLEAYGGNAKQAFDPKDNPFYKKGGQLVKAVRVEKTQESGVLLNKKNAYTIADNGDMVRVDVFCKVDKKGKNQYFI |
| | | VPIYAWQVAENILPDIDCKGYRIDDSYTFCFSLHKYDLIAFQKDEKSKVEFAYYINCDSSNGRFYLAWHDKGSKEQQFRISTQNLVLIQKYQVNELG |
| | | KEIRPCRLKKRPPVR |
| Amino acid | 354 | MDGSGGGSPKKKRKVGGSGGGAAFKPNPINYILGLDIGIASVGWAMVEIDEEENPIRLIDLGVRVFERAEVPKTGDSLAMARRLARSVRRLTRRRAH |
| sequence | | RLLRARRLLKREGVLQAADFDENGLIKSLPNTPWQLRAAALDRKLTPLEWSAVLLHLIKHRGYLSQRKNEGETADKELGALLKGVANNAHALQTGDF |
| for | | RTPAELALNKFEKESGHIRNQRGDYSHTFSRKDLQAELILLFEKQKEFGNPHVSGGLKEGIETLLMTQRPALSGDAVQKMLGHCTFEPAEPKAAKNT |
| Nme2Cas9 | | YTAERFIWLTKLNNLRILEQGSERPLTDTERATLMDEPYRKSKLTYAQARKLLGLEDTAFFKGLRYGKDNAEASTLMEMKAYHAISRALEKEGLKDK |
| encoded by | | KSPLNLSSELQDEIGTAFSLEKTDEDITGRLKDRVQPEILEALLKHISFDKFVQISLKALRRIVPLMEQGKRYDEACAEIYGDHYGKKNTEEKIYLP |
| mRNA M | | PIPADEIRNPVVLRALSQARKVINGVVRRYGSPARIHIETAREVGKSFKDRKEIEKRQEENRKDREKAAAKFREYFPNFVGEPKSKDILKLRLYEQQ |
| | | HGKCLYSGKEINLVRLNEKGYVEIDHALPFSRTWDDSENNKVLVLGSENQNKGNQTPYEYENGKDNSREWQEFKARVETSRFPRSKKQRILLQKFDE |
| | | DGFKECNLNDTRYVNRFLCQFVADHILLTGKGKRRVFASNGQITNLLRGFWGLRKVRAENDRHHALDAVVVACSTVAMQQKITRFVRYKEMNAFDGK |
| | | TIDKETGKVLHQKTHFPQPWEFFAQEVMIRVFGKPDGKPEFEEADTPEKLRTLLAEKLSSRPEAVHEYVTPLFVSRAPNRKMSGAHKDTLRSAKRFV |
| | | KHNEKISVKRVWLTEIKLADLENMVNYKNGREIELYEALKARLEAYGGNAKQAFDPKDNPFYKKGGQLVKAVRVEKTQESGVLLNKKNAYTIADNGD |
| | | MVRVDVFCKVDKKGKNQYFIVPIYAWQVAENILPDIDCKGYRIDDSYTFCFSLHKYDLIAFQKDEKSKVEFAYYINCDSSNGRFYLAWHDKGSKEQQ |
| | | FRISTQNLVLIQKYQVNELGKEIRPCRLKKRPPVRSESATPESVSGWRLFKKIS* |
| Amino acid | 355 | MDGSGGGSPKKKRKVGGSGGGAAFKPNPINYILGLDIGIASVGWAMVEIDEEENPIRLIDLGVRVFERAEVPKTGDSLAMARRLARSVRRLTRRRAH |
| sequence | | RLLRARRLLKREGVLQAADEDENGLIKSLPNTPWQLRAAALDRKLTPLEWSAVLLHLIKHRGYLSQRKNEGETADKELGALLKGVANNAHALQTGDF |
| for | | RTPAELALNKFEKESGHIRNQRGDYSHTFSRKDLQAELILLFEKQKEFGNPHVSGGLKEGIETLLMTQRPALSGDAVQKMLGHCTFEPAEPKAAKNT |
| Nme2Cas9 | | YTAERFIWLTKLNNLRILEQGSERPLTDTERATLMDEPYRKSKLTYAQARKLLGLEDTAFFKGLRYGKDNAEASTLMEMKAYHAISRALEKEGLKDK |
| encoded by | | KSPLNLSSELQDEIGTAFSLFKTDEDITGRLKDRVQPEILEALLKHISFDKFVQISLKALRRIVPLMEQGKRYDEACAEIYGDHYGKKNTEEKIYLP |
| mRNA N | | PIPADEIRNPVVLRALSQARKVINGVVRRYGSPARIHIETAREVGKSFKDRKEIEKRQEENRKDREKAAAKFREYFPNFVGEPKSKDILKLRLYEQQ |
| | | HGKCLYSGKEINLVRLNEKGYVEIDHALPFSRTWDDSENNKVLVLGSENQNKGNQTPYEYENGKDNSREWQEFKARVETSRFPRSKKQRILLQKFDE |
| | | DGFKECNLNDTRYVNRFLCQFVADHILLTGKGKRRVFASNGQITNLLRGFWGLRKVRAENDRHHALDAVVVACSTVAMQQKITRFVRYKEMNAFDGK |
| | | TIDKETGKVLHQKTHFPQPWEFFAQEVMIRVFGKPDGKPEFEEADTPEKLRTLLAEKLSSRPEAVHEYVTPLFVSRAPNRKMSGAHKDTLRSAKRFV |
| | | KHNEKISVKRVWLTEIKLADLENMVNYKNGREIELYEALKARLEAYGGNAKQAFDPKDNPFYKKGGQLVKAVRVEKTQESGVLLNKKNAYTIADNGD |
| | | MVRVDVFCKVDKKGKNQYFIVPIYAWQVAENILPDIDCKGYRIDDSYTFCFSLHKYDLIAFQKDEKSKVEFAYYINCDSSNGRFYLAWHDKGSKEQQ |
| | | FRISTQNLVLIQKYQVNELGKEIRPCRLKKRPPVRSGKRTADGSGGGSPAAKKKKLD* |
| Amino acid | 356 | MDGSGGGSPKKKRKVEDKRPAATKKAGQAKKKKGGSGGGAAFKPNPINYILGLDIGIASVGWAMVEIDEEENPIRLIDLGVRVFERAEVPKTGDSLA |
| sequence | | MARRLARSVRRLTRRRAHRLLRARRLLKREGVLQAADFDENGLIKSLPNTPWQLRAAALDRKLTPLEWSAVLLHLIKHRGYLSQRKNEGETADKELG |
| for | | ALLKGVANNAHALQTGDFRTPAELALNKFEKESGHIRNQRGDYSHTFSRKDLQAELILLFEKQKEFGNPHVSGGLKEGIETLLMTQRPALSGDAVQK |
| Nme2Cas9 | | MLGHCTFEPAEPKAAKNTYTAERFIWLTKLNNLRILEQGSERPLTDTERATLMDEPYRKSKLTYAQARKLLGLEDTAFFKGLRYGKDNAEASTLMEM |
| encoded by | | KAYHAISRALEKEGLKDKKSPLNLSSELQDEIGTAFSLFKTDEDITGRLKDRVQPEILEALLKHISFDKFVQISLKALRRIVPLMEQGKRYDEACAE |
| mRNA O | | IYGDHYGKKNTEEKIYLPPIPADEIRNPVVLRALSQARKVINGVVRRYGSPARIHIETAREVGKSFKDRKEIEKRQEENRKDREKAAAKFREYFPNF |
| | | VGEPKSKDILKLRLYEQQHGKCLYSGKEINLVRLNEKGYVEIDHALPFSRTWDDSFNNKVLVLGSENQNKGNQTPYEYENGKDNSREWQEFKARVET |
| | | SRFPRSKKQRILLQKFDEDGEKECNLNDTRYVNRFLCQFVADHILLTGKGKRRVFASNGQITNLLRGFWGLRKVRAENDRHHALDAVVVACSTVAMQ |
| | | QKITRFVRYKEMNAFDGKTIDKETGKVLHQKTHFPQPWEFFAQEVMIRVFGKPDGKPEFEEADTPEKLRTLLAEKLSSRPEAVHEYVTPLFVSRAPN |
| | | RKMSGAHKDTLRSAKRFVKHNEKISVKRVWLTEIKLADLENMVNYKNGREIELYEALKARLEAYGGNAKQAFDPKDNPFYKKGGQLVKAVRVEKTQE |
| | | SGVLLNKKNAYTIADNGDMVRVDVFCKVDKKGKNQYFIVPIYAWQVAENILPDIDCKGYRIDDSYTFCFSLHKYDLIAFQKDEKSKVEFAYYINCDS |
| | | SNGRFYLAWHDKGSKEQQFRISTQNLVLIQKYQVNELGKEIRPCRLKKRPPVR |
| Amino acid | 357 | MDGSGGGSPKKKRKVEDKRPAATKKAGQAKKKKGGSGGGAAFKPNPINYILGLDIGIASVGWAMVEIDEEENPIRLIDLGVRVFERAEVPKTGDSLA |
| sequence | | MARRLARSVRRLTRRRAHRLLRARRLLKREGVLQAADEDENGLIKSLPNTPWQLRAAALDRKLTPLEWSAVLLHLIKHRGYLSQRKNEGETADKELG |
| for | | ALLKGVANNAHALQTGDFRTPAELALNKFEKESGHIRNQRGDYSHTFSRKDLQAELILLFEKQKEFGNPHVSGGLKEGIETLLMTQRPALSGDAVQK |
| Nme2Cas9 | | MLGHCTFEPAEPKAAKNTYTAERFIWLTKLNNLRILEQGSERPLTDTERATLMDEPYRKSKLTYAQARKLLGLEDTAFFKGLRYGKDNAEASTLMEM |
| encoded by | | KAYHAISRALEKEGLKDKKSPLNLSSELQDEIGTAFSLEKTDEDITGRLKDRVQPEILEALLKHISFDKFVQISLKALRRIVPLMEQGKRYDEACAE |
| mRNA P | | IYGDHYGKKNTEEKIYLPPIPADEIRNPVVLRALSQARKVINGVVRRYGSPARIHIETAREVGKSFKDRKEIEKRQEENRKDREKAAAKFREYFPNF |
| | | VGEPKSKDILKLRLYEQQHGKCLYSGKEINLVRLNEKGYVEIDHALPFSRTWDDSFNNKVLVLGSENQNKGNQTPYEYENGKDNSREWQEFKARVET |
| | | SRFPRSKKQRILLQKFDEDGFKECNLNDTRYVNRFLCQFVADHILLTGKGKRRVFASNGQITNLLRGFWGLRKVRAENDRHHALDAVVVACSTVAMQ |
| | | QKITRFVRYKEMNAFDGKTIDKETGKVLHQKTHEPQPWEFFAQEVMIRVFGKPDGKPEFEEADTPEKLRTLLAEKLSSRPEAVHEYVTPLFVSRAPN |
| | | RKMSGAHKDTLRSAKRFVKHNEKISVKRVWLTEIKLADLENMVNYKNGREIELYEALKARLEAYGGNAKQAFDPKDNPFYKKGGQLVKAVRVEKTQE |
| | | SGVLLNKKNAYTIADNGDMVRVDVFCKVDKKGKNQYFIVPIYAWQVAENILPDIDCKGYRIDDSYTFCFSLHKYDLIAFQKDEKSKVEFAYYINCDS |
| | | SNGRFYLAWHDKGSKEQQFRISTQNLVLIQKYQVNELGKEIRPCRLKKRPPVRSESATPESVSGWRLFKKIS |
| Amino acid | 358 | MDGSGGGSEDKRPAATKKAGQAKKKKGGSGGGAAFKPNPINYILGLDIGIASVGWAMVEIDEEENPIRLIDLGVRVFERAEVPKTGDSLAMARRLAR |
| sequence | | SVRRLTRRRAHRLLRARRLLKREGVLQAADFDENGLIKSLPNTPWQLRAAALDRKLTPLEWSAVLLHLIKHRGYLSQRKNEGETADKELGALLKGVA |
| for | | NNAHALQTGDFRTPAELALNKFEKESGHIRNQRGDYSHTFSRKDLQAELILLFEKQKEFGNPHVSGGLKEGIETLLMTQRPALSGDAVQKMLGHCTF |
| Nme2Cas9 | | EPAEPKAAKNTYTAERFIWLTKLNNLRILEQGSERPLTDTERATLMDEPYRKSKLTYAQARKLLGLEDTAFFKGLRYGKDNAEASTLMEMKAYHAIS |
| encoded by | | RALEKEGLKDKKSPLNLSSELQDEIGTAFSLEKTDEDITGRLKDRVQPEILEALLKHISFDKFVQISLKALRRIVPLMEQGKRYDEACAEIYGDHYG |
| mRNA Q | | KKNTEEKIYLPPIPADEIRNPVVLRALSQARKVINGVVRRYGSPARIHIETAREVGKSFKDRKEIEKRQEENRKDREKAAAKFREYFPNFVGEPKSK |
| | | DILKLRLYEQQHGKCLYSGKEINLVRLNEKGYVEIDHALPFSRTWDDSENNKVLVLGSENQNKGNQTPYEYENGKDNSREWQEFKARVETSRFPRSK |
| | | KQRILLQKFDEDGFKECNLNDTRYVNRFLCQFVADHILLTGKGKRRVFASNGQITNLLRGFWGLRKVRAENDRHHALDAVVVACSTVAMQQKITRFV |
| | | RYKEMNAFDGKTIDKETGKVLHQKTHFPQPWEFFAQEVMIRVFGKPDGKPEFEEADTPEKLRTLLAEKLSSRPEAVHEYVTPLFVSRAPNRKMSGAH |
| | | KDTLRSAKRFVKHNEKISVKRVWLTEIKLADLENMVNYKNGREIELYEALKARLEAYGGNAKQAFDPKDNPFYKKGGQLVKAVRVEKTQESGVLINK |
| | | KNAYTIADNGDMVRVDVFCKVDKKGKNQYFIVPIYAWQVAENILPDIDCKGYRIDDSYTFCFSLHKYDLIAFQKDEKSKVEFAYYINCDSSNGRFYL |
| | | AWHDKGSKEQQFRISTQNLVLIQKYQVNELGKEIRPCRLKKRPPVR |
| Amino acid | 359 | MDGSGGGSPKKKRKVEDKRPAATKKAGQAKKKKGGSGGGEASPASGPRHLMDPHIFTSNENNGIGRHKTYLCYEVERLDNGTSVKMDQHRGFLHNQA |
| sequence | | KNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYD |
| for | | EFKHCWDTFVDHQGCPFQPWDGLDEHSQALSGRLRAILQNQGNSGSETPGTSESATPESAAFKPNPINYILGLAIGIASVGWAMVEIDEEENPIRLI |
| Nme2Cas9 | | DLGVRVFERAEVPKTGDSLAMARRLARSVRRLTRRRAHRLLRARRLLKREGVLQAADFDENGLIKSLPNTPWQLRAAALDRKLTPLEWSAVLLHLIK |
| base editor | | HRGYLSQRKNEGETADKELGALLKGVANNAHALQTGDFRTPAELALNKFEKESGHIRNQRGDYSHTFSRKDLQAELILLFEKQKEFGNPHVSGGLKE |
| encoded by | | GIETLLMTQRPALSGDAVQKMLGHCTFEPAEPKAAKNTYTAERFIWLTKLNNLRILEQGSERPLTDTERATLMDEPYRKSKLTYAQARKLLGLEDTA |
| mRNA S | | FFKGLRYGKDNAEASTLMEMKAYHAISRALEKEGLKDKKSPLNLSSELQDEIGTAFSLFKTDEDITGRLKDRVQPEILEALLKHISFDKFVQISLKA |
| | | LRRIVPLMEQGKRYDEACAEIYGDHYGKKNTEEKIYLPPIPADEIRNPVVLRALSQARKVINGVVRRYGSPARIHIETAREVGKSFKDRKEIEKRQE |
| | | ENRKDREKAAAKFREYFPNFVGEPKSKDILKLRLYEQQHGKCLYSGKEINLVRLNEKGYVEIDHALPFSRTWDDSENNKVLVLGSENQNKGNQTPYE |
| | | YFNGKDNSREWQEFKARVETSRFPRSKKQRILLQKFDEDGFKECNLNDTRYVNRFLCQFVADHILLTGKGKRRVFASNGQITNLLRGFWGLRKVRAE |
| | | NDRHHALDAVVVACSTVAMQQKITRFVRYKEMNAFDGKTIDKETGKVLHQKTHFPQPWEFFAQEVMIRVFGKPDGKPEFEEADTPEKLRTLLAEKLS |
| | | SRPEAVHEYVTPLFVSRAPNRKMSGAHKDTLRSAKRFVKHNEKISVKRVWLTEIKLADLENMVNYKNGREIELYEALKARLEAYGGNAKQAFDPKDN |
| | | PFYKKGGQLVKAVRVEKTQESGVLLNKKNAYTIADNGDMVRVDVFCKVDKKGKNQYFIVPIYAWQVAENILPDIDCKGYRIDDSYTFCFSLHKYDLI |
| | | AFQKDEKSKVEFAYYINCDSSNGRFYLAWHDKGSKEQQFRISTQNLVLIQKYQVNELGKEIRPCRLKKRPPVR* |
| Amino acid | 360 | MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFIS |
| sequence | | WSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAIL |
| for BC22n | | QNQGNSGSETPGTSESATPESDKKYSIGLAIGTNSVGWAVITDEYKVPSKKFKVLGNTDRHSIKKNLIGALLFDSGETAEATRLKRTARRRYTRRKN |
| SpyCas9 | | RICYLQEIFSNEMAKVDDSFFHRLEESFLVEEDKKHERHPIFGNIVDEVAYHEKYPTIYHLRKKLVDSTDKADLRLIYLALAHMIKERGHFLIEGDL |
| base editor | | NPDNSDVDKLFIQLVQTYNQLFEENPINASGVDAKAILSARLSKSRRLENLIAQLPGEKKNGLFGNLIALSLGLTPNEKSNEDLAEDAKLQLSKDTY |
| encoded by | | DDDLDNLLAQIGDQYADLFLAAKNLSDAILLSDILRVNTEITKAPLSASMIKRYDEHHQDLTLLKALVRQQLPEKYKEIFFDQSKNGYAGYIDGGAS |
| mRNA E | | QEEFYKFIKPILEKMDGTEELLVKLNREDLLRKQRTEDNGSIPHQIHLGELHAILRRQEDFYPFLKDNREKIEKILTFRIPYYVGPLARGNSRFAWM |
| | | TRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVK |
| | | QLKEDYFKKIECFDSVEISGVEDRENASLGTYHDLLKIIKDKDELDNEENEDILEDIVLTLTLFEDREMIEERLKTYAHLEDDKVMKQLKRRRYTGW |
| | | GRLSRKLINGIRDKQSGKTILDFLKSDGFANRNFMQLIHDDSLTFKEDIQKAQVSGQGDSLHEHIANLAGSPAIKKGILQTVKVVDELVKVMGRHKP |
| | | ENIVIEMARENQTTQKGQKNSRERMKRIEEGIKELGSQILKEHPVENTQLQNEKLYLYYLQNGRDMYVDQELDINRLSDYDVDHIVPQSFLKDDSID |
| | | NKVLTRSDKNRGKSDNVPSEEVVKKMKNYWRQLLNAKLITQRKFDNLTKAERGGLSELDKAGFIKRQLVETRQITKHVAQILDSRMNTKYDENDKLI |
| | | REVKVITLKSKLVSDERKDFQFYKVREINNYHHAHDAYLNAVVGTALIKKYPKLESEFVYGDYKVYDVRKMIAKSEQEIGKATAKYFFYSNIMNFFK |
| | | TEITLANGEIRKRPLIETNGETGEIVWDKGRDFATVRKVLSMPQVNIVKKTEVQTGGFSKESILPKRNSDKLIARKKDWDPKKYGGEDSPTVAYSVL |
| | | VVAKVEKGKSKKLKSVKELLGITIMERSSFEKNPIDFLEAKGYKEVKKDLIIKLPKYSLFELENGRKRMLASAGELQKGNELALPSKYVNFLYLASH |
| | | YEKLKGSPEDNEQKQLFVEQHKHYLDEIIEQISEFSKRVILADANLDKVLSAYNKHRDKPIREQAENIIHLFTLTNLGAPAAFKYFDTTIDRKRYTS |
| | | TKEVLDATLIHQSITGLYETRIDLSQLGGDGGGSPKKKRKV* |
| mRNA C | 361 | GGGAAGCUCAGAAUAAACGCUCAACUUUGGCCGGAUCUGCCACCAUGACCGGUGCCGCCUUCAAGCCCAACCCCAUCAACUACAUCCUGGGCCUGGA |
| encoding | | CAUCGGCAUCGCCUCCGUGGGCUGGGCCAUGGUGGAGAUCGACGAGGAGGAGAACCCCAUCCGGCUGAUCGACCUGGGCGUGCGGGUGUUCGAGCGG |
| Nme2Cas 9 | | GCCGAGGUGCCCAAGACCGGCGACUCCCUGGCCAUGGCCCGGCGGCUGGCCCGGUCCGUGCGGCGGCUGACCCGGCGGCGGGCCCACCGGCUGCUGC |
| | | GGGCCCGGCGGCUGCUGAAGCGGGAGGGCGUGCUGCAGGCCGCCGACUUCGACGAGAACGGCCUGAUCAAGUCCCUGCCCAACACCCCCUGGCAGCU |
| | | GCGGGCCGCCGCCCUGGACCGGAAGCUGACCCCCCUGGAGUGGUCCGCCGUGCUGCUGCACCUGAUCAAGCACCGGGGCUACCUGUCCCAGCGGAAG |
| | | AACGAGGGCGAGACCGCCGACAAGGAGCUGGGCGCCCUGCUGAAGGGCGUGGCCAACAACGCCCACGCCCUGCAGACCGGCGACUUCCGGACCCCCG |
| | | CCGAGCUGGCCCUGAACAAGUUCGAGAAGGAGUCCGGCCACAUCCGGAACCAGCGGGGCGACUACUCCCACACCUUCUCCCGGAAGGACCUGCAGGC |
| | | CGAGCUGAUCCUGCUGUUCGAGAAGCAGAAGGAGUUCGGCAACCCCCACGUGUCCGGCGGCCUGAAGGAGGGCAUCGAGACCCUGCUGAUGACCCAG |
| | | CGGCCCGCCCUGUCCGGCGACGCCGUGCAGAAGAUGCUGGGCCACUGCACCUUCGAGCCCGCCGAGCCCAAGGCCGCCAAGAACACCUACACCGCCG |
| | | AGCGGUUCAUCUGGCUGACCAAGCUGAACAACCUGCGGAUCCUGGAGCAGGGCUCCGAGCGGCCCCUGACCGACACCGAGCGGGCCACCCUGAUGGA |
| | | CGAGCCCUACCGGAAGUCCAAGCUGACCUACGCCCAGGCCCGGAAGCUGCUGGGCCUGGAGGACACCGCCUUCUUCAAGGGCCUGCGGUACGGCAAG |
| | | GACAACGCCGAGGCCUCCACCCUGAUGGAGAUGAAGGCCUACCACGCCAUCUCCCGGGCCCUGGAGAAGGAGGGCCUGAAGGACAAGAAGUCCCCCC |
| | | UGAACCUGUCCUCCGAGCUGCAGGACGAGAUCGGCACCGCCUUCUCCCUGUUCAAGACCGACGAGGACAUCACCGGCCGGCUGAAGGACCGGGUGCA |
| | | GCCCGAGAUCCUGGAGGCCCUGCUGAAGCACAUCUCCUUCGACAAGUUCGUGCAGAUCUCCCUGAAGGCCCUGCGGCGGAUCGUGCCCCUGAUGGAG |
| | | CAGGGCAAGCGGUACGACGAGGCCUGCGCCGAGAUCUACGGCGACCACUACGGCAAGAAGAACACCGAGGAGAAGAUCUACCUGCCCCCCAUCCCCG |
| | | CCGACGAGAUCCGGAACCCCGUGGUGCUGCGGGCCCUGUCCCAGGCCCGGAAGGUGAUCAACGGCGUGGUGCGGCGGUACGGCUCCCCCGCCCGGAU |
| | | CCACAUCGAGACCGCCCGGGAGGUGGGCAAGUCCUUCAAGGACCGGAAGGAGAUCGAGAAGCGGCAGGAGGAGAACCGGAAGGACCGGGAGAAGGCC |
| | | GCCGCCAAGUUCCGGGAGUACUUCCCCAACUUCGUGGGCGAGCCCAAGUCCAAGGACAUCCUGAAGCUGCGGCUGUACGAGCAGCAGCACGGCAAGU |
| | | GCCUGUACUCCGGCAAGGAGAUCAACCUGGUGCGGCUGAACGAGAAGGGCUACGUGGAGAUCGACCACGCCCUGCCCUUCUCCCGGACCUGGGACGA |
| | | CUCCUUCAACAACAAGGUGCUGGUGCUGGGCUCCGAGAACCAGAACAAGGGCAACCAGACCCCCUACGAGUACUUCAACGGCAAGGACAACUCCCGG |
| | | GAGUGGCAGGAGUUCAAGGCCCGGGUGGAGACCUCCCGGUUCCCCCGGUCCAAGAAGCAGCGGAUCCUGCUGCAGAAGUUCGACGAGGACGGCUUCA |
| | | AGGAGUGCAACCUGAACGACACCCGGUACGUGAACCGCUUCCUGUGCCAGUUCGUGGCCGACCACAUCCUGCUGACCGGCAAGGGCAAGCGGCGGGU |
| | | GUUCGCCUCCAACGGCCAGAUCACCAACCUGCUGCGGGGCUUCUGGGGCCUGCGGAAGGUGCGGGCCGAGAACGACCGGCACCACGCCCUGGACGCC |
| | | GUGGUGGUGGCCUGCUCCACCGUGGCCAUGCAGCAGAAGAUCACCCGGUUCGUGCGGUACAAGGAGAUGAACGCCUUCGACGGCAAGACCAUCGACA |
| | | AGGAGACCGGCAAGGUGCUGCACCAGAAGACCCACUUCCCCCAGCCCUGGGAGUUCUUCGCCCAGGAGGUGAUGAUCCGGGUGUUCGGCAAGCCCGA |
| | | CGGCAAGCCCGAGUUCGAGGAGGCCGACACCCCCGAGAAGCUGCGGACCCUGCUGGCCGAGAAGCUGUCCUCCCGGCCCGAGGCCGUGCACGAGUAC |
| | | GUGACCCCCCUGUUCGUGUCCCGGGCCCCCAACCGGAAGAUGUCCGGCGCCCACAAGGACACCCUGCGGUCCGCCAAGCGGUUCGUGAAGCACAACG |
| | | AGAAGAUCUCCGUGAAGCGGGUGUGGCUGACCGAGAUCAAGCUGGCCGACCUGGAGAACAUGGUGAACUACAAGAACGGCCGGGAGAUCGAGCUGUA |
| | | CGAGGCCCUGAAGGCCCGGCUGGAGGCCUACGGCGGCAACGCCAAGCAGGCCUUCGACCCCAAGGACAACCCCUUCUACAAGAAGGGCGGCCAGCUG |
| | | GUGAAGGCCGUGCGGGUGGAGAAGACCCAGGAGUCCGGCGUGCUGCUGAACAAGAAGAACGCCUACACCAUCGCCGACAACGGCGACAUGGUGCGGG |
| | | UGGACGUGUUCUGCAAGGUGGACAAGAAGGGCAAGAACCAGUACUUCAUCGUGCCCAUCUACGCCUGGCAGGUGGCCGAGAACAUCCUGCCCGACAU |
| | | CGACUGCAAGGGCUACCGGAUCGACGACUCCUACACCUUCUGCUUCUCCCUGCACAAGUACGACCUGAUCGCCUUCCAGAAGGACGAGAAGUCCAAG |
| | | GUGGAGUUCGCCUACUACAUCAACUGCGACUCCUCCAACGGCCGGUUCUACCUGGCCUGGCACGACAAGGGCUCCAAGGAGCAGCAGUUCCGGAUCU |
| | | CCACCCAGAACCUGGUGCUGAUCCAGAAGUACCAGGUGAACGAGCUGGGCAAGGAGAUCCGGCCCUGCCGGCUGAAGAAGCGGCCCCCCGUGCGGUC |
| | | CGGAAAGCGGACCGCCGACGGCUCCGAGUUCGAGUCCCCCAAGAAGAAGCGGAAGGUGGAGUAGCUAGCACCAGCCUCAAGAACACCCGAAUGGAGU |
| | | CUCUAAGCUACAUAAUACCAACUUACACUUUACAAAAUGUUGUCCCCCAAAAUGUAGCCAUUCGUAUCUGCUCCUAAUAAAAAGAAAGUUUCUUCAC |
| | | AUUCUCUCGAGAAAAAAAAAAAAUGGAAAAAAAAAAAACGGAAAAAAAAAAAAGGUAAAAAAAAAAAAUAUAAAAAAAAAAAACAUAAAAAAAAAAA |
| | | ACGAAAAAAAAAAAACGUAAAAAAAAAAAACUCAAAAAAAAAAAAGAUAAAAAAAAAAAACCUAAAAAAAAAAAAUGUAAAAAAAAAAAAGGGAAAA |
| | | AAAAAAAACGCAAAAAAAAAAAACACAAAAAAAAAAAAUGCAAAAAAAAAAAAUCGAAAAAAAAAAAAUCUAAAAAAAAAAAACGAAAAAAAAAAAA |
| | | CCCAAAAAAAAAAAAGACAAAAAAAAAAAAUAGAAAAAAAAAAAAGUUAAAAAA |
| mRNA G | 362 | GGGAAGCUCAGAAUAAACGCUCAACUUUGGCCGGAUCUGCCACCAUGACCAACCUGUCCGACAUCAUCGAGAAGGAGACCGGCAAGCAGCUGGUGAU |
| encoding | | CCAGGAGUCCAUCCUGAUGCUGCCCGAGGAGGUGGAGGAGGUGAUCGGCAACAAGCCCGAGUCCGACAUCCUGGUGCACACCGCCUACGACGAGUCC |
| UGI | | ACCGACGAGAACGUGAUGCUGCUGACCUCCGACGCCCCCGAGUACAAGCCCUGGGCCCUGGUGAUCCAGGACUCCAACGGCGAGAACAAGAUCAAGA |
| | | UGCUGUCCGGCGGCUCCAAGCGGACCGCCGACGGCUCCGAGUUCGAGUCCCCCAAGAAGAAGCGGAAGGUGGAGUGAUAGCUAGCACCAGCCUCAAG |
| | | AACACCCGAAUGGAGUCUCUAAGCUACAUAAUACCAACUUACACUUUACAAAAUGUUGUCCCCCAAAAUGUAGCCAUUCGUAUCUGCUCCUAAUAAA |
| | | AAGAAAGUUUCUUCACAUUCUCUCGAGAAAAAAAAAAAAUGGAAAAAAAAAAAACGGAAAAAAAAAAAAGGUAAAAAAAAAAAAUAUAAAAAAAAAA |
| | | AACAUAAAAAAAAAAAACGAAAAAAAAAAAACGUAAAAAAAAAAAACUCAAAAAAAAAAAAGAUAAAAAAAAAAAACCUAAAAAAAAAAAAUGUAAA |
| | | AAAAAAAAAGGGAAAAAAAAAAAACGCAAAAAAAAAAAACACAAAAAAAAAAAAUGCAAAAAAAAAAAAUCGAAAAAAAAAAAAUCUAAAAAAAAAA |
| | | AACGAAAAAAAAAAAACCCAAAAAAAAAAAAGACAAAAAAAAAAAAUAGAAAAAAAAAAAAGUUAAAAAAAAAAAACUGAAAAAAAAAAAAUUUAAA |
| | | AAAAAAAAAUCUAG |
| mRNA I | 363 | GGGaagctcagaataaacgctcaactttggccggatctgccacCATGGTGCCCAAGAAGAAGCGGAAGGTGGCCGCCTTCAAGCCCAACCCCATCAA |
| encoding | | CTACATCCTGGGCCTGGACATCGGCATCGCCTCCGTGGGCTGGGCCATGGTGGAGATCGACGAGGAGGAGAACCCCATCCGGCTGATCGACCTGGGC |
| Nme2Cas9 | | GTGCGGGTGTTCGAGCGGGCCGAGGTGCCCAAGACCGGCGACTCCCTGGCCATGGCCCGGCGGCTGGCCCGGTCCGTGCGGCGGCTGACCCGGCGGC |
| | | GGGCCCACCGGCTGCTGCGGGCCCGGCGGCTGCTGAAGCGGGAGGGCGTGCTGCAGGCCGCCGACTTCGACGAGAACGGCCTGATCAAGTCCCTGCC |
| | | CAACACCCCCTGGCAGCTGCGGGCCGCCGCCCTGGACCGGAAGCTGACCCCCCTGGAGTGGTCCGCCGTGCTGCTGCACCTGATCAAGCACCGGGGC |
| | | TACCTGTCCCAGCGGAAGAACGAGGGCGAGACCGCCGACAAGGAGCTGGGCGCCCTGCTGAAGGGCGTGGCCAACAACGCCCACGCCCTGCAGACCG |
| | | GCGACTTCCGGACCCCCGCCGAGCTGGCCCTGAACAAGTTCGAGAAGGAGTCCGGCCACATCCGGAACCAGCGGGGCGACTACTCCCACACCTTCTC |
| | | CCGGAAGGACCTGCAGGCCGAGCTGATCCTGCTGTTCGAGAAGCAGAAGGAGTTCGGCAACCCCCACGTGTCCGGCGGCCTGAAGGAGGGCATCGAG |
| | | ACCCTGCTGATGACCCAGCGGCCCGCCCTGTCCGGCGACGCCGTGCAGAAGATGCTGGGCCACTGCACCTTCGAGCCCGCCGAGCCCAAGGCCGCCA |
| | | AGAACACCTACACCGCCGAGCGGTTCATCTGGCTGACCAAGCTGAACAACCTGCGGATCCTGGAGCAGGGCTCCGAGCGGCCCCTGACCGACACCGA |
| | | GCGGGCCACCCTGATGGACGAGCCCTACCGGAAGTCCAAGCTGACCTACGCCCAGGCCCGGAAGCTGCTGGGCCTGGAGGACACCGCCTTCTTCAAG |
| | | GGCCTGCGGTACGGCAAGGACAACGCCGAGGCCTCCACCCTGATGGAGATGAAGGCCTACCACGCCATCTCCCGGGCCCTGGAGAAGGAGGGCCTGA |
| | | AGGACAAGAAGTCCCCCCTGAACCTGTCCTCCGAGCTGCAGGACGAGATCGGCACCGCCTTCTCCCTGTTCAAGACCGACGAGGACATCACCGGCCG |
| | | GCTGAAGGACCGGGTGCAGCCCGAGATCCTGGAGGCCCTGCTGAAGCACATCTCCTTCGACAAGTTCGTGCAGATCTCCCTGAAGGCCCTGCGGCGG |
| | | ATCGTGCCCCTGATGGAGCAGGGCAAGCGGTACGACGAGGCCTGCGCCGAGATCTACGGCGACCACTACGGCAAGAAGAACACCGAGGAGAAGATCT |
| | | ACCTGCCCCCCATCCCCGCCGACGAGATCCGGAACCCCGTGGTGCTGCGGGCCCTGTCCCAGGCCCGGAAGGTGATCAACGGCGTGGTGCGGCGGTA |
| | | CGGCTCCCCCGCCCGGATCCACATCGAGACCGCCCGGGAGGTGGGCAAGTCCTTCAAGGACCGGAAGGAGATCGAGAAGCGGCAGGAGGAGAACCGG |
| | | AAGGACCGGGAGAAGGCCGCCGCCAAGTTCCGGGAGTACTTCCCCAACTTCGTGGGCGAGCCCAAGTCCAAGGACATCCTGAAGCTGCGGCTGTACG |
| | | AGCAGCAGCACGGCAAGTGCCTGTACTCCGGCAAGGAGATCAACCTGGTGCGGCTGAACGAGAAGGGCTACGTGGAGATCGACCACGCCCTGCCCTT |
| | | CTCCCGGACCTGGGACGACTCCTTCAACAACAAGGTGCTGGTGCTGGGCTCCGAGAACCAGAACAAGGGCAACCAGACCCCCTACGAGTACTTCAAC |
| | | GGCAAGGACAACTCCCGGGAGTGGCAGGAGTTCAAGGCCCGGGTGGAGACCTCCCGGTTCCCCCGGTCCAAGAAGCAGCGGATCCTGCTGCAGAAGT |
| | | TCGACGAGGACGGCTTCAAGGAGTGCAACCTGAACGACACCCGGTACGTGAACCGGTTCCTGTGCCAGTTCGTGGCCGACCACATCCTGCTGACCGG |
| | | CAAGGGCAAGCGGCGGGTGTTCGCCTCCAACGGCCAGATCACCAACCTGCTGCGGGGCTTCTGGGGCCTGCGGAAGGTGCGGGCCGAGAACGACCGG |
| | | CACCACGCCCTGGACGCCGTGGTGGTGGCCTGCTCCACCGTGGCCATGCAGCAGAAGATCACCCGGTTCGTGCGGTACAAGGAGATGAACGCCTTCG |
| | | ACGGCAAGACCATCGACAAGGAGACCGGCAAGGTGCTGCACCAGAAGACCCACTTCCCCCAGCCCTGGGAGTTCTTCGCCCAGGAGGTGATGATCCG |
| | | GGTGTTCGGCAAGCCCGACGGCAAGCCCGAGTTCGAGGAGGCCGACACCCCCGAGAAGCTGCGGACCCTGCTGGCCGAGAAGCTGTCCTCCCGGCCC |
| | | GAGGCCGTGCACGAGTACGTGACCCCCCTGTTCGTGTCCCGGGCCCCCAACCGGAAGATGTCCGGCGCCCACAAGGACACCCTGCGGTCCGCCAAGC |
| | | GGTTCGTGAAGCACAACGAGAAGATCTCCGTGAAGCGGGTGTGGCTGACCGAGATCAAGCTGGCCGACCTGGAGAACATGGTGAACTACAAGAACGG |
| | | CCGGGAGATCGAGCTGTACGAGGCCCTGAAGGCCCGGCTGGAGGCCTACGGCGGCAACGCCAAGCAGGCCTTCGACCCCAAGGACAACCCCTTCTAC |
| | | AAGAAGGGCGGCCAGCTGGTGAAGGCCGTGCGGGTGGAGAAGACCCAGGAGTCCGGCGTGCTGCTGAACAAGAAGAACGCCTACACCATCGCCGACA |
| | | ACGGCGACATGGTGCGGGTGGACGTGTTCTGCAAGGTGGACAAGAAGGGCAAGAACCAGTACTTCATCGTGCCCATCTACGCCTGGCAGGTGGCCGA |
| | | GAACATCCTGCCCGACATCGACTGCAAGGGCTACCGGATCGACGACTCCTACACCTTCTGCTTCTCCCTGCACAAGTACGACCTGATCGCCTTCCAG |
| | | AAGGACGAGAAGTCCAAGGTGGAGTTCGCCTACTACATCAACTGCGACTCCTCCAACGGCCGGTTCTACCTGGCCTGGCACGACAAGGGCTCCAAGG |
| | | AGCAGCAGTTCCGGATCTCCACCCAGAACCTGGTGCTGATCCAGAAGTACCAGGTGAACGAGCTGGGCAAGGAGATCCGGCCCTGCCGGCTGAAGAA |
| | | GCGGCCCCCCGTGCGGTACCCCTACGACGTGCCCGACTACGCCGCCGCCCCCGCCGCCAAGAAGAAGAAGCTGGACTAGCTAGCaccagcctcaaga |
| | | acacccgaatggagtctctaagctacataataccaacttacactttacaaaatgttgtcccccaaaatgtagccattcgtatctgctcctaataaaa |
| | | agaaagtttcttcacattctCTCGAGAAAAAAAAAAAATGGAAAAAAAAAAAACGGAAAAAAAAAAAAGGTAAAAAAAAAAAATATAAAAAAAAAAA |
| | | ACATAAAAAAAAAAAACGAAAAAAAAAAAACGTAAAAAAAAAAAACTCAAAAAAAAAAAAGATAAAAAAAAAAAACCTAAAAAAAAAAAATGTAAAA |
| | | AAAAAAAAGGGAAAAAAAAAAAACGCAAAAAAAAAAAACACAAAAAAAAAAAATGCAAAAAAAAAAAATCGAAAAAAAAAAAATCTAAAAAAAAAAA |
| | | ACGAAAAAAAAAAAACCCAAAAAAAAAAAAGACAAAAAAAAAAAATAGAAAAAAAAAAAAGTTAAAAAAAAAAAACTGAAAAAAAAAAAATTTAAAA |
| | | AAAAAAAAT |
| mRNA J | 364 | GGGAAGCUCAGAAUAAACGCUCAACUUUGGCCGGAUCUGCCACCAUGGUGCCCAAGAAGAAGCGGAAGGUGGAGGACAAGCGGCCCGCCGCCACCAA |
| encoding | | GAAGGCCGGCCAGGCCAAGAAGAAGAAGAUGGCCGCCUUCAAGCCCAACCCCAUCAACUACAUCCUGGGCCUGGACAUCGGCAUCGCCUCCGUGGGC |
| Nme2Cas9 | | UGGGCCAUGGUGGAGAUCGACGAGGAGGAGAACCCCAUCCGGCUGAUCGACCUGGGCGUGCGGGUGUUCGAGCGGGCCGAGGUGCCCAAGACCGGCG |
| | | ACUCCCUGGCCAUGGCCCGGCGGCUGGCCCGGUCCGUGCGGCGGCUGACCCGGCGGCGGGCCCACCGGCUGCUGCGGGCCCGGCGGCUGCUGAAGCG |
| | | GGAGGGCGUGCUGCAGGCCGCCGACUUCGACGAGAACGGCCUGAUCAAGUCCCUGCCCAACACCCCCUGGCAGCUGCGGGCCGCCGCCCUGGACCGG |
| | | AAGCUGACCCCCCUGGAGUGGUCCGCCGUGCUGCUGCACCUGAUCAAGCACCGGGGCUACCUGUCCCAGCGGAAGAACGAGGGCGAGACCGCCGACA |
| | | AGGAGCUGGGCGCCCUGCUGAAGGGCGUGGCCAACAACGCCCACGCCCUGCAGACCGGCGACUUCCGGACCCCCGCCGAGCUGGCCCUGAACAAGUU |
| | | CGAGAAGGAGUCCGGCCACAUCCGGAACCAGCGGGGCGACUACUCCCACACCUUCUCCCGGAAGGACCUGCAGGCCGAGCUGAUCCUGCUGUUCGAG |
| | | AAGCAGAAGGAGUUCGGCAACCCCCACGUGUCCGGCGGCCUGAAGGAGGGCAUCGAGACCCUGCUGAUGACCCAGCGGCCCGCCCUGUCCGGCGACG |
| | | CCGUGCAGAAGAUGCUGGGCCACUGCACCUUCGAGCCCGCCGAGCCCAAGGCCGCCAAGAACACCUACACCGCCGAGCGGUUCAUCUGGCUGACCAA |
| | | GCUGAACAACCUGCGGAUCCUGGAGCAGGGCUCCGAGCGGCCCCUGACCGACACCGAGCGGGCCACCCUGAUGGACGAGCCCUACCGGAAGUCCAAG |
| | | CUGACCUACGCCCAGGCCCGGAAGCUGCUGGGCCUGGAGGACACCGCCUUCUUCAAGGGCCUGCGGUACGGCAAGGACAACGCCGAGGCCUCCACCC |
| | | UGAUGGAGAUGAAGGCCUACCACGCCAUCUCCCGGGCCCUGGAGAAGGAGGGCCUGAAGGACAAGAAGUCCCCCCUGAACCUGUCCUCCGAGCUGCA |
| | | GGACGAGAUCGGCACCGCCUUCUCCCUGUUCAAGACCGACGAGGACAUCACCGGCCGGCUGAAGGACCGGGUGCAGCCCGAGAUCCUGGAGGCCCUG |
| | | CUGAAGCACAUCUCCUUCGACAAGUUCGUGCAGAUCUCCCUGAAGGCCCUGCGGCGGAUCGUGCCCCUGAUGGAGCAGGGCAAGCGGUACGACGAGG |
| | | CCUGCGCCGAGAUCUACGGCGACCACUACGGCAAGAAGAACACCGAGGAGAAGAUCUACCUGCCCCCCAUCCCCGCCGACGAGAUCCGGAACCCCGU |
| | | GGUGCUGCGGGCCCUGUCCCAGGCCCGGAAGGUGAUCAACGGCGUGGUGCGGCGGUACGGCUCCCCCGCCCGGAUCCACAUCGAGACCGCCCGGGAG |
| | | GUGGGCAAGUCCUUCAAGGACCGGAAGGAGAUCGAGAAGCGGCAGGAGGAGAACCGGAAGGACCGGGAGAAGGCCGCCGCCAAGUUCCGGGAGUACU |
| | | UCCCCAACUUCGUGGGCGAGCCCAAGUCCAAGGACAUCCUGAAGCUGCGGCUGUACGAGCAGCAGCACGGCAAGUGCCUGUACUCCGGCAAGGAGAU |
| | | CAACCUGGUGCGGCUGAACGAGAAGGGCUACGUGGAGAUCGACCACGCCCUGCCCUUCUCCCGGACCUGGGACGACUCCUUCAACAACAAGGUGCUG |
| | | GUGCUGGGCUCCGAGAACCAGAACAAGGGCAACCAGACCCCCUACGAGUACUUCAACGGCAAGGACAACUCCCGGGAGUGGCAGGAGUUCAAGGCCC |
| | | GGGUGGAGACCUCCCGGUUCCCCCGGUCCAAGAAGCAGCGGAUCCUGCUGCAGAAGUUCGACGAGGACGGCUUCAAGGAGUGCAACCUGAACGACAC |
| | | CCGGUACGUGAACCGGUUCCUGUGCCAGUUCGUGGCCGACCACAUCCUGCUGACCGGCAAGGGCAAGCGGCGGGUGUUCGCCUCCAACGGCCAGAUC |
| | | ACCAACCUGCUGCGGGGCUUCUGGGGCCUGCGGAAGGUGCGGGCCGAGAACGACCGGCACCACGCCCUGGACGCCGUGGUGGUGGCCUGCUCCACCG |
| | | UGGCCAUGCAGCAGAAGAUCACCCGGUUCGUGCGGUACAAGGAGAUGAACGCCUUCGACGGCAAGACCAUCGACAAGGAGACCGGCAAGGUGCUGCA |
| | | CCAGAAGACCCACUUCCCCCAGCCCUGGGAGUUCUUCGCCCAGGAGGUGAUGAUCCGGGUGUUCGGCAAGCCCGACGGCAAGCCCGAGUUCGAGGAG |
| | | GCCGACACCCCCGAGAAGCUGCGGACCCUGCUGGCCGAGAAGCUGUCCUCCCGGCCCGAGGCCGUGCACGAGUACGUGACCCCCCUGUUCGUGUCCC |
| | | GGGCCCCCAACCGGAAGAUGUCCGGCGCCCACAAGGACACCCUGCGGUCCGCCAAGCGGUUCGUGAAGCACAACGAGAAGAUCUCCGUGAAGCGGGU |
| | | GUGGCUGACCGAGAUCAAGCUGGCCGACCUGGAGAACAUGGUGAACUACAAGAACGGCCGGGAGAUCGAGCUGUACGAGGCCCUGAAGGCCCGGCUG |
| | | GAGGCCUACGGCGGCAACGCCAAGCAGGCCUUCGACCCCAAGGACAACCCCUUCUACAAGAAGGGCGGCCAGCUGGUGAAGGCCGUGCGGGUGGAGA |
| | | AGACCCAGGAGUCCGGCGUGCUGCUGAACAAGAAGAACGCCUACACCAUCGCCGACAACGGCGACAUGGUGCGGGUGGACGUGUUCUGCAAGGUGGA |
| | | CAAGAAGGGCAAGAACCAGUACUUCAUCGUGCCCAUCUACGCCUGGCAGGUGGCCGAGAACAUCCUGCCCGACAUCGACUGCAAGGGCUACCGGAUC |
| | | GACGACUCCUACACCUUCUGCUUCUCCCUGCACAAGUACGACCUGAUCGCCUUCCAGAAGGACGAGAAGUCCAAGGUGGAGUUCGCCUACUACAUCA |
| | | ACUGCGACUCCUCCAACGGCCGGUUCUACCUGGCCUGGCACGACAAGGGCUCCAAGGAGCAGCAGUUCCGGAUCUCCACCCAGAACCUGGUGCUGAU |
| | | CCAGAAGUACCAGGUGAACGAGCUGGGCAAGGAGAUCCGGCCCUGCCGGCUGAAGAAGCGGCCCCCCGUGCGGGAGGACAAGCGGCCCGCCGCCACC |
| | | AAGAAGGCCGGCCAGGCCAAGAAGAAGAAGUACCCCUACGACGUGCCCGACUACGCCGGCUACCCCUACGACGUGCCCGACUACGCCGGCUCCUACC |
| | | CCUACGACGUGCCCGACUACGCCGCCGCCCCCGCCGCCAAGAAGAAGAAGCUGGACUAGCUAGCACCAGCCUCAAGAACACCCGAAUGGAGUCUCUA |
| | | AGCUACAUAAUACCAACUUACACUUUACAAAAUGUUGUCCCCCAAAAUGUAGCCAUUCGUAUCUGCUCCUAAUAAAAAGAAAGUUUCUUCACAUUCU |
| | | CUCGAGAAAAAAAAAAAAUGGAAAAAAAAAAAACGGAAAAAAAAAAAAGGUAAAAAAAAAAAAUAUAAAAAAAAAAAACAUAAAAAAAAAAAACGAA |
| | | AAAAAAAAAACGUAAAAAAAAAAAACUCAAAAAAAAAAAAGAUAAAAAAAAAAAACCUAAAAAAAAAAAAUGUAAAAAAAAAAAAGGGAAAAAAAAA |
| | | AAACGCAAAAAAAAAAAACACAAAAAAAAAAAAUGCAAAAAAAAAAAAUCGAAAAAAAAAAAAUCUAAAAAAAAAAAACGAAAAAAAAAAAACCCAA |
| | | AAAAAAAAAAGACAAAAAAAAAAAAUAGAAAAAAAAAAAAGUUAAAAAAAAAAAACUGAAAAAAAAAAAAUUUAAAAAAAAAAAAUCUAG |
| mRNA M | 365 | GGGAAGCUCAGAAUAAACGCUCAACUUUGGCCGGAUCUGCCACCAUGGACGGCUCCGGCGGCGGCUCCCCCAAGAAGAAGCGGAAGGUGGGCGGCUC |
| encoding | | CGGCGGCGGCGCCGCCUUCAAGCCCAACCCCAUCAACUACAUCCUGGGCCUGGACAUCGGCAUCGCCUCCGUGGGCUGGGCCAUGGUGGAGAUCGAC |
| Nme2Cas9 | | GAGGAGGAGAACCCCAUCCGGCUGAUCGACCUGGGCGUGCGGGUGUUCGAGCGGGCCGAGGUGCCCAAGACCGGCGACUCCCUGGCCAUGGCCCGGC |
| | | GGCUGGCCCGGUCCGUGCGGCGGCUGACCCGGCGGCGGGCCCACCGGCUGCUGCGGGCCCGGCGGCUGCUGAAGCGGGAGGGCGUGCUGCAGGCCGC |
| | | CGACUUCGACGAGAACGGCCUGAUCAAGUCCCUGCCCAACACCCCCUGGCAGCUGCGGGCCGCCGCCCUGGACCGGAAGCUGACCCCCCUGGAGUGG |
| | | UCCGCCGUGCUGCUGCACCUGAUCAAGCACCGGGGCUACCUGUCCCAGCGGAAGAACGAGGGCGAGACCGCCGACAAGGAGCUGGGCGCCCUGCUGA |
| | | AGGGCGUGGCCAACAACGCCCACGCCCUGCAGACCGGCGACUUCCGGACCCCCGCCGAGCUGGCCCUGAACAAGUUCGAGAAGGAGUCCGGCCACAU |
| | | CCGGAACCAGCGGGGCGACUACUCCCACACCUUCUCCCGGAAGGACCUGCAGGCCGAGCUGAUCCUGCUGUUCGAGAAGCAGAAGGAGUUCGGCAAC |
| | | CCCCACGUGUCCGGCGGCCUGAAGGAGGGCAUCGAGACCCUGCUGAUGACCCAGCGGCCCGCCCUGUCCGGCGACGCCGUGCAGAAGAUGCUGGGCC |
| | | ACUGCACCUUCGAGCCCGCCGAGCCCAAGGCCGCCAAGAACACCUACACCGCCGAGCGGUUCAUCUGGCUGACCAAGCUGAACAACCUGCGGAUCCU |
| | | GGAGCAGGGCUCCGAGCGGCCCCUGACCGACACCGAGCGGGCCACCCUGAUGGACGAGCCCUACCGGAAGUCCAAGCUGACCUACGCCCAGGCCCGG |
| | | AAGCUGCUGGGCCUGGAGGACACCGCCUUCUUCAAGGGCCUGCGGUACGGCAAGGACAACGCCGAGGCCUCCACCCUGAUGGAGAUGAAGGCCUACC |
| | | ACGCCAUCUCCCGGGCCCUGGAGAAGGAGGGCCUGAAGGACAAGAAGUCCCCCCUGAACCUGUCCUCCGAGCUGCAGGACGAGAUCGGCACCGCCUU |
| | | CUCCCUGUUCAAGACCGACGAGGACAUCACCGGCCGGCUGAAGGACCGGGUGCAGCCCGAGAUCCUGGAGGCCCUGCUGAAGCACAUCUCCUUCGAC |
| | | AAGUUCGUGCAGAUCUCCCUGAAGGCCCUGCGGCGGAUCGUGCCCCUGAUGGAGCAGGGCAAGCGGUACGACGAGGCCUGCGCCGAGAUCUACGGCG |
| | | ACCACUACGGCAAGAAGAACACCGAGGAGAAGAUCUACCUGCCCCCCAUCCCCGCCGACGAGAUCCGGAACCCCGUGGUGCUGCGGGCCCUGUCCCA |
| | | GGCCCGGAAGGUGAUCAACGGCGUGGUGCGGCGGUACGGCUCCCCCGCCCGGAUCCACAUCGAGACCGCCCGGGAGGUGGGCAAGUCCUUCAAGGAC |
| | | CGGAAGGAGAUCGAGAAGCGGCAGGAGGAGAACCGGAAGGACCGGGAGAAGGCCGCCGCCAAGUUCCGGGAGUACUUCCCCAACUUCGUGGGCGAGC |
| | | CCAAGUCCAAGGACAUCCUGAAGCUGCGGCUGUACGAGCAGCAGCACGGCAAGUGCCUGUACUCCGGCAAGGAGAUCAACCUGGUGCGGCUGAACGA |
| | | GAAGGGCUACGUGGAGAUCGACCACGCCCUGCCCUUCUCCCGGACCUGGGACGACUCCUUCAACAACAAGGUGCUGGUGCUGGGCUCCGAGAACCAG |
| | | AACAAGGGCAACCAGACCCCCUACGAGUACUUCAACGGCAAGGACAACUCCCGGGAGUGGCAGGAGUUCAAGGCCCGGGUGGAGACCUCCCGGUUCC |
| | | CCCGGUCCAAGAAGCAGCGGAUCCUGCUGCAGAAGUUCGACGAGGACGGCUUCAAGGAGUGCAACCUGAACGACACCCGGUACGUGAACCGGUUCCU |
| | | GUGCCAGUUCGUGGCCGACCACAUCCUGCUGACCGGCAAGGGCAAGCGGCGGGUGUUCGCCUCCAACGGCCAGAUCACCAACCUGCUGCGGGGCUUC |
| | | UGGGGCCUGCGGAAGGUGCGGGCCGAGAACGACCGGCACCACGCCCUGGACGCCGUGGUGGUGGCCUGCUCCACCGUGGCCAUGCAGCAGAAGAUCA |
| | | CCCGGUUCGUGCGGUACAAGGAGAUGAACGCCUUCGACGGCAAGACCAUCGACAAGGAGACCGGCAAGGUGCUGCACCAGAAGACCCACUUCCCCCA |
| | | GCCCUGGGAGUUCUUCGCCCAGGAGGUGAUGAUCCGGGUGUUCGGCAAGCCCGACGGCAAGCCCGAGUUCGAGGAGGCCGACACCCCCGAGAAGCUG |
| | | CGGACCCUGCUGGCCGAGAAGCUGUCCUCCCGGCCCGAGGCCGUGCACGAGUACGUGACCCCCCUGUUCGUGUCCCGGGCCCCCAACCGGAAGAUGU |
| | | CCGGCGCCCACAAGGACACCCUGCGGUCCGCCAAGCGGUUCGUGAAGCACAACGAGAAGAUCUCCGUGAAGCGGGUGUGGCUGACCGAGAUCAAGCU |
| | | GGCCGACCUGGAGAACAUGGUGAACUACAAGAACGGCCGGGAGAUCGAGCUGUACGAGGCCCUGAAGGCCCGGCUGGAGGCCUACGGCGGCAACGCC |
| | | AAGCAGGCCUUCGACCCCAAGGACAACCCCUUCUACAAGAAGGGCGGCCAGCUGGUGAAGGCCGUGCGGGUGGAGAAGACCCAGGAGUCCGGCGUGC |
| | | UGCUGAACAAGAAGAACGCCUACACCAUCGCCGACAACGGCGACAUGGUGCGGGUGGACGUGUUCUGCAAGGUGGACAAGAAGGGCAAGAACCAGUA |
| | | CUUCAUCGUGCCCAUCUACGCCUGGCAGGUGGCCGAGAACAUCCUGCCCGACAUCGACUGCAAGGGCUACCGGAUCGACGACUCCUACACCUUCUGC |
| | | UUCUCCCUGCACAAGUACGACCUGAUCGCCUUCCAGAAGGACGAGAAGUCCAAGGUGGAGUUCGCCUACUACAUCAACUGCGACUCCUCCAACGGCC |
| | | GGUUCUACCUGGCCUGGCACGACAAGGGCUCCAAGGAGCAGCAGUUCCGGAUCUCCACCCAGAACCUGGUGCUGAUCCAGAAGUACCAGGUGAACGA |
| | | GCUGGGCAAGGAGAUCCGGCCCUGCCGGCUGAAGAAGCGGCCCCCCGUGCGGUCCGAGUCCGCCACCCCCGAGUCCGUGUCCGGCUGGCGGCUGUUC |
| | | AAGAAGAUCUCCUAGCUAGCACCAGCCUCAAGAACACCCGAAUGGAGUCUCUAAGCUACAUAAUACCAACUUACACUUUACAAAAUGUUGUCCCCCA |
| | | AAAUGUAGCCAUUCGUAUCUGCUCCUAAUAAAAAGAAAGUUUCUUCACAUUCUCUCGAGAAAAAAAAAAAAUGGAAAAAAAAAAAACGGAAAAAAAA |
| | | AAAAGGUAAAAAAAAAAAAUAUAAAAAAAAAAAACAUAAAAAAAAAAAACGAAAAAAAAAAAACGUAAAAAAAAAAAACUCAAAAAAAAAAAAGAUA |
| | | AAAAAAAAAAACCUAAAAAAAAAAAAUGUAAAAAAAAAAAAGGGAAAAAAAAAAAACGCAAAAAAAAAAAACACAAAAAAAAAAAAUGCAAAAAAAA |
| | | AAAAUCGAAAAAAAAAAAAUCUAAAAAAAAAAAACGAAAAAAAAAAAACCCAAAAAAAAAAAAGACAAAAAAAAAAAAUAGAAAAAAAAAAAAGUUA |
| | | AAAAAAAAAAACUGAAAAAAAAAAAAUUUAAAAAAAAAAAAUCUAG |
| mRNA N | 366 | GGGAAGCUCAGAAUAAACGCUCAACUUUGGCCGGAUCUGCCACCAUGGACGGCUCCGGCGGCGGCUCCCCCAAGAAGAAGCGGAAGGUGGGCGGCUC |
| encoding | | CGGCGGCGGCGCCGCCUUCAAGCCCAACCCCAUCAACUACAUCCUGGGCCUGGACAUCGGCAUCGCCUCCGUGGGCUGGGCCAUGGUGGAGAUCGAC |
| Nme2Cas9 | | GAGGAGGAGAACCCCAUCCGGCUGAUCGACCUGGGCGUGCGGGUGUUCGAGCGGGCCGAGGUGCCCAAGACCGGCGACUCCCUGGCCAUGGCCCGGC |
| | | GGCUGGCCCGGUCCGUGCGGCGGCUGACCCGGCGGCGGGCCCACCGGCUGCUGCGGGCCCGGCGGCUGCUGAAGCGGGAGGGCGUGCUGCAGGCCGC |
| | | CGACUUCGACGAGAACGGCCUGAUCAAGUCCCUGCCCAACACCCCCUGGCAGCUGCGGGCCGCCGCCCUGGACCGGAAGCUGACCCCCCUGGAGUGG |
| | | UCCGCCGUGCUGCUGCACCUGAUCAAGCACCGGGGCUACCUGUCCCAGCGGAAGAACGAGGGCGAGACCGCCGACAAGGAGCUGGGCGCCCUGCUGA |
| | | AGGGCGUGGCCAACAACGCCCACGCCCUGCAGACCGGCGACUUCCGGACCCCCGCCGAGCUGGCCCUGAACAAGUUCGAGAAGGAGUCCGGCCACAU |
| | | CCGGAACCAGCGGGGCGACUACUCCCACACCUUCUCCCGGAAGGACCUGCAGGCCGAGCUGAUCCUGCUGUUCGAGAAGCAGAAGGAGUUCGGCAAC |
| | | CCCCACGUGUCCGGCGGCCUGAAGGAGGGCAUCGAGACCCUGCUGAUGACCCAGCGGCCCGCCCUGUCCGGCGACGCCGUGCAGAAGAUGCUGGGCC |
| | | ACUGCACCUUCGAGCCCGCCGAGCCCAAGGCCGCCAAGAACACCUACACCGCCGAGCGGUUCAUCUGGCUGACCAAGCUGAACAACCUGCGGAUCCU |
| | | GGAGCAGGGCUCCGAGCGGCCCCUGACCGACACCGAGCGGGCCACCCUGAUGGACGAGCCCUACCGGAAGUCCAAGCUGACCUACGCCCAGGCCCGG |
| | | AAGCUGCUGGGCCUGGAGGACACCGCCUUCUUCAAGGGCCUGCGGUACGGCAAGGACAACGCCGAGGCCUCCACCCUGAUGGAGAUGAAGGCCUACC |
| | | ACGCCAUCUCCCGGGCCCUGGAGAAGGAGGGCCUGAAGGACAAGAAGUCCCCCCUGAACCUGUCCUCCGAGCUGCAGGACGAGAUCGGCACCGCCUU |
| | | CUCCCUGUUCAAGACCGACGAGGACAUCACCGGCCGGCUGAAGGACCGGGUGCAGCCCGAGAUCCUGGAGGCCCUGCUGAAGCACAUCUCCUUCGAC |
| | | AAGUUCGUGCAGAUCUCCCUGAAGGCCCUGCGGCGGAUCGUGCCCCUGAUGGAGCAGGGCAAGCGGUACGACGAGGCCUGCGCCGAGAUCUACGGCG |
| | | ACCACUACGGCAAGAAGAACACCGAGGAGAAGAUCUACCUGCCCCCCAUCCCCGCCGACGAGAUCCGGAACCCCGUGGUGCUGCGGGCCCUGUCCCA |
| | | GGCCCGGAAGGUGAUCAACGGCGUGGUGCGGCGGUACGGCUCCCCCGCCCGGAUCCACAUCGAGACCGCCCGGGAGGUGGGCAAGUCCUUCAAGGAC |
| | | CGGAAGGAGAUCGAGAAGCGGCAGGAGGAGAACCGGAAGGACCGGGAGAAGGCCGCCGCCAAGUUCCGGGAGUACUUCCCCAACUUCGUGGGCGAGC |
| | | CCAAGUCCAAGGACAUCCUGAAGCUGCGGCUGUACGAGCAGCAGCACGGCAAGUGCCUGUACUCCGGCAAGGAGAUCAACCUGGUGCGGCUGAACGA |
| | | GAAGGGCUACGUGGAGAUCGACCACGCCCUGCCCUUCUCCCGGACCUGGGACGACUCCUUCAACAACAAGGUGCUGGUGCUGGGCUCCGAGAACCAG |
| | | AACAAGGGCAACCAGACCCCCUACGAGUACUUCAACGGCAAGGACAACUCCCGGGAGUGGCAGGAGUUCAAGGCCCGGGUGGAGACCUCCCGGUUCC |
| | | CCCGGUCCAAGAAGCAGCGGAUCCUGCUGCAGAAGUUCGACGAGGACGGCUUCAAGGAGUGCAACCUGAACGACACCCGGUACGUGAACCGGUUCCU |
| | | GUGCCAGUUCGUGGCCGACCACAUCCUGCUGACCGGCAAGGGCAAGCGGCGGGUGUUCGCCUCCAACGGCCAGAUCACCAACCUGCUGCGGGGCUUC |
| | | UGGGGCCUGCGGAAGGUGCGGGCCGAGAACGACCGGCACCACGCCCUGGACGCCGUGGUGGUGGCCUGCUCCACCGUGGCCAUGCAGCAGAAGAUCA |
| | | CCCGGUUCGUGCGGUACAAGGAGAUGAACGCCUUCGACGGCAAGACCAUCGACAAGGAGACCGGCAAGGUGCUGCACCAGAAGACCCACUUCCCCCA |
| | | GCCCUGGGAGUUCUUCGCCCAGGAGGUGAUGAUCCGGGUGUUCGGCAAGCCCGACGGCAAGCCCGAGUUCGAGGAGGCCGACACCCCCGAGAAGCUG |
| | | CGGACCCUGCUGGCCGAGAAGCUGUCCUCCCGGCCCGAGGCCGUGCACGAGUACGUGACCCCCCUGUUCGUGUCCCGGGCCCCCAACCGGAAGAUGU |
| | | CCGGCGCCCACAAGGACACCCUGCGGUCCGCCAAGCGGUUCGUGAAGCACAACGAGAAGAUCUCCGUGAAGCGGGUGUGGCUGACCGAGAUCAAGCU |
| | | GGCCGACCUGGAGAACAUGGUGAACUACAAGAACGGCCGGGAGAUCGAGCUGUACGAGGCCCUGAAGGCCCGGCUGGAGGCCUACGGCGGCAACGCC |
| | | AAGCAGGCCUUCGACCCCAAGGACAACCCCUUCUACAAGAAGGGCGGCCAGCUGGUGAAGGCCGUGCGGGUGGAGAAGACCCAGGAGUCCGGCGUGC |
| | | UGCUGAACAAGAAGAACGCCUACACCAUCGCCGACAACGGCGACAUGGUGCGGGUGGACGUGUUCUGCAAGGUGGACAAGAAGGGCAAGAACCAGUA |
| | | CUUCAUCGUGCCCAUCUACGCCUGGCAGGUGGCCGAGAACAUCCUGCCCGACAUCGACUGCAAGGGCUACCGGAUCGACGACUCCUACACCUUCUGC |
| | | UUCUCCCUGCACAAGUACGACCUGAUCGCCUUCCAGAAGGACGAGAAGUCCAAGGUGGAGUUCGCCUACUACAUCAACUGCGACUCCUCCAACGGCC |
| | | GGUUCUACCUGGCCUGGCACGACAAGGGCUCCAAGGAGCAGCAGUUCCGGAUCUCCACCCAGAACCUGGUGCUGAUCCAGAAGUACCAGGUGAACGA |
| | | GCUGGGCAAGGAGAUCCGGCCCUGCCGGCUGAAGAAGCGGCCCCCCGUGCGGUCCGGAAAGCGGACCGCCGACGGCUCCGGAGGAGGAAGCCCCGCC |
| | | GCCAAGAAGAAGAAGCUGGACUAGCUAGCACCAGCCUCAAGAACACCCGAAUGGAGUCUCUAAGCUACAUAAUACCAACUUACACUUUACAAAAUGU |
| | | UGUCCCCCAAAAUGUAGCCAUUCGUAUCUGCUCCUAAUAAAAAGAAAGUUUCUUCACAUUCUCUCGAGAAAAAAAAAAAAUGGAAAAAAAAAAAACG |
| | | GAAAAAAAAAAAAGGUAAAAAAAAAAAAUAUAAAAAAAAAAAACAUAAAAAAAAAAAACGAAAAAAAAAAAACGUAAAAAAAAAAAACUCAAAAAAA |
| | | AAAAGAUAAAAAAAAAAAACCUAAAAAAAAAAAAUGUAAAAAAAAAAAAGGGAAAAAAAAAAAACGCAAAAAAAAAAAACACAAAAAAAAAAAAUGC |
| | | AAAAAAAAAAAAUCGAAAAAAAAAAAAUCUAAAAAAAAAAAACGAAAAAAAAAAAACCCAAAAAAAAAAAAGACAAAAAAAAAAAAUAGAAAAAAAA |
| | | AAAGUUAAAAAAAAAAAACUGAAAAAAAAAAAAUUUAAAAAAAAAAAAUCUAG |
| mRNA O | 367 | GGGAAGCUCAGAAUAAACGCUCAACUUUGGCCGGAUCUGCCACCAUGGACGGCUCCGGCGGCGGCUCCCCCAAGAAGAAGCGGAAGGUGGAGGACAA |
| encoding | | GCGGCCCGCCGCCACCAAGAAGGCCGGCCAGGCCAAGAAGAAGAAGGGCGGCUCCGGCGGCGGCGCCGCCUUCAAGCCCAACCCCAUCAACUACAUC |
| Nme2Cas9 | | CUGGGCCUGGACAUCGGCAUCGCCUCCGUGGGCUGGGCCAUGGUGGAGAUCGACGAGGAGGAGAACCCCAUCCGGCUGAUCGACCUGGGCGUGCGGG |
| | | UGUUCGAGCGGGCCGAGGUGCCCAAGACCGGCGACUCCCUGGCCAUGGCCCGGCGGCUGGCCCGGUCCGUGCGGCGGCUGACCCGGCGGCGGGCCCA |
| | | CCGGCUGCUGCGGGCCCGGCGGCUGCUGAAGCGGGAGGGCGUGCUGCAGGCCGCCGACUUCGACGAGAACGGCCUGAUCAAGUCCCUGCCCAACACC |
| | | CCCUGGCAGCUGCGGGCCGCCGCCCUGGACCGGAAGCUGACCCCCCUGGAGUGGUCCGCCGUGCUGCUGCACCUGAUCAAGCACCGGGGCUACCUGU |
| | | CCCAGCGGAAGAACGAGGGCGAGACCGCCGACAAGGAGCUGGGCGCCCUGCUGAAGGGCGUGGCCAACAACGCCCACGCCCUGCAGACCGGCGACUU |
| | | CCGGACCCCCGCCGAGCUGGCCCUGAACAAGUUCGAGAAGGAGUCCGGCCACAUCCGGAACCAGCGGGGCGACUACUCCCACACCUUCUCCCGGAAG |
| | | GACCUGCAGGCCGAGCUGAUCCUGCUGUUCGAGAAGCAGAAGGAGUUCGGCAACCCCCACGUGUCCGGCGGCCUGAAGGAGGGCAUCGAGACCCUGC |
| | | UGAUGACCCAGCGGCCCGCCCUGUCCGGCGACGCCGUGCAGAAGAUGCUGGGCCACUGCACCUUCGAGCCCGCCGAGCCCAAGGCCGCCAAGAACAC |
| | | CUACACCGCCGAGCGGUUCAUCUGGCUGACCAAGCUGAACAACCUGCGGAUCCUGGAGCAGGGCUCCGAGCGGCCCCUGACCGACACCGAGCGGGCC |
| | | ACCCUGAUGGACGAGCCCUACCGGAAGUCCAAGCUGACCUACGCCCAGGCCCGGAAGCUGCUGGGCCUGGAGGACACCGCCUUCUUCAAGGGCCUGC |
| | | GGUACGGCAAGGACAACGCCGAGGCCUCCACCCUGAUGGAGAUGAAGGCCUACCACGCCAUCUCCCGGGCCCUGGAGAAGGAGGGCCUGAAGGACAA |
| | | GAAGUCCCCCCUGAACCUGUCCUCCGAGCUGCAGGACGAGAUCGGCACCGCCUUCUCCCUGUUCAAGACCGACGAGGACAUCACCGGCCGGCUGAAG |
| | | GACCGGGUGCAGCCCGAGAUCCUGGAGGCCCUGCUGAAGCACAUCUCCUUCGACAAGUUCGUGCAGAUCUCCCUGAAGGCCCUGCGGCGGAUCGUGC |
| | | CCCUGAUGGAGCAGGGCAAGCGGUACGACGAGGCCUGCGCCGAGAUCUACGGCGACCACUACGGCAAGAAGAACACCGAGGAGAAGAUCUACCUGCC |
| | | CCCCAUCCCCGCCGACGAGAUCCGGAACCCCGUGGUGCUGCGGGCCCUGUCCCAGGCCCGGAAGGUGAUCAACGGCGUGGUGCGGCGGUACGGCUCC |
| | | CCCGCCCGGAUCCACAUCGAGACCGCCCGGGAGGUGGGCAAGUCCUUCAAGGACCGGAAGGAGAUCGAGAAGCGGCAGGAGGAGAACCGGAAGGACC |
| | | GGGAGAAGGCCGCCGCCAAGUUCCGGGAGUACUUCCCCAACUUCGUGGGCGAGCCCAAGUCCAAGGACAUCCUGAAGCUGCGGCUGUACGAGCAGCA |
| | | GCACGGCAAGUGCCUGUACUCCGGCAAGGAGAUCAACCUGGUGCGGCUGAACGAGAAGGGCUACGUGGAGAUCGACCACGCCCUGCCCUUCUCCCGG |
| | | ACCUGGGACGACUCCUUCAACAACAAGGUGCUGGUGCUGGGCUCCGAGAACCAGAACAAGGGCAACCAGACCCCCUACGAGUACUUCAACGGCAAGG |
| | | ACAACUCCCGGGAGUGGCAGGAGUUCAAGGCCCGGGUGGAGACCUCCCGGUUCCCCCGGUCCAAGAAGCAGCGGAUCCUGCUGCAGAAGUUCGACGA |
| | | GGACGGCUUCAAGGAGUGCAACCUGAACGACACCCGGUACGUGAACCGGUUCCUGUGCCAGUUCGUGGCCGACCACAUCCUGCUGACCGGCAAGGGC |
| | | AAGCGGCGGGUGUUCGCCUCCAACGGCCAGAUCACCAACCUGCUGCGGGGCUUCUGGGGCCUGCGGAAGGUGCGGGCCGAGAACGACCGGCACCACG |
| | | CCCUGGACGCCGUGGUGGUGGCCUGCUCCACCGUGGCCAUGCAGCAGAAGAUCACCCGGUUCGUGCGGUACAAGGAGAUGAACGCCUUCGACGGCAA |
| | | GACCAUCGACAAGGAGACCGGCAAGGUGCUGCACCAGAAGACCCACUUCCCCCAGCCCUGGGAGUUCUUCGCCCAGGAGGUGAUGAUCCGGGUGUUC |
| | | GGCAAGCCCGACGGCAAGCCCGAGUUCGAGGAGGCCGACACCCCCGAGAAGCUGCGGACCCUGCUGGCCGAGAAGCUGUCCUCCCGGCCCGAGGCCG |
| | | UGCACGAGUACGUGACCCCCCUGUUCGUGUCCCGGGCCCCCAACCGGAAGAUGUCCGGCGCCCACAAGGACACCCUGCGGUCCGCCAAGCGGUUCGU |
| | | GAAGCACAACGAGAAGAUCUCCGUGAAGCGGGUGUGGCUGACCGAGAUCAAGCUGGCCGACCUGGAGAACAUGGUGAACUACAAGAACGGCCGGGAG |
| | | AUCGAGCUGUACGAGGCCCUGAAGGCCCGGCUGGAGGCCUACGGCGGCAACGCCAAGCAGGCCUUCGACCCCAAGGACAACCCCUUCUACAAGAAGG |
| | | GCGGCCAGCUGGUGAAGGCCGUGCGGGUGGAGAAGACCCAGGAGUCCGGCGUGCUGCUGAACAAGAAGAACGCCUACACCAUCGCCGACAACGGCGA |
| | | CAUGGUGCGGGUGGACGUGUUCUGCAAGGUGGACAAGAAGGGCAAGAACCAGUACUUCAUCGUGCCCAUCUACGCCUGGCAGGUGGCCGAGAACAUC |
| | | CUGCCCGACAUCGACUGCAAGGGCUACCGGAUCGACGACUCCUACACCUUCUGCUUCUCCCUGCACAAGUACGACCUGAUCGCCUUCCAGAAGGACG |
| | | AGAAGUCCAAGGUGGAGUUCGCCUACUACAUCAACUGCGACUCCUCCAACGGCCGGUUCUACCUGGCCUGGCACGACAAGGGCUCCAAGGAGCAGCA |
| | | GUUCCGGAUCUCCACCCAGAACCUGGUGCUGAUCCAGAAGUACCAGGUGAACGAGCUGGGCAAGGAGAUCCGGCCCUGCCGGCUGAAGAAGCGGCCC |
| | | CCCGUGCGGUAGCUAGCACCAGCCUCAAGAACACCCGAAUGGAGUCUCUAAGCUACAUAAUACCAACUUACACUUUACAAAAUGUUGUCCCCCAAAA |
| | | UGUAGCCAUUCGUAUCUGCUCCUAAUAAAAAGAAAGUUUCUUCACAUUCUCUCGAGAAAAAAAAAAAAUGGAAAAAAAAAAAACGGAAAAAAAAAAA |
| | | AGGUAAAAAAAAAAAAUAUAAAAAAAAAAAACAUAAAAAAAAAAAACGAAAAAAAAAAAACGUAAAAAAAAAAAACUCAAAAAAAAAAAGAUAAAAA |
| | | AAAAAAACCUAAAAAAAAAAAAUGUAAAAAAAAAAAAGGGAAAAAAAAAAAACGCAAAAAAAAAAAACACAAAAAAAAAAAAUGCAAAAAAAAAAAA |
| | | UCGAAAAAAAAAAAAUCUAAAAAAAAAAAACGAAAAAAAAAAAACCCAAAAAAAAAAAAGACAAAAAAAAAAAAUAGAAAAAAAAAAAGUUAAAAAA |
| | | AAAAAACUGAAAAAAAAAAAAUUUAAAAAAAAAAAAUCUAG |
| mRNA P | 368 | GGGAAGCUCAGAAUAAACGCUCAACUUUGGCCGGAUCUGCCACCAUGGACGGCUCCGGCGGCGGCUCCCCCAAGAAGAAGCGGAAGGUGGAGGACAA |
| encoding | | GCGGCCCGCCGCCACCAAGAAGGCCGGCCAGGCCAAGAAGAAGAAGGGCGGCUCCGGCGGCGGCGCCGCCUUCAAGCCCAACCCCAUCAACUACAUC |
| Nme2Cas9 | | CUGGGCCUGGACAUCGGCAUCGCCUCCGUGGGCUGGGCCAUGGUGGAGAUCGACGAGGAGGAGAACCCCAUCCGGCUGAUCGACCUGGGCGUGCGGG |
| | | UGUUCGAGCGGGCCGAGGUGCCCAAGACCGGCGACUCCCUGGCCAUGGCCCGGCGGCUGGCCCGGUCCGUGCGGCGGCUGACCCGGCGGCGGGCCCA |
| | | CCGGCUGCUGCGGGCCCGGCGGCUGCUGAAGCGGGAGGGCGUGCUGCAGGCCGCCGACUUCGACGAGAACGGCCUGAUCAAGUCCCUGCCCAACACC |
| | | CCCUGGCAGCUGCGGGCCGCCGCCCUGGACCGGAAGCUGACCCCCCUGGAGUGGUCCGCCGUGCUGCUGCACCUGAUCAAGCACCGGGGCUACCUGU |
| | | CCCAGCGGAAGAACGAGGGCGAGACCGCCGACAAGGAGCUGGGCGCCCUGCUGAAGGGCGUGGCCAACAACGCCCACGCCCUGCAGACCGGCGACUU |
| | | CCGGACCCCCGCCGAGCUGGCCCUGAACAAGUUCGAGAAGGAGUCCGGCCACAUCCGGAACCAGCGGGGCGACUACUCCCACACCUUCUCCCGGAAG |
| | | GACCUGCAGGCCGAGCUGAUCCUGCUGUUCGAGAAGCAGAAGGAGUUCGGCAACCCCCACGUGUCCGGCGGCCUGAAGGAGGGCAUCGAGACCCUGC |
| | | UGAUGACCCAGCGGCCCGCCCUGUCCGGCGACGCCGUGCAGAAGAUGCUGGGCCACUGCACCUUCGAGCCCGCCGAGCCCAAGGCCGCCAAGAACAC |
| | | CUACACCGCCGAGCGGUUCAUCUGGCUGACCAAGCUGAACAACCUGCGGAUCCUGGAGCAGGGCUCCGAGCGGCCCCUGACCGACACCGAGCGGGCC |
| | | ACCCUGAUGGACGAGCCCUACCGGAAGUCCAAGCUGACCUACGCCCAGGCCCGGAAGCUGCUGGGCCUGGAGGACACCGCCUUCUUCAAGGGCCUGC |
| | | GGUACGGCAAGGACAACGCCGAGGCCUCCACCCUGAUGGAGAUGAAGGCCUACCACGCCAUCUCCCGGGCCCUGGAGAAGGAGGGCCUGAAGGACAA |
| | | GAAGUCCCCCCUGAACCUGUCCUCCGAGCUGCAGGACGAGAUCGGCACCGCCUUCUCCCUGUUCAAGACCGACGAGGACAUCACCGGCCGGCUGAAG |
| | | GACCGGGUGCAGCCCGAGAUCCUGGAGGCCCUGCUGAAGCACAUCUCCUUCGACAAGUUCGUGCAGAUCUCCCUGAAGGCCCUGCGGCGGAUCGUGC |
| | | CCCUGAUGGAGCAGGGCAAGCGGUACGACGAGGCCUGCGCCGAGAUCUACGGCGACCACUACGGCAAGAAGAACACCGAGGAGAAGAUCUACCUGCC |
| | | CCCCAUCCCCGCCGACGAGAUCCGGAACCCCGUGGUGCUGCGGGCCCUGUCCCAGGCCCGGAAGGUGAUCAACGGCGUGGUGCGGCGGUACGGCUCC |
| | | CCCGCCCGGAUCCACAUCGAGACCGCCCGGGAGGUGGGCAAGUCCUUCAAGGACCGGAAGGAGAUCGAGAAGCGGCAGGAGGAGAACCGGAAGGACC |
| | | GGGAGAAGGCCGCCGCCAAGUUCCGGGAGUACUUCCCCAACUUCGUGGGCGAGCCCAAGUCCAAGGACAUCCUGAAGCUGCGGCUGUACGAGCAGCA |
| | | GCACGGCAAGUGCCUGUACUCCGGCAAGGAGAUCAACCUGGUGCGGCUGAACGAGAAGGGCUACGUGGAGAUCGACCACGCCCUGCCCUUCUCCCGG |
| | | ACCUGGGACGACUCCUUCAACAACAAGGUGCUGGUGCUGGGCUCCGAGAACCAGAACAAGGGCAACCAGACCCCCUACGAGUACUUCAACGGCAAGG |
| | | ACAACUCCCGGGAGUGGCAGGAGUUCAAGGCCCGGGUGGAGACCUCCCGGUUCCCCCGGUCCAAGAAGCAGCGGAUCCUGCUGCAGAAGUUCGACGA |
| | | GGACGGCUUCAAGGAGUGCAACCUGAACGACACCCGGUACGUGAACCGGUUCCUGUGCCAGUUCGUGGCCGACCACAUCCUGCUGACCGGCAAGGGC |
| | | AAGCGGCGGGUGUUCGCCUCCAACGGCCAGAUCACCAACCUGCUGCGGGGCUUCUGGGGCCUGCGGAAGGUGCGGGCCGAGAACGACCGGCACCACG |
| | | CCCUGGACGCCGUGGUGGUGGCCUGCUCCACCGUGGCCAUGCAGCAGAAGAUCACCCGGUUCGUGCGGUACAAGGAGAUGAACGCCUUCGACGGCAA |
| | | GACCAUCGACAAGGAGACCGGCAAGGUGCUGCACCAGAAGACCCACUUCCCCCAGCCCUGGGAGUUCUUCGCCCAGGAGGUGAUGAUCCGGGUGUUC |
| | | GGCAAGCCCGACGGCAAGCCCGAGUUCGAGGAGGCCGACACCCCCGAGAAGCUGCGGACCCUGCUGGCCGAGAAGCUGUCCUCCCGGCCCGAGGCCG |
| | | UGCACGAGUACGUGACCCCCCUGUUCGUGUCCCGGGCCCCCAACCGGAAGAUGUCCGGCGCCCACAAGGACACCCUGCGGUCCGCCAAGCGGUUCGU |
| | | GAAGCACAACGAGAAGAUCUCCGUGAAGCGGGUGUGGCUGACCGAGAUCAAGCUGGCCGACCUGGAGAACAUGGUGAACUACAAGAACGGCCGGGAG |
| | | AUCGAGCUGUACGAGGCCCUGAAGGCCCGGCUGGAGGCCUACGGCGGCAACGCCAAGCAGGCCUUCGACCCCAAGGACAACCCCUUCUACAAGAAGG |
| | | GCGGCCAGCUGGUGAAGGCCGUGCGGGUGGAGAAGACCCAGGAGUCCGGCGUGCUGCUGAACAAGAAGAACGCCUACACCAUCGCCGACAACGGCGA |
| | | CAUGGUGCGGGUGGACGUGUUCUGCAAGGUGGACAAGAAGGGCAAGAACCAGUACUUCAUCGUGCCCAUCUACGCCUGGCAGGUGGCCGAGAACAUC |
| | | CUGCCCGACAUCGACUGCAAGGGCUACCGGAUCGACGACUCCUACACCUUCUGCUUCUCCCUGCACAAGUACGACCUGAUCGCCUUCCAGAAGGACG |
| | | AGAAGUCCAAGGUGGAGUUCGCCUACUACAUCAACUGCGACUCCUCCAACGGCCGGUUCUACCUGGCCUGGCACGACAAGGGCUCCAAGGAGCAGCA |
| | | GUUCCGGAUCUCCACCCAGAACCUGGUGCUGAUCCAGAAGUACCAGGUGAACGAGCUGGGCAAGGAGAUCCGGCCCUGCCGGCUGAAGAAGCGGCCC |
| | | CCCGUGCGGUCCGAGUCCGCCACCCCCGAGUCCGUGUCCGGCUGGCGGCUGUUCAAGAAGAUCUCCUAGCUAGCACCAGCCUCAAGAACACCCGAAU |
| | | GGAGUCUCUAAGCUACAUAAUACCAACUUACACUUUACAAAAUGUUGUCCCCCAAAAUGUAGCCAUUCGUAUCUGCUCCUAAUAAAAAGAAAGUUUC |
| | | UUCACAUUCUCUCGAGAAAAAAAAAAAAUGGAAAAAAAAAAAACGGAAAAAAAAAAAAGGUAAAAAAAAAAAAUAUAAAAAAAAAAAACAUAAAAAA |
| | | AAAAAACGAAAAAAAAAAAACGUAAAAAAAAAAAACUCAAAAAAAAAAAAGAUAAAAAAAAAAAACCUAAAAAAAAAAAAUGUAAAAAAAAAAAAGG |
| | | GAAAAAAAAAAAACGCAAAAAAAAAAAACACAAAAAAAAAAAAUGCAAAAAAAAAAAAUCGAAAAAAAAAAAAUCUAAAAAAAAAAAACGAAAAAAA |
| | | AAAAACCCAAAAAAAAAAAAGACAAAAAAAAAAAAUAGAAAAAAAAAAAAGUUAAAAAAAAAAAACUGAAAAAAAAAAAAUUUAAAAAAAAAAAAUC |
| | | UAG |
| mRNA Q | 369 | GGGAAGCUCAGAAUAAACGCUCAACUUUGGCCGGAUCUGCCACCAUGGACGGCUCCGGCGGCGGCUCCCCCAAGAAGAAGCGGAAGGUGGGCGGCUC |
| encoding | | CGGCGGCGGCGCCGCCUUCAAGCCCAACCCCAUCAACUACAUCCUGGGCCUGGACAUCGGCAUCGCCUCCGUGGGCUGGGCCAUGGUGGAGAUCGAC |
| Nme2Cas9 | | GAGGAGGAGAACCCCAUCCGGCUGAUCGACCUGGGCGUGCGGGUGUUCGAGCGGGCCGAGGUGCCCAAGACCGGCGACUCCCUGGCCAUGGCCCGGC |
| | | GGCUGGCCCGGUCCGUGCGGCGGCUGACCCGGCGGCGGGCCCACCGGCUGCUGCGGGCCCGGCGGCUGCUGAAGCGGGAGGGCGUGCUGCAGGCCGC |
| | | CGACUUCGACGAGAACGGCCUGAUCAAGUCCCUGCCCAACACCCCCUGGCAGCUGCGGGCCGCCGCCCUGGACCGGAAGCUGACCCCCCUGGAGUGG |
| | | UCCGCCGUGCUGCUGCACCUGAUCAAGCACCGGGGCUACCUGUCCCAGCGGAAGAACGAGGGCGAGACCGCCGACAAGGAGCUGGGCGCCCUGCUGA |
| | | AGGGCGUGGCCAACAACGCCCACGCCCUGCAGACCGGCGACUUCCGGACCCCCGCCGAGCUGGCCCUGAACAAGUUCGAGAAGGAGUCCGGCCACAU |
| | | CCGGAACCAGCGGGGCGACUACUCCCACACCUUCUCCCGGAAGGACCUGCAGGCCGAGCUGAUCCUGCUGUUCGAGAAGCAGAAGGAGUUCGGCAAC |
| | | CCCCACGUGUCCGGCGGCCUGAAGGAGGGCAUCGAGACCCUGCUGAUGACCCAGCGGCCCGCCCUGUCCGGCGACGCCGUGCAGAAGAUGCUGGGCC |
| | | ACUGCACCUUCGAGCCCGCCGAGCCCAAGGCCGCCAAGAACACCUACACCGCCGAGCGGUUCAUCUGGCUGACCAAGCUGAACAACCUGCGGAUCCU |
| | | GGAGCAGGGCUCCGAGCGGCCCCUGACCGACACCGAGCGGGCCACCCUGAUGGACGAGCCCUACCGGAAGUCCAAGCUGACCUACGCCCAGGCCCGG |
| | | AAGCUGCUGGGCCUGGAGGACACCGCCUUCUUCAAGGGCCUGCGGUACGGCAAGGACAACGCCGAGGCCUCCACCCUGAUGGAGAUGAAGGCCUACC |
| | | ACGCCAUCUCCCGGGCCCUGGAGAAGGAGGGCCUGAAGGACAAGAAGUCCCCCCUGAACCUGUCCUCCGAGCUGCAGGACGAGAUCGGCACCGCCUU |
| | | CUCCCUGUUCAAGACCGACGAGGACAUCACCGGCCGGCUGAAGGACCGGGUGCAGCCCGAGAUCCUGGAGGCCCUGCUGAAGCACAUCUCCUUCGAC |
| | | AAGUUCGUGCAGAUCUCCCUGAAGGCCCUGCGGCGGAUCGUGCCCCUGAUGGAGCAGGGCAAGCGGUACGACGAGGCCUGCGCCGAGAUCUACGGCG |
| | | ACCACUACGGCAAGAAGAACACCGAGGAGAAGAUCUACCUGCCCCCCAUCCCCGCCGACGAGAUCCGGAACCCCGUGGUGCUGCGGGCCCUGUCCCA |
| | | GGCCCGGAAGGUGAUCAACGGCGUGGUGCGGCGGUACGGCUCCCCCGCCCGGAUCCACAUCGAGACCGCCCGGGAGGUGGGCAAGUCCUUCAAGGAC |
| | | CGGAAGGAGAUCGAGAAGCGGCAGGAGGAGAACCGGAAGGACCGGGAGAAGGCCGCCGCCAAGUUCCGGGAGUACUUCCCCAACUUCGUGGGCGAGC |
| | | CCAAGUCCAAGGACAUCCUGAAGCUGCGGCUGUACGAGCAGCAGCACGGCAAGUGCCUGUACUCCGGCAAGGAGAUCAACCUGGUGCGGCUGAACGA |
| | | GAAGGGCUACGUGGAGAUCGACCACGCCCUGCCCUUCUCCCGGACCUGGGACGACUCCUUCAACAACAAGGUGCUGGUGCUGGGCUCCGAGAACCAG |
| | | AACAAGGGCAACCAGACCCCCUACGAGUACUUCAACGGCAAGGACAACUCCCGGGAGUGGCAGGAGUUCAAGGCCCGGGUGGAGACCUCCCGGUUCC |
| | | CCCGGUCCAAGAAGCAGCGGAUCCUGCUGCAGAAGUUCGACGAGGACGGCUUCAAGGAGUGCAACCUGAACGACACCCGGUACGUGAACCGGUUCCU |
| | | GUGCCAGUUCGUGGCCGACCACAUCCUGCUGACCGGCAAGGGCAAGCGGCGGGUGUUCGCCUCCAACGGCCAGAUCACCAACCUGCUGCGGGGCUUC |
| | | UGGGGCCUGCGGAAGGUGCGGGCCGAGAACGACCGGCACCACGCCCUGGACGCCGUGGUGGUGGCCUGCUCCACCGUGGCCAUGCAGCAGAAGAUCA |
| | | CCCGGUUCGUGCGGUACAAGGAGAUGAACGCCUUCGACGGCAAGACCAUCGACAAGGAGACCGGCAAGGUGCUGCACCAGAAGACCCACUUCCCCCA |
| | | GCCCUGGGAGUUCUUCGCCCAGGAGGUGAUGAUCCGGGUGUUCGGCAAGCCCGACGGCAAGCCCGAGUUCGAGGAGGCCGACACCCCCGAGAAGCUG |
| | | CGGACCCUGCUGGCCGAGAAGCUGUCCUCCCGGCCCGAGGCCGUGCACGAGUACGUGACCCCCCUGUUCGUGUCCCGGGCCCCCAACCGGAAGAUGU |
| | | CCGGCGCCCACAAGGACACCCUGCGGUCCGCCAAGCGGUUCGUGAAGCACAACGAGAAGAUCUCCGUGAAGCGGGUGUGGCUGACCGAGAUCAAGCU |
| | | GGCCGACCUGGAGAACAUGGUGAACUACAAGAACGGCCGGGAGAUCGAGCUGUACGAGGCCCUGAAGGCCCGGCUGGAGGCCUACGGCGGCAACGCC |
| | | AAGCAGGCCUUCGACCCCAAGGACAACCCCUUCUACAAGAAGGGCGGCCAGCUGGUGAAGGCCGUGCGGGUGGAGAAGACCCAGGAGUCCGGCGUGC |
| | | UGCUGAACAAGAAGAACGCCUACACCAUCGCCGACAACGGCGACAUGGUGCGGGUGGACGUGUUCUGCAAGGUGGACAAGAAGGGCAAGAACCAGUA |
| | | CUUCAUCGUGCCCAUCUACGCCUGGCAGGUGGCCGAGAACAUCCUGCCCGACAUCGACUGCAAGGGCUACCGGAUCGACGACUCCUACACCUUCUGC |
| | | UUCUCCCUGCACAAGUACGACCUGAUCGCCUUCCAGAAGGACGAGAAGUCCAAGGUGGAGUUCGCCUACUACAUCAACUGCGACUCCUCCAACGGCC |
| | | GGUUCUACCUGGCCUGGCACGACAAGGGCUCCAAGGAGCAGCAGUUCCGGAUCUCCACCCAGAACCUGGUGCUGAUCCAGAAGUACCAGGUGAACGA |
| | | GCUGGGCAAGGAGAUCCGGCCCUGCCGGCUGAAGAAGCGGCCCCCCGUGCGGUAGCUAGCACCAGCCUCAAGAACACCCGAAUGGAGUCUCUAAGCU |
| | | ACAUAAUACCAACUUACACUUUACAAAAUGUUGUCCCCCAAAAUGUAGCCAUUCGUAUCUGCUCCUAAUAAAAAGAAAGUUUCUUCACAUUCUCUCG |
| | | AGAAAAAAAAAAAAUGGAAAAAAAAAAAACGGAAAAAAAAAAAAGGUAAAAAAAAAAAAUAUAAAAAAAAAAAACAUAAAAAAAAAAAACGAAAAAA |
| | | AAAAAACGUAAAAAAAAAAAACUCAAAAAAAAAAAGAUAAAAAAAAAAAACCUAAAAAAAAAAAAUGUAAAAAAAAAAAAGGGAAAAAAAAAAAACG |
| | | CAAAAAAAAAAAACACAAAAAAAAAAAAUGCAAAAAAAAAAAAUCGAAAAAAAAAAAAUCUAAAAAAAAAAAACGAAAAAAAAAAAACCCAAAAAAA |
| | | AAAAAGACAAAAAAAAAAAAUAGAAAAAAAAAAAGUUAAAAAAAAAAAACUGAAAAAAAAAAAAUUUAAAAAAAAAAAAUCUAG |
| mRNA S | 370 | GGGAAGCUCAGAAUAAACGCUCAACUUUGGCCGGAUCUGCCACCAUGGACGGCUCCGGCGGCGGCUCCCCCAAGAAGAAGCGGAAGGUGGAGGACAA |
| encoding | | GCGGCCCGCCGCCACCAAGAAGGCCGGCCAGGCCAAGAAGAAGAAGGGCGGCUCCGGCGGCGGCGAGGCCUCCCCCGCCUCCGGCCCCCGGCACCUG |
| Nme2Cas9 | | AUGGACCCCCACAUCUUCACCUCCAACUUCAACAACGGCAUCGGCCGGCACAAGACCUACCUGUGCUACGAGGUGGAGCGGCUGGACAACGGCACCU |
| base editor | | CCGUGAAGAUGGACCAGCACCGGGGCUUCCUGCACAACCAGGCCAAGAACCUGCUGUGCGGCUUCUACGGCCGGCACGCCGAGCUGCGGUUCCUGGA |
| | | CCUGGUGCCCUCCCUGCAGCUGGACCCCGCCCAGAUCUACCGGGUGACCUGGUUCAUCUCCUGGUCCCCCUGCUUCUCCUGGGGCUGCGCCGGCGAG |
| | | GUGCGGGCCUUCCUGCAGGAGAACACCCACGUGCGGCUGCGGAUCUUCGCCGCCCGGAUCUACGACUACGACCCCCUGUACAAGGAGGCCCUGCAGA |
| | | UGCUGCGGGACGCCGGCGCCCAGGUGUCCAUCAUGACCUACGACGAGUUCAAGCACUGCUGGGACACCUUCGUGGACCACCAGGGCUGCCCCUUCCA |
| | | GCCCUGGGACGGCCUGGACGAGCACUCCCAGGCCCUGUCCGGCCGGCUGCGGGCCAUCCUGCAGAACCAGGGCAACUCCGGCUCCGAGACCCCCGGC |
| | | ACCUCCGAGUCCGCCACCCCCGAGUCCGCAGCGUUCAAACCAAAUCCCAUCAACUACAUCCUGGGCCUGGCCAUCGGCAUCGCCUCCGUGGGCUGGG |
| | | CCAUGGUGGAGAUCGACGAGGAGGAGAACCCCAUCCGGCUGAUCGACCUGGGCGUGCGGGUGUUCGAGCGGGCCGAGGUGCCCAAGACCGGCGACUC |
| | | CCUGGCCAUGGCCCGGCGGCUGGCCCGGUCCGUGCGGCGGCUGACCCGGCGGCGGGCCCACCGGCUGCUGCGGGCCCGGCGGCUGCUGAAGCGGGAG |
| | | GGCGUGCUGCAGGCCGCCGACUUCGACGAGAACGGCCUGAUCAAGUCCCUGCCCAACACCCCCUGGCAGCUGCGGGCCGCCGCCCUGGACCGGAAGC |
| | | UGACCCCCCUGGAGUGGUCCGCCGUGCUGCUGCACCUGAUCAAGCACCGGGGCUACCUGUCCCAGCGGAAGAACGAGGGCGAGACCGCCGACAAGGA |
| | | GCUGGGCGCCCUGCUGAAGGGCGUGGCCAACAACGCCCACGCCCUGCAGACCGGCGACUUCCGGACCCCCGCCGAGCUGGCCCUGAACAAGUUCGAG |
| | | AAGGAGUCCGGCCACAUCCGGAACCAGCGGGGCGACUACUCCCACACCUUCUCCCGGAAGGACCUGCAGGCCGAGCUGAUCCUGCUGUUCGAGAAGC |
| | | AGAAGGAGUUCGGCAACCCCCACGUGUCCGGCGGCCUGAAGGAGGGCAUCGAGACCCUGCUGAUGACCCAGCGGCCCGCCCUGUCCGGCGACGCCGU |
| | | GCAGAAGAUGCUGGGCCACUGCACCUUCGAGCCCGCCGAGCCCAAGGCCGCCAAGAACACCUACACCGCCGAGCGGUUCAUCUGGCUGACCAAGCUG |
| | | AACAACCUGCGGAUCCUGGAGCAGGGCUCCGAGCGGCCCCUGACCGACACCGAGCGGGCCACCCUGAUGGACGAGCCCUACCGGAAGUCCAAGCUGA |
| | | CCUACGCCCAGGCCCGGAAGCUGCUGGGCCUGGAGGACACCGCCUUCUUCAAGGGCCUGCGGUACGGCAAGGACAACGCCGAGGCCUCCACCCUGAU |
| | | GGAGAUGAAGGCCUACCACGCCAUCUCCCGGGCCCUGGAGAAGGAGGGCCUGAAGGACAAGAAGUCCCCCCUGAACCUGUCCUCCGAGCUGCAGGAC |
| | | GAGAUCGGCACCGCCUUCUCCCUGUUCAAGACCGACGAGGACAUCACCGGCCGGCUGAAGGACCGGGUGCAGCCCGAGAUCCUGGAGGCCCUGCUGA |
| | | AGCACAUCUCCUUCGACAAGUUCGUGCAGAUCUCCCUGAAGGCCCUGCGGCGGAUCGUGCCCCUGAUGGAGCAGGGCAAGCGGUACGACGAGGCCUG |
| | | CGCCGAGAUCUACGGCGACCACUACGGCAAGAAGAACACCGAGGAGAAGAUCUACCUGCCCCCCAUCCCCGCCGACGAGAUCCGGAACCCCGUGGUG |
| | | CUGCGGGCCCUGUCCCAGGCCCGGAAGGUGAUCAACGGCGUGGUGCGGCGGUACGGCUCCCCCGCCCGGAUCCACAUCGAGACCGCCCGGGAGGUGG |
| | | GCAAGUCCUUCAAGGACCGGAAGGAGAUCGAGAAGCGGCAGGAGGAGAACCGGAAGGACCGGGAGAAGGCCGCCGCCAAGUUCCGGGAGUACUUCCC |
| | | CAACUUCGUGGGCGAGCCCAAGUCCAAGGACAUCCUGAAGCUGCGGCUGUACGAGCAGCAGCACGGCAAGUGCCUGUACUCCGGCAAGGAGAUCAAC |
| | | CUGGUGCGGCUGAACGAGAAGGGCUACGUGGAGAUCGACCACGCCCUGCCCUUCUCCCGGACCUGGGACGACUCCUUCAACAACAAGGUGCUGGUGC |
| | | UGGGCUCCGAGAACCAGAACAAGGGCAACCAGACCCCCUACGAGUACUUCAACGGCAAGGACAACUCCCGGGAGUGGCAGGAGUUCAAGGCCCGGGU |
| | | GGAGACCUCCCGGUUCCCCCGGUCCAAGAAGCAGCGGAUCCUGCUGCAGAAGUUCGACGAGGACGGCUUCAAGGAGUGCAACCUGAACGACACCCGG |
| | | UACGUGAACCGCUUCCUGUGCCAGUUCGUGGCCGACCACAUCCUGCUGACCGGCAAGGGCAAGCGGCGGGUGUUCGCCUCCAACGGCCAGAUCACCA |
| | | ACCUGCUGCGGGGCUUCUGGGGCCUGCGGAAGGUGCGGGCCGAGAACGACCGGCACCACGCCCUGGACGCCGUGGUGGUGGCCUGCUCCACCGUGGC |
| | | CAUGCAGCAGAAGAUCACCCGGUUCGUGCGGUACAAGGAGAUGAACGCCUUCGACGGCAAGACCAUCGACAAGGAGACCGGCAAGGUGCUGCACCAG |
| | | AAGACCCACUUCCCCCAGCCCUGGGAGUUCUUCGCCCAGGAGGUGAUGAUCCGGGUGUUCGGCAAGCCCGACGGCAAGCCCGAGUUCGAGGAGGCCG |
| | | ACACCCCCGAGAAGCUGCGGACCCUGCUGGCCGAGAAGCUGUCCUCCCGGCCCGAGGCCGUGCACGAGUACGUGACCCCCCUGUUCGUGUCCCGGGC |
| | | CCCCAACCGGAAGAUGUCCGGCGCCCACAAGGACACCCUGCGGUCCGCCAAGCGGUUCGUGAAGCACAACGAGAAGAUCUCCGUGAAGCGGGUGUGG |
| | | CUGACCGAGAUCAAGCUGGCCGACCUGGAGAACAUGGUGAACUACAAGAACGGCCGGGAGAUCGAGCUGUACGAGGCCCUGAAGGCCCGGCUGGAGG |
| | | CCUACGGCGGCAACGCCAAGCAGGCCUUCGACCCCAAGGACAACCCCUUCUACAAGAAGGGCGGCCAGCUGGUGAAGGCCGUGCGGGUGGAGAAGAC |
| | | CCAGGAGUCCGGCGUGCUGCUGAACAAGAAGAACGCCUACACCAUCGCCGACAACGGCGACAUGGUGCGGGUGGACGUGUUCUGCAAGGUGGACAAG |
| | | AAGGGCAAGAACCAGUACUUCAUCGUGCCCAUCUACGCCUGGCAGGUGGCCGAGAACAUCCUGCCCGACAUCGACUGCAAGGGCUACCGGAUCGACG |
| | | ACUCCUACACCUUCUGCUUCUCCCUGCACAAGUACGACCUGAUCGCCUUCCAGAAGGACGAGAAGUCCAAGGUGGAGUUCGCCUACUACAUCAACUG |
| | | CGACUCCUCCAACGGCCGGUUCUACCUGGCCUGGCACGACAAGGGCUCCAAGGAGCAGCAGUUCCGGAUCUCCACCCAGAACCUGGUGCUGAUCCAG |
| | | AAGUACCAGGUGAACGAGCUGGGCAAGGAGAUCCGGCCCUGCCGGCUGAAGAAGCGGCCCCCCGUGCGGUAGUGACUAGCACCAGCCUCAAGAACAC |
| | | CCGAAUGGAGUCUCUAAGCUACAUAAUACCAACUUACACUUUACAAAAUGUUGUCCCCCAAAAUGUAGCCAUUCGUAUCUGCUCCUAAUAAAAAGAA |
| | | AGUUUCUUCACAUUCUCUCGAGAAAAAAAAAAAAUGGAAAAAAAAAAAACGGAAAAAAAAAAAGGUAAAAAAAAAAAAUAUAAAAAAAAAAACAUAA |
| | | AAAAAAAAAACGAAAAAAAAAAAACGUAAAAAAAAAAAACUCAAAAAAAAAAAGAUAAAAAAAAAAAACCUAAAAAAAAAAAAUGUAAAAAAAAAAA |
| | | AGGGAAAAAAAAAAACGCAAAAAAAAAAAACACAAAAAAAAAAAAUGCAAAAAAAAAAAAUCGAAAAAAAAAAAAUCUAAAAAAAAAAAACGAAAAA |
| | | AAAAAAACCCAAAAAAAAAAAAGACAAAAAAAAAAAAUAGAAAAAAAAAAAGUUAAAAAAAAAAAACUGAAAAAAAAAAAAUUUAAAAAAAAAAAAU |
| | | CUAG |
| mRNA E | 371 | GGGAAGCUCAGAAUAAACGCUCAACUUUGGCCGGAUCUGCCACCAUGGAGGCCUCCCCCGCCUCCGGCCCCCGGCACCUGAUGGACCCCCACAUCUU |
| encoding | | CACCUCCAACUUCAACAACGGCAUCGGCCGGCACAAGACCUACCUGUGCUACGAGGUGGAGCGGCUGGACAACGGCACCUCCGUGAAGAUGGACCAG |
| BC22n | | CACCGGGGCUUCCUGCACAACCAGGCCAAGAACCUGCUGUGCGGCUUCUACGGCCGGCACGCCGAGCUGCGGUUCCUGGACCUGGUGCCCUCCCUGC |
| SpyCas9 | | AGCUGGACCCCGCCCAGAUCUACCGGGUGACCUGGUUCAUCUCCUGGUCCCCCUGCUUCUCCUGGGGCUGCGCCGGCGAGGUGCGGGCCUUCCUGCA |
| base editor | | GGAGAACACCCACGUGCGGCUGCGGAUCUUCGCCGCCCGGAUCUACGACUACGACCCCCUGUACAAGGAGGCCCUGCAGAUGCUGCGGGACGCCGGC |
| | | GCCCAGGUGUCCAUCAUGACCUACGACGAGUUCAAGCACUGCUGGGACACCUUCGUGGACCACCAGGGCUGCCCCUUCCAGCCCUGGGACGGCCUGG |
| | | ACGAGCACUCCCAGGCCCUGUCCGGCCGGCUGCGGGCCAUCCUGCAGAACCAGGGCAACUCCGGCUCCGAGACCCCCGGCACCUCCGAGUCCGCCAC |
| | | CCCCGAGUCCGACAAGAAGUACUCCAUCGGCCUGGCCAUCGGCACCAACUCCGUGGGCUGGGCCGUGAUCACCGACGAGUACAAGGUGCCCUCCAAG |
| | | AAGUUCAAGGUGCUGGGCAACACCGACCGGCACUCCAUCAAGAAGAACCUGAUCGGCGCCCUGCUGUUCGACUCCGGCGAGACCGCCGAGGCCACCC |
| | | GGCUGAAGCGGACCGCCCGGCGGCGGUACACCCGGCGGAAGAACCGGAUCUGCUACCUGCAGGAGAUCUUCUCCAACGAGAUGGCCAAGGUGGACGA |
| | | CUCCUUCUUCCACCGGCUGGAGGAGUCCUUCCUGGUGGAGGAGGACAAGAAGCACGAGCGGCACCCCAUCUUCGGCAACAUCGUGGACGAGGUGGCC |
| | | UACCACGAGAAGUACCCCACCAUCUACCACCUGCGGAAGAAGCUGGUGGACUCCACCGACAAGGCCGACCUGCGGCUGAUCUACCUGGCCCUGGCCC |
| | | ACAUGAUCAAGUUCCGGGGCCACUUCCUGAUCGAGGGCGACCUGAACCCCGACAACUCCGACGUGGACAAGCUGUUCAUCCAGCUGGUGCAGACCUA |
| | | CAACCAGCUGUUCGAGGAGAACCCCAUCAACGCCUCCGGCGUGGACGCCAAGGCCAUCCUGUCCGCCCGGCUGUCCAAGUCCCGGCGGCUGGAGAAC |
| | | CUGAUCGCCCAGCUGCCCGGCGAGAAGAAGAACGGCCUGUUCGGCAACCUGAUCGCCCUGUCCCUGGGCCUGACCCCCAACUUCAAGUCCAACUUCG |
| | | ACCUGGCCGAGGACGCCAAGCUGCAGCUGUCCAAGGACACCUACGACGACGACCUGGACAACCUGCUGGCCCAGAUCGGCGACCAGUACGCCGACCU |
| | | GUUCCUGGCCGCCAAGAACCUGUCCGACGCCAUCCUGCUGUCCGACAUCCUGCGGGUGAACACCGAGAUCACCAAGGCCCCCCUGUCCGCCUCCAUG |
| | | AUCAAGCGGUACGACGAGCACCACCAGGACCUGACCCUGCUGAAGGCCCUGGUGCGGCAGCAGCUGCCCGAGAAGUACAAGGAGAUCUUCUUCGACC |
| | | AGUCCAAGAACGGCUACGCCGGCUACAUCGACGGCGGCGCCUCCCAGGAGGAGUUCUACAAGUUCAUCAAGCCCAUCCUGGAGAAGAUGGACGGCAC |
| | | CGAGGAGCUGCUGGUGAAGCUGAACCGGGAGGACCUGCUGCGGAAGCAGCGGACCUUCGACAACGGCUCCAUCCCCCACCAGAUCCACCUGGGCGAG |
| | | CUGCACGCCAUCCUGCGGCGGCAGGAGGACUUCUACCCCUUCCUGAAGGACAACCGGGAGAAGAUCGAGAAGAUCCUGACCUUCCGGAUCCCCUACU |
| | | ACGUGGGCCCCCUGGCCCGGGGCAACUCCCGGUUCGCCUGGAUGACCCGGAAGUCCGAGGAGACCAUCACCCCCUGGAACUUCGAGGAGGUGGUGGA |
| | | CAAGGGCGCCUCCGCCCAGUCCUUCAUCGAGCGGAUGACCAACUUCGACAAGAACCUGCCCAACGAGAAGGUGCUGCCCAAGCACUCCCUGCUGUAC |
| | | GAGUACUUCACCGUGUACAACGAGCUGACCAAGGUGAAGUACGUGACCGAGGGCAUGCGGAAGCCCGCCUUCCUGUCCGGCGAGCAGAAGAAGGCCA |
| | | UCGUGGACCUGCUGUUCAAGACCAACCGGAAGGUGACCGUGAAGCAGCUGAAGGAGGACUACUUCAAGAAGAUCGAGUGCUUCGACUCCGUGGAGAU |
| | | CUCCGGCGUGGAGGACCGGUUCAACGCCUCCCUGGGCACCUACCACGACCUGCUGAAGAUCAUCAAGGACAAGGACUUCCUGGACAACGAGGAGAAC |
| | | GAGGACAUCCUGGAGGACAUCGUGCUGACCCUGACCCUGUUCGAGGACCGGGAGAUGAUCGAGGAGCGGCUGAAGACCUACGCCCACCUGUUCGACG |
| | | ACAAGGUGAUGAAGCAGCUGAAGCGGCGGCGGUACACCGGCUGGGGCCGGCUGUCCCGGAAGCUGAUCAACGGCAUCCGGGACAAGCAGUCCGGCAA |
| | | GACCAUCCUGGACUUCCUGAAGUCCGACGGCUUCGCCAACCGGAACUUCAUGCAGCUGAUCCACGACGACUCCCUGACCUUCAAGGAGGACAUCCAG |
| | | AAGGCCCAGGUGUCCGGCCAGGGCGACUCCCUGCACGAGCACAUCGCCAACCUGGCCGGCUCCCCCGCCAUCAAGAAGGGCAUCCUGCAGACCGUGA |
| | | AGGUGGUGGACGAGCUGGUGAAGGUGAUGGGCCGGCACAAGCCCGAGAACAUCGUGAUCGAGAUGGCCCGGGAGAACCAGACCACCCAGAAGGGCCA |
| | | GAAGAACUCCCGGGAGCGGAUGAAGCGGAUCGAGGAGGGCAUCAAGGAGCUGGGCUCCCAGAUCCUGAAGGAGCACCCCGUGGAGAACACCCAGCUG |
| | | CAGAACGAGAAGCUGUACCUGUACUACCUGCAGAACGGCCGGGACAUGUACGUGGACCAGGAGCUGGACAUCAACCGGCUGUCCGACUACGACGUGG |
| | | ACCACAUCGUGCCCCAGUCCUUCCUGAAGGACGACUCCAUCGACAACAAGGUGCUGACCCGGUCCGACAAGAACCGGGGCAAGUCCGACAACGUGCC |
| | | CUCCGAGGAGGUGGUGAAGAAGAUGAAGAACUACUGGCGGCAGCUGCUGAACGCCAAGCUGAUCACCCAGCGGAAGUUCGACAACCUGACCAAGGCC |
| | | GAGCGGGGCGGCCUGUCCGAGCUGGACAAGGCCGGCUUCAUCAAGCGGCAGCUGGUGGAGACCCGGCAGAUCACCAAGCACGUGGCCCAGAUCCUGG |
| | | ACUCCCGGAUGAACACCAAGUACGACGAGAACGACAAGCUGAUCCGGGAGGUGAAGGUGAUCACCCUGAAGUCCAAGCUGGUGUCCGACUUCCGGAA |
| | | GGACUUCCAGUUCUACAAGGUGCGGGAGAUCAACAACUACCACCACGCCCACGACGCCUACCUGAACGCCGUGGUGGGCACCGCCCUGAUCAAGAAG |
| | | UACCCCAAGCUGGAGUCCGAGUUCGUGUACGGCGACUACAAGGUGUACGACGUGCGGAAGAUGAUCGCCAAGUCCGAGCAGGAGAUCGGCAAGGCCA |
| | | CCGCCAAGUACUUCUUCUACUCCAACAUCAUGAACUUCUUCAAGACCGAGAUCACCCUGGCCAACGGCGAGAUCCGGAAGCGGCCCCUGAUCGAGAC |
| | | CAACGGCGAGACCGGCGAGAUCGUGUGGGACAAGGGCCGGGACUUCGCCACCGUGCGGAAGGUGCUGUCCAUGCCCCAGGUGAACAUCGUGAAGAAG |
| | | ACCGAGGUGCAGACCGGCGGCUUCUCCAAGGAGUCCAUCCUGCCCAAGCGGAACUCCGACAAGCUGAUCGCCCGGAAGAAGGACUGGGACCCCAAGA |
| | | AGUACGGCGGCUUCGACUCCCCCACCGUGGCCUACUCCGUGCUGGUGGUGGCCAAGGUGGAGAAGGGCAAGUCCAAGAAGCUGAAGUCCGUGAAGGA |
| | | GCUGCUGGGCAUCACCAUCAUGGAGCGGUCCUCCUUCGAGAAGAACCCCAUCGACUUCCUGGAGGCCAAGGGCUACAAGGAGGUGAAGAAGGACCUG |
| | | AUCAUCAAGCUGCCCAAGUACUCCCUGUUCGAGCUGGAGAACGGCCGGAAGCGGAUGCUGGCCUCCGCCGGCGAGCUGCAGAAGGGCAACGAGCUGG |
| | | CCCUGCCCUCCAAGUACGUGAACUUCCUGUACCUGGCCUCCCACUACGAGAAGCUGAAGGGCUCCCCCGAGGACAACGAGCAGAAGCAGCUGUUCGU |
| | | GGAGCAGCACAAGCACUACCUGGACGAGAUCAUCGAGCAGAUCUCCGAGUUCUCCAAGCGGGUGAUCCUGGCCGACGCCAACCUGGACAAGGUGCUG |
| | | UCCGCCUACAACAAGCACCGGGACAAGCCCAUCCGGGAGCAGGCCGAGAACAUCAUCCACCUGUUCACCCUGACCAACCUGGGCGCCCCCGCCGCCU |
| | | UCAAGUACUUCGACACCACCAUCGACCGGAAGCGGUACACCUCCACCAAGGAGGUGCUGGACGCCACCCUGAUCCACCAGUCCAUCACCGGCCUGUA |
| | | CGAGACCCGGAUCGACCUGUCCCAGCUGGGCGGCGACGGCGGCGGCUCCCCCAAGAAGAAGCGGAAGGUGUGACUAGCACCAGCCUCAAGAACACCC |
| | | GAAUGGAGUCUCUAAGCUACAUAAUACCAACUUACACUUUACAAAAUGUUGUCCCCCAAAAUGUAGCCAUUCGUAUCUGCUCCUAAUAAAAAGAAAG |
| | | UUUCUUCACAUUCUCUCGAGAAAAAAAAAAAAUGGAAAAAAAAAAAACGGAAAAAAAAAAAAGGUAAAAAAAAAAAAUAUAAAAAAAAAAAACAUAA |
| | | AAAAAAAAAACGAAAAAAAAAAAACGUAAAAAAAAAAAACUCAAAAAAAAAAAAGAUAAAAAAAAAAAACCUAAAAAAAAAAAAUGUAAAAAAAAAA |
| | | AAGGGAAAAAAAAAAAACGCAAAAAAAAAAAACACAAAAAAAAAAAAUGCAAAAAAAAAAAAUCGAAAAAAAAAAAAUCUAAAAAAAAAAAACGAAA |
| | | AAAAAAAAACCCAAAAAAAAAAAAGACAAAAAAAAAAAAUAGAAAAAAAAAAAAGUUAAAAAAAAAAAACUGAAAAAAAAAAAAUUUAAAAAAAAAA |
| | | AAUCUAG |
| Open | 372 | atgaccggtgccgccttcaagcccaaccccatcaactacatcctgggcctggacatcggcatcgcctccgtgggctgggccatggtggagatcgacg |
| reading | | aggaggagaaccccatccggctgatcgacctgggcgtgcgggtgttcgagcgggccgaggtgcccaagaccggcgactccctggccatggcccggcg |
| frame for | | gctggcccggtccgtgcggcggctgacccggcggcgggcccaccggctgctgcgggcccggcggctgctgaagcgggagggcgtgctgcaggccgcc |
| Nme2Cas9 | | gacttcgacgagaacggcctgatcaagtccctgcccaacaccccctggcagctgcgggccgccgccctggaccggaagctgacccccctggagtggt |
| encoded by | | ccgccgtgctgctgcacctgatcaagcaccggggctacctgtcccagcggaagaacgagggcgagaccgccgacaaggagctgggcgccctgctgaa |
| mRNA C | | gggcgtggccaacaacgcccacgccctgcagaccggcgacttccggacccccgccgagctggccctgaacaagttcgagaaggagtccggccacatc |
| | | cggaaccagcggggcgactactcccacaccttctcccggaaggacctgcaggccgagctgatcctgctgttcgagaagcagaaggagttcggcaacc |
| | | cccacgtgtccggcggcctgaaggagggcatcgagaccctgctgatgacccagcggcccgccctgtccggcgacgccgtgcagaagatgctgggcca |
| | | ctgcaccttcgagcccgccgagcccaaggccgccaagaacacctacaccgccgagcggttcatctggctgaccaagctgaacaacctgcggatcctg |
| | | gagcagggctccgagcggcccctgaccgacaccgagcgggccaccctgatggacgagccctaccggaagtccaagctgacctacgcccaggcccgga |
| | | agctgctgggcctggaggacaccgccttcttcaagggcctgcggtacggcaaggacaacgccgaggcctccaccctgatggagatgaaggcctacca |
| | | cgccatctcccgggccctggagaaggagggcctgaaggacaagaagtcccccctgaacctgtcctccgagctgcaggacgagatcggcaccgccttc |
| | | tccctgttcaagaccgacgaggacatcaccggccggctgaaggaccgggtgcagcccgagatcctggaggccctgctgaagcacatctccttcgaca |
| | | agttcgtgcagatctccctgaaggccctgcggcggatcgtgcccctgatggagcagggcaagcggtacgacgaggcctgcgccgagatctacggcga |
| | | ccactacggcaagaagaacaccgaggagaagatctacctgccccccatccccgccgacgagatccggaaccccgtggtgctgcgggccctgtcccag |
| | | gcccggaaggtgatcaacggcgtggtgcggcggtacggctcccccgcccggatccacatcgagaccgcccgggaggtgggcaagtccttcaaggacc |
| | | ggaaggagatcgagaagcggcaggaggagaaccggaaggaccgggagaaggccgccgccaagttccgggagtacttccccaacttcgtgggcgagcc |
| | | caagtccaaggacatcctgaagctgcggctgtacgagcagcagcacggcaagtgcctgtactccggcaaggagatcaacctggtgcggctgaacgag |
| | | aagggctacgtggagatcgaccacgccctgcccttctcccggacctgggacgactccttcaacaacaaggtgctggtgctgggctccgagaaccaga |
| | | acaagggcaaccagaccccctacgagtacttcaacggcaaggacaactcccgggagtggcaggagttcaaggcccgggtggagacctcccggttccc |
| | | ccggtccaagaagcagcggatcctgctgcagaagttcgacgaggacggcttcaaggagtgcaacctgaacgacacccggtacgtgaaccgcttcctg |
| | | tgccagttcgtggccgaccacatcctgctgaccggcaagggcaagcggcgggtgttcgcctccaacggccagatcaccaacctgctgcggggcttct |
| | | ggggcctgcggaaggtgcgggccgagaacgaccggcaccacgccctggacgccgtggtggtggcctgctccaccgtggccatgcagcagaagatcac |
| | | ccggttcgtgcggtacaaggagatgaacgccttcgacggcaagaccatcgacaaggagaccggcaaggtgctgcaccagaagacccacttcccccag |
| | | ccctgggagttcttcgcccaggaggtgatgatccgggtgttcggcaagcccgacggcaagcccgagttcgaggaggccgacacccccgagaagctgc |
| | | ggaccctgctggccgagaagctgtcctcccggcccgaggccgtgcacgagtacgtgacccccctgttcgtgtcccgggcccccaaccggaagatgtc |
| | | cggcgcccacaaggacaccctgcggtccgccaagcggttcgtgaagcacaacgagaagatctccgtgaagcgggtgtggctgaccgagatcaagctg |
| | | gccgacctggagaacatggtgaactacaagaacggccgggagatcgagctgtacgaggccctgaaggcccggctggaggcctacggcggcaacgcca |
| | | agcaggccttcgaccccaaggacaaccccttctacaagaagggcggccagctggtgaaggccgtgcgggtggagaagacccaggagtccggcgtgct |
| | | gctgaacaagaagaacgcctacaccatcgccgacaacggcgacatggtgcgggtggacgtgttctgcaaggtggacaagaagggcaagaaccagtac |
| | | ttcatcgtgcccatctacgcctggcaggtggccgagaacatcctgcccgacatcgactgcaagggctaccggatcgacgactcctacaccttctgct |
| | | tctccctgcacaagtacgacctgatcgccttccagaaggacgagaagtccaaggtggagttcgcctactacatcaactgcgactcctccaacggccg |
| | | gttctacctggcctggcacgacaagggctccaaggagcagcagttccggatctccacccagaacctggtgctgatccagaagtaccaggtgaacgag |
| | | ctgggcaaggagatccggccctgccggctgaagaagcggccccccgtgcggtccggaaagcggaccgccgacggctccgagttcgagtcccccaaga |
| | | agaagcggaaggtggagtag |
| Open | 373 | ATGACCAACCTGTCCGACATCATCGAGAAGGAGACCGGCAAGCAGCTGGTGATCCAGGAGTCCATCCTGATGCTGCCCGAGGAGGTGGAGGAGGTGA |
| reading | | TCGGCAACAAGCCCGAGTCCGACATCCTGGTGCACACCGCCTACGACGAGTCCACCGACGAGAACGTGATGCTGCTGACCTCCGACGCCCCCGAGTA |
| frame for | | CAAGCCCTGGGCCCTGGTGATCCAGGACTCCAACGGCGAGAACAAGATCAAGATGCTGTCCGGCGGCTCCAAGCGGACCGCCGACGGCTCCGAGTTC |
| UGI encoded | | GAGTCCCCCAAGAAGAAGCGGAAGGTGGAGTGATAG |
| by mRNA G | | |
| Open | 374 | ATGGTGCCCAAGAAGAAGCGGAAGGTGGCCGCCTTCAAGCCCAACCCCATCAACTACATCCTGGGCCTGGACATCGGCATCGCCTCCGTGGGCTGGG |
| reading | | CCATGGTGGAGATCGACGAGGAGGAGAACCCCATCCGGCTGATCGACCTGGGCGTGCGGGTGTTCGAGCGGGCCGAGGTGCCCAAGACCGGCGACTC |
| frame for | | CCTGGCCATGGCCCGGCGGCTGGCCCGGTCCGTGCGGCGGCTGACCCGGCGGCGGGCCCACCGGCTGCTGCGGGCCCGGCGGCTGCTGAAGCGGGAG |
| Nme2Cas9 | | GGCGTGCTGCAGGCCGCCGACTTCGACGAGAACGGCCTGATCAAGTCCCTGCCCAACACCCCCTGGCAGCTGCGGGCCGCCGCCCTGGACCGGAAGC |
| encoded by | | TGACCCCCCTGGAGTGGTCCGCCGTGCTGCTGCACCTGATCAAGCACCGGGGCTACCTGTCCCAGCGGAAGAACGAGGGCGAGACCGCCGACAAGGA |
| mRNA I | | GCTGGGCGCCCTGCTGAAGGGCGTGGCCAACAACGCCCACGCCCTGCAGACCGGCGACTTCCGGACCCCCGCCGAGCTGGCCCTGAACAAGTTCGAG |
| | | AAGGAGTCCGGCCACATCCGGAACCAGCGGGGCGACTACTCCCACACCTTCTCCCGGAAGGACCTGCAGGCCGAGCTGATCCTGCTGTTCGAGAAGC |
| | | AGAAGGAGTTCGGCAACCCCCACGTGTCCGGCGGCCTGAAGGAGGGCATCGAGACCCTGCTGATGACCCAGCGGCCCGCCCTGTCCGGCGACGCCGT |
| | | GCAGAAGATGCTGGGCCACTGCACCTTCGAGCCCGCCGAGCCCAAGGCCGCCAAGAACACCTACACCGCCGAGCGGTTCATCTGGCTGACCAAGCTG |
| | | AACAACCTGCGGATCCTGGAGCAGGGCTCCGAGCGGCCCCTGACCGACACCGAGCGGGCCACCCTGATGGACGAGCCCTACCGGAAGTCCAAGCTGA |
| | | CCTACGCCCAGGCCCGGAAGCTGCTGGGCCTGGAGGACACCGCCTTCTTCAAGGGCCTGCGGTACGGCAAGGACAACGCCGAGGCCTCCACCCTGAT |
| | | GGAGATGAAGGCCTACCACGCCATCTCCCGGGCCCTGGAGAAGGAGGGCCTGAAGGACAAGAAGTCCCCCCTGAACCTGTCCTCCGAGCTGCAGGAC |
| | | GAGATCGGCACCGCCTTCTCCCTGTTCAAGACCGACGAGGACATCACCGGCCGGCTGAAGGACCGGGTGCAGCCCGAGATCCTGGAGGCCCTGCTGA |
| | | AGCACATCTCCTTCGACAAGTTCGTGCAGATCTCCCTGAAGGCCCTGCGGCGGATCGTGCCCCTGATGGAGCAGGGCAAGCGGTACGACGAGGCCTG |
| | | CGCCGAGATCTACGGCGACCACTACGGCAAGAAGAACACCGAGGAGAAGATCTACCTGCCCCCCATCCCCGCCGACGAGATCCGGAACCCCGTGGTG |
| | | CTGCGGGCCCTGTCCCAGGCCCGGAAGGTGATCAACGGCGTGGTGCGGCGGTACGGCTCCCCCGCCCGGATCCACATCGAGACCGCCCGGGAGGTGG |
| | | GCAAGTCCTTCAAGGACCGGAAGGAGATCGAGAAGCGGCAGGAGGAGAACCGGAAGGACCGGGAGAAGGCCGCCGCCAAGTTCCGGGAGTACTTCCC |
| | | CAACTTCGTGGGCGAGCCCAAGTCCAAGGACATCCTGAAGCTGCGGCTGTACGAGCAGCAGCACGGCAAGTGCCTGTACTCCGGCAAGGAGATCAAC |
| | | CTGGTGCGGCTGAACGAGAAGGGCTACGTGGAGATCGACCACGCCCTGCCCTTCTCCCGGACCTGGGACGACTCCTTCAACAACAAGGTGCTGGTGC |
| | | TGGGCTCCGAGAACCAGAACAAGGGCAACCAGACCCCCTACGAGTACTTCAACGGCAAGGACAACTCCCGGGAGTGGCAGGAGTTCAAGGCCCGGGT |
| | | GGAGACCTCCCGGTTCCCCCGGTCCAAGAAGCAGCGGATCCTGCTGCAGAAGTTCGACGAGGACGGCTTCAAGGAGTGCAACCTGAACGACACCCGG |
| | | TACGTGAACCGGTTCCTGTGCCAGTTCGTGGCCGACCACATCCTGCTGACCGGCAAGGGCAAGCGGCGGGTGTTCGCCTCCAACGGCCAGATCACCA |
| | | ACCTGCTGCGGGGCTTCTGGGGCCTGCGGAAGGTGCGGGCCGAGAACGACCGGCACCACGCCCTGGACGCCGTGGTGGTGGCCTGCTCCACCGTGGC |
| | | CATGCAGCAGAAGATCACCCGGTTCGTGCGGTACAAGGAGATGAACGCCTTCGACGGCAAGACCATCGACAAGGAGACCGGCAAGGTGCTGCACCAG |
| | | AAGACCCACTTCCCCCAGCCCTGGGAGTTCTTCGCCCAGGAGGTGATGATCCGGGTGTTCGGCAAGCCCGACGGCAAGCCCGAGTTCGAGGAGGCCG |
| | | ACACCCCCGAGAAGCTGCGGACCCTGCTGGCCGAGAAGCTGTCCTCCCGGCCCGAGGCCGTGCACGAGTACGTGACCCCCCTGTTCGTGTCCCGGGC |
| | | CCCCAACCGGAAGATGTCCGGCGCCCACAAGGACACCCTGCGGTCCGCCAAGCGGTTCGTGAAGCACAACGAGAAGATCTCCGTGAAGCGGGTGTGG |
| | | CTGACCGAGATCAAGCTGGCCGACCTGGAGAACATGGTGAACTACAAGAACGGCCGGGAGATCGAGCTGTACGAGGCCCTGAAGGCCCGGCTGGAGG |
| | | CCTACGGCGGCAACGCCAAGCAGGCCTTCGACCCCAAGGACAACCCCTTCTACAAGAAGGGCGGCCAGCTGGTGAAGGCCGTGCGGGTGGAGAAGAC |
| | | CCAGGAGTCCGGCGTGCTGCTGAACAAGAAGAACGCCTACACCATCGCCGACAACGGCGACATGGTGCGGGTGGACGTGTTCTGCAAGGTGGACAAG |
| | | AAGGGCAAGAACCAGTACTTCATCGTGCCCATCTACGCCTGGCAGGTGGCCGAGAACATCCTGCCCGACATCGACTGCAAGGGCTACCGGATCGACG |
| | | ACTCCTACACCTTCTGCTTCTCCCTGCACAAGTACGACCTGATCGCCTTCCAGAAGGACGAGAAGTCCAAGGTGGAGTTCGCCTACTACATCAACTG |
| | | CGACTCCTCCAACGGCCGGTTCTACCTGGCCTGGCACGACAAGGGCTCCAAGGAGCAGCAGTTCCGGATCTCCACCCAGAACCTGGTGCTGATCCAG |
| | | AAGTACCAGGTGAACGAGCTGGGCAAGGAGATCCGGCCCTGCCGGCTGAAGAAGCGGCCCCCCGTGCGGTACCCCTACGACGTGCCCGACTACGCCG |
| | | CCGCCCCCGCCGCCAAGAAGAAGAAGCTGGACTAG |
| Open | 375 | ATGGCCGCCTTCAAGCCCAACCCCATCAACTACATCCTGGGCCTGGACATCGGCATCGCCTCCGTGGGCTGGGCCATGGTGGAGATCGACGAGGAGG |
| reading | | AGAACCCCATCCGGCTGATCGACCTGGGCGTGCGGGTGTTCGAGCGGGCCGAGGTGCCCAAGACCGGCGACTCCCTGGCCATGGCCCGGCGGCTGGC |
| frame for | | CCGGTCCGTGCGGCGGCTGACCCGGCGGCGGGCCCACCGGCTGCTGCGGGCCCGGCGGCTGCTGAAGCGGGAGGGCGTGCTGCAGGCCGCCGACTTC |
| Nme2Cas9 | | GACGAGAACGGCCTGATCAAGTCCCTGCCCAACACCCCCTGGCAGCTGCGGGCCGCCGCCCTGGACCGGAAGCTGACCCCCCTGGAGTGGTCCGCCG |
| encoded by | | TGCTGCTGCACCTGATCAAGCACCGGGGCTACCTGTCCCAGCGGAAGAACGAGGGCGAGACCGCCGACAAGGAGCTGGGCGCCCTGCTGAAGGGCGT |
| mRNA J | | GGCCAACAACGCCCACGCCCTGCAGACCGGCGACTTCCGGACCCCCGCCGAGCTGGCCCTGAACAAGTTCGAGAAGGAGTCCGGCCACATCCGGAAC |
| | | CAGCGGGGCGACTACTCCCACACCTTCTCCCGGAAGGACCTGCAGGCCGAGCTGATCCTGCTGTTCGAGAAGCAGAAGGAGTTCGGCAACCCCCACG |
| | | TGTCCGGCGGCCTGAAGGAGGGCATCGAGACCCTGCTGATGACCCAGCGGCCCGCCCTGTCCGGCGACGCCGTGCAGAAGATGCTGGGCCACTGCAC |
| | | CTTCGAGCCCGCCGAGCCCAAGGCCGCCAAGAACACCTACACCGCCGAGCGGTTCATCTGGCTGACCAAGCTGAACAACCTGCGGATCCTGGAGCAG |
| | | GGCTCCGAGCGGCCCCTGACCGACACCGAGCGGGCCACCCTGATGGACGAGCCCTACCGGAAGTCCAAGCTGACCTACGCCCAGGCCCGGAAGCTGC |
| | | TGGGCCTGGAGGACACCGCCTTCTTCAAGGGCCTGCGGTACGGCAAGGACAACGCCGAGGCCTCCACCCTGATGGAGATGAAGGCCTACCACGCCAT |
| | | CTCCCGGGCCCTGGAGAAGGAGGGCCTGAAGGACAAGAAGTCCCCCCTGAACCTGTCCTCCGAGCTGCAGGACGAGATCGGCACCGCCTTCTCCCTG |
| | | TTCAAGACCGACGAGGACATCACCGGCCGGCTGAAGGACCGGGTGCAGCCCGAGATCCTGGAGGCCCTGCTGAAGCACATCTCCTTCGACAAGTTCG |
| | | TGCAGATCTCCCTGAAGGCCCTGCGGCGGATCGTGCCCCTGATGGAGCAGGGCAAGCGGTACGACGAGGCCTGCGCCGAGATCTACGGCGACCACTA |
| | | CGGCAAGAAGAACACCGAGGAGAAGATCTACCTGCCCCCCATCCCCGCCGACGAGATCCGGAACCCCGTGGTGCTGCGGGCCCTGTCCCAGGCCCGG |
| | | AAGGTGATCAACGGCGTGGTGCGGCGGTACGGCTCCCCCGCCCGGATCCACATCGAGACCGCCCGGGAGGTGGGCAAGTCCTTCAAGGACCGGAAGG |
| | | AGATCGAGAAGCGGCAGGAGGAGAACCGGAAGGACCGGGAGAAGGCCGCCGCCAAGTTCCGGGAGTACTTCCCCAACTTCGTGGGCGAGCCCAAGTC |
| | | CAAGGACATCCTGAAGCTGCGGCTGTACGAGCAGCAGCACGGCAAGTGCCTGTACTCCGGCAAGGAGATCAACCTGGTGCGGCTGAACGAGAAGGGC |
| | | TACGTGGAGATCGACCACGCCCTGCCCTTCTCCCGGACCTGGGACGACTCCTTCAACAACAAGGTGCTGGTGCTGGGCTCCGAGAACCAGAACAAGG |
| | | GCAACCAGACCCCCTACGAGTACTTCAACGGCAAGGACAACTCCCGGGAGTGGCAGGAGTTCAAGGCCCGGGTGGAGACCTCCCGGTTCCCCCGGTC |
| | | CAAGAAGCAGCGGATCCTGCTGCAGAAGTTCGACGAGGACGGCTTCAAGGAGTGCAACCTGAACGACACCCGGTACGTGAACCGGTTCCTGTGCCAG |
| | | TTCGTGGCCGACCACATCCTGCTGACCGGCAAGGGCAAGCGGCGGGTGTTCGCCTCCAACGGCCAGATCACCAACCTGCTGCGGGGCTTCTGGGGCC |
| | | TGCGGAAGGTGCGGGCCGAGAACGACCGGCACCACGCCCTGGACGCCGTGGTGGTGGCCTGCTCCACCGTGGCCATGCAGCAGAAGATCACCCGGTT |
| | | CGTGCGGTACAAGGAGATGAACGCCTTCGACGGCAAGACCATCGACAAGGAGACCGGCAAGGTGCTGCACCAGAAGACCCACTTCCCCCAGCCCTGG |
| | | GAGTTCTTCGCCCAGGAGGTGATGATCCGGGTGTTCGGCAAGCCCGACGGCAAGCCCGAGTTCGAGGAGGCCGACACCCCCGAGAAGCTGCGGACCC |
| | | TGCTGGCCGAGAAGCTGTCCTCCCGGCCCGAGGCCGTGCACGAGTACGTGACCCCCCTGTTCGTGTCCCGGGCCCCCAACCGGAAGATGTCCGGCGC |
| | | CCACAAGGACACCCTGCGGTCCGCCAAGCGGTTCGTGAAGCACAACGAGAAGATCTCCGTGAAGCGGGTGTGGCTGACCGAGATCAAGCTGGCCGAC |
| | | CTGGAGAACATGGTGAACTACAAGAACGGCCGGGAGATCGAGCTGTACGAGGCCCTGAAGGCCCGGCTGGAGGCCTACGGCGGCAACGCCAAGCAGG |
| | | CCTTCGACCCCAAGGACAACCCCTTCTACAAGAAGGGCGGCCAGCTGGTGAAGGCCGTGCGGGTGGAGAAGACCCAGGAGTCCGGCGTGCTGCTGAA |
| | | CAAGAAGAACGCCTACACCATCGCCGACAACGGCGACATGGTGCGGGTGGACGTGTTCTGCAAGGTGGACAAGAAGGGCAAGAACCAGTACTTCATC |
| | | GTGCCCATCTACGCCTGGCAGGTGGCCGAGAACATCCTGCCCGACATCGACTGCAAGGGCTACCGGATCGACGACTCCTACACCTTCTGCTTCTCCC |
| | | TGCACAAGTACGACCTGATCGCCTTCCAGAAGGACGAGAAGTCCAAGGTGGAGTTCGCCTACTACATCAACTGCGACTCCTCCAACGGCCGGTTCTA |
| | | CCTGGCCTGGCACGACAAGGGCTCCAAGGAGCAGCAGTTCCGGATCTCCACCCAGAACCTGGTGCTGATCCAGAAGTACCAGGTGAACGAGCTGGGC |
| | | AAGGAGATCCGGCCCTGCCGGCTGAAGAAGCGGCCCCCCGTGCGG |
| Open | 376 | ATGGACGGCTCCGGCGGCGGCTCCCCCAAGAAGAAGCGGAAGGTGGGCGGCTCCGGOGGCGGCGCCGCCTTCAAGCCCAACCCCATCAACTACATCC |
| reading | | TGGGCCTGGACATCGGCATCGCCTCCGTGGGCTGGGCCATGGTGGAGATCGACGAGGAGGAGAACCCCATCCGGCTGATCGACCTGGGCGTGCGGGT |
| frame for | | GTTCGAGCGGGCCGAGGTGCCCAAGACCGGCGACTCCCTGGCCATGGCCCGGCGGCTGGCCCGGTCCGTGCGGCGGCTGACCCGGCGGCGGGCCCAC |
| Nme2Cas9 | | CGGCTGCTGCGGGCCCGGCGGCTGCTGAAGCGGGAGGGCGTGCTGCAGGCCGCCGACTTCGACGAGAACGGCCTGATCAAGTCCCTGCCCAACACCC |
| encoded by | | CCTGGCAGCTGCGGGCCGCCGCCCTGGACCGGAAGCTGACCCCCCTGGAGTGGTCCGCCGTGCTGCTGCACCTGATCAAGCACCGGGGCTACCTGTC |
| mRNA M | | CCAGCGGAAGAACGAGGGCGAGACCGCCGACAAGGAGCTGGGCGCCCTGCTGAAGGGCGTGGCCAACAACGCCCACGCCCTGCAGACCGGCGACTTC |
| | | CGGACCCCCGCCGAGCTGGCCCTGAACAAGTTCGAGAAGGAGTCCGGCCACATCCGGAACCAGCGGGGCGACTACTCCCACACCTTCTCCCGGAAGG |
| | | ACCTGCAGGCCGAGCTGATCCTGCTGTTCGAGAAGCAGAAGGAGTTCGGCAACCCCCACGTGTCCGGCGGCCTGAAGGAGGGCATCGAGACCCTGCT |
| | | GATGACCCAGCGGCCCGCCCTGTCCGGCGACGCCGTGCAGAAGATGCTGGGCCACTGCACCTTCGAGCCCGCCGAGCCCAAGGCCGCCAAGAACACC |
| | | TACACCGCCGAGCGGTTCATCTGGCTGACCAAGCTGAACAACCTGCGGATCCTGGAGCAGGGCTCCGAGCGGCCCCTGACCGACACCGAGCGGGCCA |
| | | CCCTGATGGACGAGCCCTACCGGAAGTCCAAGCTGACCTACGCCCAGGCCCGGAAGCTGCTGGGCCTGGAGGACACCGCCTTCTTCAAGGGCCTGCG |
| | | GTACGGCAAGGACAACGCCGAGGCCTCCACCCTGATGGAGATGAAGGCCTACCACGCCATCTCCCGGGCCCTGGAGAAGGAGGGCCTGAAGGACAAG |
| | | AAGTCCCCCCTGAACCTGTCCTCCGAGCTGCAGGACGAGATCGGCACCGCCTTCTCCCTGTTCAAGACCGACGAGGACATCACCGGCCGGCTGAAGG |
| | | ACCGGGTGCAGCCCGAGATCCTGGAGGCCCTGCTGAAGCACATCTCCTTCGACAAGTTCGTGCAGATCTCCCTGAAGGCCCTGCGGCGGATCGTGCC |
| | | CCTGATGGAGCAGGGCAAGCGGTACGACGAGGCCTGCGCCGAGATCTACGGCGACCACTACGGCAAGAAGAACACCGAGGAGAAGATCTACCTGCCC |
| | | CCCATCCCCGCCGACGAGATCCGGAACCCCGTGGTGCTGCGGGCCCTGTCCCAGGCCCGGAAGGTGATCAACGGCGTGGTGCGGCGGTACGGCTCCC |
| | | CCGCCCGGATCCACATCGAGACCGCCCGGGAGGTGGGCAAGTCCTTCAAGGACCGGAAGGAGATCGAGAAGCGGCAGGAGGAGAACCGGAAGGACCG |
| | | GGAGAAGGCCGCCGCCAAGTTCCGGGAGTACTTCCCCAACTTCGTGGGCGAGCCCAAGTCCAAGGACATCCTGAAGCTGCGGCTGTACGAGCAGCAG |
| | | CACGGCAAGTGCCTGTACTCCGGCAAGGAGATCAACCTGGTGCGGCTGAACGAGAAGGGCTACGTGGAGATCGACCACGCCCTGCCCTTCTCCCGGA |
| | | CCTGGGACGACTCCTTCAACAACAAGGTGCTGGTGCTGGGCTCCGAGAACCAGAACAAGGGCAACCAGACCCCCTACGAGTACTTCAACGGCAAGGA |
| | | CAACTCCCGGGAGTGGCAGGAGTTCAAGGCCCGGGTGGAGACCTCCCGGTTCCCCCGGTCCAAGAAGCAGCGGATCCTGCTGCAGAAGTTCGACGAG |
| | | GACGGCTTCAAGGAGTGCAACCTGAACGACACCCGGTACGTGAACCGGTTCCTGTGCCAGTTCGTGGCCGACCACATCCTGCTGACCGGCAAGGGCA |
| | | AGCGGCGGGTGTTCGCCTCCAACGGCCAGATCACCAACCTGCTGCGGGGCTTCTGGGGCCTGCGGAAGGTGCGGGCCGAGAACGACCGGCACCACGC |
| | | CCTGGACGCCGTGGTGGTGGCCTGCTCCACCGTGGCCATGCAGCAGAAGATCACCCGGTTCGTGCGGTACAAGGAGATGAACGCCTTCGACGGCAAG |
| | | ACCATCGACAAGGAGACCGGCAAGGTGCTGCACCAGAAGACCCACTTCCCCCAGCCCTGGGAGTTCTTCGCCCAGGAGGTGATGATCCGGGTGTTCG |
| | | GCAAGCCCGACGGCAAGCCCGAGTTCGAGGAGGCCGACACCCCCGAGAAGCTGCGGACCCTGCTGGCCGAGAAGCTGTCCTCCCGGCCCGAGGCCGT |
| | | GCACGAGTACGTGACCCCCCTGTTCGTGTCCCGGGCCCCCAACCGGAAGATGTCCGGCGCCCACAAGGACACCCTGCGGTCCGCCAAGCGGTTCGTG |
| | | AAGCACAACGAGAAGATCTCCGTGAAGCGGGTGTGGCTGACCGAGATCAAGCTGGCCGACCTGGAGAACATGGTGAACTACAAGAACGGCCGGGAGA |
| | | TCGAGCTGTACGAGGCCCTGAAGGCCCGGCTGGAGGCCTACGGCGGCAACGCCAAGCAGGCCTTCGACCCCAAGGACAACCCCTTCTACAAGAAGGG |
| | | CGGCCAGCTGGTGAAGGCCGTGCGGGTGGAGAAGACCCAGGAGTCCGGCGTGCTGCTGAACAAGAAGAACGCCTACACCATCGCCGACAACGGCGAC |
| | | ATGGTGCGGGTGGACGTGTTCTGCAAGGTGGACAAGAAGGGCAAGAACCAGTACTTCATCGTGCCCATCTACGCCTGGCAGGTGGCCGAGAACATCC |
| | | TGCCCGACATCGACTGCAAGGGCTACCGGATCGACGACTCCTACACCTTCTGCTTCTCCCTGCACAAGTACGACCTGATCGCCTTCCAGAAGGACGA |
| | | GAAGTCCAAGGTGGAGTTCGCCTACTACATCAACTGCGACTCCTCCAACGGCCGGTTCTACCTGGCCTGGCACGACAAGGGCTCCAAGGAGCAGCAG |
| | | TTCCGGATCTCCACCCAGAACCTGGTGCTGATCCAGAAGTACCAGGTGAACGAGCTGGGCAAGGAGATCCGGCCCTGCCGGCTGAAGAAGCGGCCCC |
| | | CCGTGCGGTCCGAGTCCGCCACCCCCGAGTCCGTGTCCGGCTGGCGGCTGTTCAAGAAGATCTCCTAG |
| Open | 377 | atgGACGGCTCCGGCGGCGGCTCCCCCAAGAAGAAGCGGAAGGTGGGCGGCTCCGGCGGCGGCgccgccttcaagcccaaccccatcaactacatcc |
| reading | | tgggcctggacatcggcatcgcctccgtgggctgggccatggtggagatcgacgaggaggagaaccccatccggctgatcgacctgggcgtgcgggt |
| frame for | | gttcgagcgggccgaggtgcccaagaccggcgactccctggccatggcccggcggctggcccggtccgtgcggcggctgacccggcggcgggcccac |
| Nme2Cas9 | | cggctgctgcgggcccggcggctgctgaagcgggagggcgtgctgcaggccgccgacttcgacgagaacggcctgatcaagtccctgcccaacaccc |
| encoded by | | cctggcagctgcgggccgccgccctggaccggaagctgacccccctggagtggtccgccgtgctgctgcacctgatcaagcaccggggctacctgtc |
| mRNA N | | ccagcggaagaacgagggcgagaccgccgacaaggagctgggcgccctgctgaagggcgtggccaacaacgcccacgccctgcagaccggcgacttc |
| | | cggacccccgccgagctggccctgaacaagttcgagaaggagtccggccacatccggaaccagcggggcgactactcccacaccttctcccggaagg |
| | | acctgcaggccgagctgatcctgctgttcgagaagcagaaggagttcggcaacccccacgtgtccggcggcctgaaggagggcatcgagaccctgct |
| | | gatgacccagcggcccgccctgtccggcgacgccgtgcagaagatgctgggccactgcaccttcgagcccgccgagcccaaggccgccaagaacacc |
| | | tacaccgccgagcggttcatctggctgaccaagctgaacaacctgcggatcctggagcagggctccgagcggcccctgaccgacaccgagcgggcca |
| | | ccctgatggacgagccctaccggaagtccaagctgacctacgcccaggcccggaagctgctgggcctggaggacaccgccttcttcaagggcctgcg |
| | | gtacggcaaggacaacgccgaggcctccaccctgatggagatgaaggcctaccacgccatctcccgggccctggagaaggagggcctgaaggacaag |
| | | aagtcccccctgaacctgtcctccgagctgcaggacgagatcggcaccgccttctccctgttcaagaccgacgaggacatcaccggccggctgaagg |
| | | accgggtgcagcccgagatcctggaggccctgctgaagcacatctccttcgacaagttcgtgcagatctccctgaaggccctgcggcggatcgtgcc |
| | | cctgatggagcagggcaagcggtacgacgaggcctgcgccgagatctacggcgaccactacggcaagaagaacaccgaggagaagatctacctgccc |
| | | cccatccccgccgacgagatccggaaccccgtggtgctgcgggccctgtcccaggcccggaaggtgatcaacggcgtggtgcggcggtacggctccc |
| | | ccgcccggatccacatcgagaccgcccgggaggtgggcaagtccttcaaggaccggaaggagatcgagaagcggcaggaggagaaccggaaggaccg |
| | | ggagaaggccgccgccaagttccgggagtacttccccaacttcgtgggcgagcccaagtccaaggacatcctgaagctgcggctgtacgagcagcag |
| | | cacggcaagtgcctgtactccggcaaggagatcaacctggtgcggctgaacgagaagggctacgtggagatcgaccacgccctgcccttctcccgga |
| | | cctgggacgactccttcaacaacaaggtgctggtgctgggctccgagaaccagaacaagggcaaccagaccccctacgagtacttcaacggcaagga |
| | | caactcccgggagtggcaggagttcaaggcccgggtggagacctcccggttcccccggtccaagaagcagcggatcctgctgcagaagttcgacgag |
| | | gacggcttcaaggagtgcaacctgaacgacacccggtacgtgaaccggttcctgtgccagttcgtggccgaccacatcctgctgaccggcaagggca |
| | | agcggcgggtgttcgcctccaacggccagatcaccaacctgctgcggggcttctggggcctgcggaaggtgcgggccgagaacgaccggcaccacgc |
| | | cctggacgccgtggtggtggcctgctccaccgtggccatgcagcagaagatcacccggttcgtgcggtacaaggagatgaacgccttcgacggcaag |
| | | accatcgacaaggagaccggcaaggtgctgcaccagaagacccacttcccccagccctgggagttcttcgcccaggaggtgatgatccgggtgttcg |
| | | gcaagcccgacggcaagcccgagttcgaggaggccgacacccccgagaagctgcggaccctgctggccgagaagctgtcctcccggcccgaggccgt |
| | | gcacgagtacgtgacccccctgttcgtgtcccgggcccccaaccggaagatgtccggcgcccacaaggacaccctgcggtccgccaagcggttcgtg |
| | | aagcacaacgagaagatctccgtgaagcgggtgtggctgaccgagatcaagctggccgacctggagaacatggtgaactacaagaacggccgggaga |
| | | tcgagctgtacgaggccctgaaggcccggctggaggcctacggcggcaacgccaagcaggccttcgaccccaaggacaaccccttctacaagaaggg |
| | | cggccagctggtgaaggccgtgcgggtggagaagacccaggagtccggcgtgctgctgaacaagaagaacgcctacaccatcgccgacaacggcgac |
| | | atggtgcgggtggacgtgttctgcaaggtggacaagaagggcaagaaccagtacttcatcgtgcccatctacgcctggcaggtggccgagaacatcc |
| | | tgcccgacatcgactgcaagggctaccggatcgacgactcctacaccttctgcttctccctgcacaagtacgacctgatcgccttccagaaggacga |
| | | gaagtccaaggtggagttcgcctactacatcaactgcgactcctccaacggccggttctacctggcctggcacgacaagggctccaaggagcagcag |
| | | ttccggatctccacccagaacctggtgctgatccagaagtaccaggtgaacgagctgggcaaggagatccggccctgccggctgaagaagcggcccc |
| | | ccgtgcggTCCGGAAAGCGGACCGCCGACGGCTCCGGAGGAGGAAGCCCCGCCGCCAAGAAGAAGAAGCTGGACtag |
| Open | 378 | atgGACGGCTCCGGCGGCGGCTCCCCCAAGAAGAAGCGGAAGGTGGAGGACAAGCGGCCCGCCGCCACCAAGAAGGCCGGCCAGGCCAAGAAGAAGA |
| reading | | AGGGCGGCTCCGGCGGCGGCgccgccttcaagcccaaccccatcaactacatcctgggcctggacatcggcatcgcctccgtgggctgggccatggt |
| frame for | | ggagatcgacgaggaggagaaccccatccggctgatcgacctgggcgtgcgggtgttcgagcgggccgaggtgcccaagaccggcgactccctggcc |
| Nme2Cas9 | | atggcccggcggctggcccggtccgtgcggcggctgacccggcggcgggcccaccggctgctgcgggcccggcggctgctgaagcgggagggcgtgc |
| encoded by | | tgcaggccgccgacttcgacgagaacggcctgatcaagtccctgcccaacaccccctggcagctgcgggccgccgccctggaccggaagctgacccc |
| mRNA O | | cctggagtggtccgccgtgctgctgcacctgatcaagcaccggggctacctgtcccagcggaagaacgagggcgagaccgccgacaaggagctgggc |
| | | gccctgctgaagggcgtggccaacaacgcccacgccctgcagaccggcgacttccggacccccgccgagctggccctgaacaagttcgagaaggagt |
| | | ccggccacatccggaaccagcggggcgactactcccacaccttctcccggaaggacctgcaggccgagctgatcctgctgttcgagaagcagaagga |
| | | gttcggcaacccccacgtgtccggcggcctgaaggagggcatcgagaccctgctgatgacccagcggcccgccctgtccggcgacgccgtgcagaag |
| | | atgctgggccactgcaccttcgagcccgccgagcccaaggccgccaagaacacctacaccgccgagcggttcatctggctgaccaagctgaacaacc |
| | | tgcggatcctggagcagggctccgagcggcccctgaccgacaccgagcgggccaccctgatggacgagccctaccggaagtccaagctgacctacgc |
| | | ccaggcccggaagctgctgggcctggaggacaccgccttcttcaagggcctgcggtacggcaaggacaacgccgaggcctccaccctgatggagatg |
| | | aaggcctaccacgccatctcccgggccctggagaaggagggcctgaaggacaagaagtcccccctgaacctgtcctccgagctgcaggacgagatcg |
| | | gcaccgccttctccctgttcaagaccgacgaggacatcaccggccggctgaaggaccgggtgcagcccgagatcctggaggccctgctgaagcacat |
| | | ctccttcgacaagttcgtgcagatctccctgaaggccctgcggcggatcgtgcccctgatggagcagggcaagcggtacgacgaggcctgcgccgag |
| | | atctacggcgaccactacggcaagaagaacaccgaggagaagatctacctgccccccatccccgccgacgagatccggaaccccgtggtgctgcggg |
| | | ccctgtcccaggcccggaaggtgatcaacggcgtggtgcggcggtacggctcccccgcccggatccacatcgagaccgcccgggaggtgggcaagtc |
| | | cttcaaggaccggaaggagatcgagaagcggcaggaggagaaccggaaggaccgggagaaggccgccgccaagttccgggagtacttccccaacttc |
| | | gtgggcgagcccaagtccaaggacatcctgaagctgcggctgtacgagcagcagcacggcaagtgcctgtactccggcaaggagatcaacctggtgc |
| | | ggctgaacgagaagggctacgtggagatcgaccacgccctgcccttctcccggacctgggacgactccttcaacaacaaggtgctggtgctgggctc |
| | | cgagaaccagaacaagggcaaccagaccccctacgagtacttcaacggcaaggacaactcccgggagtggcaggagttcaaggcccgggtggagacc |
| | | tcccggttcccccggtccaagaagcagcggatcctgctgcagaagttcgacgaggacggcttcaaggagtgcaacctgaacgacacccggtacgtga |
| | | accggttcctgtgccagttcgtggccgaccacatcctgctgaccggcaagggcaagcggcgggtgttcgcctccaacggccagatcaccaacctgct |
| | | gcggggcttctggggcctgcggaaggtgcgggccgagaacgaccggcaccacgccctggacgccgtggtggtggcctgctccaccgtggccatgcag |
| | | cagaagatcacccggttcgtgcggtacaaggagatgaacgccttcgacggcaagaccatcgacaaggagaccggcaaggtgctgcaccagaagaccc |
| | | acttcccccagccctgggagttcttcgcccaggaggtgatgatccgggtgttcggcaagcccgacggcaagcccgagttcgaggaggccgacacccc |
| | | cgagaagctgcggaccctgctggccgagaagctgtcctcccggcccgaggccgtgcacgagtacgtgacccccctgttcgtgtcccgggcccccaac |
| | | cggaagatgtccggcgcccacaaggacaccctgcggtccgccaagcggttcgtgaagcacaacgagaagatctccgtgaagcgggtgtggctgaccg |
| | | agatcaagctggccgacctggagaacatggtgaactacaagaacggccgggagatcgagctgtacgaggccctgaaggcccggctggaggcctacgg |
| | | cggcaacgccaagcaggccttcgaccccaaggacaaccccttctacaagaagggcggccagctggtgaaggccgtgcgggtggagaagacccaggag |
| | | tccggcgtgctgctgaacaagaagaacgcctacaccatcgccgacaacggcgacatggtgcgggtggacgtgttctgcaaggtggacaagaagggca |
| | | agaaccagtacttcatcgtgcccatctacgcctggcaggtggccgagaacatcctgcccgacatcgactgcaagggctaccggatcgacgactccta |
| | | caccttctgcttctccctgcacaagtacgacctgatcgccttccagaaggacgagaagtccaaggtggagttcgcctactacatcaactgcgactcc |
| | | tccaacggccggttctacctggcctggcacgacaagggctccaaggagcagcagttccggatctccacccagaacctggtgctgatccagaagtacc |
| | | aggtgaacgagctgggcaaggagatccggccctgccggctgaagaagcggccccccgtgcggtag |
| Open | 379 | atgGACGGCTCCGGCGGCGGCTCCCCCAAGAAGAAGCGGAAGGTGGAGGACAAGCGGCCCGCCGCCACCAAGAAGGCCGGCCAGGCCAAGAAGAAGA |
| reading | | AGGGCGGCTCCGGCGGCGGCGCCGCCTTCAAGCCCAACCCCATCAACTACATCCTGGGCCTGGACATCGGCATCGCCTCCGTGGGCTGGGCCATGGT |
| frame for | | GGAGATCGACGAGGAGGAGAACCCCATCCGGCTGATCGACCTGGGCGTGCGGGTGTTCGAGCGGGCCGAGGTGCCCAAGACCGGCGACTCCCTGGCC |
| Nme2Cas9 | | ATGGCCCGGCGGCTGGCCCGGTCCGTGCGGCGGCTGACCCGGCGGCGGGCCCACCGGCTGCTGCGGGCCCGGCGGCTGCTGAAGCGGGAGGGCGTGC |
| encoded by | | TGCAGGCCGCCGACTTCGACGAGAACGGCCTGATCAAGTCCCTGCCCAACACCCCCTGGCAGCTGCGGGCCGCCGCCCTGGACCGGAAGCTGACCCC |
| mRNA P | | CCTGGAGTGGTCCGCCGTGCTGCTGCACCTGATCAAGCACCGGGGCTACCTGTCCCAGCGGAAGAACGAGGGCGAGACCGCCGACAAGGAGCTGGGC |
| | | GCCCTGCTGAAGGGCGTGGCCAACAACGCCCACGCCCTGCAGACCGGCGACTTCCGGACCCCCGCCGAGCTGGCCCTGAACAAGTTCGAGAAGGAGT |
| | | CCGGCCACATCCGGAACCAGCGGGGCGACTACTCCCACACCTTCTCCCGGAAGGACCTGCAGGCCGAGCTGATCCTGCTGTTCGAGAAGCAGAAGGA |
| | | GTTCGGCAACCCCCACGTGTCCGGCGGCCTGAAGGAGGGCATCGAGACCCTGCTGATGACCCAGCGGCCCGCCCTGTCCGGCGACGCCGTGCAGAAG |
| | | ATGCTGGGCCACTGCACCTTCGAGCCCGCCGAGCCCAAGGCCGCCAAGAACACCTACACCGCCGAGCGGTTCATCTGGCTGACCAAGCTGAACAACC |
| | | TGCGGATCCTGGAGCAGGGCTCCGAGCGGCCCCTGACCGACACCGAGCGGGCCACCCTGATGGACGAGCCCTACCGGAAGTCCAAGCTGACCTACGC |
| | | CCAGGCCCGGAAGCTGCTGGGCCTGGAGGACACCGCCTTCTTCAAGGGCCTGCGGTACGGCAAGGACAACGCCGAGGCCTCCACCCTGATGGAGATG |
| | | AAGGCCTACCACGCCATCTCCCGGGCCCTGGAGAAGGAGGGCCTGAAGGACAAGAAGTCCCCCCTGAACCTGTCCTCCGAGCTGCAGGACGAGATCG |
| | | GCACCGCCTTCTCCCTGTTCAAGACCGACGAGGACATCACCGGCCGGCTGAAGGACCGGGTGCAGCCCGAGATCCTGGAGGCCCTGCTGAAGCACAT |
| | | CTCCTTCGACAAGTTCGTGCAGATCTCCCTGAAGGCCCTGCGGCGGATCGTGCCCCTGATGGAGCAGGGCAAGCGGTACGACGAGGCCTGCGCCGAG |
| | | ATCTACGGCGACCACTACGGCAAGAAGAACACCGAGGAGAAGATCTACCTGCCCCCCATCCCCGCCGACGAGATCCGGAACCCCGTGGTGCTGCGGG |
| | | CCCTGTCCCAGGCCCGGAAGGTGATCAACGGCGTGGTGCGGCGGTACGGCTCCCCCGCCCGGATCCACATCGAGACCGCCCGGGAGGTGGGCAAGTC |
| | | CTTCAAGGACCGGAAGGAGATCGAGAAGCGGCAGGAGGAGAACCGGAAGGACCGGGAGAAGGCCGCCGCCAAGTTCCGGGAGTACTTCCCCAACTTC |
| | | GTGGGCGAGCCCAAGTCCAAGGACATCCTGAAGCTGCGGCTGTACGAGCAGCAGCACGGCAAGTGCCTGTACTCCGGCAAGGAGATCAACCTGGTGC |
| | | GGCTGAACGAGAAGGGCTACGTGGAGATCGACCACGCCCTGCCCTTCTCCCGGACCTGGGACGACTCCTTCAACAACAAGGTGCTGGTGCTGGGCTC |
| | | CGAGAACCAGAACAAGGGCAACCAGACCCCCTACGAGTACTTCAACGGCAAGGACAACTCCCGGGAGTGGCAGGAGTTCAAGGCCCGGGTGGAGACC |
| | | TCCCGGTTCCCCCGGTCCAAGAAGCAGCGGATCCTGCTGCAGAAGTTCGACGAGGACGGCTTCAAGGAGTGCAACCTGAACGACACCCGGTACGTGA |
| | | ACCGGTTCCTGTGCCAGTTCGTGGCCGACCACATCCTGCTGACCGGCAAGGGCAAGCGGCGGGTGTTCGCCTCCAACGGCCAGATCACCAACCTGCT |
| | | GCGGGGCTTCTGGGGCCTGCGGAAGGTGCGGGCCGAGAACGACCGGCACCACGCCCTGGACGCCGTGGTGGTGGCCTGCTCCACCGTGGCCATGCAG |
| | | CAGAAGATCACCCGGTTCGTGCGGTACAAGGAGATGAACGCCTTCGACGGCAAGACCATCGACAAGGAGACCGGCAAGGTGCTGCACCAGAAGACCC |
| | | ACTTCCCCCAGCCCTGGGAGTTCTTCGCCCAGGAGGTGATGATCCGGGTGTTCGGCAAGCCCGACGGCAAGCCCGAGTTCGAGGAGGCCGACACCCC |
| | | CGAGAAGCTGCGGACCCTGCTGGCCGAGAAGCTGTCCTCCCGGCCCGAGGCCGTGCACGAGTACGTGACCCCCCTGTTCGTGTCCCGGGCCCCCAAC |
| | | CGGAAGATGTCCGGCGCCCACAAGGACACCCTGCGGTCCGCCAAGCGGTTCGTGAAGCACAACGAGAAGATCTCCGTGAAGCGGGTGTGGCTGACCG |
| | | AGATCAAGCTGGCCGACCTGGAGAACATGGTGAACTACAAGAACGGCCGGGAGATCGAGCTGTACGAGGCCCTGAAGGCCCGGCTGGAGGCCTACGG |
| | | CGGCAACGCCAAGCAGGCCTTCGACCCCAAGGACAACCCCTTCTACAAGAAGGGCGGCCAGCTGGTGAAGGCCGTGCGGGTGGAGAAGACCCAGGAG |
| | | TCCGGCGTGCTGCTGAACAAGAAGAACGCCTACACCATCGCCGACAACGGCGACATGGTGCGGGTGGACGTGTTCTGCAAGGTGGACAAGAAGGGCA |
| | | AGAACCAGTACTTCATCGTGCCCATCTACGCCTGGCAGGTGGCCGAGAACATCCTGCCCGACATCGACTGCAAGGGCTACCGGATCGACGACTCCTA |
| | | CACCTTCTGCTTCTCCCTGCACAAGTACGACCTGATCGCCTTCCAGAAGGACGAGAAGTCCAAGGTGGAGTTCGCCTACTACATCAACTGCGACTCC |
| | | TCCAACGGCCGGTTCTACCTGGCCTGGCACGACAAGGGCTCCAAGGAGCAGCAGTTCCGGATCTCCACCCAGAACCTGGTGCTGATCCAGAAGTACC |
| | | AGGTGAACGAGCTGGGCAAGGAGATCCGGCCCTGCCGGCTGAAGAAGCGGCCCCCCGTGCGGTCCGAGTCCGCCACCCCCGAGTCCGTGTCCGGCTG |
| | | GCGGCTGTTCAAGAAGATCTCCTAG |
| Open | 380 | atgGACGGCTCCGGCGGCGGCTCCGAGGACAAGCGGCCCGCCGCCACCAAGAAGGCCGGCCAGGCCAAGAAGAAGAAGGGCGGCTCCGGCGGCGGCg |
| reading | | ccgccttcaagcccaaccccatcaactacatcctgggcctggacatcggcatcgcctccgtgggctgggccatggtggagatcgacgaggaggagaa |
| frame for | | ccccatccggctgatcgacctgggcgtgcgggtgttcgagcgggccgaggtgcccaagaccggcgactccctggccatggcccggcggctggcccgg |
| Nme2Cas9 | | tccgtgcggcggctgacccggcggcgggcccaccggctgctgcgggcccggcggctgctgaagcgggagggcgtgctgcaggccgccgacttcgacg |
| encoded by | | agaacggcctgatcaagtccctgcccaacaccccctggcagctgcgggccgccgccctggaccggaagctgacccccctggagtggtccgccgtgct |
| mRNA Q | | gctgcacctgatcaagcaccggggctacctgtcccagcggaagaacgagggcgagaccgccgacaaggagctgggcgccctgctgaagggcgtggcc |
| | | aacaacgcccacgccctgcagaccggcgacttccggacccccgccgagctggccctgaacaagttcgagaaggagtccggccacatccggaaccagc |
| | | ggggcgactactcccacaccttctcccggaaggacctgcaggccgagctgatcctgctgttcgagaagcagaaggagttcggcaacccccacgtgtc |
| | | cggcggcctgaaggagggcatcgagaccctgctgatgacccagcggcccgccctgtccggcgacgccgtgcagaagatgctgggccactgcaccttc |
| | | gagcccgccgagcccaaggccgccaagaacacctacaccgccgagcggttcatctggctgaccaagctgaacaacctgcggatcctggagcagggct |
| | | ccgagcggcccctgaccgacaccgagcgggccaccctgatggacgagccctaccggaagtccaagctgacctacgcccaggcccggaagctgctggg |
| | | cctggaggacaccgccttcttcaagggcctgcggtacggcaaggacaacgccgaggcctccaccctgatggagatgaaggcctaccacgccatctcc |
| | | cgggccctggagaaggagggcctgaaggacaagaagtcccccctgaacctgtcctccgagctgcaggacgagatcggcaccgccttctccctgttca |
| | | agaccgacgaggacatcaccggccggctgaaggaccgggtgcagcccgagatcctggaggccctgctgaagcacatctccttcgacaagttcgtgca |
| | | gatctccctgaaggccctgcggcggatcgtgcccctgatggagcagggcaagcggtacgacgaggcctgcgccgagatctacggcgaccactacggc |
| | | aagaagaacaccgaggagaagatctacctgccccccatccccgccgacgagatccggaaccccgtggtgctgcgggccctgtcccaggcccggaagg |
| | | tgatcaacggcgtggtgcggcggtacggctcccccgcccggatccacatcgagaccgcccgggaggtgggcaagtccttcaaggaccggaaggagat |
| | | cgagaagcggcaggaggagaaccggaaggaccgggagaaggccgccgccaagttccgggagtacttccccaacttcgtgggcgagcccaagtccaag |
| | | gacatcctgaagctgcggctgtacgagcagcagcacggcaagtgcctgtactccggcaaggagatcaacctggtgcggctgaacgagaagggctacg |
| | | tggagatcgaccacgccctgcccttctcccggacctgggacgactccttcaacaacaaggtgctggtgctgggctccgagaaccagaacaagggcaa |
| | | ccagaccccctacgagtacttcaacggcaaggacaactcccgggagtggcaggagttcaaggcccgggtggagacctcccggttcccccggtccaag |
| | | aagcagcggatcctgctgcagaagttcgacgaggacggcttcaaggagtgcaacctgaacgacacccggtacgtgaaccggttcctgtgccagttcg |
| | | tggccgaccacatcctgctgaccggcaagggcaagcggcgggtgttcgcctccaacggccagatcaccaacctgctgcggggcttctggggcctgcg |
| | | gaaggtgcgggccgagaacgaccggcaccacgccctggacgccgtggtggtggcctgctccaccgtggccatgcagcagaagatcacccggttcgtg |
| | | cggtacaaggagatgaacgccttcgacggcaagaccatcgacaaggagaccggcaaggtgctgcaccagaagacccacttcccccagccctgggagt |
| | | tcttcgcccaggaggtgatgatccgggtgttcggcaagcccgacggcaagcccgagttcgaggaggccgacacccccgagaagctgcggaccctgct |
| | | ggccgagaagctgtcctcccggcccgaggccgtgcacgagtacgtgacccccctgttcgtgtcccgggcccccaaccggaagatgtccggcgcccac |
| | | aaggacaccctgcggtccgccaagcggttcgtgaagcacaacgagaagatctccgtgaagcgggtgtggctgaccgagatcaagctggccgacctgg |
| | | agaacatggtgaactacaagaacggccgggagatcgagctgtacgaggccctgaaggcccggctggaggcctacggcggcaacgccaagcaggcctt |
| | | cgaccccaaggacaaccccttctacaagaagggcggccagctggtgaaggccgtgcgggtggagaagacccaggagtccggcgtgctgctgaacaag |
| | | aagaacgcctacaccatcgccgacaacggcgacatggtgcgggtggacgtgttctgcaaggtggacaagaagggcaagaaccagtacttcatcgtgc |
| | | ccatctacgcctggcaggtggccgagaacatcctgcccgacatcgactgcaagggctaccggatcgacgactcctacaccttctgcttctccctgca |
| | | caagtacgacctgatcgccttccagaaggacgagaagtccaaggtggagttcgcctactacatcaactgcgactcctccaacggccggttctacctg |
| | | gcctggcacgacaagggctccaaggagcagcagttccggatctccacccagaacctggtgctgatccagaagtaccaggtgaacgagctgggcaagg |
| | | agatccggccctgccggctgaagaagcggccccccgtgcggtag |
| Open | 381 | ATGGACGGCTCCGGCGGCGGCTCCCCCAAGAAGAAGCGGAAGGTGGAGGACAAGCGGCCCGCCGCCACCAAGAAGGCCGGCCAGGCCAAGAAGAAGA |
| reading | | AGGGCGGCTCCGGCGGCGGCGAGGCCTCCCCCGCCTCCGGCCCCCGGCACCTGATGGACCCCCACATCTTCACCTCCAACTTCAACAACGGCATCGG |
| frame for | | CCGGCACAAGACCTACCTGTGCTACGAGGTGGAGCGGCTGGACAACGGCACCTCCGTGAAGATGGACCAGCACCGGGGCTTCCTGCACAACCAGGCC |
| Nme2Cas9 | | AAGAACCTGCTGTGCGGCTTCTACGGCCGGCACGCCGAGCTGCGGTTCCTGGACCTGGTGCCCTCCCTGCAGCTGGACCCCGCCCAGATCTACCGGG |
| base editor | | TGACCTGGTTCATCTCCTGGTCCCCCTGCTTCTCCTGGGGCTGCGCCGGCGAGGTGCGGGCCTTCCTGCAGGAGAACACCCACGTGCGGCTGCGGAT |
| encoded by | | CTTCGCCGCCCGGATCTACGACTACGACCCCCTGTACAAGGAGGCCCTGCAGATGCTGCGGGACGCCGGCGCCCAGGTGTCCATCATGACCTACGAC |
| mRNA S | | GAGTTCAAGCACTGCTGGGACACCTTCGTGGACCACCAGGGCTGCCCCTTCCAGCCCTGGGACGGCCTGGACGAGCACTCCCAGGCCCTGTCCGGCC |
| | | GGCTGCGGGCCATCCTGCAGAACCAGGGCAACTCCGGCTCCGAGACCCCCGGCACCTCCGAGTCCGCCACCCCCGAGTCCGCAGCGTTCAAACCAAA |
| | | Tcccatcaactacatcctgggcctggccatcggcatcgcctccgtgggctgggccatggtggagatcgacgaggaggagaaccccatccggctgatc |
| | | gacctgggcgtgcgggtgttcgagcgggccgaggtgcccaagaccggcgactccctggccatggcccggcggctggcccggtccgtgcggcggctga |
| | | cccggcggcgggcccaccggctgctgcgggcccggcggctgctgaagcgggagggcgtgctgcaggccgccgacttcgacgagaacggcctgatcaa |
| | | gtccctgcccaacaccccctggcagctgcgggccgccgccctggaccggaagctgacccccctggagtggtccgccgtgctgctgcacctgatcaag |
| | | caccggggctacctgtcccagcggaagaacgagggcgagaccgccgacaaggagctgggcgccctgctgaagggcgtggccaacaacgcccacgccc |
| | | tgcagaccggcgacttccggacccccgccgagctggccctgaacaagttcgagaaggagtccggccacatccggaaccagcggggcgactactccca |
| | | caccttctcccggaaggacctgcaggccgagctgatcctgctgttcgagaagcagaaggagttcggcaacccccacgtgtccggcggcctgaaggag |
| | | ggcatcgagaccctgctgatgacccagcggcccgccctgtccggcgacgccgtgcagaagatgctgggccactgcaccttcgagcccgccgagccca |
| | | aggccgccaagaacacctacaccgccgagcggttcatctggctgaccaagctgaacaacctgcggatcctggagcagggctccgagcggcccctgac |
| | | cgacaccgagcgggccaccctgatggacgagccctaccggaagtccaagctgacctacgcccaggcccggaagctgctgggcctggaggacaccgcc |
| | | ttcttcaagggcctgcggtacggcaaggacaacgccgaggcctccaccctgatggagatgaaggcctaccacgccatctcccgggccctggagaagg |
| | | agggcctgaaggacaagaagtcccccctgaacctgtcctccgagctgcaggacgagatcggcaccgccttctccctgttcaagaccgacgaggacat |
| | | caccggccggctgaaggaccgggtgcagcccgagatcctggaggccctgctgaagcacatctccttcgacaagttcgtgcagatctccctgaaggcc |
| | | ctgcggcggatcgtgcccctgatggagcagggcaagcggtacgacgaggcctgcgccgagatctacggcgaccactacggcaagaagaacaccgagg |
| | | agaagatctacctgccccccatccccgccgacgagatccggaaccccgtggtgctgcgggccctgtcccaggcccggaaggtgatcaacggcgtggt |
| | | gcggcggtacggctcccccgcccggatccacatcgagaccgcccgggaggtgggcaagtccttcaaggaccggaaggagatcgagaagcggcaggag |
| | | gagaaccggaaggaccgggagaaggccgccgccaagttccgggagtacttccccaacttcgtgggcgagcccaagtccaaggacatcctgaagctgc |
| | | ggctgtacgagcagcagcacggcaagtgcctgtactccggcaaggagatcaacctggtgcggctgaacgagaagggctacgtggagatcgaccacgc |
| | | cctgcccttctcccggacctgggacgactccttcaacaacaaggtgctggtgctgggctccgagaaccagaacaagggcaaccagaccccctacgag |
| | | tacttcaacggcaaggacaactcccgggagtggcaggagttcaaggcccgggtggagacctcccggttcccccggtccaagaagcagcggatcctgc |
| | | tgcagaagttcgacgaggacggcttcaaggagtgcaacctgaacgacacccggtacgtgaaccgcttcctgtgccagttcgtggccgaccacatcct |
| | | gctgaccggcaagggcaagcggcgggtgttcgcctccaacggccagatcaccaacctgctgcggggcttctggggcctgcggaaggtgcgggccgag |
| | | aacgaccggcaccacgccctggacgccgtggtggtggcctgctccaccgtggccatgcagcagaagatcacccggttcgtgcggtacaaggagatga |
| | | acgccttcgacggcaagaccatcgacaaggagaccggcaaggtgctgcaccagaagacccacttcccccagccctgggagttcttcgcccaggaggt |
| | | gatgatccgggtgttcggcaagcccgacggcaagcccgagttcgaggaggccgacacccccgagaagctgcggaccctgctggccgagaagctgtcc |
| | | tcccggcccgaggccgtgcacgagtacgtgacccccctgttcgtgtcccgggcccccaaccggaagatgtccggcgcccacaaggacaccctgcggt |
| | | ccgccaagcggttcgtgaagcacaacgagaagatctccgtgaagcgggtgtggctgaccgagatcaagctggccgacctggagaacatggtgaacta |
| | | caagaacggccgggagatcgagctgtacgaggccctgaaggcccggctggaggcctacggcggcaacgccaagcaggccttcgaccccaaggacaac |
| | | cccttctacaagaagggcggccagctggtgaaggccgtgcgggtggagaagacccaggagtccggcgtgctgctgaacaagaagaacgcctacacca |
| | | tcgccgacaacggcgacatggtgcgggtggacgtgttctgcaaggtggacaagaagggcaagaaccagtacttcatcgtgcccatctacgcctggca |
| | | ggtggccgagaacatcctgcccgacatcgactgcaagggctaccggatcgacgactcctacaccttctgcttctccctgcacaagtacgacctgatc |
| | | gccttccagaaggacgagaagtccaaggtggagttcgcctactacatcaactgcgactcctccaacggccggttctacctggcctggcacgacaagg |
| | | gctccaaggagcagcagttccggatctccacccagaacctggtgctgatccagaagtaccaggtgaacgagctgggcaaggagatccggccctgccg |
| | | gctgaagaagcggccccccgtgcggtag |
| open | 382 | ATGgaggcctcccccgcctccggcccccggcacctgatggacccccacatcttcacctccAACTTCAACAACggcATCggccggCACAAGaccTACC |
| reading | | TGTGCTACgaggtggagcggCTGGACAACggcacctccgtgAAGATGGACCAGCACcggggcTTCCTGCACAACCAGgccAAGAACCTGCTGTGCgg |
| frame for | | cTTCTACggccggCACgccgagCTGcggTTCCTGGACCTGgtgccctccCTGCAGCTGGACcccgccCAGATCTACcgggtgaccTGGTTCATCtcc |
| BC22n | | TGGtcccccTGCTTCtccTGGggcTGCgccggcgaggtgcgggccTTCCTGCAGgagAACaccCACgtgcggCTGcggATCTTCgccgcccggATCT |
| SpyCas9 | | ACGACTACGACcccCTGTACAAGgaggccCTGCAGATGCTGcggGACgccggcgccCAGgtgtccATCATGaccTACGACgagTTCAAGCACTGCTG |
| base editor | | GGACaccTTCgtgGACCACCAGggcTGCcccTTCCAGcccTGGGACggcCTGGACgagCACtccCAGgccCTGtccggccggCTGcgggccATCCTG |
| encoded by | | CAGAACCAGggcAACtccggctccgagacccccggcacctccgagtccgccacccccgagtccgacaagaagtactccatcggcctggCcatcggca |
| mRNA E | | ccaactccgtgggctgggccgtgatcaccgacgagtacaaggtgccctccaagaagttcaaggtgctgggcaacaccgaccggcactccatcaagaa |
| | | gaacctgatcggcgccctgctgttcgactccggcgagaccgccgaggccacccggctgaagcggaccgcccggcggcggtacacccggcggaagaac |
| | | cggatctgctacctgcaggagatcttctccaacgagatggccaaggtggacgactccttcttccaccggctggaggagtccttcctggtggaggagg |
| | | acaagaagcacgagcggcaccccatcttcggcaacatcgtggacgaggtggcctaccacgagaagtaccccaccatctaccacctgcggaagaagct |
| | | ggtggactccaccgacaaggccgacctgcggctgatctacctggccctggcccacatgatcaagttccggggccacttcctgatcgagggcgacctg |
| | | aaccccgacaactccgacgtggacaagctgttcatccagctggtgcagacctacaaccagctgttcgaggagaaccccatcaacgcctccggcgtgg |
| | | acgccaaggccatcctgtccgcccggctgtccaagtcccggcggctggagaacctgatcgcccagctgcccggcgagaagaagaacggcctgttcgg |
| | | caacctgatcgccctgtccctgggcctgacccccaacttcaagtccaacttcgacctggccgaggacgccaagctgcagctgtccaaggacacctac |
| | | gacgacgacctggacaacctgctggcccagatcggcgaccagtacgccgacctgttcctggccgccaagaacctgtccgacgccatcctgctgtccg |
| | | acatcctgcgggtgaacaccgagatcaccaaggcccccctgtccgcctccatgatcaagcggtacgacgagcaccaccaggacctgaccctgctgaa |
| | | ggccctggtgcggcagcagctgcccgagaagtacaaggagatcttcttcgaccagtccaagaacggctacgccggctacatcgacggcggcgcctcc |
| | | caggaggagttctacaagttcatcaagcccatcctggagaagatggacggcaccgaggagctgctggtgaagctgaaccgggaggacctgctgcgga |
| | | agcagcggaccttcgacaacggctccatcccccaccagatccacctgggcgagctgcacgccatcctgcggcggcaggaggacttctaccccttcct |
| | | gaaggacaaccgggagaagatcgagaagatcctgaccttccggatcccctactacgtgggccccctggcccggggcaactcccggttcgcctggatg |
| | | acccggaagtccgaggagaccatcaccccctggaacttcgaggaggtggtggacaagggcgcctccgcccagtccttcatcgagcggatgaccaact |
| | | tcgacaagaacctgcccaacgagaaggtgctgcccaagcactccctgctgtacgagtacttcaccgtgtacaacgagctgaccaaggtgaagtacgt |
| | | gaccgagggcatgcggaagcccgccttcctgtccggcgagcagaagaaggccatcgtggacctgctgttcaagaccaaccggaaggtgaccgtgaag |
| | | cagctgaaggaggactacttcaagaagatcgagtgcttcgactccgtggagatctccggcgtggaggaccggttcaacgcctccctgggcacctacc |
| | | acgacctgctgaagatcatcaaggacaaggacttcctggacaacgaggagaacgaggacatcctggaggacatcgtgctgaccctgaccctgttcga |
| | | ggaccgggagatgatcgaggagcggctgaagacctacgcccacctgttcgacgacaaggtgatgaagcagctgaagcggcggcggtacaccggctgg |
| | | ggccggctgtcccggaagctgatcaacggcatccgggacaagcagtccggcaagaccatcctggacttcctgaagtccgacggcttcgccaaccgga |
| | | acttcatgcagctgatccacgacgactccctgaccttcaaggaggacatccagaaggcccaggtgtccggccagggcgactccctgcacgagcacat |
| | | cgccaacctggccggctcccccgccatcaagaagggcatcctgcagaccgtgaaggtggtggacgagctggtgaaggtgatgggccggcacaagccc |
| | | gagaacatcgtgatcgagatggcccgggagaaccagaccacccagaagggccagaagaactcccgggagcggatgaagcggatcgaggagggcatca |
| | | aggagctgggctcccagatcctgaaggagcaccccgtggagaacacccagctgcagaacgagaagctgtacctgtactacctgcagaacggccggga |
| | | catgtacgtggaccaggagctggacatcaaccggctgtccgactacgacgtggaccacatcgtgccccagtccttcctgaaggacgactccatcgac |
| | | aacaaggtgctgacccggtccgacaagaaccggggcaagtccgacaacgtgccctccgaggaggtggtgaagaagatgaagaactactggcggcagc |
| | | tgctgaacgccaagctgatcacccagcggaagttcgacaacctgaccaaggccgagcggggcggcctgtccgagctggacaaggccggcttcatcaa |
| | | gcggcagctggtggagacccggcagatcaccaagcacgtggcccagatcctggactcccggatgaacaccaagtacgacgagaacgacaagctgatc |
| | | cgggaggtgaaggtgatcaccctgaagtccaagctggtgtccgacttccggaaggacttccagttctacaaggtgcgggagatcaacaactaccacc |
| | | acgcccacgacgcctacctgaacgccgtggtgggcaccgccctgatcaagaagtaccccaagctggagtccgagttcgtgtacggcgactacaaggt |
| | | gtacgacgtgcggaagatgatcgccaagtccgagcaggagatcggcaaggccaccgccaagtacttcttctactccaacatcatgaacttcttcaag |
| | | accgagatcaccctggccaacggcgagatccggaagcggcccctgatcgagaccaacggcgagaccggcgagatcgtgtgggacaagggccgggact |
| | | tcgccaccgtgcggaaggtgctgtccatgccccaggtgaacatcgtgaagaagaccgaggtgcagaccggcggcttctccaaggagtccatcctgcc |
| | | caagcggaactccgacaagctgatcgcccggaagaaggactgggaccccaagaagtacggcggcttcgactcccccaccgtggcctactccgtgctg |
| | | gtggtggccaaggtggagaagggcaagtccaagaagctgaagtccgtgaaggagctgctgggcatcaccatcatggagcggtcctccttcgagaaga |
| | | accccatcgacttcctggaggccaagggctacaaggaggtgaagaaggacctgatcatcaagctgcccaagtactccctgttcgagctggagaacgg |
| | | ccggaagcggatgctggcctccgccggcgagctgcagaagggcaacgagctggccctgccctccaagtacgtgaacttcctgtacctggcctcccac |
| | | tacgagaagctgaagggctcccccgaggacaacgagcagaagcagctgttcgtggagcagcacaagcactacctggacgagatcatcgagcagatct |
| | | ccgagttctccaagcgggtgatcctggccgacgccaacctggacaaggtgctgtccgcctacaacaagcaccgggacaagcccatccgggagcaggc |
| | | cgagaacatcatccacctgttcaccctgaccaacctgggcgcccccgccgccttcaagtacttcgacaccaccatcgaccggaagcggtacacctcc |
| | | accaaggaggtgctggacgccaccctgatccaccagtccatcaccggcctgtacgagacccggatcgacctgtcccagctgggcggcgacggcggcg |
| | | gctcccccaagaagaagcggaaggtgTgA |

### VI. EXAMPLES

The following examples are provided to illustrate certain disclosed embodiments and are not to be construed as limiting the scope of this disclosure in any way.

### Example 1. Materials and Methods

In Vitro Transcription (“IVT”) of Nuclease mRNA

Capped and polyadenylated mRNA containing N1-methyl pseudo-U was generated by in vitro transcription using routine methods. Typically, a plasmid DNA containing a T7 promoter, a sequence for transcription, and a polyadenylation region was linearized with XbaI per manufacturer's protocol. The XbaI was inactivated by heating. The linearized plasmid was purified from enzyme and buffer salts. The IVT reaction to generate modified mRNA was performed by incubating at 37° C.: 50 ng/μL linearized plasmid; 2-5 mM each of GTP, ATP, CTP, and N1-methyl pseudo-UTP (Trilink); 10-25 mM ARCA (Trilink); 5 U/μL T7 RNA polymerase; 1 U/μL Murine RNase inhibitor (NEB); 0.004 U/μL Inorganic E. coli pyrophosphatase (NEB); and 1× reaction buffer. TURBO DNase (ThermoFisher) was added to a final concentration of 0.01 U/μL, and the reaction was incubated at 37° C. to remove the DNA template.

The mRNA was purified using a MegaClear Transcription Clean-up kit (ThermoFisher) or a RNeasy Maxi kit (Qiagen) per the manufacturers' protocols. Alternatively, the mRNA was purified through a precipitation protocol, which in some cases was followed by HPLC-based purification. Briefly, after the DNase digestion, mRNA was purified using LiCl precipitation, ammonium acetate precipitation, and sodium acetate precipitation. For HPLC purified mRNA, after the LiCl precipitation and reconstitution, the mRNA was purified by RP-IP HPLC (see, e.g., Kariko, et al. Nucleic Acids Research, 2011, Vol. 39, No. 21 e142). The fractions chosen for pooling were combined and desalted by sodium acetate/ethanol precipitation as described above. In a further alternative method, mRNA was purified with a LiCl precipitation method followed by further purification by tangential flow filtration. RNA concentrations were determined by measuring the light absorbance at 260 nm (Nanodrop), and transcripts were analyzed by capillary electrophoresis by Bioanlayzer (Agilent).

Streptococcus pyogenes (“Spy”) Cas9 mRNA was generated from plasmid DNA encoding an open reading frame according to SEQ ID Nos: 321-323 (see sequences in Table 5). When the sequences cited in this paragraph are referred to below with respect to RNAs, it is understood that Ts should be replaced with Us (which can be modified nucleosides as described above). Messenger RNAs used in the Examples include a 5′ cap and a 3′ polyadenylation sequence e.g., up to 100 nucleotides. Guide RNAs were chemically synthesized by commercial vendors or using standard in vitro synthesis techniques with modified nucleotides.

Cell Preparation

Short sgRNAs targeting the mouse, rat, human, and cynomolgus (cyno) transthyretin TTR gene were designed and used for lipofection as described below, into primary mouse hepatocytes (PMH), primary rat hepatocytes (PRH), primary human hepatocytes (PHH), and primary cynomolgus hepatocytes (PCH), respectively. PMH, PRH, PHH, or PCH were thawed and resuspended in hepatocyte thawing medium with plating supplements (William's E Medium (Gibco, Cat. A12176-01, Lot 2039733)) with dexamethasone+cocktail supplement (Gibco, Cat. A15563, Lot 2019842) and Plating Supplements with FBS content (Gibco, Cat. A13450, Lot 1970698) followed by centrifugation. The supernatant was discarded and the pelleted cells resuspended in hepatocyte plating medium plus supplement pack (Invitrogen, Cat. A1217601 and Gibco, Cat. CM3000). Cells were counted and plated on Bio-coat collagen I coated 96-well plates (ThermoFisher, Cat. 877272). Plated cells were allowed to settle and adhere for 4-6 hours in a tissue culture incubator at 37° C. and 5% CO2 atmosphere. After incubation, cells were checked for monolayer formation and were washed once with hepatocyte maintenance medium (Invitrogen, Cat. A1217601 and Gibco, Cat. CM4000).

Preparation of LNP Formulation Containing sgRNA and Cas9 mRNA

In general, the lipid nanoparticle components were dissolved in 100% ethanol at various molar ratios. The RNA cargos (e.g., Cas9 mRNA and sgRNA) were dissolved in 25 mM citrate, 100 mM NaCl, pH 5.0, resulting in a concentration of RNA cargo of approximately 0.45 mg/mL. The LNPs used contained ionizable lipid ((9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate), also called herein Lipid A, cholesterol, distearoylphosphatidylcholine (DSPC), and 1,2-dimyristoyl-rac-glycero-3-methylpolyoxyethylene glycol 2000 (PEG2k-DMG) in a molar ratio of 50% Lipid A, 38% cholesterol, 9% DSPC, and 3% PEG2k-DMG. The LNPs were formulated with a lipid amine to RNA phosphate (N:P) molar ratio of about 6, and a ratio of gRNA to mRNA of 1:2 by weight. The LNPs used comprise a single RNA species such as Cas9 mRNA or a sgRNA. LNP are similarly prepared with a mixture of Cas9 mRNA and a guide RNA.

The LNPs were prepared using a cross-flow technique utilizing impinging jet mixing of the lipid in ethanol with two volumes of RNA solutions and one volume of water. The lipid in ethanol was mixed through a mixing cross with the two volumes of RNA solution. A fourth stream of water was mixed with the outlet stream of the cross through an inline tee (See WO2016010840 FIG. 2.). The LNPs were held for 1 hour at room temperature, and further diluted with water (approximately 1:1 v/v). Diluted LNPs were concentrated using tangential flow filtration on a flat sheet cartridge (Sartorius, 100 kD MWCO) and then buffer exchanged using PD-10 desalting columns (GE) into 50 mM Tris, 45 mM NaCl, 5% (w/v) sucrose, pH 7.5 (TSS). The resulting mixture was then filtered using a 0.2 μm sterile filter. The final LNPs were characterized to determine the encapsulation efficiency, polydispersity index, and average particle size. The final LNP was stored at 4° C. or −80° C. until further use.

sgRNA and Cas9 mRNA Lipofection

Lipofection of Cas9 mRNA and gRNAs used pre-mixed lipid formulations. The lipofection reagent contained ionizable lipid ((9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate), cholesterol, DSPC, and PEG2k-DMG in a molar ratio of 50% Lipid A, 38% cholesterol, 9% DSPC, and 3% PEG2k-DMG. This mixture was reconstituted in 100% ethanol then mixed with RNA cargos (e.g., Cas9 mRNA and gRNA) at a lipid amine to RNA phosphate (N:P) molar ratio of about 6.0. Guide RNA was chemically synthesized by commercial vendors or using standard in vitro synthesis techniques with modified nucleotides. An mRNA comprising a Cas9 ORF of Table 5 was produced by in vitro transcription (IVT) as described in WO2019/067910, see e.g. ¶354, using a 2 hour IVT reaction time and purifying the mRNA by LiCl precipitation followed by tangential flow filtration.

Lipofections were performed with a ratio of gRNA to mRNA of 1:1 by weight. Briefly, cells were incubated at 37° C., 5% CO2 for 24 hours prior to treatment with LNPs. LNPs were incubated in media containing 6% cynomolgus monkey or 6% fetal bovine serum (FBS) at 37° C. for 10 minutes. Post-incubation, the LNPs were added to the mouse or cynomolgus hepatocytes in an 8 or 12 point 3-fold dose response curve starting at 300 ng Cas9 mRNA. The cells were lysed 72 hours post-treatment for NGS analysis as described in Example 1.

Genomic DNA Isolation

Cells were harvested post-transfection at 72 hours. The gDNA was extracted from each well of a 96-well plate using 50 μL/well QuickExtract DNA Extraction solution (Epicentre, Cat. QE09050) or Quick Extract (Lucigen, Cat. SS000035-D2) according to manufacturer's protocol.

Next-Generation Sequencing (“NGS”) and Analysis for Editing Efficiency

To quantitatively determine the efficiency of editing at the target location in the genome, sequencing was utilized to identify the presence of insertions and deletions introduced by gene editing. PCR primers were designed around the target site within the gene of interest (e.g. TTR), and the genomic area of interest was amplified. Primer sequence design was done as is standard in the field.

Additional PCR was performed according to the manufacturer's protocols (Illumina) to add chemistry for sequencing. The amplicons were sequenced on an Illumina MiSeq instrument. The reads were aligned to the reference genome (e.g., hg38) after eliminating those having low quality scores. The resulting files containing the reads were mapped to the reference genome (BAM files), where reads that overlapped the target region of interest were selected and the number of wild type reads versus the number of reads which contain an insertion or deletion (“indel”) was calculated.

The editing percentage (e.g., the “editing efficiency” or “percent editing”) is defined as the total number of sequence reads with insertions or deletions (“indels”) over the total number of sequence reads, including wild type.

### Example 2. In Vitro Editing in Primary Hepatocytes

sgRNAs sharing the same targeting sequence, which is cross-reactive to mouse, cynomolgus monkey, and human TTR genes, with various scaffold sequences were designed as shown in Tables 2A-2B and lipofected into primary mouse (PMH), cynomolgus monkey (PCH), and human (PHH) hepatocytes. Cells from In Vitro ADMET Laboratories, Inc. and Gibco™ were prepared, treated by lipofection and analyzed as described above unless otherwise noted. Specifically, PMH (Lot #839), PCH (Lot #10136011), and PHH (Lot #8296) cells were used and plated at densities of 15,000, 30,000, and 33,000 cells/well, respectively. Lipofection reagent was prepared as described in Example 1 using a molar ratio of 50% Lipid A, 38% cholesterol, 9% DSPC, and 3% PEG2k-DMG. Lipofection samples were prepared using an N:P molar ratio of about 6 and a gRNA:mRNA ratio of 1:1 by weight. Duplicate samples were included in the assay. Mean editing results with standard deviation (SD) are shown in Table 6 and FIG. 1A for PMH, FIG. 1B for PCH, and FIG. 1C for PHH. NA indicates that one of the replicates for a guide did not satisfy the sequencing quality metrics so that no SD could be calculated.

TABLE 6
In vitro editing in PMH, PCH, PHH

| | PMH | | PCH | | PHH | |
|---|---|---|---|---|---|---|
| | Mean | | Mean | | Mean | |
| Guide ID | % Edit | SD | % Edit | SD | % Edit | SD |
| G018707 | 93.1 | 0.8 | 93.3 | 1.8 | 52.2 | 0.6 |
| G018675 | 91.5 | 3.5 | 82.0 | NA | 49.8 | 8.8 |
| G018681 | 90.7 | 3.1 | 91.1 | 1.3 | 52.4 | 12.0 |
| G018706 | 90.7 | 3.3 | 90.3 | NA | 61.5 | 0.1 |
| G018668 | 89.8 | 1.3 | 73.4 | 3.4 | 37.9 | 4.3 |
| G018684 | 89.7 | 4.4 | 85.2 | 5.1 | 65.6 | NA |
| G018695 | 89.6 | 2.8 | 84.1 | 3.7 | 28.1 | 1.6 |
| G018683 | 89.4 | 2.0 | 79.3 | 11.7 | 56.6 | 1.1 |
| G018674 | 88.9 | 7.1 | 85.2 | 10.2 | 42.1 | 5.5 |
| G018687 | 88.7 | 2.5 | 93.5 | 2.1 | 51.6 | 5.9 |
| G018667 | 88.3 | 3.2 | 79.1 | 1.3 | 40.1 | 8.8 |
| G018689 | 88.2 | 1.2 | 85.1 | 4.4 | 60.3 | 13.2 |
| G018685 | 88.0 | 4.1 | 78.0 | 5.7 | 46.8 | 3.7 |
| G018698 | 88.0 | 2.3 | 90.0 | 1.6 | 50.1 | 0.4 |
| G018694 | 87.8 | 7.5 | 91.3 | NA | 42.5 | 9.7 |
| G018705 | 87.8 | 1.4 | 88.1 | 4.0 | 57.7 | 1.8 |
| G018677 | 87.6 | 3.6 | 76.5 | 7.8 | 45.0 | 2.2 |
| G018690 | 87.4 | 3.0 | 91.9 | 0.5 | 60.3 | 7.6 |
| G000502 | 87.1 | 7.2 | 90.2 | 6.4 | 52.5 | 4.2 |
| G018702 | 86.4 | 1.1 | 85.0 | 1.1 | 32.9 | 0.8 |
| G018682 | 86.0 | 9.3 | 77.2 | NA | 37.8 | 16.6 |
| G018686 | 86.0 | 5.0 | 87.3 | 5.1 | 46.1 | 6.7 |
| G018700 | 85.8 | 12.2 | 84.3 | NA | 41.7 | 0.3 |
| G018676 | 84.8 | 0.1 | 87.0 | 2.8 | 42.4 | 8.1 |
| G018669 | 82.0 | 3.6 | 79.2 | 2.4 | 35.5 | 0.6 |
| G018693 | 81.7 | 8.8 | 82.5 | 5.4 | 42.0 | 6.6 |
| G018692 | 81.6 | 7.6 | 86.4 | 3.9 | 49.2 | 8.9 |
| G018688 | 81.3 | 4.8 | 87.9 | 2.1 | 57.0 | 4.4 |
| G018666 | 80.8 | 6.4 | 85.4 | 3.9 | 25.0 | 1.1 |
| G018701 | 80.1 | 2.7 | 77.5 | 12.3 | 26.1 | 2.8 |
| G018673 | 80.0 | 8.9 | 89.2 | 3.3 | 36.1 | 0.2 |
| G018678 | 78.8 | 2.5 | 81.0 | 2.2 | 28.8 | 7.4 |
| G018696 | 76.7 | 7.4 | 87.3 | 3.5 | 36.4 | 3.9 |
| G018699 | 76.1 | 0.4 | 82.1 | 8.6 | 34.3 | 6.4 |
| G018704 | 76.1 | 0.1 | 78.7 | 12.5 | 24.9 | 4.2 |
| G018691 | 75.8 | 3.3 | 90.6 | 5.2 | 43.0 | 5.0 |
| G018697 | 72.9 | 2.6 | 82.8 | 1.3 | 27.2 | 11.2 |
| G018703 | 69.5 | 0.0 | 70.6 | 4.1 | 13.8 | 4.2 |
| G018671 | 67.5 | 0.6 | 68.2 | 11.0 | 15.1 | 1.7 |
| G018670 | 62.3 | 4.1 | 47.7 | 9.3 | 9.6 | 0.2 |
| G018679 | 58.0 | 13.8 | 42.3 | 2.5 | 10.6 | 3.7 |
| G018672 | 55.0 | 5.7 | 56.2 | 2.5 | 7.8 | 0.6 |
| G018708 | 50.7 | 11.6 | 71.5 | 8.1 | 14.2 | 1.3 |
| G018680 | 12.6 | 0.6 | 12.3 | NA | 1.2 | 0.4 |
| G017278 | 92.7 | 5.8 | 87.9 | 5.3 | 60.3 | 6.6 |
| G012401 | 89.7 | 4.1 | 90.6 | 1.4 | 54.1 | 4.7 |

### Example 3. In Vitro Editing in Primary Hepatocytes with Dilution Curve

sgRNAs all having the same targeting sequence which is cross-reactive with mouse, human, cynomolgus monkey TTR genes, were designed with various scaffold sequences as shown in Tables 2A-2B that incorporated PEG linkers into different regions of the sgRNA constant region. Guides and Cas9 mRNA were lipofected into primary mouse hepatoctyes (PMH) as described above. PMH (Lot #839) cells were used and plated at a density of 15,000 cells/well. Cells from Gibco™ were prepared, treated by lipofection and analyzed as described above unless otherwise noted. Guides were assayed in an 8 point 3-fold dilution curve starting at 46.5 nM guide concentration as shown in Table 7. Two sets of guides were tested with control guides G000502 and G012401. Samples were run in triplicate. EC50 values and mean editing results are shown in Table 7. Dose response curves are plotted in FIG. 2A and FIG. 2B.

TABLE 7
Percent editing in PMH

| | Set 1 | | | | Set 2 | | | | |
|---|---|---|---|---|---|---|---|---|---|
| | Guide | Mean | | | Guide | Guide | Mean | | |
| Guide ID | conc. (nM) | % Edit | SD | EC50 | ID | conc. (nM) | % Edit | SD | EC50 |
| G000502 | 46.56 | 93.3 | 2.3 | 2.32 | G000502 | 46.56 | 92.9 | 1.7 | 3.25 |
| | 15.52 | 95.4 | 1.3 | | | 15.52 | 88.1 | 6.1 | |
| | 5.17 | 81.0 | 2.5 | | | 5.17 | 65.2 | 0.1 | |
| | 1.72 | 32.9 | 2.1 | | | 1.72 | 23.1 | 0.4 | |
| | 0.57 | 6.0 | 0.1 | | | 0.57 | 4.0 | 0.7 | |
| | 0.19 | 1.1 | 0.1 | | | 0.19 | 0.8 | 0.8 | |
| | 0.06 | 0.2 | 0.1 | | | 0.06 | 0.2 | 0.0 | |
| | 0.02 | 0.1 | 0.0 | | | 0.02 | 0.1 | 0.1 | |
| G012401 | 46.56 | 97.2 | 0.1 | 2.42 | G012401 | 46.56 | 94.2 | 5.2 | 2.36 |
| | 15.52 | 95.0 | 1.4 | | | 15.52 | 92.1 | 1.6 | |
| | 5.17 | 82.3 | 2.8 | | | 5.17 | 77.8 | 4.7 | |
| | 1.72 | 31.8 | 0.6 | | | 1.72 | 33.6 | 1.1 | |
| | 0.57 | 8.2 | 0.8 | | | 0.57 | 9.0 | 1.5 | |
| | 0.19 | 1.8 | 0.5 | | | 0.19 | 1.7 | 0.2 | |
| | 0.06 | 0.1 | 0.1 | | | 0.06 | 0.9 | 0.1 | |
| | 0.02 | 0.2 | 0.1 | | | 0.02 | 0.2 | 0.1 | |
| G018804 | 46.56 | 93.2 | 5.9 | 2.87 | G018811 | 46.56 | 90.8 | 3.3 | 2.91 |
| | 15.52 | 92.2 | 5.4 | | | 15.52 | 92.5 | 4.5 | |
| | 5.17 | 75.2 | 2.3 | | | 5.17 | 69.8 | 7.4 | |
| | 1.72 | 22.0 | 1.6 | | | 1.72 | 24.8 | 3.4 | |
| | 0.57 | 4.3 | 0.9 | | | 0.57 | 3.9 | 0.4 | |
| | 0.19 | 0.7 | 0.4 | | | 0.19 | 0.6 | 0.2 | |
| | 0.06 | 0.2 | 0.0 | | | 0.06 | 0.2 | 0.1 | |
| | 0.02 | 0.2 | 0.1 | | | 0.02 | 0.2 | 0.1 | |
| G018805 | 46.56 | 93.9 | 2.7 | 3.00 | G018812 | 46.56 | 93.4 | 3.2 | 2.81 |
| | 15.52 | 96.6 | 0.8 | | | 15.52 | 87.5 | 0.4 | |
| | 5.17 | 74.8 | 7.6 | | | 5.17 | 72.4 | 3.9 | |
| | 1.72 | 21.4 | 1.2 | | | 1.72 | 24.7 | 2.5 | |
| | 0.57 | 4.0 | 1.2 | | | 0.57 | 3.8 | 0.7 | |
| | 0.19 | 0.5 | 0.1 | | | 0.19 | 0.8 | 0.4 | |
| | 0.06 | 0.2 | 0.0 | | | 0.06 | 0.4 | 0.4 | |
| | 0.02 | 0.2 | 0.0 | | | 0.02 | 0.3 | 0.1 | |
| G018806 | 46.56 | 93.0 | 0.4 | 1.60 | G018813 | 46.56 | 94.6 | 1.5 | 1.73 |
| | 15.52 | 93.2 | 4.6 | | | 15.52 | 91.1 | 4.3 | |
| | 5.17 | 87.1 | 4.6 | | | 5.17 | 84.2 | 1.5 | |
| | 1.72 | 49.1 | 2.7 | | | 1.72 | 45.6 | 1.4 | |
| | 0.57 | 15.8 | 1.6 | | | 0.57 | 16.3 | 0.8 | |
| | 0.19 | 3.6 | 0.3 | | | 0.19 | 3.5 | 0.6 | |
| | 0.06 | 0.7 | 0.2 | | | 0.06 | 0.7 | 0.3 | |
| | 0.02 | 0.2 | 0.1 | | | 0.02 | 0.3 | 0.0 | |
| G018807 | 46.56 | 94.3 | 4.8 | 2.21 | G018814 | 46.56 | 95.3 | 1.0 | 1.72 |
| | 15.52 | 92.3 | 3.5 | | | 15.52 | 92.7 | 0.3 | |
| | 5.17 | 83.6 | 2.8 | | | 5.17 | 85.0 | 2.5 | |
| | 1.72 | 34.4 | 4.4 | | | 1.72 | 47.2 | 2.3 | |
| | 0.57 | 8.1 | 0.4 | | | 0.57 | 12.8 | 4.5 | |
| | 0.19 | 2.4 | 0.1 | | | 0.19 | 1.9 | 0.2 | |
| | 0.06 | 0.3 | 0.1 | | | 0.06 | 0.4 | 0.2 | |
| | 0.02 | 0.8 | 0.5 | | | 0.02 | 0.2 | 0.0 | |
| G018808 | 46.56 | 96.2 | 0.5 | 4.21 | G018815 | 46.56 | 92.8 | 3.3 | 2.57 |
| | 15.52 | 90.9 | 4.8 | | | 15.52 | 91.8 | 4.7 | |
| | 5.17 | 58.9 | 1.1 | | | 5.17 | 74.0 | 3.7 | |
| | 1.72 | 13.0 | 4.9 | | | 1.72 | 30.6 | 0.8 | |
| | 0.57 | 2.7 | 0.6 | | | 0.57 | 7.4 | 1.6 | |
| | 0.19 | 0.3 | 0.0 | | | 0.19 | 1.7 | 0.5 | |
| | 0.06 | 0.3 | 0.1 | | | 0.06 | 0.6 | 0.1 | |
| | 0.02 | 0.2 | 0.1 | | | 0.02 | 0.2 | 0.1 | |
| G018809 | 46.56 | 90.0 | 2.6 | 2.84 | G018816 | 46.56 | 87.6 | 2.1 | 4.03 |
| | 15.52 | 90.8 | 2.7 | | | 15.52 | 89.7 | 1.0 | |
| | 5.17 | 70.6 | 2.1 | | | 5.17 | 57.6 | 6.3 | |
| | 1.72 | 25.0 | 1.4 | | | 1.72 | 11.8 | 0.5 | |
| | 0.57 | 5.2 | 0.5 | | | 0.57 | 1.6 | 0.4 | |
| | 0.19 | 1.3 | 0.3 | | | 0.19 | 0.4 | 0.3 | |
| | 0.06 | 0.2 | 0.0 | | | 0.06 | 0.2 | 0.1 | |
| | 0.02 | 0.3 | 0.1 | | | 0.02 | 0.2 | 0.1 | |
| G018810 | 46.56 | 94.1 | 0.1 | 2.36 | | | | | |
| | 15.52 | 91.4 | 3.7 | | | | | | |
| | 5.17 | 81.8 | 8.1 | | | | | | |
| | 1.72 | 30.8 | 7.4 | | | | | | |
| | 0.57 | 8.9 | 1.4 | | | | | | |
| | 0.19 | 1.4 | 0.7 | | | | | | |
| | 0.06 | 0.5 | 0.3 | | | | | | |
| | 0.02 | 0.2 | 0.1 | | | | | | |

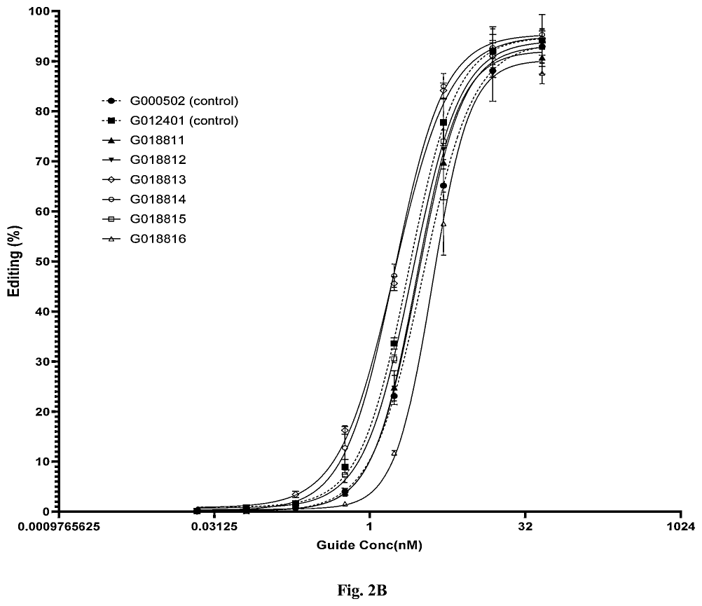

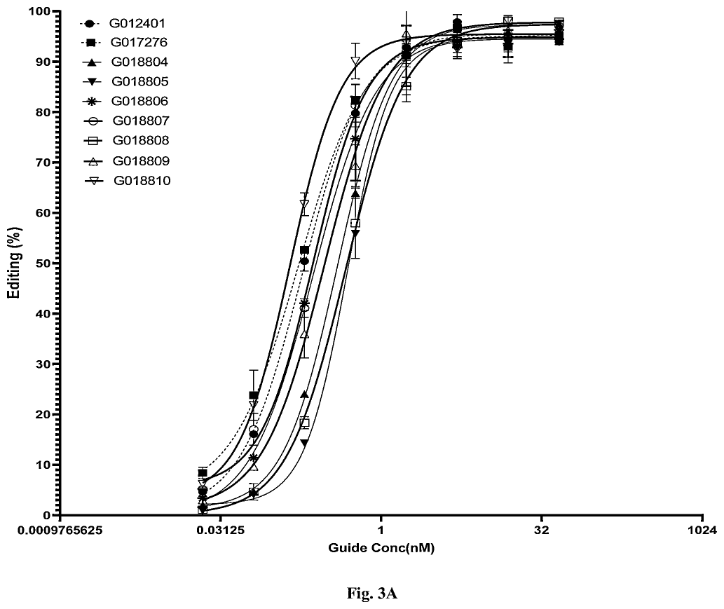

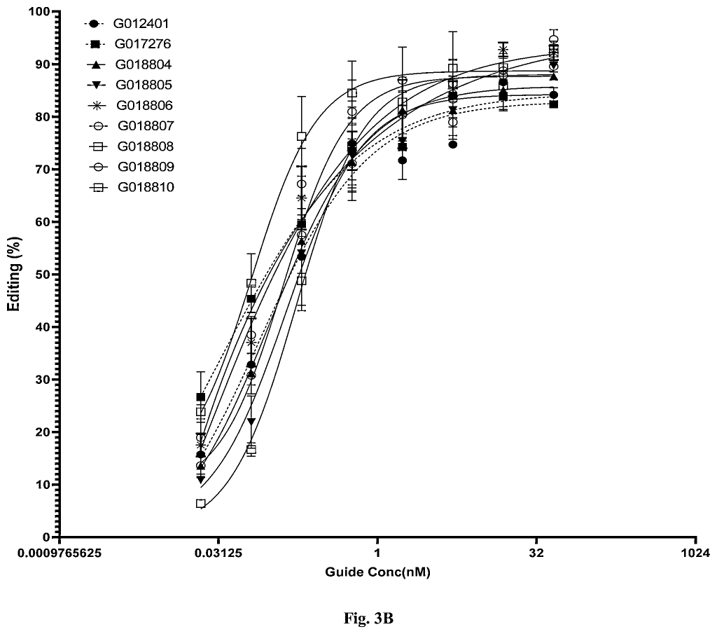

Selected sgRNAs from Table 7 were further evaluated in primary mouse hepatocytes (PMH) and primary cynomolgus hepatocytes (PCH) using the same methods to prepare, treat by LNP, and analyze cells described above. PMH (Lot #839) and PCH (Lot #CY6011) cells from Gibco™ were used and plated at a density of 15,000 and 30,000 cells/well, respectively. LNP formulations were prepared as described in Example 1 at a molar ratio of 50% Lipid A, 38% cholesterol, 9% DSPC, and 3% PEG2k-DMG, using an N:P molar ratio of about 6 and a 1:2 ratio of gRNA:mRNA by weight. Guides were assayed in an 8 point 3-fold dilution dose response curve starting at 46.5 nM guide concentration as shown in Table 8. Samples were run in duplicate. EC₅₀ values and mean editing results for PMH and PCH are shown in Table 8. Dose response curves are plotted in FIG. 3A and FIG. 3B, respectively. EC₅₀ values that were cited as undefined are denoted with “ND”.

TABLE 8
Editing in PMH and PCH

| | PMH | | | | PCH | | | |
|---|---|---|---|---|---|---|---|---|
| | Guide conc. | Mean | | | Guide conc. | Mean | | |
| Guide ID | (nM) | % Edit | SD | EC50 | (nM) | % Edit | SD | EC50 |
| G012401 | 46.56 | 94.0 | 0.5 | 0.18 | 46.56 | 84.2 | 1.3 | 0.07 |
| | 15.52 | 94.4 | 4.7 | | 15.52 | 86.6 | 5.4 | |
| | 5.17 | 97.9 | 0.1 | | 5.17 | 74.7 | 0.7 | |
| | 1.72 | 92.8 | 0.1 | | 1.72 | 71.7 | 3.7 | |
| | 0.57 | 79.8 | 1.8 | | 0.57 | 74.9 | 6.9 | |
| | 0.19 | 50.4 | 2.0 | | 0.19 | 53.4 | 3.9 | |
| | 0.06 | 16.1 | 0.4 | | 0.06 | 32.9 | 0.5 | |
| | 0.02 | 4.7 | 0.9 | | 0.02 | 15.8 | 3.7 | |
| G017276 | 46.56 | 95.3 | 0.5 | 0.17 | 46.56 | 82.4 | 0.5 | 0.03 |
| | 15.52 | 93.0 | 0.4 | | 15.52 | 83.8 | 2.4 | |
| | 5.17 | 96.7 | 1.4 | | 5.17 | 84.0 | 0.6 | |
| | 1.72 | 91.2 | 9.1 | | 1.72 | 74.2 | 2.5 | |
| | 0.57 | 82.2 | 3.3 | | 0.57 | 73.5 | 6.4 | |
| | 0.19 | 52.7 | 0.1 | | 0.19 | 59.6 | 0.6 | |
| | 0.06 | 23.8 | 4.9 | | 0.06 | 45.4 | 2.6 | |
| | 0.02 | 8.4 | 1.1 | | 0.02 | 26.7 | 4.8 | |
| G018804 | 46.56 | 97.5 | 0.7 | 0.39 | 46.56 | 87.7 | 0.2 | 0.09 |
| | 15.52 | 95.9 | 3.3 | | 15.52 | 85.1 | 3.2 | |
| | 5.17 | 96.5 | 2.9 | | 5.17 | 81.3 | 6.4 | |
| | 1.72 | 92.0 | 2.3 | | 1.72 | 81.3 | 6.3 | |
| | 0.57 | 63.9 | 1.0 | | 0.57 | 71.4 | 7.4 | |
| | 0.19 | 24.1 | 0.1 | | 0.19 | 56.4 | 6.2 | |
| | 0.06 | 4.7 | 0.3 | | 0.06 | 31.2 | 3.9 | |
| | 0.02 | 2.0 | 0.3 | | 0.02 | 13.6 | 2.3 | |
| G018805 | 46.56 | 94.9 | 1.5 | 0.48 | 46.56 | 89.7 | 0.7 | 0.13 |
| | 15.52 | 93.5 | 0.7 | | 15.52 | 83.9 | 0.1 | |
| | 5.17 | 92.7 | 0.1 | | 5.17 | 81.3 | 1.6 | |
| | 1.72 | 91.2 | 2.2 | | 1.72 | 75.3 | 0.8 | |
| | 0.57 | 55.9 | 0.2 | | 0.57 | 72.7 | 7.0 | |
| | 0.19 | 14.3 | 0.7 | | 0.19 | 54.0 | 10.9 | |
| | 0.06 | 4.1 | 0.1 | | 0.06 | 21.9 | 4.9 | |
| | 0.02 | 0.9 | 0.2 | | 0.02 | 10.9 | 0.3 | |
| G018806 | 46.56 | 96.3 | 1.8 | 0.23 | 46.56 | 92.2 | 1.3 | 0.02 |
| | 15.52 | 93.6 | 2.7 | | 15.52 | 92.8 | 1.3 | |
| | 5.17 | 94.1 | 1.2 | | 5.17 | 85.3 | 1.4 | |
| | 1.72 | 91.2 | 6.0 | | 1.72 | 80.5 | 6.5 | |
| | 0.57 | 74.7 | 8.3 | | 0.57 | 73.5 | 3.7 | |
| | 0.19 | 42.1 | 0.8 | | 0.19 | 64.6 | 6.0 | |
| | 0.06 | 11.4 | 0.7 | | 0.06 | 37.1 | 4.6 | |
| | 0.02 | 3.4 | 1.8 | | 0.02 | 17.6 | 2.2 | |
| G018807 | 46.56 | 97.1 | 0.6 | 0.23 | 46.56 | 94.7 | 1.8 | ND |
| | 15.52 | 93.2 | 1.3 | | 15.52 | 88.8 | 2.3 | |
| | 5.17 | 93.1 | 1.3 | | 5.17 | 79.0 | 0.9 | |
| | 1.72 | 91.9 | 1.7 | | 1.72 | 80.4 | 4.3 | |
| | 0.57 | 81.3 | 4.2 | | 0.57 | 71.1 | 4.7 | |
| | 0.19 | 41.1 | 1.8 | | 0.19 | 67.2 | 6.8 | |
| | 0.06 | 17.1 | 3.2 | | 0.06 | 38.5 | 3.5 | |
| | 0.02 | 5.2 | 0.1 | | 0.02 | 19.0 | 2.9 | |
| G018808 | 46.56 | 98.0 | 0.5 | 0.47 | 46.56 | 93.0 | 0.8 | 0.1 |
| | 15.52 | 98.1 | 0.1 | | 15.52 | 85.9 | 0.6 | |
| | 5.17 | 94.1 | 3.5 | | 5.17 | 86.3 | 9.9 | |
| | 1.72 | 85.2 | 1.8 | | 1.72 | 81.5 | 0.5 | |
| | 0.57 | 58.0 | 7.0 | | 0.57 | 74.5 | 8.6 | |
| | 0.19 | 18.4 | 1.2 | | 0.19 | 48.8 | 4.7 | |
| | 0.06 | 4.7 | 1.6 | | 0.06 | 16.7 | 1.3 | |
| | 0.02 | 1.1 | 0.1 | | 0.02 | 6.4 | 0.3 | |
| G018809 | 46.56 | 96.2 | 1.8 | 0.3 | 46.56 | 89.6 | 1.1 | 0.12 |
| | 15.52 | 96.3 | 1.3 | | 15.52 | 87.9 | 0.9 | |
| | 5.17 | 97.2 | 2.1 | | 5.17 | 83.3 | 7.6 | |
| | 1.72 | 95.6 | 1.6 | | 1.72 | 86.9 | 6.4 | |
| | 0.57 | 69.4 | 4.2 | | 0.57 | 81.0 | 6.0 | |
| | 0.19 | 36.0 | 4.7 | | 0.19 | 57.5 | 3.8 | |
| | 0.06 | 9.7 | 0.6 | | 0.06 | 30.8 | 1.8 | |
| | 0.02 | 3.0 | 0.1 | | 0.02 | 13.7 | 0.1 | |
| G018810 | 46.56 | 95.7 | 0.4 | 0.14 | 46.56 | 92.1 | 2.3 | 0.06 |
| | 15.52 | 98.1 | 0.2 | | 15.52 | 89.4 | 2.2 | |
| | 5.17 | 92.4 | 1.4 | | 5.17 | 89.3 | 1.5 | |
| | 1.72 | 93.8 | 1.2 | | 1.72 | 82.8 | 2.1 | |
| | 0.57 | 90.1 | 3.5 | | 0.57 | 84.5 | 6.1 | |
| | 0.19 | 61.7 | 2.3 | | 0.19 | 76.3 | 7.6 | |
| | 0.06 | 21.9 | 0.1 | | 0.06 | 48.4 | 5.6 | |
| | 0.02 | 6.2 | 0.0 | | 0.02 | 23.9 | 1.3 | |

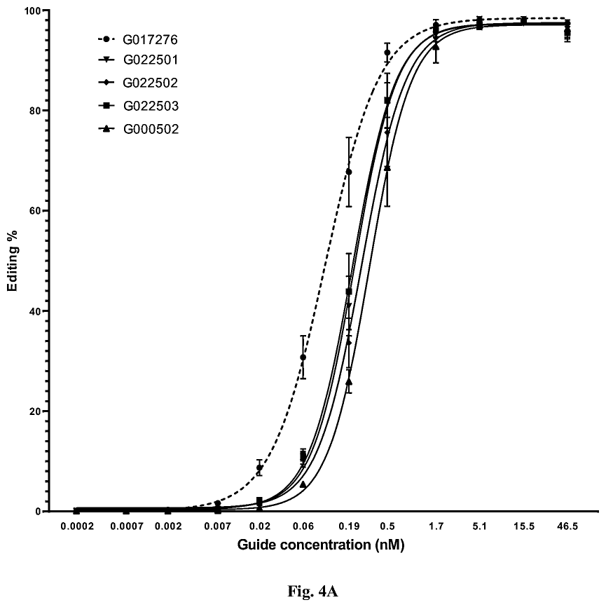

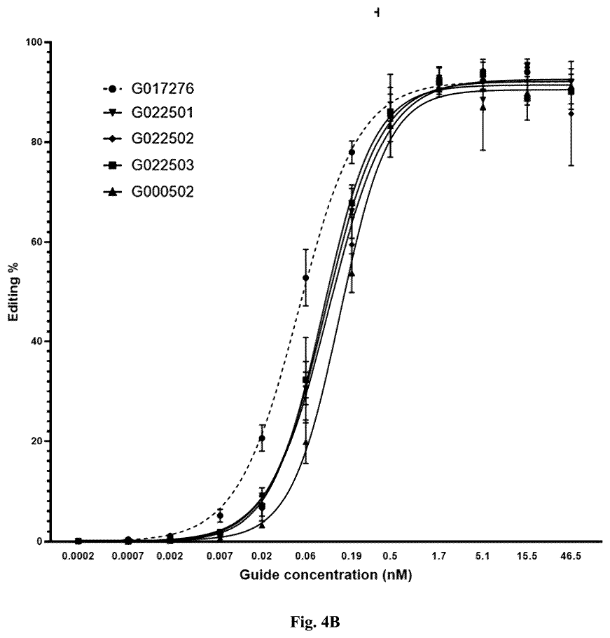

Additional sgRNAs were evaluated in primary mouse hepatocytes (PMH), primary rat hepatocytes (PRH), and primary cynomolgus hepatocytes (PCH) using the same methods to prepare, treat by LNP, and analyze cells described above unless otherwise noted. PMH (Lot #839) cells were used and plated at a density of 15,000 cells/well. PMH (Lot #839 or Lot #mc114), PCH (Lot #10136011), and PRH (Lot #977A) cells were used and plated at densities of 15,000, 33,000, and 30,000 cells/well, respectively. respectively. LNP formulations were prepared as described in Example 1 at a molar ratio of 50% Lipid A, 38% cholesterol, 9% DSPC, and 3% PEG2k-DMG, using an N:P molar ratio of about 6 and a 1:2 ratio of gRNA:mRNA by weight. Guides were assayed in a 12 point 3-fold dose response curve starting at 46.5 nM guide concentration as shown in Tables 8 and 9. Controls, G017276 and G000502, for PMH and PCH were run with 6 and 4 replicates and remaining samples with 4 and 2 replicates, respectively. Controls, G017276 and G000502, for PMH and PCH were run with 6 and 3 replicates and test samples with 4 and 2 replicates, respectively. Controls, G018631 and G022500, for PRH were run with 4 replicates and remaining samples with 2 replicates. EC₅₀ values and mean editing results for PMH and PCH are shown in Table 9 and for PRH in Table 10. Dose response curves are plotted for PMH, PCH, and PRH in FIG. 4A, FIG. 4B, and FIG. 4C, respectively.

TABLE 9
Editing in PMH and PCH

| | PMH | | | | | PCH | | |
|---|---|---|---|---|---|---|---|---|
| | Guide conc. | Mean | | | Guide conc. | Mean | | |
| Guide ID | (nM) | % Edit | SD | EC50 | (nM) | % Edit | SD | EC50 |
| G017276 | 46.5 | 97.4 | 0.3 | 0.11 | 46.5 | 90.6 | 4.0 | 0.05 |
| | 15.5 | 98.0 | 0.4 | | 15.5 | 94.1 | 1.8 | |
| | 5.1 | 98.1 | 0.4 | | 5.1 | 92.4 | 2.1 | |
| | 1.7 | 97.1 | 1.1 | | 1.7 | 92.8 | 2.3 | |
| | 0.5 | 91.5 | 1.9 | | 0.5 | 85.3 | 8.4 | |
| | 0.19 | 67.7 | 6.9 | | 0.19 | 78.0 | 2.3 | |
| | 0.06 | 30.8 | 4.3 | | 0.06 | 52.8 | 5.7 | |
| | 0.02 | 8.7 | 1.6 | | 0.02 | 20.6 | 2.7 | |
| | 0.007 | 1.6 | 0.5 | | 0.007 | 5.1 | 1.3 | |
| | 0.002 | 0.3 | 0.2 | | 0.002 | 1.1 | 0.5 | |
| | 0.0007 | 0.1 | 0.1 | | 0.0007 | 0.5 | 0.2 | |
| | 0.0002 | 0.1 | 0.0 | | 0.0002 | 0.1 | 0.1 | |
| G022502 | 46.5 | 96.2 | 1.3 | 0.22 | 46.5 | 92.1 | 1.6 | 0.1 |
| | 15.5 | 97.4 | 0.6 | | 15.5 | 95.2 | 1.4 | |
| | 5.1 | 97.6 | 1.1 | | 5.1 | 88.5 | 2.0 | |
| | 1.7 | 95.1 | 1.4 | | 1.7 | 90.4 | 0.0 | |
| | 0.5 | 82.1 | 3.4 | | 0.5 | 85.2 | 0.1 | |
| | 0.19 | 41.0 | 5.9 | | 0.19 | 66.1 | 5.3 | |
| | 0.06 | 10.9 | 1.5 | | 0.06 | 30.6 | 3.3 | |
| | 0.02 | 2.2 | 0.4 | | 0.02 | 9.1 | 1.6 | |
| | 0.007 | 0.4 | 0.1 | | 0.007 | 1.5 | 0.4 | |
| | 0.002 | 0.1 | 0.1 | | 0.002 | 0.3 | 0.3 | |
| | 0.0007 | 0.1 | 0.1 | | 0.0007 | 0.1 | 0.0 | |
| | 0.0002 | 0.1 | 0.0 | | 0.0002 | 0.1 | 0.1 | |
| G022503 | 46.5 | 95.8 | 1.5 | 0.26 | 46.5 | 85.7 | 10.5 | 0.11 |
| | 15.5 | 97.9 | 0.8 | | 15.5 | 95.0 | 0.8 | |
| | 5.1 | 97.5 | 0.1 | | 5.1 | 94.4 | 0.5 | |
| | 1.7 | 94.5 | 1.9 | | 1.7 | 92.0 | 2.9 | |
| | 0.5 | 75.6 | 6.8 | | 0.5 | 85.6 | 5.4 | |
| | 0.19 | 33.6 | 4.9 | | 0.19 | 59.5 | 6.0 | |
| | 0.06 | 10.4 | 1.6 | | 0.06 | 32.3 | 8.6 | |
| | 0.02 | 1.8 | 0.1 | | 0.02 | 6.5 | 2.4 | |
| | 0.007 | 0.3 | 0.1 | | 0.007 | 1.1 | 0.4 | |
| | 0.002 | 0.1 | 0.1 | | 0.002 | 0.4 | 0.4 | |
| | 0.0007 | 0.1 | 0.1 | | 0.0007 | 0.0 | 0.0 | |
| | 0.0002 | 0.1 | 0.1 | | 0.0002 | 0.1 | 0.0 | |
| G022501 | 46.5 | 95.5 | 1.8 | 0.21 | 46.5 | 90.2 | 2.5 | 0.9 |
| | 15.5 | 97.8 | 0.5 | | 15.5 | 88.8 | 4.4 | |
| | 5.1 | 97.2 | 1.0 | | 5.1 | 93.6 | 3.0 | |
| | 1.7 | 95.9 | 1.3 | | 1.7 | 92.0 | 1.4 | |
| | 0.5 | 82.0 | 5.4 | | 0.5 | 86.2 | 1.9 | |
| | 0.19 | 43.9 | 7.6 | | 0.19 | 67.8 | 2.9 | |
| | 0.06 | 10.9 | 0.8 | | 0.06 | 32.4 | 3.6 | |
| | 0.02 | 2.0 | 0.8 | | 0.02 | 7.1 | 2.1 | |
| | 0.007 | 0.5 | 0.1 | | 0.007 | 1.9 | 0.6 | |
| | 0.002 | 0.1 | 0.0 | | 0.002 | 0.2 | 0.3 | |
| | 0.0007 | 0.1 | 0.1 | | 0.0007 | 0.1 | 0.1 | |
| | 0.0002 | 0.1 | 0.0 | | 0.0002 | 0.1 | 0.0 | |
| G022504 | 46.5 | 96.3 | 1.7 | 0.32 | 46.5 | 91.3 | 1.1 | 0.14 |
| | 15.5 | 97.7 | 0.3 | | 15.5 | 90.0 | 2.5 | |
| | 5.1 | 97.0 | 0.7 | | 5.1 | 87.2 | 8.8 | |
| | 1.7 | 92.8 | 3.3 | | 1.7 | 90.6 | 1.0 | |
| | 0.5 | 68.7 | 7.8 | | 0.5 | 83.3 | 6.4 | |
| | 0.19 | 25.9 | 2.3 | | 0.19 | 53.8 | 3.9 | |
| | 0.06 | 5.4 | 0.1 | | 0.06 | 19.9 | 4.4 | |
| | 0.02 | 0.7 | 0.1 | | 0.02 | 3.3 | 0.6 | |
| | 0.007 | 0.2 | 0.1 | | 0.007 | 0.7 | 0.1 | |
| | 0.002 | 0.1 | 0.0 | | 0.002 | 0.2 | 0.0 | |
| | 0.0007 | 0.1 | 0.0 | | 0.0007 | 0.2 | 0.1 | |
| | 0.0002 | 0.1 | 0.0 | | 0.0002 | 0.1 | 0.0 | |
| G000502 | 46.5 | 95.4 | 1.0 | 0.34 | 46.5 | 88.5 | 6.3 | 0.2 |
| | 15.5 | 97.3 | 0.4 | | 15.5 | 93.3 | 1.9 | |
| | 5.1 | 96.5 | 1.8 | | 5.1 | 93.0 | 1.5 | |
| | 1.7 | 90.5 | 4.8 | | 1.7 | 88.2 | 3.3 | |
| | 0.5 | 64.1 | 8.3 | | 0.5 | 75.4 | 1.6 | |
| | 0.19 | 24.8 | 3.6 | | 0.19 | 42.0 | 4.0 | |
| | 0.06 | 5.4 | 0.4 | | 0.06 | 14.1 | 2.4 | |
| | 0.02 | 1.2 | 0.3 | | 0.02 | 3.0 | 0.3 | |
| | 0.007 | 0.3 | 0.1 | | 0.007 | 0.6 | 0.1 | |
| | 0.002 | 0.1 | 0.1 | | 0.002 | 0.2 | 0.1 | |
| | 0.0007 | 0.1 | 0.0 | | 0.0007 | 0.0 | 0.1 | |
| | 0.0002 | 0.1 | 0.0 | | 0.0002 | 0.1 | 0.1 | |

TABLE 10
Editing in PRH

| | Guide conc. | Mean | | |
|---|---|---|---|---|
| Guide ID | (nM) | % Edit | SD | EC50 |
| G018631 | 46.5 | 99.0 | 0.2 | 0.02 |
| | 15.5 | 99.6 | 0.1 | |
| | 5.1 | 99.5 | 0.2 | |
| | 1.7 | 99.0 | 0.2 | |
| | 0.5 | 97.2 | 0.7 | |
| | 0.19 | 92.3 | 1.3 | |
| | 0.06 | 78.6 | 4.0 | |
| | 0.02 | 49.9 | 8.5 | |
| | 0.007 | 15.5 | 3.6 | |
| | 0.002 | 4.0 | 1.4 | |
| | 0.0007 | 0.9 | 0.4 | |
| | 0.0002 | 0.4 | 0.1 | |
| G022499 | 46.5 | 98.3 | 0.4 | 0.06 |
| | 15.5 | 99.2 | 0.7 | |
| | 5.1 | 99.3 | 0.3 | |
| | 1.7 | 97.7 | 0.0 | |
| | 0.5 | 94.4 | 0.8 | |
| | 0.19 | 81.7 | 2.9 | |
| | 0.06 | 49.6 | 5.0 | |
| | 0.02 | 16.5 | 4.2 | |
| | 0.007 | 3.3 | 1.9 | |
| | 0.002 | 0.9 | 0.4 | |
| | 0.0007 | 0.2 | 0.0 | |
| | 0.0002 | 0.2 | 0.1 | |
| G022497 | 46.5 | 98.3 | 0.4 | 0.07 |
| | 15.5 | 98.9 | 0.4 | |
| | 5.1 | 99.4 | 0.1 | |
| | 1.7 | 97.9 | 1.1 | |
| | 0.5 | 94.0 | 2.9 | |
| | 0.19 | 82.4 | 5.9 | |
| | 0.06 | 41.6 | | |
| | 0.02 | 16.7 | 7.1 | |
| | 0.007 | 3.1 | 1.8 | |
| | 0.002 | 0.4 | 0.1 | |
| | 0.0007 | 0.4 | 0.0 | |
| | 0.0002 | 0.1 | 0.0 | |
| G022498 | 46.5 | 97.9 | 0.0 | 0.04 |
| | 15.5 | 99.1 | 0.4 | |
| | 5.1 | 99.2 | 0.0 | |
| | 1.7 | 98.7 | 0.1 | |
| | 0.5 | 96.0 | 1.5 | |
| | 0.19 | 89.6 | 2.3 | |
| | 0.06 | 65.9 | 10.2 | |
| | 0.02 | 31.9 | 10.0 | |
| | 0.007 | 7.4 | 2.3 | |
| | 0.002 | 1.5 | 0.7 | |
| | 0.0007 | 0.4 | 0.1 | |
| | 0.0002 | 0.1 | 0.0 | |
| G00534 | 46.5 | 98.4 | 0.4 | 0.08 |
| | 15.5 | 99.0 | 0.4 | |
| | 5.1 | 99.0 | 0.5 | |
| | 1.7 | 97.9 | 0.8 | |
| | 0.5 | 92.1 | | |
| | 0.19 | 77.5 | 7.1 | |
| | 0.06 | 40.3 | 7.0 | |
| | 0.02 | 11.8 | 4.2 | |
| | 0.007 | 2.2 | 0.8 | |
| | 0.002 | 0.3 | 0.0 | |
| | 0.0007 | 0.2 | 0.1 | |
| | 0.0002 | 0.2 | 0.1 | |
| G022500 | 46.5 | 98.4 | 0.4 | 0.07 |
| | 15.5 | 99.1 | 0.2 | |
| | 5.1 | 99.3 | 0.3 | |
| | 1.7 | 97.7 | 0.7 | |
| | 0.5 | 93.3 | 1.8 | |
| | 0.19 | 78.1 | 4.8 | |
| | 0.06 | 44.3 | 6.1 | |
| | 0.02 | 12.9 | 3.1 | |
| | 0.007 | 2.4 | 0.8 | |
| | 0.002 | 0.4 | 0.2 | |
| | 0.0007 | 0.2 | 0.1 | |
| | 0.0002 | 0.1 | 0.1 | |

### Example 4. In Vivo Editing in Mouse Liver Using Lipid Nanoparticles (LNPs)

The LNPs used in all in vivo studies were formulated as described in Example 1. Deviations from the protocol are noted in the respective Example. Transport and storage solution (TSS) used in LNP preparation was dosed in the experiment as a vehicle-only negative control. The nucleotide sequences of the sgRNA contained in the LNPs all target the same sequence in the TTR gene as indicated in Tables 2A-2B.

### Example 4.1 In Vivo Editing in the Mouse Model

Selected guide designs from Tables 2A-2B were tested for editing efficiency in vivo. CD-1 female mice, ranging 6-10 weeks of age were used in each study involving mice. Animals were weighed pre-dose. LNPs were dosed via the lateral tail vein at a volume of 0.2 mL per animal (approximately 10 mL per kilogram body weight). The animals were observed at approximately 6 hours post dose for adverse effects. Body weight was measured at twenty-four hours post-administration, and animals were euthanized at 8 days post dose by exsanguination under isoflurane anesthesia. Blood was collected via cardiac puncture into serum separator tubes or into tubes containing buffered sodium citrate for plasma as described herein. For studies involving in vivo editing, liver tissue was collected from the left medial lobe from each animal for DNA extraction and analysis.

For the in vivo studies, genomic DNA was extracted from 10 mg of tissue using a bead-based extraction kit, e.g. the Zymo Quick-DNA 96 kit (Zymo Research, Cat. #D3010) according to the manufacturer's protocol, which includes homogenizing the tissue in lysis buffer (approximately 400 μL/10 mg tissue). All DNA samples were normalized to 100 ng/μL concentration for PCR and subsequent NGS analysis, as described in Example 1.

### Example 4.2 Transthyretin (TTR) ELISA Analysis Used in Animal Studies

Blood was collected, and the serum was isolated as described above. The total TTR serum levels were determined using a Mouse Prealbumin (Transthyretin) ELISA Kit (Aviva Systems Biology, Cat. OKIA00111); rat TTR serum levels were measured using a rat specific ELISA kit (Aviva Systems Biology catalog number OKIA00159). Kit reagents and standards were prepared according to the manufacturer's protocol. Mouse or rat serum was diluted to a final dilution of 10,000-fold with 1× assay diluent. This was done by carrying out two sequential 50-fold dilutions resulting in a 2500-fold dilution. A final 4-fold dilution step was carried out for a total sample dilution of 10,000-fold. Both standard curve dilutions (100 μL each) and diluted serum samples were added to each well of the ELISA plate pre-coated with capture antibody. The plate was incubated at room temperature for 30 minutes before washing. Enzyme-antibody conjugate (100 μL per well) was added for a 20-minute incubation. Unbound antibody conjugate was removed and the plate was washed again before the addition of the chromogenic substrate solution. The plate was incubated for 10 minutes before adding 100 μL of the stop solution, e.g., sulfuric acid (approximately 0.3 M). The plate was read on a SpectraMax M5 or Clariostar plate reader at an absorbance of 450 nm. Serum TTR levels were calculated by SoftMax Pro software ver. 6.4.2 or Mars software ver. 3.31 using a four-parameter logistic curve fit off the standard curve. Final serum values were adjusted for the assay dilution. Percent protein knockdown (% KD) values were determined relative to controls, which generally were animals sham-treated with vehicle (TSS) unless otherwise indicated. Negative % KD values were observed in individual or a group of animals with TTR levels higher than the control group average resulting in a negative knockdown value.

### Example 4.3 In Vivo Editing and Serum TTR Knockdown

LNPs were generally prepared as described in Example 1. LNP formulations were analyzed for average particle size, polydispersity (pdi), total RNA content and encapsulation efficiency of RNA as described in Example 1 and results shown in Table 11.

TABLE 11
LNP formulation analysis

| | | | Z-Ave | | Num Ave |
|---|---|---|---|---|---|
| LNP | Guide | Encapsulation | Size | | Size |
| ID | ID | (%) | (nm) | PDI | (nm) |
| LNP1 | TSS | 98 | 88 | 0.04 | 71 |
| LNP2 | G017276 | 99 | 89 | 0.02 | 71 |
| LNP3 | G018804 | 98 | 90 | 0.04 | 71 |
| LNP4 | G018805 | 98 | 92 | 0.02 | 75 |
| LNP5 | G018806 | 98 | 91 | 0 | 75 |
| LNP6 | G018807 | 98 | 90 | 0.02 | 73 |
| LNP7 | G018808 | 98 | 89 | 0.01 | 72 |
| LNP8 | G018809 | 98 | 87 | 0.04 | 68 |
| LNP9 | G018810 | 99 | 88 | 0.06 | 67 |

LNPs containing sgRNAs indicated in Table 12 were administered to female CD-1 mice (n=5) at a dose of 0.1 mg/kg of total RNA as described above. Guide G017276 served as the control. The editing efficiency, TTR protein levels, and percent TTR knockdown (% KD) for LNPs containing the indicated sgRNAs are shown in Table 12 and editing efficiency and TTR protein levels are illustrated in FIGS. 5A and 5B.

TABLE 12
Liver Editing, Serum TTR protein, and TTR protein knockdown

| | | | Mean | | | |
|---|---|---|---|---|---|---|
| | | | serum | | | |
| | Mean | | TTR | | Mean | |
| Guide ID | % indel | SD | (ug/ml) | SD | % KD | SD |
| TSS | 0.06 | 0.05 | 614.6 | 137.4 | 0 | 22.35 |
| G017276 | 64.12 | 5.97 | 107.2 | 38.65 | 82.57 | 6.29 |
| G018804 | 66.08 | 4.98 | 84.5 | 54.1 | 86.25 | 8.8 |
| G018805 | 59.88 | 4.28 | 118.8 | 59.23 | 80.67 | 9.64 |
| G018806 | 63.72 | 2.03 | 104.4 | 19.71 | 83.01 | 3.21 |
| G018807 | 65.34 | 6.11 | 86.4 | 43.46 | 85.94 | 7.07 |
| G018808 | 46.76 | 11.65 | 227.0 | 138.4 | 63.06 | 22.51 |
| G018809 | 64.74 | 2.8 | 78.2 | 20.79 | 87.28 | 3.38 |
| G018810 | 63.44 | 5.91 | 103.1 | 70.12 | 83.22 | 11.41 |

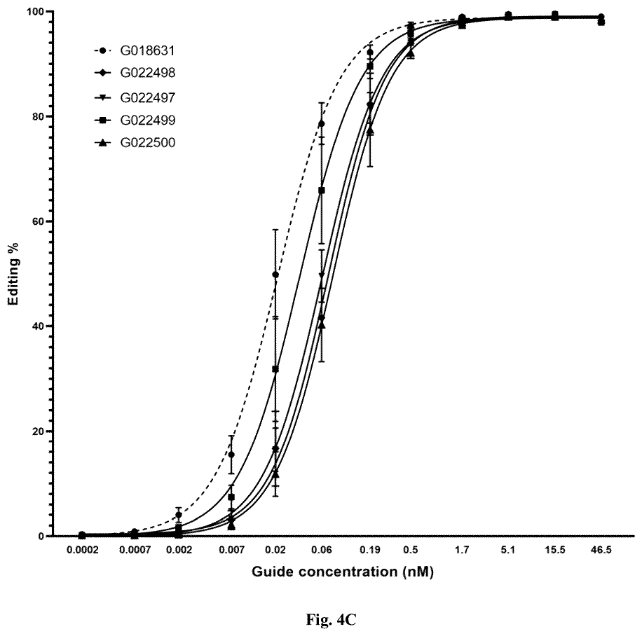

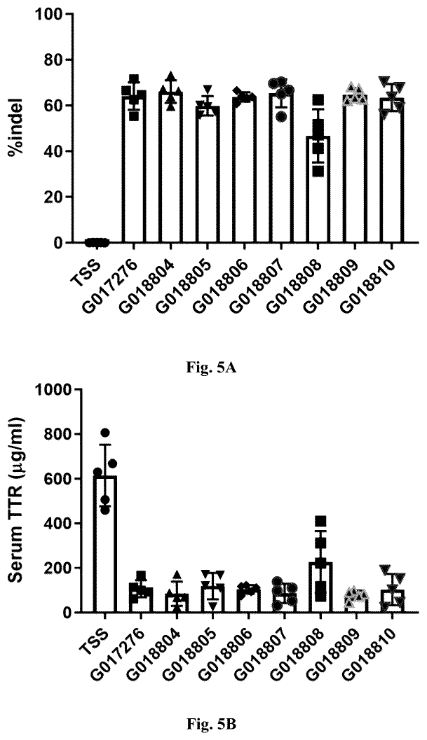

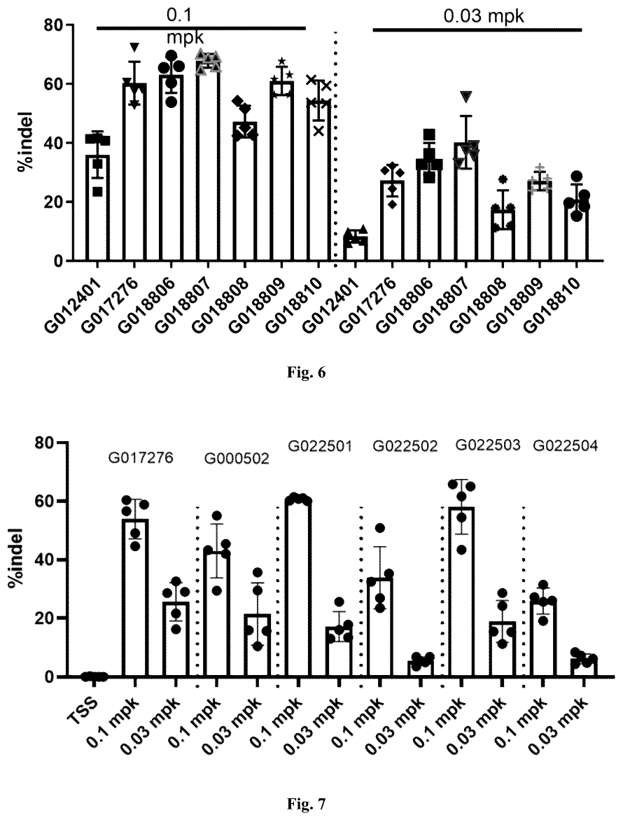

Selected guides from Table 12 were administered to female CD-1 mice (n=5) at 0.1 mg/kg and 0.03 mg/kg of total RNA as described above. Guides G0012401 and G001727 served as controls. Table 13 shows the editing efficiency, TTR protein levels, and percent TTR knockdown, respectively, for LNPs containing the indicated sgRNAs and editing efficiency is shown in FIG. 6.

TABLE 13
Liver Editing, Serum TTR protein, and % KD serum TTR protein

| | | | | Mean | | | |
|---|---|---|---|---|---|---|---|
| | Dose | Mean | | serum TTR | | Mean | |
| Guide | (mg/kg) | % indel | SD | (ug/ml) | SD | % KD | SD |
| TSS | 0 | 0.2 | 0 | 658.9 | 56.6 | 0 | 8.6 |
| G12401 | 0.1 | 36 | 7.9 | 324.0 | 115.0 | 50.8 | 17.5 |
| | 0.03 | 8.3 | 2.1 | 550.3 | 51.3 | 30.7 | 31.7 |
| G17276 | 0.1 | 60.3 | 7.3 | 115.1 | 54.2 | 68.4 | 31 |
| | 0.03 | 27.2 | 5.4 | 412.7 | 65.6 | 37.4 | 10 |
| G18806 | 0.1 | 63.1 | 6.1 | 89.4 | 76.2 | 86.4 | 11.6 |
| | 0.03 | 34.6 | 5.4 | 369.9 | 64.8 | 43.9 | 9.8 |
| G18807 | 0.1 | 67.8 | 2.3 | 75.5 | 14.8 | 88.6 | 2.3 |
| | 0.03 | 40.2 | 9 | 316.5 | 95.8 | 52 | 14.5 |
| G18808 | 0.1 | 47.3 | 5.4 | 202.2 | 35.8 | 69.3 | 5.4 |
| | 0.03 | 17.3 | 6.6 | 444.1 | 101.2 | 32.6 | 15.4 |
| G18809 | 0.1 | 61 | 4.8 | 114.0 | 44.7 | 82.7 | 6.8 |
| | 0.03 | 27 | 3.1 | 494.7 | 93.8 | 24.9 | 14.2 |
| G18810 | 0.1 | 54.4 | 6.7 | 156.2 | 81.0 | 76.3 | 12.3 |
| | 0.03 | 20.9 | 5.1 | 592.4 | 67.0 | 10.1 | 10.2 |

Further guides with the same targeting sequence with additional linker modifications were administered to female CD-1 mice (n=5) at 0.1 mg/kg and 0.03 mg/kg of total RNA as described above. Guides G017276 and G000502 served as controls. Table 14 shows the editing efficiency, TTR protein levels, and percent TTR knockdown, respectively, for LNPs containing the indicated sgRNAs and editing efficiency is shown in FIG. 7.

TABLE 14
Liver Editing, Serum TTR protein, and % KD serum TTR protein

| | Dose | Mean % | | Mean ug/ | | Mean | |
|---|---|---|---|---|---|---|---|
| Guide | (mg/kg) | Indel | SD | ml TTR | SD | % KD | SD |
| TSS | x | 0.14 | 0.09 | 588.8 | 62.3 | −6E−10 | 10.6 |
| G017276 | 0.1 | 53.92 | 6.78 | 154.5 | 77.0 | 73.8 | 13.1 |
| | 0.03 | 25.6 | 6.59 | 373.3 | 109.3 | 36.6 | 18.6 |
| G000502 | 0.1 | 43.04 | 9.20 | 240.5 | 52.1 | 59.2 | 8.8 |
| | 0.03 | 21.42 | 10.67 | 439.7 | 99.2 | 25.3 | 16.9 |
| G022501 | 0.1 | 60.74 | 0.59 | 116.0 | 26.6 | 80.3 | 4.5 |
| | 0.03 | 17.16 | 5.11 | 611.7 | 183.6 | −3.9 | 31.2 |
| G022502 | 0.1 | 33.82 | 10.61 | 472.2 | 205.4 | 19.8 | 34.9 |
| | 0.03 | 5.52 | 1.35 | 573.5 | 46.9 | 2.6 | 8.0 |
| G022503 | 0.1 | 58.1 | 9.36 | 154.2 | 78.5 | 73.8 | 13.3 |
| | 0.03 | 18.94 | 7.19 | 508.7 | 104.1 | 13.6 | 17.7 |
| G022504 | 0.1 | 25.88 | 4.45 | 527.5 | 122.2 | 10.4 | 20.8 |
| | 0.03 | | | 624.9 | 88.5 | −6.1 | 15.0 |

### Example 5. In Vivo Editing in Rat Liver Using Lipid Nanoparticles (LNPs)

### Example 5.1 LNP Delivery in Rat

Selected guide designs were further tested in rats. Sprague Dawley female rats from Charles River, ranging 6-8 weeks of age, were used in each study involving rats. LNPs were dosed via lateral tail vein injection. LNP formulations were prepared as described in Example 1 at a molar ratio of 50% Lipid A, 38% cholesterol, 9% DSPC, and 3% PEG2k-DMG, using an N:P molar ratio of about 6 and a 1:2 ratio of gRNA:mRNA by weight. The animals were observed post dose for adverse effects. Body weight was measured at twenty-four hours post-administration and animals euthanized post dose via exsanguination under CO₂ asphyxiation. Blood was collected via cardiac puncture into serum separator tubes (Geriner Bio One, Catalog #450472). For studies involving in vivo editing, liver tissue was collected from each animal. Genomic DNA was isolated and processed as described in Example 5. All DNA samples were prepared for PCR and subsequent NGS analysis as described in Example 5.

Editing efficiency in the liver and TTR serum protein levels were evaluated for each rat sample as described in Example 5. The results shown in each of the following study tables denote the sgRNA contained within each LNP (See Tables 2A-2B for sgRNA nucleotide sequences) which all target the same sequence in the TTR gene. LNPs were prepared as described in Example 5. Deviations from the protocol are noted in the respective Examples below.

### Example 5.2 In Vivo Editing and TTR Knockdown in the Rat Model

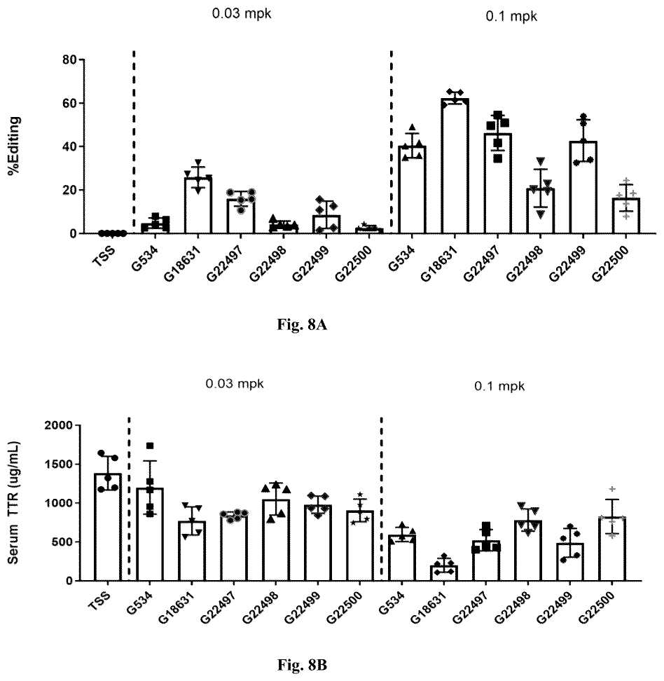

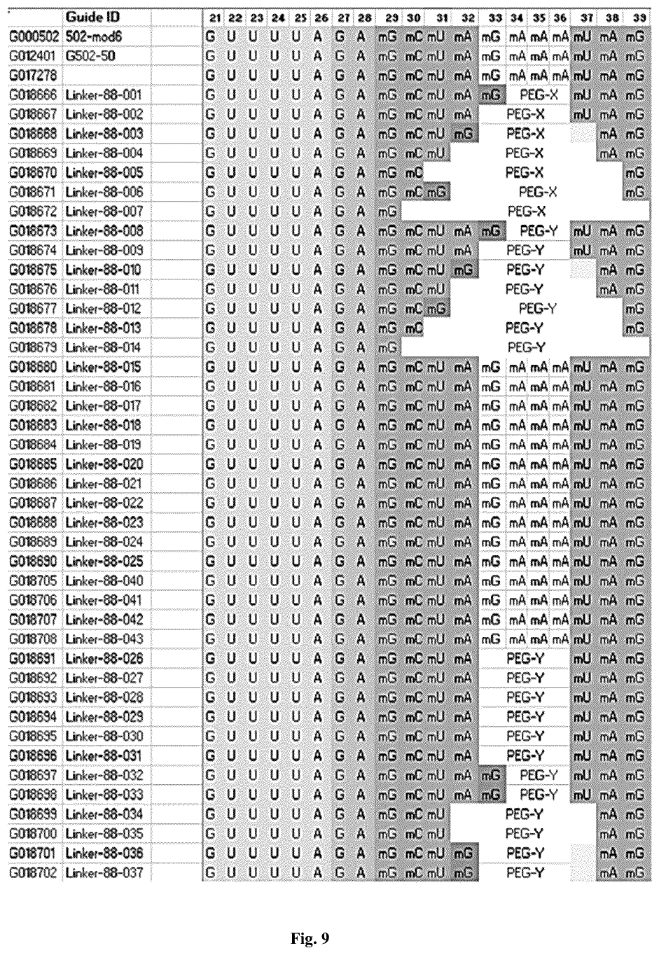

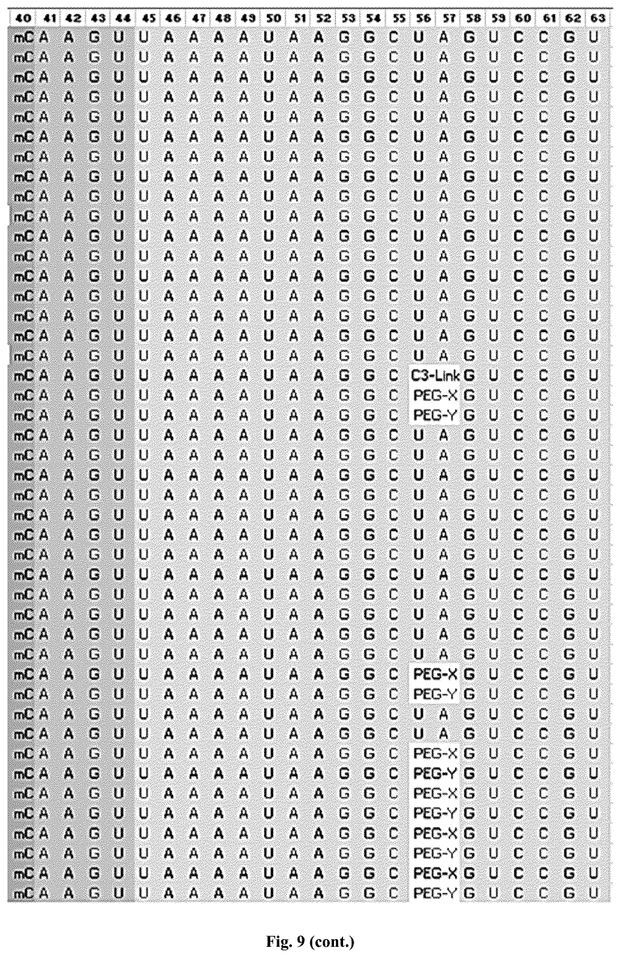

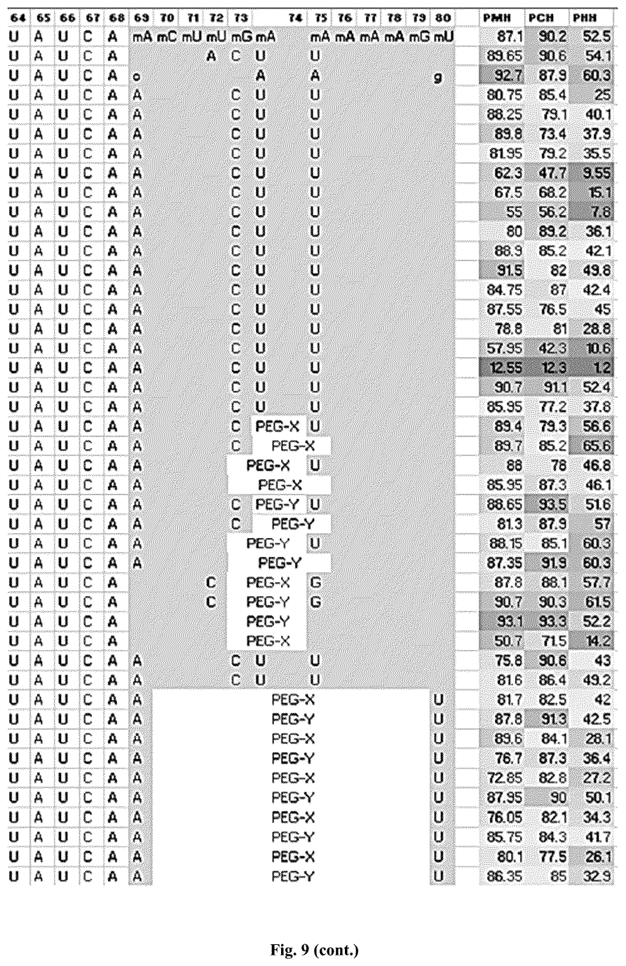

LNPs containing sgRNAs indicated in Table 15 were administered to female Sprague Dawley rats (n=5) at a dose of 0.1 mg/kg and 0.03 mg/kg of total RNA as described above. Guides G000534 and G018631 served as the control. Table 15 shows the editing efficiency, serum TTR protein, and percent TSS, respectively. Editing efficiency and serum TTR protein levels are illustrated in FIGS. 8A and 8B.

TABLE 15
Liver Editing and Serum TTR

| | LNP | | | | | |
|---|---|---|---|---|---|---|
| | Dose | Mean | | Mean serum | | |
| Guide | (mg/kg) | % indel | SD | TTR (ug/ml) | SD | % TSS |
| TSS | n/a | 0.1 | 0.0 | 1385.4 | 215.8 | 100.0 |
| G000534 | 0.03 | 4.8 | 2.4 | 1200.3 | 343.4 | 86.6 |
| | 0.1 | 40.4 | 5.7 | 596.3 | 91.7 | 43.0 |
| G018631 | 0.03 | 25.8 | 4.7 | 770.1 | 181.4 | 55.6 |
| | 0.1 | 62.4 | 2.7 | 200.0 | 88.9 | 14.4 |
| G022497 | 0.03 | 16.0 | 3.4 | 839.6 | 45.5 | 60.6 |
| | 0.1 | 46.3 | 8.1 | 521.8 | 137.4 | 37.7 |
| G022498 | 0.03 | 4.2 | 1.6 | 1052.6 | 207.4 | 76.0 |
| | 0.1 | 20.9 | 8.7 | 781.1 | 143.6 | 56.4 |
| G022499 | 0.03 | 8.6 | 6.2 | 978.5 | 111.8 | 70.6 |
| | 0.1 | 42.8 | 9.7 | 489.3 | 185.1 | 35.3 |
| G022500 | 0.03 | 2.6 | 1.1 | 905.0 | 146.7 | 65.3 |
| | 0.1 | 16.1 | 6.1 | 828.3 | 59.8 | 59.8 |

### Example 6. sgRNA:mRNA Ratio Relative to sgRNA or pgRNA Using LNPs

Studies were conducted to evaluate the editing efficiency of sgRNA designs that contain PEG linkers (pgRNA). The study compared two gRNAs targeting TTR with the same guide sequence, one of which included three PEG linkers in the constant region of the guide (pgRNA, G021846) and one of which did not (G021845) as shown in Table 4B. The guides and mRNA were formulated in separate LNPs and mixed to the desired ratios for delivery to primary mouse hepatocytes (PMH) via lipid nanoparticles (LNPs).

PMH cells were prepared, treated, and analyzed as described in Example 1 unless otherwise noted. PMH cells from In Vitro ADMET Laboratories (Lot #MCM114) were plated at a density of 15,000 cells/well. Cells were treated with LNPs as described below. LNPs were generally prepared as described in Example 1. LNPs were prepared with a lipid composition at a molar ratio of 50% Lipid A, 38% cholesterol, 9% DSPC, and 3% PEG2k-DMG. The LNPs were formulated with a lipid amine to RNA phosphate (N:P) molar ratio of about 6. LNPs encapsulated a single RNA species, either gRNA G021845, gRNA G021846 or mRNA (mRNA M) as described in Example 1.

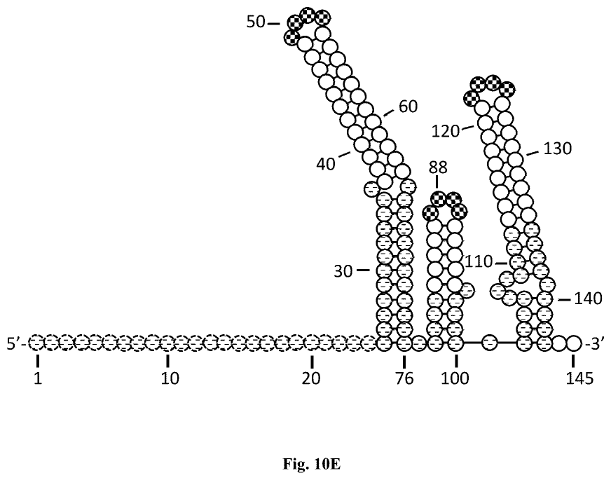

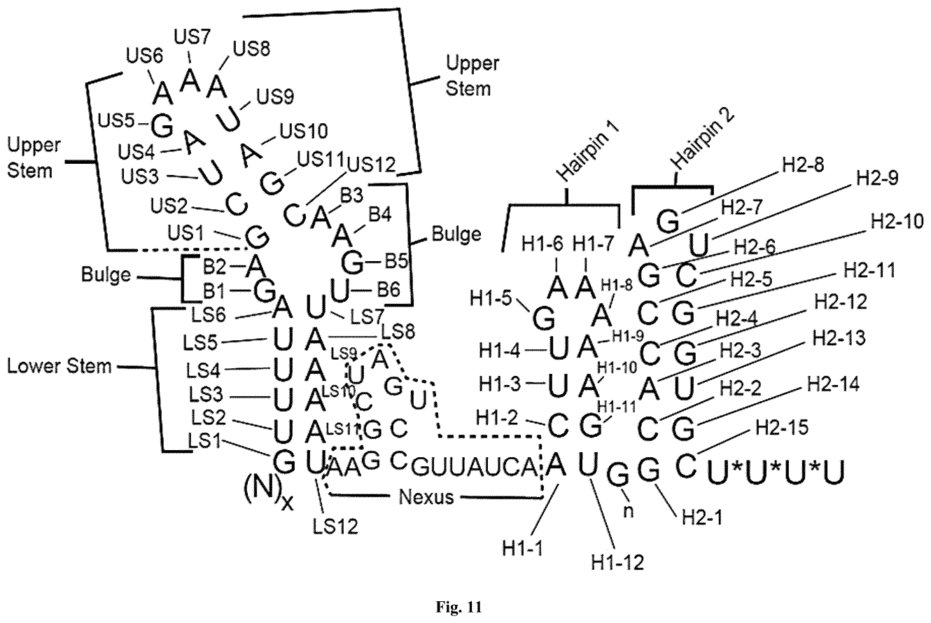

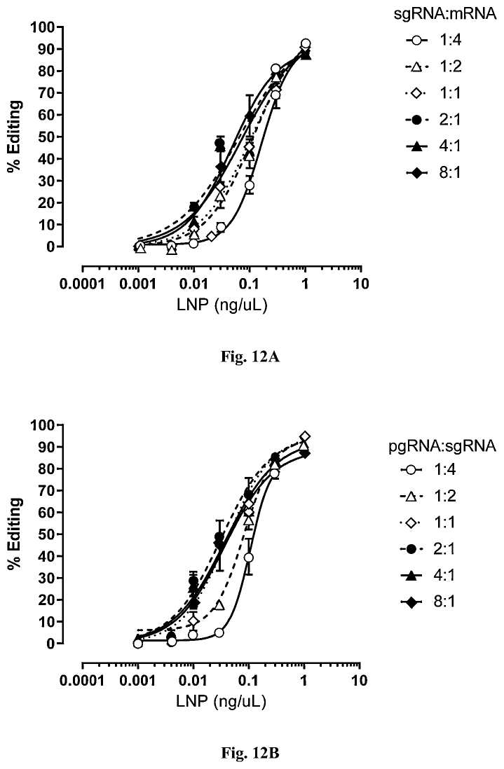

PMH cells were treated with varying amounts of LNPs at ratios of gRNA to mRNA of 1:4, 1:2, 1:1, 2:1, 4:1, or 8:1 by weight of RNA cargo. Duplicate samples were included in each assay. Guides were assayed in an 8 point 3-fold dose response curve starting at 1 ng/uL total RNA concentration as shown in Table 16. Mean percent editing results are shown in Table 16. FIG. 12A shows mean percent editing for sgRNA G021845 and FIG. 12B shows mean percent editing for sgRNA G021846. “ND” in the table represents values that could not be detected due to experimental failure.

TABLE 16
Mean percent editing in PMH

| | | sgRNA | | pgRNA | |
|---|---|---|---|---|---|
| | | (G021845) | | (G021846 | |
| Cargo ratio | LNP dose | Mean % | | Mean % | |
| (gRNA:mRNA) | (ng/uL) | editing | SD | editing | SD |
| 1:4 | 1 | 88.1 | 1.7 | ND | ND |
| | 0.3 | 68.7 | 5.7 | 78 | 0.3 |
| | 0.1 | 28.1 | 4.1 | 39.8 | 8.2 |
| | 0.03 | 8.7 | 2 | 5.1 | 0 |
| | 0.01 | 1.5 | 0.4 | 4 | 1.2 |
| | 0.004 | 0.6 | 0.5 | 0.2 | 0 |
| | 0.001 | 0.3 | 0.2 | 0.6 | 0.3 |
| 1:2 | 1 | 90.6 | 0 | 91.2 | 2.9 |
| | 0.3 | 78 | 2.4 | 85.6 | 1.4 |
| | 0.1 | 41.5 | 5.8 | 56.6 | 4.4 |
| | 0.03 | 23 | 5.4 | 17.5 | 0 |
| | 0.01 | 6.1 | 4.3 | 18.6 | 0.5 |
| | 0.004 | 0.1 | 0.1 | 3.4 | 1.7 |
| | 0.001 | 0.1 | 0 | 2.4 | 0.7 |
| 1:1 | 1 | 90.9 | 1.4 | 94.7 | 0.6 |
| | 0.3 | 71.8 | 4.2 | 84.7 | 0.9 |
| | 0.1 | 45.7 | 3.2 | 64.3 | 5.3 |
| | 0.03 | 27.4 | 1 | 44.8 | 11.5 |
| | 0.01 | 4.7 | 2.5 | 10.2 | 4.3 |
| | 0.004 | 0.2 | 0 | 1.7 | 0.7 |
| | 0.001 | 0.1 | 0 | 0.7 | 0.5 |
| 2:1 | 1 | 92.4 | 1.6 | 94.5 | 0.8 |
| | 0.3 | 80 | 1.3 | 85.7 | 0.2 |
| | 0.1 | 45.4 | 0 | 68 | 7.9 |
| | 0.03 | 47.2 | 3 | 49.3 | 0 |
| | 0.01 | 18.1 | 1.8 | 28.8 | 4.1 |
| | 0.004 | 0.8 | 0.7 | 3.8 | 2.4 |
| | 0.001 | 0.2 | 0.1 | 0.8 | 0.3 |
| 4:1 | 1 | 87.9 | 1.9 | 90.1 | 0 |
| | 0.3 | 80.2 | 2.2 | 84 | 0.1 |
| | 0.1 | 43.4 | 0 | 60.4 | 0.1 |
| | 0.03 | 46.2 | 0.5 | 46.1 | 0 |
| | 0.01 | 11.3 | 2.3 | 26.7 | 4.9 |
| | 0.004 | 0.4 | 0.2 | 1.5 | 0.4 |
| | 0.001 | 0.4 | 0.1 | 0.5 | 0.3 |
| 8:1 | 1 | 89.2 | 0 | 87.5 | 0 |
| | 0.3 | 76.7 | 3.9 | 78.6 | 3.1 |
| | 0.1 | 59.5 | 9.4 | 59.4 | 1.1 |
| | 0.03 | 36.4 | 7 | 45.3 | 0.5 |
| | 0.01 | 8.2 | 1.2 | 18.7 | 2.9 |
| | 0.004 | 0.6 | 0.6 | 2.6 | 0.3 |
| | 0.001 | 0.1 | 0 | 0.6 | 0.2 |

### Example 7. In Vitro Editing of Modified Pegylated Guides (pgRNAs) in PMH Using LNPs

Modified pgRNA having the same targeting site in the mouse TTR gene were assayed to evaluate the editing efficiency in PMH cells.

PMH cells were prepared, treated, and analyzed as described in Example 1 unless otherwise noted. PMH cells from In Vitro ADMET Laboratories (Lot #MC148) were used and plated at a density of 15,000 cells/well. LNP formulations were prepared as described in Example 1. LNPs were prepared with a lipid composition at a molar ratio of 50% Lipid A, 38% cholesterol, 9% DSPC, and 3% PEG2k-DMG. The LNPs were formulated with a lipid amine to RNA phosphate (N:P) molar ratio of about 6 and a gRNA as indicated in Table 17, or an mRNA.

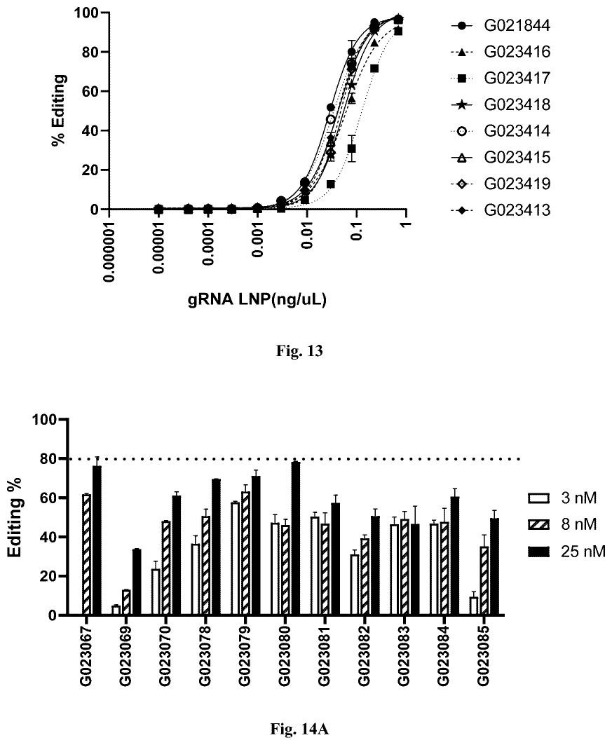

PMH in 100 ul media were treated with LNP for 30 ng total mRNA (mRNA P) by weight and LNP for gRNA in the amounts indicated in Table 17. Samples were run in duplicate. Mean editing results for PMH are shown in Table 17 and in FIG. 13.

TABLE 17
Mean percent editing in PMH

| | LNP | | | | LNP | | |
|---|---|---|---|---|---|---|---|
| | sgRNA | Mean % | | | sgRNA | Mean % | |
| Guide ID | (ng/uL) | editing | SD | Guide ID | (ng/uL) | editing | SD |
| G021844 | 0.7 | 96.6 | 0.5 | G023416 | 0.7 | 91.5 | 1.8 |
| | 0.23 | 95.0 | 0.5 | | 0.23 | 84.8 | 0.1 |
| | 0.08 | 80.0 | 5.9 | | 0.08 | 56.4 | 2.7 |
| | 0.03 | 51.9 | 1.9 | | 0.03 | 28.2 | 3.6 |
| | 0.009 | 13.9 | 0.4 | | 0.009 | 10.0 | 1.7 |
| | 0.003 | 4.6 | 0.9 | | 0.003 | 3.2 | 0.0 |
| | 0.001 | 0.8 | 0.1 | | 0.001 | 0.8 | 0.3 |
| | 0.0003 | 0.2 | 0.1 | | 0.0003 | 0.2 | 0.0 |
| | 0.0001 | 0.1 | 0.0 | | 0.0001 | 0.1 | 0.0 |
| | 0.00004 | 0.2 | 0.1 | | 0.00004 | 0.1 | 0.0 |
| | 0.00001 | 0.1 | 0.0 | | 0.00001 | 0.1 | 0.0 |
| | 0 | 0.1 | 0.0 | | 0 | 0.1 | 0.0 |
| G023413 | 0.7 | 96.4 | 0.4 | G023417 | 0.7 | 90.5 | 1.8 |
| | 0.23 | 92.5 | 0.7 | | 0.23 | 71.6 | 0.2 |
| | 0.08 | 73.9 | 0.9 | | 0.08 | 30.9 | 6.7 |
| | 0.03 | 36.4 | 2.6 | | 0.03 | 12.8 | 1.3 |
| | 0.009 | 10.3 | 1.5 | | 0.009 | 4.8 | 1.5 |
| | 0.003 | 2.4 | 0.7 | | 0.003 | 0.4 | 0.4 |
| | 0.001 | 0.6 | 0.1 | | 0.001 | 0.2 | 0.1 |
| | 0.0003 | 0.3 | 0.0 | | 0.0003 | 0.1 | 0.0 |
| | 0.0001 | 0.1 | 0.0 | | 0.0001 | 0.1 | 0.1 |
| | 0.00004 | 0.1 | 0.0 | | 0.00004 | 0.1 | 0.0 |
| | 0.00001 | 0.1 | 0.0 | | 0.00001 | 0.1 | 0.0 |
| | 0 | 0.1 | 0.0 | | 0 | 0.1 | 0.0 |
| G023414 | 0.7 | 96.5 | 0.2 | G023418 | 0.7 | 96.8 | 0.3 |
| | 0.23 | 92.7 | 0.4 | | 0.23 | 90.8 | 1.7 |
| | 0.08 | 74.1 | 2.7 | | 0.08 | 63.3 | 1.8 |
| | 0.03 | 45.7 | 1.5 | | 0.03 | 27.7 | 2.4 |
| | 0.009 | 13.7 | 0.7 | | 0.009 | 8.8 | 0.5 |
| | 0.003 | 4.3 | 1.3 | | 0.003 | 1.9 | 0.6 |
| | 0.001 | 0.7 | 0.1 | | 0.001 | 0.7 | 0.2 |
| | 0.0003 | 0.2 | 0.0 | | 0.0003 | 0.2 | 0.1 |
| | 0.0001 | 0.2 | 0.1 | | 0.0001 | 0.1 | 0.0 |
| | 0.00004 | 0.2 | 0.1 | | 0.00004 | 0.1 | 0.0 |
| | 0.00001 | 0.1 | 0.0 | | 0.00001 | 0.1 | 0.0 |
| | 0 | 0.1 | 0.0 | | 0 | 0.2 | 0.1 |
| G023415 | 0.7 | 96.5 | 0.5 | G023419 | 0.7 | 96.6 | 0.6 |
| | 0.23 | 92.6 | 0.7 | | 0.23 | 93.4 | 1.3 |
| | 0.08 | 73.1 | 0.2 | | 0.08 | 71.1 | 3.3 |
| | 0.03 | 34.4 | 0.8 | | 0.03 | 29.0 | 4.6 |
| | 0.009 | 14.2 | 0.2 | | 0.009 | 9.7 | 4.1 |
| | 0.003 | 3.9 | 0.4 | | 0.003 | 2.3 | 0.5 |
| | 0.001 | 0.5 | 0.2 | | 0.001 | 0.4 | 0.0 |
| | 0.0003 | 0.2 | 0.0 | | 0.0003 | 0.1 | 0.0 |
| | 0.0001 | 0.2 | 0.0 | | 0.0001 | 0.2 | 0.0 |
| | 0.00004 | 0.1 | 0.0 | | 0.00004 | 0.2 | 0.0 |
| | 0.00001 | 0.1 | 0.0 | | 0.00001 | 0.1 | 0.0 |
| | 0 | 0.1 | 0.0 | | 0 | 0.1 | 0.0 |

### Example 8. Evaluation of Guide Sequence Chemical Modifications in PMH

Pegylated guide RNA (pgRNA) with chemical modifications in the guide sequence were tested for editing efficiency at two distinct mouse TTR regions (Exon 1 and Exon 3) in PMH. PMH (In Vitro ADMET Laboratories) were prepared as described in Example 1 with a plating density of 20,000 cells/well. Lipofection of Nme2 Cas9 mRNA (mRNA 0; SEQ ID NO: 367) and gRNAs targeting two distinct loci in mouse TTR as indicated in Table 18 used pre-mixed lipid compositions as described in Example 1. Lipoplexes were used to treat cells with 100 ng/100 ul Nme2 mRNA and with gRNA at the concentrations indicated in Table 18. Cells were incubated in maintenance media +10% FBS (Corning #35-010-CF) at 37° C. for 72 hours. Post incubation, genomic DNA was isolated and NGS analysis was performed as described in Example 1.

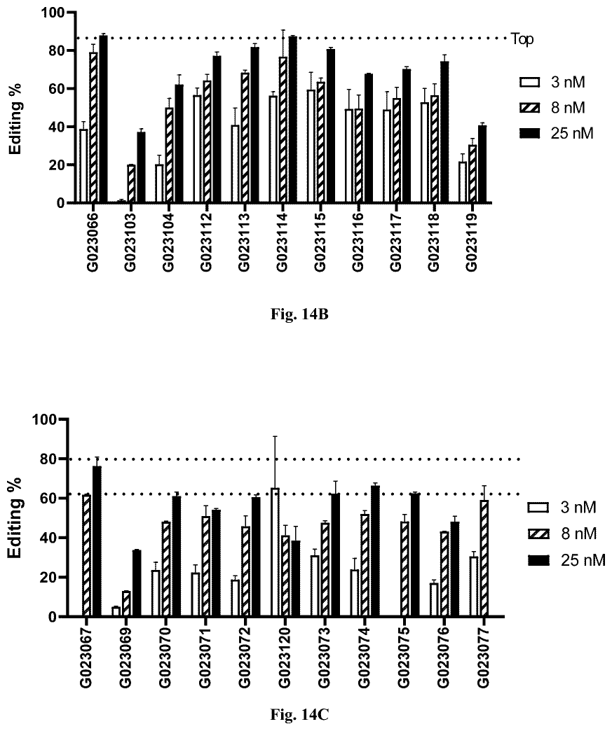

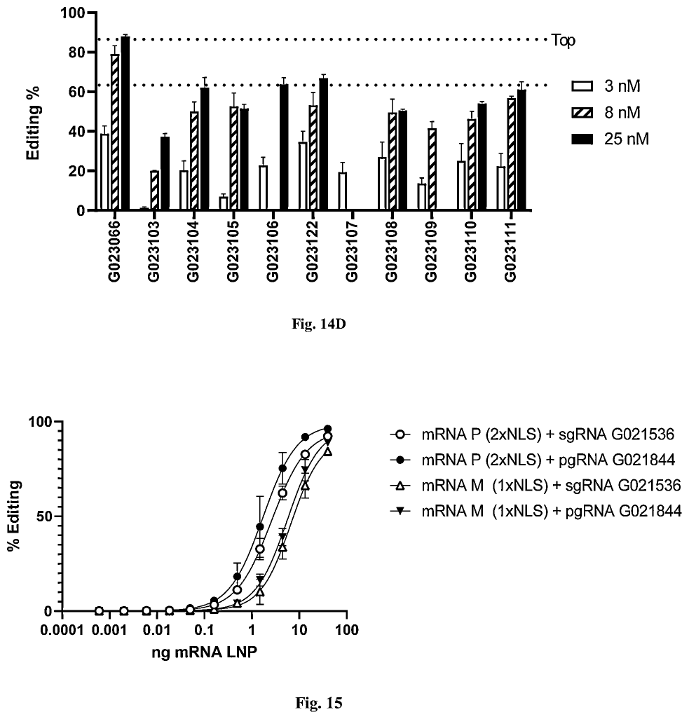

Editing efficiency was determined for various guide modification patterns at three gRNA concentrations (3 nM, 8 nM, or 25 nM). Duplicate samples were included in the assay. Mean editing results are shown in Table 18 and FIGS. 14A-14B for test guides with the N79 pgRNA design (G023066 or G023067) that are lacking a 2′-OMe at specified nucleotide positions in the target-binding region of the gRNA. Table 19 and FIGS. 14C-14D show mean percent editing for test guides with the End-Mod pgRNA designs (G023070 or G023104) with additional 2′-OMe modifications at the specified nucleotide position in the target-binding region of the gRNA. “ND” in the table represents values that could not be detected due to experimental failure.

TABLE 18
Mean percent editing for N79 pgRNAs lacking 2′-OMe modification
at the specified position in the guide sequence.

| | | | gRNA concentrations | | | | | |
|---|---|---|---|---|---|---|---|---|
| | | | 3 nM | | 8 nM | | 25 nM | |
| | Guide Sequence | | Mean | | Mean | | Mean | |
| Locus | Modification | Guide | % Edit | SD | % Edit | SD | % Edit | SD |
| Exon-1 | High mod pgRNA | G023067 | ND | ND | 61.8 | 0.3 | 76.3 | 4.6 |
| | No-Mod | G023069 | 5.1 | 0.4 | 13.0 | 0.2 | 33.8 | 0.3 |
| | End-Mod | G023070 | 23.7 | 3.9 | 48.1 | 0.3 | 61.2 | 2.0 |
| | POSITION 4 | G023078 | 36.5 | 4.1 | 50.7 | 3.5 | 69.5 | 0.1 |
| | POSITION 5 | G023079 | 57.7 | 0.5 | 63.2 | 3.5 | 71.2 | 3.0 |
| | POSITION 8 | G023080 | 47.3 | 4.2 | 46.1 | 2.9 | 78.4 | 0.2 |
| | POSITION 9 | G023081 | 50.4 | 2.2 | 46.8 | 5.6 | 57.4 | 4.1 |
| | POSITION 11 | G023082 | 31.2 | 2.3 | 39.3 | 1.9 | 50.7 | 3.6 |
| | POSITION 13 | G023083 | 46.5 | 3.7 | 49.2 | 3.8 | 46.6 | 9.2 |
| | POSITION 18 | G023084 | 46.8 | 1.8 | 47.7 | 7 | 60.7 | 4.1 |
| | POSITION 22 | G023085 | 9.5 | 2.6 | 35.2 | 5.8 | 49.6 | 3.9 |
| Exon-3 | High mod pgRNA | G023066 | 38.8 | 4.0 | 79.2 | 4.2 | 88.0 | 1.0 |
| | No-Mod | G023103 | 1.3 | 0.5 | 20 | 0.2 | 37.3 | 1.6 |
| | End-Mod | G023104 | 20.3 | 4.7 | 50.1 | 4.9 | 62.1 | 5.1 |
| | POSITION 4 | G023112 | 56.7 | 3.8 | 64.3 | 3.2 | 77.2 | 2.0 |
| | POSITION 5 | G023113 | 41.0 | 8.9 | 68.4 | 1.3 | 81.8 | 1.8 |
| | POSITION 8 | G023114 | 56.3 | 2.2 | 76.8 | 14 | 87.5 | 0.5 |
| | POSITION 9 | G023115 | 59.5 | 9.0 | 63.6 | 1.9 | 80.8 | 0.9 |
| | POSITION 11 | G023116 | 49.4 | 10.3 | 49.5 | 7.1 | 67.8 | 0.2 |
| | POSITION 13 | G023117 | 49.0 | 9.4 | 55.1 | 5.7 | 70.3 | 1.2 |
| | POSITION 18 | G023118 | 52.9 | 7.3 | 56.6 | 6.0 | 74.4 | 3.4 |
| | POSITION 22 | G023119 | 21.7 | 4.1 | 30.5 | 3.3 | 40.8 | 1.2 |

ND = no data reported due to technical failure.

TABLE 19
Mean percent editing for end modified pgRNAs with an additional 2′-OMe modification
at the specified position in the target-binding region of the pgRNAs.

| | | | gRNA concentrations | | | | | |
|---|---|---|---|---|---|---|---|---|
| | | | 3 nM | | 8 nM | | 25 nM | |
| | | | Mean | | Mean | | Mean | |
| | Guide Sequence | | % | | % | | % | |
| Locus | Modification | Guide | Edit | SD | Edit | SD | Edit | SD |
| Exon-1 | High mod pgRNA | G023067 | ND | ND | 61.8 | 0.3 | 76.3 | 4.6 |
| | No-Mod | G023069 | 5.1 | 0.4 | 13.0 | 0.2 | 33.8 | 0.3 |
| | End-Mod | G023070 | 23.7 | 3.9 | 48.1 | 0.3 | 61.2 | 2.0 |
| | POSITION 4 | G023071 | 22.4 | 4.0 | 51.0 | 5.3 | 54.2 | 0.6 |
| | POSITION 5 | G023072 | 18.8 | 2.1 | 45.8 | 5.3 | 60.5 | 1.2 |
| | POSITION 8 | G023120 | 65.3 | 26 | 41.3 | 5.0 | 38.6 | 7.2 |
| | POSITION 9 | G023073 | 31.1 | 3.1 | 47.7 | 1.0 | 62.4 | 6.3 |
| | POSITION 11 | G023074 | 24.0 | 5.6 | 52.0 | 1.7 | 66.5 | 1.4 |
| | POSITION 13 | G023075 | ND | ND | 48.2 | 3.6 | 62.5 | 0.8 |
| | POSITION 18 | G023076 | 17.2 | 1.6 | 43.1 | 0.2 | 48.1 | 2.8 |
| | POSITION 22 | G023077 | 30.6 | 2.5 | 59.1 | 7.2 | ND | ND |
| Exon-3 | High mod pgRNA | G023066 | 38.8 | 4.0 | 79.2 | 4.2 | 88.0 | 1.0 |
| | No-Mod | G023103 | 1.3 | 0.5 | 20.0 | 0.2 | 37.3 | 1.6 |
| | End-Mod | G023104 | 20.3 | 4.7 | 50.1 | 4.9 | 62.1 | 5.1 |
| | POSITION 4 | G023105 | 7.0 | 1.4 | 52.6 | 6.8 | 51.6 | 2.2 |
| | POSITION 5 | G023106 | 22.8 | 4.2 | ND | ND | 63.8 | 3.4 |
| | POSITION 8 | G023122 | 34.6 | 5.4 | 53.2 | 6.5 | 66.9 | 1.9 |
| | POSITION 9 | G023107 | 19.3 | 5.1 | ND | ND | ND | ND |
| | POSITION 11 | G023108 | 27.1 | 7.5 | 49.6 | 6.8 | 50.5 | 0.7 |
| | POSITION 13 | G023109 | 13.6 | 2.8 | 41.6 | 3.4 | ND | ND |
| | POSITION 18 | G023110 | 25.1 | 8.8 | 46.2 | 3.9 | 54.1 | 1.1 |
| | POSITION 22 | G023111 | 22.3 | 6.6 | 56.8 | 1.1 | 61.2 | 3.9 |

ND = no data reported due to technical failure.

### Example 9. Dose Response of Nme2 NLS Variants Using LNPs in PMH

Messenger mRNAs encoding Nme2Cas9 ORFs with different NLS placements were assayed for editing efficiency in primary mouse hepatocytes (PMH). The assay tested guides targeting the mouse TTR locus and included both sgRNA and pgRNA designs.

PMH were prepared as in Example 1 with a plating density of 20,000 cells/well. LNPs were generally prepared as described in Example 1 with a single RNA species as cargo, as indicated in Table 20. LNPs were prepared with the lipid composition at a molar ratio of 50% Lipid A, 38% cholesterol, 9% DSPC, and 3% PEG2k-DMG. The LNPs were formulated with a lipid amine to RNA phosphate (N:P) molar ratio of about 6.

Cells were treated with 60 ng/100 ul LNP containing gRNA by RNA weight and with LNP containing mRNA as indicated in Table 20. Cells were incubated for 72 hours at 37° C. in Williams' E Medium (Gibco, A1217601) with maintenance supplements and 10% fetal bovine serum. After 72 hours incubation at 37° C., cells were harvested and editing was assessed by NGS as described in Example 1. Mean percent editing data is shown in Table 20 and FIG. 15.

TABLE 20
Mean percent editing at the mouse TTR
locus in primary mouse hepatocytes.

| | mRNA LNP | Mean % | | |
|---|---|---|---|---|
| Sample | (ng RNA) | Editing | SD | N |
| mRNA P (2 × NLS) | 40.000 | 92.30 | 0.85 | 3 |
| G021536 | 13.330 | 82.67 | 1.61 | 3 |
| | 4.440 | 62.27 | 2.96 | 3 |
| | 1.480 | 32.80 | 4.54 | 3 |
| | 0.490 | 11.23 | 1.37 | 3 |
| | 0.160 | 3.40 | 0.71 | 3 |
| | 0.050 | 0.80 | 0.22 | 3 |
| | 0.018 | 0.30 | 0.08 | 3 |
| | 0.006 | 0.20 | 0.08 | 3 |
| | 0.002 | 0.13 | 0.05 | 3 |
| | 0.001 | 0.13 | 0.05 | 3 |
| | 0.000 | 0.10 | 0.00 | 3 |
| mRNA P (2 × NLS) | 40.000 | 96.17 | 0.12 | 3 |
| G021844 (pgRNA) | 13.330 | 91.83 | 0.34 | 3 |
| | 4.440 | 75.37 | 6.80 | 3 |
| | 1.480 | 44.53 | 13.11 | 3 |
| | 0.490 | 18.30 | 5.77 | 3 |
| | 0.160 | 5.50 | 1.43 | 3 |
| | 0.050 | 1.63 | 0.71 | 3 |
| | 0.018 | 0.33 | 0.05 | 3 |
| | 0.006 | 0.17 | 0.05 | 3 |
| | 0.002 | 0.07 | 0.05 | 3 |
| | 0.001 | 0.10 | 0.00 | 3 |
| | 0.000 | 0.07 | 0.05 | 3 |
| mRNA M (1 × NLS) | 40.000 | 84.27 | 1.23 | 3 |
| G021536 | 13.330 | 66.23 | 5.39 | 3 |
| | 4.440 | 33.80 | 5.14 | 3 |
| | 1.480 | 10.17 | 5.51 | 3 |
| | 0.490 | 4.20 | 0.92 | 3 |
| | 0.160 | 1.10 | 0.45 | 3 |
| | 0.050 | 0.33 | 0.17 | 3 |
| | 0.018 | 0.23 | 0.09 | 3 |
| | 0.006 | 0.10 | 0.00 | 3 |
| | 0.002 | 0.10 | 0.00 | 3 |
| | 0.001 | 0.10 | 0.00 | 3 |
| | 0.000 | 0.07 | 0.05 | 3 |
| mRNA M (1 × NLS) | 40.000 | 88.83 | 0.37 | 3 |
| G021844 (pgRNA) | 13.330 | 74.37 | 4.63 | 3 |
| | 4.440 | 39.00 | 3.72 | 3 |
| | 1.480 | 16.40 | 2.52 | 3 |
| | 0.490 | 4.03 | 0.77 | 3 |
| | 0.160 | 1.27 | 0.05 | 3 |
| | 0.050 | 0.23 | 0.05 | 3 |
| | 0.018 | 0.20 | 0.08 | 3 |
| | 0.006 | 0.10 | 0.00 | 3 |
| | 0.002 | 0.10 | 0.00 | 3 |
| | 0.001 | 0.10 | 0.00 | 3 |
| | 0.000 | 0.10 | 0.00 | 3 |

### Example 10. Dose Response of Nme2 NLS Variants Using LNPs in PMH

Messenger mRNAs encoding Nme2Cas9 ORFs with different NLS placements were assayed for editing efficiency in primary mouse hepatocytes (PMH).

PMH (Gibco, MC148) were prepared as described in Example 1 with a plating density of 20,000 cells/well. LNPs were generally prepared as described in Example 1 with a single RNA species as cargo. LNPs were prepared with the lipid composition at a molar ratio of 50% Lipid A, 38% cholesterol, 9% DSPC, and 3% PEG2k-DMG. The LNPs were formulated with a lipid amine to RNA phosphate (N:P) molar ratio of about 6.

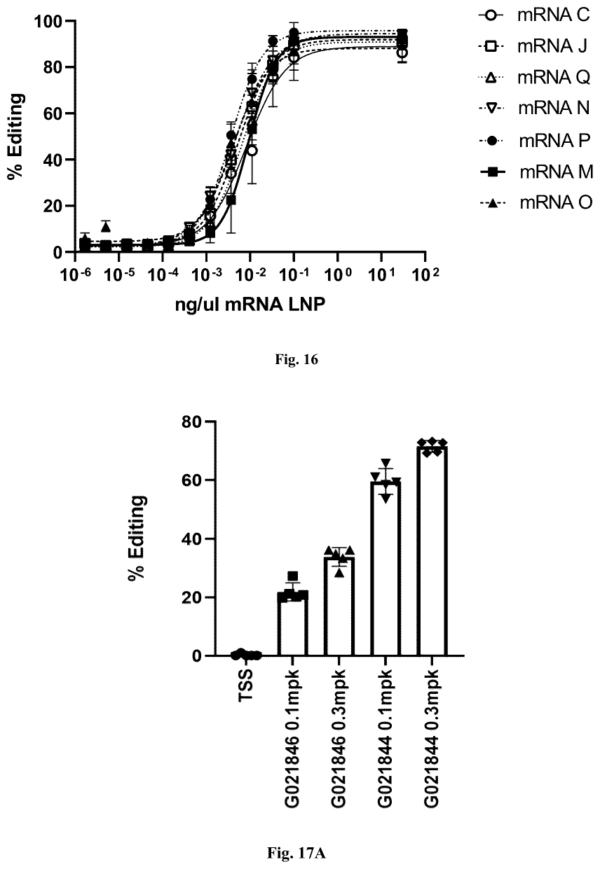

Cells were treated with 30 ng by RNA weight/100 ul of LNP containing gRNA G021844 and with LNP containing mRNA as indicated in Table 21. Cells were incubated at 37° C. for 24 hours in Williams' E Medium (Gibco, A1217601) with maintenance supplements and 10% fetal bovine serum. After 72 hours incubation at 37° C., cells were harvested and editing was assessed by NGS as described in Example 1. Mean percent editing data is shown in Table 21 and FIG. 16.

TABLE 21
Mean editing percentage in PMH treated with LNPs.

| | mRNA LNP | | | | EC₅₀ |
|---|---|---|---|---|---|
| mRNA | (ng/uL) | Mean | SD | N | (ng/uL) |
| mRNA C | 0.30 | 86.30 | 4.46 | 3 | 0.0082 |
| | 0.10 | 84.17 | 5.52 | | |
| | 0.03 | 75.80 | 1.91 | | |
| | 0.01 | 43.90 | 14.36 | | |
| | 0.004 | 34.03 | 8.64 | | |
| | 0.001 | 15.63 | 4.35 | | |
| | 0.0004 | 6.17 | 2.41 | | |
| | 0.0001 | 3.47 | 0.62 | | |
| | 0.00005 | 2.37 | 0.34 | | |
| | 0.00002 | 3.00 | 0.64 | | |
| | 0.00001 | 2.60 | 0.57 | | |
| | 0.00 | 2.70 | 0.16 | | |
| mRN J | 0.30 | 91.30 | 2.92 | 3 | 0.0053 |
| | 0.10 | 89.60 | 4.23 | | |
| | 0.03 | 80.93 | 8.17 | | |
| | 0.01 | 62.85 | 14.35 | | |
| | 0.004 | 39.95 | 5.15 | | |
| | 0.001 | 16.70 | 3.79 | | |
| | 0.0004 | 7.73 | 2.98 | | |
| | 0.0001 | 4.23 | 0.95 | | |
| | 0.00005 | 2.80 | 0.70 | | |
| | 0.00002 | 3.23 | 0.54 | | |
| | 0.00001 | 2.67 | 0.48 | | |
| | 0.00 | 3.60 | 0.57 | | |
| mRNA Q | 0.30 | 90.67 | 4.40 | 3 | 0.0065 |
| | 0.10 | 86.77 | 5.43 | | |
| | 0.03 | 80.27 | 6.65 | | |
| | 0.01 | 56.90 | 5.48 | | |
| | 0.004 | 35.45 | 1.35 | | |
| | 0.001 | 12.63 | 3.16 | | |
| | 0.0004 | 5.17 | 0.56 | | |
| | 0.0001 | 2.73 | 0.17 | | |
| | 0.00005 | 2.97 | 0.41 | | |
| | 0.00002 | 2.73 | 0.21 | | |
| | 0.00001 | 2.87 | 0.56 | | |
| | 0.00 | 2.43 | 0.82 | | |
| mRNA N | 0.30 | 93.93 | 2.20 | 3 | 00045 |
| | 0.10 | 90.97 | 1.77 | | |
| | 0.03 | 82.80 | 8.24 | | |
| | 0.01 | 68.67 | 10.18 | | |
| | 0.004 | 42.07 | 2.25 | | |
| | 0.001 | 24.13 | 4.21 | | |
| | 0.0004 | 10.60 | 0.94 | | |
| | 0.0001 | 4.67 | 0.66 | | |
| | 0.00005 | 3.30 | 1.84 | | |
| | 0.00002 | 3.37 | 0.69 | | |
| | 0.00001 | 2.53 | 0.90 | | |
| | 0.00 | 2.33 | 1.48 | | |
| mRNA P | 0.30 | 94.47 | 1.04 | 3 | 0.0036 |
| | 0.10 | 95.03 | 0.96 | | |
| | 0.03 | 91.27 | 2.36 | | |
| | 0.01 | 74.77 | 6.91 | | |
| | 0.004 | 50.57 | 4.89 | | |
| | 0.001 | 22.67 | 0.25 | | |
| | 0.0004 | 8.27 | 0.74 | | |
| | 0.0001 | 4.93 | 0.70 | | |
| | 0.00005 | 3.37 | 0.74 | | |
| | 0.00002 | 2.93 | 0.68 | | |
| | 0.00001 | 2.87 | 0.05 | | |
| | 0.00 | 2.87 | 0.45 | | |
| mRNA M | 0.30 | 92.00 | 0.80 | 3 | 0.0093 |
| | 0.10 | 91.40 | 1.90 | | |
| | 0.03 | 79.70 | 0.70 | | |
| | 0.01 | 53.10 | 6.80 | | |
| | 0.004 | 22.47 | 14.28 | | |
| | 0.001 | 8.20 | 4.20 | | |
| | 0.0004 | 4.57 | 1.57 | | |
| | 0.0001 | 2.73 | 0.31 | | |
| | 0.00005 | 3.07 | 0.21 | | |
| | 0.00002 | 2.93 | 0.09 | | |
| | 0.00001 | 2.77 | 0.66 | | |
| | 0.00 | 3.47 | 1.09 | | |
| mRNA O | 0.30 | 89.40 | 7.00 | 3 | 0.0042 |
| | 0.10 | 86.83 | 12.52 | | |
| | 0.03 | 78.17 | 15.41 | | |
| | 0.01 | 64.83 | 12.48 | | |
| | 0.004 | 47.33 | 9.03 | | |
| | 0.001 | 20.67 | 7.12 | | |
| | 0.0004 | 8.60 | 2.95 | | |
| | 0.0001 | 2.47 | 1.33 | | |
| | 0.00005 | 4.13 | 0.37 | | |
| | 0.00002 | 2.80 | 0.62 | | |
| | 0.00001 | 11.13 | 231. | | |
| | 0.00 | 6.13 | 2.16 | | |

### Example 11. In Vivo Editing Using pgRNA and mRNA LNPs

The editing efficiency of modified pgRNAs was evaluated in vivo. Four nucleotides in each of the loops of the repeat/anti-repeat region, hairpin 1, and hairpin 2 were substituted with Spacer-18 PEG linkers.

LNPs were generally prepared as described in Example 1 with a single RNA species as cargo. The LNPs contained a molar ratio of 50% Lipid A, 38% cholesterol, 9% DSPC, and 3% PEG2k-DMG. The LNPs were formulated with a lipid amine to RNA phosphate (N:P) molar ratio of about 6.

LNPs containing gRNAs targeting the TTR gene indicated in Table 22 were administered to female CD-1 mice (n=5) at a dose of 0.1 mg/kg or 0.3 mg/kg of total RNA as described above. LNP containing mRNA (mRNA M; SEQ ID NO: 365) and LNP containing a pgRNA (G021846 or G021844) were delivered simultaneously at a ratio of 1:2 by RNA weight, respectively. Mice were euthanized at 7 days post dose.

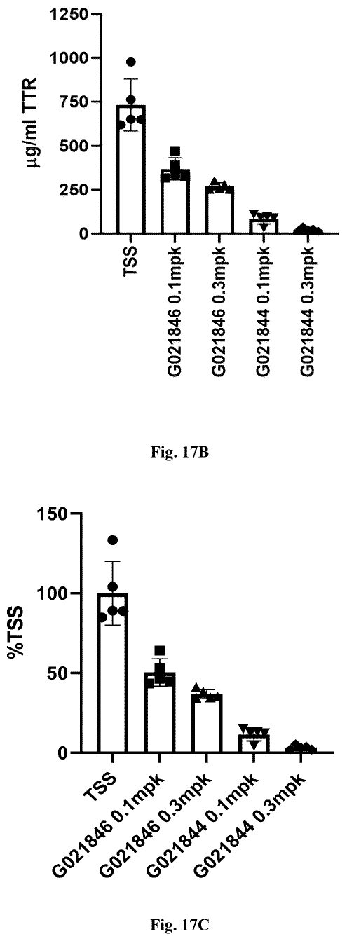

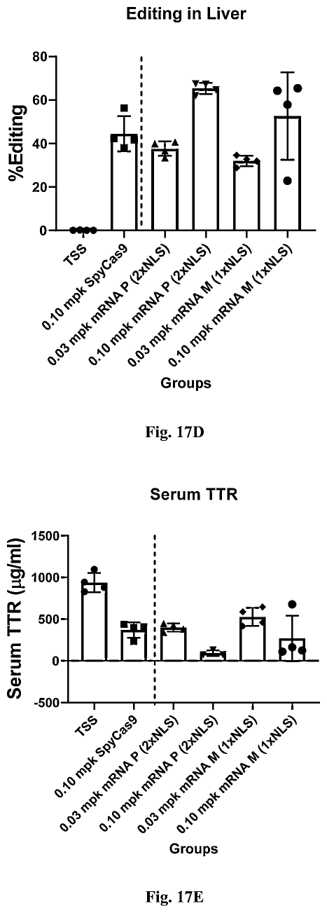

The editing efficiency, serum TTR knockdown, and percent TSS for the LNPs containing the indicated pgRNAs are shown in Table 22 and illustrated in FIGS. 17A-C respectively.

TABLE 22
Liver Editing, Serum TTR protein, and TTR protein knockdown

| | | | | Mean | | | |
|---|---|---|---|---|---|---|---|
| | | | | serum | | | |
| | Dose | Mean | | TTR | | Mean | |
| Guide | (mg/kg) | % Edit | SD | (ug/ml) | SD | % TSS | SD |
| TSS | NA | 0.1 | 0 | 733.1 | 131.2 | 100 | 17.9 |
| G021846 | 0.1 | 21.9 | 2.8 | 369.5 | 56.2 | 50.4 | 7.7 |
| | 0.3 | 33.8 | 2.9 | 269.8 | 21.3 | 36.8 | 2.6 |
| G021844 | 0.1 | 59.6 | 3.9 | 84.1 | 26.6 | 11.5 | 3.6 |
| | 0.3 | 71.6 | 1.8 | 24.4 | 9.2 | 3.3 | 1.2 |

A pgRNA (G021844) from the study described above was evaluated in mice with alternative mRNAs at varied dose levels. LNPs were generally prepared as described in Example 1 with a single RNA species as cargo. LNPs containing pgRNA (G21844) or mRNA (mRNA P or mRNA M) were formulated as described in Example 1. The LNPs used were prepared with a molar ratio of 50% Lipid A, 38% cholesterol, 9% DSPC, and 3% PEG2k-DMG. The LNPs were formulated with a lipid amine to RNA phosphate (N:P) molar ratio of about 6. Both G000502 and G021844 target exon 3 of the mouse TTR gene. LNP containing pgRNA and LNP containing mRNA were dosed simultaneously based on combined RNA weight at a ratio of 2:1 guide:mRNA by RNA weight, respectively. An additional LNP was co-formulated with G000502 and SpyCas9 mRNA at a ratio of 1:2 by weight, respectively, a preferred SpyCas9 guide:mRNA ratio.

LNPs with RNA cargo as indicated in Table 23 were administered to female CD-1 mice (n=4) at a dose of 0.1 mg/kg or 0.03 mg/kg of total RNA. The editing efficiency for LNPs containing the indicated gRNAs are shown in Table 23 and illustrated in FIGS. 17D-17E.

TABLE 23
Liver Editing and Serum TTR protein knockdown

| | | | | | Mean | |
|---|---|---|---|---|---|---|
| | | Dose | Mean | | serum TTR | |
| Guide | mRNA | (mg/kg) | % Edit | SD | (ug/ml) | SD |
| TSS | TSS | NA | 0.12 | 0.04 | 937.4 | 100.5 |
| G000502 | SpyCas9 | 0.1 | 44.50 | 6.9 | 370.7 | 80.1 |
| G021844 | mRNA P | 0.03 | 37.70 | 2.9 | 398.7 | 41.9 |
| | (SEQ ID | 0.1 | 65.40 | 2.2 | 92.8 | 27.5 |
| | NO: 368) | | | | | |
| | mRNA M | 0.03 | 32.02 | 2.1 | 527.4 | 93.6 |
| | (SEQ ID | 0.1 | 62.50 | 17.4 | 268.6 | 236.8 |
| | NO: 365) | | | | | |

### Example 12. In Vivo Editing with NmeCas9 and Either sgRNA or pgRNA

The editing efficiency of the modified pgRNAs tested with Nme2Cas9 was tested in a mouse model. All Nine sgRNAs tested comprised the same 24-nucleotide guide sequence targeting mTTR.

LNPs were generally prepared as described in Example 1 with a single RNA species as cargo. The LNPs used were prepared with a molar ratio of 50% Lipid A, 38% cholesterol, 9% DSPC, and 3% PEG2k-DMG. The LNPs were formulated with a lipid amine to RNA phosphate (N:P) molar ratio of about 6. The LNPs were mixed at a ratio of 2:1 by weight of gRNA to mRNA cargo. Dose is calculated based on the combined RNA weight of gRNA and mRNA. Transport and storage solution (TSS) used in LNP preparation was dosed in the experiment as a vehicle-only negative control.

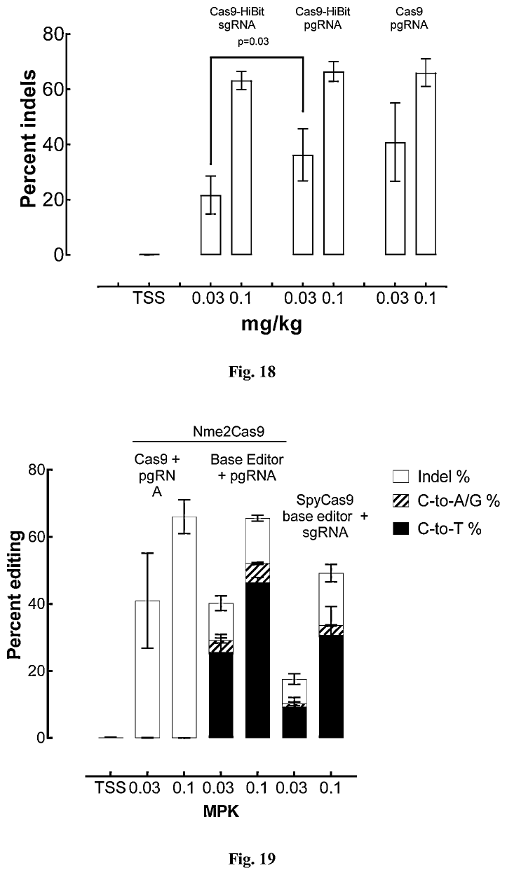

CD-1 female mice, ranging 6-10 weeks of age, were used in each study involving mice (n=5 per group, except TSS control n=4). Formulations were administered intravenously via tail vein injection according to the doses listed in Table 24. Animals were periodically observed for adverse effects for at least 24 hours post-dose. Six days after treatment, animals were euthanized by cardiac puncture under isoflurane anesthesia; liver tissue was collected for downstream analysis. Liver punches weighing between 5 and 15 mg were collected for isolation of genomic DNA and total RNA. Genomic DNA samples were analyzed with NGS sequencing as described in Example 1. The editing efficiency for LNPs containing the indicated mRNAs and gRNAs are shown in Table 24 and illustrated in FIG. 18.

TABLE 24
Mean percent editing in mouse liver

| | | Dose | Mean | | |
|---|---|---|---|---|---|
| mRNA | gRNA | (mg/kg) | % Edit | SD | N |
| TSS | TSS | — | 0.08 | 0.05 | 4 |
| mRNA P | G021536 | 0.03 | 21.68 | 6.87 | 5 |
| (2 × N term | (101-nt Nme | 0.1 | 63.22 | 3.28 | 5 |
| NLS, HiBit) | sgRNA) | | | | |
| mRNA P | G021844 | 0.03 | 36.28 | 9.45 | 5 |
| (2 × N term | (93-nt Nme | 0.1 | 66.44 | 3.55 | 5 |
| NLS, HiBit) | pgRNA) | | | | |
| mRNA O | G021844 | 0.03 | 40.88 | 14.16 | 5 |
| (2 × N-term | (93-nt Nme | 0.1 | 66.02 | 5.01 | 5 |
| NLS) | pgRNA) | | | | |

### Example 13. In Vivo Base Editing with gRNA

The editing efficiency of the modified gRNAs with different mRNAs were tested with Nine base editor construct in the mouse model. LNPs were generally prepared as described in Example 1 with a single RNA species as cargo. The LNPs used were prepared with a molar ratio of 50% Lipid A, 38% cholesterol, 9% DSPC, and 3% PEG2k-DMG. The LNPs were formulated with a lipid amine to RNA phosphate (N:P) molar ratio of about 6. The LNPs used were formulated as described in Example 1, except that each component, guide RNA, or mRNA was formulated individually into an LNP, and the LNP were mixed prior to administration as described in Table 25. For Nme2Cas9 and Nme2Cas9 base editor samples, LNPs were mixed at a ratio of 2:1 by weight of gRNA to editor mRNA cargo. For SpyCas9 base editor samples, LNPs were mixed at a ratio of 1:2 by weight of gRNA to editor mRNA cargo. Dose, as indicated in Table 31 and FIG. 14, is calculated based on the combined RNA weight of gRNA and editor mRNA. Base editor samples were treated with an additional 0.03 mg/kg of UGI mRNA. Transport and storage solution (TSS) used in LNP preparation was dosed in the experiment as a vehicle-only negative control.

CD-1 female mice, ranging 6-10 weeks of age, were used in each study involving mice (n=5 per group, except TSS control n=4). Formulations were administered intravenously via tail vein injection according to the doses listed in Table 25. Animals were periodically observed for adverse effects for at least 24 hours post-dose. Six days after treatment, animals were euthanized by cardiac puncture under isoflurane anesthesia; liver tissues were collected for downstream analysis. Liver punches weighing between 5 and 15 mg were collected for isolation of genomic DNA and total RNA. Genomic DNA was extracted using a DNA isolation kit (ZymoResearch, D3010) and samples were analyzed with NGS sequencing as described in Example 1. The editing efficiency for LNPs containing the indicated gRNAs are shown in Table 25 and illustrated in FIG. 19.

TABLE 25
Mean percent editing in mouse liver

| | Dose | C-to-T % | | | C-to-A/G % | | | Indel % | | |
|---|---|---|---|---|---|---|---|---|---|---|
| Sample | (mg/kg) | Mean | SD | n | Mean | SD | n | Mean | SD | n |
| TSS | 0 | 0.00 | 0.00 | 4 | 0.10 | 0.00 | 4 | 0.08 | 0.05 | 4 |
| mRNA O + G021844 | 0.03 | 0.00 | 0.00 | 5 | 0.08 | 0.04 | 5 | 40.88 | 14.16 | 5 |
| (Nme2Cas9 + pgRNA) | 0.1 | 0.00 | 0.00 | 5 | 0.02 | 0.04 | 5 | 66.02 | 5.01 | 5 |
| mRNA S + mRNA G + | 0.03 | 25.60 | 5.28 | 5 | 3.50 | 0.76 | 5 | 11.14 | 2.18 | 5 |
| G021844 | 0.1 | 46.34 | 1.53 | 5 | 5.74 | 0.33 | 5 | 13.52 | 0.90 | 5 |
| editor + UGI + | | | | | | | | | | |
| pgRNA) | | | | | | | | | | |
| mRNA E + mRNA G + | 0.03 | 9.28 | 2.82 | 5 | 0.94 | 0.54 | 5 | 7.34 | 1.61 | 5 |
| G000502 | 0.1 | 30.72 | 8.51 | 5 | 2.86 | 0.23 | 5 | 15.60 | 2.58 | 5 |
| (SpyBC22n + UGI + | | | | | | | | | | |
| sgRNA) | | | | | | | | | | |

### Example 14. In Vitro Editing in Human Hepatoma Cells Using Modified pgRNAs

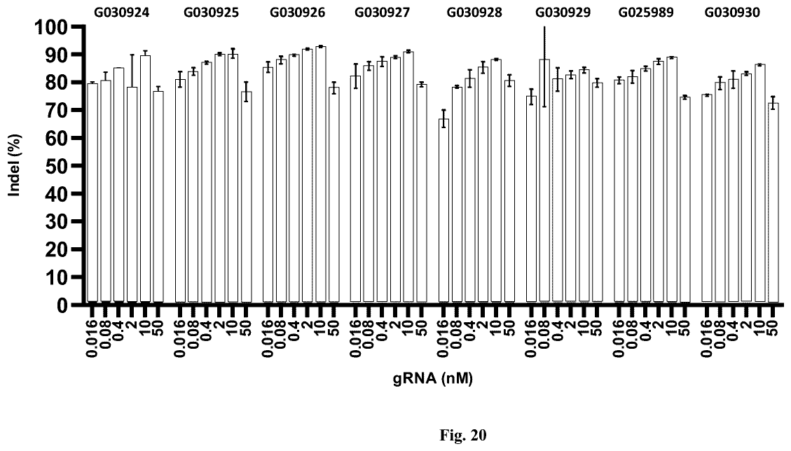

Guide RNAs targeting the same target sequence in the HEK3 genomic locus with various scaffold sequences were designed with truncations of the upper stem as shown in Table 2B. The gRNA were lipofected into human hepatoma (Huh7) cells to determine editing efficiency as follows. Cells were plated at a density of 15,000 cells/well. Lipofectamine™ MessengerMAX™ Reagent (Thermofisher) was used and samples were prepared according to the manufacturer's protocol with 50 ng of SpyCas9 mRNA (SEQ ID NO: 323)/reaction and an initial 50 nM guide concentration. Each guide RNA was serially diluted 5-fold for a 6-point dose response. Duplicate samples were included in the assay. Mean editing results with standard deviation (SD) are shown in Table 26 and FIG. 20.

TABLE 26
In vitro editing in Huh7 Cells

| | | gRNA (nM) | | | | | |
|---|---|---|---|---|---|---|---|
| Guide ID | | 0.016 | 0.08 | 0.4 | 2 | 10 | 50 |
| G030924 | Indel % | 79.40 | 80.45 | 84.95 | 78.00 | 89.35 | 76.65 |
| | SD | 0.71 | 3.18 | 0.21 | 11.88 | 1.91 | 1.77 |
| G030925 | Indel % | 81.00 | 83.75 | 86.95 | 90.00 | 90.20 | 76.50 |
| | SD | 2.83 | 1.48 | 0.49 | 0.42 | 1.70 | 3.54 |
| G030926 | Indel % | 85.30 | 87.90 | 89.70 | 91.85 | 92.80 | 77.80 |
| | SD | 1.98 | 1.27 | 0.28 | 0.21 | 0.14 | 2.12 |
| G030927 | Indel % | 82.05 | 85.80 | 87.40 | 88.90 | 90.90 | 79.00 |
| | SD | 4.45 | 1.56 | 1.70 | 0.57 | 0.57 | 0.99 |
| G030928 | Indel % | 66.70 | 78.15 | 81.25 | 85.35 | 88.15 | 80.45 |
| | SD | 3.25 | 0.64 | 3.18 | 2.05 | 0.35 | 2.19 |
| G030929 | Indel % | 74.60 | 88.00 | 80.85 | 82.55 | 84.25 | 79.65 |
| | SD | 2.83 | 16.97 | 4.31 | 1.48 | 1.06 | 1.63 |
| G025989 | Indel % | 80.60 | 81.90 | 84.90 | 87.35 | 88.90 | 74.40 |
| | SD | 1.27 | 2.26 | 0.85 | 1.06 | 0.14 | 0.71 |
| G030930 | Indel % | 75.25 | 79.60 | 80.90 | 83.05 | 86.15 | 72.45 |
| | SD | 0.21 | 2.26 | 3.11 | 0.64 | 0.21 | 2.33 |

### Example 15. Additional Embodiments

The following numbered items provide additional support for and descriptions of the embodiments herein.
Item 1 is a guide RNA (gRNA) comprising an internal linker.
Item 2 is the gRNA of item 1, wherein the internal linker substitutes for at least 2, 3, 4, 5, 6, 7, 8, 9, 10, 11, 12, 13, 14, 15, 16, 17, 18, 19, 20, 21, 22, 23, 24, 25, 26, 27, or 28 nucleotides of the gRNA.
Item 3 is the gRNA of item 1 or 2 wherein the internal linker has a bridging length of about 3-30, optionally 12-21 atoms, and the linker substitutes for at least 2 nucleotides of the gRNA.
Item 4 is the gRNA of any one of items 1-3, wherein the internal linker has a bridging length of about 6-18 atoms, optionally about 6-12 atoms, and the linker substitutes for at least 2 nucleotides of the gRNA.
Item 5 is the gRNA of any one of items 1-4, wherein the internal linker substitutes for 2-12 nucleotides.
Item 6 is the gRNA of any one of items 1-5, wherein the internal linker is in a repeat-anti-repeat region of the gRNA.
Item 7 is the gRNA of any one of items 1-6, wherein the internal linker substitutes for at least 4 nucleotides of the repeat-anti-repeat region of the gRNA.
Item 8 is the gRNA of any one of items 1-7, wherein the internal linker substitutes for up to 28 nucleotides of the repeat-anti-repeat region of the gRNA.
Item 9 is the gRNA of any one of items 1-8, wherein the internal linker substitutes for 2, 3, 4, 5, 6, 7, 8, 9, 10, 11, or 12 nucleotides of the repeat-anti-repeat region of the gRNA.
Item 10 is the gRNA of any one of items 1-9, wherein the internal linker is in a hairpin region of the gRNA.
Item 11 is the gRNA of any one of items 1-10, wherein the internal linker substitutes for at least 2 nucleotides of the hairpin region of the gRNA.
Item 12 is the gRNA of any one of items 1-11, wherein the internal linker substitutes for up to 22 nucleotides of the hairpin region of the gRNA.
Item 13 is the gRNA of any one of items 1-12, wherein the internal linker substitutes for 2, 3, 4, 5, 6, 7, 8, 9, 10, 11, 12, 13, 14, 15, 16, 17, 18, 19, 20, 21, or 22 nucleotides of the hairpin region of the gRNA.
Item 14 is the gRNA of any one of items 1-13, wherein the internal linker substitutes for 1, 2, 3, 4, 5, 6, 7, 8, or 9 base pairs of the hairpin region of the gRNA.
Item 15 is the gRNA of any one of items 1-14, wherein the internal linker is in a nexus region of the gRNA.
Item 16 is the gRNA of any one of items 1-15, wherein the internal linker substitutes for 1 or 2 nucleotides of the nexus region of the gRNA.
Item 17 is the gRNA of any one of items 1-16, wherein the internal linker is in a hairpin between a first portion of the gRNA and a second portion of the gRNA, wherein the first portion and the second portion together form a duplex portion.
Item 18 is the gRNA of any one of items 1-17, wherein the internal linker bridges a first portion of a duplex and a second portion of a duplex, wherein the duplex comprises 2-10 base pairs.
Item 19 is the gRNA of any one of items 1-18, wherein the gRNA comprises two internal linkers.
Item 20 is the gRNA of any one of items 1-18, wherein the gRNA comprises three internal linkers.
Item 21 is the gRNA of any one of items 1-20, wherein the internal linker in the repeat-anti-repeat region is in a hairpin between a first portion and a second portion of the repeat-anti-repeat region, wherein the first portion and the second portion together form a duplex portion.
Item 22 is the gRNA of item 21, wherein the internal linker in the repeat-anti-repeat region substitutes for 2, 3, 4, 5, 6, 7, 8, 9, 10, 11, 12, 13, 14, 15, 16, 17, 18, 19, 20, 21, 22, 23, 24, 25, 26, 27, or 28 nucleotides of the hairpin.
Item 23 is the gRNA of any one of items 21-22, wherein the internal linker in the repeat-anti-repeat region substitutes for at least 4 nucleotides of the hairpin.
Item 24 is the gRNA of any one of items 21-23, wherein the internal linker in the repeat-anti-repeat region substitutes for up to 28 nucleotides of the hairpin.
Item 25 is the gRNA of any one of items 21-24, wherein the internal linker in the repeat-anti-repeat region substitutes for 4-20 nucleotides of the hairpin.
Item 26 is the gRNA of any one of items 21-25, wherein the internal linker in the repeat-anti-repeat region substitutes for 4-14 nucleotides of the hairpin.
Item 27 is the gRNA of any one of items 21-26, wherein the internal linker in the repeat-anti-repeat region substitutes for 4-6 nucleotides of the hairpin.
Item 28 is the gRNA of any one of items 21-27, wherein the internal linker in the repeat-anti-repeat region substitutes for a loop, or part thereof, of the hairpin.
Item 29 is the gRNA of any one of items 21-28, wherein the internal linker in the repeat-anti-repeat region substitutes for the loop and the stem, or part thereof, of the hairpin.
Item 30 is the gRNA of any one of items 21-27, wherein the internal linker in the repeat-anti-repeat region substitutes for 2, 3, or 4 nucleotides of the loop of the hairpin.
Item 31 is the gRNA of any one of items 21-27, wherein the internal linker in the repeat-anti-repeat region substitutes for the loop of the hairpin and at least 1 nucleotide of the stem of the hairpin.
Item 32 is the gRNA of any one of items 21-31, wherein the internal linker in the repeat-anti-repeat region substitutes for the loop of the hairpin and 1, 2, 3, 4, 5, 6, 7, 8, 9, 10, 11, 12, 13, 14, 15, 16, 17, 18, 19, 20, 21, 22, 23, or 24 nucleotides of the stem of the hairpin.
Item 33 is the gRNA of any one of items 21-32, wherein the internal linker in the repeat-anti-repeat region substitutes for the loop of the hairpin and at least 2 nucleotides of the stem of the hairpin.
Item 34 is the gRNA of any one of items 21-32, wherein the internal linker in the repeat-anti-repeat region substitutes for the loop of the hairpin and 1, 2, 3, 4, 5, 6, 7, or 8 nucleotides of the stem of the hairpin.
Item 35 is the gRNA of any one of items 21-32, wherein the internal linker in the repeat-anti-repeat region substitutes for the loop of the hairpin and 1, 2, 3, 4, 5, 6, 7, 8, 9, 10, 11, or 12 base pairs of the stem of the hairpin.
Item 36 is the gRNA of any one of items 21-32, wherein the internal linker in the repeat-anti-repeat region substitutes for all of the nucleotides constituting the loop of the hairpin.
Item 37 is the gRNA of any one of items 21-32, wherein the internal linker in the repeat-anti-repeat region substitutes for all of the nucleotides constituting the loop and the stem of the hairpin.
Item 38 is the gRNA of any one of items 1-37, wherein the internal linker substitutes for 1 or 2 nucleotides of the nexus region of the gRNA.
Item 39 is the gRNA of any one of items 1-38, wherein the internal linker substitutes for a hairpin of the gRNA.
Item 40 is the gRNA of item 39, wherein the internal linker substitutes for 2, 3, 4, 5, 6, 7, 8, 9, 10, 11, 12, 13, 14, 15, 16, 17, 18, 19, 20, 21, or 22 nucleotides of the hairpin.
Item 41 is the gRNA of any one of items 39-40, wherein the internal linker substitutes for 2-22 nucleotides of the hairpin.
Item 42 is the gRNA of any one of items 39-41, wherein the internal linker substitutes for 2-12 nucleotides of the hairpin.
Item 43 is the gRNA of any one of items 39-42, wherein the internal linker substitutes for 2-6 nucleotides of the hairpin.
Item 44 is the gRNA of any one of items 39-43, wherein the internal linker substitutes for 2-4 nucleotides of the hairpin.
Item 45 is the gRNA of any one of items 39-44, wherein the internal linker substitutes for a loop, or part thereof, of the hairpin.
Item 46 is the gRNA of any one of items 39-45, wherein the internal linker substitutes for the loop and the stem, or part thereof, of the hairpin.
Item 47 is the gRNA of any one of items 39-46, wherein the internal linker substitutes for 2, 3, 4, or 5 nucleotides of the loop of the hairpin.
Item 48 is the gRNA of any one of items 39-47, wherein the internal linker substitutes for the loop of the hairpin and at least 1 nucleotide of the stem of the hairpin.
Item 49 is the gRNA of any one of items 39-48, wherein the internal linker substitutes for the loop of the hairpin and 1, 2, 3, 4, 5, 6, 7, 8, 9, 10, 11, 12, 13, 14, 15, 16, 17, or 18 nucleotides of the stem of the hairpin.
Item 50 is the gRNA of any one of items 39-49, wherein the internal linker substitutes for the loop of the hairpin and at least 2 nucleotides of the stem of the hairpin.
Item 51 is the gRNA of any one of items 39-50, wherein the internal linker in the repeat-anti-repeat region substitutes for the loop of the hairpin and up to 18 nucleotides of the stem of the hairpin.
Item 52 is the gRNA of any one of items 39-51, wherein the internal linker in the repeat-anti-repeat region substitutes for the loop of the hairpin and 1, 2, 3, 4, 5, 6, 7, 8, or 9 base pairs of the stem of the hairpin.
Item 53 is the gRNA of any one of items 39-52, wherein the internal linker substitutes for all of the nucleotides constituting the loop of the hairpin.
Item 54 is the gRNA of any one of items 39-53, wherein the internal linker substitutes for all of the nucleotides constituting the loop and the stem of the hairpin.
Item 55 is the gRNA of any one of items 39-54, wherein the hairpin is a hairpin 1.
Item 56 is the gRNA of any one of items 39-54, wherein the hairpin is a hairpin 2.
Item 57 is the gRNA of any one of items 39-54, wherein the hairpin is a hairpin 1, and the internal linker substitutes for the hairpin 1.
Item 58 is the gRNA of item 57, wherein the gRNA further comprises a hairpin 2 at 3′ to the hairpin 1.
Item 59 is the gRNA of item 58, wherein the internal linker substitutes for at least 2 nucleotides of a loop of the hairpin 2.
Item 60 is the gRNA of item 58 or 59, wherein the internal linker does not substitute for the hairpin 2.
Item 61 is the gRNA of any one of items 1-60, further comprising a guide region.
Item 62 is the gRNA of item 61, wherein the guide region is 17, 18, 19, or 20 nucleotides in length.
Item 63 is the gRNA of any one of items 1-62, wherein the gRNA is a single guide RNA (sgRNA).
Item 64 is the gRNA of any one of items 1-62, wherein the gRNA comprises a tracrRNA (trRNA).
Item 65 is a guide RNA (gRNA), wherein the gRNA is a single-guide RNA (sgRNA) comprising a guide region and a conserved portion at 3′ to the guide region, wherein the conserved portion comprises a repeat-anti-repeat region, a nexus region, a hairpin 1 region, and a hairpin 2 region, and comprises at least one of:
1) a first internal linker substituting for at least 2 nucleotides of an upper stem region of the repeat-anti-repeat region;
2) a second internal linker substituting for 1 or 2 nucleotides of the nexus region; and
3) a third internal linker substituting for at least 2 nucleotides of the hairpin 1.
Item 66 is the gRNA of item 65, wherein the sgRNA comprises the first internal linker and the second internal linker.
Item 67 is the gRNA of item 65, wherein the sgRNA comprises the first internal linker and the third internal linker.
Item 68 is the gRNA of item 65, wherein the sgRNA comprises the second internal linker and the third internal linker.
Item 69 is the gRNA of item 65, wherein the sgRNA comprises the first internal linker, the second internal linker, and the third internal linker.
Item 70 is the gRNA of any one of items 65-69, wherein the first internal linker has a bridging length of about 9-30 atoms, optionally about 15-21 atoms.
Item 71 is the gRNA of any one of items 65-70, wherein the first internal linker substitutes for 2, 3, 4, 5, 6, 7, 8, 9, 10, 11, or 12 nucleotides of the upper stem region.
Item 72 is the gRNA of any one of items 65-71, wherein the first internal linker substitutes for a loop, or part thereof, of the upper stem region.
Item 73 is the gRNA of any one of items 65-72, wherein the first internal linker substitutes for the loop and the stem, or part thereof, of the upper stem region.
Item 74 is the gRNA of any one of items 65-73, wherein the first internal linker substitutes for 2, 3, or 4 nucleotides of the loop of the upper stem region.
Item 75 is the gRNA of any one of items 65-74, wherein the first internal linker substitutes for the loop of the upper stem region and at least 2, 3, 4, 5, 6, 7, or 8 nucleotides of the stem of the upper stem region.
Item 76 is the gRNA of any one of items 65-75, wherein the first internal linker substitutes for the loop of the upper stem region and 1, 2, 3, or 4 base pairs of the stem of the upper stem region.
Item 77 is the gRNA of any one of items 65-76, wherein the first internal linker substitutes for all of the nucleotides constituting the loop of the upper stem region.
Item 78 is the gRNA of any one of items 65-77, wherein the first internal linker substitutes for all of the nucleotides constituting the loop and the stem of the upper stem region.
Item 79 is the gRNA of any one of items 65-78, wherein the second internal linker has a bridging length of about 6-18 atoms, optionally about 6-12 atoms.
Item 80 is the gRNA of any one of items 65-79, wherein the second internal linker substitutes for 2 nucleotides of the nexus region of the sgRNA.
Item 81 is the gRNA of any one of items 65-80, wherein the second internal linker substitutes for 2 nucleotides of a loop of the nexus region of the sgRNA.
Item 82 is the gRNA of any one of items 65-81, wherein the third internal linker has a bridging length of about 9-30, optionally about 12-21 atoms.
Item 83 is the gRNA of any one of items 65-82, wherein the third internal linker substitutes for 2, 3, 4, 5, 6, 7, 8, 9, 10, 11 or 12 nucleotides of the hairpin 1 of the gRNA.
Item 84 is the gRNA of any one of items 65-83, wherein the third linker substitutes for 1, 2, 3, 4, or 5 base pairs of the hairpin 1 of the gRNA.
Item 85 is the gRNA of any one of items 65-84, wherein the third internal linker substitutes for a loop, or part thereof, of the hairpin 1.
Item 86 is the gRNA of any one of items 65-85, wherein the third internal linker substitutes for the loop and the stem, or part thereof, of the hairpin 1.
Item 87 is the gRNA of any one of items 65-86, wherein the third internal linker substitutes for 2, 3, or 4 nucleotides of the loop of the hairpin 1.
Item 88 is the gRNA of any one of items 65-87, wherein the third internal linker substitutes for the loop of the hairpin and at least 1 nucleotide of the stem of the hairpin 1.
Item 89 is the gRNA of any one of items 65-88, wherein the third internal linker substitutes for the loop of the hairpin and 2, 4, or 6 nucleotides of the stem of the hairpin 1.
Item 90 is the gRNA of any one of items 65-89, wherein the third internal linker in the repeat-anti-repeat region substitutes for the loop of the hairpin and 1, 2, or 3 base pairs of the stem of the hairpin 1.
Item 91 is the gRNA of any one of items 65-90, wherein the third internal linker substitutes for all of the nucleotides constituting the loop of the hairpin 1.
Item 92 is the gRNA of any one of items 65-91, wherein the third internal linker substitutes for all of the nucleotides constituting the loop and the stem of the hairpin 1.
Item 93 is the gRNA of any one of items 65-92, wherein the hairpin 2 region of the sgRNA does not contain any internal linker.
Item 94 is the gRNA of any one of items 65-93, wherein the sgRNA is an S. pyogenes Cas9 sgRNA.
Item 95 is the gRNA of any one of items 65-94, wherein the sgRNA comprises a conserved portion comprising a sequence of SEQ ID NO: 400.
Item 96 is the gRNA of item 95, wherein 2, 3 or 4 of nucleotides 13-16 (US5-US8 of the upper stem region) are substituted for the first internal linker relative to SEQ ID NO: 400.
Item 97 is the gRNA of any one of items 95-96, wherein nucleotides 12-17 (US4-US9 of the upper stem region) are substituted for the first internal linker relative to SEQ ID NO: 400.
Item 98 is the gRNA of any one of items 95-97, wherein d nucleotides to 11-18 (US3-US10 of the upper stem region) are substituted for the first internal linker relative to SEQ ID NO: 400.
Item 99 is the gRNA of any one of items 95-98, wherein nucleotides to 10-19 (US2-US11 of the upper stem region) are substituted for the first internal linker relative to SEQ ID NO: 400.
Item 100 is the gRNA of any one of items 95-99, wherein nucleotides to 9-20 (US1-US10 of the upper stem region) are substituted for the first internal linker relative to SEQ ID NO: 400.
Item 101 is the gRNA of any one of items 95-100, wherein nucleotide 36-37 (N6-N7 of the nexus region) are substituted for the second internal linker relative to SEQ ID NO: 400.
Item 102 is the gRNA of any one of items 95-101, wherein 2, 3, or 4 of nucleotides 53-56 (H1-5-H1-8 of the hairpin 1) are substituted for the third internal linker relative to SEQ ID NO: 400.
Item 103 is the gRNA of any one of items 95-102, wherein nucleotides 52-57 (H1-4-H1-9 of the hairpin 1) are substituted for the third internal linker relative to SEQ ID NO: 400.
Item 104 is the gRNA of any one of items 95-103, wherein nucleotides 51-58 (H1-3-H1-10 of the hairpin 1) are substituted for the third internal linker relative to SEQ ID NO: 400.
Item 105 is the gRNA of any one of items 95-104, wherein nucleotides 50-59 (H1-1-H1-12 of the hairpin 1) are substituted for the third internal linker relative to SEQ ID NO: 400.
Item 106 is the gRNA of any one of items 95-105, wherein nucleotides 77-80 are deleted relative to SEQ ID NO: 400.
Item 107 is the gRNA of any one of items 65-94, wherein the sgRNA comprises a sequence of SEQ ID NO: 201.
Item 108 is the gRNA of item 107, wherein 2, 3 or 4 of nucleotides 33-36 are substituted for the first internal linker relative to SEQ ID NO: 201.
Item 109 is the gRNA of any one of items 107-108, wherein nucleotides 32-37 are substituted for the first internal linker relative to SEQ ID NO: 201.
Item 110 is the gRNA of any one of items 107-109, wherein nucleotides 31-38 are substituted for the first internal linker relative to SEQ ID NO: 201.
Item 111 is the gRNA of any one of items 107-110, wherein nucleotides 30-39 are substituted for the first internal linker relative to SEQ ID NO: 201.
Item 112 is the gRNA of any one of items 107-111, wherein nucleotides 29-40 are substituted for the first internal linker relative to SEQ ID NO: 201.
Item 113 is the gRNA of any one of items 107-112, wherein nucleotide 55-56 are substituted for the second internal linker relative SEQ ID NO: 201.
Item 114 is the gRNA of any one of items 107-113, wherein 2, 3, or 4 of nucleotides 50-53 are substituted for the third internal linker relative to SEQ ID NO: 201.
Item 115 is the gRNA of any one of items 107-114, wherein nucleotides 49-54 are substituted for the third internal linker relative to SEQ ID NO: 201.
Item 116 is the gRNA of any one of items 107-115, wherein nucleotides 77-80 are deleted relative to SEQ ID NO: 201.
Item 117 is a guide RNA (gRNA), wherein the gRNA is a single-guide RNA (sgRNA) comprising a guide region and a conserved portion at the 3′ to the guide region, wherein conserved portion comprises a repeat-anti-repeat region, a hairpin 1 region, and a hairpin 2 region, and further comprises at least one of:
1) a first internal linker substituting for at least 2 nucleotides of an upper stem region of the repeat-anti-repeat region of the sgRNA;
2) a second internal linker substituting for 1 or 2 nucleotides of the hairpin 1 of the sgRNA; or
3) a third internal linker substituting for at least 2 nucleotides of the hairpin 2 of the sgRNA.
Item 118 is the gRNA of item 117, wherein the sgRNA comprises the first internal linker and the second internal linker.
Item 119 is the gRNA of item 117, wherein the sgRNA comprises the first internal linker and the third internal linker.
Item 120 is the gRNA of item 117, wherein the sgRNA comprises the second internal linker and the third internal linker.
Item 121 is the gRNA of item 117, wherein the sgRNA comprises the first internal linker, the second internal linker, and the third internal linker.
Item 122 is the gRNA of any one of items 117-121, wherein the first internal linker has a bridging length of about 9-30 atoms, optionally about 15-21 atoms.
Item 123 is the gRNA of any one of items 117-122, wherein the first internal linker is in a hairpin between a first portion of the sgRNA and a second portion of the sgRNA, wherein the first portion and the second portion together form a duplex portion.
Item 124 is the gRNA of any one of items 117-123, wherein the first internal linker substitutes for 2, 3, 4, 5, 6, 7, 8, 9, 10, 11, or 12 nucleotides of the upper stem region.
Item 125 is the gRNA of any one of items 117-124, wherein the first internal linker substitutes for a loop, or part thereof, of the upper stem region.
Item 126 is the gRNA of any one of items 117-125, wherein the first internal linker substitutes for the loop and the stem, or part thereof, of the upper stem region.
Item 127 is the gRNA of any one of items 117-126, wherein the first internal linker substitutes for 2, 3, or 4 nucleotides of the loop of the upper stem region.
Item 128 is the gRNA of any one of items 117-127, wherein the first internal linker substitutes for the loop of the upper stem region and at least 2, 4, 6, or 8 nucleotides of the stem of the upper stem region.
Item 129 is the gRNA of any one of items 117-128, wherein the first internal linker substitutes for the loop of the upper stem region and 1, 2, 3, or 4 base pairs of the stem of the upper stem region.
Item 130 is the gRNA of any one of items 117-129, wherein the first internal linker substitutes for all of the nucleotides constituting the loop of the upper stem region.
Item 131 is the gRNA of any one of items 117-130, wherein the first internal linker substitutes for all of the nucleotides constituting the loop and the stem of the upper stem region.
Item 132 is the gRNA of any one of items 117-131, wherein the second internal linker has a bridging length of about 6-18 atoms, optionally about 6-12 atoms.
Item 133 is the gRNA of any one of items 117-132, wherein the second internal linker substitutes for 2 nucleotides of the hairpin 1 of the sgRNA.
Item 134 is the gRNA of any one of items 117-133, wherein the second internal linker substitutes for 2 nucleotides of a stem region of the nexus region of the sgRNA.
Item 135 is the gRNA of any one of items 117-134, wherein the third internal linker has a bridging length of about 9-30, optionally about 12-21 atoms.
Item 136 is the gRNA of any one of items 117-135, wherein the third internal linker substitutes for 4, 6, 8, 10, or 12 nucleotides of the hairpin 2 of the gRNA.
Item 137 is the gRNA of any one of items 117-136, wherein the third linker substitutes for 1, 2, 3, 4, or 5 base pairs of the hairpin 2 of the gRNA.
Item 138 is the gRNA of any one of items 117-137, wherein the third internal linker substitutes for a loop, or part thereof, of the hairpin 2.
Item 139 is the gRNA of any one of items 117-138, wherein the third internal linker substitutes for the loop and the stem, or part thereof, of the hairpin 2.
Item 140 is the gRNA of any one of items 117-139, wherein the third internal linker substitutes for 2, 3, or 4 nucleotides of the loop of the hairpin 2.
Item 141 is the gRNA of any one of items 117-140, wherein the third internal linker substitutes for the loop of the hairpin and at least 1 nucleotide of the stem of the hairpin 2.
Item 142 is the gRNA of any one of items 117-141, wherein the third internal linker substitutes for the loop of the hairpin and 2, 4, or 6 nucleotides of the stem of the hairpin 2.
Item 143 is the gRNA of any one of items 117-142, wherein the third internal linker in the repeat-anti-repeat region substitutes for the loop of the hairpin and 1, 2, or 3 base pairs of the stem of the hairpin 2.
Item 144 is the gRNA of any one of items 117-143, wherein the third internal linker substitutes for all of the nucleotides constituting the loop of the hairpin 2.
Item 145 is the gRNA of any one of items 117-144, wherein the third internal linker is in a hairpin between a first portion of the sgRNA and a second portion of the sgRNA, wherein the first portion and the second portion together form a duplex portion.
Item 146 is the gRNA of any one of items 117-145, wherein the gRNA is a S. aureus Cas9 (SauCas9) guide RNA, and does not include the third internal linker.
Item 147 is the gRNA of any one of items 117-146, wherein the gRNA is a C. diphtheriae Cas9 (CdiCas9) guide RNA, an S. thermophilus Cas9 (St1Cas9) guide RNA, or an Acidothermus cellulolyticus Cas9 (AceCas9) guide RNA.
Item 148 is the gRNA of any one of items 117-147, wherein the sgRNA comprises a sequence of SEQ ID NO: 202.
Item 149 is the gRNA of item 148, wherein 22, 3 or 4 of nucleotides 35-38 are substituted for the first internal linker relative SEQ ID NO: 202.
Item 150 is the gRNA of any one of items 148-149, wherein nucleotides 34-39 are substituted for the first internal linker relative SEQ ID NO: 202.
Item 151 is the gRNA of any one of items 148-150, wherein nucleotides 33-40 are substituted for the first internal linker relative SEQ ID NO: 202.
Item 152 is the gRNA of any one of items 148-151, wherein nucleotides 32-41 are substituted for the first internal linker relative SEQ ID NO: 202.
Item 153 is the gRNA of any one of items 148-152, wherein nucleotides 31-42 are substituted for the first internal linker relative SEQ ID NO: 202.
Item 154 is the gRNA of any one of items 148-153, wherein nucleotide 61-62 are substituted for the second internal linker relative SEQ ID NO: 202.
Item 155 is the gRNA of any one of items 148-154, wherein 2, 3, or 4 of nucleotides 84-87 are substituted for the third internal linker relative SEQ ID NO: 202.
Item 156 is the gRNA of any one of items 148-155, wherein nucleotides 83-88 are substituted for the third internal linker relative SEQ ID NO: 202.
Item 157 is the gRNA of any one of items 148-156, wherein nucleotides 82-89 are substituted for the third internal linker relative SEQ ID NO: 202.
Item 158 is the gRNA of any one of items 148-157, wherein nucleotides 81-90 are substituted for the third internal linker relative SEQ ID NO: 202.
Item 159 is the gRNA of any one of items 148-158, wherein nucleotides 97-100 are deleted relative SEQ ID NO: 202.
Item 160 is a guide RNA (gRNA) comprising a guide region and a conserved portion 3′ to the guide region, wherein the conserved portion comprises a repeat-anti-repeat region, a hairpin 1 region, and a hairpin 2 region, and comprises a first internal linker substituting for at least 2 nucleotides of the repeat-anti-repeat region and a second internal linker substituting for at least 2 nucleotides of the hairpin 2.
Item 161 is the gRNA of item 160, wherein the first internal linker has a bridging length of about 9-30 atoms, optionally about 15-21 atoms.
Item 162 is the gRNA of any one of items 160-161, wherein the first internal linker substitutes for 2, 3, 4, 5, 6, 7, 8, 9, 10, 11, 12, 13, 14, 15, 16, 17, or 18 nucleotides of the repeat-anti-repeat region of the gRNA.
Item 163 is the gRNA of any one of items 160-162, wherein the first internal linker is in a hairpin between a first portion of the sgRNA and a second portion of the repeat-anti-repeat region, wherein the first portion and the second portion together form a duplex portion.
Item 164 is the gRNA of any one of items 160-163, wherein the first internal linker substitutes for a loop, or part thereof, of the hairpin of the repeat-anti-repeat region.
Item 165 is the gRNA of any one of items 160-164, wherein the first internal linker substitutes for the loop and the stem, or part thereof, of the hairpin of the repeat-anti-repeat region.
Item 166 is the gRNA of any one of items 160-165, wherein the first internal linker substitutes for 1, 2, 3, or 4 nucleotides of the loop of the hairpin of the repeat-anti-repeat region.
Item 167 is the gRNA of any one of items 160-166, wherein the first internal linker substitutes for the loop of the hairpin and at least 1, 2, 3, 4, 5, 6, 7, 8, 9, 10, 11, 12, 13, or 14 nucleotides of the upper stem of the hairpin of the repeat-anti-repeat region.
Item 168 is the gRNA of any one of items 160-167, wherein the first internal linker substitutes for the loop of the hairpin and 1, 2, 3, 4, 5, 6, or 7 base pairs of the upper stem of the hairpin of the repeat-anti-repeat region.
Item 169 is the gRNA of any one of items 160-168, wherein the first internal linker substitutes for all of the nucleotides constituting the loop of the hairpin of the repeat-anti-repeat region.
Item 170 is the gRNA of any one of items 160-169, wherein the first internal linker substitutes for all of the nucleotides constituting the loop and the upper stem of the hairpin of the repeat-anti-repeat region.
Item 171 is the gRNA of any one of items 160-169, wherein the first internal linker substitutes for all of the nucleotides constituting the loop of the repeat-anti-repeat region; and the upper stem of the hairpin of the repeat-anti-repeat region comprises at least one base pair, or no more than one, two, or three base pairs.
Item 172 is the gRNA of any one of items 160-171, wherein the second internal linker has a bridging length of about 9-30, optionally about 12-21 atoms.
Item 173 is the gRNA of any one of items 160-172, wherein the second internal linker substitutes for 2, 3, 4, 5, 6, 7, 8, 9, or 10 nucleotides of the hairpin 2 of the gRNA.
Item 174 is the gRNA of any one of items 160-173, wherein the second internal linker substitutes for a loop region of the hairpin 2.
Item 175 is the gRNA of any one of items 160-174, wherein the second internal linker substitutes for a loop region and part of a stem region of the hairpin 2.
Item 176 is the gRNA of any one of items 160-175, wherein the second internal linker substitutes for a loop, or part thereof, of the hairpin 2.
Item 177 is the gRNA of any one of items 160-176, wherein the second internal linker substitutes for the loop and the stem, or part thereof, of the hairpin 2.
Item 178 is the gRNA of any one of items 160-177, wherein the second internal linker substitutes for 2, 3, or 4 nucleotides of the loop of the hairpin 2.
Item 179 is the gRNA of any one of items 160-178, wherein the second internal linker substitutes for all of the nucleotides constituting the loop of the hairpin 2.
Item 180 is the gRNA of any one of items 160-179, wherein the second internal linker substitutes for the loop of the hairpin 2 and at least 1, 2, 3, 4, 5, or 6 nucleotides of the stem of the hairpin 2.
Item 181 is the gRNA of any one of items 160-180, wherein the second internal linker substitutes for the loop of the hairpin and 1, 2, or 3 base pairs of the stem of the hairpin 2
Item 182 is the gRNA of any one of items 160-181, wherein the gRNA is a St1Cas9 guide RNA.
Item 183 is the gRNA of any one of items 160-182, wherein the sgRNA comprises a sequence of SEQ ID NO: 204.
Item 184 is the gRNA of item 183, wherein nucleotides 41-44 are substituted for the first internal linker relative SEQ ID NO: 204.
Item 185 is the gRNA of any one of items 183-184, wherein nucleotides 101-103 are substituted for the second internal linker relative SEQ ID NO: 204.
Item 186 is the gRNA of any one of items 183-185, wherein nucleotides 100-104 are substituted for the second internal linker relative SEQ ID NO: 204.
Item 187 is the gRNA of any one of items 183-186, wherein nucleotides 99-105 are substituted for the second internal linker relative SEQ ID NO: 204.
Item 188 is the gRNA of any one of items 183-187, wherein nucleotides 98-106 are substituted for the second internal linker relative SEQ ID NO: 204.
Item 189 is the gRNA of any one of items 183-188, wherein 2-18 nucleotides within nucleotides 94-111 are substituted relative to SEQ ID NO: 204.
Item 190 is a guide RNA (gRNA) comprising a guide region and a conserved portion 3′ to the guide region, wherein the conserved portion comprises a repeat-anti-repeat region and a hairpin region, and comprises an internal linker substituting for at least 2 nucleotides of the repeat-anti-repeat region.
Item 191 is the gRNA of item 190, wherein the first internal linker has a bridging length of about 9-30 atoms, optionally about 12-21 atoms.
Item 192 is the gRNA of any one of items 190 or 191, wherein the first internal linker substitutes for 2, 3, 4, 5, 6, 7, 8, 9, 10, 11, or 12 nucleotides of the repeat-anti-repeat region of the gRNA.
Item 193 is the gRNA of any one of items 190-192, wherein the first internal linker is in a hairpin between a first portion of the sgRNA and a second portion of the repeat-anti-repeat region, wherein the first portion and the second portion together form a duplex portion.
Item 194 is the gRNA of any one of items 190-193, wherein the gRNA is a C. jejuni Cas9 (CjeCas9) guide RNA.
Item 195 is the gRNA of any one of items 190-194, wherein the gRNA is a CjeCas9 guide RNA and the internal linker is present only in the repeat-anti-repeat region of the gRNA.
Item 196 is the gRNA of any one of items 190-195, wherein the sgRNA comprises a sequence of SEQ ID NO: 207.
Item 197 is the gRNA of item 196, wherein nucleotides 33-36 are substituted for the internal linker relative SEQ ID NO: 207.
Item 198 is the gRNA of any one of items 196-197, wherein 1, 2, 3, 4, 5 or 6 base pairs of nucleotides 27-32 and 37-42 are substituted for the internal linker relative SEQ ID NO: 207.
Item 199 is the gRNA of any one of items 190-193, wherein the gRNA is a Francisella novicida Cas9 (FnoCas9) guide RNA.
Item 200 is the gRNA of item 199, wherein the sgRNA comprises a sequence of SEQ ID NO: 208.
Item 201 is the gRNA of item 200, wherein 2, 3 or 4 of nucleotides 40-43 are substituted for the internal linker relative SEQ ID NO: 208.
Item 202 is the gRNA of any one of items 200-201, wherein nucleotides 39-44 are substituted for the internal linker relative SEQ ID NO: 208.
Item 203 is a guide RNA (gRNA) comprising a repeat-anti-repeat region, and an internal linker substituting for at least 2 nucleotides of the repeat-anti-repeat region.
Item 204 is the gRNA of item 203, wherein the internal linker has a bridging length of about 9-30 atoms, optionally about 15-21 atoms.
Item 205 is the gRNA of any one of items 203-204, wherein the internal linker substitutes for 2, 3, 4, 5, or 6 nucleotides of the repeat-anti-repeat region of the gRNA.
Item 206 is the composition of any one of items 203-205, wherein the gRNA is a Cpf1 guide RNA.
Item 207 is the composition of item 206, wherein the Cpf1 guide RNA is a Lachnospiraceae bacterium Cpf1 (LbCpf1) guide RNA, or a Acidaminococcus sp. Cpf1 (AsCpf1) guide RNA.
Item 208 is the gRNA of any one of items 203-207, wherein the sgRNA comprises a sequence of SEQ ID NO: 209 and nucleotides 11-14, or 12-15, or optionally 12-14, are substituted for the internal linker relative SEQ ID NO: 209.
Item 209 is the composition of any one of item 203-205, wherein the guide RNA is an Eubacterium siraeum (EsCas13d) guide RNA.
Item 210 is the gRNA of any one of items 203-205, and 209, wherein the sgRNA comprises a sequence of SEQ ID NO: 210 and nucleotides 9-16, or optionally 10-15, or at least 2 nucleotides thereof; are substituted for the internal linker relative to SEQ ID NO: 210.
Item N211 is the gRNA of item 1, wherein the internal linker is a first internal linker, second internal linker, or third internal linker; and the gRNA comprises a guide region and a conserved region comprising one or more of:
(a) a shortened repeat/anti-repeat region, wherein the shortened repeat/anti-repeat region lacks 2-24 nucleotides, wherein
(i) one or more of nucleotides 37-64 is deleted and optionally substituted relative to SEQ ID NO: 500; and
(ii) nucleotide 36 is linked to nucleotide 65 by (i) a first internal linker that alone or in combination with nucleotides substitutes for 4 nucleotides, or (ii) at least 4 nucleotides; or
   (b) a shortened hairpin 1 region, wherein the shortened hairpin 1 lacks 2-10, optionally 2-8 nucleotides, wherein
(i) one or more of nucleotides 82-95 is deleted and optionally substituted relative to SEQ ID NO: 500; and
(ii) nucleotide 81 is linked to nucleotide 96 by (i) a second internal linker that alone or in combination with nucleotides substitutes for 4 nucleotides, or (ii) at least 4 nucleotides; or
   (c) a shortened hairpin 2 region, wherein the shortened hairpin 2 lacks 2-18, optionally 2-16 nucleotides, wherein
(i) one or more of nucleotides 113-134 is deleted and optionally substituted relative to SEQ ID NO: 500; and
   (ii) nucleotide 112 is linked to nucleotide 135 by (i) a third internal linker that alone or in combination with nucleotides substitutes for 4 nucleotides, or (ii) at least 4 nucleotides;
wherein one or both nucleotides 144-145 are optionally deleted as compared to SEQ ID NO: 500.

Item N212 is a guide RNA (gRNA) comprising a guide region and a conserved region comprising one or more of:
(a) a shortened repeat/anti-repeat region, wherein the shortened repeat/anti-repeat region lacks 2-24 nucleotides, wherein
(i) one or more of nucleotides 37-64 is deleted and optionally substituted relative to SEQ ID NO: 500; and
(ii) nucleotide 36 is linked to nucleotide 65 by (i) a first internal linker that alone or in combination with nucleotides substitutes for 4 nucleotides, or (ii) at least 4 nucleotides; or
   (b) a shortened hairpin 1 region, wherein the shortened hairpin 1 lacks 2-10, optionally 2-8 nucleotides, wherein
(i) one or more of nucleotides 82-95 is deleted and optionally substituted relative to SEQ ID NO: 500; and
(ii) nucleotide 81 is linked to nucleotide 96 by (i) a second internal linker that alone or in combination with nucleotides substitutes for 4 nucleotides, or (ii) at least 4 nucleotides; or
   (c) a shortened hairpin 2 region, wherein the shortened hairpin 2 lacks 2-18, optionally 2-16 nucleotides, wherein
(i) one or more of nucleotides 113-134 is deleted and optionally substituted relative to SEQ ID NO: 500; and
   (ii) nucleotide 112 is linked to nucleotide 135 by (i) a third internal linker that alone or in combination with nucleotides substitutes for 4 nucleotides, or (ii) at least 4 nucleotides;
wherein one or both nucleotides 144-145 are optionally deleted as compared to SEQ ID NO: 500;
wherein the gRNA comprises at least one of the first internal linker, the second internal linker, and the third internal linker.
Item N213 is the gRNA of item N211 or N212, wherein the gRNA comprises at least two of the first internal linker, the second internal linker, and the third internal linker.
Item N214 is the gRNA of any one of items N211-N213, wherein the gRNA comprises the first internal linker, the second internal linker, and the third internal linker.
Item N215 is the gRNA of any one of items N211-N214, wherein at least 10 nucleotides are modified nucleotides.
Item N216 is the gRNA of any one of items N211-N215, wherein the guide region has (i) an insertion of one nucleotide or a deletion of 1-4 nucleotides within positions 1-24 relative to SEQ ID NO: 500, or (ii) a length of 24 nucleotides.
Item N217 is the gRNA of any one of items N211-N216, wherein the guide region has a length of 25, 24, 23, 22, 21, or 20 nucleotides, optionally wherein the guide region has a length of 25, 24, 23, or 22 nucleotides at positions 1-24 of SEQ ID NO: 500.
Item N218 is the gRNA of item N217, wherein the guide region has a length of 23 or 24 nucleotides at positions 1-24 of SEQ ID NO: 500.
Item N219 is the gRNA of any one of items N211-N218, wherein the gRNA further comprises a 3′ tail.
Item N220 is the gRNA of item N219, wherein the 3′ tail comprises 1, 2, 3, 4, 5, 6, 7, 8, 9, or 10 nucleotides.
Item N221 is the gRNA of item N220, wherein the 3′ tail comprises 1, 2, 3, 4, or 5 nucleotides.
Item N222 is the gRNA of any one of items N219-N221, wherein the 3′ tail terminates with a nucleotide comprising a uracil or a modified uracil.
Item N223 is the gRNA of any one of items N219-N222, wherein the 3′ tail is 1 nucleotide in length.
Item N224 is the gRNA of any one of items N219-N223, wherein the 3′ tail consists of a nucleotide comprising a uracil or a modified uracil.
Item N225 is the gRNA of any one of items N219-N224, wherein the 3′ tail comprises a modification of any one or more of the nucleotides present in the 3′ tail.
Item N226 is the gRNA of any one of items N219-N225, wherein the modification of the 3′ tail is one or more of 2′-O-methyl (2′-OMe) modified nucleotide and a phosphorothioate (PS) linkage between nucleotides.
Item N227 is the gRNA of any one of the preceding items N219-226, wherein the 3′ tail is fully modified.
Item N228 is the gRNA of any one of items N211-N227, wherein the 3′ nucleotide of the gRNA is a nucleotide comprising a uracil or a modified uracil.
Item N229 is the gRNA of any one of items N211-N228, wherein one or more of nucleotides 144 and 145 are deleted relative to SEQ ID NO: 500.
Item N230 is the gRNA of any one of items N211-N229, wherein both nucleotides 144 and 145 are deleted relative to SEQ ID NO: 500.
Item N231 is the gRNA of any one of items N211-N218, wherein the gRNA does not comprise a 3′ tail.
Item N232 is the gRNA of any one of items N211-N231, wherein the shortened repeat/anti-repeat region lacks 2-28 nucleotides.
Item N233 is the gRNA of any one of items N211-N232, wherein the shortened repeat/anti-repeat region has a length of 24, 25, 26, 27, 28, 29, 30, 31, 32, 33, 34, 35, 36, 37, 38, 39, 40, 41, 42, 43, 44, 45, 46, 47, 48, 49, or 50 nucleotides.
Item N234 is the gRNA of any one of items N211-N233, wherein the shortened repeat/anti-repeat region lacks 12-28, optionally 18-24 nucleotides.
Item N235 is the gRNA of any one of items N211-N234, wherein the shortened repeat/anti-repeat region has a length of 24, 25, 26, 27, 28, 29, 30, 31, 32, 33, 34, 35, 36, 37, 38, 39, or 40 nucleotides.
Item N236 is the gRNA of any one of items N211-N235, wherein the shortened repeat/anti-repeat region has a length of 24, 25, 26, 27, 28, 29, 30, 31, 32, 33, or 34 nucleotides.
Item N237 is the gRNA of any one of items N211-N236, wherein nucleotides 37-64 of SEQ ID NO: 500 form the upper stem, and one or more base pairs of the upper stem of the shortened repeat/anti-repeat region are deleted.
Item N238 is the gRNA of any one of items N211-N237, wherein the upper stem of the shortened repeat/anti-repeat region comprises no more than one, two, three, or four base pairs.
Item N239 is the gRNA of any one of items N211-N238, wherein all of positions 39-48 and all of positions 53-62 of the upper stem of the shortened repeat/anti-repeat region are deleted, and optionally nucleotide 38 or 63 is substituted.
Item N240 is the gRNA of any one of items N211-N239, wherein all of positions 38-63 of the upper stem of the shortened repeat/anti-repeat region are deleted, and optionally nucleotide 37 or 64 is substituted.
Item N241 is the gRNA of any one of items N211-N240, wherein all of nucleotides 37-64 of the upper stem of the shortened repeat/anti-repeat region are deleted, and optionally nucleotide 36 or 65 is substituted.
Item N242 is the gRNA of any one of items N211-N241, wherein the shortened repeat/anti-repeat region has a duplex portion 11 base paired nucleotides in length.
Item N243 is the gRNA of any one of items N211-N242, wherein the shortened repeat/anti-repeat region has a single duplex portion.
Item N244 is the gRNA of any one of items N211-N243, wherein the upper stem of the shortened repeat/anti-repeat region includes one or more substitution relative to SEQ ID NO: 500.
Item N245 is the gRNA of any one of items N211-N244, wherein the first internal linker substitutes for at least part of or for all of nucleotides 49-52.
Item N246 is the gRNA of any one of items N211-N245, wherein all of nucleotides 37-64 are deleted and the first linker directly links nucleotide 36 to nucleotide 65.
Item N247 is the gRNA of any one of items N211-N245, wherein all of nucleotides 38-63 are deleted and the first linker directly links nucleotide 37 to nucleotide 64.
Item N248 is the gRNA of any one of items N211-N245, wherein all of nucleotides 39-62 are deleted and the first linker directly links nucleotide 38 to nucleotide 63.
Item N249 is the gRNA of any one of items N211-N248, wherein the shortened repeat/anti-repeat region has 8-22 modified nucleotides.
Item N250 is the gRNA of any one of items N211-N249, wherein the shortened hairpin 1 region lacks 2-10, optionally 2-8 or 2-4 nucleotides.
Item N251 is the gRNA of any one of items N211-N250, wherein the shortened hairpin 1 region has a length of 9, 10, 11, 12, 13, 14, 15, 16, 17, 18, 19, 20 or 21 nucleotides.
Item N252 is the gRNA of any one of items N211-N251, wherein the shortened hairpin 1 region has duplex portion 4-8, optionally 7-8 base paired nucleotides in length.
Item N253 is the gRNA of any one of items N211-N252, wherein the shortened hairpin 1 region has a single duplex portion.
Item N254 is the gRNA of any one of items N211-N253, wherein one or two base pairs of the shortened hairpin 1 region are deleted.
Item N255 is the gRNA of any one of items N211-N254, wherein the stem of the shortened hairpin 1 region is seven or eight base paired nucleotides in length.
Item N256 is the gRNA of any one of items N211-N255, wherein one or more of positions 85-86 and one or more of nucleotides 91-92 of the shortened hairpin 1 region are deleted.
Item N257 is the gRNA of any one of items N211-N256, wherein nucleotides 86 and 91 of the shortened hairpin 1 region are deleted.
Item N258 is the gRNA of any one of items N211-N257, wherein one or more of nucleotides 82-95 of the shortened hairpin 1 region is substituted relative to SEQ ID NO: 500.
Item N259 is the gRNA of any one of items N211-N258, wherein the second internal linker substitutes for at least part of or for all of nucleotides 87-90.
Item N260 is the gRNA of any one of items N211-N259, wherein the second internal linker substitutes for at least part of or nucleotides 81-95.
Item N261 is the gRNA of any one of items N211-N260, wherein the shortened hairpin 1 region has 2-15 modified nucleotides.
Item N262 is the gRNA of any one of items N211-N261, wherein the shortened hairpin 2 region lacks 2-18, optionally 2-16 nucleotides.
Item N263 is the gRNA of any one of items N211-N262, wherein the shortened hairpin 2 region has a length of 22, 23, 24, 25, 26, 27, 28, 29, 30, 31, 32, 33, 34, 35, 36, 37, 38, 39, or 40 nucleotides.
Item N264 is the gRNA of any one of items N211-N263, wherein the shortened hairpin 2 region has a length of 24, 25, 26, 27, 28, 29, 30, 31, 32, 33 or 34, nucleotides.
Item N265 is the gRNA of any one of items N211-N264, wherein one or more of positions 113-121 and one or more of nucleotides 126-134 of the shortened hairpin 2 region are deleted.
Item N266 is the gRNA of any one of items N211-N265, wherein the shortened hairpin 2 region comprises an unpaired region.
Item N267 is the gRNA of any one of items N211-N266, wherein the shortened hairpin 2 region has two duplex portions.
Item N268 is the gRNA of item N267, wherein the shortened hairpin 2 region has a duplex portion of 4 base paired nucleotides in length.
Item N269 is the gRNA of items N267-268, wherein the shortened hairpin 2 region has a duplex portion of 4-8 base paired nucleotides in length.
Item N270 is the gRNA of items N267-269, wherein the shortened hairpin 2 region has a duplex portion of 4-6 base paired nucleotides in length.
Item N271 is the gRNA of any one of items N211-N270, wherein the upper stem of the shortened hairpin 2 region comprises one, two, three, or four base pairs.
Item N272 is the gRNA of any one of items N211-N271, wherein at least one pair of nucleotides 113 and 134, nucleotides 114 and 133, nucleotides 115 and 132, nucleotides 116 and 131, nucleotides 117 and 130, nucleotides 118 and 129, nucleotides 119 and 128, nucleotides 120 and 127, and nucleotides 121 and 126 are deleted.
Item N273 is the gRNA of any one of items N211-N272, wherein all of positions 113-121 and 126-134 of the shortened hairpin 2 region are deleted.
Item N274 is the gRNA of any one of items N211-N273, wherein one or more of nucleotides 113-134 of the shortened hairpin 2 region is substituted relative to SEQ ID NO: 1.
Item N275 is the gRNA of any one of items N211-N274, wherein the third internal linker substitutes for at least part of or for all of nucleotides 122-125.
Item N276 is the gRNA of any one of items N211-N275, wherein the third internal linker substitutes for at least part of or for all of nucleotides 112-135.
Item 211 is the gRNA of any one of items 1-210 and N1-N276, wherein the internal linker has a bridging length of about 6 Angstroms-37 Angstroms.
Item 212 is the gRNA of any one of items 1-211, wherein the internal linker comprises at least two ethylene glycol subunits covalently linked to each other.
Item 213 is the gRNA of any one of items 1-212, wherein the internal linker comprises 1-10 ethylene glycol subunits covalently linked to each other.
Item 214 is the gRNA of any one of items 1-213, wherein the internal linker comprises 3-10 ethylene glycol subunits covalently linked to each other.
Item 215 is the gRNA of any one of items 1-214, wherein the internal linker comprises 3-6 ethylene glycol subunits covalently linked to each other.
Item 216 is the gRNA of any one of items 1-215, wherein the internal linker comprises 3 ethylene glycol subunits covalently linked to each other.
Item 217 is the gRNA of any one of items 1-216, wherein the internal linker comprises 6 ethylene glycol subunits covalently linked to each other.
Item 218 is the gRNA of any one of items 1-217, wherein the internal linker comprises a structure of formula (I):

˜-L0-L1-L2-# (I)
wherein:
˜ indicates a bond to a 3′ substituent of the preceding nucleotide;
#indicates a bond to a 5′ substituent of the following nucleotide;
L0 is null or C₁₋₃ aliphatic;
L1 is -[E¹-(R¹)]ₘ—, where
each R¹ is independently a C₁₋₅ aliphatic group, optionally substituted with 1 or 2 E²,
each E¹ and E² are independently a hydrogen bond acceptor, or are each independently chosen from cyclic hydrocarbons, and heterocyclic hydrocarbons, and each m is 1, 2, 3, 4, 5, 6, 7, 8, 9, or 10; and
L2 is null, C₁₋₃ aliphatic, or is a hydrogen bond acceptor.
Item 219 is the gRNA of item 218, wherein m is 6, 7, 8, 9, or 10.
Item 220 is the gRNA of any one of items 218-219, wherein m is 1, 2, 3, 4 or 5.
Item 221 is the gRNA of any one of items 218-220, wherein m is 1, 2, or 3.
Item 222 is the gRNA of any one of items 218-221, wherein L0 is null.
Item 223 is the gRNA of any one of items 218-221, wherein L0 is —CH₂— or —CH₂CH₂—.
Item 224 is the gRNA of any one of items 218-223, wherein L2 is null.
Item 225 is the gRNA of any one of items 218-223, wherein L2 is —O—, —S—, —CH₂— or —CH₂CH₂—.
Item 226 is the gRNA of any one of items 218-225, wherein the number of atoms in the shortest chain of atoms on the pathway from ˜ to #in the structure of Formula (I) is 30 or less, 27 or less, 24 or less, 21 or less, or is 18 or less, or is 15 or less, or is 12 or less, or is 10 or less.
Item 227 is the gRNA of any one of items 218-226, wherein the number of atoms in the shortest chain of atoms on the pathway from ˜ to #in the structure of Formula (I) is from 6 to 30, optionally 9 to 30, optionally 9 to 21.
Item 228 is the gRNA of any one of items 218-227, wherein the number of atoms in the shortest chain of atoms on the pathway from ˜ to #in the structure of Formula (I) is 9.
Item 229 is the gRNA of any one of items 218-227, wherein the number of atoms in the shortest chain of atoms on the pathway from ˜ to #in the structure of Formula (I) is 18.
Item 230 is the gRNA of any one of items 218-229, wherein each C₁₋₃ aliphatic group and C₁₋₅ aliphatic group is saturated.
Item 231 is the gRNA of any one of items 218-229, wherein at least one C₁₋₅ aliphatic group is a
C₁₋₄ alkylene, or wherein at least two C₁₋₅ aliphatic groups are a C₁₋₄ alkylene, or wherein at least three C₁₋₅ aliphatic groups are a C₁₋₄ alkylene.
Item 232 is the gRNA of any one of items 218-231, wherein at least one R¹ is selected from —CH₂—, —CH₂CH₂—, —CH₂CH₂CH₂—, or —CH₂CH₂CH₂CH₂—.
Item 233 is the gRNA of any one of items 218-231, wherein each R¹ is independently selected from —CH₂—, —CH₂CH₂—, —CH₂CH₂CH₂—, or —CH₂CH₂CH₂CH₂—.
Item 234 is the gRNA of any one of items 218-233, wherein each R¹ is —CH₂CH₂—.
Item 235 is the gRNA of any one of items 218-234, wherein at least one C₁₋₅ aliphatic group is a
C₁₋₄ alkenylene, or wherein at least two C₁₋₅ aliphatic groups are a C₁₋₄ alkenylene, or wherein at least three C₁₋₅ aliphatic groups are a C₁₋₄ alkenylene.
Item 236 is the gRNA of any one of items 218-235, wherein at least one R¹ is selected from
—CHCH—, —CHCHCH₂—, or —CH₂CHCHCH₂—.
Item 237 is the gRNA of any one of items 218-236, wherein each E¹ is independently chosen from —O—, —S—, —NH—, —NR—, —C(O)—O—, —OC(O)O—, —C(O)—NR—, —OC(O)—NR—, —NC(O)—NR—,
—P(O)₂O—, —OP(O)₂O—, —OP(R)(O)O—, —OP(O)(S)O—, —S(O)₂—, cyclic hydrocarbons, and heterocyclic hydrocarbons.
Item 238 is the gRNA of any one of items 218-237, wherein each E¹ is independently chosen from —O—, —S—, —NH—, —NR—, —C(O)—O—, —OC(O)O—, —P(O)₂O—, —OP(O)₂O—, and —OP(R)(O)O.
Item 239 is the gRNA of any one of items 218-238, wherein each E¹ is —O—.
Item 240 is the gRNA of any one of items 218-238, wherein each E¹ is —S—.
Item 241 is the gRNA of any one of items 218-240, wherein at least one C₁₋₅ aliphatic group in R¹ is optionally substituted with one E².
Item 242 is the gRNA of any one of items 218-241, wherein each E² is independently chosen from —OH, —OR, —ROR, —SH, —SR, —C(O)—R, —C(O)—OR, —OC(O)—OR, —C(O)—H, —C(O)—OH,
—OPO₃, —PO₃, —RPO₃, —S(O)₂—R, —S(O)₂—OR, —RS(O)₂—R, —RS(O)₂—OR, —SO₃, cyclic hydrocarbons, and heterocyclic hydrocarbons.
Item 243 is the gRNA of any one of items 218-242, wherein each E² is independently chosen from —OH, —OR, —SH, —SR, —C(O)—R, —C(O)—OR, —OC(O)—OR, —OPO₃, —PO₃, —RPO₃, and —SO₃.
Item 244 is the gRNA of any one of items 218-243, wherein each E² is —OH or OR.
Item 245 is the gRNA of any one of items 218-243, wherein each E² is —SH or SR.
Item 246 is the gRNA of any one of items 218-245, wherein the internal linker comprises a PEG-linker.
Item 247 is the gRNA of any one of items 218-246, wherein the internal linker comprises a PEG-linker having from 1 to 10 ethylene glycol units.
Item 248 is the gRNA of any one of items 218-247, wherein the internal linker comprises a PEG-linker having from 3 to 6 ethylene glycol units.
Item 249 is the gRNA of any one of items 218-248, wherein the internal linker comprises a PEG-linker having 3 ethylene glycol units.
Item 250 is the gRNA of any one of items 218-248, wherein the internal linker comprises a PEG-linker having 6 ethylene glycol units.
Item 251 is the gRNA of any one of items 1-250, wherein the gRNA is a short guide RNA comprising a shortened conserved portion, and the internal linker substitutes for at least 2, 3, 4, 5, 6, 7, 8, 9, 10, 11, or 12 nucleotides.
Item 252 is the gRNA of any one of the preceding items, wherein the gRNA is a short-single guide RNA (short-sgRNA) comprising a conserved portion of an sgRNA comprising a hairpin region, wherein the hairpin region lacks at least 5-10 nucleotides.
Item 253 is the gRNA of any one of the preceding items, wherein the at least 5-10 lacking nucleotides are consecutive.
Item 254 is the gRNA of any one of the preceding items, wherein the at least 5-10 lacking nucleotides
i. are within hairpin 1;
ii. are within hairpin 1 and the “N” between hairpin 1 and hairpin 2;
iii. are within hairpin 1 and the two nucleotides immediately 3′ of hairpin 1;
iv. include at least a portion of hairpin 1;
v. are within hairpin 2;
vi. include at least a portion of hairpin 2;
vii. are within hairpin 1 and hairpin 2;
viii. include at least a portion of hairpin 1 and include the “N” between hairpin 1 and hairpin 2;
ix. include at least a portion of hairpin 2 and include the “N” between hairpin 1 and hairpin 2;
x. include at least a portion of hairpin 1, include the “N” between hairpin 1 and hairpin 2, and include at least a portion of hairpin 2;
xi. are within hairpin 1 or hairpin 2, optionally including the “N” between hairpin 1 and hairpin 2;
xii. are consecutive;
xiii. are consecutive and include the “N” between hairpin 1 and hairpin 2;
xiv. are consecutive and span at least a portion of hairpin 1 and a portion of hairpin 2;
xv. are consecutive and span at least a portion of hairpin 1 and the “N” between hairpin 1 and hairpin 2; or
xvi. are consecutive and span at least a portion of hairpin 1 and two nucleotides immediately 3′ of hairpin 1.
Item 255 is the gRNA of any one of the preceding items, wherein the gRNA is a short-single guide RNA (short-sgRNA) comprising a conserved portion of an sgRNA comprising a hairpin region, wherein the hairpin region lacks at least 5-10 nucleotides and wherein the short-sgRNA comprises a 5′ end modification or a 3′ end modification.
Item 256 is the gRNA of any one of the preceding items, wherein the at least 5-10 nucleotides comprise nucleotides 54-61 of SEQ ID NO:400, nucleotides 53-60 of SEQ ID NO:400; or nucleotides 54-58 of SEQ ID NO:400, optionally wherein the short-sgRNA comprises modifications at least H1-1 to H1-5 and H2-1 to H2-12.
Item 257 is the gRNA of any one of the preceding items, comprising a shortened hairpin 1 region or a substituted and optionally shortened hairpin 1 region, wherein
(i) at least one of the following pairs of nucleotides are substituted in the substituted and optionally shortened hairpin 1 with Watson-Crick pairing nucleotides: H1-1 and H1-12, H1-2 and H1-11, H1-3 and H1-10, or H1-4 and H1-9, and the hairpin 1 region optionally lacks
(aa) any one or two of H1-5 through H1-8,
(bb) one, two, or three of the following pairs of nucleotides: H1-1 and H1-12, H1-2 and H1-11, H1-3 and H1-10 or H1-4 and H1-9, or
(cc) 1-8 nucleotides of the hairpin 1 region; or
(ii) the shortened hairpin 1 region lacks 6-8 nucleotides, preferably 6 nucleotides; and
(A) one or more of positions H1-1, H1-2, or H1-3 is deleted or substituted relative to SEQ ID NO: 400 or
(B) one or more of positions H1-6 through H1-10 is substituted relative to SEQ ID NO: 400; or
(iii) the shortened hairpin 1 region lacks 5-10 nucleotides, preferably 5-6 nucleotides, and one or more of positions N18, H1-12, or n is substituted relative to SEQ ID NO: 400.
Item 258 is the gRNA of any one of the preceding items, comprising a shortened upper stem region, wherein the shortened upper stem region lacks 1-6 nucleotides and wherein the 6, 7, 8, 9, 10, or 11 nucleotides of the shortened upper stem region include less than or equal to 4 substitutions relative to SEQ ID NO: 400.
Item 259 is the gRNA of any one of the preceding items, comprising a substitution relative to SEQ ID NO: 400 at any one or more of LS6, LS7, US3, US10, B3, N7, N15, N17, H2-2 and H2-14, wherein the substituent nucleotide is neither a pyrimidine that is followed by an adenine, nor an adenine that is preceded by a pyrimidine.
Item 260 is the gRNA of any one of the preceding items, comprising an upper stem region, wherein the upper stem modification comprises a modification to any one or more of US1-US12 in the upper stem region.
Item 261 is the gRNA of any one of the preceding items, comprising a shortened upper stem region, wherein the shortened upper stem region lacks 1-6 nucleotides.
Item 262 is the gRNA of any one of the preceding items, wherein the gRNA comprises a modification.
Item 263 is the guide RNA of item 262, wherein the modification comprises a 2′-O-methyl (2′-O-Me) modified nucleotide, a 2′-F modified nucleotide, 2′-H modified nucleotide (DNA), a 2′-O,4′-C-ethylene modified nucleotides (ENA), locked nucleotide (LNA), or unlocked nucleotide (UNA).
Item 264 is the guide RNA of item 262 or 263, wherein the modification comprises a phosphorothioate (PS) bond between nucleotides.
Item 265 is the guide RNA of any one of items 262-264, wherein the guide RNA is a sgRNA and the modification, comprises a modification at one or more of the five nucleotides at the 5′ end of the guide RNA.
Item 266 is the guide RNA of any one of items 262-265, wherein the guide RNA is a sgRNA and the modification, comprises a modification at one or more of the five nucleotides at the 3′ end of the guide RNA.
Item 267 is the guide RNA of any one of items 262-266, wherein the guide RNA is a sgRNA and the modification, comprises a PS bond between each of the four nucleotides at the 5′ end of the guide RNA.
Item 268 is the guide RNA of any one of items 262-267, wherein the guide RNA is a sgRNA and the modification, comprises a PS bond between each of the four nucleotides at the 3′ end of the guide RNA.
Item 269 is the guide RNA of any one of items 262-268, wherein the guide RNA is a sgRNA and the modification, comprises a 2′-O-Me modified nucleotide at each of the first three nucleotides at the 5′ end of the guide RNA.
Item 270 is the guide RNA of any one of items 262-269, wherein the guide RNA is a sgRNA and the modification, comprises a 2′-O-Me modified nucleotide at each of the last three nucleotides at the 3′ end of the guide RNA.
Item 271 is the gRNA of any one of the preceding items, wherein the gRNA comprises a 3′ tail.
Item 272 is the gRNA of item 271, wherein the 3′ tail comprises at least 1-10 nucleotides.
Item 273 is the gRNA of any one of items 271-272, wherein the 3′ tail terminates with a nucleotide with a uracil base.
Item 274 is the gRNA of any one of items 271-273, wherein the 3′ tail is 1 nucleotide in length and is a nucleotide with a uracil base.
Item 275 is the gRNA of any one of the preceding items, wherein the 3′ nucleotide of the gRNA is a nucleotide with a uracil base.
Item 276 is the gRNA of any one of items 271-274, wherein the 3′ tail comprises a modification of any one or more of the nucleotides present in the 3′ tail.
Item 277 is the gRNA of item 276, wherein the 3′ tail is fully modified.
Item 278 is the gRNA of any one of items 1-270, wherein the gRNA does not comprise a 3′ tail.
Item 279 is the gRNA of any one of the preceding items, wherein the gRNA comprises a 3′ end modification.
Item 280 is the gRNA of any one of the preceding items, wherein the gRNA comprises a 5′ end modification and a 3′ end modification.
Item 281 is the gRNA of any one of items 279-280, wherein the 3′ or 5′ end modification comprises a protective end modification, optionally a modified nucleotide selected from a 2′-O-methyl (2′-OMe) modified nucleotide, a 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide, or a combination thereof.
Item 282 is the gRNA of any one of items 279-281, wherein the 3′ or 5′ end modification comprises or further comprises a 2′-O-methyl (2′-Ome) modified nucleotide.
Item 283 is the gRNA of any one of items 279-282, wherein the 3′ or 5′ end modification comprises or further comprises a 2′-fluoro (2′-F) modified nucleotide.
Item 284 is the gRNA of any one of items 279-283, wherein the 3′ or 5′ end modification comprises or further comprises a phosphorothioate (PS) linkage between nucleotides.
Item 285 is the gRNA of any one of items 279-284, wherein the 3′ or 5′ end modification comprises or further comprises an inverted abasic modified nucleotide.
Item 286 is the gRNA of any one of the preceding items, comprising a modification in a or the hairpin region.
Item 287 is the gRNA of item 286, comprising a modification in the hairpin region, wherein the modification in the hairpin region comprises a modified nucleotide selected from a 2′-O-methyl (2′-Ome) modified nucleotide, a 2′-fluoro (2′-F) modified nucleotide, a phosphorothioate (PS) linkage between nucleotides, or a combination thereof.
Item 288 is the gRNA of item 286 or 287, further comprising a 3′ end modification.
Item 289 is the gRNA of item 286 or 287, further comprising a 3′ end modification and a 5′ end modification.
Item 290 is the gRNA of item 286 or 287, further comprising a 5′ end modification.
Item 291 is the gRNA of any one of items 286-290, wherein the modification in the hairpin region comprises or further comprises a 2′-O-methyl (2′-Ome) modified nucleotide.
Item 292 is the gRNA of any one of items 286-291, wherein the modification in the hairpin region comprises or further comprises a 2′-fluoro (2′-F) modified nucleotide.
Item 293 is the gRNA of any one of the preceding items, comprising a modification in a or the upper stem region.
Item 294 is the gRNA of item 293, wherein the upper stem modification comprises any one or more of:
i. a modification of any one or more of US1-US12 in the upper stem region; and
ii. a modification of at least 1, 2, 3, 4, 5, 6, 7, 8, 9, 10, 11, or all 12 nucleotides in the upper stem region.
Item 295 is the gRNA of item 293, wherein the upper stem modification comprises one or more of:
i. a 2′-OMe modified nucleotide;
ii. a 2′-O-moe modified nucleotide;
iii. a 2′-F modified nucleotide;
iv. 2′-H modified nucleotide (DNA);
v. a 2′-0,4′-C-ethylene modified nucleotides (ENA);
vi. locked nucleotide (LNA);
vii. unlocked nucleotide (UNA); and
viii. combinations of one or more of (i.)-(iii.).
Item 296 is the gRNA of any one of the preceding items, wherein the modification comprises a YA modification.
Item 297 is the gRNA of any one of the preceding items, comprising a YA modification of one or more guide region YA sites.
Item 298 is the gRNA of any one of items 296-297, wherein the YA modification comprises a substitution of the pyrimidine of a YA site with a non-pyrimidine.
Item 299 is the gRNA of any one of items 296-297, wherein the YA modification comprises a substitution of the adenine of a YA site with a non-adenine.
Item 300 is the gRNA of any one of items 296-298, comprising a YA modification wherein the modification comprises 2′-fluoro, 2′-H, 2′-OMe, ENA, UNA, inosine, or PS modification.
Item 301 is the gRNA of any one of the preceding items, comprising a YA modification of one or more conserved region YA sites.
Item 302 is the gRNA of any one of the preceding items, wherein the YA modification comprises
(i) a 2′-OMe modification, optionally of the pyrimidine of the YA site;
(ii) a 2′-fluoro modification, optionally of the pyrimidine of the YA site; or
(iii) a PS modification, optionally of the pyrimidine of the YA site.
Item 303 is the gRNA of any one of items 61-302, comprising a nucleotide sequence having at least 99, 98, 97, 96, 95, 94, 93, 92, 91, 90, 85, 80, 75, or 70% identity to the nucleotide sequence of any one of SEQ ID NOs: 1-8, 20-75, 101-108, and 120-175.
Item 304 is the gRNA of any one of items 61-303, comprising a nucleotide sequence having at least 99, 98, 97, 96, 95, 94, 93, 92, 91, 90, 85, 80, 75, or 70% identity to the nucleotide sequence of any one of SEQ ID Nos: 1-8, 20-75, 101-108, and 120-175, wherein the modification at each nucleotide of the gRNA that corresponds to a nucleotide of the reference sequence identifier in Table 2A is identical to or equivalent to the modification shown in the reference sequence identifier in Table 2B.
Item 305 is a guide RNA (gRNA) comprising any of SEQ ID NOs: 1-8 and 20-75.
Item 306 is the gRNA of any one of the preceding items, including modifications set forth for a guide RNA in Table 2A or Table 2B.
Item 307 is a guide RNA (gRNA) comprising any one of SEQ ID NOs: 101-108 and 120-175, including the modifications of Table 2A or Table 2B.
Item 308 is the gRNA of any one of the preceding items, wherein the gRNA is associated with a lipid nanoparticle (LNP).
Item 309 is a composition comprising the gRNA of any one of the preceding items.
Item 310 is an LNP composition comprising a gRNA of any one of items 1-308.
Item 311 is a LNP composition comprising a gRNA of any one of items 63-116 and 211-308 and an mRNA encoding SpyCas9.
Item 312 is a LNP composition comprising a gRNA of any one of items 117-159 and 211-308 and an mRNA encoding SauCas9.
Item 313 is a LNP composition comprising a gRNA of any one of items 160-189 and 211-308 and an mRNA encoding St1Cas9.
Item 314 is a LNP composition comprising a gRNA of any one of items 190-202 and 211-308 and an mRNA encoding CjeCas9 or FnoCas9.
Item 315 is a LNP composition comprising a gRNA of any one of items 203-308 and an mRNA encoding AsCpf1, LbCpf1, or EsCas13d.
Item 316 is the LNP composition of any one of items 310-315, wherein the LNP comprises (9z,12z)-3-((4,4-bis(octyloxy)butanoyl)oxy)-2-((((3-(diethylamino)propoxy)carbonyl)oxy)methyl)propyl octadeca-9,12-dienoate or nonyl 8-((7,7-bis(octyloxy)heptyl)(2-hydroxyethyl)amino)octanoate.
Item 317 is the composition of any one of items 310-316, wherein the LNP comprises a molar ratio of a cationic lipid amine to RNA phosphate (N:P) of about 4.5-6.5, optionally the N:P of about 6.0.
Item 318 is the composition of item 309-317, wherein the nuclease comprises a protein or a nucleic acid encoding the nuclease.
Item 319 is the composition of item 318, wherein the nuclease is a Cas nuclease.
Item 320 is the composition of item 319, wherein the Cas nuclease is a Cas9.
Item 321 is the composition of item 320, wherein the Cas9 is S. pyogenes Cas9 (SpyCas9).
Item 322 is the composition of item 320, wherein the Cas9 is S. aureus Cas9 (SauCas9).
Item 323 is the composition of item 320, wherein the Cas9 is C. diphtheriae Cas9 (CdiCas9).
Item 324 is the composition of item 320, wherein the Cas9 is Streptococcus thermophilus Cas9 (St1Cas9).
Item 325 is the composition of item 320, wherein the Cas9 is A. cellulolyticus Cas9 (AceCas9).
Item 326 is the composition of item 320, wherein the Cas9 is C. jejuni Cas9 (CjeCas9).
Item 327 is the composition of item 320, wherein the Cas9 is R. palustris Cas9 (RpaCas9).
Item 328 is the composition of item 320, wherein the Cas9 is R. rubrum Cas9 (RruCas9).
Item 329 is the composition of item 320, wherein the Cas9 is A. naeslundii Cas9 (AnaCas9).
Item 330 is the composition of item 320, wherein the Cas9 is Francisella novicida Cas9 (FnoCas9).
Item 330.1 is the composition of item 320, wherein the Cas protein is a Neisseria meningitidis Cas9 (NmeCas9).
Item 331 is the composition of item 319, wherein the Cas nuclease is a Cpf1.
Item 332 is the composition of item 331, wherein the Cpf1 is Lachnospiraceae bacterium Cpf1 (LbCpf1) or the Cpf1 is Acidaminococcus sp. Cpf1 (AsCpf1).
Item 333 is the composition of item 319, wherein the Cas protein is an Eubacterium siraeum Cas13d (EsCas13d).
Item 334 is the composition of any one of items 311-329, wherein the nuclease is a cleavase, a nickase, or a catalytically inactive nuclease, or is a fusion protein comprising a deaminase.
Item 335 is the composition of any one of items 311-334, wherein the nuclease is modified.
Item 336 is the composition of the immediately preceding item, wherein the modified nuclease comprises a nuclear localization signal (NLS).
Item 337 is the composition of item 311-336, wherein the nucleic acid encoding the nuclease is selected from:
a. a DNA coding sequence;
b. an mRNA with an open reading frame (ORF);
c. a coding sequence in an expression vector;
d. a coding sequence in a viral vector.
Item 338 is the composition of the immediately preceding item, wherein the mRNA comprises the sequence of any one of SEQ ID NOs: 321-323.
Item 339 is a pharmaceutical formulation comprising the gRNA of any one of items 1-308, or the composition of any one of items 309-338 and a pharmaceutically acceptable carrier.
Item 340 is a method of modifying a target DNA comprising, delivering to a cell any one or more of the following:
i. the gRNA of any one of items 1-308;
ii. the composition of any one of items 309-338; and
iii. the pharmaceutical formulation of item 339.
Item 341 is the method of item 340, wherein the gRNA comprises no more than 110, 115, 110, 105, 100, 95, 90, 85, 80, 75, 70, 65, 60, 55, 50, 45, or 40, nucleotides.
Item 342 is the gRNA of any one of items 1-308, the composition of items 309-338, or the pharmaceutical formulation of item 340 for use in preparing a medicament for treating a disease or disorder.
Item 343 is the use of the gRNA of any one of items 1-308, the composition of items 309-338, or the pharmaceutical formulation of item 340 in the manufacture of a medicament for treating a disease or disorder.
Item 344 is a chemically synthesized gRNA comprising an internal linker.
Item 345 is a composition comprising the gRNA of any one of items 1-308, wherein the composition does not comprise an unlinked portion of the gRNA.
Item 346 is a solid support covalently attached to the linker of the gRNA of any one of items 1-308.
Item 347 is a method of synthesizing a gRNA comprising an internal linker wherein it is a single synthetic process.
Item 348 is a method of synthesizing a gRNA wherein an internal linker incorporated in line during synthesis.
Item 349 is a method of synthesizing a gRNA using a series of sequential coupling reactions wherein the reactions comprise:
a) coupling reaction for covalent linkage of a first nucleotide to a second nucleotide;
b) a coupling reaction for covalent linkage of an internal linker to the second nucleotide; and
c) a coupling reaction for covalent linkage of a third nucleotide to the internal linker, wherein the coupling reaction for the covalent linkages are all the same.
Item 350 is the method of item 349, wherein covalent linkage is performed using phosphoramidite chemistry.
Item 351 is the gRNA, composition, formulation, method, or use of any one of the preceding items, wherein the gRNA is chemically synthesized.
Item 352 is the gRNA, composition, formulation, method, or use of any one of the preceding items, wherein the internal linker is incorporated into the gRNA via a coupling reaction during chemical synthesis of the gRNA.
Item 353 is the gRNA, composition, formulation, method, or use of any one of the preceding items, prepared by a process comprising addition of the internal linker by reacting a linker comprising a phosphoramidite moiety with a nucleoside residue.
Item 354 is the gRNA, composition, formulation, method, or use of the immediately preceding item, wherein the process further comprises reacting a nucleotide comprising a phosphoramidite moiety with the linker.
Item 355 is the gRNA, composition, formulation, method, or use of any one of the preceding items, wherein the internal linker is covalently joined to the adjacent nucleotide by a phosphodiester or a phosphorothioate bond.
Item 356 is the gRNA, composition, formulation, method, or use of any one of the preceding items, wherein no urea is present in the internal linker.
Item 357 is the gRNA, composition, formulation, method, or use of any one of the preceding items, wherein the internal linker is not in the repeat-anti-repeat region of the gRNA.
Item 358 is the gRNA, composition, formulation, method, or use of any one of the preceding items, wherein the gRNA comprises an internal linker that is not in a repeat-anti-repeat of the guide.
Item 359 is the gRNA, composition, formulation, method, or use of any one of the preceding items, wherein the gRNA is an sgRNA.
Item 360 is the gRNA, composition, formulation, method, or use of any one of the preceding items, wherein the internal linker bridges a duplex region and substitutes for 2-12 nucleotides.
Item 361 is the gRNA, composition, formulation, method, or use of any one of the preceding items, wherein the gRNA is made in a single synthesis.

We claim:

1. A guide RNA (gRNA) comprising an internal linker.

2. The gRNA of claim 1 wherein the internal linker has a bridging length of about 3-30 atoms, optionally 12-21 atoms, and the linker substitutes for at least 2 nucleotides of the gRNA.

3. The gRNA of claim 1 or 2, wherein the internal linker has a bridging length of about 6-18 atoms, optionally about 6-12 atoms, and the linker substitutes for at least 2 nucleotides of the gRNA.

4. The gRNA of any one of claims 1-3, wherein the internal linker substitutes for 2-12 nucleotides.

5. The gRNA of any one of claims 1-4, wherein the internal linker is in a repeat-anti-repeat region of the gRNA.

6. The gRNA of any one of claims 1-5, wherein the internal linker substitutes for at least 4 nucleotides of the repeat-anti-repeat region of the gRNA.

7. The gRNA of any one of claims 1-6, wherein the internal linker substitutes for up to 28 nucleotides of the repeat-anti-repeat region of the gRNA.

8. The gRNA of any one of claims 1-7, wherein the internal linker is in a hairpin region of the gRNA.

9. The gRNA of any one of claims 1-8, wherein the internal linker is in a nexus region of the gRNA.

10. The gRNA of any one of claims 1-9, wherein the internal linker is in a hairpin between a first portion of the gRNA and a second portion of the gRNA, wherein the first portion and the second portion together form a duplex portion.

11. The gRNA of any one of claims 1-10, wherein the internal linker in the repeat-anti-repeat region is in a hairpin between a first portion and a second portion of the repeat-anti-repeat region, wherein the first portion and the second portion together form a duplex portion.

12. The gRNA of any one of claims 1-11, wherein the internal linker substitutes for a hairpin of the gRNA.

13. The gRNA of any one of claims 1-12, wherein the gRNA is a single guide RNA (sgRNA).

14. A guide RNA (gRNA), wherein the gRNA is a single-guide RNA (sgRNA) comprising a guide region and a conserved portion at 3′ to the guide region, wherein the conserved portion comprises a repeat-anti-repeat region, a nexus region, a hairpin 1 region, and a hairpin 2 region, and comprises at least one of:
4) a first internal linker substituting for at least 2 nucleotides of an upper stem region of the repeat-anti-repeat region;
5) a second internal linker substituting for 1 or 2 nucleotides of the nexus region; and
6) a third internal linker substituting for at least 2 nucleotides of the hairpin 1.

15. The gRNA of claim 14, wherein the first internal linker has a bridging length of about 9-30 atoms, optionally about 15-21 atoms.

16. The gRNA of claim 14 or claim 15, wherein the first internal linker substitutes for a loop, or part thereof, of the upper stem region.

17. The gRNA of any one of claims 14-16, wherein the first internal linker substitutes for the loop and the stem, or part thereof, of the upper stem region.

18. The gRNA of any one of claims 14-17, wherein the first internal linker substitutes for all of the nucleotides constituting the loop of the upper stem region.

19. The gRNA of any one of claims 14-18, wherein the first internal linker substitutes for all of the nucleotides constituting the loop and the stem of the upper stem region.

20. The gRNA of any one of claims 14-19, wherein the second internal linker has a bridging length of about 6-18 atoms, optionally about 6-12 atoms.

21. The gRNA of any one of claims 14-20, wherein the third internal linker has a bridging length of about 9-30, optionally about 12-21 atoms.

22. The gRNA of any one of claims 14-21, wherein the third internal linker substitutes for a loop, or part thereof, of the hairpin 1.

23. The gRNA of any one of claims 14-21, wherein the third internal linker substitutes for the loop and the stem, or part thereof, of the hairpin 1.

24. The gRNA of any one of claims 14-23, wherein the third internal linker substitutes for all of the nucleotides constituting the loop of the hairpin 1.

25. The gRNA of any one of claims 14-23, wherein the third internal linker substitutes for all of the nucleotides constituting the loop and the stem of the hairpin 1.

26. The gRNA of any one of claims 14-25, wherein the hairpin 2 region of the sgRNA does not contain any internal linker.

27. The gRNA of any one of claims 14-26, wherein the sgRNA is an S. pyogenes Cas9 sgRNA.

28. The gRNA of any one of claims 14-27, wherein the sgRNA comprises a conserved portion comprising a sequence of SEQ ID NO: 400.

29. The gRNA of claim 28, wherein
1) 2, 3 or 4 of nucleotides 13-16 (US5-US8 of the upper stem region) are substituted for the first internal linker relative to SEQ ID NO: 400;
2) nucleotides 12-17 (US4-US9 of the upper stem region) are substituted for the first internal linker relative to SEQ ID NO: 400;
3) nucleotides to 11-18 (US3-US10 of the upper stem region) are substituted for the first internal linker relative to SEQ ID NO: 400;
4) nucleotides to 10-19 (US2-US11 of the upper stem region) are substituted for the first internal linker relative to SEQ ID NO: 400; or
5) nucleotides to 9-20 (US1-US12 of the upper stem region) are substituted for the first internal linker relative to SEQ ID NO: 400.

30. The gRNA of claim 28 or 29,
1) wherein nucleotide 36-37 (N6-N7 of the nexus region) are substituted for the second internal linker relative to SEQ ID NO: 400;
2) wherein 2, 3, or 4 of nucleotides 53-56 (H1-5-H1-8 of the hairpin 1) are substituted for the third internal linker relative to SEQ ID NO: 400;
3) wherein nucleotides 52-57 (H1-4-H1-9 of the hairpin 1) are substituted for the third internal linker relative to SEQ ID NO: 400;
4) wherein nucleotides 51-58 (H1-3-H1-10 of the hairpin 1) are substituted for the third internal linker relative to SEQ ID NO: 400; or
5) wherein nucleotides 50-59 (H1-1-H1-12 of the hairpin 1) are substituted for the third internal linker relative to SEQ ID NO: 400.

31. The gRNA of any one of claims 28-30, wherein nucleotides 77-80 are deleted relative to SEQ ID NO: 400.

32. The gRNA of any one of claims 14-27, wherein the sgRNA comprises a sequence of SEQ ID NO: 201.

33. The gRNA of claim 32, wherein
1) 2, 3 or 4 of nucleotides 33-36 are substituted for the first internal linker relative to SEQ ID NO: 201.
2) nucleotides 32-37 are substituted for the first internal linker relative to SEQ ID NO: 201;
3) nucleotides 31-38 are substituted for the first internal linker relative to SEQ ID NO: 201;
4) nucleotides 30-39 are substituted for the first internal linker relative to SEQ ID NO: 201; or
5) nucleotides 29-40 are substituted for the first internal linker relative to SEQ ID NO: 201.

34. The gRNA of claim 32 or 33, wherein
1) nucleotide 55-56 are substituted for the second internal linker relative SEQ ID NO: 201.
2) 2, 3, or 4 of nucleotides 50-53 are substituted for the third internal linker relative to SEQ ID NO: 201.
3) nucleotides 49-54 are substituted for the third internal linker relative to SEQ ID NO: 201.

35. The gRNA of any one of claims 32-34, wherein nucleotides 77-80 are deleted relative to SEQ ID NO: 201.

36. The gRNA of claim 1, wherein the internal linker is a first internal linker, second internal linker, or third internal linker; and the gRNA comprises a guide region and a conserved region comprising one or more of:
(a) a shortened repeat/anti-repeat region, wherein the shortened repeat/anti-repeat region lacks 2-24 nucleotides, wherein
(i) one or more of nucleotides 37-64 is deleted and optionally substituted relative to SEQ ID NO: 500; and
(ii) nucleotide 36 is linked to nucleotide 65 by (i) a first internal linker that alone or in combination with nucleotides substitutes for 4 nucleotides, or (ii) at least 4 nucleotides; or
(b) a shortened hairpin 1 region, wherein the shortened hairpin 1 lacks 2-10, optionally 2-8 nucleotides, wherein
(i) one or more of nucleotides 82-95 is deleted and optionally substituted relative to SEQ ID NO: 500; and
(ii) nucleotide 81 is linked to nucleotide 96 by (i) a second internal linker that alone or in combination with nucleotides substitutes for 4 nucleotides, or (ii) at least 4 nucleotides; or
(c) a shortened hairpin 2 region, wherein the shortened hairpin 2 lacks 2-18, optionally 2-16 nucleotides, wherein
(i) one or more of nucleotides 113-134 is deleted and optionally substituted relative to SEQ ID NO: 500; and
(ii) nucleotide 112 is linked to nucleotide 135 by (i) a third internal linker that alone or in combination with nucleotides substitutes for 4 nucleotides, or (ii) at least 4 nucleotides;
wherein one or both nucleotides 144-145 are optionally deleted as compared to SEQ ID NO: 500.

37. A guide RNA (gRNA) comprising a guide region and a conserved region comprising one or more of:
(a) a shortened repeat/anti-repeat region, wherein the shortened repeat/anti-repeat region lacks 2-24 nucleotides, wherein
(i) one or more of nucleotides 37-64 is deleted and optionally substituted relative to SEQ ID NO: 500; and
(ii) nucleotide 36 is linked to nucleotide 65 by (i) a first internal linker that alone or in combination with nucleotides substitutes for 4 nucleotides, or (ii) at least 4 nucleotides; or
(b) a shortened hairpin 1 region, wherein the shortened hairpin 1 lacks 2-10, optionally 2-8 nucleotides, wherein
(i) one or more of nucleotides 82-95 is deleted and optionally substituted relative to SEQ ID NO: 500; and
(ii) nucleotide 81 is linked to nucleotide 96 by (i) a second internal linker that alone or in combination with nucleotides substitutes for 4 nucleotides, or (ii) at least 4 nucleotides; or
(c) a shortened hairpin 2 region, wherein the shortened hairpin 2 lacks 2-18, optionally 2-16 nucleotides, wherein
(i) one or more of nucleotides 113-134 is deleted and optionally substituted relative to SEQ ID NO: 500; and
(ii) nucleotide 112 is linked to nucleotide 135 by (i) a third internal linker that alone or in combination with nucleotides substitutes for 4 nucleotides, or (ii) at least 4 nucleotides;
wherein one or both nucleotides 144-145 are optionally deleted as compared to SEQ ID NO: 500;
wherein the gRNA comprises at least one of the first internal linker, the second internal linker, and the third internal linker.

38. The gRNA of claim 36 or claim 37, wherein at least 10 nucleotides are modified nucleotides.

39. The gRNA of any one of claims 36-38, wherein the guide region has (i) an insertion of one nucleotide or a deletion of 1-4 nucleotides within positions 1-24 relative to SEQ ID NO: 500, or (ii) a length of 24 nucleotides.

40. The gRNA of any one of claims 36-39, wherein the guide region has a length of 23 or 24 nucleotides at positions 1-24 of SEQ ID NO: 500.

41. The gRNA of any one of claims 36-40, wherein one or more of nucleotides 144 and 145 are deleted relative to SEQ ID NO: 500.

42. The gRNA of any one of claims 36-41, wherein both nucleotides 144 and 145 are deleted relative to SEQ ID NO: 500.

43. The gRNA of any one of claims 36-42, wherein the shortened repeat/anti-repeat region lacks 2-28 nucleotides.

44. The gRNA of any one of claims 36-43, wherein the shortened repeat/anti-repeat region lacks 12-28, optionally 18-24 nucleotides.

45. The gRNA of any one of claims 36-44, wherein nucleotides 37-64 of SEQ ID NO: 500 form the upper stem, and one or more base pairs of the upper stem of the shortened repeat/anti-repeat region are deleted.

46. The gRNA of any one of claims 36-45, wherein the upper stem of the shortened repeat/anti-repeat region comprises no more than one, two, three, or four base pairs.

47. The gRNA of any one of claims 36-46, wherein the shortened repeat/anti-repeat region has a duplex portion 11 base paired nucleotides in length.

48. The gRNA of any one of claims 36-47, wherein the shortened repeat/anti-repeat region has a single duplex portion.

49. The gRNA of any one of claims 36-48, wherein the upper stem of the shortened repeat/anti-repeat region includes one or more substitution relative to SEQ ID NO: 500.

50. The gRNA of any one of claims 36-49, wherein the first internal linker substitutes for at least part of or for all of nucleotides 49-52.

51. The gRNA of any one of claims 36-50, wherein the shortened hairpin 1 region has duplex portion 4-8, optionally 7-8 base paired nucleotides in length.

52. The gRNA of any one of claims 36-51, wherein the shortened hairpin 1 region has a single duplex portion.

53. The gRNA of any one of claims 36-52, wherein one or two base pairs of the shortened hairpin 1 region are deleted.

54. The gRNA of any one of claims 36-53, wherein the stem of the shortened hairpin 1 region is seven or eight base paired nucleotides in length.

55. The gRNA of any one of claims 36-54, wherein one or more of positions 85-86 and one or more of nucleotides 91-92 of the shortened hairpin 1 region are deleted.

56. The gRNA of any one of claims 36-55, wherein one or more of nucleotides 82-95 of the shortened hairpin 1 region is substituted relative to SEQ ID NO: 500.

57. The gRNA of any one of claims 36-56, wherein the second internal linker substitutes for at least part of or for all of nucleotides 87-90.

58. The gRNA of any one of claims 36-56, wherein the second internal linker substitutes for at least part of or for all nucleotides 81-95.

59. The gRNA of any one of claims 36-58, wherein the shortened hairpin 1 region has 2-15 modified nucleotides.

60. The gRNA of any one of claims 36-59, wherein the shortened hairpin 2 region lacks 2-18, optionally 2-16 nucleotides.

61. The gRNA of any one of claims 36-60, wherein one or more of positions 113-121 and one or more of nucleotides 126-134 of the shortened hairpin 2 region are deleted.

62. The gRNA of any one of claims 36-61, wherein the shortened hairpin 2 region comprises an unpaired region.

63. The gRNA of any one of claims 36-62, wherein the shortened hairpin 2 region has two duplex portions.

64. The gRNA of claim 63, wherein the shortened hairpin 2 region has a duplex portion of 4 base paired nucleotides in length.

65. The gRNA of claim 63 or 64, wherein the shortened hairpin 2 region has a duplex portion of 4-8 base paired nucleotides in length.

66. The gRNA of any one of claims 63-65, wherein the shortened hairpin 2 region has a duplex portion of 4-6 base paired nucleotides in length.

67. The gRNA of any one of claims 36-66, wherein the upper stem of the shortened hairpin 2 region comprises one, two, three, or four base pairs.

68. The gRNA of any one of claims 36-67, wherein all of positions 113-121 and 126-134 of the shortened hairpin 2 region are deleted.

69. The gRNA of any one of claims 36-68, wherein one or more of nucleotides 113-134 of the shortened hairpin 2 region is substituted relative to SEQ ID NO: 500.

70. The gRNA of any one of claims 36-69, wherein the third internal linker substitutes for at least part of or for all of nucleotides 122-125.

71. The gRNA of any one of claims 36-70, wherein the third internal linker substitutes for at least part of or for all of nucleotides 112-135.

72. The gRNA of any one of claims 36-71, wherein the shortened hairpin 2 region has 2-15 modified nucleotides.

73. The gRNA of any one of claims 1-72, wherein the guide region of the gRNA comprises at least two modified nucleotides, optionally at least four modified nucleotides.

74. The gRNA of any one of claims 1-73,
1) comprising a 3′ end modification, and comprising a modification in the upper stem region of the repeat/anti-repeat region;
2) comprising a 3′ end modification, and a modification in the hairpin 1 region; or
3) comprising a 3′ end modification, and a modification in the hairpin 2 region.

75. The gRNA of any one of claims 1-74,
1) comprising a 5′ end modification, and comprising a modification in the upper stem region of the repeat/anti-repeat region;
2) comprising a 5′ end modification, and a modification in the hairpin 1 region;
3) comprising a 5′ end modification, and a modification in the hairpin 2 region;
4) comprising a 5′ end modification, a modification in the upper stem region of the repeat/anti-repeat region, and a 3′ end modification;
5) comprising a 5′ end modification, a modification in the hairpin 1 region, and a 3′ end modification;
6) comprising a 5′ end modification, a modification in the hairpin 1 region, a modification in the hairpin 2 region, and a 3′ end modification; or
7) comprising a 5′ end modification, a modification in the repeat/anti-repeat region, a modification in the hairpin 1 region, a modification in the hairpin 2 region, and a 3′ end modification.

76. The gRNA of any one of claims 74-75, wherein the modification in the repeat/anti-repeat region, the hairpin 1 region, or the hairpin 2 region comprises a modified nucleotide selected from 2′-O-methyl (2′-OMe) modified nucleotide and a phosphorothioate (PS) linkage between nucleotides, or combinations thereof.

77. The gRNA of any one of claims 1-76, wherein nucleotides 1-3 of the guide region are modified and nucleotides in the guide region other than nucleotides 1-3 are not modified.

78. A single guide RNA (sgRNA) comprising any one of SEQ ID NOs: 1001-1012 or any other sequences as shown in Table 4A.

79. The gRNA of any one of claims 1-78, comprising a nucleotide sequence having at least 99, 98, 97, 96, 95, 94, 93, 92, 91, 90, 85, 80, 75, or 70% identity to the nucleotide sequence of any one of SEQ ID Nos: 1001-1012 or any other sequences as shown in Table 4A.

80. The gRNA of any one of claims 1-79, comprising a nucleotide sequence having at least 99, 98, 97, 96, 95, 94, 93, 92, 91, 90, 85, 80, 75, or 70% identity to the nucleotide sequence of any one of SEQ ID Nos: 1001-1002 and 710-759 as shown in Tables 4A-4B, wherein the modification at each nucleotide of the gRNA that corresponds to a nucleotide of the reference sequence identifier in Table 4A is identical to or equivalent to the modification shown in the reference sequence identifier in Table 4B.

81. The gRNA of any one of claims 1-80, comprising a nucleotide sequence having at least 99, 98, 97, 96, 95, 94, 93, 92, 91, or 90% identity to the sequence from X to the 3′ end of the nucleotide sequence of any one of SEQ ID Nos: 1001-1002 and 710-759 as shown in Tables 4A-4B, where X is the first nucleotide of the conserved region.

82. The gRNA of any one of claims 1-42 and 44-81, further comprising a 3′ tail comprising a 2′-O-Me modified nucleotide.

83. The gRNA of any one of claims 1-82, wherein the gRNA directs a nuclease to a target sequence for binding.

84. The gRNA of any one of claims 1-83, wherein the gRNA directs a nuclease to a target sequence for inducing a double-strand break within the target sequence.

85. The gRNA of any one of claims 1-84, wherein the gRNA directs a nuclease to a target sequence for inducing a single-strand break within the target sequence.

86. The gRNA of any one of claims 82-84, wherein the nuclease is a NmeCas9.

87. The composition of claim 86, wherein the Nine Cas9 is an Nme1 Cas9, an Nme2 Cas9, or an Nme3 Cas9.

88. The gRNA of any one of claims 1-87, wherein the gRNA comprising a conservative substitution, e.g., to preserve base pairing.

89. The gRNA of any one of claims 1-88, wherein the internal linker has a bridging length of about 6 Angstroms-37 Angstroms.

90. The gRNA of any one of claims 1-89, wherein the internal linker comprises 1-10 ethylene glycol subunits covalently linked to each other.

91. The gRNA of any one of claims 1-90, wherein the internal linker comprises at least two ethylene glycol subunits covalently linked to each other.

92. The gRNA of any one of claims 1-91, wherein the internal linker comprises 3 ethylene glycol subunits covalently linked to each other.

93. The gRNA of any one of claims 1-92, wherein the internal linker comprises 6 ethylene glycol subunits covalently linked to each other.

94. The gRNA of any one of claims 1-93, wherein the internal linker comprises a structure of formula (I):
˜-L0-L1-L2-# (I)
wherein:
˜ indicates a bond to a 3′ substituent of the preceding nucleotide;
#indicates a bond to a 5′ substituent of the following nucleotide;
L0 is null or C₁₋₃ aliphatic;
L1 is -[E¹-(R¹)]ₘ—, where
each R¹ is independently a C₁₋₅ aliphatic group, optionally substituted with 1 or 2 E²,
each E¹ and E² are independently a hydrogen bond acceptor, or are each independently chosen from cyclic hydrocarbons, and heterocyclic hydrocarbons, and
each m is 1, 2, 3, 4, 5, 6, 7, 8, 9, or 10; and
L2 is null, C₁₋₃ aliphatic, or is a hydrogen bond acceptor.

95. The gRNA of claim 94, wherein the internal linker comprises a PEG-linker.

96. The gRNA of claim 94 or claim 95, wherein the internal linker comprises a PEG-linker having from 1 to 10 ethylene glycol units.

97. The gRNA of any one of claims 94-96, wherein the internal linker comprises a PEG-linker having from 3 to 6 ethylene glycol units.

98. The gRNA of any one of claims 94-97, wherein the internal linker comprises a PEG-linker having 3 ethylene glycol units.

99. The gRNA of any one of claims 94-97, wherein the internal linker comprises a PEG-linker having 6 ethylene glycol units.

100. The gRNA of any one of claims 1-99, wherein the gRNA is a short guide RNA comprising a shortened conserved portion, and the internal linker substitutes for at least 2, 3, 4, 5, 6, 7, 8, 9, 10, 11, or 12 nucleotides.

101. The gRNA of any one of claims 1-35 or 78-100, wherein the gRNA is a short-single guide RNA (short-sgRNA) comprising a conserved portion of an sgRNA comprising a hairpin region, wherein the hairpin region lacks at least 5-10 nucleotides.

102. The gRNA of claim 101, wherein the at least 5-10 lacking nucleotides are consecutive.

103. The gRNA of claim 101 or 102, wherein the at least 5-10 lacking nucleotides
i. are within hairpin 1;
ii. are within hairpin 1 and the “N” between hairpin 1 and hairpin 2 relative to SEQ ID NO: 400;
iii. are within hairpin 1 and the two nucleotides immediately 3′ of hairpin 1;
iv. include at least a portion of hairpin 1;
v. are within hairpin 2;
vi. include at least a portion of hairpin 2;
vii. are within hairpin 1 and hairpin 2;
viii. include at least a portion of hairpin 1 and include the “N” between hairpin 1 and hairpin 2 relative to SEQ ID NO: 400;
ix. include at least a portion of hairpin 2 and include the “N” between hairpin 1 and hairpin 2 relative to SEQ ID NO: 400;
x. include at least a portion of hairpin 1, include the “N” between hairpin 1 and hairpin 2 relative to SEQ ID NO: 400, and include at least a portion of hairpin 2;
xi. are within hairpin 1 or hairpin 2, optionally including the “N” between hairpin 1 and hairpin 2 relative to SEQ ID NO: 400;
xii. are consecutive;
xiii. are consecutive and include the “N” between hairpin 1 and hairpin 2 relative to SEQ ID NO: 400;
xiv. are consecutive and span at least a portion of hairpin 1 and a portion of hairpin 2;
xv. are consecutive and span at least a portion of hairpin 1 and the “N” between hairpin 1 and hairpin 2 relative to SEQ ID NO: 400; or
xvi. are consecutive and span at least a portion of hairpin 1 and two nucleotides immediately 3′ of hairpin 1.

104. The gRNA of any one of claims 1-35 or 78-103, wherein the gRNA is a short-single guide RNA (short-sgRNA) comprising a conserved portion of an sgRNA comprising a hairpin region, wherein the hairpin region lacks at least 5-10 nucleotides and wherein the short-sgRNA comprises a 5′ end modification or a 3′ end modification.

105. The gRNA of any one of claims 1-35 or 78-104, wherein the at least 5-10 nucleotides comprise nucleotides 54-61 of SEQ ID NO:400, nucleotides 53-60 of SEQ ID NO:400; or nucleotides 54-58 of SEQ ID NO:400, optionally wherein the short-sgRNA comprises modifications at least H1-1 to H1-5 and H2-1 to H2-12.

106. The gRNA of any one of claims 1-35 or 78-105, comprising a shortened hairpin 1 region or a substituted and optionally shortened hairpin 1 region, wherein
(i) at least one of the following pairs of nucleotides are substituted in the substituted and optionally shortened hairpin 1 with Watson-Crick pairing nucleotides: H1-1 and H1-12, H1-2 and H1-11, H1-3 and H1-10, or H1-4 and H1-9, and the hairpin 1 region optionally lacks
(aa) any one or two of H1-5 through H1-8,
(bb) one, two, or three of the following pairs of nucleotides: H1-1 and H1-12, H1-2 and H1-11, H1-3 and H1-10 or H1-4 and H1-9, or
(cc) 1-8 nucleotides of the hairpin 1 region; or
(ii) the shortened hairpin 1 region lacks 6-8 nucleotides, preferably 6 nucleotides; and
(aa) one or more of positions H1-1, H1-2, or H1-3 is deleted or substituted relative to SEQ ID NO: 400 or
(bb) one or more of positions H1-6 through H1-10 is substituted relative to SEQ ID NO: 400; or
(iii) the shortened hairpin 1 region lacks 5-10 nucleotides, preferably 5-6 nucleotides, and one or more of positions N18, H1-12, or n is substituted relative to SEQ ID NO: 400.

107. The gRNA of any one of claims 1-35 or 78-106, comprising a shortened upper stem region, wherein the shortened upper stem region lacks 1-6 nucleotides and wherein the 6, 7, 8, 9, 10, or 11 nucleotides of the shortened upper stem region include less than or equal to 4 substitutions relative to SEQ ID NO: 400.

108. The gRNA of any one of claims 1-35 or 78-107, comprising a substitution relative to SEQ ID NO: 400 at any one or more of LS6, LS7, US3, US10, B3, N7, N15, N17, H2-2 and H2-14, wherein the substituent nucleotide is neither a pyrimidine that is followed by an adenine, nor an adenine that is preceded by a pyrimidine.

109. The gRNA of claim 106, wherein the shortened and substituted hairpin 1 lacks 1-4 nucleotides and nucleotides H1-4 through H1-9 are substituted by an internal linker.

110. The gRNA of claim 106, wherein the shortened and substituted hairpin 1 lacks one or two of the following pairs of nucleotides: H1-1 and H1-12, H1-2 and H1-11, or H1-3 and H1-10; and nucleotides H1-4 through H1-9 are substituted by an internal linker.

111. The gRNA of any one of claims 1-35 or 78-110, comprising an upper stem region, wherein the upper stem modification comprises a modification to any one or more of US1-US12 in the upper stem region.

112. The gRNA of any one of claims 1-35 or 78-111, comprising a shortened upper stem region, wherein the shortened upper stem region lacks 1-6 nucleotides.

113. The gRNA of any one of claims 1-35 or 78-111, comprising a shortened upper stem region, wherein the shortened upper stem region lacks 7-10 nucleotides and 2 nucleotides are substituted by an internal linker. The gRNA of claim 381, wherein the stem does not comprise an upper stem duplex portion.

114. The gRNA of claim 112 or 113 wherein the internal linker has a bridging length of about 3-30 atoms, optionally 12-21 atoms, 6-18 atoms, or 6-12 atoms.

115. The gRNA of any one of claims 1-114, wherein the gRNA comprises a modification.

116. The guide RNA of claim 115, wherein the modification comprises a 2′-O-methyl (2′-O-Me) modified nucleotide, a 2′-F modified nucleotide, 2′-H modified nucleotide (DNA), a 2′-O,4′-C-ethylene modified nucleotides (ENA), locked nucleotide (LNA), or unlocked nucleotide (UNA).

117. The guide RNA of claim 115 or 116, wherein the modification comprises a phosphorothioate (PS) bond between nucleotides.

118. The guide RNA of claim 117, wherein
(i) the guide RNA is a sgRNA and the modification, comprises a modification at one or more of the five nucleotides at the 5′ end of the guide RNA;
(ii) the guide RNA is a sgRNA and the modification, comprises a modification at one or more of the five nucleotides at the 3′ end of the guide RNA;
(iii) the guide RNA is a sgRNA and the modification, comprises a PS bond between each of the four nucleotides at the 5′ end of the guide RNA;
(iv) the guide RNA is a sgRNA and the modification, comprises a PS bond between each of the four nucleotides at the 3′ end of the guide RNA;
(v) the guide RNA is a sgRNA and the modification, comprises a 2′-O-Me modified nucleotide at each of the first three nucleotides at the 5′ end of the guide RNA; or
(vi) the guide RNA is a sgRNA and the modification, comprises a 2′-O-Me modified nucleotide at each of the last three nucleotides at the 3′ end of the guide RNA.

119. The gRNA of any one of claims 1-118, wherein the 3′ nucleotide of the gRNA is a nucleotide comprising a uracil or a modified uracil.

120. The gRNA of any one of claims 1-119, wherein the gRNA comprises a 3′ tail.

121. The gRNA of claim 120, wherein the 3′ tail comprises at least 1-10 nucleotides, optionally 1, 2, 3, 4, or 5 nucleotides.

122. The gRNA of claim 120 or 121, wherein the 3′ tail terminates with a nucleotide comprising a uracil or a modified uracil.

123. The gRNA of any one of claims 120-122, wherein the 3′ tail is 1 nucleotide in length.

124. The gRNA of any one of claims 120-123, wherein the 3′ tail consists of a nucleotide comprising a uracil or a modified uracil.

125. The gRNA of any one of claims 120-124, wherein the 3′ tail comprises a modification of any one or more of the nucleotides present in the 3′ tail.

126. The gRNA of any one of claims 120-125, wherein the modification of the 3′ tail is one or more of 2′-O-methyl (2′-OMe) modified nucleotide and a phosphorothioate (PS) linkage between nucleotides.

127. The gRNA of claim 125 or 126, wherein the 3′ tail is fully modified.

128. The gRNA of any one of claims 1-119, wherein the gRNA does not comprise a 3′ tail.

129. The gRNA of any one of claims 1-129, wherein the gRNA comprises a 3′ end modification or 5′ end modification.

130. The gRNA of claim 129, wherein the 3′ or 5′ end modification comprises or further comprises a 2′-O-methyl (2′-Ome) modified nucleotide.

131. The gRNA of claim 129 or claim 130, wherein the 3′ or 5′ end modification comprises or further comprises a phosphorothioate (PS) linkage between nucleotides.

132. The gRNA of any one of claims 1-131, comprising a modification in a or the hairpin region.

133. The gRNA of claim 132, comprising a modification in the hairpin region, wherein the modification in the hairpin region comprises a modified nucleotide selected from a 2′-O-methyl (2′-Ome) modified nucleotide, a 2′-fluoro (2′-F) modified nucleotide, a phosphorothioate (PS) linkage between nucleotides, or a combination thereof.

134. The gRNA of claim 132 or 133, wherein the modification in the hairpin region comprises or further comprises a 2′-O-methyl (2′-Ome) modified nucleotide.

135. The gRNA of any one of claims 132-134, wherein the modification in the hairpin region comprises or further comprises a 2′-fluoro (2′-F) modified nucleotide.

136. The gRNA of any one of claims 1-135, comprising a modification in a or the upper stem region.

137. The gRNA of claim 136, wherein the upper stem modification comprises any one or more of:
i. a modification of any one or more of US1-US12 in the upper stem region (corresponding to nucleotides 9-20 of SEQ ID NO: 400); and
ii. a modification of at least 1, 2, 3, 4, 5, 6, 7, 8, 9, 10, 11, or all 12 nucleotides in the upper stem region.

138. The gRNA of claim 136 or 137, wherein the upper stem modification comprises one or more of:
i. a 2′-OMe modified nucleotide;
ii. a 2′-O-moe modified nucleotide;
iii. a 2′-F modified nucleotide;
iv. 2′-H modified nucleotide (DNA);
v. a 2′-0,4′-C-ethylene modified nucleotides (ENA);
vi. locked nucleotide (LNA);
vii. unlocked nucleotide (UNA); and
viii. combinations of one or more of (i.)-(iii.).

139. A single guide RNA (sgRNA) comprising any one of SEQ ID NOs: 211-230 or any other sequences as shown in Tables 2A-2C.

140. The gRNA of any one of claims 1-139, comprising a nucleotide sequence having at least 99, 98, 97, 96, 95, 94, 93, 92, 91, 90, 85, 80, 75, or 70% identity to the nucleotide sequence of any one of SEQ ID Nos: 211-230 or any other sequences as shown in Tables 2A-2C.

141. The gRNA of any one of claims 1-140, comprising a nucleotide sequence having at least 99, 98, 97, 96, 95, 94, 93, 92, 91, 90, 85, 80, 75, or 70% identity to the nucleotide sequence of any one of SEQ ID NOs: 101-108, 120-175, 177-184, 211-230 as shown in Tables 2A-2C, wherein the modification at each nucleotide of the gRNA that corresponds to a nucleotide of the reference sequence identifier in Table 2C is identical to or equivalent to the modification shown in the reference sequence identifier in Table 2A or 2B.

142. The gRNA of any one of claims 1-141, comprising a nucleotide sequence having at least 99, 98, 97, 96, 95, 94, 93, 92, 91, or 90% identity to the sequence from X to the 3′ end of the nucleotide sequence of any one of SEQ ID NOs: 101-108, 120-175, 177-184, and 211-230 as shown in Tables 2A-2C, where X is the first nucleotide of the conserved region.

143. A composition comprising a gRNA of any one of claims 1-142 associated with a lipid nanoparticle (LNP).

144. A composition comprising the gRNA of any one of claims 1-142, or the composition of claim 143, further comprising a nuclease or an mRNA which encodes the nuclease.

145. An LNP composition comprising a gRNA of any one of claims 1-142.

146. A LNP composition comprising a gRNA of any one of claims 14-35 and 73-142 and an mRNA encoding SpyCas9.

147. The LNP composition of claim 145 or claim 146, wherein the LNP comprises (9z,12z)-3-((4,4-bis(octyloxy)butanoyl)oxy)-2-((((3-(diethylamino)propoxy)carbonyl)oxy)methyl)propyl octadeca-9,12-dienoate or nonyl 8-((7,7-bis(octyloxy)heptyl)(2-hydroxyethyl)amino)octanoate.

148. The composition of any one of claims 145-147, wherein the LNP comprises a molar ratio of a cationic lipid amine to RNA phosphate (N:P) of about 4.5-6.5, optionally the N:P of about 6.0.

149. The composition of claim 144-148, wherein the nuclease comprises a protein or a nucleic acid encoding the nuclease.

150. The composition of claim 149, wherein the nuclease is a Cas nuclease.

151. The composition of claim 150, wherein the Cas nuclease is a Cas9.

152. The composition of claim 151, wherein the Cas9 is S. pyogenes Cas9 (SpyCas9).

153. The composition of claim 151, wherein the Cas9 is a NmeCas9, optionally wherein the NmeCas9 an Nme1Cas9, an Nme2Cas9, or an Nme3Cas9.

154. The composition of any one of claims 144-153, wherein the nuclease is a cleavase, a nickase, or a catalytically inactive nuclease, or is a fusion protein comprising a deaminase.

155. The composition of any one of claim 144-154, wherein the nucleic acid encoding the nuclease is selected from:
a. a DNA coding sequence;
b. an mRNA with an open reading frame (ORF);
c. a coding sequence in an expression vector;
d. a coding sequence in a viral vector.

156. The composition of claim 155, wherein the mRNA comprises the sequence of any one of SEQ ID NOs: 321-323, 361, 363-372, and 374-382.

157. A pharmaceutical formulation comprising the gRNA of any one of claims 1-142, or the composition of any one of claims 143-156 and a pharmaceutically acceptable carrier.

158. A method of modifying a target DNA comprising, delivering to a cell any one or more of the following:
i. the gRNA of any one of claims 1-142;
ii. the composition of any one of claims 143-156; and
iii. the pharmaceutical formulation of claim 157.

159. The method of claim 158, wherein the method results in an insertion or deletion in a gene.

160. The method of claim 158 or 159, further comprising delivering to the cell a template, wherein at least a part of the template incorporates into a target DNA at or near a double strand break site induced by the Cas protein.

161. The gRNA of any one of claims 1-142, the composition of claims 143-156, or the pharmaceutical formulation of claim 157 for use in preparing a medicament for treating a disease or disorder.

162. Use of the gRNA of any one of claims 1-142, the composition of claims 143-156, or the pharmaceutical formulation of claim 157 in the manufacture of a medicament for treating a disease or disorder.

163. A composition comprising the gRNA of any one of claims 1-142, wherein the composition does not comprise an unlinked portion of the gRNA.

164. A solid support covalently attached to the linker of the gRNA of any one of claims 1-142.

165. A method of synthesizing a gRNA comprising an internal linker wherein it is a single synthetic process.

166. A method of synthesizing a gRNA wherein an internal linker is incorporated in line during synthesis.

167. A method of synthesizing a gRNA using a series of sequential coupling reactions wherein the reactions comprise:
a) coupling reaction for covalent linkage of a first nucleotide to a second nucleotide;
b) a coupling reaction for covalent linkage of an internal linker to the second nucleotide; and
c) a coupling reaction for covalent linkage of a third nucleotide to the internal linker, wherein the coupling reaction for the covalent linkages are all the same.

168. The method of claim 167, wherein covalent linkage is performed using phosphoramidite chemistry.
